(19) United States
(12) Patent Application Publication
(10) Pub. No.: US 2021/0300940 A1
(43) Pub. Date: Sep. 30, 2021

(54) TLR7/8 ANTAGONISTS AND USES THEREOF

(71) Applicant: Merck Patent GmbH, Darmstadt (DE)

(72) Inventors: Brian Sherer, Nashua, NH (US); Ruoxi Lan, Waltham, MA (US); Nadia Brugger, Cambridge, MA (US); Xiaoling Chen, Chestnut Hill, MA (US); Momar Toure, Billerica, MA (US); Esther Cleary, Somerville, MA (US); Lizbeth Celeste Deselm, Melrose, MA (US); Yanping Wang, Billerica, MA (US)

(73) Assignee: Merck Patent GmbH, Darmstadt (DE)

(21) Appl. No.: 17/250,489

(22) PCT Filed: Jul. 29, 2019
(86) PCT No.: PCT/EP2019/070312

§ 371 Date: Jan. 27, 2021

Related U.S. Application Data
(60) Provisional application No. 62/712,439, filed on Jul. 31, 2018.

(51) Int. Cl. C07D 495/10; C07D 401/14; C07D 401/04; C07D 471/04; C07D 409/14; C07D 405/14; C07D 413/14; C07D 471/10; C07D 417/14

(52) U.S. Cl. CPC C07D 495/10; C07D 401/14; C07D 401/04; C07D 471/04; C07D 417/14; C07D 405/14; C07D 413/14; C07D 471/10; C07D 409/14

(57) ABSTRACT

Compounds of Formula (I) and pharmaceutically acceptable compositions thereof are useful as TLR7/8 antagonists.

### TECHNICAL FIELD OF THE INVENTION

The present invention provides for compounds of Formula (I) as toll-like receptor 7/8 (TLR7/8) antagonists and their use in the treatment of immune disorders, and other diseases related to TLR7/8 overexpression.

### BACKGROUND OF THE INVENTION

Toll-like receptors (TLR) currently comprising a gene family of 10 receptors with different specificities are part of the cellular pathogen pattern recognition system, which has evolved for defense against a variety of infections (bacteria, virus, fungi). Activation of TLRs leads to cytokine responses, e.g. with release of interferons and activation of specified immune cells. The functional expression of selected TLRs in tissues is highly different. Part of the receptors are located at the cell surface such as TLR4 (stimulated by E. coli lipopolysaccharide LPS), e.g. on epithelial cells, or TLR3, 7, 8 and 9 located at endosomal membranes in specified immune cells. The latter are all activated by nucleic acids, but recognize various types of them. For instance, TLR9 is activated by single stranded DNA containing CpG subsequences, TLR7 and 8 are activated by single stranded RNA, and TLR3 is activated by double-stranded RNA.

TLRs have been implicated in various autoimmune and inflammatory diseases, with the clearest example being the role played by TLR7 in the pathogenesis of systemic lupus erythematosus (Barrat and Coffman, Immunol Rev, 223:271-283, 2008). Additionally, a TLR8 polymorphism has been associated with rheumatoid arthritis (Enevold et al., J Rheumatol, 37:905-10, 2010). Although various TLR7, TLR8 and TLR9 inhibitors have been described, additional TLR inhibitors are desirable. In particular, polynucleotides having inhibitory motifs for one or more of TLR7, TLR8 and TLR9 are needed to precisely inhibit an immune response in a subject (e.g., patient having an autoimmune disease or an inflammatory disorder).

For several years strong efforts are ongoing worldwide trying to exploit the strong immune activation induced by TLR7, 8 or 9 agonists for the treatment of cancer. Cancer immunotherapy, however, experienced a long history of failures. In recent years, though, the knowledge on cancer immune surveillance and the function of subsets of immune cells thereby was improved drastically. TLR7 or TLR9 agonists are in clinical development for cancer mono- or combination therapies, or as vaccine adjuvant. The TLR agonist approach for cancer immunotherapy is different from earlier efforts using, e.g. cytokines, interferons or monovalent vaccinations. TLR agonist mediated immune activation is pleiotropic via specified immune cells (primarily dendritic cells and B-cells, subsequently other cells), which generates an innate and adaptive immune response. Moreover, not only one interferon is induced, but rather the many different isoform's altogether, and not only type I (alpha, beta), but also (indirectly) type II (gamma, NK cells).

### SUMMARY OF THE INVENTION

In one aspect, the invention provides compounds of Formula (I):

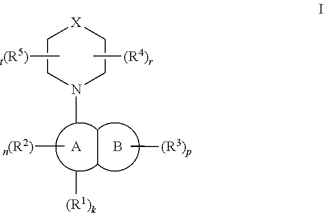

and pharmaceutically acceptable derivatives, solvates, salts, hydrates and stereoisomers thereof.

In another aspect, the invention provides compounds of Formula (I) which are dual antagonists of TLR7 and TLR8. In another aspect, the invention provides compounds of Formula (I) which are suitable for the treatment and/or prevention of disorders related to TLR7/8. In another aspect, the invention provides compounds which are able to modulate, especially inhibit the activity or function of TLR7/8 in disease states in mammals, especially in humans. In certain embodiments, the compounds are non-brain penetrant compounds. In certain embodiments, the compounds are non-brain penetrant compounds, due to the structure of the compounds of the invention.

According to another aspect of the invention are provided methods for the treatment and/or prevention of auto-immune disorders.

According to another aspect, the present invention provides compounds of Formula (I) which are selective for TLR7 or TLR8.

According to another aspect, the present invention provides compounds of Formula (I) which are selective for TLR7 and TLR8.

### DETAILED DESCRIPTION OF CERTAIN EMBODIMENTS

### 1. General Description of Compounds of the Invention

In certain aspects, the present invention provides for antagonists of TLR7/8. In some embodiments, such compounds include those of the formulae described herein, or a pharmaceutically acceptable salt thereof, wherein each variable is as defined and described herein.

### 2. Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Exemplary aliphatic groups are linear or branched, substituted or unsubstituted C₁-C₈ alkyl, C₂-C₈ alkenyl, C₂-C₈ alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, or phosphorus (including, any oxidized form of nitrogen, sulfur, or phosphorus; the quaternized form of any basic nitrogen or; a substitutable nitrogen of a heterocyclic ring, for example N (as in 3,4-dihydro-2H-pyrrolyl), NH (as in pyrrolidinyl) or NR⁺(as in N-substituted pyrrolidinyl)).

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” is used interchangeably with the term “aryl ring”. In certain embodiments of the present invention, “aryl” refers to an aromatic ring system. Exemplary aryl groups are phenyl, biphenyl, naphthyl, anthracyl and the like, which optionally includes one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group is optionally mono- or bicyclic. The term “heteroaryl” is used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen is N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group is optionally mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, certain compounds of the invention contain “optionally substituted” moieties. In general, the term “substituted”, whether preceded by the term “optionally” or not, means that one or more hydrogens of the designated moiety are replaced with a suitable substituent. “Substituted” applies to one or more hydrogens that are either explicit or implicit from the structure (e.g.,

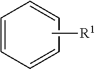

refers to at least

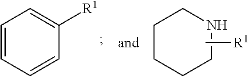

refers to at least

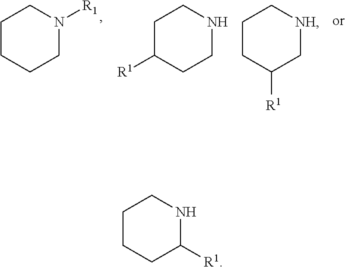

Unless otherwise indicated, an “optionally substituted” group has a suitable substituent at each substitutable position of the group, and when more than one position in any given structure is substituted with more than one substituent selected from a specified group, the substituent is either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently deuterium; halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which are optionally substituted with R^{∘}; —(CH₂)₀₋₄O(CH₂)₀₋₁Ph which is optionally substituted with R^{∘}; —CH═CHPh, which is optionally substituted with R^{∘}; —(CH₂)₀₋₄O(CH₂)₀₋₁-pyridyl which is optionally substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} is optionally substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which is optionally substituted as defined below.

Suitable monovalent substituents on R^{∘} (or the ring formed by taking two independent occurrences of R^{∘} together with their intervening atoms), are independently deuterium, halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which is substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which is optionally substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which is optionally substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

In certain embodiments, the terms “optionally substituted”, “optionally substituted alkyl,” “optionally substituted “optionally substituted alkenyl,” “optionally substituted alkynyl”, “optionally substituted carbocyclic,” “optionally substituted aryl”, “optionally substituted heteroaryl,” “optionally substituted heterocyclic,” and any other optionally substituted group as used herein, refer to groups that are substituted or unsubstituted by independent replacement of one, two, or three or more of the hydrogen atoms thereon with typical substituents including, but not limited to:
—F, —Cl, —Br, —I, deuterium,
—OH, protected hydroxy, alkoxy, oxo, thiooxo,
—NO₂, —CN, CF₃, N₃,
—NH₂, protected amino, —NH alkyl, —NH alkenyl, —NH alkynyl, —NH cycloalkyl, —NH-aryl, —NH-heteroaryl, —NH-heterocyclic, -dialkylamino, -diarylamino, -diheteroarylamino,
—O— alkyl, —O— alkenyl, —O— alkynyl, —O— cycloalkyl, —O-aryl, —O-heteroaryl, —O-heterocyclic,
—C(O)— alkyl, —C(O)— alkenyl, —C(O)— alkynyl, —C(O)— carbocyclyl, —C(O)-aryl, —C(O)— heteroaryl, —C(O)-heterocyclyl,
—CONH₂, —CONH— alkyl, —CONH— alkenyl, —CONH— alkynyl, —CONH-carbocyclyl, —CONH-aryl, —CONH-heteroaryl, —CONH-heterocyclyl,
—CO₂-alkyl, —OCO₂-alkenyl, —OCO₂-alkynyl, —OCO₂-carbocyclyl, —OCO₂-aryl, —OCO₂-heteroaryl, —OCO₂-heterocyclyl, —OCONH₂, —OCONH-alkyl, —OCONH-alkenyl, —OCONH-alkynyl, —OCONH-carbocyclyl, —OCONH-aryl, —OCONH-heteroaryl, —OCONH-heterocyclyl, —NHC(O)— alkyl, —NHC(O)— alkenyl, —NHC(O)— alkynyl, —NHC(O)— carbocyclyl, —NHC(O)-aryl, —NHC(O)-heteroaryl, —NHC(O)-heterocyclyl, —NHCO₂— alkyl, —NHCO₂— alkenyl, —NHCO₂— alkynyl, —NHCO₂— carbocyclyl, —NHCO₂-aryl, —NHCO₂— heteroaryl, —NHCO₂— heterocyclyl, —NHC(O)NH₂, —NHC(O)NH— alkyl, —NHC(O)NH— alkenyl, —NHC(O)NH— alkenyl, —NHC(O)NH— carbocyclyl, —NHC(O)NH-aryl, —NHC(O)NH-heteroaryl, —NHC(O)NH— heterocyclyl, NHC(S)NH₂, —NHC(S)NH— alkyl, —NHC(S)NH— alkenyl, —NHC(S)NH— alkynyl, —NHC(S)NH— carbocyclyl, —NHC(S)NH-aryl, —NHC(S)NH-heteroaryl, —NHC(S)NH-heterocyclyl, —NHC(NH)NH₂, —NHC(NH)NH— alkyl, —NHC(NH)NH-alkenyl, —NHC(NH)NH— alkenyl, —NHC(NH)NH— carbocyclyl, —NHC(NH)NH-aryl, —NHC(NH)NH-heteroaryl, —NHC(NH)NH— heterocyclyl, —NHC(NH)— alkyl, —NHC(NH)— alkenyl, —NHC(NH)— alkenyl, —NHC(NH)— carbocyclyl, —NHC(NH)-aryl, —NHC(NH)-heteroaryl, —NHC(NH)-heterocyclyl,
—C(NH)NH— alkyl, —C(NH)NH— alkenyl, —C(NH)NH— alkynyl, —C(NH)NH— carbocyclyl, —C(NH)NH-aryl, —C(NH)NH-heteroaryl, —C(NH)NH-heterocyclyl,
—S(O)— alkyl, —S(O)-alkenyl, —S(O)-alkynyl, —S(O)-carbocyclyl, —S(O)-aryl, —S(O)-heteroaryl, —S(O)-heterocyclyl —SO₂NH₂, —SO₂NH-alkyl, —SO₂NH-alkenyl, —SO₂NH-alkynyl, —SO₂NH-carbocyclyl, —SO₂NH-aryl, —SO₂NH-heteroaryl, —SO₂NH-heterocyclyl,
—NHSO₂— alkyl, —NHSO₂— alkenyl, —NHSO₂-alkynyl, —NHSO₂— carbocyclyl, —NHSO₂-aryl,
NHSO₂-heteroaryl, —NHSO₂-heterocyclyl,
—CH₂NH₂, —CH₂SO₂CH₃,
mono-, di-, or tri-alkyl silyl,
alkyl, -alkenyl, -alkynyl, -aryl, -arylalkyl, -heteroaryl, -heteroarylalkyl, -heterocycloalkyl, -cycloalkyl, -carbocyclic, -heterocyclic, polyalkoxyalkyl, polyalkoxy, -methoxymethoxy, -methoxyethoxy, —SH, —S— alkyl, —S— alkenyl, —S— alkynyl, —S— carbocyclyl, —S-aryl, —S-heteroaryl, —S-heterocyclyl, or methylthiomethyl.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. In some embodiments, the group comprises one or more deuterium atoms.

There is furthermore intended that a compound of the formula I includes isotope-labeled forms thereof. An isotope-labeled form of a compound of the formula I is identical to this compound apart from the fact that one or more atoms of the compound have been replaced by an atom or atoms having an atomic mass or mass number which differs from the atomic mass or mass number of the atom which usually occurs naturally. Examples of isotopes which are readily commercially available and which can be incorporated into a compound of the formula I by well-known methods include isotopes of hydrogen, carbon, nitrogen, oxygen, phos-phorus, fluo-rine and chlorine, for example ²H, ³H, ¹³C, ¹⁴C, ¹⁵N, ¹⁸O, ¹⁷O, ³¹P, ³²P, ³⁵S, ¹⁸F and ³⁶Cl, respectively. A compound of the formula I, a prodrug, thereof or a pharmaceutically acceptable salt of either which contains one or more of the above-mentioned isotopes and/or other isotopes of other atoms is intended to be part of the present invention. An isotope-labeled compound of the formula I can be used in a number of beneficial ways. For example, an isotope-labeled compound of the formula I into which, for example, a radioisotope, such as ³H or ¹⁴C, has been incorporated, is suitable for medicament and/or substrate tissue distribution assays. These radioisotopes, i.e. tritium (³H) and carbon-14 (¹⁴C), are particularly preferred owing to simple preparation and excellent detectability. Incorporation of heavier isotopes, for example deuterium (²H), into a compound of the formula I has therapeutic advantages owing to the higher metabolic stability of this isotope-labeled compound. Higher metabolic stability translates directly into an increased in vivo half-life or lower dosages, which under most circumstances would represent a preferred embodiment of the present invention. An isotope-labeled compound of the formula I can usually be prepared by carrying out the procedures disclosed in the synthesis schemes and the related description, in the example part and in the preparation part in the present text, replacing a non-isotope-labeled reactant by a readily available isotope-labeled reactant.

Deuterium (²H) can also be incorporated into a compound of the formula I for the purpose in order to manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate in rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D} 2-7 are typical. If this rate difference is successfully applied to a com-pound of the formula I that is susceptible to oxidation, the profile of this compound in vivo can be drastically modified and result in improved pharmacokinetic properties.

When discovering and developing therapeutic agents, the person skilled in the art is able to optimize pharmacokinetic parameters while retaining desirable in vitro properties. It is reasonable to assume that many compounds with poor pharmacokinetic profiles are susceptible to oxidative metabolism. In vitro liver microsomal assays currently available provide valuable information on the course of oxidative metabolism of this type, which in turn permits the rational design of deuterated compounds of the formula I with improved stability through resistance to such oxidative metabolism. Significant improvements in the pharmacokinetic profiles of compounds of the formula I are thereby obtained, and can be expressed quantitatively in terms of increases in the in vivo half-life (t/2), concentration at maximum therapeutic effect (Cₘₐₓ), area under the dose response curve (AUC), and F; and in terms of reduced clearance, dose and materials costs.

The following is intended to illustrate the above: a compound of the formula I which has multiple potential sites of attack for oxidative metabolism, for example benzylic hydrogen atoms and hydrogen atoms bonded to a nitrogen atom, is prepared as a series of analogues in which various combinations of hydrogen atoms are replaced by deuterium atoms, so that some, most or all of these hydrogen atoms have been replaced by deuterium atoms. Half-life determinations enable favorable and accurate determination of the extent of the extent to which the improvement in resistance to oxidative metabolism has improved. In this way, it is determined that the half-life of the parent compound can be extended by up to 100% as the result of deuterium-hydrogen exchange of this type.

Deuterium-hydrogen exchange in a compound of the formula I can also be used to achieve a favorable modification of the metabolite spectrum of the starting compound in order to diminish or eliminate undesired toxic metabolites. For example, if a toxic metabolite arises through oxidative carbon-hydrogen (C—H) bond cleavage, it can reasonably be assumed that the deuterated analogue will greatly diminish or eliminate production of the unwanted metabolite, even if the particular oxidation is not a rate-determining step. Further information on the state of the art with respect to deuterium-hydrogen exchange may be found, for example in Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al, Biochemistry 33(10) 2927-2937, 1994, and Jarman et al. Carcinogenesis 16(4), 683-688, 1993.

As used herein, the term “modulator” is defined as a compound that binds to and/or inhibits the target with measurable affinity. In certain embodiments, a modulator has an IC₅₀ and/or binding constant of less about 50 μM, less than about 1 μM, less than about 500 nM, less than about 100 nM, or less than about 10 nM.

The terms “measurable affinity” and “measurably inhibit,” as used herein, means a measurable change in TLR7/8 activity between a sample comprising a compound of the present invention, or composition thereof, and TLR7/8, and an equivalent sample comprising TLR7/8, in the absence of said compound, or composition thereof.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

### 3. Description of Exemplary Compounds

According to one aspect, the present invention provides a compound of formula I,

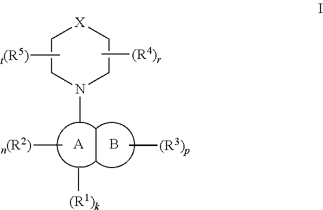

or a pharmaceutically acceptable salt thereof, wherein:
Ring A is aryl or heteroaryl having 1-4 heteroatoms independently selected from nitrogen, oxygen, or sulfur; each of which is optionally substituted;
Ring B is aryl or heteroaryl having 1-4 heteroatoms independently selected from nitrogen, oxygen, or sulfur; each of which is optionally substituted;
R¹ is -Me, —CF₃, —OMe, —OEt, or —CN;
each R² is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R³ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
X is C(R⁴)₂, O, NR⁴, S, S(R⁴), or S(R⁴)₂;
each R⁴ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R⁵ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
k is 0 or 1;
n is 0, 1, or 2;
p is 0, 1, or 2;
r is 0, 1, or 2; and
t is 0, 1, or 2.

In certain embodiments, R¹ is -Me.

In certain embodiments, R¹ is —CF₃.

In certain embodiments, R¹ is —OMe.

In certain embodiments, R¹ is —OEt.

In certain embodiments, R¹ is —CN.

In certain embodiments, Ring A is C₆ aryl or a 6 membered monocyclic heteroaryl having 1-4 heteroatoms independently selected from nitrogen, oxygen, or sulfur; each of which is optionally substituted.

In certain embodiments, Ring A is phenyl, pyridyl, pyrimidinyl, pyrazinyl, pyridazinyl, or triazinyl; each of which is optionally substituted.

In certain embodiments, Ring A is phenyl, pyridyl, or pyrimidinyl; each of which is optionally substituted.

In certain embodiments, Ring B is C₆ aryl or a 5-6 membered monocyclic heteroaryl having 1-4 heteroatoms independently selected from nitrogen, oxygen, or sulfur; each of which is optionally substituted.

In certain embodiments, Ring B is phenyl, pyridyl, pyrimidinyl, pyrazinyl, pyridazinyl, triazinyl, pyrrole, imidazole, isoxazole, oxazole, or thiazole; each of which is optionally substituted.

In certain embodiments, Ring A and Ring B is

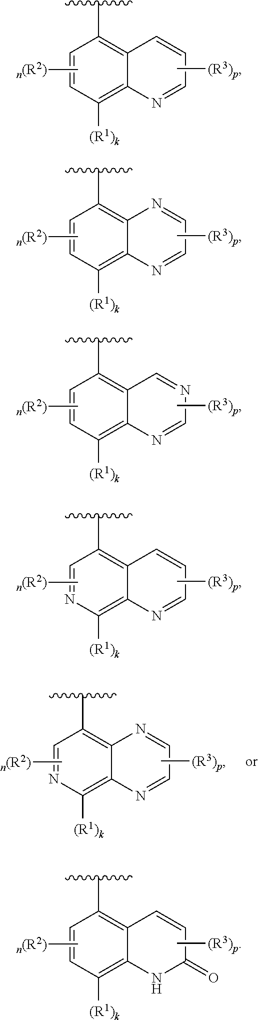

In certain embodiments, Ring A and Ring B is

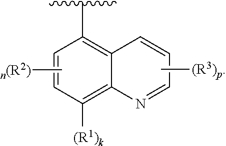

In certain embodiments, Ring A and Ring B is

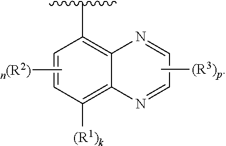

In certain embodiments, Ring A and Ring B is

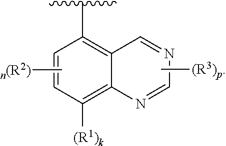

In certain embodiments, Ring A and Ring B is

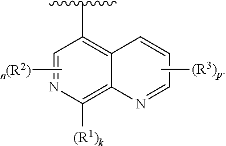

In certain embodiments, Ring A and Ring B is

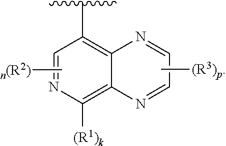

In certain embodiments, Ring A and Ring B is

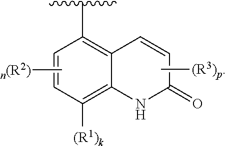

In certain embodiments, each R² is independently —H.

In certain embodiments, each R² is independently C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R² is independently methyl, ethyl, ethyl, propyl, i-propyl, butyl, s-butyl, t-butyl, straight or branched pentyl, or straight or branched hexyl; each of which is optionally substituted.

In certain embodiments, each R² is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R² is independently halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, each R² is independently —F.

In certain embodiments, each R³ is independently —H.

In certain embodiments, each R³ is independently C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R³ is independently methyl, ethyl, ethyl, propyl, i-propyl, butyl, s-butyl, t-butyl, straight or branched pentyl, or straight or branched hexyl; each of which is optionally substituted.

In certain embodiments, each R³ is independently methyl.

In certain embodiments, each R³ is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R³ is independently halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, each R³ is independently —F.

In certain embodiments, X is C(R⁴)₂ or O.

In certain embodiments, X is C(R⁴)₂. In certain embodiments, X is CH₂.

In certain embodiments, X is O.

In certain embodiments, each R⁴ is independently —H.

In certain embodiments, each R⁴ is independently C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R⁴ is independently methyl, ethyl, ethyl, propyl, i-propyl, butyl, s-butyl, t-butyl, straight or branched pentyl, or straight or branched hexyl; each of which is optionally substituted.

In certain embodiments, each R⁴ is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R⁴ is independently halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, each R⁴ is independently —H, C₁₋₆ aliphatic, —OR, —C(O)R, —CO₂R, —C(O)N(R)₂, —NRC(O)R, —NRC(O)N(R)₂, —NRSO₂R, or —N(R)₂; each of which is optionally substituted.

In certain embodiments, each R⁴ is independently —H, C₁₋₆ aliphatic, —C(O)N(R)₂, —NRC(O)R, or —N(R)₂; each of which is optionally substituted.

In certain embodiments, each R⁴ is independently

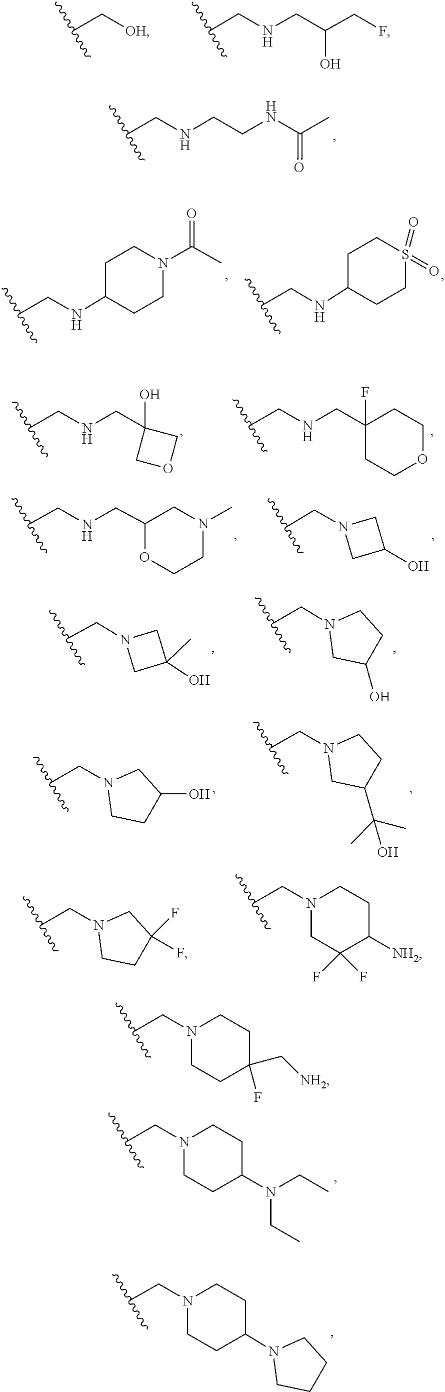

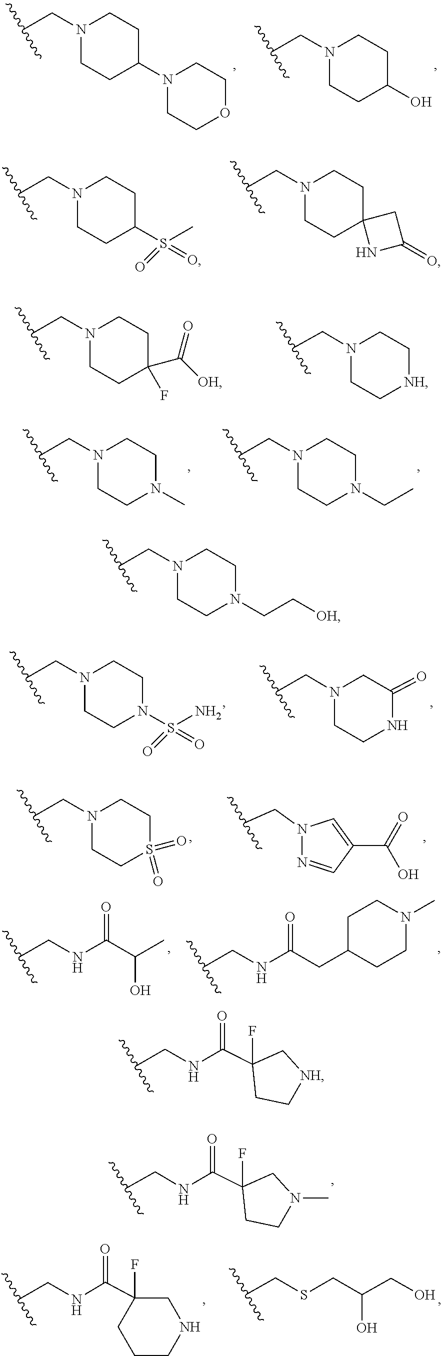

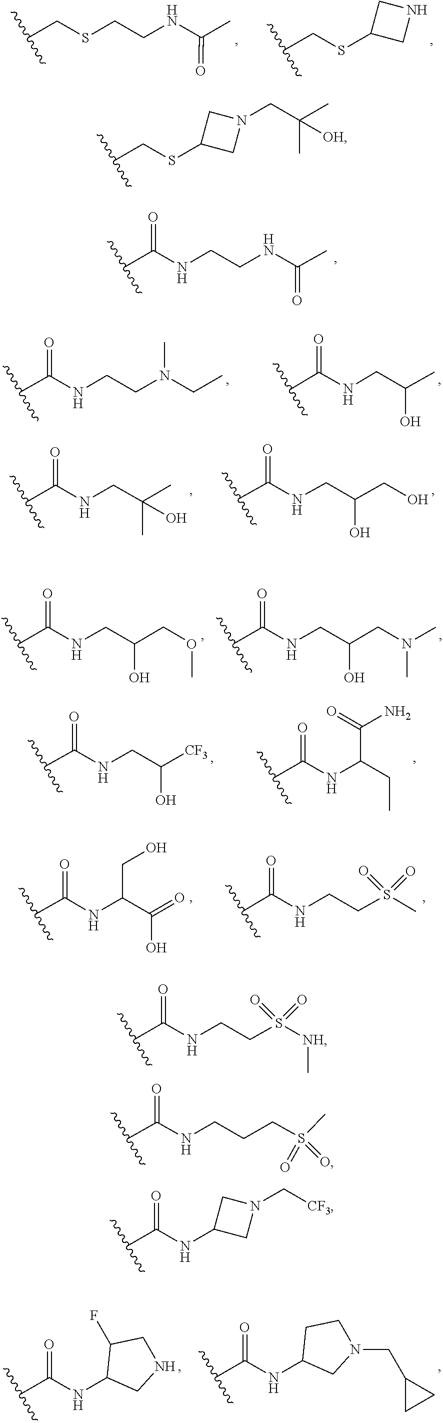

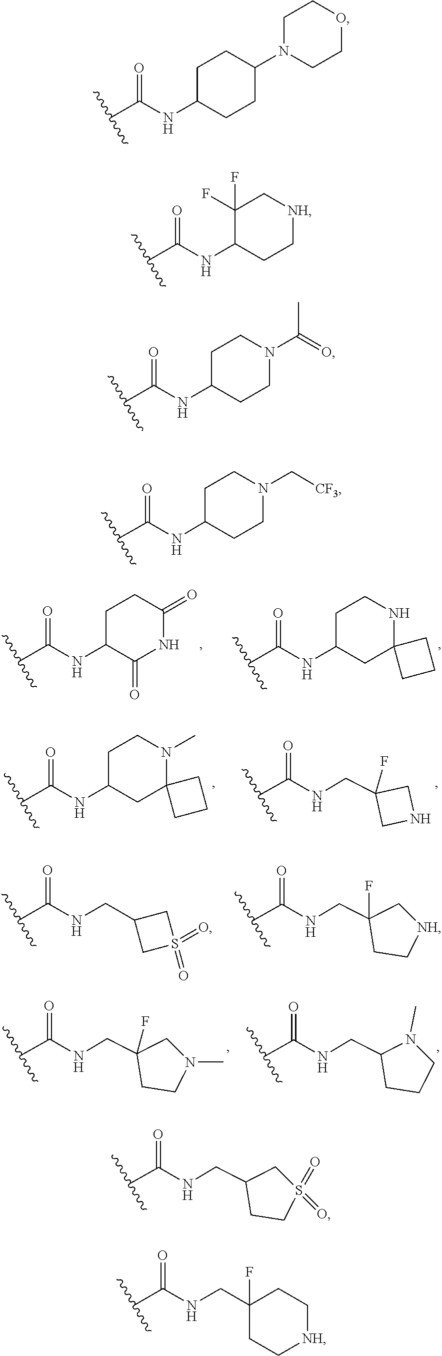

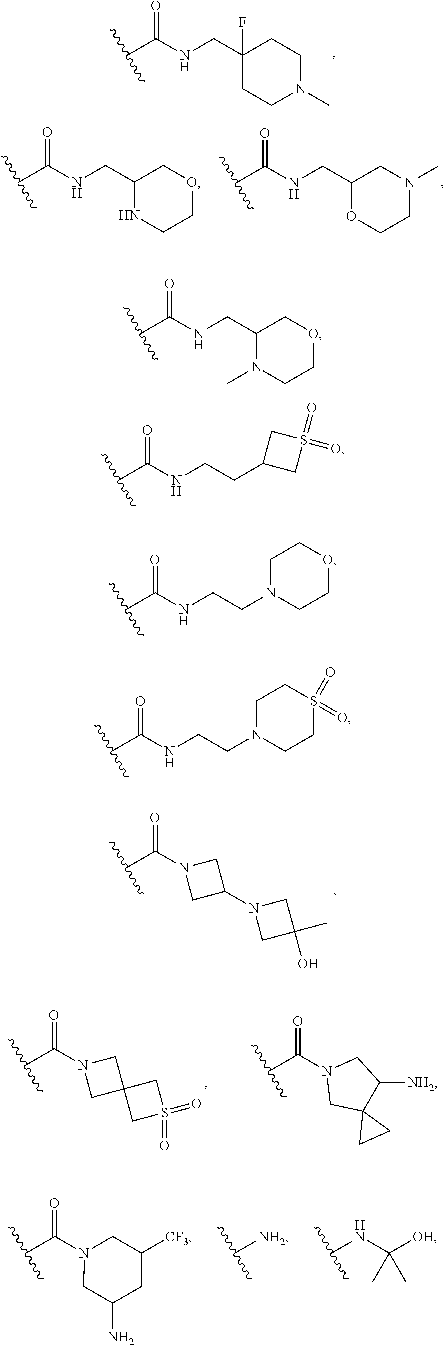

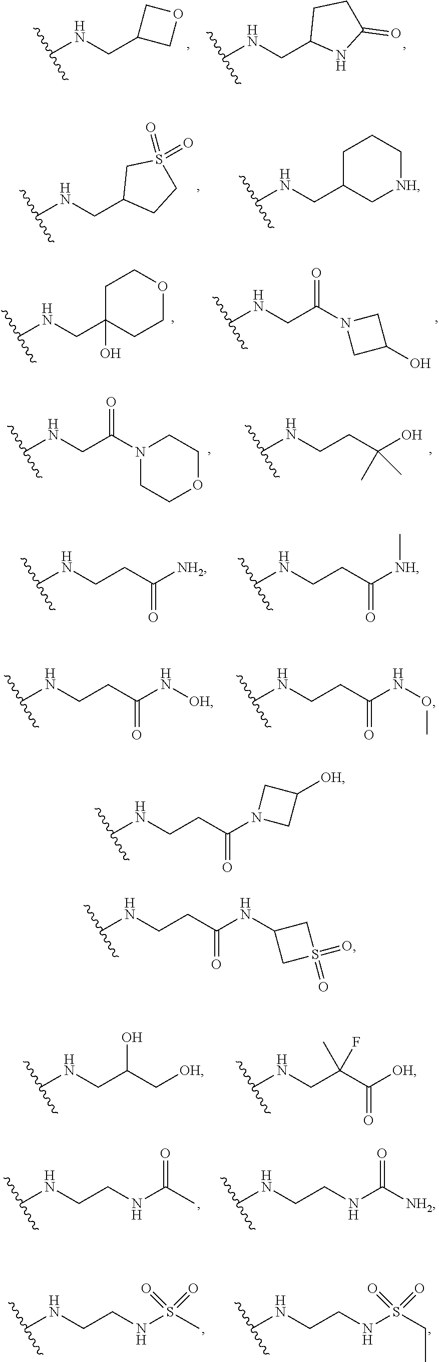

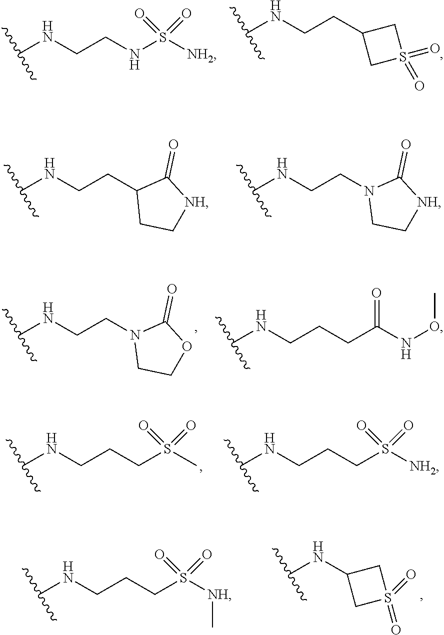

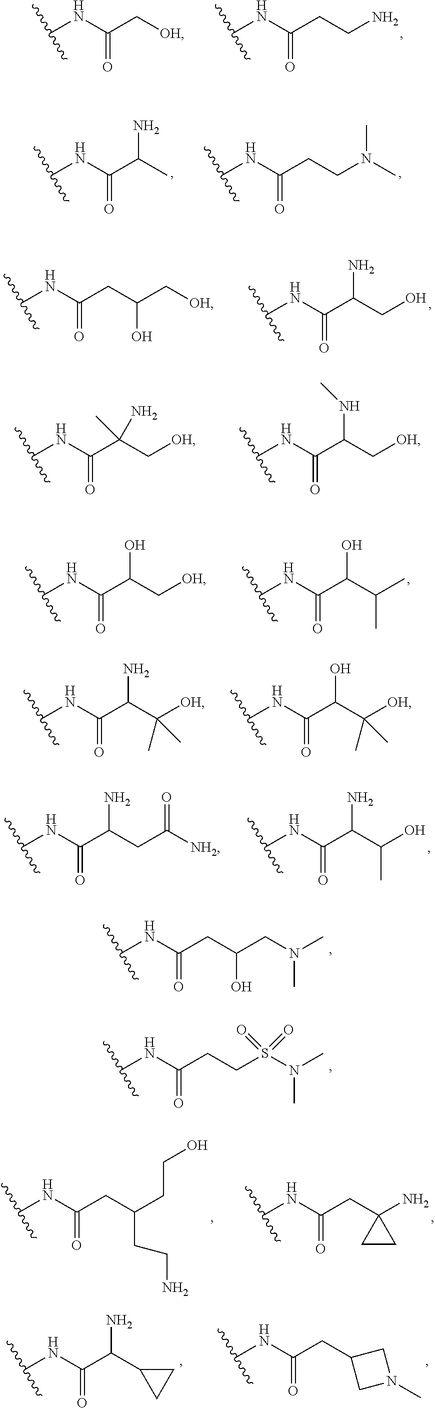

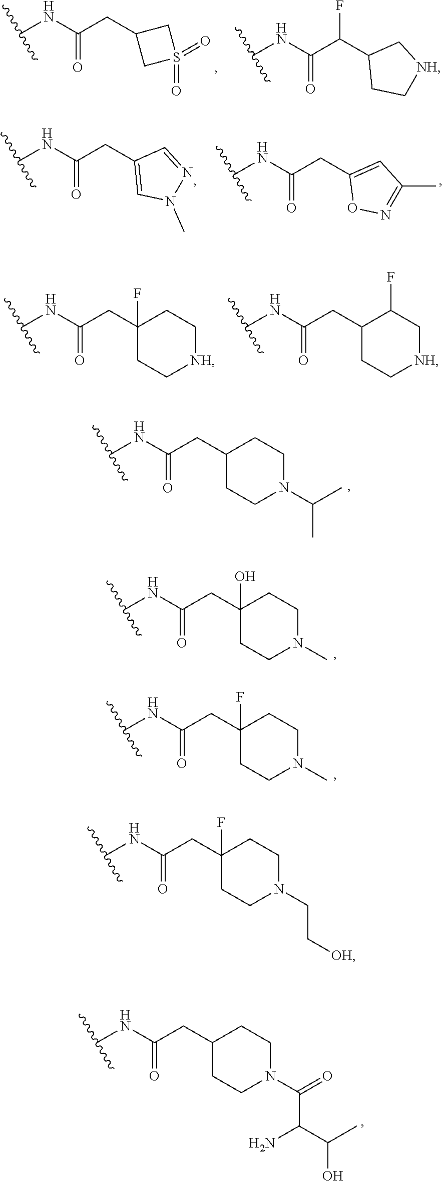

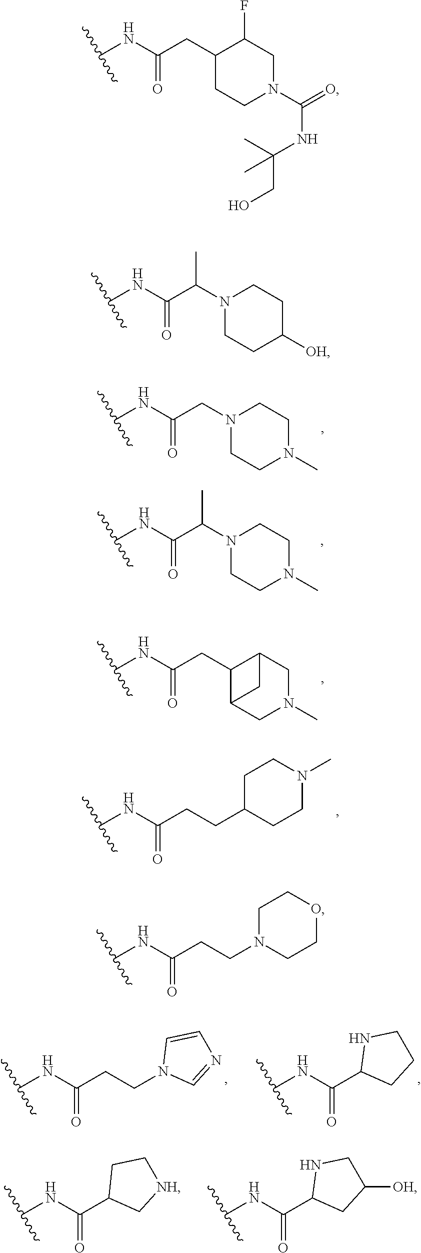

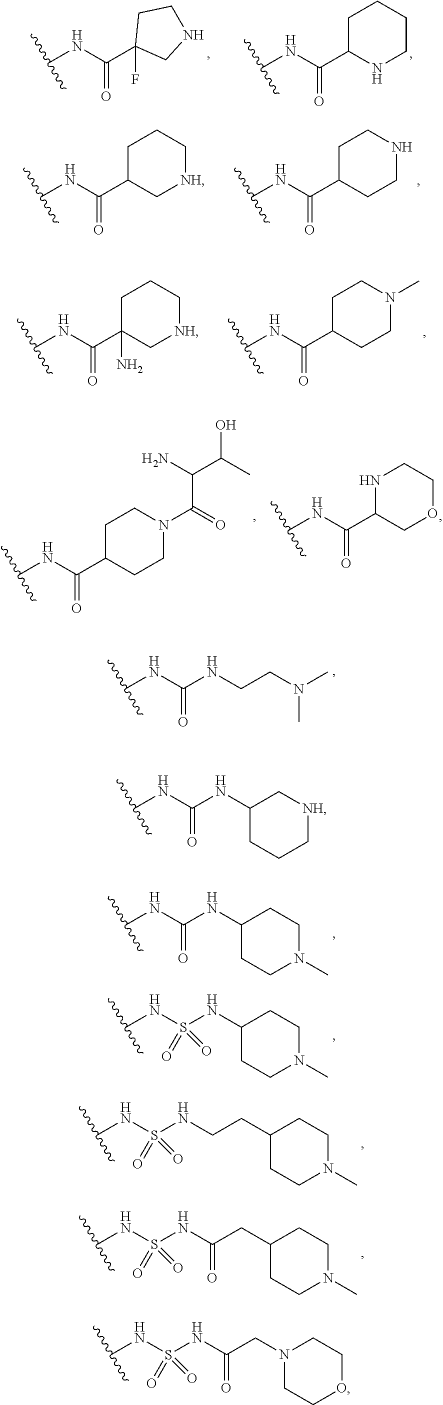

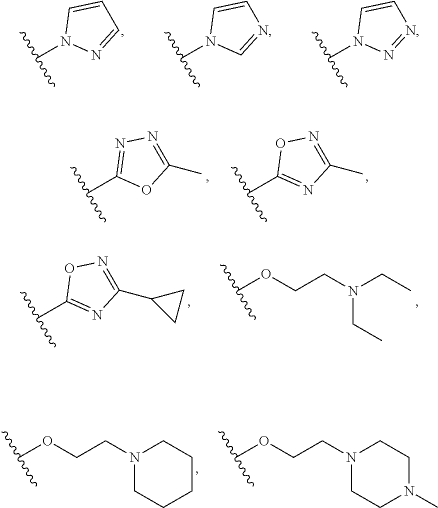

In certain embodiments, each R⁴ is independently

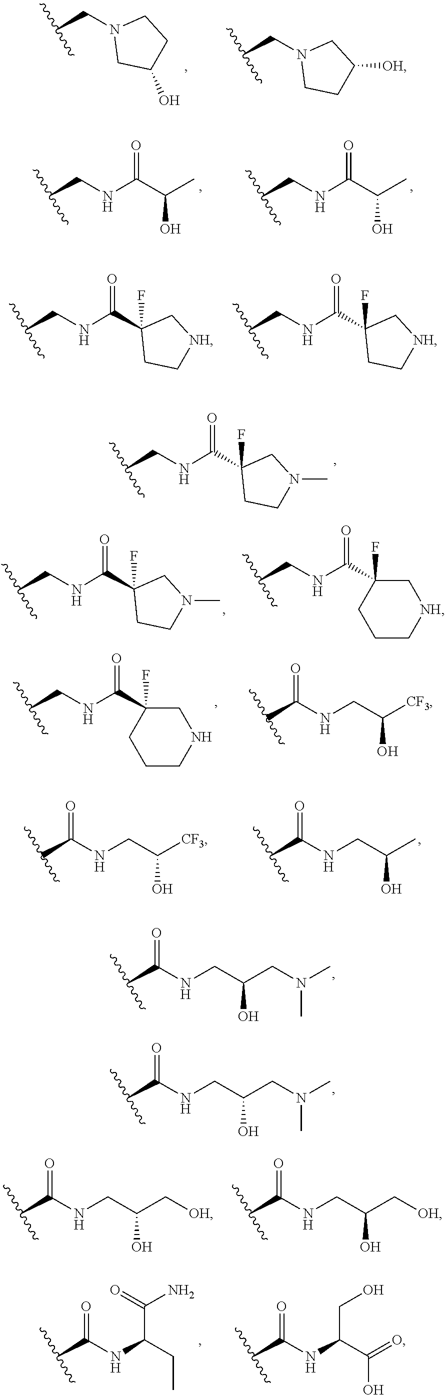

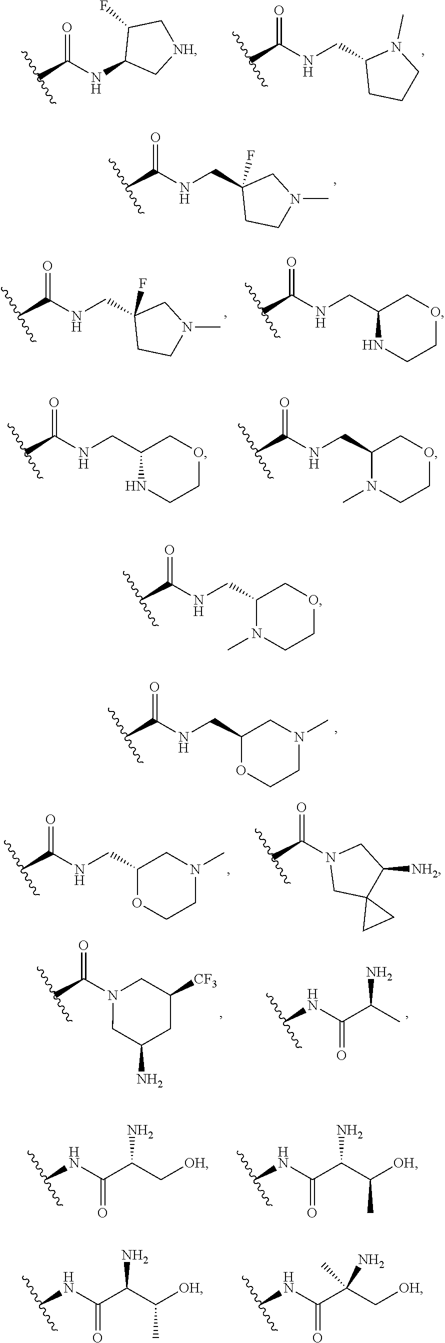

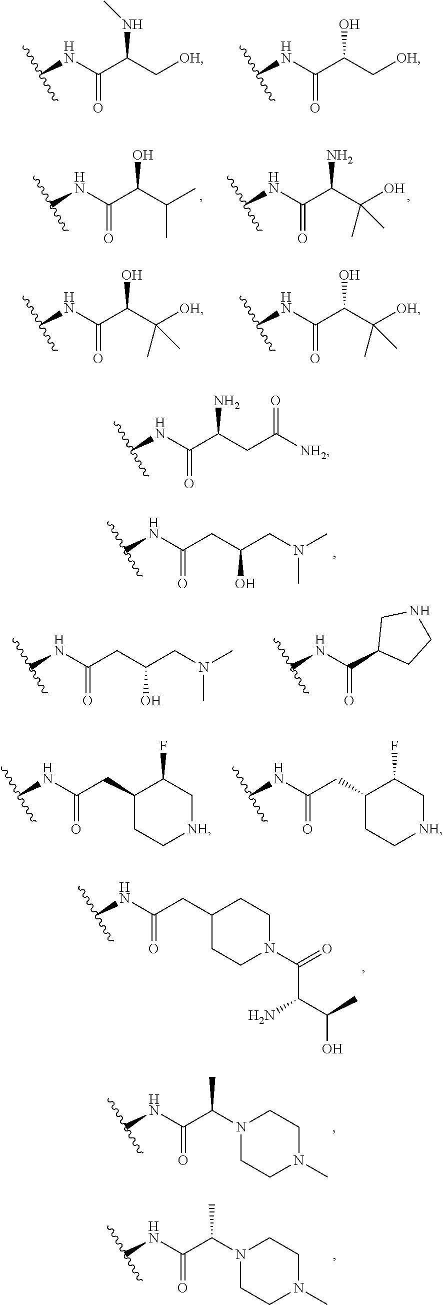

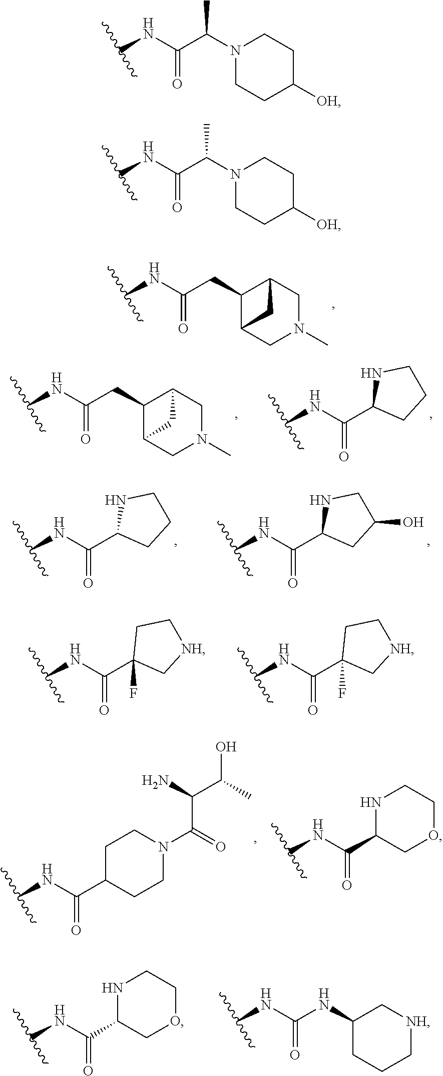

In certain embodiments, each R⁵ is independently —H.

In certain embodiments, each R⁵ is independently C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R⁵ is independently methyl, ethyl, ethyl, propyl, i-propyl, butyl, s-butyl, t-butyl, straight or branched pentyl, or straight or branched hexyl; each of which is optionally substituted.

In certain embodiments, each R⁵ is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R⁵ is independently halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, each R⁵ is independently methyl, cyclopropyl, —F, or —CF₃.

In certain embodiments, each R⁵ is independently

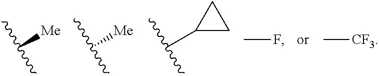

In certain embodiments, k=1. In certain embodiments, r=1. In certain embodiments, t=1. In certain embodiments, n=0. In certain embodiments, p=0. In certain embodiments, both n=0 and p=0. In certain embodiments, r=1 and t=1. In certain embodiments, r=1 and t=1 and k=1. In certain embodiments, r=1 and t=1 and k=1 and n=0 and p=0.

In certain embodiments, each of X, Ring A, Ring B, R¹, R², R³, R⁴, R⁵, k, m, n, p, r, and t, is as defined above and described in embodiments, classes and subclasses above and herein, singly or in combination.

In certain embodiments, the present invention provides a compound of formula I-a,

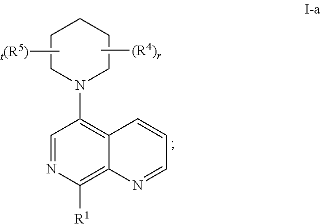

or a pharmaceutically acceptable salt thereof, wherein each of R¹, R⁴, R⁵, r, and t, is as defined above and described in embodiments, classes and subclasses above and herein, singly or in combination.

In certain embodiments, R¹ is -Me, —CF₃, —OMe, or —CN. In certain embodiments, R¹ is —CF₃ or —OMe. In certain embodiments, R¹ is —CF₃. In certain embodiments, R¹ is —OMe.

In certain embodiments, each R⁴ is independently —H, C₁₋₆ aliphatic, —C(O)N(R)₂, —NRC(O)R, or —N(R)₂; each of which is optionally substituted. In certain embodiments, each R⁴ is —N(R)₂. In certain embodiments, each R⁴ is independently

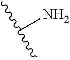 [SMILES: *N]

In certain embodiments, each R⁴ is independently

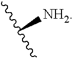

In certain embodiments, each R⁵ is independently methyl, —F, or —CF₃. In certain embodiments, each R⁵ is independently methyl.

In certain embodiments, r=1 and t=1, i.e. embodiments with one substituent R⁴ and one substituent R⁵. In certain embodiments, these single substituents R⁴ and R⁵ have a cis-configuration relative to each other, i.e. their orientation is either

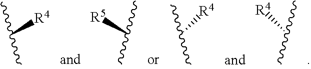

In some embodiments, their orientation is

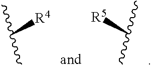

In certain embodiments, the compound of formula I-a is a compound of formula I-aa:

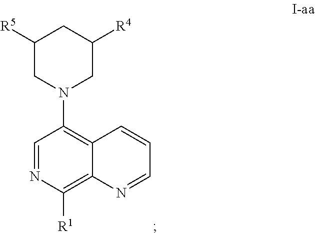

or a pharmaceutically acceptable salt thereof, wherein each of R¹, R⁴, and R⁵, is as defined above and described in embodiments, classes and subclasses above and herein, singly or in combination.

In certain embodiments, R¹ is -Me, —CF₃, —OMe, or —CN. In certain embodiments, R¹ is —CF₃ or —OMe. In certain embodiments, R¹ is —CF₃. In certain embodiments, R¹ is —OMe.

In certain embodiments, R⁴ is C₁₋₆ aliphatic, —C(O)N(R)₂, —NRC(O)R, or —N(R)₂; each of which is optionally substituted. In certain embodiments, R⁴ is —N(R)₂. In certain embodiments, R⁴ is

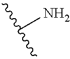 [SMILES: *N]

In certain embodiments, R⁴ is

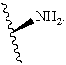

In certain embodiments, R⁵ is methyl, —F, or —CF₃. In certain embodiments, R⁵ is methyl.

In certain embodiments, substituents R⁴ and R⁵ have a cis-configuration relative to each other, i.e. their orientation is either

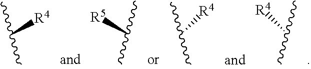

In some embodiments, their orientation is

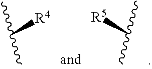

In certain embodiments, the present invention provides a compound of formula I-b,

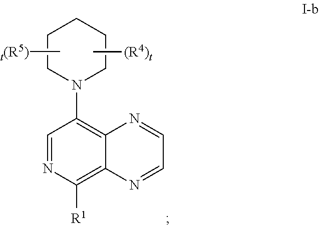

or a pharmaceutically acceptable salt thereof, wherein each of R¹, R⁴, R⁵, r, and t, is as defined above and described in embodiments, classes and subclasses above and herein, singly or in combination.

In certain embodiments, R¹ is -Me, —CF₃, —OMe, or —CN. In certain embodiments, R¹ is —OMe.

In certain embodiments, each R⁴ is independently —H, C₁₋₆ aliphatic, —C(O)N(R)₂, —NRC(O)R, or —N(R)₂; each of which is optionally substituted. In certain embodiments, each R⁴ is —N(R)₂. In certain embodiments, each R⁴ is independently

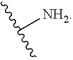 [SMILES: *N]

In certain embodiments, each R⁴ is independently

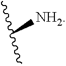

In certain embodiments, each R⁵ is independently methyl, —F, or —CF₃. In certain embodiments, each R⁵ is independently methyl.

In certain embodiments, r=1 and t=1, i.e. embodiments with one substituent R⁴ and one substituent R⁵. In certain embodiments, these single substituents R⁴ and R⁵ have a cis-configuration relative to each other, i.e. their orientation is either

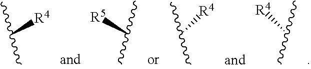

In some embodiments, their orientation is

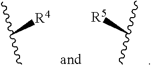

In certain embodiments, the compound of formula I-b is a compound of formula I-ba:

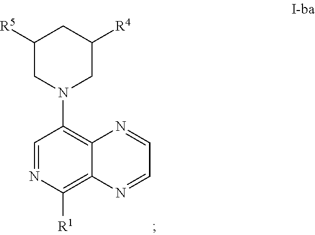

or a pharmaceutically acceptable salt thereof, wherein each of R¹, R⁴, and R⁵ is as defined above and described in embodiments, classes and subclasses above and herein, singly or in combination.

In certain embodiments, R¹ is -Me, —CF₃, —OMe, or —CN. In certain embodiments, R¹ is —OMe.

In certain embodiments, R⁴ is C₁₋₆ aliphatic, —C(O)N(R)₂, —NRC(O)R, or —N(R)₂; each of which is optionally substituted. In certain embodiments, R⁴ is —N(R)₂. In certain embodiments, R⁴ is

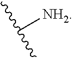 [SMILES: *N]

In certain embodiments, R⁴ is

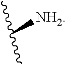

In certain embodiments, R⁵ is methyl, —F, or —CF₃. In certain embodiments, R⁵ is methyl.

In certain embodiments, R⁴ and R⁵ have a cis-configuration relative to each other, i.e. their orientation is either

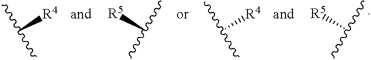

In some embodiments, their orientation is

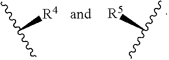

In certain embodiments, the present invention provides a compound of formula I-c,

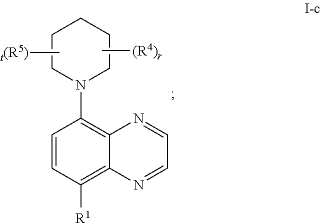

or a pharmaceutically acceptable salt thereof, wherein each of R¹, R⁴, R⁵, r, and t, is as defined above and described in embodiments, classes and subclasses above and herein, singly or in combination.

In certain embodiments, R¹ is -Me, —CF₃, —OMe, or —CN. In certain embodiments, R¹ is —CN.

In certain embodiments, each R⁴ is independently —H, C₁₋₆ aliphatic, —C(O)N(R)₂, —NRC(O)R, or —N(R)₂; each of which is optionally substituted. In certain embodiments, each R⁴ is NRC(O)R, or —N(R)₂. In certain embodiments, each R⁴ is NRC(O)R.

In certain embodiments, each R⁴ is independently

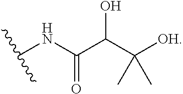 [SMILES: *NC(=O)C(O)C(C)(C)O]

In certain embodiments, each R⁵ is independently methyl, —F, or —CF₃. In certain embodiments, each R⁵ is independently methyl.

In certain embodiments, r=1 and t=1, i.e. embodiments with one substituent R⁴ and one substituent R⁵. In certain embodiments, these single substituents R⁴ and R⁵ have a cis-configuration relative to each other, i.e. their orientation is either

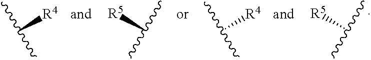

In some embodiments, their orientation is

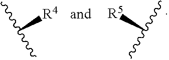

In certain embodiments, the compound of formula I-c is a compound of formula I-ca:

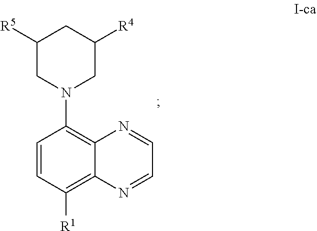

or a pharmaceutically acceptable salt thereof, wherein each of R¹, R⁴, and R⁵ is as defined above and described in embodiments, classes and subclasses above and herein, singly or in combination.

In certain embodiments, R¹ is -Me, —CF₃, —OMe, or —CN. In certain embodiments, R¹ is —CN.

In certain embodiments, R⁴ is C₁₋₆ aliphatic, —C(O)N(R)₂, —NRC(O)R, or —N(R)₂; each of which is optionally substituted. In certain embodiments, R⁴ is NRC(O)R, or —N(R)₂. In certain embodiments, R⁴ is NRC(O)R.

In certain embodiments, R⁴ is independently

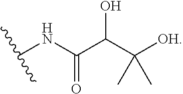 [SMILES: *NC(=O)C(O)C(C)(C)O]

In certain embodiments, R⁵ is methyl, —F, or —CF₃. In certain embodiments, R⁵ is methyl.

In certain embodiments, substituents R⁴ and R⁵ have a cis-configuration relative to each other, i.e. their orientation is either

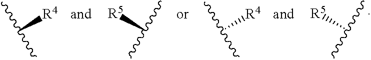

In some embodiments, their orientation is

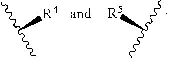

In certain embodiments, the present invention provides a compound of formula I-d,

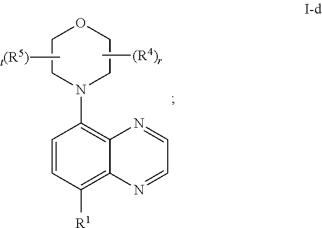

or a pharmaceutically acceptable salt thereof, wherein each of R¹, R⁴, R⁵, r, and t, is as defined above and described in embodiments, classes and subclasses above and herein, singly or in combination.

In certain embodiments, R¹ is -Me, —CF₃, —OMe, or —CN. In certain embodiments, R¹ is —CN.

In certain embodiments, each R⁴ is independently —H, C₁₋₆ aliphatic, —C(O)N(R)₂, —NRC(O)R, or —N(R)₂; each of which is optionally substituted. In certain embodiments, each R⁴ is —C(O)N(R)₂.

In certain embodiments, each R⁴ is independently

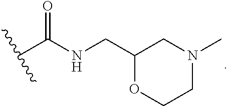 [SMILES: *C(=O)NCC1CN(C)CCO1]

In certain embodiments, each R⁵ is independently methyl, —F, or —CF₃. In certain embodiments, each R⁵ is independently methyl.

In certain embodiments, r=1 and t=1, i.e. embodiments with one substituent R⁴ and one substituent R⁵. In certain embodiments, these single substituents R⁴ and R⁵ have a cis-configuration relative to each other, i.e. their orientation is either

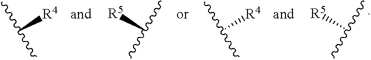

In some embodiments, their orientation is

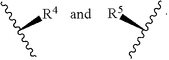

In certain embodiments, the compound of formula I-d is a compound of formula I-da:

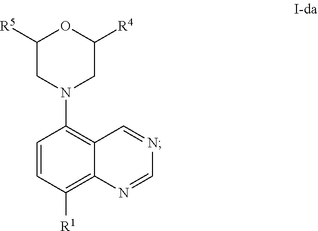

or a pharmaceutically acceptable salt thereof, wherein each of R¹, R⁴, and R⁵ is as defined above and described in embodiments, classes and subclasses above and herein, singly or in combination.

In certain embodiments, R¹ is -Me, —CF₃, —OMe, or —CN. In certain embodiments, R¹ is —CN.

In certain embodiments, R⁴ is C₁₋₆ aliphatic, —C(O)N(R)₂, —NRC(O)R, or —N(R)₂; each of which is optionally substituted. In certain embodiments, R⁴ is —C(O)N(R)₂.

In certain embodiments, R⁴ is

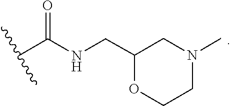 [SMILES: *C(=O)NCC1CN(C)CCO1]

In certain embodiments, R⁵ is methyl, —F, or —CF₃. In certain embodiments, R⁵ is methyl.

In certain embodiments, R⁴ and R⁵ have a cis-configuration relative to each other, i.e. their orientation is either

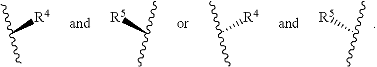

In some embodiments, their orientation is

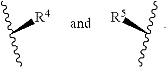

In some embodiments, the present invention provides a compound selected from those depicted above, or a pharmaceutically acceptable salt thereof.

Various structural depictions may show a heteroatom without an attached group, radical, charge, or counterion. Those of ordinary skill in the art are aware that such depictions are meant to indicate that the heteroatom is attached to hydrogen (e.g.,

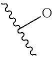 [SMILES: *O]

is understood to be

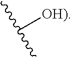

In certain embodiments, the compounds of the invention were synthesized in accordance with the schemes provided in the Examples below.

### 4. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably inhibit TLR7/8, or a mutant thereof, in a biological sample or in a patient. In certain embodiments, the amount of compound in compositions of this invention is such that is effective to measurably inhibit TLR7/8, or a mutant thereof, in a biological sample or in a patient. In certain embodiments, a composition of this invention is formulated for administration to a patient in need of such composition.

The term “patient” or “subject”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that are used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention are administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrastemal, intrathecal, intrahepatic, intralesional and intracranial injection or infusion techniques. Preferably, the compositions are administered orally, intraperitoneally or intravenously. Sterile injectable forms of the compositions of this invention include aqueous or oleaginous suspension. These suspensions are formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that are employed are water, Ringer's solution and isotonic sodium chloride solution. In addition, sterile, fixed oils are conventionally employed as a solvent or suspending medium.

For this purpose, any bland fixed oil employed includes synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms are also be used for the purposes of formulation.

Pharmaceutically acceptable compositions of this invention are orally administered in any orally acceptable dosage form. Exemplary oral dosage forms are capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents are optionally also added.

Alternatively, pharmaceutically acceptable compositions of this invention are administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention are also administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches are also used.

For topical applications, provided pharmaceutically acceptable compositions are formulated in a suitable ointment containing the active component suspended or dissolved in one or more carriers. Exemplary carriers for topical administration of compounds of this are mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions of this invention are optionally administered by nasal aerosol or inhalation. Such compositions are prepared according to techniques well-known in the art of pharmaceutical formulation and are prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that are optionally combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

The present invention furthermore relates to a method for treating a subject suffering from a TLR7/8 related disorder, comprising administering to said subject an effective amount of a compound of formula I and related formulae.

The compounds of the present invention are useful as anticancer agents for cancers that are responsive to TLR7 activation. In certain embodiments, the cancers include, but are not limited to cancer of the breast, bladder, bone, brain, central and peripheral nervous system, colon, endocrine glands, esophagus, endometrium, germ cells, head and neck, kidney, liver, lung, larynx and hypopharynx, mesothelioma, sarcoma, ovary, pancreas, prostate, rectum, renal, small intestine, soft tissue, testis, stomach, skin, ureter, vagina and vulva; inherited cancers, retinomblastoma and Wilms tumor; leukemia, lymphoma, non-Hodgkins disease, chronic and acute myeloid leukaemia, acute lymphoblastic leukemia, Hodgkins disease, multiple myeloma and T-cell lymphoma; myelodysplastic syndrome, plasma cell neoplasia, paraneoplastic syndromes, cancers of unknown primary site and AIDS related malignancies.

In certain embodiments, the compounds of the invention are used to treat cancers of the skin or kidney. Sensitivity of a given cancer to activation of TLR7 can be assessed by, but not limited to measurement of a decrease in primary or metastatic tumor load (minor, partial or complete regression), alterations in the hemogram, altered hormone or cytokine concentrations in the blood, inhibition of further increase of tumor load, stabilization of the disease in the patient, assessment of biomarkers or surrogate markers relevant for the disease, prolonged overall survival of a patient, prolonged time to disease progression of a patient, prolonged progression-free survival of a patient, prolonged disease-free survival of a patient, improved quality of life of a patient, or modulation of the co-morbidity of the disease (for example, but not limited to pain, cachexia, mobilization, hopitalization, altered hemogram, weight loss, wound healing, fever).

The compounds according to the present invention may further be useful as immune response modifiers that can modulate the immune response in a number of different ways, rendering them useful in the treatment of a variety of disorders.

Provided herein are methods of inhibiting an immune response in an individual comprising administering to the individual an effective amount of an inhibitor of TLR7 and/or TLR8 (e.g., TLR inhibitor), using the compounds as described herein. In some variations, the TLR inhibitor inhibits a TLR7-dependent immune response. In some variations, the TLR inhibitor inhibits a TLR8-dependent immune response. In some variations, the TLR inhibitor inhibits a TLR7-dependent and a TLR8-dependent immune response. In some variations, the TLR inhibitor inhibits a TLR7-dependent, a TLR8-dependent, and another TLR-dependent immune response. Unless otherwise noted, the term TLR inhibitor refers to any one of the TLR inhibitors disclosed herein. In some preferred embodiments, the individual is a human patient.

Methods of immunoregulation are provided by the present disclosure and include those that suppress and/or inhibit an immune response, including, but not limited to, an immune response. The present disclosure also provides methods for ameliorating symptoms associated with unwanted immune activation, including, but not limited to, symptoms associated with autoimmunity. Immune suppression and/or inhibition according to the methods described herein may be practiced on individuals including those suffering from a disorder associated with an unwanted activation of an immune response. The present disclosure also provides methods for inhibiting a TLR7 and/or TLR8 induced response (e.g., in vitro or in vivo). In some variations, the cell is contacted with the TLR inhibitor in an amount effective to inhibit a response from the cell that contributes to an immune response.

Inhibition of TLR7 and/or TLR8 are useful for treating and/or preventing a variety of diseases or disorders that are responsive to cytokines. Conditions for which TLR7 and/or TLR8 inhibitors may be used as treatments include, but are not limited to autoimmune diseases and inflammatory disorders. Provided herein are methods of treating or preventing a disease or disorder in an individual comprising administering to the individual an effective amount of an inhibitor of TLR7 and/or TLR8. Further, provided are methods for ameliorating symptoms associated with a disease or disorder comprising administering an effective amount of an inhibitor of TLR7 and/or TLR8 to an individual having the disease or disorder. Methods are also provided herein for preventing or delaying development of a disease or a disorder, comprising administering an effective amount of an inhibitor of one or more of TLR7 and/or TLR8 to an individual having the disease or the disorder. In certain embodiments, the inhibitor is a compound as described herein.

Provided herein are methods of inhibiting an immune response in an individual, the method comprising administering to the individual at least one TLR inhibitor as disclosed herein in an amount effective to inhibit the immune response in the individual. In some variations, the immune response is associated with an autoimmune disease. In further aspects, wherein inhibiting the immune response ameliorates one or more symptoms of the autoimmune disease. In still further aspects, wherein inhibiting the immune response treats the autoimmune disease. In yet further aspects, wherein inhibiting the immune response prevents or delays development of the autoimmune disease. In some variations, the TLR inhibitor inhibits a TLR7-dependent immune response. In some variations, the TLR inhibitor inhibits a TLR8-dependent immune response. In some variations, the TLR inhibitor inhibits a TLR7-dependent and a TLR8-dependent immune response. In some aspects, at least one TLR inhibitor is administered in an amount effective to inhibit an immune response in the individual.

Provided herein are also methods of treating or preventing an autoimmune disease in an individual, comprising administering to the individual an effective amount of a TLR7 and/or TLR8 inhibitor. In some aspects, the autoimmune disease is characterized by joint pain, antinuclear antibody positivity, malar rash, or discoid rash. In some aspects, the autoimmune disease is associated with the skin, muscle tissue, and/or connective tissue. In some embodiments, the autoimmune disease is not evidenced in the individual by skin, muscle tissue, and/or connective tissue symptoms. In some embodiments, the autoimmune disease is systemic. Autoimmune diseases include, without limitation, rheumatoid arthritis (RA), autoimmune pancreatitis (AIP), systemic lupus erythematosus (SLE), type I diabetes mellitus, multiple sclerosis (MS), antiphospholipid syndrome (APS), sclerosing cholangitis, systemic onset arthritis, irritable bowel disease (IBD), scleroderma, Sjogren's disease, vitiligo, polymyositis, pemphigus vulgaris, pemphigus foliaceus, inflammatory bowel disease including Crohn's disease and ulcerative colitis, autoimmune hepatitis, hypopituitarism, graft-versus-host disease (GvHD), autoimmune skin diseases, uveitis, pernicious anemia, and hypoparathyroidism. Autoimmune diseases may also include, without limitation, polyangiitis overlap syndrome, Kawasaki's disease, sarcoidosis, glomerulonephritis, and cryopathies.

In some aspects, the autoimmune disease is selected from the group consisting of arthritis, pancreatitis, mixed connective tissue disease (MCTD), lupus, antiphospholipid syndrome (APS), systemic onset arthritis, and irritable bowel syndrome.

In other aspects, the autoimmune disease is selected from the group consisting of systemic lupus erythematosus (SLE), rheumatoid arthritis, autoimmune skin disease, and multiple sclerosis.

In other aspects, the autoimmune disease is selected from the group consisting of pancreatitis, glomerulonephritis, pyelitis, sclerosing cholangitis, and type I diabetes. In some aspects, the autoimmune disease is rheumatoid arthritis. In some aspects, the autoimmune disease is autoimmune pancreatitis (AIP). In some aspects, the autoimmune disease is glomerulonephritis. In some aspects, the autoimmune disease is pyelitis. In some aspects, the autoimmune disease is sclerosing cholangitis. In some aspects the autoimmune disorder is psoriasis. In some aspects, the autoimmune disease is a rheumatoid disease or disorder. In some aspects, the rheumatoid disease or disorder is rheumatoid arthritis. In some aspects, the disease is diabetes and/or diabetic-related disease or disorder. In some aspects, wherein the autoimmune disease is associated with RNA-containing immune complexes. In some aspects, the autoimmune disease is Sjogren's disease.

Provided herein are methods of inhibiting an immune response in an individual, the method comprising administering to the individual at least one TLR inhibitor as disclosed herein in an amount effective to inhibit the immune response in the individual. In some variations, the immune response is associated with an inflammatory disorder. As used herein, the term “inflammatory disorder” encompasses autoimmune diseases, as well as inflammatory conditions without a known autoimmune component (e.g., artherosclerosis, asthma, etc.). In further aspects, inhibiting the immune response ameliorates one or more symptoms of the inflammatory disorder. In still further aspects, inhibiting the immune response treats the inflammatory disorder. In yet further aspects, inhibiting the immune response prevents or delays development of the inflammatory disorder. In some aspects, the inflammatory disorder is selected from the group consisting of non-rheumatoid arthritis, kidney fibrosis, and liver fibrosis. In some aspects, the inflammatory disorder is an interface dermatitis. In some further aspects, the interface dermatitis is selected from the group consisting of lichen planus, lichenoid eruption, lichen planus-like keratosis, lichen striatus, keratosis lichenoides chronica, erythema multiforme, fixed drug eruption, pityriasis lichenoides, phototoxic dermatitis, radiation dermatitis, viral exanthems, dermatomyositis, secondary syphilis, lichen sclerosus et atrophicus, mycosis fungoides, bullous pemphigoid, lichen aureus, porokeratosis, acrodermatitis chronicus atrophicans, and regressing melanoma. In some aspects, the inflammatory condition is a skin disorder such as atopic dermatitis (eczema). In some aspects, the inflammatory disorder is a sterile inflammatory condition such as drug-induced liver and/or pancreas inflammation. In some further aspects, the inflammatory disease is an inflammatory liver disorder. In some other further aspects, the inflammatory disease is an inflammatory pancreatic disorder.

Provided herein are methods of inhibiting an immune response in an individual, the method comprising administering to the individual at least one TLR inhibitor as disclosed herein in an amount effective to inhibit the immune response in the individual. In some variations, the immune response is associated with chronic pathogen stimulation. In some variations, the immune response is associated with infection by HIV. In further aspects, wherein inhibiting the immune response ameliorates one or more symptoms of the viral disease or disorder resulting from infection by HIV. In still further aspects, wherein inhibiting the immune response treats the viral disease or disorder resulting from infection by HIV. In yet further aspects, wherein inhibiting the immune response prevents or delays development of the viral disease or disorder resulting from infection by HIV. Other variations provided herein relate to immunoinhibitory therapy of individuals having been exposed to or infected with HIV. Administration of a TLR inhibitor to an individual having been exposed to or infected with HIV results in suppression of HIV induced cytokine production. In some aspects, at least one TLR inhibitor is administered in an amount effective to suppress HIV induced cytokine production in an individual exposed to or infected with a HIV.

Provided herein are methods for inhibiting a TLR7 and/or TLR8-dependent immune response in an individual, the method comprising administering to the individual a TLR inhibitor in an amount effective to inhibit the immune response in the individual. In some variations, the immune response is associated with an autoimmune disease. In some aspects, the autoimmune disease is rheumatoid arthritis. In some aspects, the TLR inhibitor is effective in suppressing one or more symptoms of rheumatoid arthritis. In some aspects, the autoimmune disease is multiple sclerosis. In some aspects, the TLR inhibitor is effective in suppressing one or more symptoms of multiple sclerosis. In some aspects, the autoimmune disease is lupus. In some aspects, the TLR inhibitor is effective in suppressing one or more symptoms of lupus. In some aspects, the autoimmune disease is pancreatitis. In some aspects, the TLR inhibitor is effective in suppressing one or more symptoms of pancreatitis. In some aspects, the autoimmune disease is diabetes. In some aspects, the TLR inhibitor is effective in suppressing one or more symptoms of diabetes. In some aspects, the disease is Sjogren's disease. In some aspects, the TLR inhibitor is effective in suppressing one or more symptoms of Sjogren's disease. In some variations, the immune response is associated with an inflammatory disorder. In some aspects, the TLR inhibitor is effective in suppressing one or more symptoms of an inflammatory disorder. In some variations, the immune response is associated with chronic pathogen stimulation. In some aspects, the TLR inhibitor is effective in suppressing one or more symptoms of chronic pathogen stimulation. In some variations, the immune response is associated with viral disease resulting from infection with HIV. In some aspects, the TLR inhibitor is effective in suppressing one or more symptoms of viral disease resulting from infection with HIV. In any variation, the TLR inhibitor is a polynucleotide comprising an inhibitory motif for one or more of TLR7, TLR8, and TLR9.

In some embodiments of any of the methods involving administration of a TLR inhibitor to an individual (e.g., methods of inhibiting an immune response, treating or preventing an autoimmune disease or inflammatory disorder, etc.) the TLR inhibitor has a therapeutically acceptable safety profile. The TLR inhibitor may for example, have a therapeutically acceptable histological profile including an acceptably low, if any, toxicity of the liver, kidney, pancreas, or other organs. On occasion, polynucleotides have been associated with toxicity to certain organs such as the liver, kidney and pancreas. In some embodiments, the TLR inhibitor has a safety profile that is unexpected and advantageous. In some embodiments, a safety profile includes evaluation of toxicity, histological profile, and/or necrosis (e.g., liver, kidneys and/or heart). In some embodiments, the TLR inhibitor has a therapeutically acceptable level of toxicity. In some embodiments, the TLR inhibitor has a reduced level of toxicity as compared to another TLR inhibitor. In some embodiments, the TLR inhibitor induces a therapeutically acceptable reduction in body weight as compared to the initial body weight of a treated individual. In some embodiments, the TLR inhibitor induces less than 5%, 7.5%, 10%, 12.5, or 15% reduction in total body weight. In some embodiments, the TLR inhibitor has a therapeutically acceptable histology profile. In some embodiments, the TLR inhibitor has a better (e.g., lower severity score) histology profile, for example, as compared to a reference TLR inhibitor. In some embodiments, the TLR inhibitor has a better (e.g., lower severity score) histology profile upon evaluation of the liver, kidneys and/or heart, for example. In some embodiments, the TLR inhibitor has a therapeutically acceptable necrosis score. In some embodiments, the TLR inhibitor has reduced necrosis and/or better (e.g., lower) necrosis score, for example, as compared to a reference TLR inhibitor. In some embodiments, the TLR inhibitor has reduced renal and/or hepatocellular necrosis and/or a better renal and/or hepatocellular necrosis score, for example, as compared to a reference TLR inhibitor.

In some embodiments, certain TLR inhibitors of the present invention are non-brain penetrant compounds. These TLR inhibitors may be useful for the prevention and/or treatment of patient's disorders or conditions which do not necessarily require or benefit from penetration of the blood-brain barrier (BBB) by the TLR inhibitor or for which penetration of the BBB may not be desirable.

Accordingly, the invention provides a method of activating TLR7 in an animal, especially a mammal, preferably a human comprising administering an effective amount of a compound of Formula I to the animal. As with all compositions for inhibition of an immune response, the effective amounts and method of administration of the particular TLR inhibitor formulation can vary based on the individual, what condition is to be treated and other factors evident to one skilled in the art. An effective amount of a compound will vary according to factors known in the art but is expected to be a dose of about 0.1 to 10 mg/kg, 0.5 to 10 mg/kg, 1 to 10 mg/kg, 0.1 to 20 mg/kg, 0.1 to 20 mg/kg, or 1 to 20 mg/kg.

The invention also provides a method of treating a viral infection in an animal comprising administering an effective amount of a compound of Formula I to the animal. An amount effective to treat or inhibit a viral infection is an amount that will cause a reduction in one or more of the manifestations of viral infection, such as viral lesions, viral load, rate of virus production, and mortality as compared to untreated control animals. The precise amount will vary according to factors known in the art but is expected to be a dose as indicated above with respect to the activation of TLR7, or a dose of about 100 ng/kg to about 50 mg/kg, preferably about 10 μg/kg to about 5 mg/kg.

In various embodiments, compounds of formula (I), and related formulae exhibit a IC50 for the binding to TLR7/8 of less than about 5 μM, preferably less than about 1 μM and even more preferably less than about 0.100 μM.

The method of the invention can be performed either in-vitro or in-vivo. The susceptibility of a particular cell to treatment with the compounds according to the invention can be particularly determined by in-vitro tests, whether in the course of research or clinical application. Typically, a culture of the cell is combined with a compound according to the invention at various concentrations for a period of time which is sufficient to allow the active agents to inhibit TLR7/8 activity, usually between about one hour and one week. In-vitro treatment can be carried out using cultivated cells from a biopsy sample or cell line.

The host or patient can belong to any mammalian species, for example a primate species, particularly humans; rodents, including mice, rats and hamsters; rabbits; horses, cows, dogs, cats, etc. Animal models are of interest for experimental investigations, providing a model for treatment of human disease.

For identification of a signal transduction pathway and for detection of interactions between various signal transduction pathways, various scientists have developed suitable models or model systems, for example cell culture models and models of transgenic animals. For the determination of certain stages in the signal transduction cascade, interacting compounds can be utilized in order to modulate the signal. The compounds according to the invention can also be used as reagents for testing TLR7/8-dependent signal transduction pathways in animals and/or cell culture models or in the clinical diseases mentioned in this application.

Moreover, the subsequent teaching of the present specification concerning the use of the compounds according to formula (I) and its derivatives for the production of a medicament for the prophylactic or therapeutic treatment and/or monitoring is considered as valid and applicable without restrictions to the use of the compound for the inhibition of TLR7/8 activity if expedient.

The invention also relates to the use of compounds according to formula (I) and/or physiologically acceptable salts thereof for the prophylactic or therapeutic treatment and/or monitoring of diseases that are caused, mediated and/or propagated by TLR7/8 activity. Furthermore, the invention relates to the use of compounds according to formula (I) and/or physiologically acceptable salts thereof for the production of a medicament for the prophylactic or therapeutic treatment and/or monitoring of diseases that are caused, mediated and/or propagated by TLR7/8 activity. In certain embodiments, the invention provides the use of a compound according to formula I or physiologically acceptable salts thereof, for the production of a medicament for the prophylactic or therapeutic treatment of a TLR7/8-mediated disorder.

Compounds of formula (I) and/or a physiologically acceptable salt thereof can furthermore be employed as intermediate for the preparation of further medicament active ingredients. The medicament is preferably prepared in a non-chemical manner, e.g. by combining the active ingredient with at least one solid, fluid and/or semi-fluid carrier or excipient, and optionally in conjunction with a single or more other active substances in an appropriate dosage form.

The compounds of formula (I) according to the invention can be administered before or following an onset of disease once or several times acting as therapy. The aforementioned compounds and medical products of the inventive use are particularly used for the therapeutic treatment. A therapeutically relevant effect relieves to some extent one or more symptoms of a disorder, or returns to normality, either partially or completely, one or more physiological or biochemical parameters associated with or causative of a disease or pathological condition. Monitoring is considered as a kind of treatment provided that the compounds are administered in distinct intervals, e.g. in order to boost the response and eradicate the pathogens and/or symptoms of the disease completely. Either the identical compound or different compounds can be applied. The methods of the invention can also be used to reduce the likelihood of developing a disorder or even prevent the initiation of disorders associated with TLR7/8 activity in advance or to treat the arising and continuing symptoms.

In the meaning of the invention, prophylactic treatment is advisable if the subject possesses any preconditions for the aforementioned physiological or pathological conditions, such as a familial disposition, a genetic defect, or a previously incurred disease.

The invention furthermore relates to a medicament comprising at least one compound according to the invention and/or pharmaceutically usable derivatives, salts, solvates and stereoisomers thereof, including mixtures thereof in all ratios. In certain embodiments, the invention relates to a medicament comprising at least one compound according to the invention and/or physiologically acceptable salts thereof.

A “medicament” in the meaning of the invention is any agent in the field of medicine, which comprises one or more compounds of formula (I) or preparations thereof (e.g. a pharmaceutical composition or pharmaceutical formulation) and can be used in prophylaxis, therapy, follow-up or aftercare of patients who suffer from diseases, which are associated with TLR7/8 activity, in such a way that a pathogenic modification of their overall condition or of the condition of particular regions of the organism could establish at least temporarily.

In various embodiments, the active ingredient may be administered alone or in combination with other treatments. A synergistic effect may be achieved by using more than one compound in the pharmaceutical composition, i.e. the compound of formula (I) is combined with at least another agent as active ingredient, which is either another compound of formula (I) or a compound of different structural scaffold. The active ingredients can be used either simultaneously or sequentially.

The TLR inhibitors of the present disclosure can be administered in combination with one or more additional therapeutic agents. As described herein, the TLR inhibitors can be combined with a physiologically acceptable carrier. The methods described herein may be practiced in combination with other therapies that make up the standard of care for the disorder, such as administration of anti-inflammatory agents.

In some embodiments, a TLR inhibitor as described herein is administered in combination with a corticosteroid. In some embodiments, the corticosteroid is a glucocorticosteroid. In some embodiments, the corticosteroid is a mineralocorticoid. Corticosteroids include, but are not limited to, corticosterone and derivatives, prodrugs, isomers and analogs thereof, cortisone and derivatives, prodrugs, isomers and analogs thereof (i.e., Cortone), aldosterone and derivatives, prodrugs, isomers and analogs thereof, dexamethasone and derivatives, prodrugs, isomers and analogs thereof (i.e., Decadron), prednisone and derivatives, prodrugs, isomers and analogs thereof (i.e., Prelone), fludrocortisones and derivatives, prodrugs, isomers and analogs thereof, hydrocortisone and derivatives, prodrugs, isomers and analogs thereof (i.e., cortisol or Cortef), hydroxycortisone and derivatives, prodrugs, isomers and analogs thereof, betamethasone and derivatives, prodrugs, isomers and analogs thereof (i.e., Celestone), budesonide and derivatives, prodrugs, isomers and analogs thereof (i.e., Entocort EC), methylprednisolone and derivatives, prodrugs, isomers and analogs thereof (i.e., Medrol), prednisolone and derivatives, prodrugs, isomers and analogs thereof (i.e., Deltasone, Crtan, Meticorten, Orasone, or Sterapred), triamcinolone and derivatives, prodrugs, isomers and analogs thereof (i.e., Kenacort or Kenalog), and the like. In some embodiments, the corticosteroid is fludrocortisone or a derivative, prodrug, isomer or analog thereof. In some embodiments, the corticosteroid is fludrocortisone. In some embodiments, the corticosteroid is hydroxycortisone or a derivative, prodrug, isomer or analog thereof. In some embodiments, the corticosteroid is hydroxycortisone.

In some embodiments, the corticosteroid is administered between about any of 0.001 mg to 1 mg, 0.5 mg to 1 mg, 1 mg to 2 mg, 2 mg to 20 mg, 20 mg to 40 mg, 40 to 80 mg, 80 to 120 mg, 120 mg to 200 mg, 200 mg to 500 mg, or 500 mg to 1000 mg per day. In some embodiments, the corticosteroid is administered between about any of 0.1 mg/kg to 0.5 mg/kg, 0.5 mg/kg to 1 mg/kg, 1 mg/kg to 2 mg/kg, 2 mg/kg to 5 mg/kg, 5 mg/kg to 10 mg/kg, 10 mg/kg to 15 mg/kg, 15 mg/kg to 20 mg/kg, 20 mg/kg to 25 mg/kg, 25 mg/kg to 35 mg/kg, or 35 mg/kg to 50 mg/kg per day.

In some embodiments, the TLR inhibitor used in combination therapy, given in amounts of the TLR inhibitor delivered, may be, for example, from about any of 0.1 to 10 mg/kg, 0.5 to 10 mg/kg, 1 to 10 mg/kg, 0.1 to 20 mg/kg, 0.1 to 20 mg/kg, or 1 to 20 mg/kg.

In some embodiments, the TLR inhibitor is administered simultaneously with one or more additional therapeutic agents including, but not limited to, a corticosteroid (simultaneous administration). In some embodiments, the TLR inhibitor is administered sequentially with an additional therapeutic agent including, but not limited to, a corticosteroid (sequential administration). In some embodiments, sequential administration includes administering the TLR inhibitor or additional therapeutic agent followed within about any of one minutes, five minutes, 30 minutes, one hour, five hours, 24 hours, 48 hours, or a week. In some embodiments, the TLR inhibitor is administered by the same route of administration as the additional therapeutic agent. In some embodiments, the TLR inhibitor is administered by a different route of administration than the additional therapeutic agent. In some embodiments, the additional therapeutic agent is administered parentally (e.g., central venous line, intra-arterial, intravenous, intramuscular, intraperitoneal, intradermal, or subcutaneous injection), orally, gastrointestinally, topically, naso-pharyngeal and pulmonary (e.g. inhalation or intranasally). In some embodiments, the additional therapeutic agent is a corticosteroid.

The disclosed compounds of the formula I can be administered in combination with other known therapeutic agents, including anticancer agents. As used here, the term “anticancer agent” relates to any agent which is administered to a patient with cancer for the purposes of treating the cancer.

The anti-cancer treatment defined above may be applied as a monotherapy or may involve, in addition to the herein disclosed compounds of formula I, conventional surgery or radiotherapy or medicinal therapy. Such medicinal therapy, e.g. a chemotherapy or a targeted therapy, may include one or more, but preferably one, of the following anti-tumor agents:

Alkylating agents: such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, TH-302⁴, VAL-083⁴;
Platinum Compounds: such as carboplatin, cisplatin, eptaplatin, miriplatine hydrate, oxaliplatin, lobaplatin, nedaplatin, picoplatin, satraplatin; lobaplatin, nedaplatin, picoplatin, satraplatin;
DNA altering agents: such as amrubicin, bisantrene, decitabine, mitoxantrone, procarbazine, trabectedin, clofarabine; amsacrine, brostallicin, pixantrone, laromustine¹³;
Topoisomerase Inhibitors: such as etoposide, irinotecan, razoxane, sobuzoxane, teniposide, topotecan; amonafide, belotecan, elliptinium acetate, voreloxin;
Microtubule modifiers: such as cabazitaxel, docetaxel, eribulin, ixabepilone, paclitaxel, vinblastine, vincristine, vinorelbine, vindesine, vinflunine; fosbretabulin, tesetaxel;
Antimetabolites: such as asparaginase³, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur; doxifluridine, elacytarabine, raltitrexed, sapacitabine, tegafur²′3, trimetrexate;
Anticancer antibiotics: such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin; aclarubicin, peplomycin, pirarubicin;
Hormones/Antagonists: such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol; acolbifene, danazol, deslorelin, epitiostanol, orteronel, enzalutamide^{1,3};
Aromatase inhibitors: such as aminoglutethimide, anastrozole, exemestane, fadrozole, letrozole, testolactone; formestane;
Small molecule kinase inhibitors: such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifarnib, tivantinib, tivozanib, trametinib, pimasertib, brivanib alaninate, cediranib, apatinib⁴, cabozantinib S-malate^{1,3}, ibrutinibi^{1,3}, icotinib⁴, buparlisib², cipatinib⁴, cobimetinib^{1,3}, idelalisib^{1,3}, fedratinibi, XL-647⁴;
Photosensitizers: such as methoxsalen³; porfimer sodium, talaporfin, temoporfin;
Antibodies: such as alemtuzumab, besilesomab, brentuximab vedotin, cetuximab, denosumab, ipilimumab, ofatumumab, panitumumab, rituximab, tositumomab, trastuzumab, bevacizumab, pertuzumab^{2,3}; catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab^{1,2,3}, onartuzumab^{1,3}, racotumomab¹, tabalumab^{1,3}, EMD-525797⁴, nivolumab^{1,3};
Cytokines: such as aldesleukin, interferon alfa², interferon alfa2a³, interferon alfa2b²′3; celmoleukin, tasonermin, teceleukin, oprelvekin^{1,3}, recombinant interferon beta-1a⁴; Drug Conjugates: such as denileukin diftitox, ibritumomab tiuxetan, iobenguane I123, prednimustine, trastuzumab emtansine, estramustine, gemtuzumab, ozogamicin, aflibercept; cintredekin besudotox, edotreotide, inotuzumab ozogamicin, naptumomab estafenatox, oportuzumab monatox, technetium (99mTc) arcitumomab^{1,3}, vintafolide^{1,3}; Vaccines: such as sipuleucel³; vitespen³, emepepimut-S³, oncoVAX⁴, rindopepimut³, troVax⁴, MGN-16014, MGN-17034; and Miscellaneous: alitretinoin, bexarotene, bortezomib, everolimus, ibandronic acid, imiquimod, lenalidomide, lentinan, metirosine, mifamurtide, pamidronic acid, pegaspargase, pentostatin, sipuleucel³, sizofiran, tamibarotene, temsirolimus, thalidomide, tretinoin, vismodegib, zoledronic acid, vorinostat; celecoxib, cilengitide, entinostat, etanidazole, ganetespib, idronoxil, iniparib, ixazomib, lonidamine, nimorazole, panobinostat, peretinoin, plitidepsin, pomalidomide, procodazol, ridaforolimus, tasquinimod, telotristat, thymalfasin, tirapazamine, tosedostat, trabedersen, ubenimex, valspodar, gendicine⁴, picibanil⁴, reolysin⁴, retaspimycin hydrochloride^{1,3}, trebananib^{2,3}, virulizin⁴, carfilzomib^{1,3}, endostatin⁴, immucothel⁴, belinostat³, MGN-17034.
(¹ Prop. INN (Proposed International Nonproprietary Name); ²Rec. INN (Recommended International Nonproprietary Names); ³USAN (United States Adopted Name); ⁴no INN).

In some embodiments, the combination of a TLR inhibitor with one or more additional therapeutic agents reduces the effective amount (including, but not limited to, dosage volume, dosage concentration, and/or total drug dose administered) of the TLR inhibitor and/or the one or more additional therapeutic agents administered to achieve the same result as compared to the effective amount administered when the TLR inhibitor or the additional therapeutic agent is administered alone. In some embodiments, the combination of a TLR inhibitor with a corticosteroid reduces the effective amount of corticosteroid administered as compared to the corticosteroid administered alone. In some embodiments, the combination of a TLR inhibitor with the additional therapeutic agents reduces the frequency of administrations of the therapeutic agent compared to administration of the additional therapeutic agent alone. In some embodiments, the combination of a TLR inhibitor with the additional therapeutic agent reduces the total duration of treatment compared to administration of the additional therapeutic agent alone. In some embodiments, the combination of a TLR inhibitor with the additional therapeutic agent reduces the side effects associated with administration of the additional therapeutic agent alone. In some embodiments, the additional therapeutic agent is a corticosteroid. In some embodiments, the corticosteroid is fludrocortisone or a derivative, prodrug, isomer or analog thereof. In some embodiments, the corticosteroid is fludrocortisone. In some embodiments, the combination of an effective amount of the TLR inhibitor with the additional therapeutic agent is more efficacious compared to an effective amount of the TLR inhibitor or the additional therapeutic agent alone.

TLR inhibitors also may be useful as a vaccine adjuvant for use in conjunction with any material that modulates either humoral and/or cell mediated immune response, such as, for example, live viral, bacterial, or parasitic immunogens; inactivated viral, tumor-derived, protozoal, organism-derived, fungal, or bacterial immunogens, toxoids, toxins; self-antigens; polysaccharides; proteins; glycoproteins; peptides; cellular vaccines; DNA vaccines; recombinant proteins; glycoproteins; peptides; and the like. In some aspects, the combination therapy including but not limited to the combination of a TLR inhibitor and a vaccine is used in the treatment of an autoimmune disease or an inflammatory disorder. In some aspects, the combination therapy including but not limited to the combination of a TLR inhibitor and a vaccine is used in the treatment of an infectious disease.

In some embodiments, the combination therapy including but not limited to the combination of a TLR inhibitor and a corticosteroid is used in the treatment of an autoimmune disease or an inflammatory disorder. In some embodiments, the autoimmune disease is selected from but not limited to rheumatoid arthritis, systemic lupus erythematosus, autoimmune skin disease, multiple sclerosis, pancreatitis, glomerulonephritis, pyelitis, Sclerosing cholangitis, and type I diabetes. In some embodiments, the autoimmune disease is Sjogren's disease.

Also provided herein are kits comprising a TLR inhibitor as provided herein, and instructions for use in the methods of inhibiting a TLR7- and/or TLR8-dependent immune response.

The kits may comprise one or more containers comprising a TLR inhibitor (or a formulation comprising a TLR inhibitor) as described herein, and a set of instructions, generally written instructions although electronic storage media (e.g., magnetic diskette or optical disk) containing instructions are also acceptable, relating to the use and dosage of the TLR inhibitor or formulation for the intended treatment (e.g., suppression of a response to a TLR7 and/or TLR8 agonists, suppression of a TLR7 and/or TLR8-dependent immune response, ameliorating one or more symptoms of an autoimmune disease, ameliorating a symptom of chronic inflammatory disease, decreasing cytokine production in response to a virus, and/or treating and/or preventing one or more symptoms of a disease or disorder mediated by TLR7 and/or TLR8). The instructions included with the kit generally include information as to dosage, dosing schedule, and route of administration for the intended treatment. The containers for the TLR inhibitor (or formulations comprising a TLR inhibitor) may be unit doses, bulk packages (e.g., multi-dose packages) or sub-unit doses. The kits may further comprise a container comprising an adjuvant.

In another aspect, the invention provides for a kit consisting of separate packs of an effective amount of a compound according to the invention and/or pharmaceutically acceptable salts, derivatives, solvates and stereoisomers thereof, including mixtures thereof in all ratios, and optionally, an effective amount of a further active ingredient. The kit comprises suitable containers, such as boxes, individual bottles, bags or ampoules. The kit may, for example, comprise separate ampoules, each containing an effective amount of a compound according to the invention and/or pharmaceutically acceptable salts, derivatives, solvates and stereoisomers thereof, including mixtures thereof in all ratios, and an effective amount of a further active ingredient in dissolved or lyophilized form.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent or delay their recurrence.

The compounds and compositions, according to the method of the present invention, are administered using any amount and any route of administration effective for treating or lessening the severity of a disorder provided above. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention are administered orally or parenterally at dosage levels of about 0.01 mg/kg to about 100 mg/kg and preferably from about 1 mg/kg to about 50 mg/kg, of subject body weight per day, one or more times a day, to obtain the desired therapeutic effect.

In certain embodiments, a therapeutically effective amount of a compound of the formula (I), and related formulae and of the other active ingredient depends on a number of factors, including, for example, the age and weight of the animal, the precise disease condition which requires treatment, and its severity, the nature of the formulation and the method of administration, and is ultimately determined by the treating doctor or vet. However, an effective amount of a compound is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound per se.

In certain embodiments, the pharmaceutical formulations can be administered in the form of dosage units, which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the disease condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process, which is generally known in the pharmaceutical art.

Liquid dosage forms for oral administration include, but are not limited to, pharmaceutically acceptable emulsions, microemulsions, solutions, suspensions, syrups and elixirs. In addition to the active compounds, the liquid dosage forms optionally contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions are formulated according to the known art using suitable dispersing or wetting agents and suspending agents. The sterile injectable preparation are also a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This is accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form also optionally comprises buffering agents.

Solid compositions of a similar type are also employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type are also employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms also comprise, as is normal practice, additional substances other than inert diluents, e.g., tableting lubricants and other tableting aids such a magnesium stearate and microcrystalline cellulose. In the case of capsules, tablets and pills, the dosage forms optionally also comprise buffering agents. They optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting TLR7/8 activity in a biological sample comprising the step of contacting said biological sample with a compound of this invention, or a composition comprising said compound.

According to another embodiment, the invention relates to a method of inhibiting TLR7/8, or a mutant thereof, activity in a biological sample in a positive manner, comprising the step of contacting said biological sample with a compound of this invention, or a composition comprising said compound.

The compounds of the invention are useful in-vitro as unique tools for understanding the biological role of TLR7/8, including the evaluation of the many factors thought to influence, and be influenced by, the production of TLR7/8 and the interaction of TLR7/8. The present compounds are also useful in the development of other compounds that interact with TLR7/8 since the present compounds provide important structure-activity relationship (SAR) information that facilitate that development. Compounds of the present invention that bind to TLR7/8 can be used as reagents for detecting TLR7/8 in living cells, fixed cells, in biological fluids, in tissue homogenates, in purified, natural biological materials, etc. For example, by labeling such compounds, one can identify cells expressing TLR7/8. In addition, based on their ability to bind TLR7/8, compounds of the present invention can be used in in-situ staining, FACS (fluorescence-activated cell sorting), sodium dodecyl sulfate polyacrylamide gel electrophoresis (SDS-PAGE), ELISA (enzyme-linked immunoadsorptive assay), etc., enzyme purification, or in purifying cells expressing TLR7/8 inside permeabilized cells. The compounds of the invention can also be utilized as commercial research reagents for various medical research and diagnostic uses. Such uses can include but are not limited to: use as a calibration standard for quantifying the activities of candidate TLR7/8 inhibitors in a variety of functional assays; use as blocking reagents in random compound screening, i.e. in looking for new families of TLR7/8 ligands, the compounds can be used to block recovery of the presently claimed TLR7/8 compounds; use in the co-crystallization with TLR7/8, i.e. the compounds of the present invention will allow formation of crystals of the compound bound to TLR7/8, enabling the determination of enzyme/compound structure by x-ray crystallography; other research and diagnostic applications, wherein TLR7/8 is preferably activated or such activation is conveniently calibrated against a known quantity of an TLR7/8 inhibitor, etc.; use in assays as probes for determining the expression of TLR7/8 in cells; and developing assays for detecting compounds which bind to the same site as the TLR7/8 binding ligands.

The compounds of the invention can be applied either themselves and/or in combination with physical measurements for diagnostics of treatment effectiveness. Pharmaceutical compositions containing said compounds and the use of said compounds to treat TLR7/8-mediated conditions is a promising, novel approach for a broad spectrum of therapies causing a direct and immediate improvement in the state of health, whether in human or in animal. The orally bioavailable and active new chemical entities of the invention improve convenience for patients and compliance for physicians.

The compounds of formula (I), their salts, isomers, tautomers, enantiomeric forms, diastereomers, racemates, derivatives, prodrugs and/or metabolites are characterized by a high specificity and stability, low manufacturing costs and convenient handling. These features form the basis for a reproducible action, wherein the lack of cross-reactivity is included, and for a reliable and safe interaction with the target structure.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Modulation of TLR7/8, or a mutant thereof, activity in a biological sample is useful for a variety of purposes that are known to one of skill in the art. Examples of such purposes include, but are not limited to, blood transfusion, organ transplantation, biological specimen storage, and biological assays.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

The symbols and conventions used in the following descriptions of processes, schemes, and examples are consistent with those used in the contemporary scientific literature, for example, the Journal of the American Chemical Society or the Journal of Biological Chemistry.

Unless otherwise indicated, all temperatures are expressed in ° C. (degrees Centigrade).

All solvents used were commercially available and were used without further purification. Reactions were typically run using anhydrous solvents under an inert atmosphere of nitrogen. Flash column chromatography was generally carried out using Silica gel 60 (0.035-0.070 mm particle size).

All NMR experiments were recorded either on Bruker Mercury Plus 400 NMR Spectrometer equipped with a Bruker 400 BBFO probe at 400 MHz for proton NMR or on Bruker Mercury Plus 300 NMR Spectrometer equipped with a Bruker 300 BBFO probe at 300 MHz for proton NMR. All deuterated solvents contained typically 0.03% to 0.05% v/v tetramethylsilane, which was used as the reference signal (set at δ 0.00 for both ¹H and ¹³C).

LC-MS analyses were performed on a SHIMADZU LC-MS machine consisting of an UFLC 20-AD system and LCMS 2020 MS detector. The column used was a Shim-pack XR-ODS, 2.2 μm, 3.0×50 mm. A linear gradient was applied, starting at 95% A (A: 0.05% TFA in water) and ending at 100% B (B: 0.05% TFA in acetonitrile) over 2.2 min with a total run time of 3.6 min. The column temperature was at 40° C. with the flow rate at 1.0 mL/min. The Diode Array detector was scanned from 200-400 nm. The mass spectrometer was equipped with an electro spray ion source (ES) operated in a positive or negative mode. The mass spectrometer was scanned between m/z 90-900 with a scan time of 0.6 s.

In general, the compounds according to Formula (I) and related formulae of this invention can be prepared from readily available starting materials. If such starting materials are not commercially available, they may be prepared by standard synthetic techniques. In general, the synthesis pathways for any individual compound of Formula (I) and related formulae will depend on the specific substituents of each molecule, such factors being appreciated by those of ordinary skilled in the art. The following general methods and procedures described hereinafter in the examples may be employed to prepare compounds of Formula (I) and related formulae. Reaction conditions depicted in the following schemes, such as temperatures, solvents, or co-reagents, are given as examples only and are not restrictive. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures. For all the protection and deprotection methods, see Philip J. Kocienski, in “Protecting Groups”, Georg Thieme Verlag Stuttgart, N.Y., 1994 and, Theodora W. Greene and Peter G. M. Wuts in “Protective Groups in Organic Synthesis”, Wiley Interscience, 3^{rd} Edition 1999.

### Preparation of Intermediates

### Intermediate 1: 8-[cis-3-hydroxy-5-methylpiperidin-1-yl]quinoxaline-5-carbonitrile

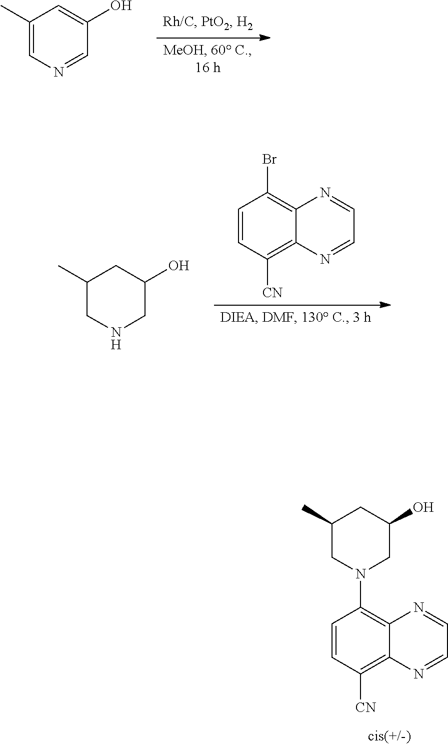

5-methylpiperidin-3-ol: At room temperature, 5-methylpyridin-3-ol (9.50 g, 87.06 mmol), PtO₂ (2767 mg, 12.19 mmol) and Rh/C (2866 mg, 27.86 mmol) were added to a 500 mL pressure tank, followed by the addition of AcOH (200 mL). The tank was vacuumed and flushed with hydrogen. The reaction mixture was hydrogenated for 16 h at 60° C. under 30 atm hydrogen atmosphere. When the reaction was done, the reaction mixture was filtered through a Celite pad and the filtrate was concentrated under reduced pressure to yield the title compound as brown oil (6.80 g, 68%). MS: 116 [M+H]⁺.

8-[cis-3-hydroxy-5-methylpiperidin-1-yl]quinoxaline-5-carbonitrile: To a solution of 8-bromoquinoxaline-5-carbonitrile (450 mg, 1.92 mmol) in DMF (15 mL) was added 5-methylpiperidin-3-ol (246 mg, 2.13 mmol) and DIEA (593 mg, 4.60 mmol) at room temperature. The resulting mixture was stirred for 3 h at 130° C. After cooling to room temperature, the reaction mixture was quenched by the addition of water (50 mL). The resulting mixture was extracted with dichloromethane (100 mL×3). The organic phases were combined, washed with brine and dried over Na₂SO₄. The solvent was removed under reduced pressure and the residue was purified by flash chromatography eluting with ethyl acetate in hexane (0% to 60% gradient) to separate cis/trans isomers and yield 8-[cis-3-hydroxy-5-methylpiperidin-1-yl]quinoxaline-5-carbonitrile as a yellow solid (270 mg, 52%). MS: 269 [M+H]⁺.

### Intermediate 2: cis-5-methyl-1-[8-(trifluoromethyl)quinolin-5-yl]piperidin-3-ol

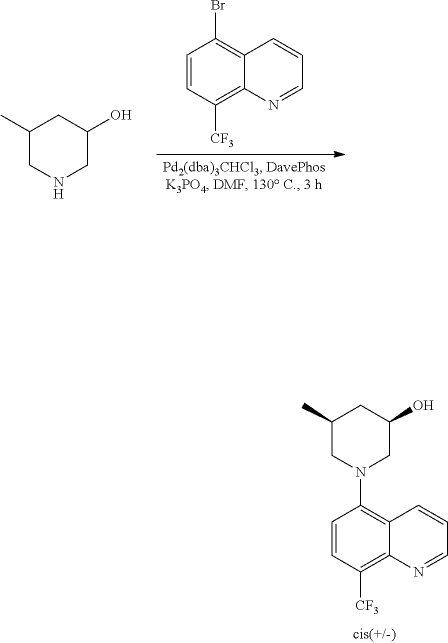

To a solution of 5-bromo-8-(trifluoromethyl)quinoline (950 mg, 3.44 mmol) in DMF (10 mL) was added 5-methylpiperidin-3-ol (600 mg, 5.21 mmol), K₃PO₄ (4161 mg, 19.60 mmol), Pd₂(dba)₃CHCl₃ (676 mg, 0.65 mmol), DavePhos (518 mg, 1.32 mmol) at room temperature under nitrogen atmosphere. The resulting mixture was stirred for 3 h at 130° C. under nitrogen atmosphere. When the reaction was done, it was quenched by the addition of water (20 mL). The resulting mixture was extracted with ethyl acetate (50 mL×3). The organic phases were combined, washed with brine and dried over Na₂SO₄. The solvent was removed under reduced pressure and the residue was purified by reverse phase flash chromatography eluting with acetonitrile in water (5% to 90% gradient in 40 min) to yield cis-5-methyl-1-[8-(trifluoromethyl)quinolin-5-yl]piperidin-3-ol as a yellow solid (638 mg, 60%). MS: 311 [M+H]⁺.

### Intermediate 3: 5-Bromo-7-fluoro-8-methyl-quinoline

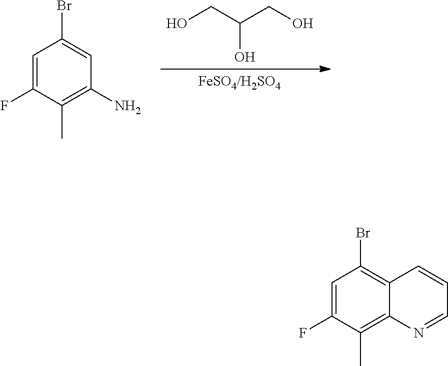

To 5-Bromo-3-fluoro-2-methyl-phenylamine (10.0 g; 49.01 mmol) in 200 ml flask was added glycerol (14.44 ml; 196.04 mmol), iron(ii) sulfate heptahydrate (2.73 g; 9.80 mmol), and sulfuric acid (16 ml; 294.06 mmol). The mixture was stirred at 125° C. for 4 hr. The completed reaction was cooled to room temperature and diluted with 200 ml of DCM. 2N Sodium hydroxide (269 ml; 539.11 mmol) was added slowly to the mixture cooled with ice bath, followed by another 100 ml of DCM. The mixture was stirred for 30 mins at rt. The separated organic layer was washed with brine, dried and concentrated. The crude brown oil was purified by Biotage silica gel column (340 g, eluted with EA/Hexane 10-35%) to yield the title compound as white solid (6.0 g, yield 51%). MS: 241 [M+H]⁺.

### Intermediate 4: 5-Bromo-7-fluoro-quinoline-8-carbonitrile

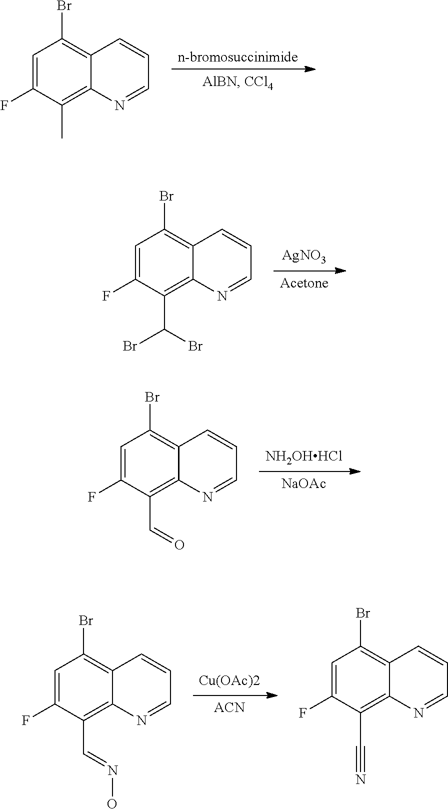

5-Bromo-8-dibromomethyl-7-fluoro-quinoline: To 5-Bromo-7-fluoro-8-methyl-quinoline (2000 mg; 8.33 mmol) and N-bromosuccinimide (3744 mg; 20.83 mmol) was added 60 ml of CCl₄, followed by 2,2′-azobis(2-methylpropionitrile) (205 mg; 1.25 mmol). The mixture was stirred at 80° C. overnight. The reaction mixture was cooled to rt and filtered to remove the solid. The filtrate was concentrated to yield the title compound as a white solid (2800 mg, yield 84.5%). MS: 397/399 [M+H]⁺.

5-Bromo-7-fluoro-quinoline-8-carbaldehyde: To a stirred solution of 5-Bromo-8-dibromomethyl-7-fluoro-quinoline (11.0 g; 27.65 mmol) in acetone (200 ml) and water (40 ml) was added AgNO₃ (11.74 g; 69.12 mmol) at RT. The mixture was stirred at RT for 15 min. The precipitate was removed by filtration and washed with DCM (100 ml). The filtrate was concentrated to 1/3 volume and then extracted with DCM (100 ml×2). The combined organic phases were concentrated to yield the title compound as a yellow solid (7.0 g, 99%), which was directly used for the next step reaction. MS: 255 [M+H]⁺.

5-Bromo-7-fluoro-quinoline-8-carbaldehyde oxime: To 5-Bromo-7-fluoro-quinoline-8-carbaldehyde (7.0 g; 27.55 mmol) in ethanol (300 ml) was added NaOAc (4.52 g; 55.11 mmol) followed by NH₂OH.HCl (2.30 g; 33.06 mmol). The mixture was stirred at 70° C. for 2 hr. The completed reaction was cooled, filtered and washed with ethanol to remove solid. The filtrate was concentrated to yield the title compound as a light yellow solid (7.2 g, yield 97%), which was directly used for the next step reaction. MS: 270 [M+H]⁺.

5-Bromo-7-fluoro-quinoline-8-carbonitrile: To 5-Bromo-7-fluoro-quinoline-8-carbaldehyde oxime (6.0 g; 22.30 mmol) in ACN (20 ml) was added Cu(OAc)₂ (1.01 g; 5.57 mmol) and CH₃COOH (1.28 ml; 22.30 mmol). The mixture was refluxed for 2 hr. LCMS showed the formation of the desired product (˜60%) and by-product. The reaction mixture was cooled and concentrated. The residue was dissolved in 100 ml of EA and 30 ml of 5% aq. NaHCO₃. The separated aqueous layer was extracted with 50 ml of EA. The combined organic layers were washed with brine, dried, and concentrated. The crude was purified with Biotage silica gel column (200 g, eluting with EA/hexane 0-60%) to yield the title compound (1230 mg, yield 22%). MS: 252 [M+H]⁺.

### Intermediate 5: 5-bromo-1,7-naphthyridine-8-carbonitrile

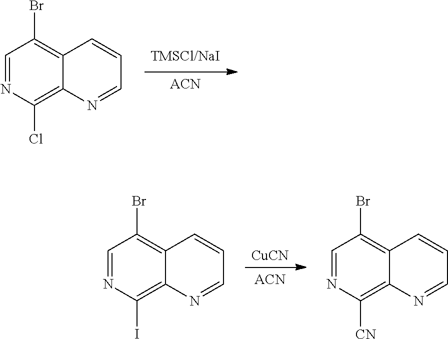

5-Bromo-8-iodo-[1,7] naphthyridine: To a solution of 5-bromo-8-chloro-1,7-naphthyridine (4581 mg; 18.81 mmol; 1.0 eq.), sodium iodide (8.46 g; 56.44 mmol; 3.0 eq.) in 10 ml of ACN was added TMSCl (2.39 ml; 18.81 mmol; 1.0 eq.). The suspension was heated to reflux for 2 h. The tan suspension was cooled to room temperature, poured into water (70 mL), and the brown suspension was stirred at room temperature for 1 h. The beige solid was filtered, washed with water, then dried under vacuo to offer the title compound in quantitively yield. MS:335 [M+H]⁺.

5-bromo-1,7-naphthyridine-8-carbonitrile: To a microwave vial with 5-bromo-8-iodo-[1,7]naphthyridine (3.07 g; 9.17 mmol; 1.0 eq.) was added copper(i) cyanide (0.99 g; 11.0 mmol; 1.20 eq.), and MeCN (8.0 ml). The mixture was stirred at 90° C. in microwave for 1 h. The mixture was diluted with EtOAc (50 mL), and filtered, concentrated, and the residue was used directly for next step. MS: 234 [M+H]⁺.

### Intermediate 6: 5-Bromo-8-trifluoromethyl-[1,7] naphthyridine

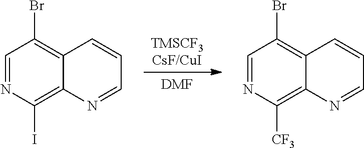

A solution of 5-Bromo-8-iodo-[1,7]naphthyridine (1200 mg; 3.58 mmol; 1.0 eq.), cesium fluoride (1088 mg; 7.17 mmol; 2.0 eq.) and copper iodide (1365 mg, 7.17 mmol, 2 eq) in DMF (10 ml) was added Trimethyl-trifluoromethyl-silane (2.0M in THF) (3.58 ml; 7.17 mmol; 2.0 eq.), the mixture was stirred at RT for 2 h until the reaction was completed. The reaction was diluted with EA, filtered through ceilite, the filtrate was concentrated and the residue was subjected to silica column for purification (eluted with 0-50% EA/hexane) to yield the title compound as a white solid (900 mg, yield 90.7%). LC-MS (M+1)=278/280.

### Intermediate 7: 8-Bromo-pyrido[3,4-b]pyrazine-5-carbonitrile

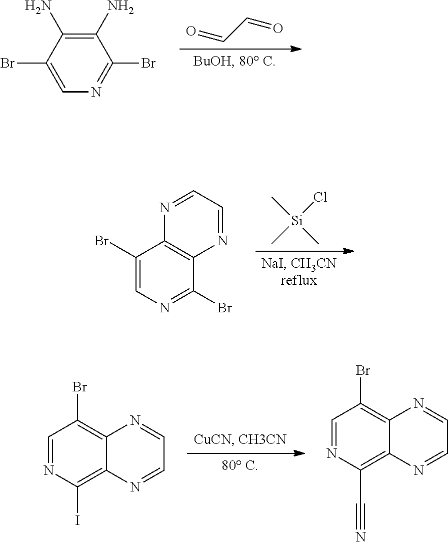

5,8-Dibromo-pyrido[3,4-b]pyrazine: In a 100 mL round bottom flask, 2,5-dibromopyridine-3,4-diamine (2.0 g; 7.493 mmol) was suspended in 1-butanol (50.0 ml) and added a 40% solution of glyoxal (2.1 ml; 18.7 mmol) in water. The tan suspension was heated to 80° C. and the yellow solution was stirred at 80° C. for 1 h 30 min. The orange solution was cooled to room temperature. The beige suspension was filtered, the beige solid was washed with water and hexanes and dried under vacuo to obtain 1.32 g of 5,8-Dibromo-pyrido[3,4-b]pyrazine (1.32 g; 59.1%). MS:290 [M+H]⁺.

8-Bromo-5-iodo-pyrido[3,4-b]pyrazine: In a 50 mL round bottom flask, fitted with a condenser and under nitrogen, 5,8-Dibromo-pyrido[3,4-b]pyrazine (750.0 mg; 2.518 mmol), sodium iodide (1.1 g; 7.554 mmol) and chlorotrimethylsilane (319.6 μl; 2.518 mmol) were added to anhydrous MeCN (5.0 ml). The brown suspension was heated to reflux and the tan suspension was stirred at reflux for 2 h. The tan suspension was let cool to room temperature, poured into water (70 mL) and the brown suspension was stirred at room temperature for 30 min. The beige solid was filtered and the solid was dissolved in DCM and MeOH, adsorbed on a PuriFlash 10 g celite column and purified by chromatography on a PuriFlash 40 g 30u column (DCM for 20 column volumes). The major product eluted between 0.9 to 3.9 column volumes. The pure fractions were concentrated under reduced pressure and the brown solid was dried under vacuo to give 492 mg of a brown solid as the title compound (492.0 mg; 56.1%). MS:336 [M+H]⁺.

8-Bromo-pyrido[3,4-b]pyrazine-5-carbonitrile: In a 10 mL microwave vial, under nitrogen, 8-Bromo-5-iodo-pyrido[3,4-b]pyrazine (200.0 mg; 0.575 mmol) and copper(i) cyanide (61.7 mg; 0.689 mmol) were suspended in anhydrous MeCN (5.0 ml). The tube was sealed and flushed with nitrogen for 10 min and the tan suspension was microwaved at 80° C. for 8 hours. The reaction mixture was concentrated under reduced pressure, the residue was suspended in DCM, filtered on celite and concentrated under reduced pressure. The residue was suspended in DCM, absorbed on a PuriFlash celite 2 g column and purified by chromatography on a PuriFlash 12 g 30u column (Hexanes-AcOEt 20% for 5 column volumes, Hexanes-AcOEt 20-80% for 15 column volumes). The major product was eluted with AcOEt 20-39% (lambda max 245 nm). The pure fractions were concentrated under reduced pressure and the off-white solid was dried under vacuo to give 84 mg of a cream solid as the title compound (84.0 mg; 54.5%). MS:235 [M+H]⁺.

### Intermediate 8: 8-Bromo-5-methoxy-pyrido[3,4-b]pyrazine

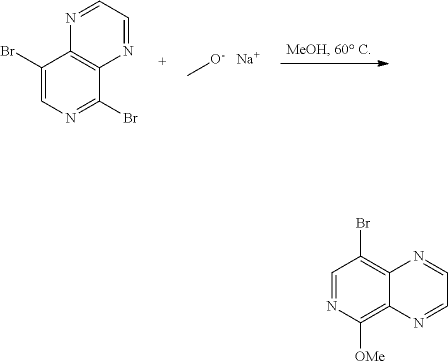

In a 100 mL round bottom flask, under nitrogen, 5,8-Dibromo-pyrido[3,4-b]pyrazine (500.0 mg; 1.731 mmol) was dissolved in anhydrous methanol (50.0 ml). A 0.5M solution of sodium methoxide (5.2 ml; 2.596 mmol) in methanol was added to the beige solution. The beige suspension was heated to 60° C. and the tan solution was stirred at 60° C. for 30 min. The tan solution was cooled to room temperature, quenched with water (10 mL) and concentrated under reduced pressure. The residue was suspended in water (50 mL). The beige suspension was stirred at room temperature for 30 min. The beige solid was filtered, washed with water and dried under vacuo to obtain 331 mg of an beige solid as the title compound (331.0 mg; 79.7%). MS:240 [M+H]⁺.

### Intermediate 9: [cis-6-(trifluoromethyl)morpholin-2-yl]methanol

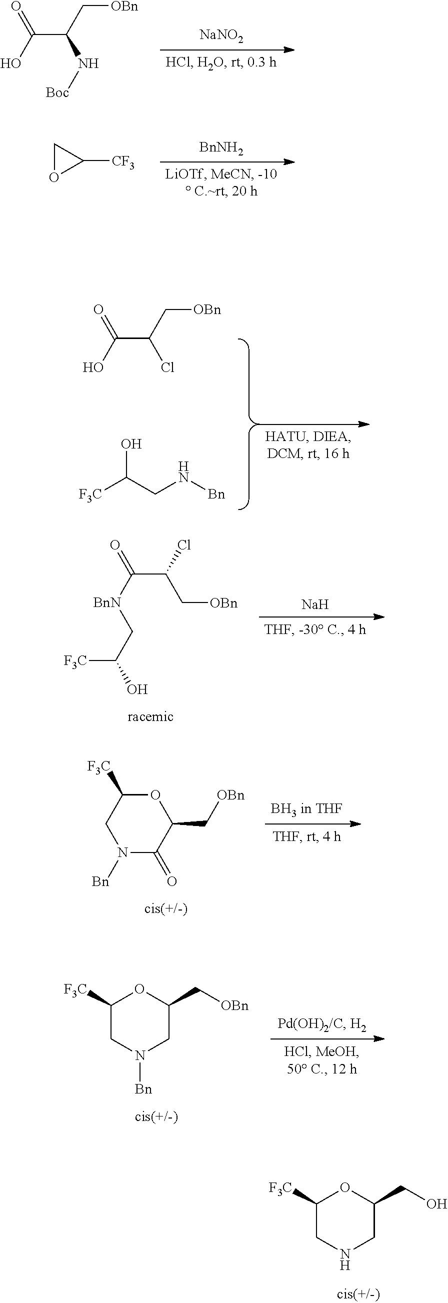

3-(benzyloxy)-2-chloropropanoic acid: At 0° C., to a solution of (2R)-3-(benzyloxy)-2-[[(tert-butoxy)carbonyl]amino]propanoic acid (17.0 g, 57.90 mmol) in hydrogen chloride aqueous solution (12 N, 160 mL, 1.92 mol), was added a solution of NaNO₂ (15 g, 206.52 mmol) in water (20 mL) dropwise over 0.5 h period. The resulting mixture was stirred for 15 min at room temperature. After the reaction was done, the reaction mixture was extracted with ethyl acetate (500 mL×3). The combined organic phases were concentrated under reduced pressure and the residue was diluted water (300 mL). The pH value of the resulting mixture was adjusted to 8 with sodium hydroxide solution (2 M). The mixture was extracted with ethyl acetate (300 mL×3) and the aqueous layer was adjusted the pH=3 with HCl solution (3 N). The resulting mixture was extracted again with ethyl acetate (300 mL×3). The organic layers were combined and dried over anhydrous sodium sulfate and concentrated under vacuum to yield 3-(benzyloxy)-2-chloropropanoic acid as light brown oil (8.0 g, 64%). MS: 213 [M+H]⁺.

3-(benzylamino)-1,1,1-trifluoropropan-2-ol: At −10° C., to a solution of lithium trifluoromethanesulfonate (855 mg, 5.48 mmol) in acetonitrile (25 mL) was added 2-(trifluoromethyl)oxirane (6.17 g, 55.11 mmol) slowly. Then phenylmethanamine (5.57 g, 52.13 mmol) was added dropwise at −10° C. The resulting mixture was stirred for 16 h at room temperature. After the reaction was done, the reaction mixture was concentrated under reduced pressure and the residue was purified by flash chromatography eluting with ethyl acetate in hexane (0% to 10% gradient) to yield 3-(benzylamino)-1,1,1-trifluoropropan-2-ol as a white solid (7.89 g, 41%). MS: 220 [M+H]⁺.

Anti-2-N-benzyl-3-(benzyloxy)-2-chloro-N-[(2)-3,3,3-trifluoro-2-hydroxypropyl]propanamide: To a solution of 3-(benzyloxy)-2-chloropropanoic acid (6.20 g, 28.89 mmol) in dichloromethane (500 mL) was added DIEA (13.96 g, 108.05 mmol), HATU (12.35 g, 32.48 mmol), 3-(benzylamino)-1,1,1-trifluoropropan-2-ol (4.93 g, 22.49 mmol) in sequence at room temperature. The resulting solution was stirred for 16 h at room temperature. When the reaction was done, it was quenched by the addition of water (300 mL). The resulting mixture was extracted with ethyl acetate (500 mL×3). The organic phases were combined, washed with brine and dried over Na₂SO₄. The solvent was removed under reduced pressure and the residue was purified by flash chromatography eluting with ethyl acetate in hexane (0% to 10% gradient) to yield anti-2-N-benzyl-3-(benzyloxy)-2-chloro-N-[(2)-3,3,3-trifluoro-2-hydroxypropyl]propanamide as a yellow solid (1.59 g, 17%). MS: 416 [M+H]⁺.

cis-4-benzyl-2-(benzyloxymethyl)-6-(trifluoromethyl)morpholin-3-one: At −30° C., to a solution of anti-2-N-benzyl-3-(benzyloxy)-2-chloro-N-[(2)-3,3,3-trifluoro-2-hydroxypropyl]propanamide (883 mg, 2.12 mmol) in THF (150 mL) was added sodium hydride (600 mg, 25.0 mmol) in batches. The resulting mixture was stirred for 4 h at −30° C. When the reaction was done, it was quenched by the addition of ice water (200 mL). The resulting mixture was extracted with ethyl acetate (300 mL×3). The organic phases were combined, washed with brine and dried over Na₂SO₄. The solvent was removed under reduced pressure and the residue was purified by flash chromatography eluting with ethyl acetate in hexane (0% to 10% gradient) to yield cis-4-benzyl-2-[(benzyloxy)methyl]-6-(trifluoromethyl)morpholin-3-one as light red oil (639 mg, 79%). MS: 380 [M+H]⁺.

cis-4-benzyl-2-[(benzyloxy)methyl]-6-(trifluoromethyl)morpholine: To a solution of cis-4-benzyl-2-[(benzyloxy)methyl]-6-(trifluoromethyl)morpholin-3-one (639 mg, 1.68 mmol) in THF (20 mL) was added BH₃ in THF solution (1 N, 12 mL, 12 mmol) at room temperature. The resulting mixture was stirred for 3 h at room temperature. When the reaction was done, it was quenched by the addition of EtOH (40 mL). The resulting mixture was concentrated under reduced pressure and the residue was purified by flash chromatography eluting with ethyl acetate in hexane (0% to 15% gradient) to yield cis-4-benzyl-2-[(benzyloxy)methyl]-6-(trifluoromethyl)morpholine as light yellow oil (354 mg, 58%).

[cis-6-(trifluoromethyl)morpholin-2-yl]methanol: At room temperature, to a solution of cis-4-benzyl-2-[(benzyloxy)methyl]-6-(trifluoromethyl)morpholine (177 mg, 0.48 mmol) in methanol (10 mL) was added palladium carbon (87 mg, 0.82 mmol) and hydrogen chloride solution (0.5 mL, 6 mmol, 12 N) under nitrogen atmosphere. The reaction flask was vacuumed and flushed with hydrogen. The reaction mixture was hydrogenated for 12 h at room temperature under hydrogen atmosphere using a hydrogen balloon. After the reaction was done, the reaction mixture was filtered through a Celite pad and the filtrate was concentrated under reduced pressure to yield [cis-6-(trifluoromethyl)morpholin-2-yl]methanol as a light yellow solid (88 mg, 98%). MS: 186 [M+H]⁺.

### Intermediate 10: 8-[cis-2-(hydroxymethyl)-6-(trifluoromethyl)morpholin-4-yl]quinoxaline-5-carbonitrile

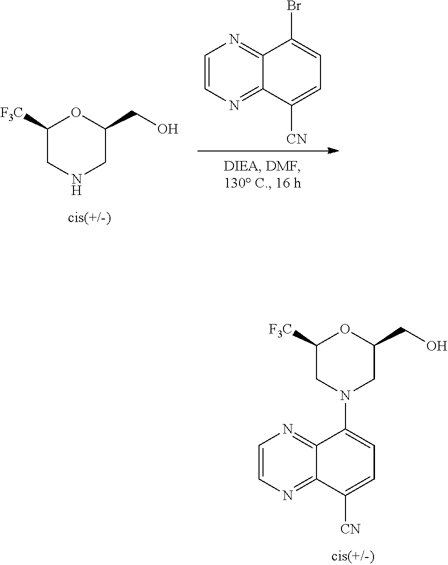 [SMILES: OC[C@H]1CNC[C@@H](C(F)(F)F)O1 ; N#Cc1ccc(Br)c2nccnc12 ; N#Cc1ccc(N2C[C@H](CO)O[C@H](C(F)(F)F)C2)c2nccnc12]

To a solution of 8-bromoquinoxaline-5-carbonitrile (221 mg, 0.96 mmol) in DMF (25 mL) was added [cis-6-(trifluoromethyl)morpholin-2-yl]methanol (260 mg, 1.36 mmol), DIEA (629 mg, 4.8 mmol) at room temperature. The resulting mixture was stirred for 16 h at 130° C. When the reaction was done, the reaction mixture was diluted with water (20 mL). The resulting mixture was extracted with ethyl acetate (50 mL×3). The organic phases were combined, washed with brine and dried over Na₂SO₄. The solvent was removed under reduced pressure and the residue was purified by flash chromatography eluting with ethyl acetate in hexane (0% to 30% gradient) to yield 8-[cis-2-(hydroxymethyl)-6-(trifluoromethyl)morpholin-4-yl]quinoxaline-5-carbonitrile as light yellow oil (100 mg, 31%). MS: 339 [M+H]⁺.

### Intermediate 11: 5-[cis-2-(hydroxymethyl)-6-(trifluoromethyl)morpholin-4-yl]quinoline-8-carbonitrile

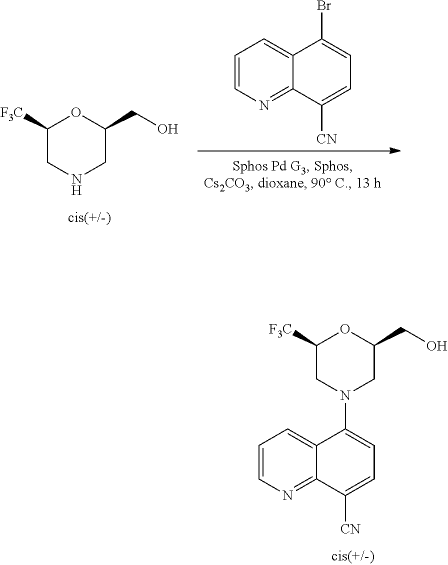 [SMILES: OC[C@H]1CNC[C@@H](C(F)(F)F)O1 ; N#Cc1ccc(Br)c2cccnc12 ; N#Cc1ccc(N2C[C@H](CO)O[C@H](C(F)(F)F)C2)c2cccnc12]

To a solution of 5-bromoquinoline-8-carbonitrile (600 mg, 2.57 mmol) in dioxane (30 mL) was added [cis-6-(trifluoromethyl)morpholin-2-yl]methanol (540 mg, 2.92 mmol), SPhos (210 mg, 0.51 mmol), SPhos Palladacycle Gen.3 (399 mg, 0.51 mmol), Cs₂CO₃ (2510 mg, 7.71 mmol) at room temperature under nitrogen atmosphere. The resulting mixture was stirred for 13 h at 90° C. under nitrogen atmosphere. After the reaction was done, the reaction mixture was diluted with water (50 mL). The resulting mixture was extracted with ethyl acetate (150 mL×3). The organic phases were combined, washed with brine and dried over Na₂SO₄. The solvent was removed under reduced pressure and the residue was purified by flash chromatography eluting with EtOAc in hexane (0% to 40% gradient) to yield 5-[cis-2-(hydroxymethyl)-6-(trifluoromethyl)morpholin-4-yl]quinoline-8-carbonitrile as a yellow solid (300 mg, 34%). MS: 338 [M+H]⁺.

### Intermediate 12: 5-[cis-2-(hydroxymethyl)-6-(trifluoromethyl)morpholin-4-yl]-1,7-naphthyridine-8-carbonitrile

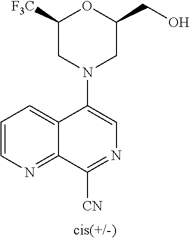 [SMILES: N#Cc1ncc(N2C[C@H](CO)O[C@H](C(F)(F)F)C2)c2cccnc12]

The title compound was prepared from 5-bromo-1,7-naphthyridine-8-carbonitrile and [cis-6-(trifluoromethyl)morpholin-2-yl]methanol using the same method for Intermediate 11 as a yellow solid (60% yield). MS: 339 [M+H]⁺.

### Intermediate 13: [(2R,6R)-4-(7-Fluoro-8-methyl-quinolin-5-yl)-6-methyl-morpholin-2-yl]-methanol

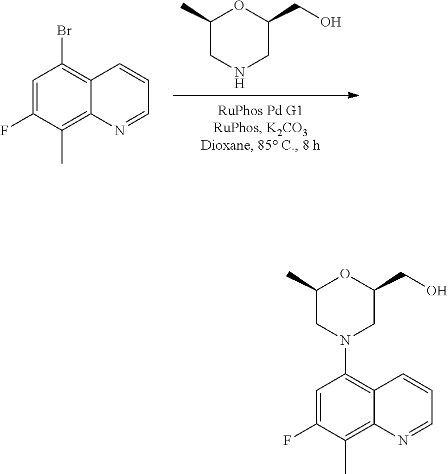

In a 5 mL microwave vial, 5-Bromo-7-fluoro-8-methyl-quinoline (200.0 mg; 0.83 mmol; 1.0 eq.), ((2R,6R)-6-Methyl-morpholin-2-yl)-methanol (109.28 mg; 0.83 mmol; 1.0 eq.), RuPhos Pd (34.84 mg; 0.04 mmol; 0.05 eq.), RuPhos (38.87 mg; 0.08 mmol; 0.10 eq.) and potassium carbonate (345.41 mg; 2.50 mmol; 3.0 eq.) were dissolved in anhydrous Dioxane (20 mL). The tube was sealed and flushed with nitrogen for 5 minutes and the suspension was microwaved at 100° C. for 8 h. The reaction mixture was filtered through celite. The filtrate was concentrated under reduced pressure and re-dissolved in DCM. The solution was absorbed on a PuriFlash celite 5 g column and purified by chromatography on a PuriFlash 12 g 30u column (Hexanes-AcOEt 10% for 5 column volumes, Hexanes-AcOEt 40-60% for 18 minutes. The pure fractions were concentrated under reduced pressure and the yellow gum was dried under vacuo to give the title (45.0 mg; 017%). MS: 291 [M+H]⁺.

### Intermediate 14: [(2R,6R)-6-methyl-4-(8-methylquinolin-5-yl)morpholin-2-yl]methanol

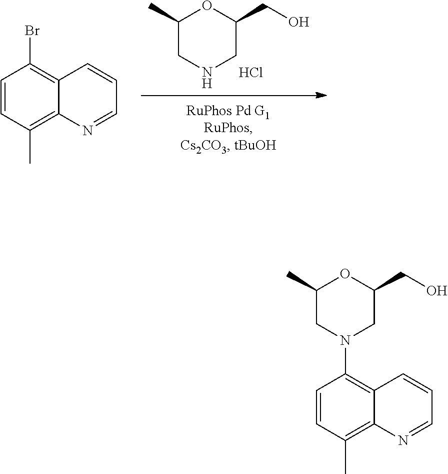

To a microwave vial was added 5-bromo-8-methylquinoline (532.0 mg; 2.40 mmol; 1.0 eq.), ((2R,6R)-6-Methyl-morpholin-2-yl)-methanol hydrochloride (401.57 mg; 2.40 mmol; 1.0 eq.), chloro-(2-dicyclohexylphosphino-2′,6′-diisopropoxy-1,1′-biphenyl)[2-(2-aminoethyl) phenyl]palladium(ii)-methyl-t-butyl ether adduct (58.7 mg; 0.07 mmol; 0.03 eq.), 2-dicyclohexylphosphino-2′,6′-diisopropoxy-1,1′-biphenyl (33.54 mg; 0.07 mmol; 0.03 eq.), cesium carbonate (1951.27 mg; 5.99 mmol; 2.50 eq.) and tBuOH (12.0 ml). The mixture was heated to 100° C. in microwave for 4.5 h., The mixture was diluted with EtOAc and filtered. The filtrate was washed with water and brine, dried, and concentrated. The residue was purified by Biotage to give the title compound as a white solid (103 mg, 15%). MS: 273 [M+H]⁺.

### Intermediate 15: 5-[(2R,6R)-2-(hydroxymethyl)-6-methylmorpholin-4-yl]-1,7-naphthyridine-8-carbonitrile

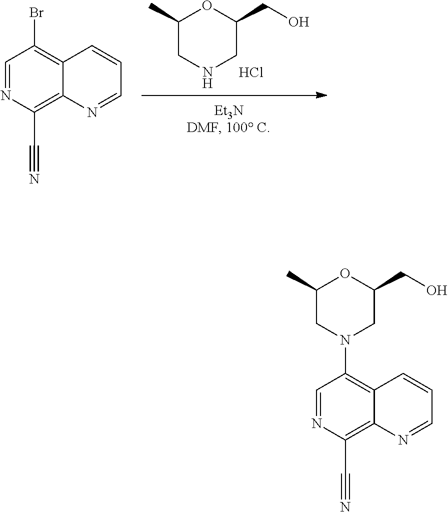

To a microwave vial with 5-bromo-[1,7]naphthyridine-8-carbonitrile (1.07 g; 4.44 mmol; 1.0 eq.) was added ((2R,6R)-6-Methyl-morpholin-2-yl)-methanol hydrochloride (0.74 g; 4.44 mmol; 1.0 eq.), Triethyl-amine (1.25 ml; 8.89 mmol; 2.0 eq.) and DMF (10 ml). The mixture was stirred in microwave at 100° C. for 2 h. The mixture was diluted with EtOAc, and filtered. The filtrate was washed with water and brine, dried, and concentrated. The residue was purified by flash chromatography (hexanes in EtOAc) to yield the title compound as a pale yellow solid (29.5 mg, 41%). MS: 285 [M+H]⁺.

### Intermediate 16: 5-((2R,6R)-2-Hydroxymethyl-6-methyl-morpholin-4-yl)-quinazoline-8-carbonitrile

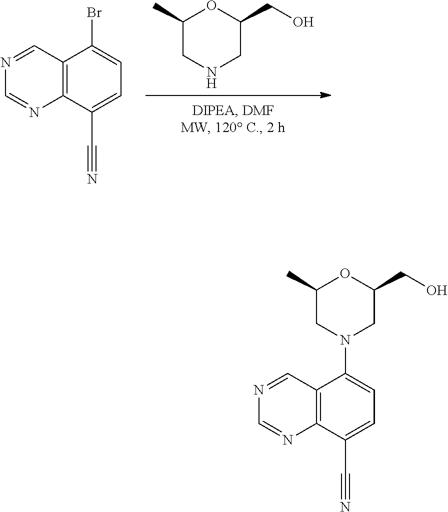

In a 25 mL microwave vial, ((2R,6R)-6-Methyl-morpholin-2-yl)-methanol (1.0 g; 5.97 mmol; 1.0 eq.), 5-Bromo-quinazoline-8-carbonitrile (1.40 g; 5.97 mmol; 1.0 eq.) and DIEA (2.96 mL; 17.90 mmol; 3.0 eq.) were dissolved in anhydrous DMF (10.0 mL). The tube was sealed and the yellow solution was microwaved at 120° C. for 5 h. The yellow solution was concentrated under reduced pressure. Water (50 mL) was added to the residue then the solid suspension filtered and dried to give 5-((2R,6R)-2-Hydroxymethyl-6-methyl-morpholin-4-yl)-quinazoline-8-carbonitrile (1280.0 mg; 75%) as a brown solid. MS: 285 [M+H]⁺.

### Intermediate 17: [(2R,6R)-6-Methyl-4-(8-trifluoromethyl-quinolin-5-yl)-morpholin-2-yl]-methanol

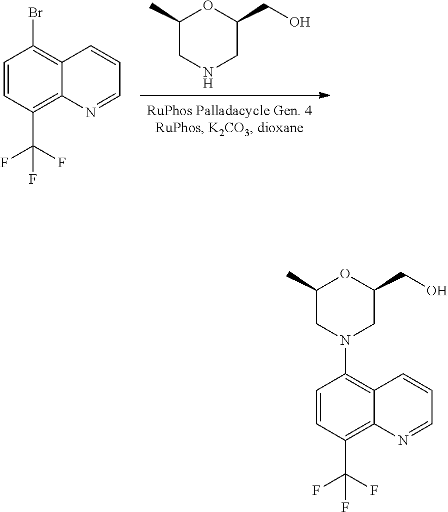

In a 25 mL microwave vial, 5-Bromo-8-trifluoromethyl-quinoline (500.0 mg; 1.81 mmol; 1.0 eq.), ((2R,6R)-6-Methyl-morpholin-2-yl)-methanol (285.10 mg; 2.17 mmol; 1.20 eq.), methanesulfonato(2-dicyclohexylphosphino-2′,6′-di-i-propoxy-1,1′-biphenyl)(2′-amino-1,1′-biphenyl-2-yl)palladium(ii) (75.74 mg; 0.09 mmol; 0.05 eq.), 2-dicyclohexylphosphino-2′,6′-di-i-propoxy-1,1′-biphenyl (84.52 mg; 0.18 mmol; 0.10 eq.) and potassium carbonate (750.98 mg; 5.43 mmol; 3.0 eq.) were dissolved in anhydrous Dioxane (10.0 ml). The tube was sealed and flushed with nitrogen for 5 minutes and the suspension was microwaved at 100° C. for 8 h. The reaction mixture was filtered through celite. The filtrate was concentrated under reduced pressure and re-dissolved in DCM. The solution was absorbed on a PuriFlash celite 5 g column and purified by chromatography on a PuriFlash 10 g 30u column (Hexanes-AcOEt 10% for 5 column volumes, Hexanes-AcOEt 40-60% for 18 minutes. The pure fractions were concentrated under reduced pressure and the light yellow oil was dried under vacuo to give [(2R,6R)-6-Methyl-4-(8-trifluoromethyl-quinolin-5-yl)-morpholin-2-yl]-methanol (245.0 mg; 41%). MS:327 [M+H]⁺.

### Intermediate 18: (2R,6R)-4-(7-Fluoro-8-methyl-quinolin-5-yl)-6-methyl-morpholine-2-carboxylic acid

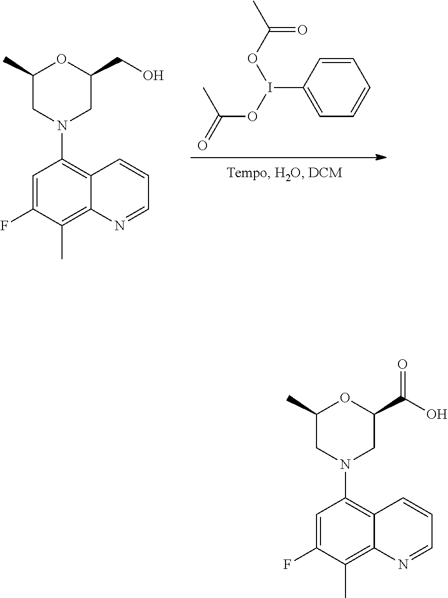 [SMILES: Cc1c(F)cc(N2C[C@H](CO)O[C@H](C)C2)c2cccnc12 ; CC(=O)OI(OC(C)=O)c1ccccc1 ; Cc1c(F)cc(N2C[C@@H](C)O[C@@H](C(=O)O)C2)c2cccnc12]

Into a 50 mL round-bottom flask, was placed [(2R,6R)-4-(7-Fluoro-8-methyl-quinolin-5-yl)-6-methyl-morpholin-2-yl]-methanol (140.0 mg; 0.48 mmol; 1.0 eq.) and DCM (15.0 mL). The resulting solution was stirred for 5 minutes at 0° C. in a water/ice bath, then (diacetoxyiodo)benzene (0.31 g; 0.96 mmol; 2.0 eq.) was added. After raising the temperature to 10° C., tempo (15.07 mg; 0.10 mmol; 0.20 eq.) and water (0.60 ml) were added. The resulting solution was stirred for an additional 20 minutes while the temperature was maintained at 10° C. in a water/ice bath. The reaction solution was stirring for an additional 2 h at 25° C. after which the yellow solid suspension become a brown solution. LC/MS showed the reaction was complete. The reaction was then quenched by the addition of 0.5 mL of 10% sodium thiosulfate(aq), and was stirred for another 45 minutes. The resulting mixture was concentrated under vacuum. The residue was dispersed in a mixture of 1:1 DCM/methanol, filtered and the filtrate evaporated to give (2R,6R)-4-(7-Fluoro-8-methyl-quinolin-5-yl)-6-methyl-morpholine-2-carboxylic acid (106.0 mg; crude) as a yellow solid. MS: 305 [M+H]⁺.

### Intermediate 19: cis-4-(8-cyano-1,7-naphthyridin-5-yl)-6-(trifluoromethyl)morpholine-2-carboxylic acid

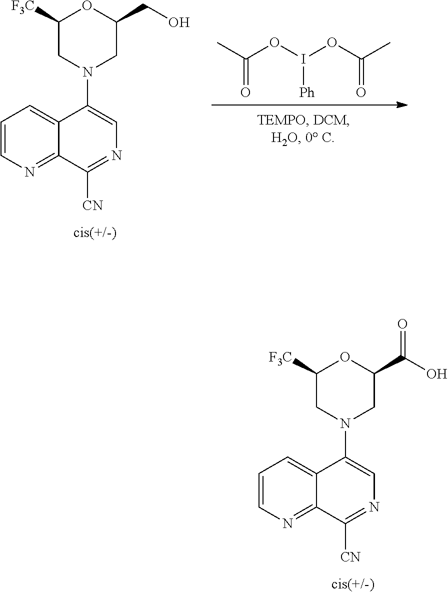 [SMILES: N#Cc1ncc(N2C[C@H](CO)O[C@H](C(F)(F)F)C2)c2cccnc12 ; CC(=O)OI(OC(C)=O)c1ccccc1 ; N#Cc1ncc(N2C[C@@H](C(F)(F)F)O[C@@H](C(=O)O)C2)c2cccnc12]

At 0° C., to a mixture of 5-[cis-2-(hydroxymethyl)-6-(trifluoromethyl)morpholin-4-yl]-1,7-naphthyridine-8-carbonitrile (313 mg, 0.93 mmol) in dichloromethane (38 mL) and water (19 mL) was added (diacetoxyiodo)benzene (686 mg, 2.13 mmol) and TEMPO (36 mg, 0.23 mmol) at 0° C. The resulting mixture was stirred for 8 h at 0° C. When the reaction was done, it was quenched by the addition of MeOH (10 mL). The reaction mixture was concentrated under reduced pressure and then azeotroped with toluene to remove most of the solvent. The residue was purified by flash chromatography eluting with MeOH in DCM (0% to 15% gradient) to yield cis-4-(8-cyano-1,7-naphthyridin-5-yl)-6-(trifluoromethyl)morpholine-2-carboxylic acid as brown oil (134 mg, 78%). MS: 353 [M+H]⁺.

### Intermediate 20: cis-4-(8-cyanoquinolin-5-yl)-6-(trifluoromethyl)morpholine-2-carboxylic acid

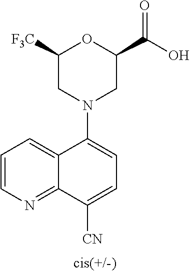 [SMILES: N#Cc1ccc(N2C[C@@H](C(F)(F)F)O[C@@H](C(=O)O)C2)c2cccnc12]

The title compound was prepared from 5-[cis-2-(hydroxymethyl)-6-(trifluoromethyl)morpholin-4-yl]quinoline-8-carbonitrile using the same method for Intermediate 19 as yellow oil (48% yield). MS: 352 [M+H]⁺.

### Intermediate 21: trans-5-methylpiperidin-3-yl 4-nitrobenzoate

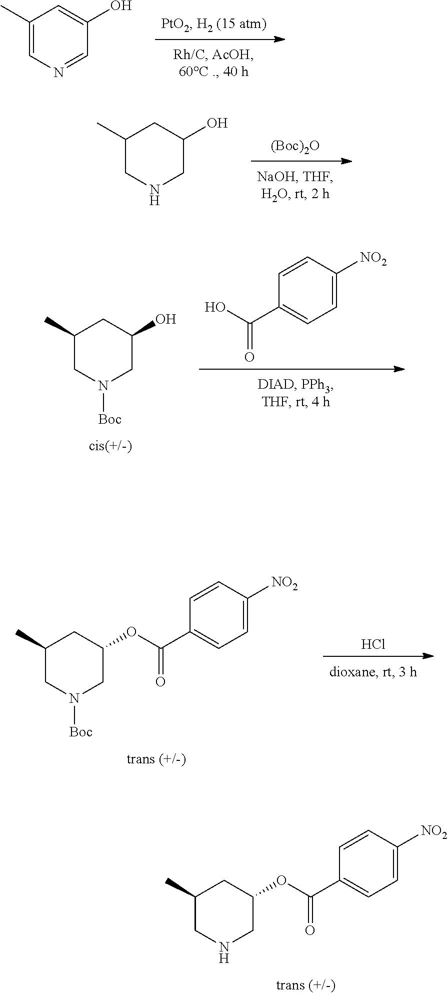

5-Methylpiperidin-3-ol: At room temperature, to a solution of 5-methylpyridin-3-ol (4.90 g, 44.90 mmol) in acetic acid (200 mL) was added Rh/C (1.42 g, 13.85 mmol), PtO₂ (1.42 g, 6.28 mmol) under nitrogen atmosphere. The reaction tank was vacuumed and flushed with hydrogen. The reaction mixture was hydrogenated for 12 h at room temperature under hydrogen atmosphere (15 atm). After the reaction was done, the reaction mixture was filtered through a Celite pad and the filtrate was concentrated under reduced pressure to yield 5-methylpiperidin-3-ol as brown oil (4.50 g, cis/trans=4:1, 87%). MS: 116.2 [M+H]⁺.

cis-tert-Butyl 3-hydroxy-5-methylpiperidine-1-carboxylate: At 0° C., to a solution 5-methylpiperidin-3-ol (4.0 g, 34.73 mmol) in tetrahydrofuran (100 mL) was added sodium hydroxide aqueous solution (2 N, 30 mL, 60.0 mmol). To the above stirred solution was added a solution of (Boc)₂O (10.29 g, 47.15 mmol) in tetrahydrofuran (50 mL) dropwise over 15 min period at room temperature. The reaction mixture was stirred for 2 h at room temperature. When the reaction was done, the reaction mixture was diluted with water (300 mL) and extracted with ethyl acetate (300 mL×3). The organic phases were combined, washed with brine and dried over Na₂SO₄. The solvent was removed under reduced pressure and the residue was purified by flash chromatography eluting with EtOAc in hexane (0% to 40% gradient) to yield cis-tert-butyl 3-hydroxy-5-methylpiperidine-1-carboxylate as a yellow solid (4.50 g, 60%). MS: 160.3 [M+H]⁺.

trans-tert-Butyl 3-methyl-5-[(4-nitrophenyl)carbonyloxy]piperidine-1-carboxylate: To a solution of cis-tert-butyl 3-hydroxy-5-methylpiperidine-1-carboxylate (2.70 g, 12.54 mmol) in tetrahydrofuran (60 mL) was added 4-nitrobenzoic acid (3.52 g, 21.06 mmol), PPh₃ (5.85 g, 22.31 mmol), DIAD (4.48 g, 22.18 mmol) at room temperature. The resulting mixture was stirred for 4 h at room temperature. When the reaction was done, it was quenched by the addition of sat. NH₄C₁ solution (200 mL). The resulting mixture was extracted with ethyl acetate (300 mL×3). The organic phases were combined, washed with brine and dried over Na₂SO₄. The solvent was removed under reduced pressure and the residue was purified by flash chromatography eluting with EtOAc in hexane (0% to 50% gradient) to yield trans-tert-butyl 3-methyl-5-[(4-nitrophenyl)carbonyloxy]piperidine-1-carboxylate as a yellow solid (4.0 g, 92%). MS: 308.9 [M+H]⁺.

trans-5-methylpiperidin-3-yl 4-nitrobenzoate: To a solution of trans-tert-butyl 3-methyl-5-[(4-nitrophenyl)carbonyloxy]piperidine-1-carboxylate (4.0 g, 10.97 mmol) in dioxane (150 mL) was added hydrogen chloride aqueous solution (6 N, 15 mL, 90.0 mmol) at room temperature. The resulting mixture was stirred for 3 h at room temperature. When the reaction was done, the pH value of the mixture was adjusted to 10 with sat. sodium carbonate solution, and the resulting mixture was concentrated under vacuum to remove organic solvent. The remaining mixture was extracted with ethyl acetate (100 mL×3). The organic phases were combined, washed with brine and dried over Na₂SO₄. The solvent was removed under reduced pressure to yield trans-5-methylpiperidin-3-yl 4-nitrobenzoate as a yellow solid (3.70 g, crude). MS: 265.0 [M+H]⁺.

### Intermediate 22: trans-1-(8-cyanoquinoxalin-5-yl)-5-methylpiperidin-3-yl 4-nitrobenzoate

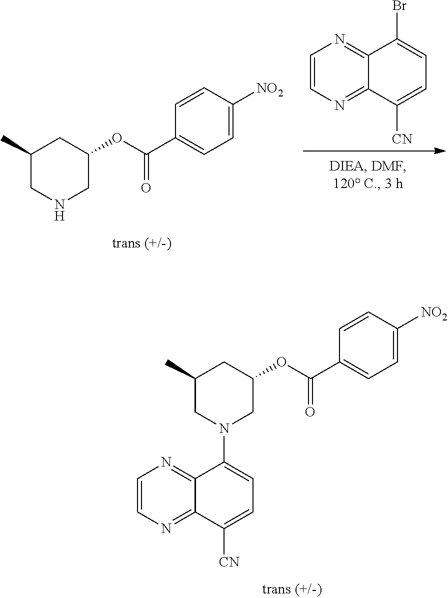 [SMILES: C[C@@H]1CNC[C@@H](OC(=O)c2ccc([N+](=O)[O-])cc2)C1 ; N#Cc1ccc(Br)c2nccnc12 ; C[C@H]1C[C@H](OC(=O)c2ccc([N+](=O)[O-])cc2)CN(c2ccc(C#N)c3nccnc23)C1]

To a solution of trans-5-methylpiperidin-3-yl 4-nitrobenzoate (3.70 g, crude) in N,N-dimethylformamide (100 mL) was added 8-bromoquinoxaline-5-carbonitrile (3.08 g, 13.15 mmol) and DIEA (5.14 g, 39.77 mmol) at room temperature. The resulting mixture was stirred for 3 h at 120° C. When the reaction was done, the reaction mixture was concentrated under reduced pressure and the residue was purified by flash chromatography eluting with EtOAc in hexane (0% to 10% gradient) to yield trans-1-(8-cyanoquinoxalin-5-yl)-5-methylpiperidin-3-yl 4-nitrobenzoate as a yellow solid (2.62 g, 57% for 2 steps). MS: 418.0 [M+H]⁺.

### Intermediate 23: trans-5-methyl-1-[8-(trifluoromethyl)quinoxalin-5-yl]piperidin-3-yl 4-nitrobenzoate

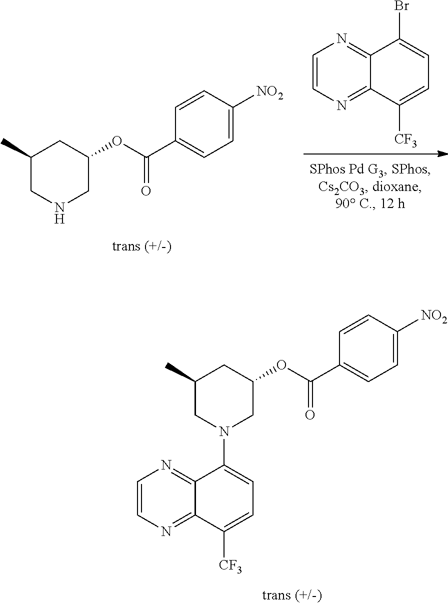 [SMILES: C[C@@H]1CNC[C@@H](OC(=O)c2ccc([N+](=O)[O-])cc2)C1 ; FC(F)(F)c1ccc(Br)c2nccnc12 ; C[C@H]1C[C@H](OC(=O)c2ccc([N+](=O)[O-])cc2)CN(c2ccc(C(F)(F)F)c3nccnc23)C1]

In a 50 mL sealed tube, to a solution of 5-bromo-8-(trifluoromethyl)quinoxaline (450 mg, 1.62 mmol) in dioxane (15 mL) was added trans-5-methylpiperidin-3-yl 4-nitrobenzoate (867 mg, 3.25 mmol), 3rd Generation SPhos precatalyst (253 mg, 0.32 mmol), SPhos (373 mg, 0.91 mmol), Cs₂CO₃ (1085 mg, 3.33 mmol) at room temperature under nitrogen atmosphere. The resulting mixture was stirred for 12 h at 90° C. under nitrogen atmosphere. When the reaction was done, the reaction mixture was concentrated under reduced pressure and the residue was purified by flash chromatography eluting with MeOH in DCM (0% to 10% gradient) to yield trans-5-methyl-1-[8-(trifluoromethyl)quinoxalin-5-yl]piperidin-3-yl 4-nitrobenzoate as a yellow solid (144 mg, 19%). MS: 461.0 [M+H]⁺.

### Preparation of Examples

Examples were prepared according to the methods describes below using the intermediates above or the intermediates in WO 2017/106607A1 and commercially available reagents.

### Example 1: 8-[(3S,5R)-3-methyl-5-[2-(4-methylpiperazin-1-yl)ethoxy]piperidin-1-yl]quinoxaline-5-carbonitrile

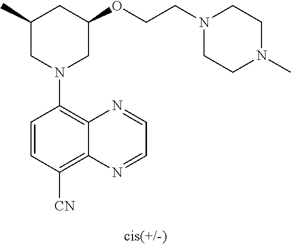

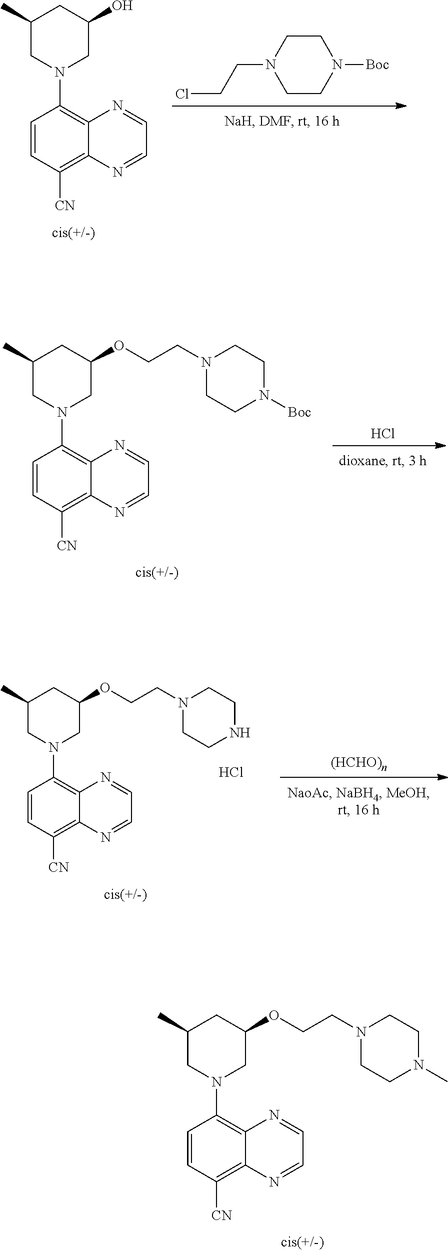

tert-butyl 4-(2-[[cis-1-(8-cyanoquinoxalin-5-yl)-5-methylpiperidin-3-yl]oxy]ethyl) piperazine-1-carboxylate: To a solution of 8-[cis-3-hydroxy-5-methylpiperidin-1-yl]quinoxaline-5-carbonitrile (300 mg, 1.01 mmol, 1.0 equiv) in DMF (20.0 mL) was added sodium hydride (804 mg, 33.50 mmol) at room temperature. The resulting mixture was stirred for 20 min at room temperature, and then was added tert-butyl 4-(2-chloroethyl)piperazine-1-carboxylate (788 mg, 3.17 mmol). The reaction mixture was stirred for 16 h at room temperature. When the reaction was done, it was quenched by the addition of water (10 mL). The resulting mixture was extracted with ethyl acetate (30 mL×3). The organic phases were combined, washed with brine and dried over Na₂SO₄. The solvent was removed under reduced pressure and the residue was purified by flash chromatography eluting with MeOH in DCM (0% to 40% gradient) to yield the title compound as a yellow solid (170 mg, 35%). MS: 481 [M+H]⁺.

8-[cis-3-methyl-5-[2-(piperazin-1-yl)ethoxy]piperidin-1-yl]quinoxaline-5-carbonitrile hydrochloride: To a solution of tert-butyl 4-(2-[[cis-1-(8-cyanoquinoxalin-5-yl)-5-methylpiperidin-3-yl]oxy]ethyl)piperazine-1-carboxylate (145 mg, 0.30 mmol) in dioxane (50.0 mL) was added hydrogen chloride solution (12 N, 1 mL, 12 mmol) at room temperature. The resulting mixture was stirred for 3 h at room temperature and then was concentrated under reduced pressure to yield the title compound as a yellow solid (85 mg, 74%). MS: 381 [M+H]⁺.

8-[cis-3-methyl-5-[2-(4-methylpiperazin-1-yl)ethoxy]piperidin-1-yl]quinoxaline-5-carbonitrile: At room temperature, to a solution of 8-[cis-3-methyl-5-[2-(piperazin-1-yl)ethoxy]piperidin-1-yl]quinoxaline-5-carbonitrile hydrochloride (53 mg, 0.13 mmol) in methanol (10 mL) was added NaOAc (308 mg, 3.75 mmol), (HCHO)ₙ (108 mg, 1.20 mmol), NaBH₄ (33 mg, 0.87 mmol) in sequence. The resulting mixture was stirred for 16 h at room temperature. When the reaction was done, it was quenched by the addition of water (10 mL). The resulting mixture was extracted with ethyl acetate (30 mL×3). The organic phases were combined, washed with brine and dried over Na₂SO₄. The solvent was removed under reduced pressure and the residue was purified by prep-HPLC under the following conditions: Column, XBridge Shield RP18 OBD Column, 19×150 mm 5 um; acetonitrile in water (with 10 mmol/L NH₄HCO₃ and 0.1% NH₃.H₂O), 35% to 65% gradient in 10 min; Detector, UV 254 nm. The title compound was obtained as a light yellow solid (11 mg, 21%). MS: 395 [M+H]⁺. H NMR (400 MHz, Methanol-d₄, ppm) δ 8.95 (d, J=1.8 Hz, 1H), 8.90 (d, J=1.7 Hz, 1H), 8.10 (d, J=8.4 Hz, 1H), 7.24 (d, J=8.4 Hz, 1H), 4.64-4.56 (m, 1H), 4.12-4.04 (m, 1H), 3.85-3.70 (m, 3H), 2.81-2.46 (m, 12H), 2.35-2.31 (m, 1H), 2.29 (s, 3H), 2.06-2.01 (m, 1H), 1.16-1.04 (m, 1H), 1.05 (d, J=6.6 Hz, 3H).

The following compounds were synthesized in an analogous manner.

### Example 2: 5-[cis-3-methyl-5-[2-(4-methylpiperazin-1-yl)ethoxy]piperidin-1-yl]-8-(trifluoromethyl)quinoline

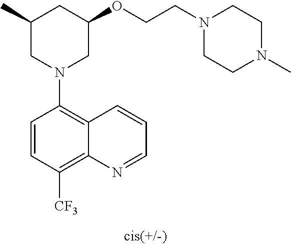

The title compound was prepared from cis-5-methyl-1-[8-(trifluoromethyl)quinolin-5-yl]piperidin-3-ol and tert-butyl 4-(2-chloroethyl)piperazine-1-carboxylate. MS: 437 [M+H]⁺. H NMR (400 MHz, Methanol-d₄, ppm) δ 8.95 (dd, J=4.2, 1.7 Hz, 1H), 8.60 (dd, J=8.6, 1.8 Hz, 1H), 8.05 (d, J=8.0 Hz, 1H), 7.62 (dd, J=8.6, 4.2 Hz, 1H), 7.25 (d, J=8.0 Hz, 1H), 3.87-3.62 (m, 4H), 3.39-3.33 (m, 1H), 2.84-2.22 (m, 16H), 2.17-2.06 (m, 1H), 1.16-1.06 (m, 1H), 1.04 (d, J=6.6 Hz, 3H).

### Example 3: 5-[cis-3-methyl-5-[2-(piperidin-1-yl)ethoxy]piperidin-1-yl]-8-(trifluoromethyl)quinolone

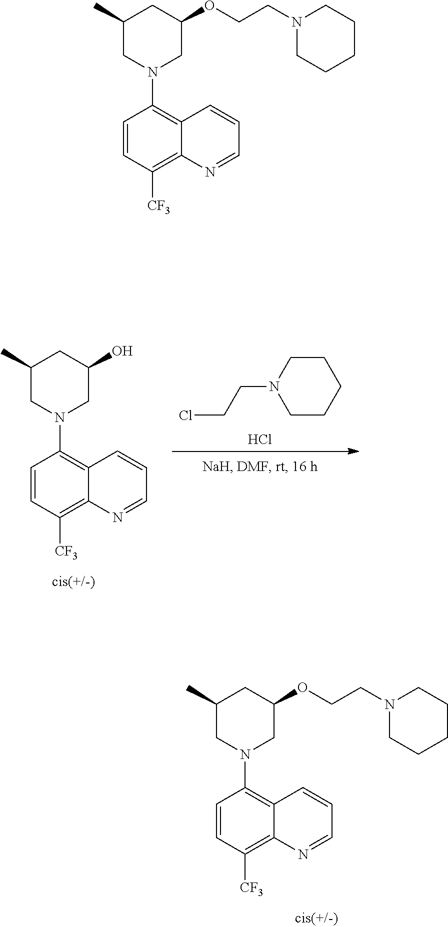 [SMILES: C[C@H]1C[C@@H](OCCN2CCCCC2)CN(c2ccc(C(F)(F)F)c3ncccc23)C1 ; C[C@H]1C[C@@H](O)CN(c2ccc(C(F)(F)F)c3ncccc23)C1 ; ClCCN1CCCCC1 ; Cl ; C[C@H]1C[C@@H](OCCN2CCCCC2)CN(c2ccc(C(F)(F)F)c3ncccc23)C1]

To a solution of cis-5-methyl-1-[8-(trifluoromethyl)quinolin-5-yl]piperidin-3-ol (85 mg, 0.27 mmol) in DMF (5 mL) was added sodium hydride (232 mg, 9.68 mmol) at room temperature. The resulting mixture was stirred for 10 min at room temperature and then was added 1-(2-chloroethyl)piperidine hydrochloride (113 mg, 0.61 mmol) at room temperature. The reaction mixture was stirred for 16 h at room temperature. When the reaction was done, it was quenched by the addition of water (10 mL). The resulting mixture was extracted with DCM (30 mL×3). The organic phases were combined, washed with brine and dried over Na₂SO₄. The solvent was removed under reduced pressure and the residue was purified by prep-HPLC under the following conditions: Column, XBridge Shield RP18 OBD, 150×190 mm, 5 um; acetonitrile in water (with 10 mmol/L NH₄HCO₃ and 0.1% NH₃.H₂O), 45% to 75% gradient in 8 min; Detector, UV 254 nm. 5-[cis-3-methyl-5-[2-(piperidin-1-yl)ethoxy]piperidin-1-yl]-8-(trifluoromethyl)quinolone was obtained as yellow solid (28 mg, 24%). MS: 422 [M+H]⁺. ¹H NMR (400 MHz, Methanol-d₄, ppm) δ 8.95 (dd, J=4.2, 1.8 Hz, 1H), 8.60 (dd, J=8.6, 1.8 Hz, 1H), 8.05 (d, J=8.1 Hz, 1H), 7.62 (dd, J=8.6, 4.2 Hz, 1H), 7.25 (d, J=8.0 Hz, 1H), 3.89-3.59 (m, 4H), 3.39-3.33 (m, 1H), 2.64-2.39 (m, 8H), 2.38-2.28 (m, 1H), 2.16-2.06 (m, 1H), 1.67-1.57 (m, 4H), 1.53-1.46 (m, 2H), 1.16-1.06 (m, 1H), 1.04 (d, J=6.6 Hz, 3H).

The following compound was synthesized in an analogous manner.

### Example 4: diethyl(2-[[cis-5-methyl-1-[8-(trifluoromethyl)quinolin-5-yl]piperidin-3-yl]oxy]ethyl)amine

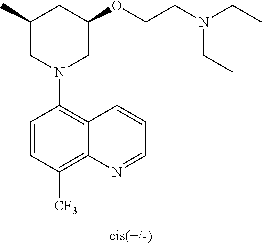 [SMILES: CCN(CC)CCO[C@@H]1C[C@H](C)CN(c2ccc(C(F)(F)F)c3ncccc23)C1]

The title compound was prepared from (3R,5S)-5-methyl-1-[8-(trifluoromethyl)quinolin-5-yl]piperidin-3-ol and (2-chloroethyl)diethylamine hydrochloride. MS: 410 [M+H]⁺. ¹H NMR (300 MHz, Methanol-d₄, ppm) δ 8.90 (dd, J=4.2, 1.8 Hz, 1H), 8.55 (dd, J=8.6, 1.8 Hz, 1H), 8.0 (d, J=8.0 Hz, 1H), 7.57 (dd, J=8.6, 4.2 Hz, 1H), 7.20 (d, J=8.0 Hz, 1H), 3.85-3.56 (m, 4H), 3.36-3.28 (m, 1H), 2.72-2.48 (m, 7H), 2.45-2.24 (m, 2H), 2.11-2.05 (m, 1H), 1.08-0.95 (m, 10H).

### Example 5: 8-[(3S,5R)-3-methyl-5-[2-(piperidin-1-yl)ethoxy]piperidin-1-yl]quinoxaline-5-carbonitrile

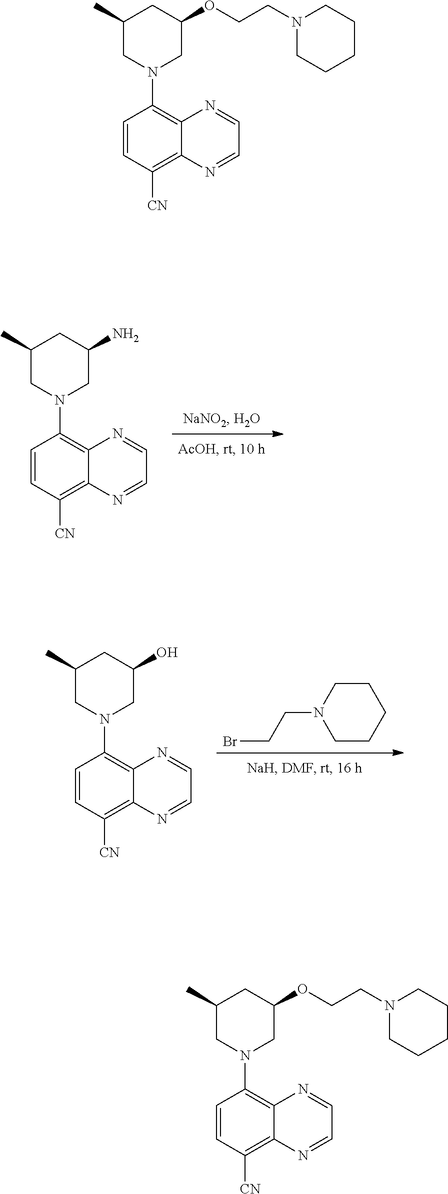 [SMILES: C[C@H]1C[C@@H](OCCN2CCCCC2)CN(c2ccc(C#N)c3nccnc23)C1 ; C[C@H]1C[C@@H](N)CN(c2ccc(C#N)c3nccnc23)C1 ; C[C@H]1C[C@@H](O)CN(c2ccc(C#N)c3nccnc23)C1 ; BrCCN1CCCCC1 ; C[C@H]1C[C@@H](OCCN2CCCCC2)CN(c2ccc(C#N)c3nccnc23)C1]

8-[(3R,5S)-3-hydroxy-5-methylpiperidin-1-yl]quinoxaline-5-carbonitrile: At 0° C., to a solution of 8-[(3R,5S)-3-amino-5-methylpiperidin-1-yl]quinoxaline-5-carbonitrile (178 mg, 0.67 mmol) in AcOH (5 mL) was added a solution of NaNO₂ (229 mg, 3.33 mmol) in water (1 mL) dropwise. The resulting solution was stirred for 10 h at room temperature. After the reaction was done, the reaction mixture was concentrated under reduced pressure and the residue was purified by prep-HPLC under the following conditions: column, XBridge BEH130 Prep C18 OBD column, 19×150 mm, 5 um, 13 nm; mobile phase, MeOH in water (with 10 mmol/L NH₄HCO₃), 30% to 80% gradient in 10 min; detector, UV 254 nm. 8-[(3R,5S)-3-hydroxy-5-methylpiperidin-1-yl]quinoxaline-5-carbonitrile was obtained as yellow solid (30 mg, 17%). MS: 269 [M+H]⁺.

8-[(3S,5R)-3-methyl-5-[2-(piperidin-1-yl)ethoxy]piperidin-1-yl]quinoxaline-5-carbonitrile: At 0° C., to a solution of 8-[(3R,5S)-3-hydroxy-5-methylpiperidin-1-yl]quinoxaline-5-carbonitrile (27 mg, 0.10 mmol) in DMF (5 mL) was added sodium hydride (5 mg, 0.20 mmol). The resulting mixture was stirred for 15 min at 0° C., and then was added 1-(2-chloroethyl)piperidine (38 mg, 0.21 mmol). The reaction mixture was stirred for 16 h at room temperature. When the reaction was done, it was quenched by the addition of water (20 mL). The resulting mixture was extracted with DCM (30 mL×3). The organic phases were combined, washed with brine and dried over Na₂SO₄. The solvent was removed under reduced pressure and the residue was purified by prep-HPLC under the following conditions: Column, XBridge Shield RP18 OBD Column, 19×150 mm 5 um; acetonitrile in water (with 10 mmol/L NH₄HCO₃ and 0.1% NH₃.H₂O), 40% to 70% gradient in 10 min; Detector, UV 254 nm. 8-[(3S,5R)-3-methyl-5-[2-(piperidin-1-yl)ethoxy]piperidin-1-yl]quinoxaline-5-carbonitrile was obtained as a light yellow solid (14 mg, 36%). MS: 380 [M+H]⁺. ¹H NMR (400 MHz, Methanol-d₄, ppm) δ 8.94 (d, J=1.8 Hz, 1H), 8.90 (d, J=1.8 Hz, 1H), 8.10 (d, J=8.4 Hz, 1H), 7.23 (d, J=8.4 Hz, 1H), 4.65-4.55 (m, 1H), 4.12-4.03 (m, 1H), 3.85-3.71 (m, 3H), 2.79-2.45 (m, 8H), 2.35-2.27 (m, 1H), 2.05-2.01 (m, 1H), 1.68-1.55 (m, 4H), 1.54-1.45 (m, 2H), 1.21-1.09 (m, 1H), 1.05 (d, J=6.7 Hz, 3H).

The following compound was synthesized in an analogous manner.

### Example 6: 8-[(3R,5S)-3-[2-(diethylamino)ethoxy]-5-methylpiperidin-1-yl]quinoxaline-5-carbonitrile

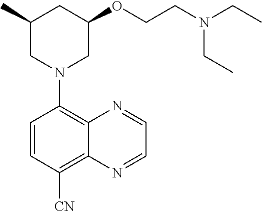 [SMILES: CCN(CC)CCO[C@@H]1C[C@H](C)CN(c2ccc(C#N)c3nccnc23)C1]

The title compound was prepared from 8-[(3R,5S)-3-hydroxy-5-methylpiperidin-1-yl]quinoxaline-5-carbonitrile and (2-chloroethyl)diethylamine hydrochloride. MS: 368 [M+H]⁺. ¹H NMR (400 MHz, Methanol-d₄, ppm) δ 8.94 (d, J=1.8 Hz, 1H), 8.90 (d, J=1.8 Hz, 1H), 8.10 (d, J=8.4 Hz, 1H), 7.23 (d, J=8.4 Hz, 1H), 4.66-4.56 (m, 1H), 4.12-4.03 (m, 1H), 3.82-3.68 (m, 3H), 2.77-2.57 (m, 8H), 2.35-2.27 (m, 1H), 2.06-2.0 (m, 1H), 1.21-1.02 (m, 10H).

### Example 7: (3R,5S)-5-Methyl-1-(8-trifluoromethyl-[1,7]naphthyridin-5-yl)-piperidin-3-ylamine hydrochloride

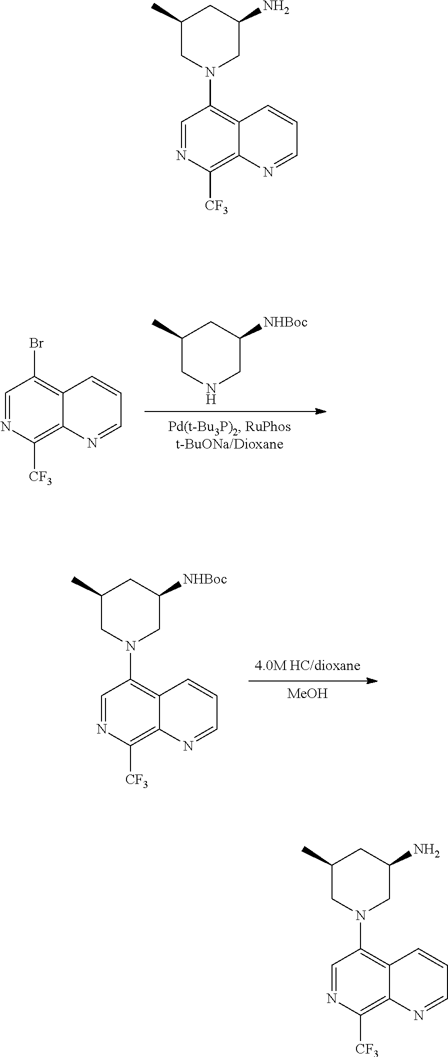

[(3R,5S)-5-Methyl-1-(8-trifluoromethyl-[1,7]naphthyridin-5-yl)-piperidin-3-yl]-carbamic acid tert-butyl ester: A solution of 5-Bromo-8-trifluoromethyl-[1,7]naphthyridine (800 mg; 2.89 mmol; 1.0 eq.), ((3R,5S)-5-Methyl-piperidin-3-yl)-carbamic acid tert-butyl ester (680 mg; 3.18 mmol; 1.10 eq.) and RuPhos (67.37 mg; 0.14 mmol; 0.05 eq.) in dioxane (10 ml) was degased, and then added 2-Methyl-propan-2-ol sodium (305 mg; 3.18 mmol; 1.10 eq.) and bis(tri-tert-butylphosphine)palladium(0) (74 mg; 0.14 mmol; 0.05 eq.) The resulted mixture was stirred at 100° C. for 2 hr. After the reaction was complete, the crude was purified by silica column, eluting with 0-55% EA/hexane, to yield the title compound (700 mg, yield 59%). LC-MS (M+1)=411.

(3R,5S)-5-Methyl-1-(8-trifluoromethyl-[1,7]naphthyridin-5-yl)-piperidin-3-ylamine hydrochloride: To a solution of [(3R,5S)-5-Methyl-1-(8-trifluoromethyl-[1,7]naphthyridin-5-yl)-piperidin-3-yl]-carbamic acid tert-butyl ester (20 mg; 0.05 mmol; 1.0 eq.) in 1 ml of methanol was added hydrogen chloride (4.0M in dioxane)(0.60 ml; 2.40 mmol; 49.25 eq.). The resulting mixture was stirred at RT for 1 hr until the reaction was completed. The reaction mixture was concentrated. The residue was suspended in ether and then filtered to yield the title compound as yellow solid (16 mg, 94%). LC-MS (M+1)=311. ¹H NMR (400 MHz, Methanol-d₄) δ 9.12 (d, J=3.9 Hz, 1H), 8.60 (d, J=8.6 Hz, 1H), 8.33 (s, 1H), 7.86 (dd, J=8.8, 4.1 Hz, 1H), 3.73 (dd, J=39.3, 11.4 Hz, 2H), 3.53 (d, J=12.0 Hz, 1H), 2.94 (t, J=10.8 Hz, 1H), 2.62 (t, J=11.5 Hz, 1H), 2.40-2.11 (m, 2H), 1.30 (q, J=12.0 Hz, 1H), 1.10 (d, J=6.4 Hz, 3H).

### Example 8: 5-((3R,5S)-3-Amino-5-methyl-piperidin-1-yl)-[1,7]naphthyridine-8-carbonitrile hydrochloride

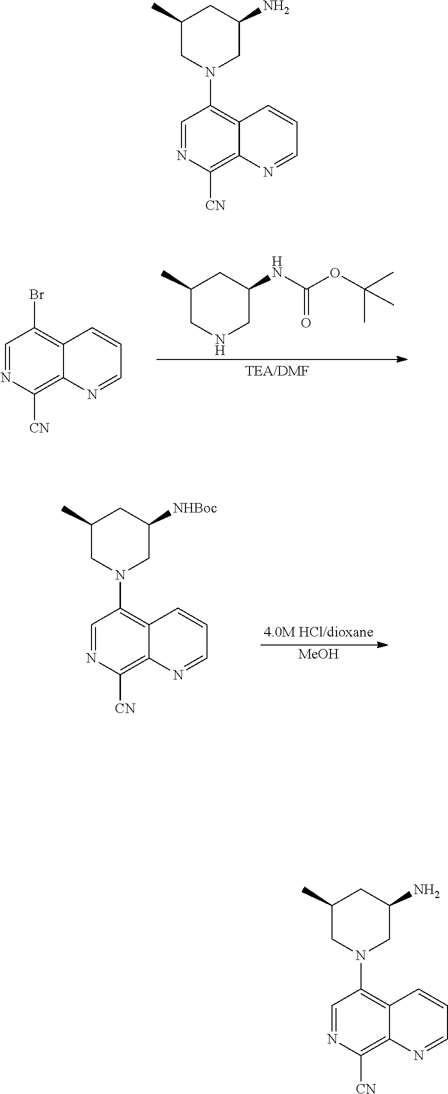

[(3R,5S)-1-(8-Cyano-[1,7]naphthyridin-5-yl)-5-methyl-piperidin-3-yl]-carbamic acid tert-butyl ester: Within a 30 mL microwave vial was added 5-Bromo-[1,7]naphthyridine-8-carbonitrile (470 mg; 2.01 mmol; 1.0 eq.), ((3R,5S)-5-Methyl-piperidin-3-yl)-carbamic acid tert-butyl ester (451 mg; 2.11 mmol; 1.05 eq.), Triethyl-amine (0.56 ml; 4.02 mmol; 2.0 eq.) and DMF (4.7 ml). The tube was sealed and microwaved at 130° C. for 3 h, until the reaction was completed. The solvent was removed and the residue was purified by silica column eluting with 0-55% EA/hexane, providing the title compound (610 mg, 82.7%) LC-MS (M+1)=368.

5-((3R,5S)-3-Amino-5-methyl-piperidin-1-yl)-[1,7]naphthyridine-8-carbonitrile hydrochloride (2): To a solution of [(3R,5S)-1-(8-Cyano-[1,7]naphthyridin-5-yl)-5-methyl-piperidin-3-yl]-carbamic acid tert-butyl ester (20 mg; 0.05 mmol; 1.0 eq.) in 1 ml methanol was added hydrogen chloride (4.0M in dioxane) (0.27 ml; 1.09 mmol; 20.0 eq.), and the reaction was stirred at RT for 3 hr until the reaction was completed. The solvent was removed to provide a yellow product as the title compound, quantitively yield. LC-MS (M+1)=268. ¹H NMR (400 MHz, Methanol-d₄) δ 9.15 (dd, J=4.1, 1.2 Hz, 1H), 8.66-8.56 (m, 1H), 8.41 (s, 1H), 7.87 (dd, J=8.6, 4.1 Hz, 1H), 3.87 (dd, J=11.1, 3.1 Hz, 1H), 3.80-3.72 (m, 1H), 3.67 (s, 1H), 3.64-3.54 (m, 2H), 3.37 (s, 1H), 2.99 (t, J=11.0 Hz, 1H), 2.68 (t, J=11.6 Hz, 1H), 2.40-2.14 (m, 2H), 1.36-1.23 (m, 2H), 1.10 (d, J=6.5 Hz, 3H).

### Example 9: 5-((3R,5S)-3-Amino-5-trifluoromethyl-piperidin-1-yl)-[1,7]naphthyridine-8-carbonitrile

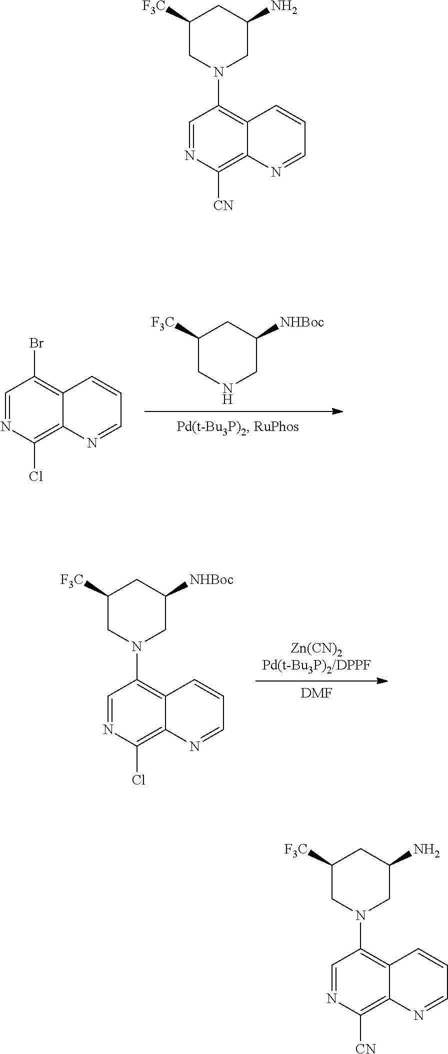

[(3R,5S)-1-(8-Chloro-[1,7]naphthyridin-5-yl)-5-trifluoromethyl-piperidin-3-yl]-carbamic acid tert-butyl ester: Within a 20 ml microwave vial was placed 5-Bromo-8-chloro-[1,7]naphthyridine (560 mg; 2.30 mmol; 1.0 eq.), ((3R,5S)-5-Trifluoromethyl-piperidin-3-yl)-carbamic acid tert-butyl ester (617 mg; 2.30 mmol; 1.0 eq.), RuPhos (53 mg; 0.11 mmol; 0.05 eq.) and dioxane (10 ml). The mixture was degased, then added 2-Methyl-propan-2-ol sodium (243 mg; 2.53 mmol; 1.10 eq.) and bis(tri-tert-butylphosphine)palladium(0) (58.8 mg; 0.11 mmol; 0.05 eq.), The resulted mixture was stirred at 90° C. for 4 hr, until the reaction was completed. The crude was purified by silica column to yield the title compound (300 mg, yield 30%). LC-MS (M+1)=431/433.

5-((3R,5S)-3-Amino-5-trifluoromethyl-piperidin-1-yl)-[1,7]naphthyridine-8-carbonitrile: Within 10 ml microwave tube, to a solution of [(3R,5S)-1-(8-Chloro-[1,7]naphthyridin-5-yl)-5-trifluoromethyl-piperidin-3-yl]-carbamic acid tert-butyl ester (170 mg; 0.39 mmol; 1.0 eq.) in DMF (1 ml) was added zinc cyanide (92 mg; 0.79 mmol; 2.0 eq.), and 1,1′-bis(diphenylphosphino)ferrocene (22 mg; 0.04 mmol; 0.10 eq.). The mixture was degased, and then added bis(tri-tert-butylphosphine)palladium(0) (10 mg; 0.02 mmol; 0.05 eq.). The tube was caped and microwaved at 150° C. for 2 hr, until there action was completed. The crude was purified by prep-HPLC, eluting with 20-70% ACN/water (contained 0.1% ammonia) to provide the title compound. LC-MS (M+1)=322. ¹H NMR (400 MHz, Methanol-d₄) δ 9.15 (dd, J=4.2, 1.6 Hz, 1H), 8.59 (dd, J=8.7, 1.6 Hz, 1H), 8.42 (s, 1H), 7.86 (dd, J=8.7, 4.2 Hz, 1H), 4.56 (s, 1H), 3.81-3.64 (m, 2H), 3.25 (td, J=10.9, 5.4 Hz, 1H), 3.07-2.90 (m, 1H), 2.72 (dd, J=11.7, 10.7 Hz, 1H), 2.38 (d, J=13.0 Hz, 1H), 1.43 (dd, J=12.3, 4.9 Hz, 2H), 1.36-1.21 (m, 1H).

### Example 10: (3R,5S)-1-(8-Ethoxy-[1,7]naphthyridin-5-yl)-5-trifluoromethyl-piperidin-3-ylamine

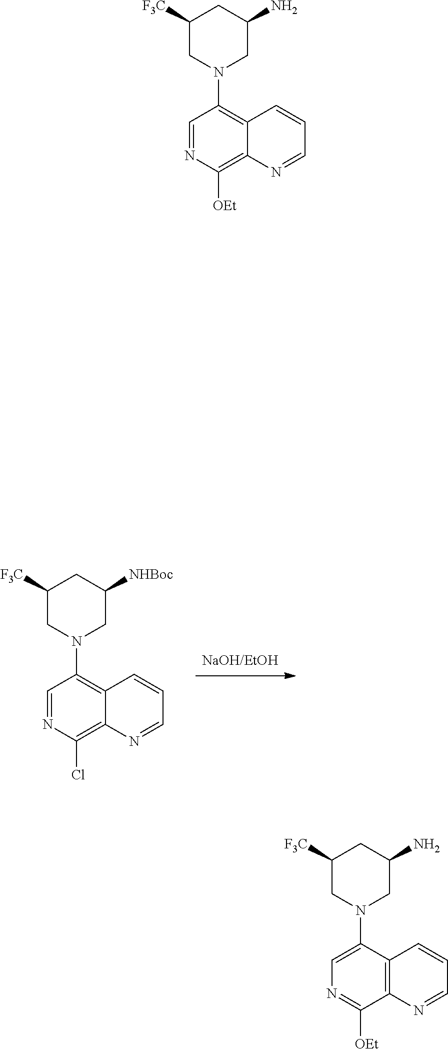 [SMILES: CCOc1ncc(N2C[C@H](N)C[C@H](C(F)(F)F)C2)c2cccnc12 ; CC(C)(C)OC(=O)N[C@@H]1C[C@H](C(F)(F)F)CN(c2cnc(Cl)c3ncccc23)C1 ; CCOc1ncc(N2C[C@H](N)C[C@H](C(F)(F)F)C2)c2cccnc12]

To a solution of [(3R,5S)-1-(8-Chloro-[1,7]naphthyridin-5-yl)-5-trifluoromethyl-piperidin-3-yl]-carbamic acid tert-butyl ester (300 mg; 0.01 mmol; 1.0 eq.) in Ethanol (0.4 ml) was added sodium hydroxide (2.0M aqueous) (1.0 ml; 2.0 mmol; 287.23 eq). The resulting mixture was stirred at 130° C. for 24 hr until the reaction was completed. The crude was purified by prep HPLC, eluting with 20-70% ACN/water (contained 0.1% ammonia), to provide the title compound. LC-MS (M+1)=341. ¹H NMR (400 MHz,) δ 8.11 (dd, J=4.2, 1.7 Hz, 1H), 7.73 (dd, J=8.5, 1.7 Hz, 1H), 7.05 (s, 1H), 7.03-6.97 (m, 1H), 3.81 (q, J=7.1 Hz, 2H), 2.66-2.54 (m, 2H), 2.52 (p, J=1.6 Hz, 2H), 2.38 (ddd, J=15.2, 10.6, 4.2 Hz, 1H), 2.16-2.01 (m, 2H), 1.74 (t, J=10.8 Hz, 1H), 1.52 (d, J=12.7 Hz, 1H), 0.73 (td, J=7.1, 1.8 Hz, 3H), 0.63-0.48 (m, 1H).

### Example 11: 4-{[(3R,5S)-5-Methyl-1-(8-trifluoromethyl-[1,7]naphthyridin-5-yl)-piperidin-3-ylamino]-methyl}-tetrahydro-pyran-4-ol

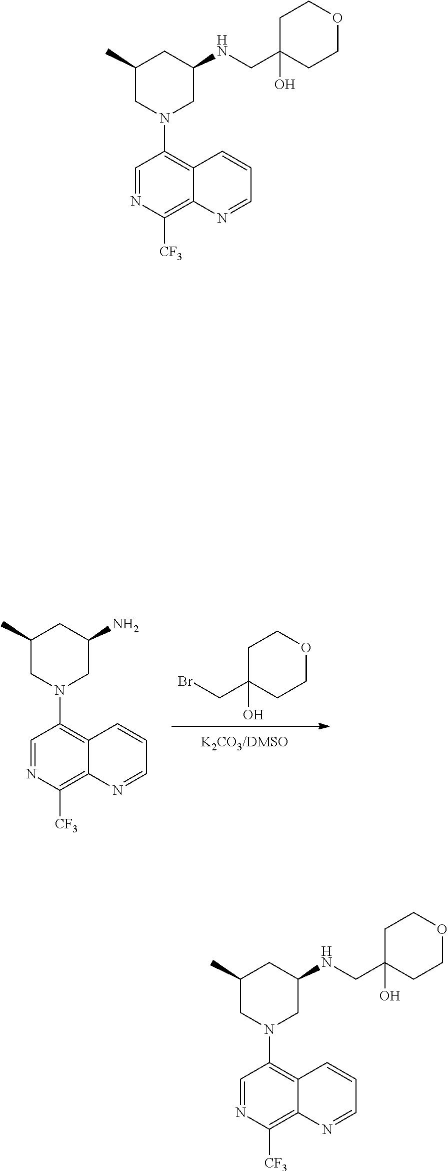 [SMILES: C[C@H]1C[C@@H](NCC2(O)CCOCC2)CN(c2cnc(C(F)(F)F)c3ncccc23)C1 ; C[C@H]1C[C@@H](N)CN(c2cnc(C(F)(F)F)c3ncccc23)C1 ; OC1(CBr)CCOCC1 ; C[C@H]1C[C@@H](NCC2(O)CCOCC2)CN(c2cnc(C(F)(F)F)c3ncccc23)C1]

A mixture of (3R,5S)-5-Methyl-1-(8-trifluoromethyl-[1,7]naphthyridin-5-yl)-piperidin-3-ylamine (50 mg; 0.16 mmol; 1.0 eq.), 4-Bromomethyl-tetrahydro-pyran-4-ol (47 mg; 0.24 mmol; 1.50 eq.), potassium carbonate (33 mg; 0.24 mmol; 1.50 eq.) in DMSO (1 ml) was stirred at 80° C. for 24 hr. The reaction mixture was cooled to rt. The crude was purified by prep HPLC, eluting with 20-70% ACN in water (contained 0.1% ammonia) to yield the title compound. LC-MS (M+1)=425. ¹H NMR (400 MHz, DMSO-d₆) δ 9.10 (d, J=3.9 Hz, 1H), 8.55 (d, J=8.6 Hz, 1H), 8.20 (s, 1H), 7.86 (dd, J=8.8, 4.1 Hz, 1H), 4.29 (d, J=12.2 Hz, 2H), 4.04 (dd, J=11.7, 3.4 Hz, 1H), 3.66-3.49 (m, 4H), 2.81 (dd, J=13.5, 8.2 Hz, 1H), 2.57 (d, J=4.5 Hz, 2H), 2.08 (d, J=12.1 Hz, 2H), 1.91 (s, 1H), 1.64-1.51 (m, 3H), 1.39 (d, J=13.3 Hz, 2H), 0.93 (m, J=7.3 Hz, 4H).

The following compound was synthesized in an analogous manner.

### Example 12: 8-[(3R,5S)-3-(1,1-Dioxo-1lambda6-thietan-3-ylamino)-5-methyl-piperidin-1-yl]-quinoxaline-5-carbonitrile

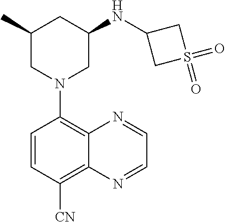 [SMILES: C[C@H]1C[C@@H](NC2CS(=O)(=O)C2)CN(c2ccc(C#N)c3nccnc23)C1]

The title compound was prepared from 8-((3R,5S)-3-Amino-5-methyl-piperidin-1-yl)-quinoxaline-5-carbonitrile hydrochloride (2) and 3-Bromo-thietane 1,1-dioxide. LC-MS (M+1)=372. ¹H NMR (400 MHz, DMSO-d₆) δ 9.0 (dd, J=27.4, 1.7 Hz, 2H), 8.17 (d, J=8.4 Hz, 1H), 7.21 (d, J=8.5 Hz, 1H), 4.52-4.24 (m, 3H), 4.13 (d, J=12.4 Hz, 1H), 3.92 (dt, J=13.0, 6.1 Hz, 2H), 3.76 (h, J=7.1 Hz, 1H), 2.89-2.69 (m, 1H), 2.59 (dp, J=11.7, 5.5 Hz, 3H), 2.10-1.98 (m, 1H), 1.88 (d, J=6.6 Hz, 1H), 1.04-0.73 (m, 4H).

### Example 13: (1,1-Dioxo-1lambda6-thietan-3-yl)-[(3R,5S)-5-methyl-1-(8-trifluoromethyl-quinolin-5-yl)-piperidin-3-yl]-amine

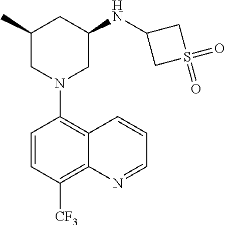 [SMILES: C[C@H]1C[C@@H](NC2CS(=O)(=O)C2)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine and 3-Bromo-thietane 1,1-dioxide. LC-MS (M+1)=414. ¹H NMR (400 MHz, DMSO-d₆) δ 9.01 (d, J=4.2 Hz, 1H), 8.47 (d, J=8.6 Hz, 1H), 8.05 (d, J=8.0 Hz, 1H), 7.67 (dd, J=8.7, 4.2 Hz, 1H), 7.20 (d, J=8.1 Hz, 1H), 4.32 (dt, J=21.0, 10.3 Hz, 2H), 3.99-3.87 (m, 2H), 3.75 (q, J=7.3 Hz, 1H), 3.51 (d, J=11.2 Hz, 1H), 2.95 (d, J=32.9 Hz, 1H), 2.59 (t, J=6.8 Hz, 1H), 2.38 (q, J=12.3, 11.8 Hz, 2H), 2.13-1.84 (m, 2H), 0.90 (dd, J=26.8, 9.1 Hz, 3H). 0.85-0.9 (m, 1H).

### Example 14: 8-{(3R,5S)-3-[(4-Hydroxy-tetrahydro-pyran-4-ylmethyl)-amino]-5-methyl-piperidin-1-yl}-quinoxaline-5-carbonitrile

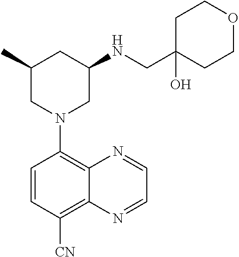 [SMILES: C[C@H]1C[C@@H](NCC2(O)CCOCC2)CN(c2ccc(C#N)c3nccnc23)C1]

The title compound was prepared from 8-((3R,5S)-3-Amino-5-methyl-piperidin-1-yl)-quinoxaline-5-carbonitrile and 4-Bromomethyl-tetrahydro-pyran-4-ol. LC-MS (M+1)=382. ¹H NMR (400 MHz, DMSO-d₆) δ 8.98 (dd, J=34.5, 1.7 Hz, 2H), 8.16 (d, J=8.4 Hz, 1H), 7.19 (d, J=8.5 Hz, 1H), 4.44 (d, J=12.0 Hz, 1H), 4.18 (d, J=14.2 Hz, 2H), 3.60 (d, J=12.6 Hz, 3H), 2.77 (s, 1H), 2.68-2.55 (m, 2H), 2.07 (d, J=13.0 Hz, 1H), 1.88 (s, 2H), 1.57 (d, J=13.0 Hz, 2H), 1.39 (d, J=13.3 Hz, 2H), 1.01-0.83 (m, 3H).

### Example 15: 3-[(3R,5S)-1-(8-Cyano-quinoxalin-5-yl)-5-methyl-piperidin-3-ylamino]-2-fluoro-2-methyl-propionic acid

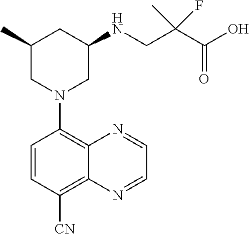 [SMILES: C[C@H]1C[C@@H](NCC(C)(F)C(=O)O)CN(c2ccc(C#N)c3nccnc23)C1]

The title compound was prepared from 8-((3R,5S)-3-Amino-5-methyl-piperidin-1-yl)-quinoxaline-5-carbonitrile hydrochloride and 3-Bromo-2-fluoro-2-methyl-propionic acid methyl ester. LC-MS (M+1)=372. ¹H NMR (400 MHz, Methanol-d₄) δ 8.92 (dd, J=12.8, 1.8 Hz, 2H), 8.10 (d, J=8.4 Hz, 1H), 7.30 (d, J=8.4 Hz, 1H), 4.39-4.14 (m, 3H), 4.07-3.62 (m, 3H), 2.97 (dd, J=11.7, 9.9 Hz, 1H), 2.74 (dd, J=12.4, 10.3 Hz, 1H), 2.19-2.0 (m, 2H), 1.63-1.37 (m, 4H), 1.04 (d, J=6.4 Hz, 3H).

### Example 16: 8-[(3R,5S)-3-(2-Hydroxy-2-methyl-propylamino)-5-methyl-piperidin-1-yl]-quinoxaline-5-carbonitrile formic acid

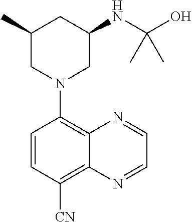 [SMILES: C[C@H]1C[C@@H](NC(C)(C)O)CN(c2ccc(C#N)c3nccnc23)C1]

The title compound was prepared from 8-((3R,5S)-3-Amino-5-methyl-piperidin-1-yl)-quinoxaline-5-carbonitrile hydrochloride and 1-bromo-2-methylpropan-2-ol. LC-MS (M+1)=340. H NMR (400 MHz, DMSO-d₆) δ 9.02 (d, J=1.8 Hz, 1H), 8.94 (d, J=1.8 Hz, 1H), 8.24 (s, 1H), 8.17 (d, J=8.5 Hz, 1H), 7.20 (d, J=8.5 Hz, 1H), 4.45 (dd, J=11.8, 2.7 Hz, 1H), 4.18 (dd, J=13.1, 3.5 Hz, 1H), 2.79 (dq, J=10.8, 5.3, 3.8 Hz, 1H), 2.62 (td, J=11.7, 4.9 Hz, 2H), 2.55-2.52 (m, 2H), 2.07 (d, J=12.3 Hz, 1H), 1.88 (dq, J=10.8, 7.0 Hz, 1H), 1.09 (s, 6H), 1.0-0.80 (m, 4H).

### Example 17: 2-Methyl-1-[(3R,5S)-5-methyl-1-(8-trifluoromethyl-quinolin-5-yl)-piperidin-3-ylamino]-propan-2-ol

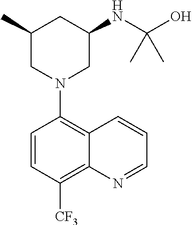 [SMILES: C[C@H]1C[C@@H](NC(C)(C)O)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride and 1-bromo-2-methylpropan-2-ol. LC-MS (M+1)=382. ¹H NMR (400 MHz, DMSO-d₆) δ 9.01 (dd, J=4.1, 1.7 Hz, 1H), 8.48 (dd, J=8.6, 1.8 Hz, 1H), 8.05 (d, J=8.1 Hz, 1H), 7.66 (dd, J=8.6, 4.1 Hz, 1H), 7.20 (d, J=8.0 Hz, 1H), 4.12 (s, 1H), 3.54 (d, J=10.9 Hz, 1H), 2.88 (d, J=10.8 Hz, 1H), 2.64-2.22 (m, 5H), 2.18-1.93 (m, 2H), 1.52 (s, 1H), 1.07 (s, 6H), 1.03-0.55 (m, 4H).

### Examples 18: 2-{[(3R,5S)-5-methyl-1-[8-(trifluoromethyl)quinolin-5-yl]piperidin-3-yl]amino}-1-(morpholin-4-yl)ethan-1-one

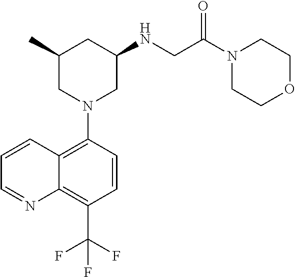 [SMILES: C[C@H]1C[C@@H](NCC(=O)N2CCOCC2)CN(c2ccc(C(F)(F)F)c3ncccc23)C1]

The title compound was prepared from (3R,5S)-5-methyl-1-[8-(trifluoromethyl)quinolin-5-yl]piperidin-3-aminium trifluoroacetate and 4-(bromoacetyl)morpholine. MS: 437 [M+H]⁺. ¹H NMR (400 MHz, CDCl₃) δ 9.04 (dd, J=4.2, 1.7 Hz, 1H), 8.42 (dd, J=8.6, 1.8 Hz, 1H), 7.97 (d, J=8.0 Hz, 1H), 7.47 (dd, J=8.6, 4.2 Hz, 1H), 7.08 (d, J=7.9 Hz, 1H), 3.73-3.60 (m, 6H), 3.55-3.52 (m, 3H), 3.40 (t, J=4.8 Hz, 2H), 3.36-3.30 (m, 1H), 3.10-3.02 (m, 1H), 2.66 (dd, J=10.6, 7.6 Hz, 1H), 2.37 (t, J=11.4 Hz, 1H), 2.25-2.17 (m, 1H), 1.09 (q, J=11.9 Hz, 1H), 1.0 (d, J=6.6 Hz, 3H).

### Examples 19: N-(2-{[(3R,5S)-5-methyl-1-[8-(trifluoromethyl)quinolin-5-yl]piperidin-3-yl]amino}ethyl)aminosulfonamide

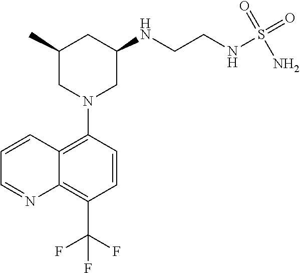 [SMILES: C[C@H]1C[C@@H](NCCNS(N)(=O)=O)CN(c2ccc(C(F)(F)F)c3ncccc23)C1]

The title compound was prepared from (3R,5S)-5-methyl-1-[8-(trifluoromethyl)quinolin-5-yl]piperidin-3-aminium trifluoroacetate and [(2-bromoethyl)sulfamoyl]amine. MS: 432 [M+H]⁺. ¹H NMR (400 MHz, CDCl₃) δ 9.04 (d, J=4.1 Hz, 1H), 8.44 (d, J=8.6 Hz, 1H), 7.96 (d, J=7.9 Hz, 1H), 7.49 (dd, J=8.6, 4.2 Hz, 1H), 7.05 (d, J=8.0 Hz, 1H), 3.81-3.74 (m, 2H), 3.31 (d, J=11.6 Hz, 1H), 3.13-3.07 (m, 2H), 2.93-2.84 (m, 2H), 2.53 (d, J=12.0 Hz, 1H), 2.37 (t, J=11.3 Hz, 1H), 2.28 (d, J=12.4 Hz, 1H), 2.18-2.08 (m, 1H), 1.12-0.94 (m, 4H).

### Examples 20: N-(2-{[(3R,5S)-5-methyl-1-[8-(trifluoromethyl)quinolin-5-yl]piperidin-3-yl]amino}ethyl)methanesulfonamide

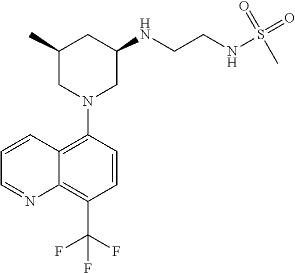 [SMILES: C[C@H]1C[C@@H](NCCNS(C)(=O)=O)CN(c2ccc(C(F)(F)F)c3ncccc23)C1]

The title compound was prepared from (3R,5S)-5-methyl-1-[8-(trifluoromethyl)quinolin-5-yl]piperidin-3-aminium trifluoroacetate and N-(2-bromoethyl)methanesulfonamide. MS: 431 [M+H]⁺. ¹H NMR (400 MHz, CDCl₃) δ 9.09-9.01 (m, 1H), 8.43 (dd, J=8.5, 1.5 Hz, 1H), 7.97 (d, J=8.0 Hz, 1H), 7.48 (dd, J=8.6, 4.2 Hz, 1H), 7.07 (d, J=7.9 Hz, 1H), 3.56 (d, J=11.2 Hz, 1H), 3.33 (d, J=11.7 Hz, 1H), 3.21 (t, J=5.6 Hz, 2H), 3.10-2.83 (m, 6H), 2.44 (t, J=10.7 Hz, 1H), 2.36 (t, J=11.3 Hz, 1H), 2.19 (d, J=12.7 Hz, 1H), 2.13-2.03 (m, 1H), 0.99 (d, J=6.6 Hz, 3H), 0.98-0.87 (m, 1H).

### Examples 21: 8-[(3R,5S)-3-[(3-methanesulfonylpropyl)amino]-5-methylpiperidin-1-yl]quinoxaline-5-carbonitrile methanesulfonamide

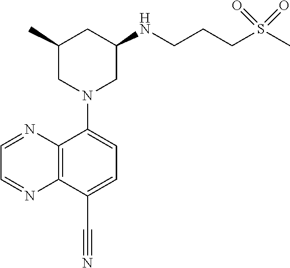 [SMILES: C[C@H]1C[C@@H](NCCCS(C)(=O)=O)CN(c2ccc(C#N)c3nccnc23)C1]

The title compound was prepared from (3R,5S)-5-methyl-1-[8-(trifluoromethyl)quinolin-5-yl]piperidin-3-aminium trifluoroacetate and 1-bromo-3-methanesulfonylpropane. MS: 388 [M+H]⁺. ¹H NMR (400 MHz, DMSO-d₆) δ 9.02 (d, J=1.8 Hz, 1H), 8.94 (d, J=1.8 Hz, 1H), 8.16 (d, J=8.4 Hz, 1H), 7.19 (d, J=8.5 Hz, 1H), 4.46 (d, J=11.7 Hz, 1H), 4.14 (d, J=12.2 Hz, 1H), 3.30 (d, J=1.2 Hz, 2H), 3.16 (dd, J=6.4, 4.0 Hz, 2H), 2.96 (s, 3H), 2.86-2.67 (m, 2H), 2.65-2.52 (m, 2H), 2.50 (p, J=1.8 Hz, 4H), 2.05 (d, J=12.5 Hz, 1H), 1.95-1.66 (m, 3H), 0.93 (t, J=6.6 Hz, 4H).

### Example 22: 3-[(3R,5S)-5-Methyl-1-(8-trifluoromethyl-quinoxalin-5-yl)-piperidin-3-ylamino]-propionamide

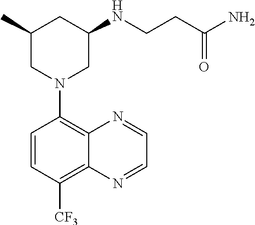 [SMILES: C[C@H]1C[C@@H](NCCC(N)=O)CN(c2ccc(C(F)(F)F)c3nccnc23)C1]

The title compound was prepared from (3R,5S)-5-Methyl-1-(8-trifluoromethyl-quinoxalin-5-yl)-piperidin-3-ylamine hydrochloride and 3-Bromo-propionamide. LC-MS (M+1)=382. H NMR (400 MHz, DMSO-d₆) δ 8.98 (dd, J=21.1, 1.8 Hz, 2H), 8.04 (d, J=8.4 Hz, 1H), 7.34 (s, 1H), 7.20 (d, J=8.4 Hz, 1H), 6.73 (s, 1H), 4.28 (d, J=11.4 Hz, 1H), 4.02 (d, J=11.8 Hz, 1H), 2.80 (d, J=6.4 Hz, 3H), 2.48-2.43 (m, 1H), 2.21 (t, J=6.8 Hz, 2H), 2.05 (d, J=12.5 Hz, 1H), 1.91 (s, 1H), 1.64 (d, J=6.5 Hz, 1H), 1.01-0.76 (m, 4H).

### Example 23: N-{2-[(3R,5S)-5-Methyl-1-(8-trifluoromethyl-quinoxalin-5-yl)-piperidin-3-ylamino]-ethyl}-methanesulfonamide

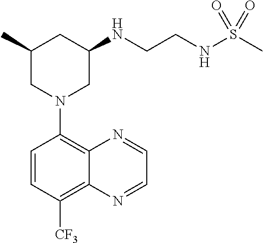

The title compound was prepared from (3R,5S)-5-Methyl-1-(8-trifluoromethyl-quinoxalin-5-yl)-piperidin-3-ylamine hydrochloride and N-(2-Bromo-ethyl)-methanesulfonamide. LC-MS (M+1)=432. ¹H NMR (400 MHz, DMSO-d₆) δ 8.98 (dd, J=21.3, 1.8 Hz, 2H), 8.04 (d, J=8.4 Hz, 1H), 7.19 (dd, J=8.5, 4.1 Hz, 1H), 6.92 (s, 1H), 4.30 (d, J=11.0 Hz, 1H), 4.07-3.92 (m, 1H), 3.03 (t, J=6.5 Hz, 2H), 2.91 (s, 3H), 2.83 (d, J=10.7 Hz, 1H), 2.74 (t, J=5.7 Hz, 2H), 2.48-2.43 (m, 2H), 2.06 (d, J=13.1 Hz, 1H), 1.99-1.83 (m, 1H), 1.75 (s, 1H), 1.0-0.80 (m, 4H).

### Example 24: N-{2-[(3R,5S)-5-Methyl-1-(8-trifluoromethyl-quinoxalin-5-yl)-piperidin-3-ylamino]-ethyl}-methanesulfonamide

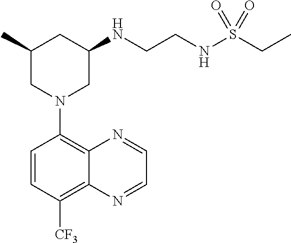 [SMILES: CCS(=O)(=O)NCCN[C@@H]1C[C@H](C)CN(c2ccc(C(F)(F)F)c3nccnc23)C1]

The title compound was prepared from (3R,5S)-5-Methyl-1-(8-trifluoromethyl-quinoxalin-5-yl)-piperidin-3-ylamine hydrochloride and N-(2-bromoethyl)ethane-1-sulfonamide. LC-MS (M+1)=446. ¹H NMR (400 MHz, DMSO-d₆) δ 8.99 (d, J=22.3 Hz, 2H), 8.04 (d, J=8.3 Hz, 1H), 7.20 (d, J=8.4 Hz, 1H), 6.96 (s, 1H), 4.31 (d, J=11.7 Hz, 1H), 4.0 (d, J=11.9 Hz, 1H), 3.08-2.93 (m, 4H), 2.83 (d, J=11.2 Hz, 1H), 2.72 (s, 2H), 2.45 (d, J=11.1 Hz, 2H), 2.05 (d, J=12.2 Hz, 1H), 1.91 (s, 1H), 1.75 (s, 1H), 1.19 (td, J=7.3, 2.0 Hz, 3H), 1.01-0.78 (m, 4H).

### Example 25: N-{2-[(3R,5S)-5-Methyl-1-(8-trifluoromethyl-quinoxalin-5-yl)-piperidin-3-ylamino]-ethyl}-acetamide formic acid

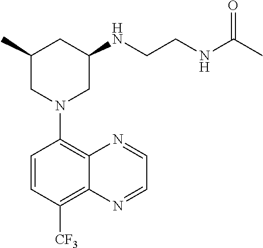 [SMILES: CC(=O)NCCN[C@@H]1C[C@H](C)CN(c2ccc(C(F)(F)F)c3nccnc23)C1]

The title compound was prepared from (3R,5S)-5-Methyl-1-(8-trifluoromethyl-quinoxalin-5-yl)-piperidin-3-ylamine hydrochloride and N-(2-Chloro-ethyl)-acetamide. LC-MS (M+1)=442. ¹H NMR (400 MHz, Methanol-d₄) δ 9.0-8.87 (m, 2H), 8.54 (s, 1H), 8.08 (t, J=6.5 Hz, 1H), 7.30 (t, J=9.6 Hz, 1H), 4.31 (d, J=11.8 Hz, 1H), 4.17 (q, J=12.3 Hz, 1H), 3.97 (d, J=12.0 Hz, 1H), 3.84 (d, J=12.3 Hz, 1H), 3.77 (dt, J=10.7, 5.2 Hz, 2H), 3.57 (d, J=5.4 Hz, 1H), 3.45 (d, J=5.6 Hz, 1H), 2.79 (t, J=11.1 Hz, 1H), 2.74-2.59 (m, 1H), 2.37 (d, J=2.2 Hz, 2H), 2.23 (q, J=31.3, 22.4 Hz, 2H), 1.46 (q, J=11.9 Hz, 1H), 1.30 (q, J=11.9 Hz, 1H), 1.16-0.99 (m, 3H).

### Example 26: 4-{[(3R,5S)-5-Methyl-1-(8-trifluoromethyl-quinoxalin-5-yl)-piperidin-3-ylamino]-methyl}-tetrahydro-pyran-4-ol

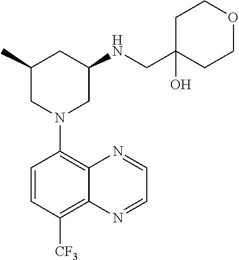 [SMILES: C[C@H]1C[C@@H](NCC2(O)CCOCC2)CN(c2ccc(C(F)(F)F)c3nccnc23)C1]

The title compound was prepared from (3R,5S)-5-Methyl-1-(8-trifluoromethyl-quinoxalin-5-yl)-piperidin-3-ylamine hydrochloride and N-(2-Chloro-ethyl)-acetamide. LC-MS (M+1)=425. ¹H NMR (400 MHz, DMSO-d₆) δ 8.98 (d, J=22.3 Hz, 2H), 8.05 (d, J=8.4 Hz, 1H), 7.20 (d, J=8.4 Hz, 1H), 4.29 (d, J=12.2 Hz, 2H), 4.04 (dd, J=11.7, 3.4 Hz, 1H), 3.69-3.48 (m, 4H), 2.81 (dd, J=13.5, 8.2 Hz, 2H), 2.57 (d, J=4.5 Hz, 2H), 2.08 (d, J=12.1 Hz, 1H), 1.91 (s, 1H), 1.61-1.45 (m, 2H), 1.39 (d, J=13.3 Hz, 2H), 0.93 (t, J=7.3 Hz, 4H).

### Example 27: 1-{2-[(3R,5S)-5-Methyl-1-(8-trifluoromethyl-quinolin-5-yl)-piperidin-3-ylamino]-ethyl}-imidazolidin-2-one

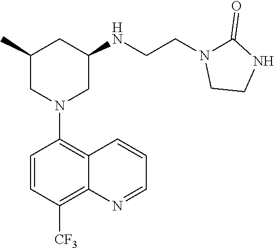 [SMILES: C[C@H]1C[C@@H](NCCN2CCNC2=O)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride and 1-(2-Bromo-ethyl)-imidazolidin-2-one. LC-MS (M+1)=422. ¹H NMR (400 MHz, DMSO-d₆) δ 9.0 (d, J=4.1 Hz, 1H), 8.49 (d, J=8.6 Hz, 1H), 8.04 (d, J=8.0 Hz, 1H), 7.67 (dd, J=8.6, 4.2 Hz, 1H), 7.20 (d, J=8.1 Hz, 1H), 6.22 (s, 1H), 3.60-3.47 (m, 1H), 3.34 (dd, J=8.9, 6.5 Hz, 2H), 3.20 (t, J=7.9 Hz, 2H), 3.09 (hept, J=6.6 Hz, 2H), 2.95 (s, 1H), 2.69 (s, 2H), 2.37 (td, J=11.1, 7.1 Hz, 2H), 2.14-1.85 (m, 2H), 1.62 (s, 1H), 0.94 (d, J=6.5 Hz, 3H), 0.84 (t, J=11.7 Hz, 1H).

### Example 28: 5-{[(3R,5S)-5-Methyl-1-(8-trifluoromethyl-quinoxalin-5-yl)-piperidin-3-ylamino]-methyl}-pyrrolidin-2-one

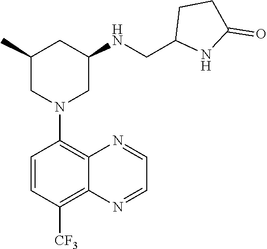 [SMILES: C[C@H]1C[C@@H](NCC2CCC(=O)N2)CN(c2ccc(C(F)(F)F)c3nccnc23)C1]

The title compound was prepared from (3R,5S)-5-Methyl-1-(8-trifluoromethyl-quinoxalin-5-yl)-piperidin-3-ylamine hydrochloride and 5-Bromomethyl-pyrrolidin-2-one. LC-MS (M+1)=408. ¹H NMR (400 MHz, Methanol-d₄) δ 9.05-8.89 (m, 2H), 8.42 (s, 1H), 8.08 (t, J=8.5 Hz, 1H), 7.29 (d, J=8.6 Hz, 1H), 4.50 (d, J=11.2 Hz, 1H), 3.94 (d, J=14.0 Hz, 2H), 3.75 (m, 1H), 3.37 (d, J=11.7 Hz, 1H), 3.13 (d, J=4.7 Hz, 1H), 2.99 (dd, J=13.0, 7.6 Hz, 1H), 2.71 (t, J=11.0 Hz, 1H), 2.61 (q, J=11.9 Hz, 1H), 2.37 (td, J=23.1, 19.0, 10.1 Hz, 3H), 2.13 (s, 1H), 1.91 (d, J=11.4 Hz, 1H), 1.25 (dt, J=46.9, 12.0 Hz, 1H), 1.09 (dd, J=9.7, 7.2 Hz, 3H).

### Example 29: 3-{2-[(3R,5S)-5-Methyl-1-(8-trifluoromethyl-quinolin-5-yl)-piperidin-3-ylamino]-ethyl}-oxazolidin-2-one

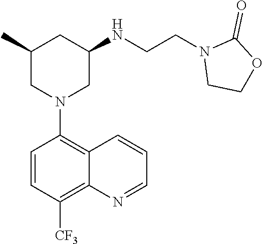 [SMILES: C[C@H]1C[C@@H](NCCN2CCOC2=O)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride and 3-(2-Bromo-ethyl)-oxazolidin-2-one. LC-MS (M+1)=423. ¹H NMR (400 MHz, DMSO-d₆) δ 9.01 (d, J=3.9 Hz, 1H), 8.49 (dd, J=8.6, 1.6 Hz, 1H), 8.05 (d, J=8.1 Hz, 1H), 7.66 (dd, J=8.6, 4.2 Hz, 1H), 7.20 (d, J=8.0 Hz, 1H), 4.23 (t, J=8.0 Hz, 2H), 3.55 (q, J=7.0, 5.7 Hz, 2H), 3.29 (s, 1H), 3.22 (td, J=6.5, 2.9 Hz, 2H), 2.95 (s, 1H), 2.82-2.65 (m, 2H), 2.38 (td, J=11.1, 5.2 Hz, 2H), 2.06 (dd, J=32.7, 11.4 Hz, 2H), 1.75 (d, J=6.6 Hz, 1H), 0.99-0.87 (m, 3H), 0.83 (d, J=11.7 Hz, 1H).

### Example 30: 3-{2-[(3R,5S)-5-Methyl-1-(8-trifluoromethyl-quinolin-5-yl)-piperidin-3-ylamino]-ethyl}-pyrrolidin-2-one

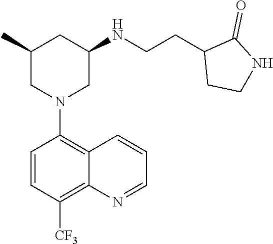 [SMILES: C[C@H]1C[C@@H](NCCC2CCNC2=O)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride and 3-(2-Bromo-ethyl)-pyrrolidin-2-one. LC-MS (M+1)=421. ¹H NMR (400 MHz, DMSO-d₆) δ 9.01 (d, J=4.1 Hz, 1H), 8.48 (d, J=8.4 Hz, 1H), 8.05 (d, J=8.1 Hz, 1H), 7.66 (dd, J=8.6, 4.2 Hz, 1H), 7.49 (s, 1H), 7.20 (d, J=8.1 Hz, 1H), 3.53 (d, J=11.3 Hz, 1H), 3.21-3.04 (m, 2H), 2.92 (s, 1H), 2.64 (s, 2H), 2.44-2.30 (m, 2H), 2.25 (dd, J=9.1, 4.6 Hz, 1H), 2.21-1.91 (m, 3H), 1.89-1.73 (m, 1H), 1.61 (d, J=9.7 Hz, 2H), 1.32 (dt, J=14.2, 7.9 Hz, 1H), 0.94 (d, J=6.5 Hz, 3H), 0.89-0.76 (m, 1H).

### Example 31: 3-{2-[(3R,5S)-5-Methyl-1-(8-trifluoromethyl-quinoxalin-5-yl)-piperidin-3-ylamino]-ethyl}-oxazolidin-2-one formic acid

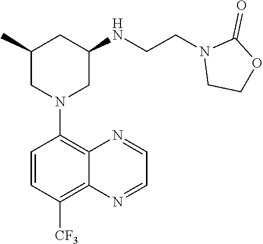 [SMILES: C[C@H]1C[C@@H](NCCN2CCOC2=O)CN(c2ccc(C(F)(F)F)c3nccnc23)C1]

The title compound was prepared from (3R,5S)-5-Methyl-1-(8-trifluoromethyl-quinoxalin-5-yl)-piperidin-3-ylamine hydrochloride and 3-(2-Bromo-ethyl)-oxazolidin-2-one. LC-MS (M+1)=424. ¹H NMR (400 MHz, Methanol-d₄) δ 8.94 (d, J=6.4 Hz, 2H), 8.47 (s, 1H), 8.07 (d, J=8.4 Hz, 1H), 7.29 (d, J=8.3 Hz, 1H), 4.51 (d, J=11.6 Hz, 1H), 4.41 (t, J=8.1 Hz, 2H), 3.94 (d, J=11.8 Hz, 1H), 3.71 (t, J=8.3 Hz, 2H), 3.57 (s, 2H), 3.46 (s, 1H), 3.19 (d, J=6.2 Hz, 2H), 2.74 (t, J=11.0 Hz, 1H), 2.61 (t, J=11.5 Hz, 1H), 2.31 (d, J=12.5 Hz, 1H), 2.12 (s, 1H), 1.18 (q, J=12.0 Hz, 1H), 1.08 (d, J=6.5 Hz, 3H).

### Example 32: 3-[(3R,5S)-5-Methyl-1-(8-trifluoromethyl-quinolin-5-yl)-piperidin-3-ylamino]-propane-1-sulfonic acid methylamide

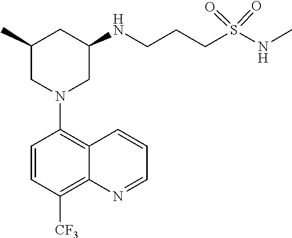

The title compound was prepared from (3R,5S)-5-Methyl-1-(8-trifluoromethyl-quinolin-5-yl)-piperidin-3-ylamine and 3-Chloro-propane-1-sulfonic acid methylamide. LC-MS (M+1)=445. ¹H NMR (400 MHz, DMSO-d₆) δ 9.01 (s, 1H), 8.48 (d, J=8.6 Hz, 1H), 8.05 (d, J=8.1 Hz, 1H), 7.66 (d, J=10.3 Hz, 1H), 7.20 (d, J=7.9 Hz, 1H), 6.86 (s, 1H), 3.53 (d, J=11.2 Hz, 1H), 3.04 (t, J=7.9 Hz, 1H), 2.90 (s, 1H), 2.68 (s, 2H), 2.56 (t, J=2.7 Hz, 2H), 2.38 (d, J=9.2 Hz, 3H), 2.17-1.90 (m, 3H), 1.87 (s, 1H), 1.83-1.64 (m, 2H), 1.03-0.91 (m, 3H), 0.91-0.79 (m, 1H).

### Example 33: 2-Methyl-4-[(3R,5S)-5-methyl-1-(8-trifluoromethyl-quinolin-5-yl)-piperidin-3-ylamino]-butan-2-ol

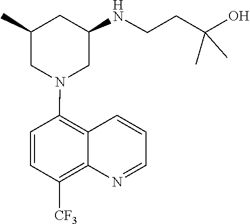 [SMILES: C[C@H]1C[C@@H](NCCC(C)(C)O)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride and 4-Bromo-2-methyl-butan-2-ol. LC-MS (M+1)=396. ¹H NMR (400 MHz, Methanol-d₄) δ 9.0-8.83 (m, 1H), 8.61 (d, J=8.6 Hz, 1H), 8.05 (d, J=8.1 Hz, 1H), 7.61 (dt, J=6.4, 3.0 Hz, 1H), 7.25 (d, J=8.2 Hz, 1H), 3.64 (d, J=11.3 Hz, 1H), 3.40 (s, 1H), 3.08 (d, J=11.3 Hz, 1H), 2.87 (p, J=9.8, 8.3 Hz, 2H), 2.48 (dt, J=23.2, 11.1 Hz, 2H), 2.24 (d, J=12.7 Hz, 1H), 2.13 (s, 2H), 1.70 (t, J=7.6 Hz, 2H), 1.23 (s, 6H), 1.08-0.77 (m, 4H).

### Example 34: 3-[(3R,5S)-1-(8-Cyano-quinoxalin-5-yl)-5-methyl-piperidin-3-ylamino]-propane-1-sulfonic acid amide

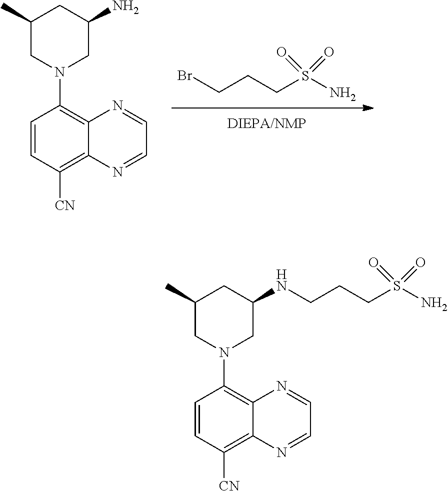 [SMILES: C[C@H]1C[C@@H](N)CN(c2ccc(C#N)c3nccnc23)C1 ; NS(=O)(=O)CCCBr ; C[C@H]1C[C@@H](NCCCS(N)(=O)=O)CN(c2ccc(C#N)c3nccnc23)C1]

To a 10 ml microwave tube, was placed 8-((3R,5S)-3-Amino-5-methyl-piperidin-1-yl)-quinoxaline-5-carbonitrile (50 mg; 0.19 mmol; 1.0 eq.), 3-Bromo-propane-1-sulfonic acid amide (56 mg; 0.28 mmol; 1.50 eq.), Ethyl-diisopropyl-amine (0.08 ml; 0.47 mmol; 2.50 eq.) and NMP (1 ml). The mixture was stirred at 80° C. for 4 hr. The crude was purified by prep HPLC, eluting with 10-60% ACN/water (contained 0.1% ammonia) to provide the title compound (33 mg, yield: 45%). LC-MS (M+1)=389. ¹H NMR (400 MHz, DMSO-d₆) δ 8.98 (dd, J=31.1, 1.9 Hz, 2H), 8.16 (d, J=8.3 Hz, 1H), 7.18 (d, J=8.4 Hz, 1H), 6.75 (s, 2H), 4.50-4.36 (m, 1H), 4.20-4.09 (m, 1H), 3.03 (dd, J=9.1, 6.5 Hz, 2H), 2.85-2.63 (m, 3H), 2.58 (dt, J=15.5, 11.4 Hz, 2H), 2.05 (d, J=12.6 Hz, 1H), 1.83 (p, J=7.0 Hz, 3H), 1.01-0.85 (m, 3H).

The following compound was synthesized in an analogous manner.

### Example 35: 5-[(3R,5S)-3-(2,3-Dihydroxy-propylamino)-5-methyl-piperidin-1-yl]-naphthyridine-8-carbonitrile

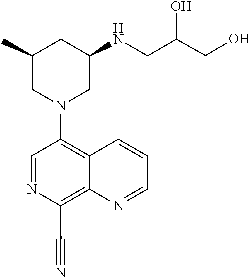 [SMILES: C[C@H]1C[C@@H](NCC(O)CO)CN(c2cnc(C#N)c3ncccc23)C1]

The title compound was prepared from 5-((3R,5S)-3-Amino-5-methyl-piperidin-1-yl)-[1,7]naphthyridine-8-carbonitrile and 3-Bromo-propane-1,2-diol. MS: 342.3 [M+H]⁺. 1H NMR (400 MHz, DMSO-d₆) δ 9.17 (d, J=4.1 Hz, 1H), 8.48 (d, J=8.6 Hz, 1H), 8.36 (s, 1H), 7.86 (dd, J=8.7, 4.2 Hz, 1H), 5.75 (s, 3H), 3.81-3.72 (m, 2H), 3.57-3.48 (m, 3H), 2.92 (s, 1H), 2.73 (dd, J=11.9, 4.5 Hz, 1H), 2.60 (q, J=10.6 Hz, 2H), 2.54 (s, 1H), 2.09 (d, J=12.6 Hz, 1H), 1.99 (s, 1H), 0.95 (d, J=6.6 Hz, 3H).

### Example 36: N-Hydroxy-3-[(3R,5S)-5-methyl-1-(8-trifluoromethyl-quinolin-5-yl)-piperidin-3-ylamino]-propionamide

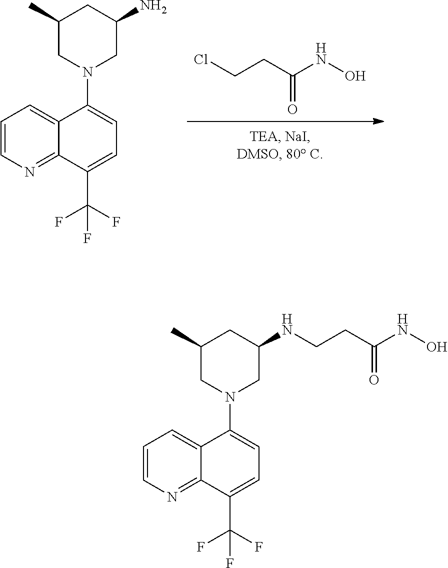 [SMILES: C[C@H]1C[C@@H](N)CN(c2ccc(C(F)(F)F)c3ncccc23)C1 ; O=C(CCCl)NO ; C[C@H]1C[C@@H](NCCC(=O)NO)CN(c2ccc(C(F)(F)F)c3ncccc23)C1]

A mixture of (3R,5S)-5-Methyl-1-(8-trifluoromethyl-quinolin-5-yl)-piperidin-3-ylamine (55.0 mg; 0.18 mmol; 1.0 eq.), 3-Chloro-N-hydroxy-propionamide (32.95 mg; 0.27 mmol; 1.50 eq.) and Triethyl-amine (44.98 mg; 0.44 mmol; 2.50 eq.) in DMSO (1 mL) stirred at 80° C. overnight. Once complete, the reaction was purified by prep HPLC with an acetonitrile/water (0.1% NH₄OH modified) gradient to afford the title compound (4.50 mg; 0.01 mmol; 6.4%). MS: 397.1 [M+H]⁺. 1H NMR (400 MHz, DMSO-d₆) δ 9.01 (dd, J=4.1, 1.7 Hz, 1H), 8.48 (dt, J=10.1, 3.1 Hz, 1H), 8.08-8.02 (m, 1H), 7.68-7.63 (m, 1H), 7.34 (s, 1H), 7.19 (d, J=8.1 Hz, 1H), 3.57-3.48 (m, 1H), 3.32 (s, 2H), 2.99-2.89 (m, 1H), 2.85-2.72 (m, 1H), 2.37 (td, J=10.9, 4.7 Hz, 2H), 2.20 (t, J=6.8 Hz, 1H), 2.12-1.95 (m, 2H), 0.93 (dd, J=6.5, 3.5 Hz, 3H), 0.86 (q, J=11.5 Hz, 1H).

### Example 37: N-{2-[(3R,5S)-5-Methyl-1-(8-trifluoromethyl-quinolin-5-yl)-piperidin-3-ylamino]-ethyl}-acetamide formic acid

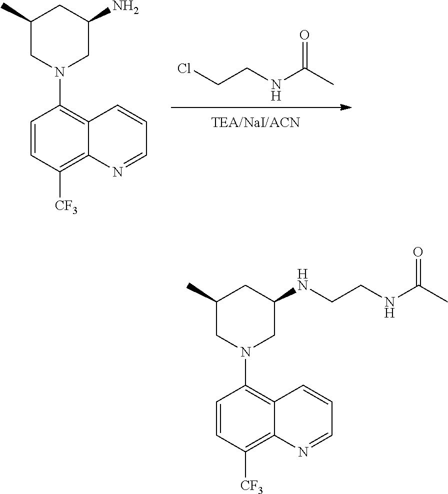

Within a 10 ml microwave tube, a mixture of (3R,5S)-5-Methyl-1-(8-trifluoromethyl-quinolin-5-yl)-piperidin-3-ylamine hydrochloride (300 mg; 0.87 mmol; 1.0 eq.), N-(2-Chloro-ethyl)-acetamide (166 mg; 1.30 mmol; 1.50 eq.), sodium iodide (39.01 mg; 0.26 mmol; 0.30 eq.) and triethyl-amine (0.30 ml; 2.17 mmol; 2.50 eq.) in ACN (3 ml) was stirred at 80° C. for 72 hr, until the reaction was completed. The reaction mixture was cooled to rt. The crude was purified by prep HPLC, eluting with 20-60% CAN/water (contained 0.1% ammonia), to yield the title compound (150 mg, yield 39%). LC-MS (M+1)=372. ¹H NMR (400 MHz, DMSO-d₆) δ 9.03 (d, J=4.1 Hz, 1H), 8.58 (dd, J=24.6, 8.7 Hz, 1H), 8.42 (s, 1H), 8.09 (d, J=8.1 Hz, 1H), 7.68 (dd, J=8.7, 4.2 Hz, 1H), 7.24 (d, J=8.1 Hz, 1H), 4.18 (d, J=25.1 Hz, 1H), 4.0 (s, 1H), 3.69-3.46 (m, 4H), 2.79 (t, J=10.9 Hz, 1H), 2.64 (d, J=10.4 Hz, 1H), 2.40 (t, J=9.8 Hz, 1H), 2.33 (s, 1H), 2.24 (s, 1H), 2.20-2.06 (m, 2H), 1.33 (d, J=12.4 Hz, 1H), 1.16 (q, J=13.2, 12.3 Hz, 1H), 0.97 (d, J=6.3 Hz, 3H).

The following compound was synthesized in an analogous manner.

### Example 38: 3-[(3R,5S)-1-(8-Cyano-quinolin-5-yl)-5-trifluoromethyl-piperidin-3-ylamino]-propane-1-sulfonic acid amide

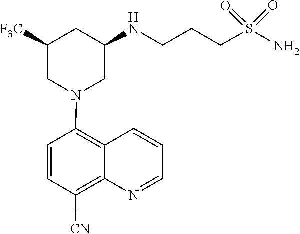 [SMILES: N#Cc1ccc(N2C[C@H](NCCCS(N)(=O)=O)C[C@H](C(F)(F)F)C2)c2cccnc12]

The title compound was prepared from 5-((3R,5S)-3-Amino-5-trifluoromethyl-piperidin-1-yl)-quinoline-8-carbonitrile and 3-Bromo-propane-1-sulfonic acid amide. LC-MS (M+1)=442. 1H NMR (400 MHz, DMSO-d₆) δ 9.06 (d, J=4.2 Hz, 1H), 8.51 (d, J=8.5 Hz, 1H), 8.25 (d, J=8.0 Hz, 1H), 7.71 (dd, J=8.9, 4.2 Hz, 1H), 7.31 (d, J=8.1 Hz, 1H), 6.74 (s, 2H), 3.57 (t, J=13.5 Hz, 3H), 3.02 (dd, J=9.1, 6.7 Hz, 4H), 2.88 (t, J=11.4 Hz, 1H), 2.70 (d, J=6.8 Hz, 2H), 2.29 (d, J=12.2 Hz, 1H), 2.03 (d, J=47.9 Hz, 1H), 1.81 (t, J=7.8 Hz, 2H), 1.25 (q, J=12.0 Hz, 1H), 0.95 (d, J=6.6 Hz, 1H).

### Example 39: 3-[(3R,5S)-5-Methyl-1-(8-trifluoromethyl-quinolin-5-yl)-piperidin-3-ylamino]-propane-1-sulfonic acid amide

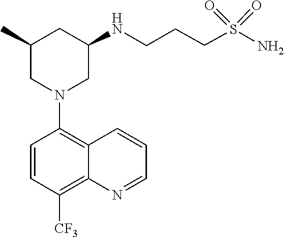 [SMILES: C[C@H]1C[C@@H](NCCCS(N)(=O)=O)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine and and 3-Bromo-propane-1-sulfonic acid amide. LC-MS (M+1)=431. ¹H NMR (400 MHz, DMSO-d₆) δ 9.01 (dd, J=4.1, 1.9 Hz, 1H), 8.56-8.45 (m, 1H), 8.05 (d, J=8.0 Hz, 1H), 7.66 (dd, J=8.8, 4.2 Hz, 1H), 7.19 (d, J=8.0 Hz, 1H), 6.74 (s, 2H), 3.53 (d, J=11.7 Hz, 1H), 3.02 (dd, J=9.2, 6.4 Hz, 2H), 2.91 (s, 1H), 2.69 (h, J=5.2 Hz, 2H), 2.37 (td, J=11.3, 4.0 Hz, 2H), 2.15-1.90 (m, 2H), 1.82 (q, J=7.3 Hz, 2H), 0.94 (d, J=6.4 Hz, 3H), 0.86 (q, J=11.9 Hz, 1H).

### Example 40: {2-[(3R,5S)-1-(8-Cyano-quinolin-5-yl)-5-trifluoromethyl-piperidin-3-ylamino]-ethyl}-urea

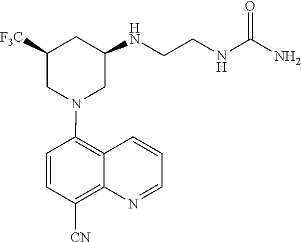 [SMILES: N#Cc1ccc(N2C[C@H](NCCNC(N)=O)C[C@H](C(F)(F)F)C2)c2cccnc12]

The title compound was prepared from 5-((3R,5S)-3-Amino-5-trifluoromethyl-piperidin-1-yl)-quinoline-8-carbonitrile and (2-Chloro-ethyl)-urea. LC-MS (M+1)=407. ¹H NMR (400 MHz, DMSO-d₆) δ 9.10-8.99 (m, 1H), 8.52 (d, J=8.5 Hz, 1H), 8.24 (d, J=8.0 Hz, 1H), 7.72 (dd, J=8.6, 4.2 Hz, 1H), 7.31 (d, J=8.1 Hz, 1H), 5.89 (d, J=5.9 Hz, 1H), 5.42 (s, 2H), 3.57 (t, J=12.6 Hz, 2H), 3.03 (q, J=6.4 Hz, 3H), 2.88 (t, J=11.4 Hz, 1H), 2.63 (d, J=6.5 Hz, 2H), 2.29 (d, J=12.5 Hz, 1H), 1.87 (s, 1H), 1.25 (q, J=12.0 Hz, 1H).

### Example 41: N-{2-[(3R,5S)-1-(8-Cyano-quinoxalin-5-yl)-5-methyl-piperidin-3-ylamino]-ethyl}-methanesulfonamide

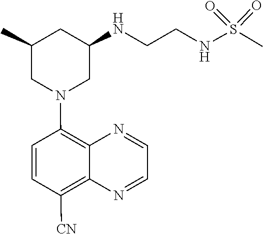

The title compound was prepared form 8-((3R,5S)-3-Amino-5-methyl-piperidin-1-yl)-quinoxaline-5-carbonitrile hydrochloride (2) and N-(2-Bromo-ethyl)-methanesulfonamide. LC-MS (M+1)=389. ¹H NMR (400 MHz, DMSO-d₆) δ 8.98 (dd, J=32.5, 1.7 Hz, 2H), 8.17 (d, J=8.4 Hz, 1H), 7.20 (d, J=8.5 Hz, 1H), 6.92 (s, 1H), 4.46 (d, J=12.3 Hz, 1H), 4.14 (d, J=12.2 Hz, 1H), 3.02 (t, J=6.5 Hz, 2H), 2.91 (s, 3H), 2.83-2.67 (m, 2H), 2.59 (dt, J=17.0, 11.3 Hz, 3H), 2.05 (d, J=12.4 Hz, 1H), 1.82 (d, J=44.5 Hz, 2H), 1.04-0.84 (m, 3H).

### Example 42: 8-{(3R,5S)-3-[(1,1-Dioxo-tetrahydro-1lambda6-thiophen-3-ylmethyl)-amino]-5-methyl-piperidin-1-yl}-quinoxaline-5-carbonitrile

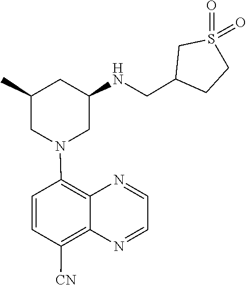 [SMILES: C[C@H]1C[C@@H](NCC2CCS(=O)(=O)C2)CN(c2ccc(C#N)c3nccnc23)C1]

The title compound was prepared from 8-((3R,5S)-3-Amino-5-methyl-piperidin-1-yl)-quinoxaline-5-carbonitrile hydrochloride (2) and 3-Bromomethyl-tetrahydro-thiophene 1,1-dioxide. LC-MS (M+1)=400. ¹H NMR (400 MHz, DMSO-d₆) δ 9.10-8.86 (m, 2H), 8.16 (d, J=8.4 Hz, 1H), 7.19 (d, J=8.5 Hz, 1H), 4.44 (d, J=11.7 Hz, 1H), 4.15 (d, J=12.4 Hz, 1H), 3.26-3.10 (m, 2H), 3.04 (q, J=12.2, 10.6 Hz, 1H), 2.86-2.65 (m, 3H), 2.65-2.54 (m, 2H), 2.22 (s, 1H), 2.05 (d, J=13.1 Hz, 1H), 1.94-1.70 (m, 3H), 0.93 (d, J=6.8 Hz, 4H).

### Example 43: 8-{(3R,5S)-3-[2-(1,1-Dioxo-1lambda6-thietan-3-yl)-ethylamino]-5-methyl-piperidin-1-yl}-quinoxaline-5-carbonitrile

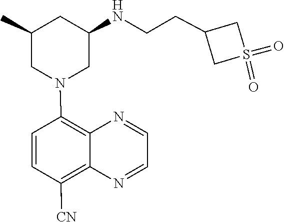 [SMILES: C[C@H]1C[C@@H](NCCC2CS(=O)(=O)C2)CN(c2ccc(C#N)c3nccnc23)C1]

The title compound was prepared from 8-((3R,5S)-3-Amino-5-methyl-piperidin-1-yl)-quinoxaline-5-carbonitrile hydrochloride (2) and 3-(2-Bromo-ethyl)-thietane 1,1-dioxide. LC-MS (M+1)=400. ¹H NMR (400 MHz, Methanol-d₄) δ 8.92 (d, J=17.1 Hz, 2H), 8.10 (d, J=8.4 Hz, 1H), 7.23 (d, J=8.4 Hz, 1H), 4.58 (d, J=11.9 Hz, 1H), 4.26 (dd, J=14.1, 9.6 Hz, 2H), 4.21-4.07 (m, 1H), 3.86 (dd, J=14.2, 6.6 Hz, 2H), 3.02 (t, J=10.9 Hz, 1H), 2.73 (q, J=6.6 Hz, 1H), 2.63 (dt, J=13.5, 6.6 Hz, 2H), 2.20 (d, J=12.5 Hz, 1H), 1.93 (q, J=7.4 Hz, 2H), 1.04 (d, J=6.8 Hz, 3H).

### Example 44: N-{2-[(3R,5S)-1-(8-Cyano-quinolin-5-yl)-5-trifluoromethyl-piperidin-3-ylamino]-ethyl}-acetamide

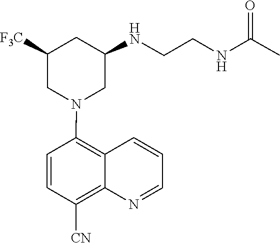 [SMILES: CC(=O)NCCN[C@@H]1C[C@H](C(F)(F)F)CN(c2ccc(C#N)c3ncccc23)C1]

The title compound was prepared from 5-((3R,5S)-3-Amino-5-trifluoromethyl-piperidin-1-yl)-quinoline-8-carbonitrile and N-(2-Chloro-ethyl)-acetamide. LC-MS (M+1)=406.

¹H NMR (400 MHz, DMSO-d₆) δ 9.05 (d, J=4.3 Hz, 1H), 8.70-8.52 (m, 1H), 8.22 (d, J=8.0 Hz, 1H), 7.70 (dd, J=8.5, 4.2 Hz, 1H), 7.32 (d, J=8.1 Hz, 1H), 6.37 (s, 1H), 4.02 (s, 1H), 3.69 (s, 1H), 3.56 (d, J=11.1 Hz, 1H), 3.44 (s, 2H), 3.13 (s, 3H), 2.93 (s, 1H), 2.68 (s, 1H), 2.01 (d, J=19.4 Hz, 1H), 1.96-1.66 (m, 3H), 1.60-1.30 (m, 1H).

### Example 45: {2-[(3R,5S)-5-Methyl-1-(8-trifluoromethyl-quinolin-5-yl)-piperidin-3-ylamino]-ethyl}-urea

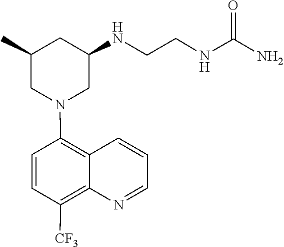 [SMILES: C[C@H]1C[C@@H](NCCNC(N)=O)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine and (2-Chloro-ethyl)-urea. LC-MS (M+1)=396. ¹H NMR (400 MHz, DMSO-d₆) δ 9.01 (d, J=4.2 Hz, 1H), 8.54-8.42 (m, 1H), 8.05 (d, J=8.1 Hz, 1H), 7.67 (dd, J=8.6, 4.2 Hz, 1H), 7.20 (d, J=8.1 Hz, 1H), 5.94 (d, J=34.6 Hz, 1H), 5.45 (d, J=22.7 Hz, 2H), 3.53 (d, J=11.1 Hz, 1H), 3.03 (q, J=6.1 Hz, 2H), 2.92 (s, 1H), 2.67-2.54 (m, 2H), 2.37 (q, J=10.2, 9.5 Hz, 2H), 2.18-1.89 (m, 2H), 1.65 (s, 1H), 0.94 (d, J=6.5 Hz, 3H), 0.86 (q, J=11.8 Hz, 1H).

### Example 46: Ethane sulfonic acid {2-[(3R,5S)-1-(8-cyano-quinoxalin-5-yl)-5-methyl-piperidin-3-ylamino]-ethyl}-amide

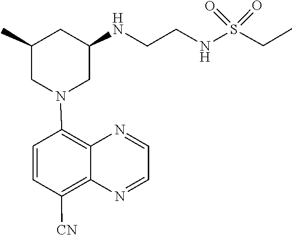 [SMILES: CCS(=O)(=O)NCCN[C@@H]1C[C@H](C)CN(c2ccc(C#N)c3nccnc23)C1]

The title compound was prepared from 8-((3R,5S)-3-Amino-5-methyl-piperidin-1-yl)-quinoxaline-5-carbonitrile and Ethanesulfonic acid (2-bromo-ethyl)-amide. LC-MS (M+1)=403. ¹H NMR (400 MHz, DMSO-d₆) δ 8.98 (d, J=33.6 Hz, 2H), 8.16 (d, J=8.3 Hz, 1H), 7.19 (d, J=8.6 Hz, 1H), 6.95 (s, 1H), 4.46 (d, J=11.8 Hz, 1H), 4.13 (d, J=12.3 Hz, 1H), 3.01 (q, J=7.4 Hz, 3H), 2.76 (d, J=36.2 Hz, 3H), 2.59 (dd, J=20.2, 10.9 Hz, 1H), 2.04 (d, J=12.7 Hz, 1H), 1.83 (d, J=41.6 Hz, 2H), 1.25-1.11 (m, 2H), 0.93 (t, J=7.5 Hz, 3H).

### Example 47: 8-{(3S,5R)-3-Methyl-5-[(oxetan-3-ylmethyl)-amino]-piperidin-1-yl}-quinoxaline-5-carbonitrile

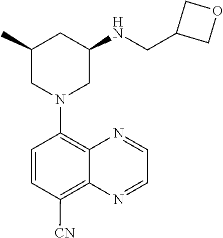 [SMILES: C[C@H]1C[C@@H](NCC2COC2)CN(c2ccc(C#N)c3nccnc23)C1]

The title compound was prepared from 8-((3R,5S)-3-Amino-5-methyl-piperidin-1-yl)-quinoxaline-5-carbonitrile and 3-Bromomethyl-oxetane. LC-MS (M+1)=338. ¹H NMR (400 MHz, DMSO-d₆) δ 8.98 (d, J=31.1 Hz, 2H), 8.16 (d, J=8.4 Hz, 1H), 7.19 (d, J=8.5 Hz, 1H), 4.62 (t, J=6.9 Hz, 2H), 4.44 (d, J=12.5 Hz, 1H), 4.27 (s, 2H), 4.16 (d, J=12.7 Hz, 1H), 3.0 (p, J=7.0 Hz, 1H), 2.88 (d, J=7.4 Hz, 2H), 2.77 (s, 1H), 2.58 (q, J=11.2, 10.8 Hz, 2H), 2.06 (d, J=12.6 Hz, 1H), 1.88 (s, 1H), 1.69 (s, 1H), 0.99-0.84 (m, 3H).

### Example 48: 5-{[(3R,5S)-5-Methyl-1-(8-trifluoromethyl-quinolin-5-yl)-piperidin-3-ylamino]-methyl}-pyrrolidin-2-one

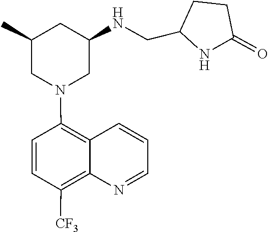 [SMILES: C[C@H]1C[C@@H](NCC2CCC(=O)N2)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride and 5-Bromomethyl-pyrrolidin-2-one. LC-MS (M+1)=407. ¹H NMR (400 MHz, DMSO-d₆) δ 9.01 (dd, J=4.1, 1.7 Hz, 1H), 8.48 (dd, J=8.6, 1.8 Hz, 1H), 8.05 (d, J=8.1 Hz, 1H), 7.67 (dd, J=8.6, 4.2 Hz, 1H), 7.60 (s, 1H), 7.20 (d, J=8.1 Hz, 1H), 3.63-3.46 (m, 3H), 2.93 (s, 1H), 2.58 (tt, J=11.8, 6.9 Hz, 2H), 2.38 (t, J=10.9 Hz, 2H), 2.19-1.89 (m, 4H), 1.81-1.56 (m, 2H), 0.94 (d, J=6.5 Hz, 3H), 0.84 (t, J=11.8 Hz, 1H).

### Example 49: N-{2-[(3R,5S)-1-(8-Cyano-quinoxalin-5-yl)-5-methyl-piperidin-3-ylamino]-ethyl}-acetamide

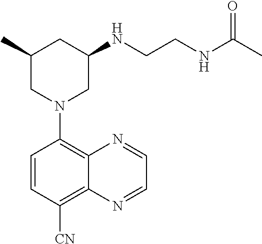 [SMILES: CC(=O)NCCN[C@@H]1C[C@H](C)CN(c2ccc(C#N)c3nccnc23)C1]

The title compound was prepared from 8-((3R,5S)-3-Amino-5-methyl-piperidin-1-yl)-quinoxaline-5-carbonitrile hydrochloride and N-(2-Chloro-ethyl)-acetamide. LC-MS (M+1)=353. ¹H NMR (400 MHz, Methanol-d₄) δ 8.92 (dd, J=15.3, 1.8 Hz, 2H), 8.10 (d, J=8.4 Hz, 1H), 7.22 (d, J=8.4 Hz, 1H), 4.66-4.52 (m, 1H), 4.21-4.04 (m, 1H), 3.36 (t, J=6.5 Hz, 2H), 3.10-2.98 (m, 1H), 2.85 (td, J=6.5, 2.4 Hz, 2H), 2.70-2.54 (m, 2H), 2.19 (d, J=12.8 Hz, 1H), 2.12-2.01 (m, 1H), 1.97 (s, 3H), 1.15 (t, J=7.3 Hz, 1H), 1.11-0.99 (m, 3H).

### Example 50: 4-{[(3R,5S)-5-Methyl-1-(8-trifluoromethyl-quinolin-5-yl)-piperidin-3-ylamino]-methyl}-tetrahydro-pyran-4-ol

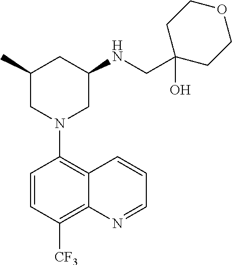 [SMILES: C[C@H]1C[C@@H](NCC2(O)CCOCC2)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride and 4-Bromomethyl-tetrahydro-pyran-4-ol. LC-MS (M+1)=424. ¹H NMR (400 MHz, DMSO-d₆) δ 9.01 (dd, J=4.1, 1.7 Hz, 1H), 8.53-8.44 (m, 1H), 8.06 (d, J=8.2 Hz, 1H), 7.66 (dd, J=8.6, 4.1 Hz, 1H), 7.20 (d, J=8.0 Hz, 1H), 4.19 (s, 1H), 3.59 (q, J=13.3, 11.9 Hz, 4H), 2.90 (s, 1H), 2.55 (s, 3H), 2.39 (t, J=11.6 Hz, 2H), 2.17-1.96 (m, 2H), 1.53 (dd, J=17.0, 7.6 Hz, 2H), 1.37 (d, J=13.4 Hz, 2H), 1.04-0.82 (m, 4H).

### Example 51: 1-(3-Hydroxy-azetidin-1-yl)-2-[(3R,5S)-5-methyl-1-(8-trifluoromethyl-quinolin-5-yl)-piperidin-3-ylamino]-ethanone

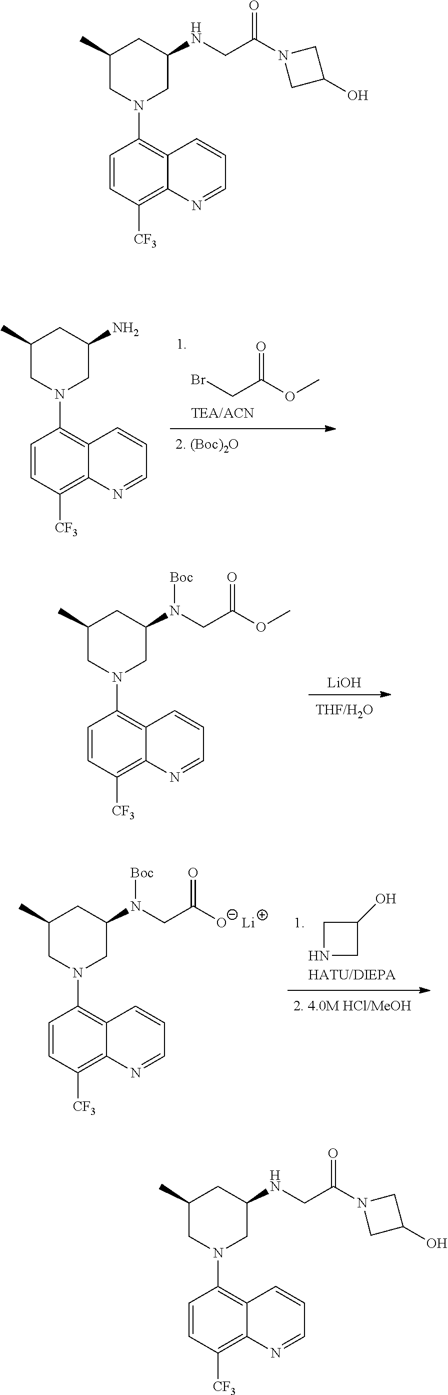

{tert-Butoxycarbonyl-[(3R,5S)-5-methyl-1-(8-trifluoromethyl-quinolin-5-yl)-piperidin-3-yl]-amino}-acetic acid methyl ester: A mixture of (3R,5S)-5-Methyl-1-(8-trifluoromethyl-quinolin-5-yl)-piperidin-3-ylamine hydrochloride (220 mg; 0.64 mmol; 1.0 eq.), Bromo-acetic acid methyl ester (146 mg; 0.95 mmol; 1.50 eq.), Triethyl-amine (0.27 ml; 1.91 mmol; 3.0 eq.) and ACN (3 ml) in a 10 ml microwave tube were stirred at 80° C. for 7 hr, until the reaction was completed. The reaction mixture was cooled to rt and then added tert-butoxycarbonyl tert-butyl carbonate (208 mg; 0.95 mmol; 1.50 eq.). The mixture was stirred at RT overnight, until the reaction was finished. The solvent was removed and the residue was loaded on 25 g silica column, eluting with hexane/EA 0-50%, to yield the title compound (128 mg, yield: 42%). LC-MS (M+1)=482.

{tert-Butoxycarbonyl-[(3R,5S)-5-methyl-1-(8-trifluoromethyl-quinolin-5-yl)-piperidin-3-yl]-amino}-acetic acid lithium: A mixture of {tert-Butoxycarbonyl-[(3R,5S)-5-methyl-1-(8-trifluoromethyl-quinolin-5-yl)-piperidin-3-yl]-amino}-acetic acid methyl ester (128 mg; 0.27 mmol; 1.0 eq.), lithium hydroxide hydrate (22 mg; 0.53 mmol; 2.0 eq.) in THF (2 ml) and water (2 ml) was stirred at RT overnight. The solvents were removed to yield a yellow solid as the title compound. LC-MS (M+1)=467.

1-(3-Hydroxy-azetidin-1-yl)-2-[(3R,5S)-5-methyl-1-(8-trifluoromethyl-quinolin-5-yl)-piperidin-3-ylamino]-ethanone: To a solution of {tert-Butoxycarbonyl-[(3R,5S)-5-methyl-1-(8-trifluoromethyl-quinolin-5-yl)-piperidin-3-yl]-amino}-acetic acid lithium (50 mg; 0.11 mmol; 1.0 eq.) in DMF (1 ml) was added HATU (60 mg; 0.16 mmol; 1.50 eq.). The resulting mixture was stirred at RT for 20 min, and then added Ethyl-diisopropyl-amine (0.03 ml; 0.16 mmol; 1.50 eq.) and azetidin-3-ol (0.02 ml; 0.21 mmol; 2.0 eq.). The mixture was stirred for additional 1 hr at RT, until the reaction was completed. The reaction was diluted with water (30 ml), extracted with EA (30 ml×2). The combined organic layers were washed with 10% citric acid, brine, 5% NaHCO₃, then brine, dried over Na₂SO₄ and concentrated. The residue was dissolved in 1 ml methanol and added hydrogen chloride (4.0M in dioxane) (0.18 ml; 0.74 mmol; 7.0 eq.), The mixture was stirred at RT for 2 hr until the reaction was completed. The solvents were removed and the residue was purified by prep HPLC, eluting with 0-60% CAN/water (contained 0.1% ammonia) to provide the title compound (18 mg, yield: 40%). LC-MS (M+1)=423. ¹H NMR (400 MHz, Methanol-d₄) δ 9.03-8.83 (m, 1H), 8.58 (d, J=8.7 Hz, 1H), 8.05 (d, J=8.1 Hz, 1H), 7.63 (d, J=5.0 Hz, 1H), 7.25 (d, J=8.1 Hz, 1H), 4.60 (d, J=6.3 Hz, 1H), 4.40 (s, 1H), 4.24 (s, 1H), 4.02-3.88 (m, 1H), 3.80 (s, 1H), 3.61 (d, J=11.5 Hz, 1H), 3.33 (s, 1H), 3.07 (d, J=11.4 Hz, 1H), 2.48 (dt, J=31.4, 11.2 Hz, 2H), 2.29-1.96 (m, 2H), 1.12-0.85 (m, 4H).

The following compound was synthesized in an analogous manner.

### Example 52: N-Methoxy-4-[(3R,5S)-5-methyl-1-(8-trifluoromethyl-quinoxalin-5-yl)-piperidin-3-ylamino]-butyramide

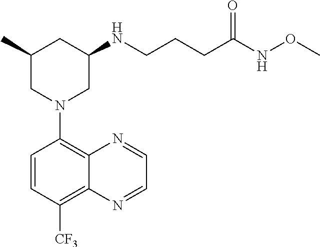

The title compound was prepared from 4-{tert-Butoxycarbonyl-[(3R,5S)-5-methyl-1-(8-trifluoromethyl-quinoxalin-5-yl)-piperidin-3-yl]-amino}-butyric acid lithium and O-Methyl-hydroxylamine hydrochloride. LC-MS (M+1)=426. ¹H NMR (400 MHz, Methanol-d₄) δ 8.92 (d, J=4.4 Hz, 2H), 8.04 (d, J=8.1 Hz, 1H), 7.24 (d, J=8.4 Hz, 1H), 5.50-5.31 (m, 1H), 4.40 (d, J=11.8 Hz, 1H), 4.06-3.91 (m, 1H), 3.70 (s, 3H), 3.09 (s, 2H), 2.86-2.67 (m, 2H), 2.54 (t, J=11.3 Hz, 2H), 2.17 (d, J=8.2 Hz, 2H), 2.07 (s, 1H), 1.87 (s, 2H), 1.17 (s, 1H), 1.04 (d, J=6.2 Hz, 3H).

### Example 53: 1-(3-Hydroxy-azetidin-1-yl)-3-[(3R,5S)-5-methyl-1-(8-trifluoromethyl-quinolin-5-yl)-piperidin-3-ylamino]-propan-1-one

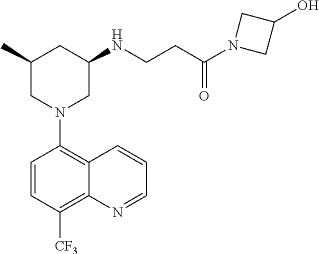 [SMILES: C[C@H]1C[C@@H](NCCC(=O)N2CC(O)C2)CN(c2ccc(C(F)(F)F)c3ncccc23)C1]

The title compound was prepared from 3-{tert-Butoxycarbonyl-[(3R,5S)-5-methyl-1-(8-trifluoromethyl-quinolin-5-yl)-piperidin-3-yl]-amino}-propionic acid lithium and Azetidin-3-ol. LC-MS (M+1)=437. ¹H NMR (400 MHz, DMSO-d₆) δ 9.01 (dd, J=4.1, 1.9 Hz, 1H), 8.49 (dd, J=8.7, 2.1 Hz, 1H), 8.05 (d, J=8.1 Hz, 1H), 7.66 (dd, J=8.7, 3.9 Hz, 1H), 7.20 (d, J=8.1 Hz, 1H), 5.68 (d, J=6.2 Hz, 1H), 4.43 (d, J=6.3 Hz, 1H), 4.27 (t, J=7.9 Hz, 1H), 4.0 (t, J=8.6 Hz, 1H), 3.82 (d, J=8.3 Hz, 1H), 3.55 (d, J=5.0 Hz, 2H), 3.18 (s, 1H), 2.96 (s, 1H), 2.80 (s, 2H), 2.38 (d, J=9.3 Hz, 2H), 2.18 (d, J=8.0 Hz, 2H), 2.13-1.83 (m, 2H), 0.94 (d, J=6.1 Hz, 3H), 0.86 (d, J=12.0 Hz, 1H).

### Example 54: N-(1,1-Dioxo-1lambda6-thietan-3-yl)-3-[(3R,5S)-5-methyl-1-(8-trifluoromethyl-quinolin-5-yl)-piperidin-3-ylamino]-propionamide

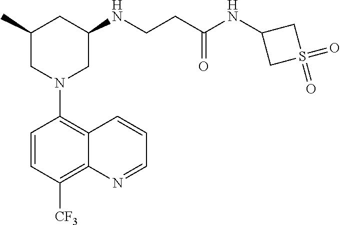 [SMILES: C[C@H]1C[C@@H](NCCC(=O)NC2CS(=O)(=O)C2)CN(c2ccc(C(F)(F)F)c3ncccc23)C1]

The title compound was prepared from 3-{tert-Butoxycarbonyl-[(3R,5S)-5-methyl-1-(8-trifluoromethyl-quinolin-5-yl)-piperidin-3-yl]-amino}-propionic acid lithium and 1,1-Dioxo-1lambda6-thietan-3-ylamine. LC-MS (M+1)=485. ¹H NMR (400 MHz, DMSO-d₆) δ 9.11-8.96 (m, 1H), 8.77 (s, 1H), 8.50 (d, J=8.9 Hz, 1H), 8.22-8.0 (m, 2H), 7.68 (s, 1H), 7.22 (d, J=8.6 Hz, 1H), 4.52 (t, J=11.3 Hz, 2H), 4.34 (d, J=7.9 Hz, 1H), 4.03 (d, J=11.8 Hz, 3H), 3.59 (d, J=11.4 Hz, 2H), 3.13 (s, 1H), 2.95 (s, 2H), 2.35 (s, 2H), 2.10 (dd, J=37.0, 14.6 Hz, 2H), 0.96 (d, J=6.9 Hz, 3H).

### Example 55: N-Methoxy-3-[(3R,5S)-5-methyl-1-(8-trifluoromethyl-quinolin-5-yl)-piperidin-3-ylamino]-propionamide

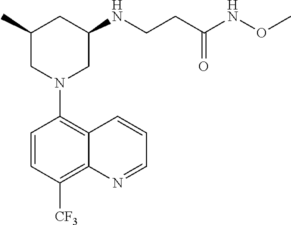

The title compound was prepared from 3-{tert-Butoxycarbonyl-[(3R,5S)-5-methyl-1-(8-trifluoromethyl-quinolin-5-yl)-piperidin-3-yl]-amino}-propionic acid lithium and O-Methyl-hydroxylamine hydrochloride. LC-MS (M+1)=411. ¹H NMR (400 MHz, DMSO-d₆) δ 9.04 (d, J=4.1 Hz, 1H), 8.53 (t, J=7.2 Hz, 1H), 8.16-8.03 (m, 1H), 7.69 (dd, J=8.8, 4.0 Hz, 1H), 7.26 (d, J=8.0 Hz, 1H), 3.60 (s, 4H), 3.13 (s, 1H), 2.82-2.57 (m, 2H), 2.40-2.15 (m, 2H), 2.06 (s, 1H), 1.22-1.02 (m, 1H), 0.98 (d, J=6.5 Hz, 2H).

### Example 56: N-Methyl-3-[(3R,5S)-5-methyl-1-(8-trifluoromethyl-quinolin-5-yl)-piperidin-3-ylamino]-propionamide

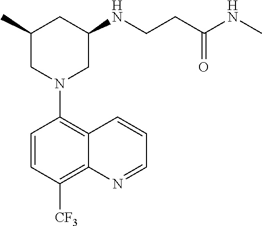

The title compound was prepared from 3-{tert-Butoxycarbonyl-[(3R,5S)-5-methyl-1-(8-trifluoromethyl-quinolin-5-yl)-piperidin-3-yl]-amino}-propionic acid lithium and methanamine hydrochloride. LC-MS (M+1)=395. ¹H NMR (400 MHz, Methanol-d₄) δ 8.97 (s, 1H), 8.61 (d, J=8.2 Hz, 1H), 8.07 (d, J=7.5 Hz, 1H), 7.64 (s, 1H), 7.30 (d, J=7.9 Hz, 1H), 3.72 (s, 1H), 3.56-3.37 (m, 2H), 3.23 (s, 2H), 2.76 (s, 3H), 2.63-2.42 (m, 2H), 2.35 (d, J=12.5 Hz, 1H), 2.20 (s, 1H), 1.17 (d, J=12.2 Hz, 1H), 1.14-0.93 (m, 3H).

### Example 57: 5-((3R,5S)-3-Amino-5-methyl-piperidin-1-yl)-7-fluoro-quinoline-8-carbonitrile

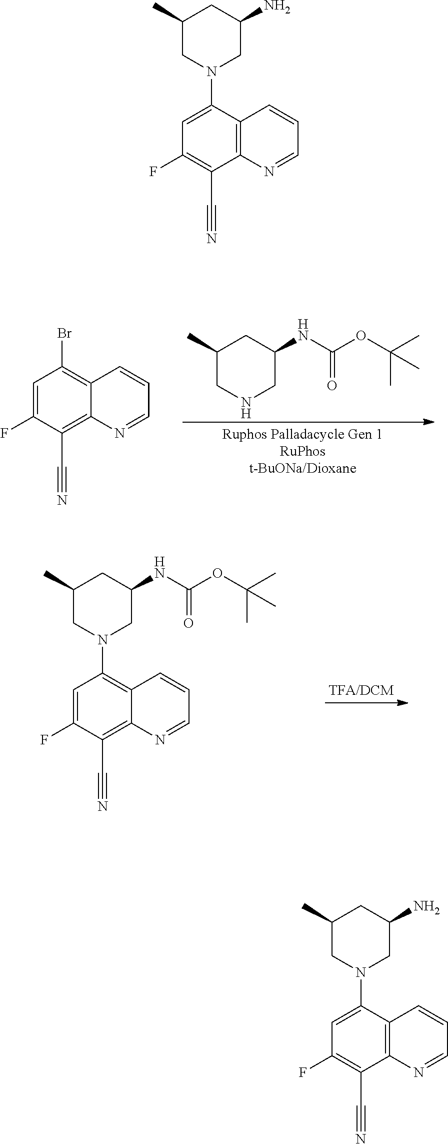 [SMILES: C[C@H]1C[C@@H](N)CN(c2cc(F)c(C#N)c3ncccc23)C1 ; N#Cc1c(F)cc(Br)c2cccnc12 ; C[C@@H]1CNC[C@H](NC(=O)OC(C)(C)C)C1 ; C[C@H]1C[C@@H](NC(=O)OC(C)(C)C)CN(c2cc(F)c(C#N)c3ncccc23)C1 ; C[C@H]1C[C@@H](N)CN(c2cc(F)c(C#N)c3ncccc23)C1]

[(3R,5S)-1-(8-Cyano-7-fluoro-quinolin-5-yl)-5-methyl-piperidin-3-yl]-carbamic acid tert-butyl ester: A mixture of 5-Bromo-7-fluoro-quinoline-8-carbonitrile (100 mg; 0.40 mmol; 1.0 eq.), ((3R,5S)-5-Methyl-piperidin-3-yl)-carbamic acid tert-butyl ester (85 mg; 0.40 mmol; 1.0 eq.), chloro(2-dicyclohexylphosphino-2′,6′-di-i-propoxy-1,1′-biphenyl)[2-(2-aminoethylphenyl)]palladium(ii), methyl-t-butyl ether adduct (16 mg; 0.02 mmol; 0.05 eq.), 2-dicyclohexylphosphino-2′,6′-di-i-propoxy-1,1′-biphenyl (9 mg, 0.02 mmol, 0.05 eq.), sodium tert-butoxide (42 mg, 0.44 mmol, 1.1 eq) and dioxane (2 ml) in 5 ml microwave tube was degased, and then microwaved at 100° C. for 60 mins. LCMS indicated that the reaction was completed. The reaction mixture was concentrated under reduced pressure. The residue was purified by chromatography on a silica column 50 g, eluting with EA/hexane 20-80%, to yield the title compound, which was directly used for the next step reaction. LC-MS (M+1)=385.

5-((3R,5S)-3-Amino-5-methyl-piperidin-1-yl)-7-fluoro-quinoline-8-carbonitrile: To [(3R,5S)-1-(8-Cyano-7-fluoro-quinolin-5-yl)-5-methyl-piperidin-3-yl]-carbamic acid tert-butyl ester (110 mg; 0.29 mmol; 1.0 eq) in DCM (0.6 ml) was added Trifluoro-acetic acid (652 mg; 5.72 mmol; 20.0 eq.). The mixture was stirred at RT for 10 min, until the reaction was completed. The solvent was removed and the residue was purified by prep waters, eluting with 10-50% ACN/water (contained 0.1% ammonia) to give the title compound. LC-MS (M+1)=285. ¹H NMR (400 MHz, DMSO-d₆) δ 9.05 (ddd, J=15.7, 4.2, 1.6 Hz, 1H), 8.43 (dd, J=8.6, 1.7 Hz, 1H), 7.63 (dd, J=8.6, 4.3 Hz, 1H), 7.15 (d, J=12.4 Hz, 1H), 3.57 (d, J=13.0 Hz, 1H), 3.44 (d, J=12.1 Hz, 1H), 3.0 (td, J=10.7, 5.4 Hz, 2H), 2.50-2.54 (m, 1H), 2.05-1.86 (m, 2H), 1.60 (s, 2H), 0.93 (d, J=6.4 Hz, 3H), 0.85 (d, J=12.3 Hz, 1H).

### Example 58: N-[(3R,5S)-1-(8-Cyano-7-fluoro-quinolin-5-yl)-5-methyl-piperidin-3-yl]-2-(1-methyl-1H-pyrazol-4-yl)-acetamide

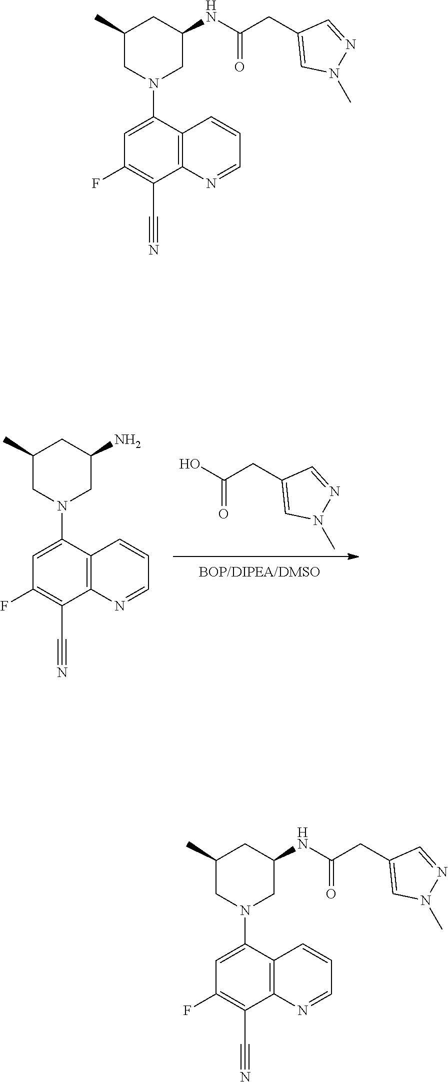

To a solution of 5-((3R,5S)-3-Amino-5-methyl-piperidin-1-yl)-7-fluoro-quinoline-8-carbonitrile (40. mg; 0.14 mmol; 1.0 eq.), (1-Methyl-1H-pyrazol-4-yl)-acetic acid (29 mg; 0.21 mmol; 1.50 eq.) and DIEPA (0.05 ml; 0.28 mmol; 2.0 eq.) in DMSO (2 ml) was added benzotriazol-1-yloxy-tris(dimethylamino)phosphonium hexafluorophosphate (93 mg; 0.21 mmol; 1.50 eq.). The resulting mixture was stirred at RT for 1 hr, until the reaction was completed. The crude was purified by prep HPLC, eluting with 20-60% ACN/water (contained 0.1% ammonia), to yield the title compound. LC-MS (M+1)=407. ¹H NMR (400 MHz, Methanol-d₄) δ 9.04-8.91 (m, 1H), 8.59 (dd, J=8.6, 1.6 Hz, 1H), 7.61 (dd, J=8.6, 4.3 Hz, 1H), 7.50 (s, 1H), 7.38 (s, 1H), 7.10 (d, J=11.8 Hz, 1H), 4.19 (t, J=11.3 Hz, 1H), 3.85 (s, 3H), 3.79 (d, J=11.8 Hz, 1H), 3.52 (d, J=12.1 Hz, 1H), 3.38 (s, 2H), 2.58 (q, J=11.0 Hz, 2H), 2.13 (d, J=11.6 Hz, 1H), 1.22 (q, J=12.5 Hz, 2H), 1.05 (d, J=6.3 Hz, 3H).

### Example 59: N-[(3R,5S)-1-(8-Cyano-7-fluoro-quinolin-5-yl)-5-methyl-piperidin-3-yl]-2-(1-methyl-azetidin-3-yl)-acetamide

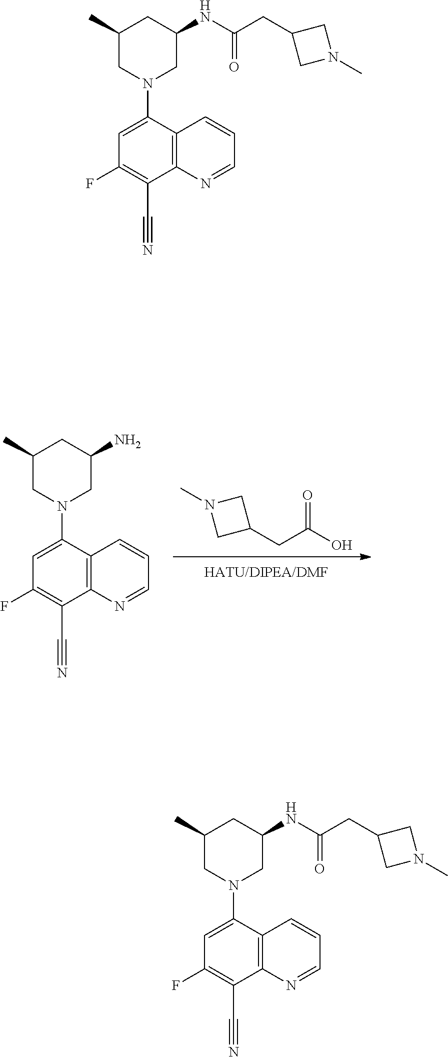

To a solution of 5-((3R,5S)-3-Amino-5-methyl-piperidin-1-yl)-7-fluoro-quinoline-8-carbonitrile (20 mg; 0.07 mmol; 1.0 eq.) in DMF (1 ml) was added HATU (45 mg; 0.12 mmol; 1.70 eq.). After stirring for 10 mins at RT, Ethyl-isopropyl-amine (0.04 ml; 0.21 mmol; 3.0 eq.) and 5-((3R,5S)-3-Amino-5-methyl-piperidin-1-yl)-7-fluoro-quinoline-8-carbonitrile (20 mg; 0.07 mmol; 1.0 eq.) were added. The resulting mixture was stirred at RT for 1 hr, until the reaction finished. The solvent was removed and the residue was purified by prep HPLC, eluting with 20-60% ACN/water (contained 0.1% ammonia) to yield the title compound. LC-MS (M+1)=396. ¹H NMR (400 MHz, Methanol-d₄) δ 8.97 (dd, J=4.3, 1.6 Hz, 1H), 8.59 (dd, J=8.6, 1.7 Hz, 1H), 7.62 (dd, J=8.6, 4.3 Hz, 1H), 7.11 (d, J=11.8 Hz, 1H), 4.24-4.11 (m, 1H), 3.78 (d, J=12.3 Hz, 1H), 3.51 (q, J=7.4 Hz, 2H), 3.05-2.92 (m, 3H), 2.80 (p, J=7.5 Hz, 1H), 2.61-2.50 (m, 2H), 2.47 (dd, J=7.7, 2.1 Hz, 2H), 2.32 (d, J=3.9 Hz, 3H), 2.18-2.02 (m, 2H), 1.22 (t, J=12.6 Hz, 1H), 1.05 (d, J=6.4 Hz, 3H).

The following compound was synthesized in an analogous manner.

### Example 60: N-[(3R,5S)-1-(8-Cyano-quinolin-5-yl)-5-trifluoromethyl-piperidin-3-yl]-2,3-dihydroxy-propionamide

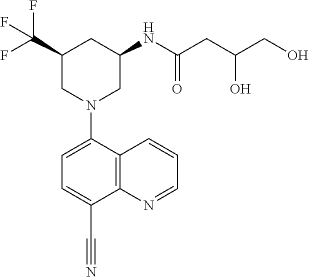 [SMILES: N#Cc1ccc(N2C[C@H](NC(=O)CC(O)CO)C[C@H](C(F)(F)F)C2)c2cccnc12]

The title compound was prepared from 5-((3R,5S)-3-Amino-5-trifluoromethyl-piperidin-1-yl)-quinoline-8-carbonitrile trifluoroacetate and 2,3-Dihydroxy-propionic acid. MS: 409 [M+H]⁺. 1H NMR (400 MHz, Methanol-d₄) δ 8.99 (s, 1H), 8.65 (d, J=7.9 Hz, 1H), 8.14 (d, J=8.1 Hz, 1H), 7.68 (dd, J=8.1, 3.9 Hz, 1H), 7.30 (dd, J=8.2, 2.5 Hz, 1H), 4.44-4.29 (m, 1H), 4.09 (p, J=4.3 Hz, 1H), 3.82-3.61 (m, 4H), 3.0 (q, J=13.7, 11.3 Hz, 2H), 2.72 (q, J=10.9 Hz, 1H), 2.36 (d, J=12.5 Hz, 1H), 1.77-1.62 (m, 1H).

### Example 61: 1-Methyl-piperidine-4-carboxylic acid [(3R,5S)-1-(8-cyano-quinolin-5-yl)-5-trifluoromethyl-piperidin-3-yl]-amide

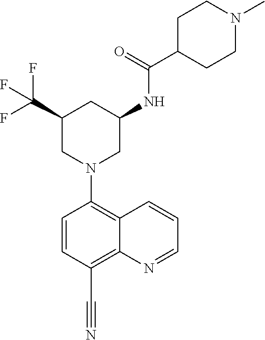 [SMILES: CN1CCC(C(=O)N[C@@H]2C[C@H](C(F)(F)F)CN(c3ccc(C#N)c4ncccc34)C2)CC1]

The title compound was prepared from 5-((3R,5S)-3-Amino-5-trifluoromethyl-piperidin-1-yl)-quinoline-8-carbonitrile trifluoroacetate and 1-Methyl-piperidine-4-carboxylic acid. MS: 446 [M+H]⁺. ¹H NMR (400 MHz, Methanol-d₄) δ 9.0 (dd, J=4.3, 1.5 Hz, 1H), 8.66 (dd, J=8.7, 1.6 Hz, 1H), 8.15 (d, J=8.0 Hz, 1H), 7.70 (dd, J=8.6, 4.2 Hz, 1H), 7.31 (d, J=8.0 Hz, 1H), 4.39-4.23 (m, 1H), 3.75-3.60 (m, 2H), 3.14-2.89 (m, 4H), 2.60 (t, J=11.2 Hz, 1H), 2.35 (d, J=12.6 Hz, 1H), 2.28 (s, 3H), 2.24-2.15 (m, 1H), 2.06 (ddd, J=14.9, 11.6, 7.0 Hz, 2H), 1.93-1.68 (m, 4H), 1.60 (q, J=12.1 Hz, 1H).

### Example 62: N-[(3R,5S)-1-(8-Cyano-quinolin-5-yl)-5-trifluoromethyl-piperidin-3-yl]-2-hydroxy-acetamide

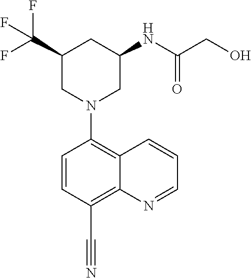 [SMILES: N#Cc1ccc(N2C[C@H](NC(=O)CO)C[C@H](C(F)(F)F)C2)c2cccnc12]

The title compound was prepared from 5-((3R,5S)-3-Amino-5-trifluoromethyl-piperidin-1-yl)-quinoline-8-carbonitrile trifluoroacetate and glycolic acid. MS: 379 [M+H]⁺. ¹H NMR (400 MHz, Methanol-d₄) δ 8.99 (dd, J=4.2, 1.4 Hz, 1H), 8.73-8.57 (m, 1H), 8.14 (d, J=8.0 Hz, 1H), 7.68 (dd, J=8.6, 4.2 Hz, 1H), 7.31 (d, J=8.0 Hz, 1H), 4.46-4.32 (m, 1H), 4.02 (s, 2H), 3.66 (d, J=8.5 Hz, 2H), 3.14-2.92 (m, 2H), 2.74 (t, J=11.3 Hz, 1H), 2.35 (d, J=12.2 Hz, 1H), 1.73 (q, J=12.2 Hz, 1H), 1.41-1.27 (m, 1H).

### Example 63: 2-(4-hydroxy-1-methylpiperidin-4-yl)-N-[(3R,5S)-5-(trifluoromethyl)-1-[8-(trifluoromethyl)quinoxalin-5-yl]piperidin-3-yl]acetamide

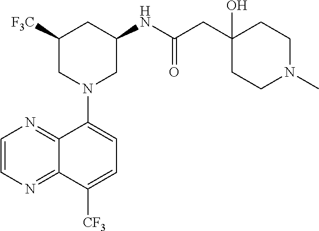 [SMILES: CN1CCC(O)(CC(=O)N[C@@H]2C[C@H](C(F)(F)F)CN(c3ccc(C(F)(F)F)c4nccnc34)C2)CC1]

The title compound was prepared from cis-5-(trifluoromethyl)-1-[8-(trifluoromethyl)quinoxalin-5-yl]piperidin-3-amine hydrogen chloride and 2-(4-hydroxy-1-methylpiperidin-4-yl)acetic acid. MS: 520 [M+H]⁺. ¹H NMR (400 MHz, Methanol-d₄, ppm) δ 8.98-8.91 (m, 2H), 8.08 (d, J=8.3 Hz, 1H), 7.35 (d, J=8.3 Hz, 1H), 4.50-4.44 (m, 1H), 4.33-4.21 (m, 1H), 4.20-4.11 (m, 1H), 3.0-2.90 (m, 2H), 2.80 (t, J=11.2 Hz, 1H), 2.68-2.60 (m, 2H), 2.54-2.42 (m, 2H), 2.39 (s,

### Example 64: 3-(dimethylamino)-N-[(3R,5S)-5-(trifluoromethyl)-1-[8-(trifluoromethyl)quinolin-5-yl]piperidin-3-yl]propanamide

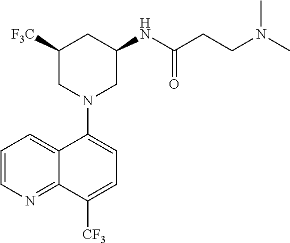 [SMILES: CN(C)CCC(=O)N[C@@H]1C[C@H](C(F)(F)F)CN(c2ccc(C(F)(F)F)c3ncccc23)C1]

The title compound was prepared from (3R,5S)-5-(trifluoromethyl)-1-[8-(trifluoromethyl)quinolin-5-yl]piperidin-3-amine and 3-(dimethylamino)propanoic acid. MS: 463 [M+H]⁺. ¹H NMR (400 MHz, Methanol-d₄, ppm) δ 8.97 (dd, J=4.2, 1.7 Hz, 1H), 8.69 (dd, J=8.6, 1.8 Hz, 1H), 8.08 (d, J=8.0 Hz, 1H), 7.67 (dd, J=8.6, 4.2 Hz, 1H), 7.32 (d, J=8.1 Hz, 1H), 4.39-4.26 (m, 1H), 3.71-3.58 (m, 2H), 3.12-2.90 (m, 2H), 2.71-2.62 (m, 2H), 2.58 (t, J=11.1 Hz, 1H), 2.49-2.33 (m, 3H), 2.28 (s, 6H), 1.63-1.50 (m, 1H).

### Example 65: N-[(3R,5S)-1-(8-cyanoquinolin-5-yl)-5-(trifluoromethyl)piperidin-3-yl]-3-(dimethylamino)propanamide

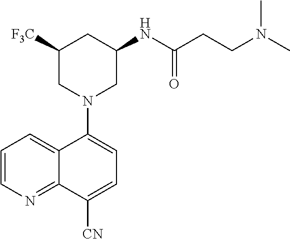 [SMILES: CN(C)CCC(=O)N[C@@H]1C[C@H](C(F)(F)F)CN(c2ccc(C#N)c3ncccc23)C1]

The title compound was prepared from 5-((3R,5S)-3-Amino-5-trifluoromethyl-piperidin-1-yl)-quinoline-8-carbonitrile and 3-(dimethylamino)propanoic acid. MS: 420 [M+H]⁺.

¹H NMR (400 MHz, Methanol-d₄, ppm) δ 9.01 (dd, J=4.2, 1.6 Hz, 1H), 8.68 (dd, J=8.6, 1.7 Hz, 1H), 8.17 (d, J=8.0 Hz, 1H), 7.70 (dd, J=8.6, 4.2 Hz, 1H), 7.33 (d, J=8.0 Hz, 1H), 4.38-4.26 (m, 1H), 3.80-3.64 (m, 2H), 3.14-2.96 (m, 2H), 2.74-2.66 (m, 2H), 2.61 (t, J=11.2 Hz, 1H), 2.48-2.35 (m, 3H), 2.31 (s, 6H), 1.65-1.52 (m, 1H).

### Example 66: 2-(4-methylpiperazin-1-yl)-N-[(3R,5S)-5-(trifluoromethyl)-1-[8-(trifluoromethyl)quinolin-5-yl]piperidin-3-yl]acetamide

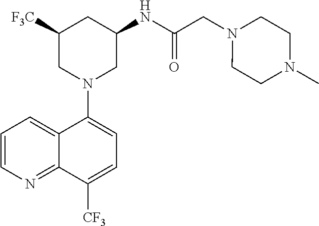 [SMILES: CN1CCN(CC(=O)N[C@@H]2C[C@H](C(F)(F)F)CN(c3ccc(C(F)(F)F)c4ncccc34)C2)CC1]

The title compound was prepared from (3R,5S)-5-(trifluoromethyl)-1-[8-(trifluoromethyl)quinolin-5-yl]piperidin-3-amine and 2-(4-methylpiperazin-1-yl)acetic acid. MS: 504 [M+H]⁺. ¹H NMR (400 MHz, Methanol-d₄, ppm) δ 9.0-8.94 (m, 1H), 8.69 (dd, J=8.6, 1.8 Hz, 1H), 8.08 (d, J=8.0 Hz, 1H), 7.66 (dd, J=8.6, 4.2 Hz, 1H), 7.32 (d, J=8.0 Hz, 1H), 4.40-4.35 (m, 1H), 3.65-3.57 (m, 2H), 3.09-3.06 (m, 3H), 3.02-2.91 (m, 1H), 2.75-2.39 (m, 9H), 2.39-2.32 (m, 1H), 2.32 (s, 3H), 1.75-1.61 (m, 1H).

### Example 67: 2-(4-hydroxy-1-methylpiperidin-4-yl)-N-[(3R,5S)-5-(trifluoromethyl)-1-[8-(trifluoromethyl)quinolin-5-yl]piperidin-3-yl]acetamide

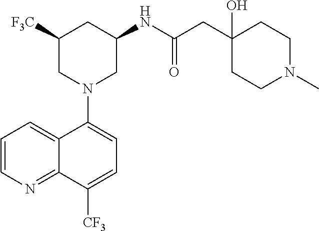 [SMILES: CN1CCC(O)(CC(=O)N[C@@H]2C[C@H](C(F)(F)F)CN(c3ccc(C(F)(F)F)c4ncccc34)C2)CC1]

The title compound was prepared from (3R,5S)-5-(trifluoromethyl)-1-[8-(trifluoromethyl)quinolin-5-yl]piperidin-3-amine and 2-(4-hydroxy-1-methylpiperidin-4-yl)acetic acid. MS: 519 [M+H]⁺. ¹H NMR (400 MHz, Methanol-d₄, ppm) δ 8.97 (dd, J=4.2, 1.7 Hz, 1H), 8.68 (dd, J=8.7, 1.8 Hz, 1H), 8.07 (d, J=8.0 Hz, 1H), 7.66 (dd, J=8.6, 4.2 Hz, 1H), 7.31 (d, J=8.0 Hz, 1H), 4.40-4.28 (m, 1H), 3.73-3.55 (m, 2H), 3.14-2.88 (m, 2H), 2.64-2.54 (m, 3H), 2.52-2.38 (m, 3H), 2.36 (s, 2H), 2.29 (s, 3H), 1.78-1.64 (m, 4H), 1.63-1.56 (m, 1H).

### Example 68: (3R,5S)-1-(7-fluoro-8-methylquinolin-5-yl)-5-(trifluoromethyl)piperidin-3-amine hydrochloride

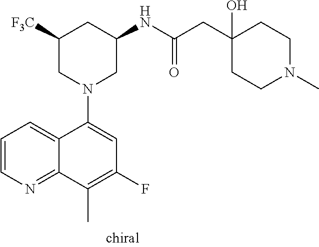 [SMILES: Cc1c(F)cc(N2C[C@H](NC(=O)CC3(O)CCN(C)CC3)C[C@H](C(F)(F)F)C2)c2cccnc12]

The title compound was prepared from (3R,5S)-1-(7-fluoro-8-methylquinolin-5-yl)-5-(trifluoromethyl)piperidin-3-amine and 2-(4-hydroxy-1-methylpiperidin-4-yl)acetic acid. MS: 483 [M+H]⁺. ¹H NMR (400 MHz, Methanol-d₄, ppm) δ 9.43-9.37 (m, 1H), 9.20-9.15 (m, 1H), 8.14-8.07 (m, 1H), 7.51 (d, J=11.1 Hz, 1H), 4.40-4.28 (m, 1H), 3.68-3.55 (m, 2H), 3.36-3.32 (m, 2H), 3.32-3.27 (m, 2H), 3.15-2.96 (m, 2H), 2.86 (s, 3H), 2.76-2.66 (m, 4H), 2.46 (s, 2H), 2.43-2.33 (m, 1H), 2.07-1.88 (m, 4H), 1.71-1.57 (m, 1H).

### Example 69: N-[(3R,5S)-1-(7-fluoro-8-methylquinolin-5-yl)-5-methylpiperidin-3-yl]-2-(4-hydroxy-1-methylpiperidin-4-yl)acetamide

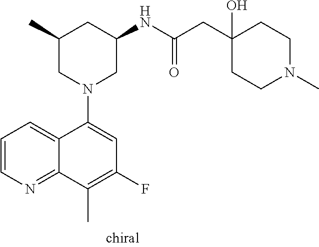 [SMILES: Cc1c(F)cc(N2C[C@@H](C)C[C@@H](NC(=O)CC3(O)CCN(C)CC3)C2)c2cccnc12]

The title compound was prepared from (3R,5S)-1-(7-fluoro-8-methylquinolin-5-yl)-5-methylpiperidin-3-amine and 2-(4-hydroxy-1-methylpiperidin-4-yl)acetic acid. MS: 429 [M+H]⁺. ¹H NMR (400 MHz, Methanol-d₄, ppm) δ 9.36 (dd, J=8.5, 1.5 Hz, 1H), 9.13 (dd, J=5.5, 1.6 Hz, 1H), 8.07 (dd, J=8.5, 5.5 Hz, 1H), 7.38 (d, J=11.5 Hz, 1H), 4.27-4.23 (m, 1H), 3.69-3.62 (m, 1H), 3.43-3.35 (m, 3H), 3.32-3.24 (m, 2H), 2.87 (s, 3H), 2.65 (s, 3H), 2.63-2.53 (m, 2H), 2.45 (s, 2H), 2.19-2.11 (m, 2H), 2.04-1.87 (m, 4H), 1.33-1.15 (m, 1H), 1.06 (d, J=6.4 Hz, 3H).

### Example 70: N-[(3R,5S)-1-(8-methyl-1,7-naphthyridin-5-yl)-5-(trifluoromethyl)piperidin-3-yl]-2-(4-methylpiperazin-1-yl)acetamide

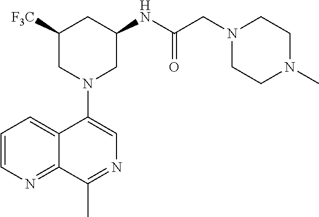 [SMILES: Cc1ncc(N2C[C@H](NC(=O)CN3CCN(C)CC3)C[C@H](C(F)(F)F)C2)c2cccnc12]

The title compound was prepared from (3R,5S)-1-(8-methyl-1,7-naphthyridin-5-yl)-5-(trifluoromethyl)piperidin-3-amine and 2-(4-methylpiperazin-1-yl)acetic acid. MS: 451 [M+H]⁺. ¹H NMR (300 MHz, Chloroform-d, ppm) δ 9.01 (dd, J=4.2, 1.7 Hz, 1H), 8.42 (dd, J=8.5, 1.8 Hz, 1H), 8.16 (s, 1H), 7.65 (dd, J=8.5, 4.1 Hz, 1H), 7.19 (d, J=8.6 Hz, 1H), 4.42-4.31 (m, 1H), 3.62-3.46 (m, 2H), 3.19-2.69 (m, 7H), 2.65-2.33 (m, 9H), 2.37-2.34 (m, 1H), 2.29 (s, 3H), 1.75-1.65 (m, 1H).

### Example 71: N-[(3R,5S)-1-(8-cyanoquinazolin-5-yl)-5-(trifluoromethyl)piperidin-3-yl]-2-(4-fluoro-1-methylpiperidin-4-yl)acetamide

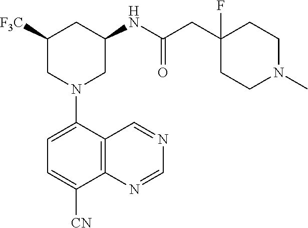 [SMILES: CN1CCC(F)(CC(=O)N[C@@H]2C[C@H](C(F)(F)F)CN(c3ccc(C#N)c4ncncc34)C2)CC1]

The title compound was prepared from 5-[(3R,5S)-3-amino-5-(trifluoromethyl)piperidin-1-yl]quinazoline-8-carbonitrile and 2-(4-fluoro-1-methylpiperidin-4-yl)acetic acid. MS: 479 [M+H]⁺. ¹H NMR (400 MHz, Methanol-d₄, ppm) δ 9.73 (s, 1H), 9.32 (s, 1H), 8.31 (d, J=8.2 Hz, 1H), 7.37 (d, J=8.2 Hz, 1H), 4.38-4.26 (m, 1H), 3.90-3.77 (m, 2H), 3.17-3.06 (m, 2H), 2.83-2.65 (m, 3H), 2.59-2.50 (m, 2H), 2.41-2.30 (m, 3H), 2.28 (s, 3H), 1.99-1.76 (m, 4H), 1.72-1.57 (m, 1H).

### Example 72: N-[(3R,5S)-1-(8-cyanoquinoxalin-5-yl)-5-methylpiperidin-3-yl]-2-(4-hydroxy-1-methylpiperidin-4-yl)acetamide hydrochloride

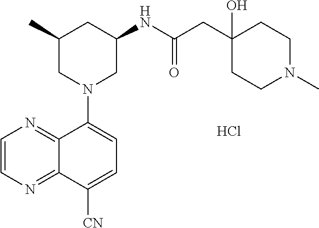 [SMILES: C[C@H]1C[C@@H](NC(=O)CC2(O)CCN(C)CC2)CN(c2ccc(C#N)c3nccnc23)C1 ; Cl]

The title compound was prepared from 8-[(3R,5S)-3-amino-5-methylpiperidin-1-yl]quinoxaline-5-carbonitrile and 2-(4-hydroxy-1-methylpiperidin-4-yl)acetic acid. MS: 423 [M+H]⁺. ¹H NMR (300 MHz, Methanol-d₄, ppm) δ 8.93 (d, J=1.8 Hz, 1H), 8.88 (d, J=1.8 Hz, 1H), 8.09 (d, J=8.4 Hz, 1H), 7.29 (d, J=8.4 Hz, 1H), 4.43-4.26 (m, 2H), 4.23-4.06 (m, 1H), 3.39-3.31 (m, 2H), 2.88 (s, 3H), 2.84-2.62 (m, 2H), 2.45 (s, 2H), 2.17-1.78 (m, 7H), 1.40-1.14 (m, 2H), 1.01 (d, J=6.4 Hz, 3H).

### Example 73: N-[(3R,5S)-1-(8-cyanoquinolin-5-yl)-5-(trifluoromethyl)piperidin-3-yl]-2-(4-hydroxy-1-methylpiperidin-4-yl)acetamide

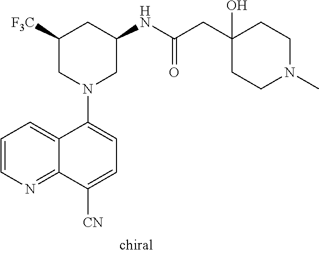 [SMILES: CN1CCC(O)(CC(=O)N[C@@H]2C[C@H](C(F)(F)F)CN(c3ccc(C#N)c4ncccc34)C2)CC1]

The title compound was prepared from 8-[(3R,5S)-3-amino-5-trifluoromethylpiperidin-1-yl]quinoxaline-5-carbonitrile and 2-(4-hydroxy-1-methylpiperidin-4-yl)acetic acid. MS: 467 [M+H]⁺. ¹H NMR (400 MHz, Methanol-d₄, ppm) δ 9.05-8.98 (m, 1H), 8.67 (dd, J=8.6, 1.7 Hz, 1H), 8.16 (d, J=8.0 Hz, 1H), 7.70 (dd, J=8.6, 4.2 Hz, 1H), 7.32 (d, J=8.0 Hz, 1H), 4.39-4.27 (m, 1H), 3.80-3.70 (m, 1H), 3.70-3.63 (m, 1H), 3.13-2.92 (m, 2H), 2.68-2.53 (m, 3H), 2.48-2.37 (m, 2H), 2.36 (s, 3H), 2.28 (s, 3H), 1.78-1.53 (m, 5H).

### Example 74: N-[(3R,5S)-1-(8-cyanoquinoxalin-5-yl)-5-methylpiperidin-3-yl]-2-(3-methyl-1,2-oxazol-5-yl)acetamide

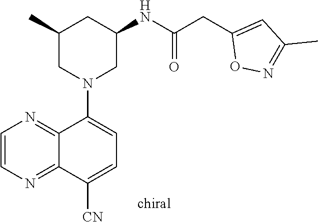 [SMILES: Cc1cc(CC(=O)N[C@@H]2C[C@H](C)CN(c3ccc(C#N)c4nccnc34)C2)on1]

The title compound was prepared from 8-[(3R,5S)-3-amino-5-methylpiperidin-1-yl]quinoxaline-5-carbonitrile and 2-(3-methyl-1,2-oxazol-5-yl)acetic acid. MS: 391 [M+H]⁺. ¹H NMR (300 MHz, DMSO-d₆, ppm) δ 9.04 (d, J=1.8 Hz, 1H), 8.96 (d, J=1.8 Hz, 1H), 8.33 (d, J=7.3 Hz, 1H), 8.21 (d, J=8.4 Hz, 1H), 7.29 (d, J=8.5 Hz, 1H), 6.21 (s, 1H), 4.44-4.21 (m, 2H), 3.95-3.89 (m, 1H), 3.67 (s, 2H), 2.85-2.64 (m, 2H), 2.21 (s, 3H), 2.06-1.82 (m, 2H), 1.27-1.09 (m, 1H), 0.94 (d, J=6.5 Hz, 3H).

### Example 75: N-[(3R,5S)-1-(8-cyanoquinoxalin-5-yl)-5-(trifluoromethyl)piperidin-3-yl]-3-(dimethylamino)propenamide

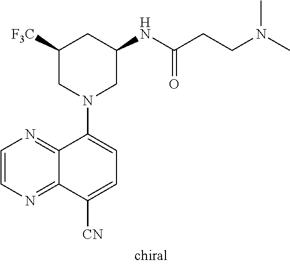 [SMILES: CN(C)CCC(=O)N[C@@H]1C[C@H](C(F)(F)F)CN(c2ccc(C#N)c3nccnc23)C1]

The title compound was prepared from 8-[(3R,5S)-3-amino-5-methylpiperidin-1-yl]quinoxaline-5-carbonitrile and 3-(dimethylamino)propanoic acid. MS: 421 [M+H]⁺. ¹H NMR (400 MHz, Methanol-d₄, ppm) δ 8.96-8.91 (m, 2H), 8.14 (d, J=8.3 Hz, 1H), 7.35 (d, J=8.4 Hz, 1H), 4.72-4.55 (m, 1H), 4.36-4.16 (m, 2H), 3.12-2.80 (m, 3H), 2.68 (t, J=7.3 Hz, 2H), 2.43 (t, J=7.6, 6.5 Hz, 2H), 2.39-2.32 (m, 1H), 2.30 (s, 6H), 1.69-1.55 (m, 1H).

### Example 76: N-[(3R,5S)-1-(8-cyanoquinoxalin-5-yl)-5-(trifluoromethyl)piperidin-3-yl]-2-(4-hydroxy-1-methylpiperidin-4-yl)acetamide

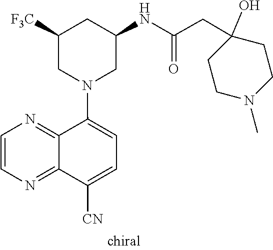 [SMILES: CN1CCC(O)(CC(=O)N[C@@H]2C[C@H](C(F)(F)F)CN(c3ccc(C#N)c4nccnc34)C2)CC1]

The title compound was prepared from 8-[(3R,5S)-3-amino-5-trifluoromethylpiperidin-1-yl]quinoxaline-5-carbonitrile and 2-(4-hydroxy-1-methylpiperidin-4-yl)acetic acid. MS: 477 [M+H]⁺. ¹H NMR (400 MHz, Methanol-d₄, ppm) δ 8.96 (d, J=1.8 Hz, 1H), 8.92 (d, J=1.8 Hz, 1H), 8.13 (d, J=8.3 Hz, 1H), 7.34 (d, J=8.4 Hz, 1H), 4.71-4.63 (m, 1H), 4.31-4.16 (m, 2H), 3.16-2.82 (m, 3H), 2.69-2.62 (m, 2H), 2.57-2.46 (m, 2H), 2.39 (s, 2H), 2.37-2.35 (m, 1H), 2.34 (s, 3H), 1.83-1.68 (m, 4H), 1.71-1.57 (m, 1H).

### Example 77: 2-(4-fluoro-1-methylpiperidin-4-yl)-N-[(3R,5S)-5-methyl-1-[8-(trifluoromethyl)quinolin-5-yl]piperidin-3-yl]acetamide

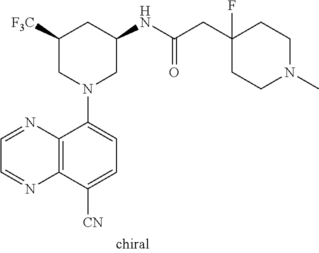 [SMILES: CN1CCC(F)(CC(=O)N[C@@H]2C[C@H](C(F)(F)F)CN(c3ccc(C#N)c4nccnc34)C2)CC1]

The title compound was prepared from 8-[(3R,5S)-3-amino-5-methylpiperidin-1-yl]quinoxaline-5-carbonitrile and 2-(4-fluoro-1-methylpiperidin-4-yl)acetic acid. MS: 479 [M+H]⁺. ¹H NMR (400 MHz, Methanol-d₄, ppm) δ 8.96 (d, J=1.8 Hz, 1H), 8.92 (d, J=1.8 Hz, 1H), 8.13 (d, J=8.3 Hz, 1H), 7.33 (d, J=8.3 Hz, 1H), 4.69-4.61 (m, 1H), 4.34-4.16 (m, 2H), 3.06 (t, J=11.7 Hz, 1H), 2.99-2.82 (m, 3H), 2.70-2.55 (m, 4H), 2.49 (s, 3H), 2.40-2.31 (m, 1H), 2.13-1.89 (m, 4H), 1.71-1.57 (m, 1H), 1.49-1.40 (m, 1H).

### Example 78: 3-(dimethylamino)-N-[(3R,5S)-5-methyl-1-[8-(trifluoromethyl)quinoxalin-5-yl]piperidin-3-yl]propenamide

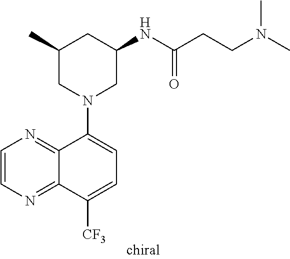 [SMILES: C[C@H]1C[C@@H](NC(=O)CCN(C)C)CN(c2ccc(C(F)(F)F)c3nccnc23)C1]

The title compound was prepared from (3R,5S)-5-methyl-1-[8-(trifluoromethyl)quinoxalin-5-yl]piperidin-3-amine and 3-(dimethylamino)propanoic acid. MS: 410 [M+H]⁺. ¹H NMR (400 MHz, Methanol-d₄, ppm) δ 8.94-8.87 (m, 2H), 8.04 (d, J=8.4 Hz, 1H), 7.29 (d, J=8.3 Hz, 1H), 4.59 (br s, 1H), 4.28-4.07 (m, 3H), 2.71-2.62 (m, 3H), 2.62-2.52 (m, 1H), 2.45-2.37 (m, 2H), 2.29 (s, 6H), 2.17-2.02 (m, 2H), 1.23-1.10 (m, 1H), 1.02 (d, J=6.4 Hz, 3H).

### Example 79: N-((3R,5S)-1-(8-cyanoquinoxalin-5-yl)-5-methylpiperidin-3-yl)-3-(dimethylamino)propenamide

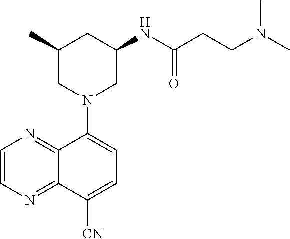 [SMILES: C[C@H]1C[C@@H](NC(=O)CCN(C)C)CN(c2ccc(C#N)c3nccnc23)C1]

The title compound was prepared from 8-[(3R,5S)-3-amino-5-methylpiperidin-1-yl]quinoxaline-5-carbonitrile and 3-(dimethylamino)propanoic acid. MS: 367 [M+H]⁺. ¹H NMR (300 MHz, Methanol-d₄, ppm) δ 8.93-8.88 (m, 2H), 8.09 (d, J=8.4 Hz, 1H), 7.29 (d, J=8.4 Hz, 1H), 4.35 (dd, J=23.1, 10.0 Hz, 2H), 4.17-4.11 (m, 1H), 2.81-2.60 (m, 4H), 2.46-2.35 (m, 2H), 2.29 (s, 6H), 2.16-2.0 (m, 2H), 1.33-1.11 (m, 1H), 1.01 (d, J=6.5 Hz, 3H).

### Example 80: 2-(4-hydroxy-1-methylpiperidin-4-yl)-N-[(3R,5S)-5-methyl-1-[8-(trifluoromethyl)quinoxalin-5-yl]piperidin-3-yl]acetamide hydrochloride

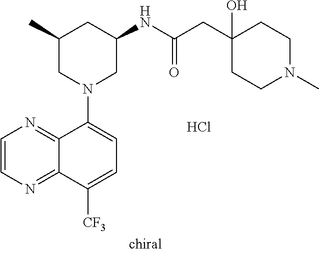 [SMILES: C[C@H]1C[C@@H](NC(=O)CC2(O)CCN(C)CC2)CN(c2ccc(C(F)(F)F)c3nccnc23)C1 ; Cl]

The title compound was prepared from (3R,5S)-5-methyl-1-[8-(trifluoromethyl)quinoxalin-5-yl]piperidin-3-amine and 2-(4-hydroxy-1-methylpiperidin-4-yl)acetic acid. MS: 466 [M+H]⁺. ¹H NMR (300 MHz, Methanol-d₄, ppm) δ 9.10-9.0 (m, 2H), 8.22 (d, J=8.3 Hz, 1H), 7.84 (d, J=8.3 Hz, 1H), 4.47-4.32 (m, 1H), 4.19-4.09 (m, 1H), 4.04-3.95 (m, 1H), 3.57-3.42 (m, 2H), 3.36-3.30 (m, 1H), 3.21-2.98 (m, 3H), 2.85 (s, 3H), 2.45 (s, 2H), 2.32-2.23 (m, 1H), 2.21-2.11 (m, 1H), 2.05-1.84 (m, 4H), 1.43-1.30 (m, 1H), 1.07 (d, J=6.6 Hz, 3H).

### Example 81: N-[(3R,5S)-1-(8-cyanoquinolin-5-yl)-5-(trifluoromethyl)piperidin-3-yl]-2-(4-hydroxy-1-methylpiperidin-4-yl)acetamide

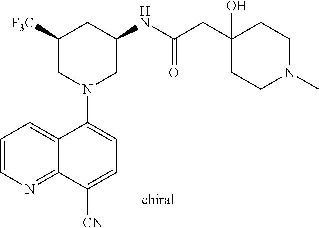 [SMILES: CN1CCC(O)(CC(=O)N[C@@H]2C[C@H](C(F)(F)F)CN(c3ccc(C#N)c4ncccc34)C2)CC1]

The title compound was prepared from 5-[(3R,5S)-3-amino-5-(trifluoromethyl)piperidin-1-yl]quinoline-8-carbonitrile and 2-(4-hydroxy-1-methylpiperidin-4-yl)acetic acid. MS: 467 [M+H]⁺. ¹H NMR (400 MHz, Methanol-d₄, ppm) δ 9.05-8.98 (m, 1H), 8.67 (dd, J=8.6, 1.7 Hz, 1H), 8.16 (d, J=8.0 Hz, 1H), 7.70 (dd, J=8.6, 4.2 Hz, 1H), 7.32 (d, J=8.0 Hz, 1H), 4.39-4.27 (m, 1H), 3.80-3.70 (m, 1H), 3.70-3.63 (m, 1H), 3.13-2.92 (m, 2H), 2.68-2.53 (m, 3H), 2.48-2.37 (m, 2H), 2.36 (s, 3H), 2.28 (s, 3H), 1.78-1.53 (m, 5H).

### Example 82: N-((3R,5S)-1-(8-cyanoquinolin-5-yl)-5-(trifluoromethyl)piperidin-3-yl)-2-(4-methylpiperazin-1-yl)acetamide

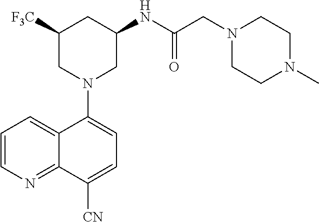 [SMILES: CN1CCN(CC(=O)N[C@@H]2C[C@H](C(F)(F)F)CN(c3ccc(C#N)c4ncccc34)C2)CC1]

The title compound was prepared from 5-[(3R,5S)-3-amino-5-(trifluoromethyl)piperidin-1-yl]quinoline-8-carbonitrile 2-(4-methylpiperazin-1-yl)acetic acid. MS: 461 [M+H]⁺. ¹H NMR (400 MHz, Methanol-d₄, ppm) δ 9.01 (dd, J=4.3, 1.7 Hz, 1H), 8.68 (dd, J=8.6, 1.7 Hz, 1H), 8.17 (d, J=8.0 Hz, 1H), 7.70 (dd, J=8.6, 4.3 Hz, 1H), 7.33 (d, J=8.0 Hz, 1H), 4.42-4.31 (m, 1H), 3.70-3.65 (m, 2H), 3.07 (s, 2H), 3.05-2.95 (m, 2H), 2.78-2.43 (m, 9H), 2.39-2.32 (m, 1H), 2.30 (s, 3H), 1.77-1.63 (m, 1H).

### Example 83: 2-(4-fluoro-1-methylpiperidin-4-yl)-N-[(3R,5S)-5-methyl-1-[8-(trifluoromethyl)quinolin-5-yl]piperidin-3-yl]acetamide

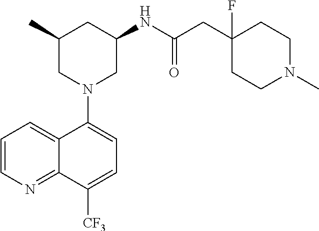 [SMILES: C[C@H]1C[C@@H](NC(=O)CC2(F)CCN(C)CC2)CN(c2ccc(C(F)(F)F)c3ncccc23)C1]

The title compound was from (3R,5S)-5-methyl-1-[8-(trifluoromethyl)quinolin-5-yl]piperidin-3-amine and 2-(4-fluoro-1-methylpiperidin-4-yl)acetic acid. MS: 468 [M+H]⁺. ¹H NMR (400 MHz, Methanol-d₄, ppm) δ 8.97-8.91 (m, 1H), 8.66 (dd, J=8.6, 1.8 Hz, 1H), 8.04 (d, J=8.0 Hz, 1H), 7.63 (dd, J=8.6, 4.2 Hz, 1H), 7.23 (d, J=8.0 Hz, 1H), 4.26-4.22 (m, 1H), 3.68-3.60 (m, 1H), 3.43-3.36 (m, 1H), 2.73-2.65 (m, 2H), 2.58-2.44 (m, 4H), 2.35-2.31 (m, 2H), 2.29 (s, 3H), 2.22-2.08 (m, 2H), 2.0-1.77 (m, 4H), 1.24-1.10 (m, 1H), 1.05 (d, J=6.4 Hz, 3H).

### Example 84: 2-(4-hydroxy-1-methylpiperidin-4-yl)-N-[(3R,5S)-5-methyl-1-[8-(trifluoromethyl)quinolin-5-yl]piperidin-3-yl]acetamide

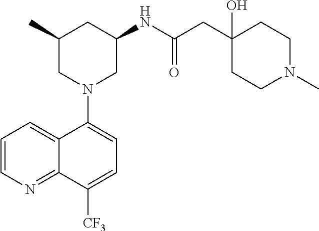 [SMILES: C[C@H]1C[C@@H](NC(=O)CC2(O)CCN(C)CC2)CN(c2ccc(C(F)(F)F)c3ncccc23)C1]

The title compound was prepared from (3R,5S)-5-methyl-1-[8-(trifluoromethyl)quinolin-5-yl]piperidin-3-amine and 2-(4-hydroxy-1-methylpiperidin-4-yl)acetic acid. MS: 465 [M+H]⁺. ¹H NMR (400 MHz, Methanol-d₄, ppm) δ 8.94 (dd, J=4.2, 1.8 Hz, 1H), 8.66 (dd, J=8.6, 1.8 Hz, 1H), 8.04 (d, J=8.1 Hz, 1H), 7.63 (dd, J=8.6, 4.2 Hz, 1H), 7.23 (d, J=8.0 Hz, 1H), 4.30-4.19 (m, 1H), 3.70-3.62 (m, 1H), 3.43-3.36 (m, 1H), 2.64-2.56 (m, 2H), 2.56-2.40 (m, 4H), 2.35 (s, 2H), 2.29 (s, 3H), 2.25-2.11 (m, 2H), 1.78-1.62 (m, 4H), 1.24-1.10 (m, 1H), 1.08-1.02 (m, 3H).

### Example 85: 2-(4-hydroxy-1-methylpiperidin-4-yl)-N-[(3R,5S)-5-methyl-1-(8-methylquinolin-5-yl)piperidin-3-yl]acetamide hydrochloride

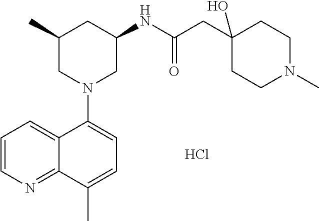 [SMILES: Cc1ccc(N2C[C@@H](C)C[C@@H](NC(=O)CC3(O)CCN(C)CC3)C2)c2cccnc12 ; Cl]

The title compound was prepared (3R,5S)-5-methyl-1-(8-methylquinolin-5-yl)piperidin-3-amine and 2-(4-hydroxy-1-methylpiperidin-4-yl)acetic acid. MS: 411 [M+H]⁺. ¹H NMR (400 MHz, DMSO-d₆, ppm) δ 10.15 (br s, 1H), 9.19-9.13 (m, 1H), 9.09-9.02 (m, 1H), 8.18 (d, J=7.4 Hz, 1H), 8.03-7.95 (m, 1H), 7.82 (d, J=7.7 Hz, 1H), 7.37 (d, J=8.1 Hz, 1H), 4.07 (d, J=10.6 Hz, 1H), 3.45-3.37 (m, 1H), 3.26-3.17 (m, 3H), 3.15-3.01 (m, 2H), 2.76-2.68 (m, 6H), 2.51-2.36 (m, 2H), 2.31 (s, 2H), 2.17-1.82 (m, 4H), 1.73-1.65 (m, 2H), 1.18-1.03 (m, 1H), 0.95 (d, J=6.4 Hz, 3H).

### Example 86: N-[(3R,5S)-5-methyl-1-(8-methylquinolin-5-yl)piperidin-3-yl]-2-(4-methylpiperazin-1-yl)acetamide

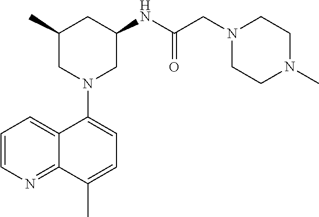 [SMILES: Cc1ccc(N2C[C@@H](C)C[C@@H](NC(=O)CN3CCN(C)CC3)C2)c2cccnc12]

The title compound was prepared from (3R,5S)-5-methyl-1-(8-methylquinolin-5-yl)piperidin-3-amine and 2-(4-methylpiperazin-1-yl)acetic acid. MS: 396 [M+H]⁺. ¹H NMR (300 MHz, DMSO-d₆, ppm) δ 8.90 (dd, J=4.1, 1.8 Hz, 1H), 8.46 (dd, J=8.5, 1.8 Hz, 1H), 7.62-7.46 (m, 3H), 7.08 (d, J=7.6 Hz, 1H), 4.11-4.03 (m, 1H), 3.33-3.23 (m, 1H), 3.20-3.10 (m, 1H), 2.89 (s, 2H), 2.64 (s, 3H), 2.47-2.24 (m, 10H), 2.14 (s, 3H), 2.09-1.86 (m, 2H), 1.22-1.04 (m, 1H), 0.94 (d, J=6.5 Hz, 3H).

### Example 87: 2-(4-hydroxy-1-methylpiperidin-4-yl)-N-[(3R,5S)-1-(8-methylquinolin-5-yl)-5-(trifluoromethyl)piperidin-3-yl]acetamide

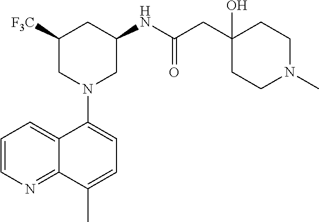 [SMILES: Cc1ccc(N2C[C@H](NC(=O)CC3(O)CCN(C)CC3)C[C@H](C(F)(F)F)C2)c2cccnc12]

The title compound was prepared from (3R,5S)-1-(8-methylquinolin-5-yl)-5-(trifluoromethyl)piperidin-3-amine and 2-(4-hydroxy-1-methylpiperidin-4-yl)acetic acid. MS: 465 [M+H]⁺. ¹H NMR (300 MHz, Methanol-d₄, ppm) δ 8.86 (dd, J=4.3, 1.7 Hz, 1H), 8.65 (dd, J=8.6, 1.7 Hz, 1H), 7.61-7.49 (m, 2H), 7.19 (d, J=7.6 Hz, 1H), 4.35-4.22 (m, 1H), 3.52-3.37 (m, 2H), 3.04-2.93 (m, 2H), 2.89-2.75 (m, 1H), 2.69 (s, 3H), 2.62-2.29 (m, 7H), 2.25 (s, 3H), 1.78-1.63 (m, 4H), 1.59-1.41 (m, 1H).

### Example 88: N-[(3R,5S)-1-(8-cyanoquinazolin-5-yl)-5-methylpiperidin-3-yl]-2-(4-fluoro-1-methylpiperidin-4-yl)acetamide

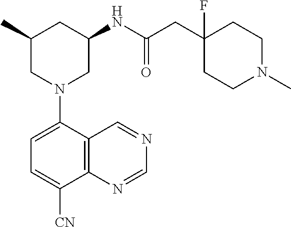

The title compound was prepared from 5-[(3R,5S)-3-amino-5-methylpiperidin-1-yl]quinazoline-8-carbonitrile and 2-(4-fluoro-1-methylpiperidin-4-yl)acetic acid. MS: 425 [M+H]⁺. ¹H NMR (400 MHz, Methanol-d₄, ppm) δ 9.68 (s, 1H), 9.30 (s, 1H), 8.28 (d, J=8.3 Hz, 1H), 7.30 (d, J=8.3 Hz, 1H), 4.26-4.14 (m, 1H), 3.95-3.87 (m, 1H), 3.70-3.63 (m, 1H), 3.10-3.06 (m, 2H), 2.93-2.67 (m, 4H), 2.65-2.56 (m, 5H), 2.24-1.84 (m, 6H), 1.31-1.17 (m, 1H), 1.05 (d, J=6.4 Hz, 3H).

### Example 89: N-[(3R,5S)-1-(8-cyanoquinazolin-5-yl)-5-methylpiperidin-3-yl]-2-(4-methylpiperazin-1-yl)acetamide

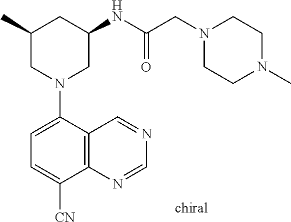

The title compound was prepared from 5-[(3R,5S)-3-amino-5-methylpiperidin-1-yl]quinazoline-8-carbonitrile and 2-(4-methylpiperazin-1-yl)acetic acid. MS: 408 [M+H]⁺. ¹H NMR (400 MHz, DMSO-d₆, ppm) δ 9.60 (s, 1H), 9.37 (s, 1H), 8.37 (d, J=8.3 Hz, 1H), 7.77 (d, J=7.7 Hz, 1H), 7.25 (d, J=8.4 Hz, 1H), 4.07-4.03 (m, 1H), 3.79-3.72 (m, 1H), 3.66-3.59 (m, 1H), 2.97 (s, 2H), 2.82-2.52 (m, 9H), 2.38-2.34 (m, 3H), 2.13-1.91 (m, 2H), 1.33-1.20 (m, 2H), 0.95 (d, J=6.5 Hz, 3H).

### Example 90: (2R)—N-[(3R,5S)-1-(8-cyanoquinoxalin-5-yl)-5-methylpiperidin-3-yl]-2-(4-methylpiperazin-1-yl)propanamide & Example 91: (2S)—N-[(3R,5S)-1-(8-cyanoquinoxalin-5-yl)-5-methylpiperidin-3-yl]-2-(4-methylpiperazin-1-yl)propanamide

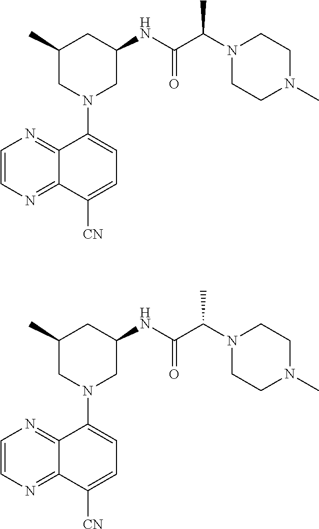 [SMILES: C[C@H]1C[C@@H](NC(=O)[C@@H](C)N2CCN(C)CC2)CN(c2ccc(C#N)c3nccnc23)C1 ; C[C@H]1C[C@@H](NC(=O)[C@H](C)N2CCN(C)CC2)CN(c2ccc(C#N)c3nccnc23)C1]

The title compounds were prepared from 8-[(3R,5S)-3-amino-5-methylpiperidin-1-yl]quinoxaline-5-carbonitrile and 2-(4-methylpiperazin-1-yl)propanoic acid, followed by separation on chiral-HPLC under the following conditions: column Repaired CHIRALPAK IC-3, 0.46×10 cm, 3 um; mobile phase, MtBE (with 0.1% DEA) in EtOH, 70% isocratic in 20 min; detector, UV 220 nm. (chirality of 2-(4-methylpiperazin-1-yl)propanamide was assigned arbitrarily).

### Example 90

MS: 422 [M+H]⁺. ¹H NMR (300 MHz, Methanol-d₄, ppm) δ 8.92 (d, J=1.8 Hz, 1H), 8.88 (d, J=1.8 Hz, 1H), 8.08 (d, J=8.4 Hz, 1H), 7.28 (d, J=8.4 Hz, 1H), 4.69-4.52 (m, 1H), 4.34-4.23 (m, 2H), 4.21-4.05 (m, 1H), 3.08-2.96 (m, 1H), 2.88-2.75 (m, 1H), 2.76-2.39 (m, 8H), 2.28 (s, 3H), 2.15-1.94 (m, 2H), 1.35-1.16 (m, 4H), 1.01 (d, J=6.4 Hz, 3H).

### Example 91

MS: 422 [M+H]⁺. ¹H NMR (400 MHz, Methanol-d₄, ppm) δ 8.91 (d, J=1.8 Hz, 1H), 8.88 (d, J=1.8 Hz, 1H), 8.10 (d, J=8.4 Hz, 1H), 7.30 (d, J=8.4 Hz, 1H), 4.62-4.58 (m, 1H), 4.40-4.25 (m, 2H), 4.21-4.09 (m, 1H), 3.09-2.99 (m, 1H), 2.88-2.78 (m, 1H), 2.76-2.44 (m, 8H), 2.29 (s, 3H), 2.14-1.92 (m, 2H), 1.34-1.21 (m, 4H), 1.03 (d, J=6.5 Hz, 3H).

### Example 92: (2R)—N-[(3R,5S)-5-methyl-1-[8-(trifluoromethyl)quinolin-5-yl]piperidin-3-yl]-2-(4-methylpiperazin-1-yl)propanamide & Example 93: (2S)—N-[(3R,5S)-5-methyl-1-[8-(trifluoromethyl)quinolin-5-yl]piperidin-3-yl]-2-(4-methylpiperazin-1-yl)propanamide

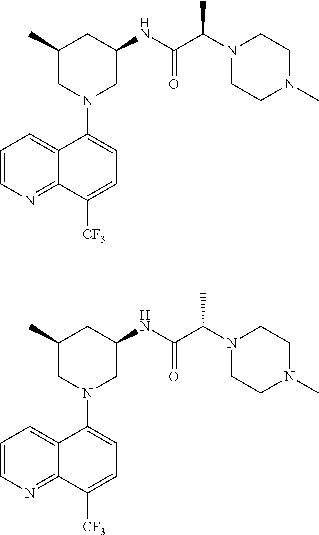 [SMILES: C[C@H]1C[C@@H](NC(=O)[C@@H](C)N2CCN(C)CC2)CN(c2ccc(C(F)(F)F)c3ncccc23)C1 ; C[C@H]1C[C@@H](NC(=O)[C@H](C)N2CCN(C)CC2)CN(c2ccc(C(F)(F)F)c3ncccc23)C1]

The title compounds were prepared from (3R,5S)-5-methyl-1-[8-(trifluoromethyl)quinolin-5-yl]piperidin-3-amine and 2-(4-methylpiperazin-1-yl)propanoic acid, followed by separation on chiral-HPLC under the following conditions: column, CHIRALPAK ADH, 0.46×10 cm, 3 um; mobile phase, hexane (with 0.1% DEA) in EtOH, 90% isocratic in 20 min; detector, UV 220 nm. (chirality of 2-(4-methylpiperazin-1-yl)propanamide was assigned arbitrarily).

### Example 92

MS: 464 [M+H]⁺. ¹H NMR (400 MHz, Methanol-d₄, ppm) δ 8.94 (dd, J=4.3, 1.8 Hz, 1H), 8.67 (dd, J=8.6, 1.8 Hz, 1H), 8.04 (d, J=8.2 Hz, 1H), 7.63 (dd, J=8.6, 4.3 Hz, 1H), 7.24 (d, J=8.0 Hz, 1H), 4.26-4.22 (m, 1H), 3.67-3.60 (m, 1H), 3.43-3.36 (m, 1H), 3.09-3.0 (m, 1H), 2.76-2.38 (m, 10H), 2.29 (s, 3H), 2.23-2.04 (m, 2H), 1.28-1.14 (m, 4H), 1.06 (d, J=6.5 Hz, 3H).

### Example 93

MS: 464 [M+H]⁺. ¹H NMR (400 MHz, Methanol-d₄, ppm) δ 8.94 (dd, J=4.3, 1.8 Hz, 1H), 8.67 (dd, J=8.6, 1.8 Hz, 1H), 8.04 (d, J=8.2 Hz, 1H), 7.62 (dd, J=8.6, 4.3 Hz, 1H), 7.24 (d, J=8.0 Hz, 1H), 4.26-4.22 (m, 1H), 3.67-3.60 (m, 1H), 3.43-3.36 (m, 1H), 3.09-3.0 (m, 1H), 2.76-2.38 (m, 10H), 2.29 (s, 3H), 2.23-2.04 (m, 2H), 1.28-1.14 (m, 4H), 1.05 (d, J=6.5 Hz, 3H).

### Example 94: (2R)-2-(4-methylpiperazin-1-yl)-N-[(3R,5S)-5-(trifluoromethyl)-1-[8-(trifluoromethyl)quinolin-5-yl]piperidin-3-yl]propanamide & Example 95: (2S)-2-(4-methylpiperazin-1-yl)-N-[(3R,5S)-5-(trifluoromethyl)-1-[8-(trifluoromethyl)quinolin-5-yl]piperidin-3-yl]propanamide

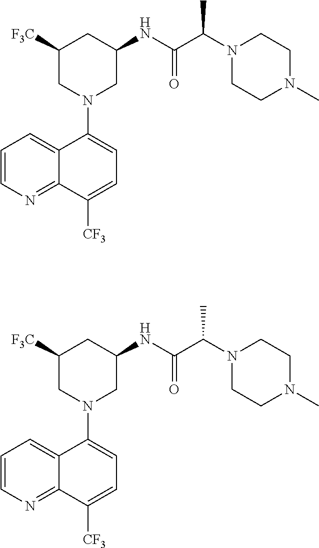 [SMILES: C[C@H](C(=O)N[C@@H]1C[C@H](C(F)(F)F)CN(c2ccc(C(F)(F)F)c3ncccc23)C1)N1CCN(C)CC1 ; C[C@@H](C(=O)N[C@@H]1C[C@H](C(F)(F)F)CN(c2ccc(C(F)(F)F)c3ncccc23)C1)N1CCN(C)CC1]

The title compounds were prepared from (3R,5S)-5-(trifluoromethyl)-1-[8-(trifluoromethyl)quinolin-5-yl]piperidin-3-amine and 2-(4-methylpiperazin-1-yl)propanoic acid, followed by separation on chiral-HPLC under the following conditions: column, CHIRALPAK IC-3, 0.46×5 cm, 3 um; mobile phase, hexane (with 0.1% DEA) in EtOH, 93% isocratic in 20 min; detector, UV 254 nm. (chirality of 2-(4-methylpiperazin-1-yl)propanamide was assigned arbitrarily).

### Example 94

MS: 518 [M+H]⁺. ¹H NMR (400 MHz, Methanol-d₄, ppm) δ 8.97 (dd, J=4.3, 1.7 Hz, 1H), 8.68 (dd, J=8.6, 1.8 Hz, 1H), 8.08 (d, J=8.2 Hz, 1H), 7.67 (dd, J=8.6, 4.2 Hz, 1H), 7.32 (d, J=8.0 Hz, 1H), 4.34 (m, J=15.5, 10.8, 4.2 Hz, 1H), 3.60 (d, J=11.1 Hz, 2H), 3.06 (m, J=6.9 Hz, 2H), 2.96 (m, J=11.2 Hz, 1H), 2.84-2.17 (m, 13H), 1.65 (m, J=12.3 Hz, 1H), 1.24 (d, J=6.9 Hz, 3H).

### Example 95

MS: 518 [M+H]⁺. ¹H NMR (400 MHz, Methanol-d₄, ppm) δ 9.0-8.94 (m, 1H), 8.68 (dd, J=8.6, 1.8 Hz, 1H), 8.08 (d, J=8.1 Hz, 1H), 7.67 (dd, J=8.6, 4.2 Hz, 1H), 7.32 (d, J=8.0 Hz, 1H), 4.41-4.28 (m, 1H), 3.64-3.57 (m, 2H), 3.14-3.0 (m, 2H), 2.96 (t, J=11.2 Hz, 1H), 2.76-2.45 (m, 9H), 2.40-2.35 (m, 1H), 2.34 (s, 3H), 1.72-1.59 (m, 1H), 1.24 (d, J=6.9 Hz, 3H).

### Example 96: (2R)—N-[(3R,5S)-5-amino-1-(8-cyanoquinazolin-5-yl)piperidin-3-yl]-2-(4-methylpiperazin-1-yl)propanamide & Example 97: (2S)—N-[(3R,5S)-5-amino-1-(8-cyanoquinazolin-5-yl)piperidin-3-yl]-2-(4-methylpiperazin-1-yl)propanamide

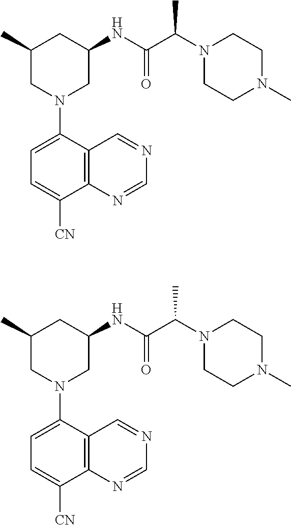

The title compounds were prepared from 5-[(3R,5S)-3-amino-5-methylpiperidin-1-yl]quinazoline-8-carbonitrile and 2-(4-methylpiperazin-1-yl)propanoic acid, followed by separation on chiral-HPLC under the following conditions: column, Repaired Chiral Cellulose-SB, 0.46×10 cm, 3 um; mobile phase, hexane (20 mmol NH₃) in EtOH, 70% isocratic in 20 min; detector, UV 254 nm. (chirality of 2-(4-methylpiperazin-1-yl)propanamide was assigned arbitrarily).

### Example 96

MS: 422 [M+H]⁺. ¹H NMR (400 MHz, DMSO-d₆, ppm) δ 9.61 (s, 1H), 9.37 (s, 1H), 8.38 (d, J=8.3 Hz, 1H), 7.72 (d, J=7.5 Hz, 1H), 7.25 (d, J=8.4 Hz, 1H), 4.07-3.99 (m, 1H), 3.81-3.73 (m, 1H), 3.66-3.58 (m, 1H), 3.06-2.96 (m, 1H), 2.80-2.66 (m, 2H), 2.49-2.19 (m, 8H), 2.13 (s, 3H), 2.09-1.89 (m, 2H), 1.32-1.18 (m, 1H), 1.08 (d, J=6.9 Hz, 3H), 0.95 (d, J=6.5 Hz, 3H).

### Example 97

MS: 422 [M+H]⁺. ¹H NMR (400 MHz, DMSO-d₆, ppm) δ 9.61 (s, 1H), 9.36 (s, 1H), 8.37 (d, J=8.3 Hz, 1H), 7.71 (d, J=7.6 Hz, 1H), 7.25 (d, J=8.4 Hz, 1H), 4.08-3.98 (m, 1H), 3.75 (d, J=12.7 Hz, 1H), 3.63 (d, J=11.7 Hz, 1H), 3.01 (q, J=6.9 Hz, 1H), 2.72 (dt, J=23.2, 11.5 Hz, 2H), 2.44 (d, J=14.7 Hz, 4H), 2.33 (s, 4H), 2.15 (s, 3H), 2.05 (s, 1H), 1.97 (d, J=12.8 Hz, 1H), 1.25 (q, J=12.0 Hz, 1H), 1.08 (d, J=6.9 Hz, 3H), 0.95 (d, J=6.5 Hz, 3H).

### Example 98: (2R)—N-[(3R,5S)-1-(8-cyanoquinoxalin-5-yl)-5-(trifluoromethyl)piperidin-3-yl]-2-(4-methylpiperazin-1-yl)propanamide & Example 99: (2S)—N-[(3R,5S)-1-(8-cyanoquinoxalin-5-yl)-5-(trifluoromethyl)piperidin-3-yl]-2-(4-methylpiperazin-1-yl)propenamide

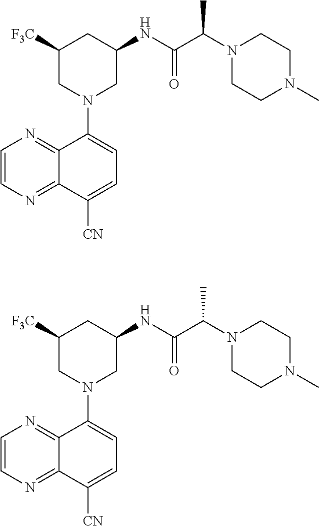 [SMILES: C[C@H](C(=O)N[C@@H]1C[C@H](C(F)(F)F)CN(c2ccc(C#N)c3nccnc23)C1)N1CCN(C)CC1 ; C[C@@H](C(=O)N[C@@H]1C[C@H](C(F)(F)F)CN(c2ccc(C#N)c3nccnc23)C1)N1CCN(C)CC1]

The title compounds were prepared from 8-[(3R,5S)-3-amino-5-(trifluoromethyl)piperidin-1-yl]quinoxaline-5-carbonitrile and 2-(4-methylpiperazin-1-yl)propanoic acid, followed by separation on chiral-HPLC under the following conditions: column, Repaired Chiral-ADH, 0.46×10 cm, 3 um; mobile phase, hexane (0.2% IPA) in EtOH, 85% isocratic in 20 min; detector, UV 220 nm. (chirality of 2-(4-methylpiperazin-1-yl)propanamide was assigned arbitrarily).

### Example 98

MS: 476 [M+H]⁺. ¹H NMR (400 MHz, Methanol-d₄, ppm) δ 9.0-8.91 (m, 2H), 8.15 (d, J=8.3, 1.1 Hz, 1H), 7.35 (d, J=8.4 Hz, 1H), 4.68-4.60 (m, 1H), 4.30-4.18 (m, 2H), 3.16-3.03 (m, 2H), 2.98-2.87 (m, 2H), 2.69-2.64 (m, 8H), 2.40 (s, 3H), 2.37-2.29 (m, 1H), 1.78-1.65 (m, 1H), 1.27 (d, J=6.9 Hz, 3H).

### Example 99

MS: 476 [M+H]⁺. ¹H NMR (400 MHz, Methanol-d₄, ppm) δ 8.97-8.91 (m, 2H), 8.14 (d, J=8.2 Hz, 1H), 7.35 (d, J=8.4 Hz, 1H), 4.69-4.61 (m, 1H), 4.26-4.19 (m, 2H), 3.15-3.03 (m, 2H), 2.98-2.87 (m, 2H), 2.80-2.52 (m, 8H), 2.42 (s, 3H), 2.36-2.29 (m, 1H), 1.79-1.66 (m, 1H), 1.28 (d, J=6.9 Hz, 3H).

### Example 100: (2R)—N-[(3R,5S)-1-(8-cyanoquinolin-5-yl)-5-(trifluoromethyl)piperidin-3-yl]-2-(4-methylpiperazin-1-yl)propanamide & Example 101: (2S)—N-[(3R,5S)-1-(8-cyanoquinolin-5-yl)-5-(trifluoromethyl)piperidin-3-yl]-2-(4-methylpiperazin-1-yl)propenamide

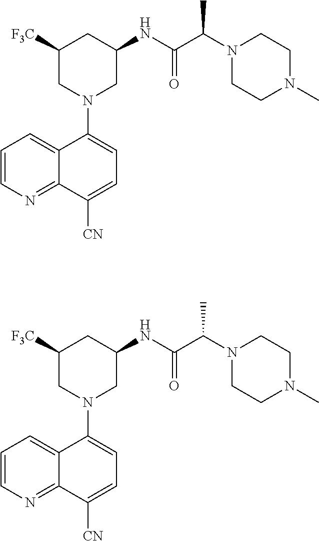 [SMILES: C[C@H](C(=O)N[C@@H]1C[C@H](C(F)(F)F)CN(c2ccc(C#N)c3ncccc23)C1)N1CCN(C)CC1 ; C[C@@H](C(=O)N[C@@H]1C[C@H](C(F)(F)F)CN(c2ccc(C#N)c3ncccc23)C1)N1CCN(C)CC1]

The title compounds were prepared from 5-[(3R,5S)-3-amino-5-(trifluoromethyl)piperidin-1-yl]quinoline-8-carbonitrile and 2-(4-methylpiperazin-1-yl)propanoic acid, followed by separation on chiral-HPLC under the following conditions: column Repaired ADH, 0.46×10 cm, 3 um; mobile phase, hexane (with 0.1% DEA) in EtOH, 90% isocratic in 20 min; detector, UV 220 nm. (chirality of 2-(4-methylpiperazin-1-yl)propanamide was assigned arbitrarily).

### Example 100

MS: 475 [M+H]⁺. ¹H NMR (400 MHz, Methanol-d₄, ppm) δ 9.04-8.98 (m, 1H), 8.68 (dd, J=8.6, 1.7 Hz, 1H), 8.16 (d, J=8.0 Hz, 1H), 7.70 (dd, J=8.6, 4.3 Hz, 1H), 7.33 (d, J=8.0 Hz, 1H), 4.39-4.27 (m, 1H), 3.74-3.63 (m, 2H), 3.11-2.95 (m, 3H), 2.73-2.41 (m, 9H), 2.38-2.31 (m, 1H), 2.30 (s, 3H), 1.74-1.61 (m, 1H), 1.25 (d, J=6.9 Hz, 3H).

### Example 101

MS: 475 [M+H]⁺. ¹H NMR (400 MHz, Methanol-d₄, ppm) δ 9.04-8.98 (m, 1H), 8.68 (dd, J=8.6, 1.7 Hz, 1H), 8.16 (d, J=8.0 Hz, 1H), 7.70 (dd, J=8.6, 4.3 Hz, 1H), 7.33 (d, J=8.0 Hz, 1H), 4.39-4.27 (m, 1H), 3.74-3.63 (m, 2H), 3.11-2.95 (m, 3H), 2.73-2.41 (m, 9H), 2.38-2.31 (m, 1H), 2.30 (s, 3H), 1.74-1.61 (m, 1H), 1.25 (d, J=6.9 Hz, 3H).

### Example 102: (2S)—N-[(3R,5S)-1-(8-cyanoquinoxalin-5-yl)-5-methylpiperidin-3-yl]-2-(4-hydroxypiperidin-1-yl)propanamide & Example 103: (2R)—N-[(3R,5S)-1-(8-cyanoquinoxalin-5-yl)-5-methylpiperidin-3-yl]-2-(4-hydroxypiperidin-1-yl)propanamide

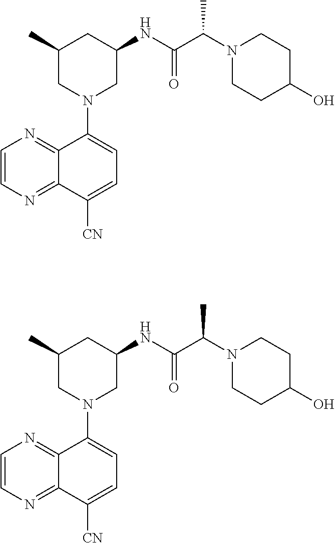 [SMILES: C[C@H]1C[C@@H](NC(=O)[C@H](C)N2CCC(O)CC2)CN(c2ccc(C#N)c3nccnc23)C1 ; C[C@H]1C[C@@H](NC(=O)[C@@H](C)N2CCC(O)CC2)CN(c2ccc(C#N)c3nccnc23)C1]

The title compounds were prepared from 8-[(3R,5S)-3-amino-5-methylpiperidin-1-yl]quinoxaline-5-carbonitrile and 2-(4-hydroxypiperidin-1-yl)propanoic acid, followed by separation on chiral-HPLC under the following conditions: column, CHIRALPAK IE-3, 0.46×10 cm, 3 um; mobile phase, hexane (with 0.1% DEA) in EtOH, 90% isocratic in 20 min; detector, UV 254 nm. (chirality of 2-(4-hydroxypiperidin-1-yl)propenamide was assigned arbitrarily).

### Example 102

MS: 423 [M+H]⁺. ¹H NMR (400 MHz, Methanol-d₄, ppm) δ 8.93-8.88 (m, 2H), 8.10 (d, J=8.4 Hz, 1H), 7.29 (d, J=8.4 Hz, 1H), 4.86 (br s, 1H), 4.41-4.23 (m, 2H), 4.18-4.13 (m, 1H), 3.66-3.57 (m, 1H), 3.14-3.05 (m, 1H), 2.89-2.79 (m, 2H), 2.71 (t, J=11.7 Hz, 1H), 2.45-2.35 (m, 1H), 2.33-2.23 (m, 1H), 2.14-2.03 (m, 3H), 1.91-1.85 (m, 2H), 1.65-1.51 (m, 2H), 1.37-1.21 (m, 4H), 1.03 (d, J=6.4 Hz, 3H).

### Example 103

MS: 423 [M+H]⁺. ¹H NMR (400 MHz, Methanol-d₄, ppm) δ 8.94-8.87 (m, 2H), 8.10 (d, J=8.4 Hz, 1H), 7.30 (d, J=8.4 Hz, 1H), 4.60 (s, 1H), 4.32 (dd, J=22.5, 12.3 Hz, 2H), 4.14 (m, J=8.4, 5.7, 4.1 Hz, 1H), 3.62 (m, J=9.2, 4.8 Hz, 1H), 3.05 (t, J=6.9 Hz, 1H), 2.87-2.78 (m, 2H), 2.78-2.69 (m, 1H), 2.37 (t, J=10.6 Hz, 1H), 2.27 (t, J=10.7 Hz, 1H), 2.15-2.05 (m, 2H), 1.96-1.77 (m, 2H), 1.59 (t, J=11.0 Hz, 2H), 1.33-1.28 (m, 1H), 1.24 (s, 3H), 1.03 (d, J=6.4 Hz, 3H).

### Example 104: (2S)-2-(4-hydroxypiperidin-1-yl)-N-[(3R,5S)-5-methyl-1-[8-(trifluoromethyl)quinolin-5-yl]piperidin-3-yl]propanamide & Example 105: (2R)-2-(4-hydroxypiperidin-1-yl)-N-[(3R,5S)-5-methyl-1-[8-(trifluoromethyl)quinolin-5-yl]piperidin-3-yl]propanamide

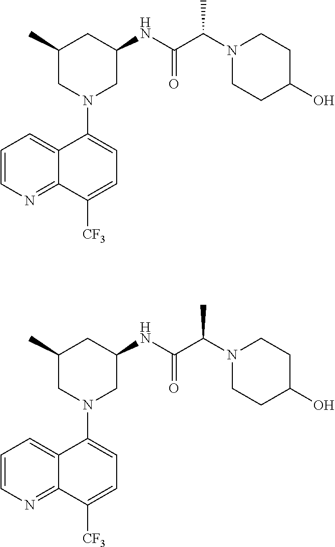 [SMILES: C[C@H]1C[C@@H](NC(=O)[C@H](C)N2CCC(O)CC2)CN(c2ccc(C(F)(F)F)c3ncccc23)C1 ; C[C@H]1C[C@@H](NC(=O)[C@@H](C)N2CCC(O)CC2)CN(c2ccc(C(F)(F)F)c3ncccc23)C1]

The title compounds were prepared from (3R,5S)-5-methyl-1-[8-(trifluoromethyl)quinolin-5-yl]piperidin-3-amine and 2-(4-hydroxypiperidin-1-yl)propanoic acid, followed by separation on chiral-HPLC under the following conditions: column, CHIRALPAK IE-3, 0.46×5 cm, 3 um; mobile phase, hexane (20 mmol NH₃) in EtOH, 85% isocratic in 20 min; detector, UV 254 nm. (chirality of 2-(4-hydroxypiperidin-1-yl)propenamide was assigned arbitrarily).

### Example 104

MS: 465 [M+H]⁺. ¹H NMR (400 MHz, Methanol-d₄, ppm) δ 8.94 (dd, J=4.2, 1.8 Hz, 1H), 8.66 (dd, J=8.6, 1.8 Hz, 1H), 8.03 (d, J=8.0 Hz, 1H), 7.63 (dd, J=8.6, 4.2 Hz, 1H), 7.23 (d, J=8.0 Hz, 1H), 4.31-4.18 (m, 1H), 3.68-3.56 (m, 2H), 3.43-3.35 (m, 1H), 3.13-3.04 (m, 1H), 2.88-2.76 (m, 2H), 2.60-2.45 (m, 2H), 2.46-2.24 (m, 2H), 2.22-2.09 (m, 2H), 1.91-1.87 (m, 2H), 1.65-1.52 (m, 2H), 1.26-1.14 (m, 4H), 1.05 (d, J=6.5 Hz, 3H).

### Example 105

MS: 465 [M+H]⁺. ¹H NMR (400 MHz, Methanol-d₄, ppm) δ 8.94 (dd, J=4.2, 1.8 Hz, 1H), 8.66 (dd, J=8.6, 1.8 Hz, 1H), 8.03 (d, J=8.1 Hz, 1H), 7.63 (dd, J=8.6, 4.2 Hz, 1H), 7.23 (d, J=8.0 Hz, 1H), 4.31-4.18 (m, 1H), 3.62 (m, J=12.8, 11.2, 4.4 Hz, 2H), 3.43-3.35 (m, 1H), 3.09 (m, J=6.9 Hz, 1H), 2.82 (m, J=12.2, 5.7 Hz, 2H), 2.53 (m, J=22.2, 11.1 Hz, 2H), 2.35 (m, J=46.2, 10.7 Hz, 2H), 2.21-2.09 (m, 2H), 1.87 (d, J=13.3 Hz, 2H), 1.59 (m, J=12.6, 8.0, 3.3 Hz, 2H), 1.27-1.14 (m, 4H), 1.05 (d, J=6.5 Hz, 3H).

### Example 106: N-[(3R,5S)-5-methyl-1-[8-(trifluoromethyl)quinolin-5-yl]piperidin-3-yl]-2-[(1R,5S,6s)-3-methyl-3-azabicyclo[3.1.1]heptan-6-yl]acetamide & Example 107: N-((3S,5R)-5-methyl-1-(8-(trifluoromethyl)quinolin-5-yl)piperidin-3-yl)-2-((1R,5S,6r)-3-methyl-3-azabicyclo[3.1.1]heptan-6-yl)acetamide

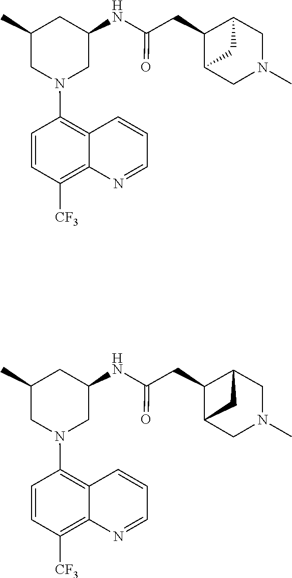

The title compounds were prepared from (3R,5S)-5-methyl-1-[8-(trifluoromethyl)quinolin-5-yl]piperidin-3-amine and 2-(4-hydroxypiperidin-1-yl)propanoic acid, followed by separation on prep-HPLC under the following conditions: column, XBridge Prep C18 OBD Column, 150×19 mm, 5 um; mobile phase, acetonitrile in water (with 10 mmol/L NH₄HCO₃ and 0.1% NH₃.H₂O), 32% to 68% gradient in 8 min; detector, UV 254 nm.

### Example 106

MS: 461 [M+H]⁺. ¹H NMR (300 MHz, DMSO-d₆, ppm) δ 9.01 (dd, J=4.2, 1.7 Hz, 1H), 8.52 (dd, J=8.6, 1.8 Hz, 1H), 8.05 (d, J=8.1 Hz, 1H), 7.88 (d, J=7.4 Hz, 1H), 7.66 (dd, J=8.6, 4.2 Hz, 1H), 7.19 (d, J=8.1 Hz, 1H), 4.06-4.0 (m, 1H), 3.54-3.45 (m, 1H), 3.37-3.32 (m, 1H), 2.99-2.83 (m, 2H), 2.71-2.58 (m, 2H), 2.48-2.22 (m, 6H), 2.18-2.12 (m, 1H), 2.05-1.93 (m, 5H), 1.54-1.48 (m, 1H), 1.27-1.21 (m, 1H), 1.12-1.05 (m, 1H), 0.94 (d, J=6.4 Hz, 3H).

### Example 107

MS: 461 [M+H]⁺. ¹H NMR (300 MHz, DMSO-d₆, ppm) δ 8.95 (dd, J=4.3, 1.7 Hz, 1H), 8.66 (dd, J=8.6, 1.8 Hz, 1H), 8.05 (d, J=8.0 Hz, 1H), 7.64 (dd, J=8.6, 4.2 Hz, 1H), 7.24 (d, J=8.0 Hz, 1H), 4.23 (s, 1H), 3.64 (d, J=11.4 Hz, 2H), 3.45 (m, 1H), 3.16 (d, J=11.6 Hz, 2H), 2.67 (d, J=25.2 Hz, 4H), 2.58-2.31 (m, 6H), 2.16 (s, 3H), 1.81 (d, J=9.9 Hz, 1H), 1.39-1.02 (m, 5H).

### Example 108: 2-(1-Isopropyl-piperidin-4-yl)-N-[(3R,5S)-5-methyl-1-(8-methyl-quinolin-5-yl)-piperidin-3-yl]-acetamide

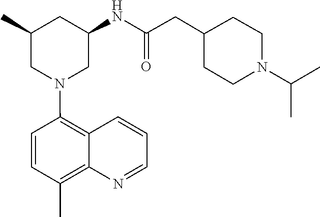 [SMILES: Cc1ccc(N2C[C@@H](C)C[C@@H](NC(=O)CC3CCN(C(C)C)CC3)C2)c2cccnc12]

The title compound was synthesized from (3R,5S)-5-Methyl-1-(8-methyl-quinolin-5-yl)-piperidin-3-ylamine hydrochloride (2) and (1-Isopropyl-piperidin-4-yl)-acetic acid. MS: 423.6 [M+H]⁺. H NMR (400 MHz, DMSO-d₆) δ 8.91 (dd, J=4.1, 1.8 Hz, 1H), 8.47 (dd, J=8.5, 1.8 Hz, 1H), 7.79 (d, J=7.6 Hz, 1H), 7.55 (dd, J=8.5, 4.1 Hz, 1H), 7.51 (dd, J=7.6, 1.1 Hz, 1H), 7.08 (d, J=7.6 Hz, 1H), 4.10-3.98 (m, 1H), 3.16 (d, J=10.6 Hz, 1H), 2.75-2.65 (m, 3H), 2.65 (d, J=1.0 Hz, 3H), 2.64-2.58 (m, 1H), 2.32 (dt, J=18.3, 10.9 Hz, 2H), 2.08-1.99 (m, 3H), 1.97 (d, J=6.8 Hz, 3H), 1.56 (t, J=12.8 Hz, 3H), 1.06 (dq, J=24.1, 11.9 Hz, 4H), 0.93 (dd, J=6.5, 3.5 Hz, 9H).

### Example 109: 2-(1-Isopropyl-piperidin-4-yl)-N-[(3R,5S)-1-(8-methyl-quinolin-5-yl)-5-trifluoromethyl-piperidin-3-yl]-acetamide

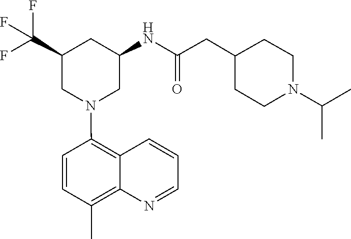 [SMILES: Cc1ccc(N2C[C@H](NC(=O)CC3CCN(C(C)C)CC3)C[C@H](C(F)(F)F)C2)c2cccnc12]

The title compound was synthesized from (3R,5S)-1-(8-Methyl-quinolin-5-yl)-5-trifluoromethyl-piperidin-3-ylamine hydrochloride (2) and (1-Isopropyl-piperidin-4-yl)-acetic acid. MS: 477.6. [M+H]⁺. 1H NMR (400 MHz, DMSO-d₆) δ 8.93 (dd, J=4.1, 1.8 Hz, 1H), 8.53 (dd, J=8.5, 1.8 Hz, 1H), 7.93 (d, J=7.5 Hz, 1H), 7.58 (dd, J=8.5, 4.1 Hz, 1H), 7.54 (dd, J=7.5, 1.1 Hz, 1H), 7.19 (d, J=7.6 Hz, 1H), 4.13 (s, 1H), 3.14 (s, 1H), 2.76 (t, J=11.4 Hz, 1H), 2.70 (s, 2H), 2.61 (q, J=6.6 Hz, 1H), 2.16 (d, J=12.2 Hz, 1H), 2.08-1.99 (m, 2H), 1.97 (s, 2H), 1.57 (t, J=12.5 Hz, 3H), 1.44 (q, J=12.3 Hz, 1H), 1.08 (s, 2H), 0.93 (d, J=6.6 Hz, 6H).

### Example 110: N—[(R)-5,5-Difluoro-1-(8-methyl-quinolin-5-yl)-piperidin-3-yl]-2-(1-isopropyl-piperidin-4-yl)-acetamide & Example 111: N—[(S)-5,5-Difluoro-1-(8-methyl-quinolin-5-yl)-piperidin-3-yl]-2-(1-isopropyl-piperidin-4-yl)-acetamide & Example

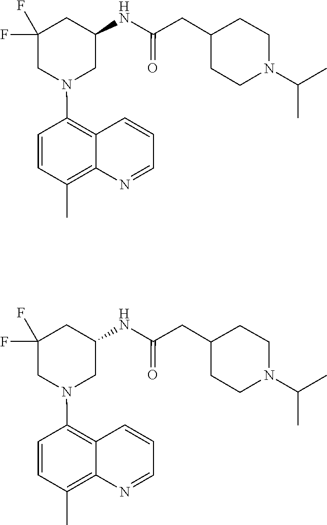 [SMILES: Cc1ccc(N2C[C@H](NC(=O)CC3CCN(C(C)C)CC3)CC(F)(F)C2)c2cccnc12 ; Cc1ccc(N2C[C@@H](NC(=O)CC3CCN(C(C)C)CC3)CC(F)(F)C2)c2cccnc12]

The title compound was synthesized from 5,5-Difluoro-1-(8-methyl-quinolin-5-yl)-piperidin-3-ylamine hydrochloride and (1-Isopropyl-piperidin-4-yl)-acetic acid, followed by chiral SFC separation under the following conditions: column, IA, Prep SFC-P100; mobile phase, 0.5% dimethylethylamine (DMEA) in ethanol, 40° C./80 bar, 70 g/min; wavelength: 240 nm.

### Example 110

MS: 44.6 [M+H]⁺. 1H NMR (400 MHz, DMSO-d₆) δ 8.94 (dd, J=4.1, 1.8 Hz, 1H), 8.51 (dd, J=8.5, 1.8 Hz, 1H), 7.97 (d, J=7.5 Hz, 1H), 7.59 (dd, J=8.5, 4.1 Hz, 1H), 7.55 (dd, J=7.6, 1.1 Hz, 1H), 7.20 (d, J=7.6 Hz, 1H), 4.24 (d, J=5.7 Hz, 0H), 2.70 (d, J=9.9 Hz, 2H), 2.67 (d, J=0.9 Hz, 3H), 2.61 (p, J=6.7 Hz, 1H), 2.44 (d, J=11.5 Hz, 1H), 2.06-1.95 (m, 5H), 1.57 (s, 3H), 1.16-1.02 (m, 2H), 0.92 (d, J=6.6 Hz, 6H).

### Example 111

MS: 445.6 [M+H]⁺. 1H NMR (400 MHz, DMSO-d₆) δ 8.94 (dd, J=4.1, 1.8 Hz, 1H), 8.51 (dd, J=8.5, 1.8 Hz, 1H), 7.97 (d, J=7.5 Hz, 1H), 7.59 (dd, J=8.5, 4.1 Hz, 1H), 7.55 (dd, J=7.6, 1.1 Hz, 1H), 7.20 (d, J=7.6 Hz, 1H), 4.23 (d, J=5.7 Hz, 0H), 2.70 (d, J=9.9 Hz, 2H), 2.67 (d, J=0.9 Hz, 3H), 2.61 (p, J=6.7 Hz, 1H), 2.44 (d, J=11.5 Hz, 1H), 2.06-1.95 (m, 5H), 1.56 (s, 3H), 1.16-1.02 (m, 2H), 0.92 (d, J=6.6 Hz, 6H).

### Example 112: N-[(3R,5S)-1-(7-Fluoro-8-methyl-quinolin-5-yl)-5-methyl-piperidin-3-yl]-2-(3-methyl-3-aza-bicyclo[3.1.1]hept-6-yl)-acetamide

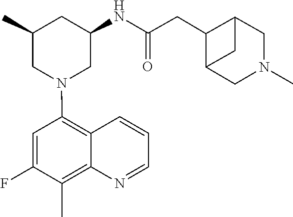

The title compound was synthesized from (3R,5S)-1-(7-Fluoro-8-methyl-quinolin-5-yl)-5-methyl-piperidin-3-ylamine hydrochloride and (3-Methyl-3-aza-bicyclo[3.1.1]hept-6-yl)-acetic acid. MS: 425.6 [M+H]⁺. 1H NMR (400 MHz, DMSO-d₆) δ 8.93 (dd, J=4.2, 1.7 Hz, 1H), 8.44 (dt, J=8.4, 1.6 Hz, 1H), 7.81 (dd, J=23.9, 7.5 Hz, 1H), 7.52 (dd, J=8.5, 4.2 Hz, 1H), 7.04 (d, J=11.5 Hz, 1H), 4.08-3.93 (m, 1H), 3.20 (d, J=11.4 Hz, 1H), 2.90-2.71 (m, 4H), 2.64-2.58 (m, 1H), 2.53 (d, J=2.3 Hz, 3H), 2.41-2.31 (m, 3H), 2.29 (d, J=6.4 Hz, 3H), 2.25-2.12 (m, 3H), 2.04-1.92 (m, 3H), 1.57-1.48 (m, 1H), 1.05 (q, J=12.0 Hz, 1H), 0.94 (d, J=6.5 Hz, 3H).

### Example 113: N-[(3R,5S)-1-(7-Fluoro-8-methyl-quinolin-5-yl)-5-trifluoromethyl-piperidin-3-yl]-2-(3-methyl-3-aza-bicyclo[3.1.1]hept-6-yl)-acetamide

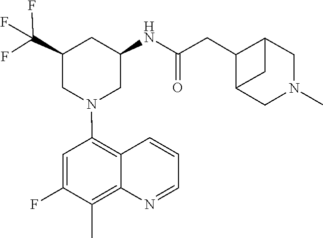 [SMILES: Cc1c(F)cc(N2C[C@H](NC(=O)CC3C4CC3CN(C)C4)C[C@H](C(F)(F)F)C2)c2cccnc12]

The title compound was synthesized from (3R,5S)-1-(7-Fluoro-8-methyl-quinolin-5-yl)-5-trifluoromethyl-piperidin-3-ylamine and (3-Methyl-3-aza-bicyclo[3.1.1]hept-6-yl)-acetic acid. MS: 479.5 [M+H]⁺. 1H NMR (400 MHz, DMSO-d₆) δ 8.95 (dd, J=4.2, 1.7 Hz, 1H), 8.49 (dd, J=8.5, 1.7 Hz, 1H), 7.92 (d, J=7.5 Hz, 1H), 7.55 (dd, J=8.5, 4.2 Hz, 1H), 7.20 (d, J=11.3 Hz, 1H), 4.11 (dd, J=10.8, 5.0 Hz, 2H), 3.14 (s, 1H), 2.90-2.70 (m, 5H), 2.62 (d, J=13.4 Hz, 1H), 2.55 (d, J=2.4 Hz, 3H), 2.48-2.34 (m, 2H), 2.29 (s, 2H), 2.25-2.09 (m, 4H), 1.77-1.70 (m, 1H), 1.53 (d, J=8.3 Hz, 1H), 1.45 (q, J=12.3 Hz, 1H).

### Example 114: N-[(3R,5S)-1-(8-cyanoquinoxalin-5-yl)-5-methylpiperidin-3-yl]-2-(4-fluoropiperidin-4-yl)acetamide

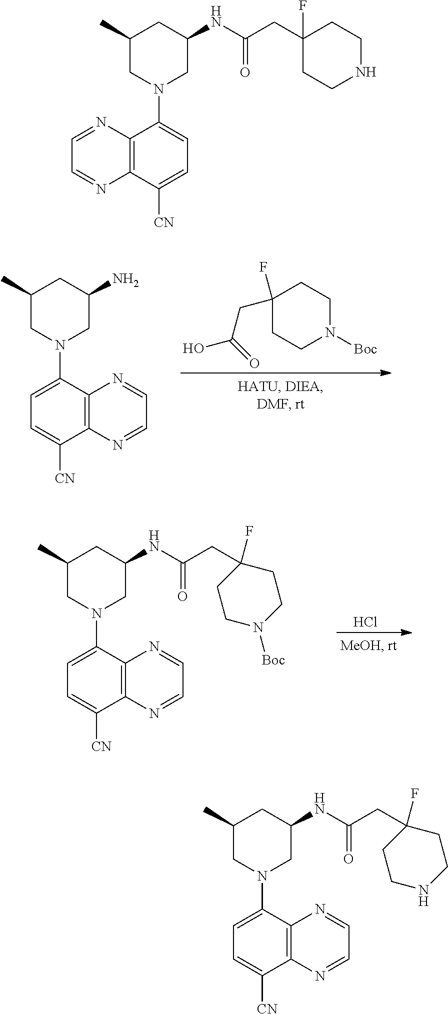 [SMILES: C[C@H]1C[C@@H](NC(=O)CC2(F)CCNCC2)CN(c2ccc(C#N)c3nccnc23)C1 ; C[C@H]1C[C@@H](N)CN(c2ccc(C#N)c3nccnc23)C1 ; CC(C)(C)OC(=O)N1CCC(F)(CC(=O)O)CC1 ; C[C@H]1C[C@@H](NC(=O)CC2(F)CCN(C(=O)OC(C)(C)C)CC2)CN(c2ccc(C#N)c3nccnc23)C1 ; Cl ; C[C@H]1C[C@@H](NC(=O)CC2(F)CCNCC2)CN(c2ccc(C#N)c3nccnc23)C1]

tert-butyl 4-([[(3R)-1-(8-cyanoquinoxalin-5-yl)-5-methyl-1,2,3,6-tetrahydropyridin-3-yl]carbamoyl]methyl)-4-fluoropiperidine-1-carboxylate: To a solution of 8-[(3R)-3-amino-5-methyl-1,2,3,6-tetrahydropyridin-1-yl]quinoxaline-5-carbonitrile (61 mg, 0.23 mmol) in DMF (3 mL) was added 2-[1-[(tert-butoxy)carbonyl]-4-fluoropiperidin-4-yl]acetic acid (211 mg, 0.81 mmol), DIEA (184 mg, 1.43 mmol), HATU (361 mg, 0.95 mmol) at room temperature. The resulting mixture was stirred for 3 h at room temperature. When the reaction was done, it was quenched by the addition of water (10 mL). The resulting mixture was extracted with ethyl acetate (30 mL×3). The organic phases were combined, washed with brine and dried over Na₂SO₄. The solvent was removed under reduced pressure to yield N-[(3R,5S)-1-(8-cyanoquinoxalin-5-yl)-5-methylpiperidin-3-yl]-2-(4-fluoropiperidin-4-yl)acetamide as yellow solid (60 mg, crude), which was used in next step without further purification.

N-[(3R,5S)-1-(8-cyanoquinoxalin-5-yl)-5-methylpiperidin-3-yl]-2-(4-fluoropiperidin-4-yl)acetamide: To a solution of tert-butyl 4-([[(3R,5S)-1-(8-cyanoquinoxalin-5-yl)-5-methylpiperidin-3-yl]carbamoyl]methyl)-4-fluoropiperidine-1-carboxylate (60 mg, crude) in methanol (3 mL) was added hydrogen chloride aqueous solution (6 N, 1 mL, 6.0 mmol) at room temperature. The resulting mixture was stirred for 3 h at room temperature. When the reaction was done, it was quenched by the addition of water (10 mL). The resulting mixture was extracted with ethyl acetate (30 mL×3). The organic phases were combined, washed with brine and dried over Na₂SO₄. The solvent was removed under reduced pressure and the residue was purified by prep-HPLC under the following conditions: column, XBridge Prep C18 OBD, 150×19 mm, 5 um; mobile phase, acetonitrile in water (with 10 mmol/L NH₄HCO₃ and 0.1% NH₃.H₂O), 15% to 45% gradient in 8 min; detector, UV 254 nm. N-[(3R,5S)-1-(8-cyanoquinoxalin-5-yl)-5-methylpiperidin-3-yl]-2-(4-fluoropiperidin-4-yl)acetamide was obtained as a yellow solid (26 mg, 30% for 2 steps). MS: 411 [M+H]⁺. ¹H NMR (400 MHz, Methanol-d₄, ppm) δ 8.93 (d, J=1.8 Hz, 1H), 8.89 (d, J=1.8 Hz, 1H), 8.10 (d, J=8.4 Hz, 1H), 7.29 (d, J=8.4 Hz, 1H), 4.44-4.27 (m, 2H), 4.17-4.12 (m, 1H), 2.93-2.86 (m, 4H), 2.78 (t, J=11.3 Hz, 1H), 2.68 (t, J=11.6 Hz, 1H), 2.53 (d, J=16.0 Hz, 2H), 2.17-1.67 (m, 6H), 1.33-1.16 (m, 1H), 1.02 (d, J=6.5 Hz, 3H).

The following compound was synthesized in an analogous manner.

### Example 115: (2S)—N-[(3R,5S)-1-(7-fluoro-8-methylquinolin-5-yl)-5-(trifluoromethyl)piperidin-3-yl]pyrrolidine-2-carboxamide

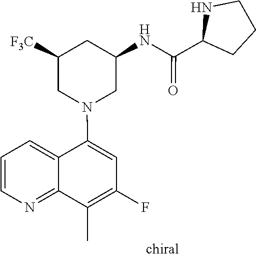 [SMILES: Cc1c(F)cc(N2C[C@H](NC(=O)[C@@H]3CCCN3)C[C@H](C(F)(F)F)C2)c2cccnc12]

The title compound was prepared from (3R,5S)-1-(7-fluoro-8-methylquinolin-5-yl)-(trifluoromethyl)piperidin-3-amine and (2S)-1-[(tert-butoxy)carbonyl]pyrrolidine-2-carboxylic acid. MS: 425 [M+H]⁺. ¹H NMR (400 MHz, DMSO-d₆, ppm) δ 8.98-8.93 (m, 1H), 8.50 (dd, J=8.4, 1.8 Hz, 1H), 8.04 (d, J=8.1 Hz, 1H), 7.55 (dd, J=8.5, 4.2 Hz, 1H), 7.21 (d, J=11.3 Hz, 1H), 4.17-4.12 (m, 1H), 3.54-3.46 (m, 1H), 3.27-3.06 (m, 3H), 2.87-2.75 (m, 3H), 2.66-2.57 (m, 1H), 2.55 (d, J=2.3 Hz, 3H), 2.17-2.09 (m, 1H), 2.02-1.87 (m, 1H), 1.72-1.51 (m, 4H).

### Example 116: (2S,4S)—N-[(3R,5S)-1-(8-cyanoquinoxalin-5-yl)-5-methylpiperidin-3-yl]-4-hydroxypyrrolidine-2-carboxamide

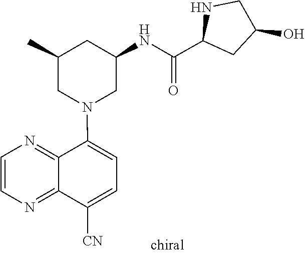 [SMILES: C[C@H]1C[C@@H](NC(=O)[C@@H]2C[C@H](O)CN2)CN(c2ccc(C#N)c3nccnc23)C1]

The title compound was prepared from 8-[(3R,5S)-3-amino-5-methylpiperidin-1-yl]quinoxaline-5-carbonitrile and (2S,4S)-1-[(tert-butoxy)carbonyl]-4-hydroxypyrrolidine-2-carboxylic acid. MS: 381 [M+H]⁺. ¹H NMR (300 MHz, Methanol-d₄, ppm) δ 8.91-8.87 (m, 2H), 8.09 (d, J=8.4 Hz, 1H), 7.28 (d, J=8.5 Hz, 1H), 4.45-4.23 (m, 3H), 4.15-4.09 (m, 1H), 3.71-3.61 (m, 1H), 3.05-2.90 (m, 2H), 2.86-2.57 (m, 2H), 2.40-2.25 (m, 1H), 2.16-2.01 (m, 2H), 1.91-1.81 (m, 1H), 1.37-1.23 (m, 1H), 1.02 (d, J=6.4 Hz, 3H).

### Example 117: 2-(4-fluoropiperidin-4-yl)-N-[(3R,5S)-5-(trifluoromethyl)-1-[8-(trifluoromethyl)quinolin-5-yl]piperidin-3-yl]acetamide

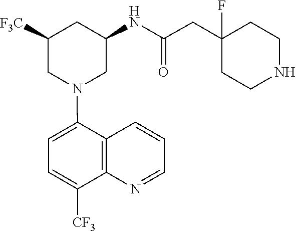 [SMILES: O=C(CC1(F)CCNCC1)N[C@@H]1C[C@H](C(F)(F)F)CN(c2ccc(C(F)(F)F)c3ncccc23)C1]

The title compound was prepared from tert-butyl 4-fluoro-4-({[(3R,5S)-5-(trifluoromethyl)-1-[8-(trifluoromethyl)quinolin-5-yl]piperidin-3-yl]carbamoyl}methyl)piperidine-1-carboxylate and hydrogen chloride in dioxane. MS: 507 [M+H]⁺. ¹H NMR (400 MHz, DMSO-d₆, ppm) δ 9.07-9.01 (m, 1H), 8.60 (dd, J=8.6, 1.8 Hz, 1H), 8.11 (dd, J=16.0, 7.7 Hz, 2H), 7.71 (dd, J=8.6, 4.2 Hz, 1H), 7.32 (d, J=8.1 Hz, 1H), 4.22-4.05 (m, 1H), 3.54-3.47 (m, 2H), 3.24-3.20 (m, 2H), 2.89 (t, J=11.5 Hz, 1H), 2.79-2.55 (m, 5H), 2.43 (d, J=18.7 Hz, 2H), 2.22-2.14 (m, 1H), 1.78-1.45 (m, 5H).

### Example 118: N-[(3R,5S)-1-(8-cyanoquinoxalin-5-yl)-5-(trifluoromethyl)piperidin-3-yl]-2-(4-fluoropiperidin-4-yl)acetamide

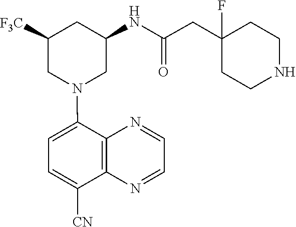 [SMILES: N#Cc1ccc(N2C[C@H](NC(=O)CC3(F)CCNCC3)C[C@H](C(F)(F)F)C2)c2nccnc12]

The title compound was prepared from tert-butyl 4-({[(3R,5S)-1-(8-cyanoquinoxalin-5-yl)-5-(trifluoromethyl)piperidin-3-yl]carbamoyl}methyl)-4-fluoropiperidine-1-carboxylate and hydrogen chloride in dioxane. MS: 465 [M+H]⁺. ¹H NMR (300 MHz, DMSO-d₆, ppm) δ 9.04 (d, J=1.8 Hz, 1H), 8.96 (d, J=1.8 Hz, 1H), 8.24 (d, J=8.4 Hz, 1H), 8.14 (d, J=7.2 Hz, 1H), 7.34 (d, J=8.4 Hz, 1H), 4.71-4.61 (m, 1H), 4.17-3.90 (m, 2H), 3.10 (t, J=11.9 Hz, 1H), 2.99-2.93 (m, 1H), 2.84 (t, J=11.5 Hz, 1H), 2.76-2.59 (m, 4H), 2.51-2.40 (m, 2H), 2.17-2.08 (m, 2H), 1.80-1.45 (m, 5H).

### Example 119: 2-(1-aminocyclopropyl)-N-[(3R,5S)-1-(8-cyanoquinoxalin-5-yl)-5-(trifluoromethyl)piperidin-3-yl]acetamide

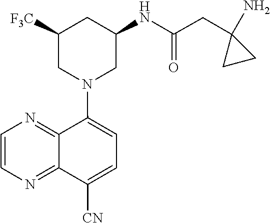 [SMILES: N#Cc1ccc(N2C[C@H](NC(=O)CC3(N)CC3)C[C@H](C(F)(F)F)C2)c2nccnc12]

The title compound was prepared from tert-butyl N-[1-({[(3R,5S)-1-(8-cyanoquinoxalin-5-yl)-5-(trifluoromethyl)piperidin-3-yl]carbamoyl}methyl)cyclopropyl]carbamate and hydrogen chloride in dioxane. MS: 419 [M+H]⁺. ¹H NMR (400 MHz, Methanol-d₄, ppm) δ 9.0-8.91 (m, 2H), 8.14 (d, J=8.4, 1.8 Hz, 1H), 7.35 (d, J=8.4, 1.5 Hz, 1H), 4.67 (d, J=11.7 Hz, 1H), 4.31-4.17 (m, 2H), 3.15-2.78 (m, 3H), 2.37-2.33 (m, 3H), 1.66-1.57 (m, 1H), 0.70-0.63 (m, 2H), 0.63-0.53 (m, 2H).

### Example 120: 2-(1-aminocyclopropyl)-N-[(3R,5S)-1-(8-cyanoquinoxalin-5-yl)-5-methylpiperidin-3-yl]acetamide

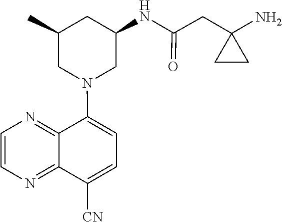 [SMILES: C[C@H]1C[C@@H](NC(=O)CC2(N)CC2)CN(c2ccc(C#N)c3nccnc23)C1]

The title compound was prepared from tert-butyl N-[1-({[(3R,5S)-1-(8-cyanoquinoxalin-5-yl)-5-methylpiperidin-3-yl]carbamoyl}methyl)cyclopropyl]carbamate and hydrogen chloride in dioxane. MS: 365 [M+H]⁺. ¹H NMR (400 MHz, Methanol-d₄, ppm) δ 9.02-8.86 (m, 2H), 8.10 (d, J=8.4 Hz, 1H), 7.30 (d, J=8.4 Hz, 1H), 4.43-4.29 (m, 2H), 4.23-4.11 (m, 1H), 2.85-2.61 (m, 2H), 2.57 (s, 2H), 2.16-2.03 (m, 2H), 1.26-1.16 (m, 1H), 1.08-0.96 (m, 5H), 0.95-0.82 (m, 2H).

### Example 121: 2-(1-aminocyclopropyl)-N-[(3R,5S)-5-methyl-1-(8-methyl-1,7-naphthyridin-5-yl)piperidin-3-yl]acetamide

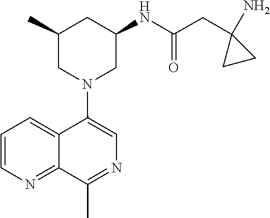 [SMILES: Cc1ncc(N2C[C@@H](C)C[C@@H](NC(=O)CC3(N)CC3)C2)c2cccnc12]

The title compound was prepared from tert-butyl N-[1-({[(3R,5S)-5-methyl-1-(8-methyl-1,7-naphthyridin-5-yl)piperidin-3-yl]carbamoyl}methyl)cyclopropyl]carbamate and hydrogen chloride in dioxane. MS: 354 [M+H]⁺. ¹H NMR (400 MHz, Methanol-d₄, ppm) δ 9.02 (dd, J=4.1, 1.7 Hz, 1H), 8.61 (dd, J=8.6, 1.7 Hz, 1H), 8.05 (s, 1H), 7.79 (dd, J=8.6, 4.2 Hz, 1H), 4.30-4.17 (m, 1H), 3.62-3.54 (m, 1H), 3.33-3.29 (m, 1H), 2.96 (s, 3H), 2.58-2.42 (m, 2H), 2.33 (s, 2H), 2.22-2.07 (m, 2H), 1.25-1.11 (m, 1H), 1.05 (d, J=6.4 Hz, 3H), 0.66-0.61 (m, 2H), 0.59-0.49 (m, 2H).

### Example 122: (2S)—N-[(3R,5S)-5-methyl-1-(8-methylquinolin-5-yl)piperidin-3-yl]pyrrolidine-2-carboxamide

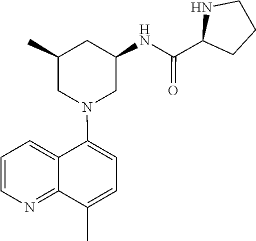 [SMILES: Cc1ccc(N2C[C@@H](C)C[C@@H](NC(=O)[C@@H]3CCCN3)C2)c2cccnc12]

The title compound was prepared from tert-butyl (2S)-2-{[(3R,5S)-5-methyl-1-(8-methylquinolin-5-yl)piperidin-3-yl]carbamoyl}pyrrolidine-1-carboxylateand and hydrogen chloride in dioxane. MS: 353 [M+H]⁺. ¹H NMR (400 MHz, DMSO-d₆, ppm) δ 8.91 (dd, J=4.1, 1.8 Hz, 1H), 8.47 (dd, J=8.5, 1.8 Hz, 1H), 7.84 (d, J=8.1 Hz, 1H), 7.59-7.48 (m, 2H), 7.09 (d, J=7.6 Hz, 1H), 4.03-3.98 (m, 1H), 3.51-3.43 (m, 1H), 3.30-3.23 (m, 1H), 3.19-3.12 (m, 1H), 2.85-2.72 (m, 3H), 2.64 (s, 3H), 2.44 (t, J=10.7 Hz, 1H), 2.30 (t, J=11.2 Hz, 1H), 2.12-1.86 (m, 2H), 1.68-1.50 (m, 3H), 1.18-1.05 (m, 1H), 0.94 (d, J=6.5 Hz, 3H).

### Example 123: (2S)—N-[(3R,5S)-5-(trifluoromethyl)-1-[8-(trifluoromethyl)quinolin-5-yl]piperidin-3-yl]pyrrolidine-2-carboxamide

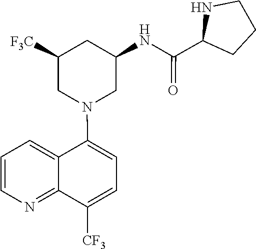 [SMILES: O=C(N[C@@H]1C[C@H](C(F)(F)F)CN(c2ccc(C(F)(F)F)c3ncccc23)C1)[C@@H]1CCCN1]

The title compound was prepared from tert-butyl (2S)-2-{[(3R,5S)-5-(trifluoromethyl)-1-[8-(trifluoromethyl)quinolin-5-yl]piperidin-3-yl]carbamoyl}pyrrolidine-1-carboxylate and hydrogen chloride in dioxane. MS: 461 [M+H]⁺. ¹H NMR (400 MHz, Methanol-d₄, ppm) δ 9.0-8.94 (m, 1H), 8.69 (dd, J=8.6, 1.8 Hz, 1H), 8.08 (d, J=8.0 Hz, 1H), 7.67 (dd, J=8.6, 4.2 Hz, 1H), 7.32 (d, J=8.0 Hz, 1H), 4.37-4.26 (m, 1H), 3.67-3.57 (m, 3H), 3.19-2.80 (m, 4H), 2.65 (t, J=11.1 Hz, 1H), 2.40-2.32 (m, 1H), 2.21-2.09 (m, 1H), 1.85-1.70 (m, 3H), 1.70-1.56 (m, 1H).

### Example 124: (2S)—N-[(3R,5S)-1-(8-cyanoquinoxalin-5-yl)-5-(trifluoromethyl)piperidin-3-yl]pyrrolidine-2-carboxamide

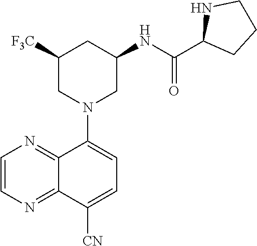 [SMILES: N#Cc1ccc(N2C[C@H](NC(=O)[C@@H]3CCCN3)C[C@H](C(F)(F)F)C2)c2nccnc12]

The title compound was prepared from tert-butyl (2S)-2-{[(3R,5S)-5-(trifluoromethyl)-1-[8-(trifluoromethyl)quinolin-5-yl]piperidin-3-yl]carbamoyl}pyrrolidine-1-carboxylate and hydrogen chloride in dioxane. MS: 419 [M+H]⁺. ¹H NMR (400 MHz, Methanol-d₄, ppm) δ 8.94 (dd, J=16.6, 1.8 Hz, 2H), 8.13 (d, J=8.3 Hz, 1H), 7.33 (d, J=8.4 Hz, 1H), 4.67-4.59 (m, 1H), 4.27-4.15 (m, 2H), 3.71-3.63 (m, 1H), 3.11-2.82 (m, 5H), 2.37-2.30 (m, 1H), 2.25-2.09 (m, 1H), 1.88-1.57 (m, 4H).

### Example 125: (2S)—N-[(3R,5S)-1-(8-cyanoquinolin-5-yl)-5-(trifluoromethyl)piperidin-3-yl]pyrrolidine-2-carboxamide

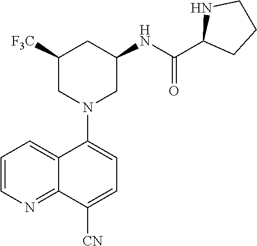 [SMILES: N#Cc1ccc(N2C[C@H](NC(=O)[C@@H]3CCCN3)C[C@H](C(F)(F)F)C2)c2cccnc12]

The title compound was prepared from tert-butyl (2S)-2-{[(3R,5S)-1-(8-cyanoquinolin-5-yl)-5-(trifluoromethyl)piperidin-3-yl]carbamoyl}pyrrolidine-1-carboxylate and hydrogen chloride in dioxane. MS: 418 [M+H]⁺. ¹H NMR (300 MHz, Methanol-d₄, ppm) δ 8.99 (dd, J=4.3, 1.7 Hz, 1H), 8.66 (dd, J=8.6, 1.7 Hz, 1H), 8.15 (d, J=8.0 Hz, 1H), 7.68 (dd, J=8.6, 4.3 Hz, 1H), 7.31 (d, J=8.0 Hz, 1H), 4.43-4.21 (m, 1H), 3.69-3.56 (m, 3H), 3.15-2.82 (m, 4H), 2.65 (t, J=11.2 Hz, 1H), 2.39-2.29 (m, 1H), 2.21-2.06 (m, 1H), 1.87-1.50 (m, 4H).

### Example 126: (2R)—N-[(3R,5S)-1-(8-cyanoquinolin-5-yl)-5-(trifluoromethyl)piperidin-3-yl]pyrrolidine-2-carboxamide

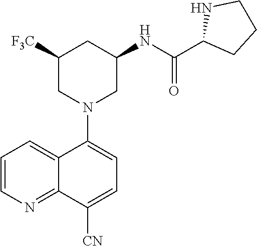 [SMILES: N#Cc1ccc(N2C[C@H](NC(=O)[C@H]3CCCN3)C[C@H](C(F)(F)F)C2)c2cccnc12]

The title compound was prepared from tert-butyl (2R)-2-{[(3R,5S)-1-(8-cyanoquinolin-5-yl)-5-(trifluoromethyl)piperidin-3-yl]carbamoyl}pyrrolidine-1-carboxylate and hydrogen chloride in dioxane. MS: 418 [M+H]⁺. ¹H NMR (400 MHz, Methanol-d₄, ppm) δ 9.01 (dd, J=4.3, 1.7 Hz, 1H), 8.67 (dd, J=8.6, 1.7 Hz, 1H), 8.17 (d, J=8.0 Hz, 1H), 7.69 (dd, J=8.6, 4.2 Hz, 1H), 7.33 (d, J=8.0 Hz, 1H), 4.37-4.25 (m, 1H), 3.74-3.59 (m, 3H), 3.17-2.86 (m, 4H), 2.67 (t, J=11.2 Hz, 1H), 2.39-2.32 (m, 1H), 2.19-2.07 (m, 1H), 1.87-1.50 (m, 4H).

### Example 127: (2S)-2-amino-N-[(3R,5S)-5-methyl-1-[8-(trifluoromethyl)quinoxalin-5-yl]piperidin-3-yl]butanediamide

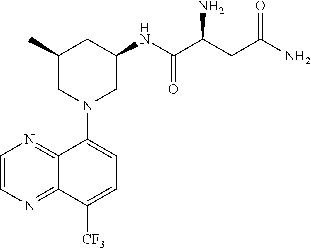 [SMILES: C[C@H]1C[C@@H](NC(=O)[C@@H](N)CC(N)=O)CN(c2ccc(C(F)(F)F)c3nccnc23)C1]

The title compound was prepared from tert-butyl N-[(1S)-2-carbamoyl-1-{[(3R,5S)-5-methyl-1-[8-(trifluoromethyl)quinoxalin-5-yl]piperidin-3-yl]carbamoyl}ethyl]carbamate and hydrogen chloride in dioxane. MS: 425 [M+H]⁺. ¹H NMR (300 MHz, Methanol-d₄, ppm) δ 8.94-8.83 (m, 2H), 8.03 (d, J=8.3 Hz, 1H), 7.28 (d, J=8.3 Hz, 1H), 4.27-3.97 (m, 3H), 3.70-3.59 (m, 1H), 2.77-2.41 (m, 4H), 2.17-2.04 (m, 2H), 1.31-1.15 (m, 1H), 1.01 (d, J=6.4 Hz, 3H).

### Example 128: (2S)-2-amino-N-[(3R,5S)-5-methyl-1-[8-(trifluoromethyl)quinolin-5-yl]piperidin-3-yl]butanediamide

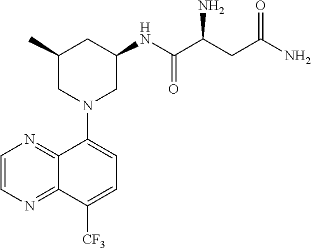 [SMILES: C[C@H]1C[C@@H](NC(=O)[C@@H](N)CC(N)=O)CN(c2ccc(C(F)(F)F)c3nccnc23)C1]

The title compound was prepared from tert-butyl N-[(1S)-2-carbamoyl-1-{[(3R,5S)-5-methyl-1-[8-(trifluoromethyl)quinoxalin-5-yl]piperidin-3-yl]carbamoyl}ethyl]carbamate and hydrogen chloride in dioxane. MS: 424 [M+H]⁺. ¹H NMR (400 MHz, Methanol-d₄, ppm) δ 8.97-8.91 (m, 1H), 8.66 (dd, J=8.6, 1.8 Hz, 1H), 8.04 (d, J=8.1 Hz, 1H), 7.63 (dd, J=8.6, 4.2 Hz, 1H), 7.23 (d, J=8.0 Hz, 1H), 4.25-4.21 (m, 1H), 3.68-3.59 (m, 2H), 3.43-3.36 (m, 1H), 2.67-2.44 (m, 4H), 2.20-2.11 (m, 2H), 1.26-1.13 (m, 1H), 1.05 (d, J=6.4 Hz, 3H).

### Example 129: (2S)-2-amino-N-[(3R,5S)-1-(7-fluoro-8-methylquinolin-5-yl)-5-methylpiperidin-3-yl]butanediamide

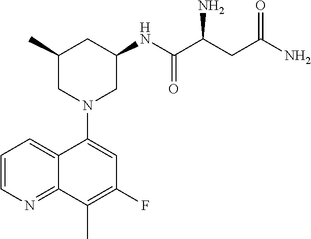 [SMILES: Cc1c(F)cc(N2C[C@@H](C)C[C@@H](NC(=O)[C@@H](N)CC(N)=O)C2)c2cccnc12]

The title compound was prepared from tert-butyl N-[(1S)-2-carbamoyl-1-{[(3R,5S)-1-(7-fluoro-8-methylquinolin-5-yl)-5-methylpiperidin-3-yl]carbamoyl}ethyl]carbamate and hydrogen chloride in dioxane. MS: 388 [M+H]⁺. ¹H NMR (300 MHz, DMSO-d₆, ppm) δ 8.95-8.87 (m, 1H), 8.46-8.37 (m, 1H), 7.82 (d, J=7.8 Hz, 1H), 7.50 (dd, J=8.5, 4.2 Hz, 1H), 7.35 (br s, 1H), 7.01 (d, J=11.6 Hz, 1H), 6.81 (br s, 1H), 4.03-3.94 (m, 1H), 3.47-3.37 (m, 2H), 3.23-3.13 (m, 1H), 2.54-2.47 (m, 3H), 2.46-2.24 (m, 3H), 2.21-1.89 (m, 3H), 1.83 (br s, 2H), 1.17-0.99 (m, 1H), 0.91 (d, J=6.4 Hz, 3H).

### Example 130: (2S)-2-amino-N-[(3R,5S)-5-methyl-1-(8-methylquinolin-5-yl)piperidin-3-yl]butanediamide

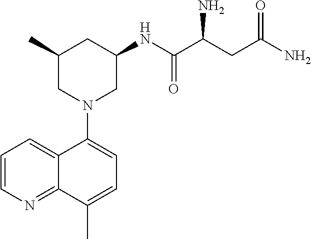 [SMILES: Cc1ccc(N2C[C@@H](C)C[C@@H](NC(=O)[C@@H](N)CC(N)=O)C2)c2cccnc12]

The title compound was prepared from tert-butyl N-[(1S)-2-carbamoyl-1-{[(3R,5S)-5-methyl-1-(8-methylquinolin-5-yl)piperidin-3-yl]carbamoyl}ethyl]carbamate and hydrogen chloride in dioxane. MS: 370 [M+H]⁺. ¹H NMR (400 MHz, DMSO-d₆, ppm) δ 8.93-8.87 (m, 1H), 8.47 (dd, J=8.5, 1.8 Hz, 1H), 7.83 (d, J=7.9 Hz, 1H), 7.58-7.47 (m, 2H), 7.36 (br s, 1H), 7.07 (d, J=7.6 Hz, 1H), 6.83 (br s, 1H), 4.06-3.94 (m, 1H), 3.48-3.41 (m, 3H), 3.20-3.11 (m, 1H), 2.64 (s, 3H), 2.45-2.25 (m, 4H), 2.21-1.88 (m, 3H), 1.15-1.02 (m, 1H), 0.93 (d, J=6.5 Hz, 3H).

### Example 131: N-[(3R,5S)-1-(8-cyanoquinazolin-5-yl)-5-methylpiperidin-3-yl]-2-[(3R,4S)-3-fluoropiperidin-4-yl]acetamide & Example 132: N-[(3R,5S)-1-(8-cyanoquinazolin-5-yl)-5-methylpiperidin-3-yl]-2-[(3S,4R)-3-fluoropiperidin-4-yl]acetamide

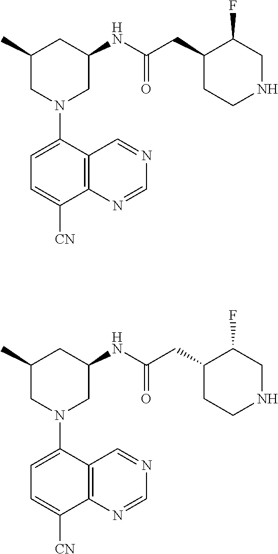 [SMILES: C[C@H]1C[C@@H](NC(=O)C[C@@H]2CCNC[C@@H]2F)CN(c2ccc(C#N)c3ncncc23)C1 ; C[C@H]1C[C@@H](NC(=O)C[C@H]2CCNC[C@H]2F)CN(c2ccc(C#N)c3ncncc23)C1]

The title compounds were prepared from 5-[(3R,5S)-3-amino-5-methylpiperidin-1-yl]quinazoline-8-carbonitrile and 2-{1-[(tert-butoxy)carbonyl]-3-fluoropiperidin-4-yl}acetic acid, followed by separation on chiral-HPLC under the following conditions: column, Repaired CHIRALPAK ID-3, 0.46×10 cm, 3 um; mobile phase, MtBE (with 0.1% DEA) in EtOH, 80% isocratic in 20 min; detector, UV 254 nm.

### Example 131

MS: 411 [M+H]⁺. ¹H NMR (400 MHz, DMSO-d₆, ppm) δ 9.61 (s, 1H), 9.36 (s, 1H), 8.41-8.33 (m, 1H), 8.06-7.95 (m, 1H), 7.30-7.21 (m, 1H), 4.52 (s, 0H), 4.40 (s, 0H), 4.10-3.91 (m, 0H), 3.85-3.76 (m, 1H), 3.67-3.57 (m, 1H), 3.10-2.99 (m, 1H), 2.90-2.79 (m, 1H), 2.76-2.59 (m, 3H), 2.50-2.37 (m, 2H), 2.28-1.91 (m, 5H), 1.37-1.04 (m, 3H), 0.94 (d, J=6.4H, 3H).

### Example 132

MS: 411 [M+H]⁺. ¹H NMR (400 MHz, DMSO-d₆, ppm) δ 9.61 (s, 1H), 9.36 (s, 1H), 8.37 (d, J=8.3 Hz, 1H), 8.0 (d, J=7.1 Hz, 1H), 7.25 (d, J=8.4 Hz, 1H), 4.46 (d, J=49.4 Hz, 1H), 4.04-4.0 (m, 1H), 3.85-3.78 (m, 1H), 3.67-3.59 (m, 1H), 3.09-2.98 (m, 1H), 2.92-2.84 (m, 1H), 2.76-2.55 (m, 5H), 2.25-2.15 (m, 1H), 2.13-1.89 (m, 4H), 1.37-1.32 (m, 2H), 1.22-1.09 (m, 1H), 0.94 (d, J=6.4 Hz, 3H).

### Example 133: 2-Amino-2-cyclopropyl-N-[(3R,5S)-5-methyl-1-(8-methyl-quinolin-5-yl)-piperidin-3-yl]-acetamide

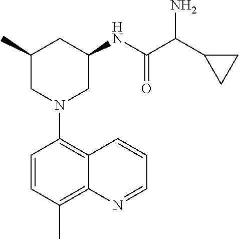 [SMILES: Cc1ccc(N2C[C@@H](C)C[C@@H](NC(=O)C(N)C3CC3)C2)c2cccnc12]

The title compound was prepared from (3R,5S)-5-Methyl-1-(8-methyl-quinolin-5-yl)-piperidin-3-ylamine hydrochloride and tert-Butoxycarbonylamino-cyclopropyl-acetic acid. MS: 353.5 [M+H]⁺. 1H NMR (400 MHz, DMSO-d₆) δ 8.91 (dd, J=4.1, 1.8 Hz, 1H), 8.48 (ddd, J=8.4, 1.8, 0.8 Hz, 1H), 7.67 (t, J=7.5 Hz, 1H), 7.55 (dd, J=8.5, 4.1 Hz, 1H), 7.52 (dt, J=7.6, 1.0 Hz, 1H), 7.08 (d, J=7.6 Hz, 1H), 4.05 (s, 1H), 3.17 (d, J=11.3 Hz, 1H), 2.69-2.64 (m, 3H), 2.36 (dtd, J=30.4, 10.9, 5.1 Hz, 3H), 1.98 (d, J=12.7 Hz, 1H), 1.67 (s, 1H), 1.09 (qd, J=12.0, 4.6 Hz, 1H), 0.95 (dd, J=6.5, 1.2 Hz, 3H), 0.88 (dddd, J=9.9, 7.8, 4.8, 2.4 Hz, 1H), 0.41-0.27 (m, 3H), 0.25-0.13 (m, 1H).

### Example 134: (2S,3R)-2-Amino-N-[(3R,5S)-1-(7-fluoro-8-methyl-quinolin-5-yl)-5-methyl-piperidin-3-yl]-3-hydroxy-butyramide

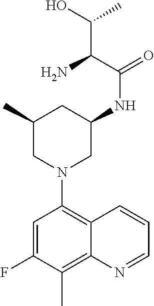 [SMILES: Cc1c(F)cc(N2C[C@@H](C)C[C@@H](NC(=O)[C@@H](N)[C@@H](C)O)C2)c2cccnc12]

The title compound was prepared from (3R,5S)-1-(7-Fluoro-8-methyl-quinolin-5-yl)-5-methyl-piperidin-3-ylamine hydrochloride and (2S,3R)-2-tert-Butoxycarbonylamino-3-hydroxy-butyric acid. MS: 375.5 [M+H]⁺. 1H NMR (400 MHz, DMSO-d₆) ? 8.97 (d, J=3.6 Hz, 1H), 8.61 (s, 1H), 8.50 (s, 1H), 8.10 (s, 3H), 7.56 (s, 1H), 7.07 (d, J=11.5 Hz, 1H), 4.10 (s, 1H), 3.88 (q, J=6.6 Hz, 1H), 3.45 (s, 3H), 3.39 (q, J=7.0 Hz, 1H), 3.24 (d, J=10.7 Hz, 1H), 2.55 (d, J=2.2 Hz, 3H), 2.48-2.37 (m, 1H), 2.03 (d, J=14.4 Hz, 2H), 1.20-1.03 (m, 5H), 0.96 (d, J=6.4 Hz, 3H).

### Example 135: (R)-2-Amino-N-[(3R,5S)-1-(7-fluoro-8-methyl-quinolin-5-yl)-5-methyl-piperidin-3-yl]-3-hydroxy-propionamide

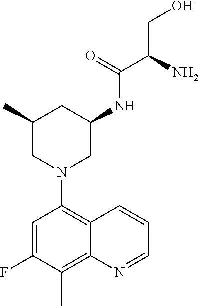 [SMILES: Cc1c(F)cc(N2C[C@@H](C)C[C@@H](NC(=O)[C@H](N)CO)C2)c2cccnc12]

The title compound was synthesized from (3R,5S)-1-(7-Fluoro-8-methyl-quinolin-5-yl)-5-methyl-piperidin-3-ylamine hydrochloride and boc-D-Ser-OH. MS: 361.4 [M+H]⁺. 1H NMR (400 MHz, Deuterium Oxide) δ 9.23 (dd, J=8.5, 1.6 Hz, 1H), 8.98 (dd, J=5.6, 1.5 Hz, 1H), 7.94 (dd, J=8.5, 5.6 Hz, 1H), 7.31 (d, J=11.5 Hz, 1H), 4.22 (s, 1H), 4.06 (dd, J=5.5, 4.1 Hz, 1H), 3.97-3.82 (m, 2H), 3.71 (s, 1H), 3.51 (d, J=8.7 Hz, 1H), 3.34 (d, J=11.9 Hz, 1H), 2.64 (t, J=11.0 Hz, 1H), 2.55 (d, J=1.7 Hz, 3H), 2.47 (t, J=11.5 Hz, 1H), 2.14 (d, J=11.5 Hz, 2H), 1.15 (q, J=12.6 Hz, 1H), 0.95 (d, J=6.3 Hz, 3H).

### Example 136: 2-(3-Fluoro-piperidin-4-yl)-N-[(3R,5S)-1-(8-methyl-[1,7]naphthyridin-5-yl)-5-trifluoromethyl-piperidin-3-yl]-acetamide

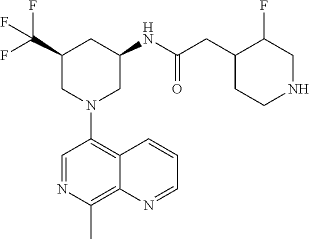 [SMILES: Cc1ncc(N2C[C@H](NC(=O)CC3CCNCC3F)C[C@H](C(F)(F)F)C2)c2cccnc12]

The title compound was synthesized from (3R,5S)-1-(8-Methyl-[1,7]naphthyridin-5-yl)-5-trifluoromethyl-piperidin-3-ylamine and 4-Carboxymethyl-3-fluoro-piperidine-1-carboxylic acid tert-butyl ester. MS: 454.3 [M+H]⁺.

### Example 137: 2-Fluoro-N-[(3R,5S)-1-(8-methyl-[1,7]naphthyridin-5-yl)-5-trifluoromethyl-piperidin-3-yl]-2-pyrrolidin-3-yl-acetamide

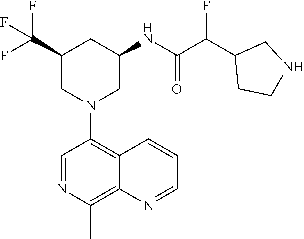 [SMILES: Cc1ncc(N2C[C@H](NC(=O)C(F)C3CCNC3)C[C@H](C(F)(F)F)C2)c2cccnc12]

The title compound was prepared from (3R,5S)-1-(8-Methyl-[1,7]naphthyridin-5-yl)-5-trifluoromethyl-piperidin-3-ylamine and 3-(Carboxy-fluoro-methyl)-pyrrolidine-1-carboxylic acid tert-butyl ester. MS: 439.2 [M+H]⁺. 1H NMR (400 MHz, DMSO-d₆) δ 8.98-8.94 (m, 1H), 8.49-8.44 (m, 1H), 8.32 (d, J=8.0 Hz, 1H), 7.88 (d, J=2.7 Hz, 1H), 7.83 (ddd, J=8.5, 4.2, 1.5 Hz, 1H), 7.41-7.36 (m, 1H), 4.20 (s, 2H), 4.04 (s, 3H), 2.90-2.64 (m, 6H), 2.11 (s, 2H), 1.65 (s, 3H), 1.15 (s, 3H), 0.84 (s, 3H).

### Example 138: N-[(3R,5S)-1-(8-Cyano-quinolin-5-yl)-5-trifluoromethyl-piperidin-3-yl]-2-(3-fluoro-piperidin-4-yl)-acetamide

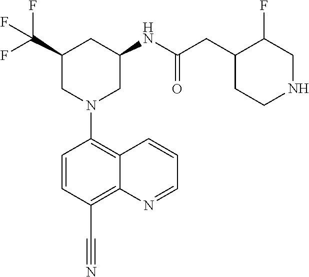 [SMILES: N#Cc1ccc(N2C[C@H](NC(=O)CC3CCNCC3F)C[C@H](C(F)(F)F)C2)c2cccnc12]

Prepared in an analogous manner from 5-((3R,5S)-3-Amino-5-trifluoromethyl-piperidin-1-yl)-quinoline-8-carbonitrile and 4-Carboxymethyl-3-fluoro-piperidine-1-carboxylic acid tert-butyl ester. MS: 464.3 [M+H]⁺. 1H NMR (400 MHz, DMSO-d₆) δ 9.06 (dd, J=4.2, 1.6 Hz, 1H), 8.58 (dd, J=8.6, 1.7 Hz, 1H), 8.25 (d, J=8.0 Hz, 1H), 8.09 (d, J=7.3 Hz, 1H), 7.72 (dd, J=8.5, 4.2 Hz, 1H), 7.32 (d, J=7.8 Hz, 1H), 4.15 (s, 2H), 3.57 (d, J=11.7 Hz, 2H), 3.20 (s, 2H), 2.94 (t, J=11.6 Hz, 2H), 2.13 (dt, J=49.4, 7.2 Hz, 5H), 1.50 (q, J=12.1 Hz, 2H), 1.30 (s, 3H).

### Example 139: 1-[(3R,5S)-1-(8-cyanoquinoxalin-5-yl)-5-(trifluoromethyl)piperidin-3-yl]-3-[2-(dimethylamino)ethyl]urea

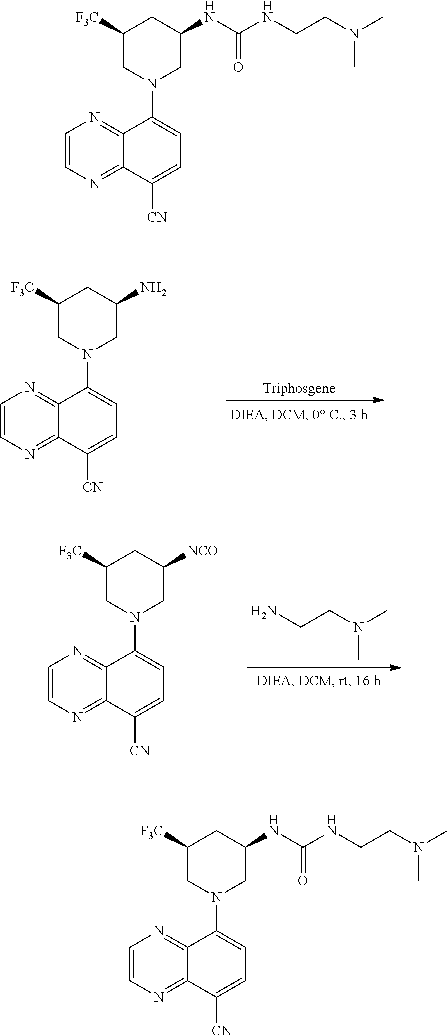

8-[(3R,5S)-3-isocyanato-5-(trifluoromethyl)piperidin-1-yl]quinoxaline-5-carbonitrile: At 0° C., to a solution of 8-[(3R,5S)-3-amino-5-(trifluoromethyl)piperidin-1-yl]quinoxaline-5-carbonitrile (94 mg, 0.29 mmol) and DIEA (115 mg, 0.89 mmol) in dichloromethane (8 mL) was added triphosgene (70 mg, 0.24 mmol) dropwise at 0° C. The resulting mixture was stirred for 3 h at 0° C., and then was concentrated under reduced pressure to yield 8-[(3R,5S)-3-isocyanato-5-(trifluoromethyl)piperidin-1-yl]quinoxaline-5-carbonitrile as a light yellow solid (54 mg, crude), which was used in next step without further purification. MS: 348.2 [M+H]⁺.

1-[(3R,5S)-1-(8-cyanoquinoxalin-5-yl)-5-(trifluoromethyl)piperidin-3-yl]-3-[2-(dimethylamino)ethyl]urea: To a solution of 8-[(3R,5S)-3-isocyanato-5-(trifluoromethyl)piperidin-1-yl]quinoxaline-5-carbonitrile (54 mg, crude) in dichloromethane (8 mL) was added DIEA (115 mg, 0.89 mmol) and (2-aminoethyl)dimethylamine (6 mg, 0.07 mmol) at room temperature. The resulting mixture was stirred for 16 h at room temperature. When the reaction was done, it was quenched by the addition of water (5 mL). The resulting mixture was extracted with ethyl acetate (30 mL×3). The organic phases were combined, washed with brine and dried over Na₂SO₄. The solvent was removed under reduced pressure and the residue was purified by prep-HPLC under the following conditions: column, XBridge Prep C18 OBD Column, 150×19 mm, 5 um; mobile phase, acetonitrile in water (with 10 mmol/L NH₄HCO₃ and 0.1% NH₃.H₂O), 15% to 40% gradient in 8 min; detector, UV 254 nm. 1-[(3R,5S)-1-(8-cyanoquinoxalin-5-yl)-5-(trifluoromethyl)piperidin-3-yl]-3-[2-(dimethylamino)ethyl]urea was obtained as a light yellow solid (23 mg, 18% for 2 steps). MS: 436 [M+H]⁺. H NMR (300 MHz, DMSO-d₆, ppm) δ 9.07 (d, J=1.8 Hz, 1H), 8.99 (d, J=1.8 Hz, 1H), 8.26 (d, J=8.4 Hz, 1H), 7.39 (d, J=8.5 Hz, 1H), 6.32 (d, J=7.3 Hz, 1H), 5.89-5.79 (m, 1H), 4.78-4.68 (m, 1H), 4.20-4.10 (m, 1H), 3.91-3.76 (m, 1H), 3.27-2.75 (m, 5H), 2.33-2.23 (m, 2H), 2.19-2.13 (m, 7H), 1.57-1.43 (m, 1H).

The following compound was synthesized in an analogous manner.

### Example 140: 1-[(3R,5S)-1-(8-cyanoquinoxalin-5-yl)-5-methylpiperidin-3-yl]-3-[2-(dimethylamino) ethyl]urea

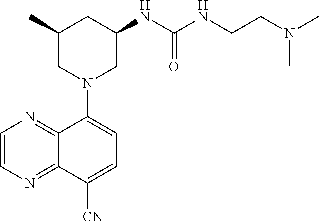 [SMILES: C[C@H]1C[C@@H](NC(=O)NCCN(C)C)CN(c2ccc(C#N)c3nccnc23)C1]

The title compound was prepared from 8-[(3R,5S)-3-amino-5-methylpiperidin-1-yl]quinoxaline-5-carbonitrile and (2-aminoethyl)dimethylamine. MS: 382 [M+H]⁺. H NMR (400 MHz, DMSO-d₆, ppm) δ 9.03 (d, J=1.8 Hz, 1H), 8.94 (d, J=1.8 Hz, 1H), 8.19 (d, J=8.4 Hz, 1H), 7.31 (d, J=8.6 Hz, 1H), 6.14 (d, J=7.3 Hz, 1H), 5.79-5.72 (m, 1H), 4.43-4.36 (m, 1 H), 4.30-4.22 (m, 1H), 3.72-3.68 (m, 1H), 3.16-3.0 (m, 2H), 2.75-2.63 (m, 2H), 2.29-2.22 (m, 2H), 2.14 (s, 6H), 2.0-1.76 (m, 2H), 1.12-0.98 (m, 1H), 0.90 (d, J=6.5 Hz, 3H).

### Example 141: 3-[2-(dimethylamino)ethyl]-1-[(3R,5S)-5-methyl-1-[8-(trifluoromethyl)quinoxalin-5-yl]piperidin-3-yl]urea

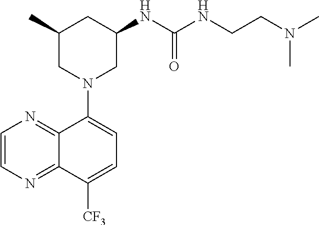 [SMILES: C[C@H]1C[C@@H](NC(=O)NCCN(C)C)CN(c2ccc(C(F)(F)F)c3nccnc23)C1]

The title compound was prepared from (3R,5S)-5-methyl-1-[8-(trifluoromethyl)quinoxalin-5-yl]piperidin-3-amine and (2-aminoethyl)dimethylamine. MS: 425 [M+H]⁺. ¹H NMR (300 MHz, DMSO-d₆, ppm) δ 9.01-8.95 (m, 2H), 8.06 (d, J=8.4 Hz, 1H), 7.29 (d, J=8.4 Hz, 1H), 6.12 (d, J=7.5 Hz, 1H), 5.81-5.71 (m, 1H), 4.26-4.10 (m, 2H), 3.77-3.71 (m, 1H), 3.19-3.01 (m, 2H), 2.66-2.52 (m, 2H), 2.32-2.22 (m, 2H), 2.15 (s, 6H), 1.98-1.92 (m, 2H), 1.10-0.96 (m, 1H), 0.92 (d, J=6.3 Hz, 3H).

### Example 142: 3-[2-(dimethylamino)ethyl]-1-[(3R,5S)-5-(trifluoromethyl)-1-[8-(trifluoromethyl)quinoxalin-5-yl]piperidin-3-yl]urea

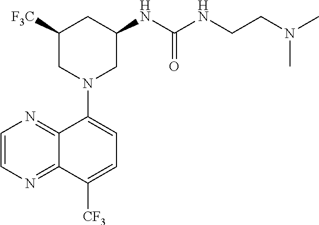 [SMILES: CN(C)CCNC(=O)N[C@@H]1C[C@H](C(F)(F)F)CN(c2ccc(C(F)(F)F)c3nccnc23)C1]

The title compound was prepared from (3R,5S)-5-(trifluoromethyl)-1-[8-(trifluoromethyl)quinoxalin-5-yl]piperidin-3-amine and (2-aminoethyl)dimethylamine. MS: 479 [M+H]⁺. ¹H NMR (300 MHz, DMSO-d₆, ppm) δ 9.04-8.98 (m, 2H), 8.10 (d, J=8.4 Hz, 1H), 7.37 (d, J=8.4 Hz, 1H), 6.30 (d, J=7.5 Hz, 1H), 5.83 (t, J=5.3 Hz, 1H), 4.59-4.50 (m, 1H), 4.12-4.01 (m, 1H), 3.90-3.84 (m, 1H), 3.22-2.85 (m, 4H), 2.75 (t, J=11.3 Hz, 1H), 2.32-2.22 (m, 2H), 2.22-2.19 (m, 1H), 2.15 (s, 6H), 1.53-1.36 (m, 1H).

### Example 143: 1-[(3R,5S)-1-(8-cyanoquinolin-5-yl)-5-(trifluoromethyl)piperidin-3-yl]-3-[2-(dimethylamino)ethyl]urea

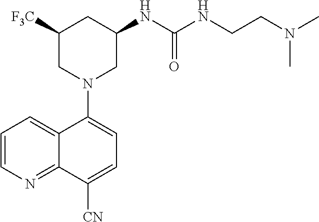 [SMILES: CN(C)CCNC(=O)N[C@@H]1C[C@H](C(F)(F)F)CN(c2ccc(C#N)c3ncccc23)C1]

The title compound was prepared from 5-[(3R,5S)-3-amino-5-(trifluoromethyl)piperidin-1-yl]quinoline-8-carbonitrile and (2-aminoethyl)dimethylamine. MS: 435 [M+H]⁺. ¹H NMR (400 MHz, DMSO-d₆, ppm) δ 9.09-9.03 (m, 1H), 8.58 (dd, J=8.6, 1.7 Hz, 1H), 8.25 (d, J=8.0 Hz, 1H), 7.71 (dd, J=8.6, 4.2 Hz, 1H), 7.31 (d, J=8.1 Hz, 1H), 6.28 (d, J=7.3 Hz, 1H), 5.79 (t, J=5.4 Hz, 1H), 3.99-3.94 (m, 1H), 3.59-3.56 (m, 2H), 3.25-3.01 (m, 3H), 2.93 (t, J=11.5 Hz, 1H), 2.57 (t, J=11.1 Hz, 1H), 2.31-2.17 (m, 3H), 2.12 (s, 6H), 1.46-1.33 (m, 1H).

### Example 144: 3-[2-(dimethylamino)ethyl]-1-[(3R,5S)-5-(trifluoromethyl)-1-[8-(trifluoromethyl)quinolin-5-yl]piperidin-3-yl]urea

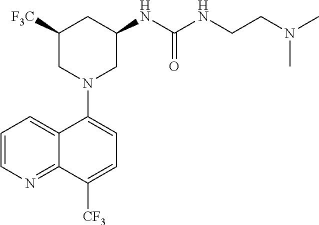 [SMILES: CN(C)CCNC(=O)N[C@@H]1C[C@H](C(F)(F)F)CN(c2ccc(C(F)(F)F)c3ncccc23)C1]

The title compound was prepared from (3R,5S)-5-(trifluoromethyl)-1-[8-(trifluoromethyl)quinolin-5-yl]piperidin-3-amine and (2-aminoethyl)dimethylamine. MS: 478 [M+H]⁺. ¹H NMR (300 MHz, DMSO-d₆, ppm) δ 9.08-9.0 (m, 1H), 8.60 (dd, J=8.6, 1.8 Hz, 1H), 8.08 (d, J=8.1 Hz, 1H), 7.71 (dd, J=8.6, 4.1 Hz, 1H), 7.32 (d, J=8.0 Hz, 1H), 6.31 (d, J=7.5 Hz, 1H), 5.83 (t, J=5.4 Hz, 1H), 4.02-3.96 (m, 1H), 3.55-3.46 (m, 2H), 3.27-3.03 (m, 3H), 2.87 (t, J=11.4 Hz, 1H), 2.61-2.51 (m, 1H), 2.36-2.25 (m, 2H), 2.25-2.20 (m, 1H), 2.17 (s, 6H), 1.49-1.30 (m, 1H).

### Example 145: 3-[2-(dimethylamino)ethyl]-1-[(3R,5S)-1-(7-fluoro-8-methylquinolin-5-yl)-5-methylpiperidin-3-yl]urea

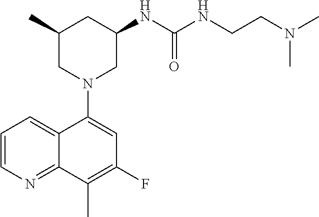 [SMILES: Cc1c(F)cc(N2C[C@@H](C)C[C@@H](NC(=O)NCCN(C)C)C2)c2cccnc12]

The title compound was prepared from (3R,5S)-1-(7-fluoro-8-methylquinolin-5-yl)-5-methylpiperidin-3-amine and (2-aminoethyl)dimethylamine. MS: 388 [M+H]⁺. ¹H NMR (300 MHz, DMSO-d₆, ppm) δ 8.98-8.90 (m, 1H), 8.44 (dd, J=8.5, 1.8 Hz, 1H), 7.53 (dd, J=8.5, 4.2 Hz, 1H), 7.04 (d, J=11.5 Hz, 1H), 6.09 (d, J=7.5 Hz, 1H), 5.73 (t, J=5.4 Hz, 1H), 3.88-3.79 (m, 1H), 3.48-3.39 (m, 1H), 3.25-3.16 (m, 1H), 3.16-2.98 (m, 2H), 2.57-2.51 (m, 3H), 2.41-2.21 (m, 4H), 2.13 (s, 6H), 2.03-1.93 (m, 2H), 1.02-0.84 (m, 4H).

### Example 146: 3-[(3R,5S)-5-methyl-1-[8-(trifluoromethyl)quinolin-5-yl]piperidin-3-yl]-1-[(3R)-piperidin-3-yl]urea

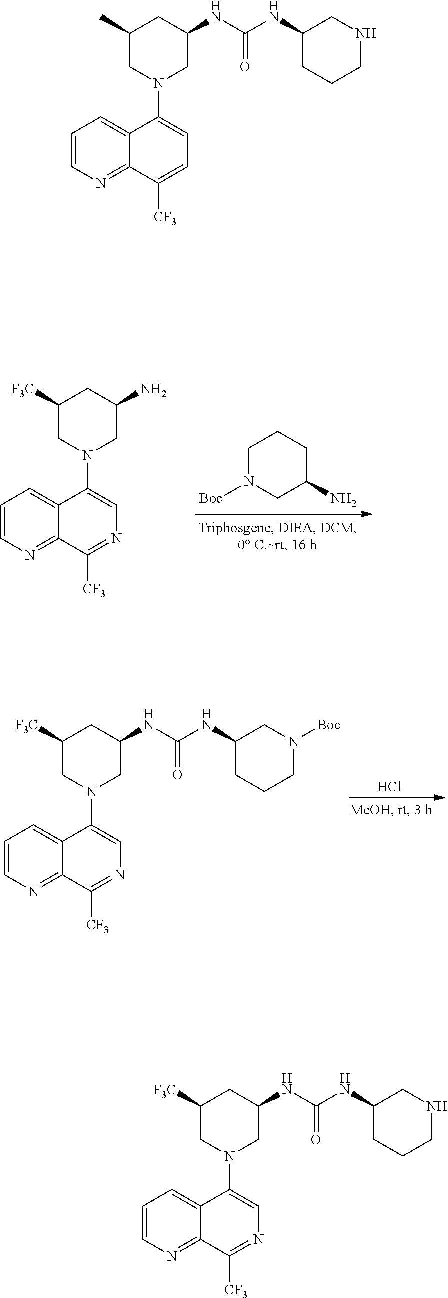 [SMILES: C[C@H]1C[C@@H](NC(=O)N[C@@H]2CCCNC2)CN(c2ccc(C(F)(F)F)c3ncccc23)C1 ; N[C@@H]1C[C@H](C(F)(F)F)CN(c2cnc(C(F)(F)F)c3ncccc23)C1 ; CC(C)(C)OC(=O)N1CCC[C@@H](N)C1 ; CC(C)(C)OC(=O)N1CCC[C@@H](NC(=O)N[C@@H]2C[C@H](C(F)(F)F)CN(c3cnc(C(F)(F)F)c4ncccc34)C2)C1 ; Cl ; O=C(N[C@@H]1CCCNC1)N[C@@H]1C[C@H](C(F)(F)F)CN(c2cnc(C(F)(F)F)c3ncccc23)C1]

tert-butyl (3R)-3-([[(3R,5S)-5-methyl-1-[8-(trifluoromethyl)quinolin-5-yl]piperidin-3-yl]carbamoyl]amino)piperidine-1-carboxylate: At 0° C., to a solution of (3R,5S)-5-methyl-1-[8-(trifluoromethyl)quinolin-5-yl]piperidin-3-amine (92 mg, 0.28 mmol) and DIEA (77 mg, 0.60 mmol) in dichloromethane (10 mL), was added a solution of triphosgene (29 mg, 0.10 mmol) in dichloromethane (5 mL) dropwise. The resulting mixture was stirred at 0° C. for 3 h, and then was added by tert-butyl (3R)-3-aminopiperidine-1-carboxylate (60 mg, 0.30 mmol). The resulting solution was stirred for additional 16 h at room temperature. The reaction mixture was concentrated under reduced pressure to yield tert-butyl (3R)-3-([[(3R,5S)-5-methyl-1-[8-(trifluoromethyl)quinolin-5-yl]piperidin-3-yl]carbamoyl]amino)piperidine-1-carboxylate as a light yellow solid (110 mg, crude), which was used in next step without further purification.

3-[(3R,5S)-5-methyl-1-[8-(trifluoromethyl)quinolin-5-yl]piperidin-3-yl]-1-[(3R)-piperidin-3-yl]urea: To a solution of tert-butyl (3R)-3-([[(3R,5S)-5-methyl-1-[8-(trifluoromethyl)quinolin-5-yl]piperidin-3-yl]carbamoyl]amino)piperidine-1-carboxylate (110 mg, crude) in methanol (10 mL) was added HCl aqueous solution (6 N, 3.3 mL, 19.99 mmol) at room temperature. The resulting mixture was stirred for 5 h at room temperature. After the reaction was done, the reaction mixture was concentrated under reduced pressure and the residue was purified by prep-HPLC under the following conditions: column, XBridge Prep C18 OBD Column, 150×19 mm, 5 um; mobile phase, acetonitrile in water (with 10 mmol/L NH₄HCO₃ and 0.1% NH₃.H₂O), 25% to 45% gradient in 8 min; detector, UV 254 nm. 3-[(3R,5S)-5-methyl-1-[8-(trifluoromethyl)quinolin-5-yl]piperidin-3-yl]-1-[(3R)-piperidin-3-yl]urea was obtained as a white solid (59 mg, 45% for 2 steps). MS: 436 [M+H]⁺. ¹H NMR (300 MHz, DMSO-d₆, ppm) δ 9.06-8.98 (m, 1H), 8.53 (dd, J=8.6, 1.8 Hz, 1H), 8.04 (d, J=8.1 Hz, 1H), 7.67 (dd, J=8.6, 4.2 Hz, 1H), 7.20 (d, J=8.1 Hz, 1H), 5.99-5.69 (m, 2H), 3.88-3.80 (m, 1H), 3.63-3.54 (m, 1H), 3.52-3.37 (m, 2H), 2.92-2.82 (m, 1H), 2.74-2.64 (m, 1H), 2.49-2.33 (m, 3H), 2.30-2.17 (m, 1H), 2.10-1.95 (m, 2H), 1.72-1.66 (m, 1H), 1.56-1.50 (m, 1H), 1.39-1.13 (m, 2H), 1.07-0.91 (m, 4H).

### Example 147: 1-(1-Methyl-piperidin-4-yl)-3-[(3R,5S)-5-methyl-1-(8-trifluoromethyl-quinolin-5-yl)-piperidin-3-yl]-urea

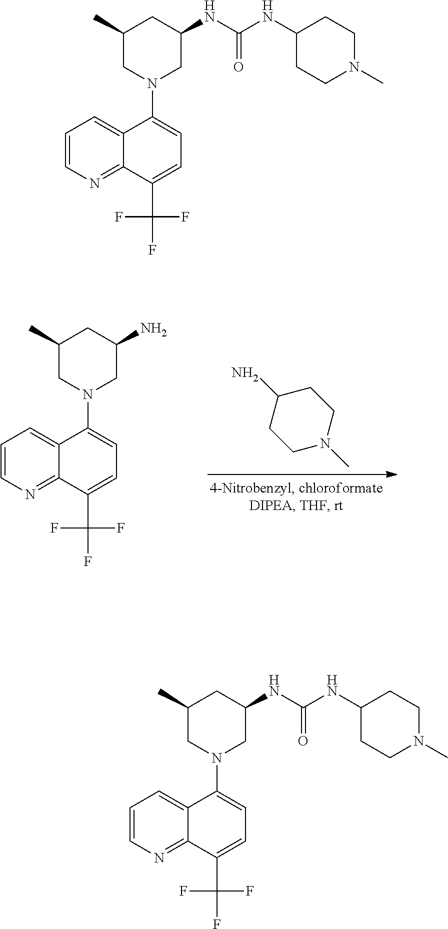

1-(1-Methyl-piperidin-4-yl)-3-[(3R,5S)-5-methyl-1-(8-trifluoromethyl-quinolin-5-yl)-piperidin-3-yl]-urea: In a scintillation vial, under nitrogen, (3R,5S)-5-Methyl-1-(8-trifluoromethyl-quinolin-5-yl)-piperidin-3-ylamine hydrochloride (2) (53.10 mg; 0.14 mmol; 1.0 eq.) was suspended in a solution of diisopropylethylamine (0.12 ml; 0.69 mmol; 5.0 eq.) in anhydrous THE (3.0 ml). The suspension was stirred at room temperature for 5 min then 4-nitrophenyl chloroformate (42.0 mg; 0.21 mmol; 1.50 eq.) was added. The reaction mixture was stirred for 2 hours, then 4-amino-1-methylpiperidine (0.03 mL; 0.28 mmol, 2.0 eq.) was added. The reaction was stirred overnight.

The reaction was concentrated to 1 mL and purified by prep-HPLC under the following conditions: Column, XBridge BEH130 Prep C18 OBD column, 19×150 mm 5 um 13 nm; Mobile phase, CAN/water with 0.10% NH4OH as modifier; Detector, UV 254 nm. The pure fraction was frozen and lyophilized to give 1-(1-Methyl-piperidin-4-yl)-3-[(3R,5S)-5-methyl-1-(8-trifluoromethyl-quinolin-5-yl)-piperidin-3-yl]-urea (31.40 mg; 0.07 mmol; 50.3%) as a white solid. MS: 450 [M+H]⁺. 1H NMR (400 MHz, DMSO-d₆) δ 9.0 (dd, J=4.2, 1.7 Hz, 1H), 8.52 (dd, J=8.6, 1.8 Hz, 1H), 8.03 (d, J=8.1 Hz, 1H), 7.66 (dd, J=8.6, 4.2 Hz, 1H), 7.19 (d, J=8.1 Hz, 1H), 5.76 (t, J=7.2 Hz, 2H), 3.85 (s, 1H), 3.34 (s, 5H), 2.60 (d, J=8.7 Hz, 2H), 2.40 (q, J=11.4 Hz, 2H), 2.12 (s, 3H), 1.95 (q, J=14.2, 11.2 Hz, 4H), 1.78-1.61 (m, 2H), 1.39-1.20 (m, 2H), 1.03-0.88 (m, 3H).

### Example 148: N-[(3R,5S)-1-(8-Cyano-[1,7]naphthyridin-5-yl)-5-methyl-piperidin-3-yl]-2-[3-fluoro-1-(2-hydroxy-ethyl)-piperidin-4-yl]-acetamide

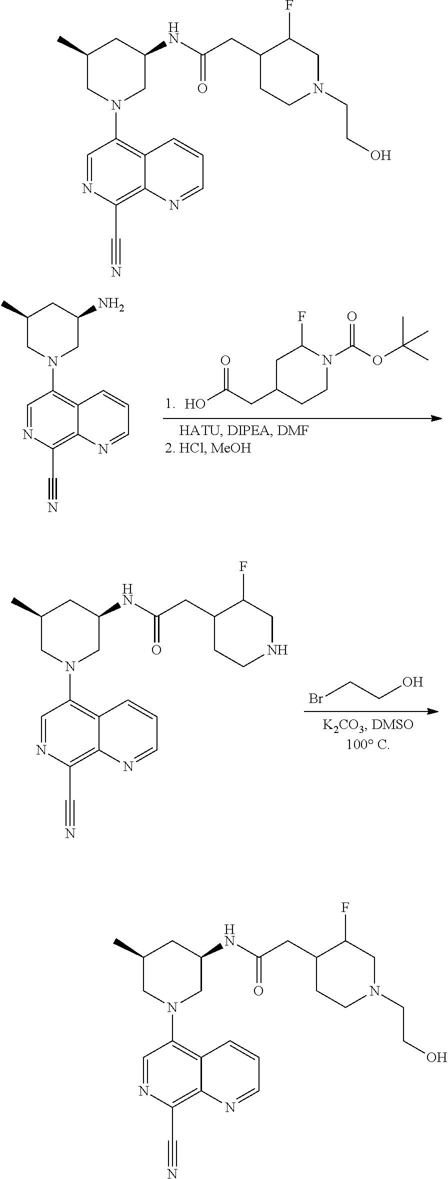

N-[(3R,5S)-1-(8-Cyano-[1,7]naphthyridin-5-yl)-5-methyl-piperidin-3-yl]-2-(3-fluoro-piperidin-4-yl)-acetamide: Title compound was prepared from (3R,5S)-1-(8-Cyano-naphthyridin-5-yl)-5-methyl-piperidin-3-ylamine and 4-Carboxymethyl-3-fluoro-piperidine-1-carboxylic acid tert-butyl ester in an analogous manner for Example 136.

N-[(3R,5S)-1-(8-Cyano-[1,7]naphthyridin-5-yl)-5-methyl-piperidin-3-yl]-2-[3-fluoro-1-(2-hydroxy-ethyl)-piperidin-4-yl]-acetamide: N-[(3R,5S)-1-(8-Cyano-[1,7]naphthyridin-5-yl)-5-methyl-piperidin-3-yl]-2-(3-fluoro-piperidin-4-yl)-acetamide (50 mg; 0.11 mmol; 1.0 eq.), 2-Bromo-ethanol (21 mg; 0.17 mmol; 1.50 eq.) and potassium carbonate (38 mg; 0.28 mmol; 2.50 eq.) were combined in a vial in DMSO (1 mL). The reaction was stirred at 100° C. overnight. The reaction was purified by prep HPLC with an acetonitrile/water (0.1% NH₄OH modified) gradient to obtain the title compound (28 mg; 0.06 mmol; 55.9%). MS: 455.4 [M+H]⁺. 1H NMR (400 MHz, DMSO-d₆) δ 9.39 (s, 1H), 9.17 (dd, J=4.1, 1.5 Hz, 1H), 8.55 (dd, J=8.7, 1.6 Hz, 1H), 8.39 (s, 1H), 8.10 (d, J=7.1 Hz, 1H), 7.87 (dd, J=8.7, 4.1 Hz, 1H), 5.26 (s, 1H), 4.04 (s, 1H), 3.75 (d, J=11.6 Hz, 4H), 3.62-3.53 (m, 1H), 3.15 (s, 3H), 2.65 (ddt, J=11.0, 7.2, 3.8 Hz, 2H), 2.31 (dt, J=12.7, 5.4 Hz, 2H), 2.16 (d, J=12.6 Hz, 2H), 2.07-1.95 (m, 2H), 1.77-1.64 (m, 2H), 1.15 (q, J=12.2 Hz, 1H), 0.96 (d, J=6.4 Hz, 3H).

### Example 149: 4-{[(3R,5S)-1-(8-Cyano-[1,7]naphthyridin-5-yl)-5-methyl-piperidin-3-ylcarbamoyl]-methyl}-3-fluoro-piperidine-1-carboxylic acid (2-hydroxy-1,1-dimethyl-ethyl)-amide

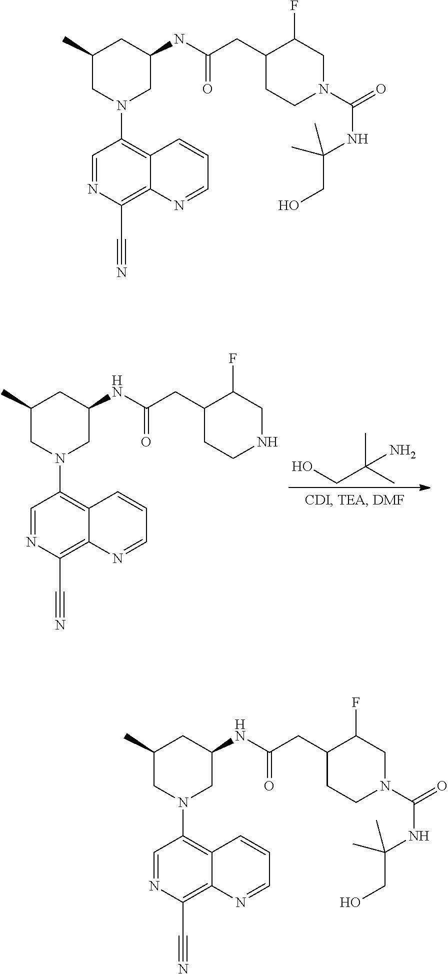

N-[(3R,5S)-1-(8-Cyano-[1,7]naphthyridin-5-yl)-5-methyl-piperidin-3-yl]-2-(3-fluoro-piperidin-4-yl)-acetamide (200 mg; 0.49 mmol; 1.0 eq.), 2-Amino-2-methyl-propan-1-ol (65 mg; 0.73 mmol; 1.50 eq.), and Di-imidazol-1-yl-methanone (158 mg; 0.97 mmol; 2.0 eq.) were added to a vial. Then, DMF (1 mL) and Triethyl-amine (147 mg; 1.46 mmol; 3.0 eq.) were added. The reaction was stirred for one hour. The crude was purified by prep HPLC with an acetonitrile/water gradient (0.1% NH₄OH modified) to obtain the title compound (18.5 mg; 0.04 mmol; 7.2%). MS: 526.4 [M+H]⁺. 1H NMR (400 MHz, DMSO-d₆) δ 9.17 (dd, J=4.1, 1.6 Hz, 1H), 8.55 (dd, J=8.7, 1.6 Hz, 1H), 8.38 (d, J=1.7 Hz, 1H), 8.04 (d, J=7.1 Hz, 1H), 7.93 (s, 2H), 7.87 (dd, J=8.7, 4.2 Hz, 1H), 4.21 (s, 2H), 4.01 (d, J=16.0 Hz, 3H), 3.75 (d, J=11.7 Hz, 1H), 3.06 (d, J=14.6 Hz, 2H), 2.83 (s, 1H), 2.70-2.60 (m, 2H), 2.32-2.21 (m, 1H), 1.99 (d, J=12.7 Hz, 2H), 1.49 (d, J=6.2 Hz, 1H), 1.38 (s, 1H), 1.25 (d, J=3.6 Hz, 6H), 1.15 (q, J=12.2 Hz, 2H), 0.96 (d, J=6.4 Hz, 3H).

### Example 150: 2-[(2-Amino-ethyl)-(2-hydroxy-ethyl)-amino]-N-[(3R,5S)-1-(8-cyano-quinoxalin-5-yl)-5-methyl-piperidin-3-yl]-acetamide

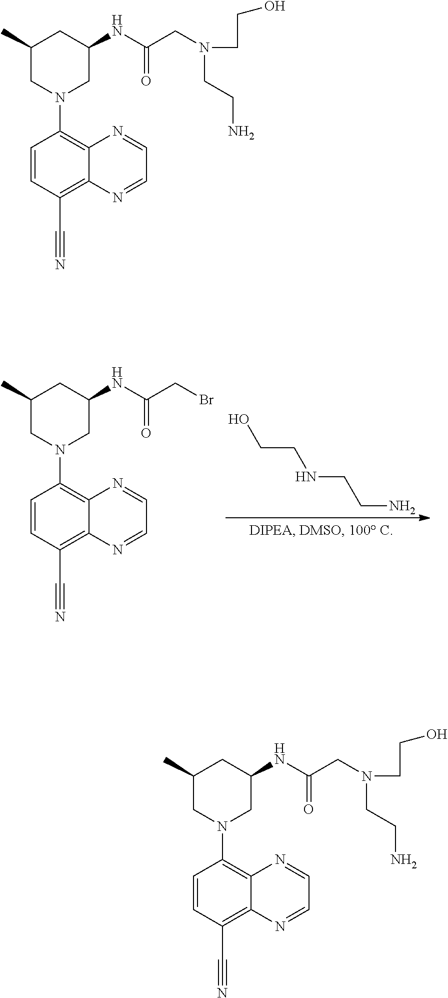

2-Bromo-N-[(3R,5S)-1-(8-cyano-quinoxalin-5-yl)-5-methyl-piperidin-3-yl]-acetamide: The title compound was prepared from 8-((3R,5S)-3-Amino-5-methyl-piperidin-1-yl)-quinoxaline-5-carbonitrile and Bromo-acetic acid in an analogous manner for Example 59.

2-[(2-Amino-ethyl)-(2-hydroxy-ethyl)-amino]-N-[(3R,5S)-1-(8-cyano-quinoxalin-5-yl)-5-methyl-piperidin-3-yl]-acetamide: 2-Bromo-N-[(3R,5S)-1-(8-cyano-quinoxalin-5-yl)-5-methyl-piperidin-3-yl]-acetamide (27 mg; 0.07 mmol; 1.0 eq.), 2-(2-Amino-ethylamino)-ethanol (9 mg; 0.08 mmol; 1.20 eq.) and Ethyl-diisopropyl-amine (19 mg; 0.21 mmol; 3.0 eq.) were combined in a vial in DMSO (1 mL). The reaction was heated to 100° C. overnight. Once the reaction was complete, it was purified by prep HPLC with an acetonitrile/water (0.1% NH₄OH modified) gradient to obtain the title compound (5.1 mg; 0.01 mmol; 17.8%). MS: 412.4 [M+H]⁺. 1H NMR (400 MHz, DMSO-d₆) δ 9.03 (s, 1H), 8.94 (s, 1H), 8.19 (d, J=8.2 Hz, 1H), 7.88 (d, J=7.9 Hz, 1H), 7.28 (d, J=8.9 Hz, 1H), 4.43 (s, 1H), 4.27 (dd, J=24.8, 13.0 Hz, 3H), 3.95 (s, 2H), 3.44 (s, 2H), 3.09 (s, 2H), 2.83 (t, J=11.5 Hz, 2H), 2.69 (t, J=11.4 Hz, 2H), 2.56 (d, J=5.3 Hz, 5H), 1.96 (d, J=13.9 Hz, 3H), 1.23 (d, J=12.0 Hz, 2H), 0.93 (d, J=6.2 Hz, 3H).

### Example 151: (3R,5S)-1-(8-Methoxy-[1,7]naphthyridin-5-yl)-5-methyl-piperidin-3-ylamine

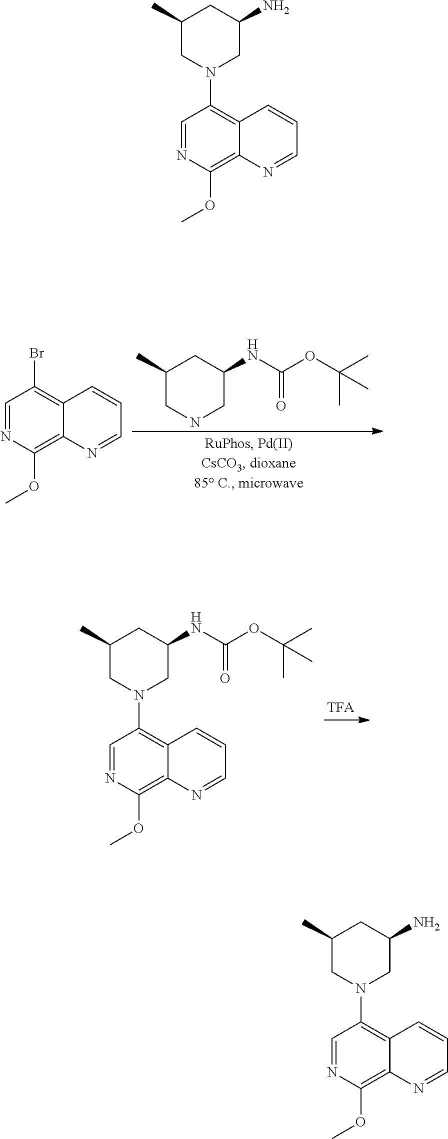

[(3R,5S)-1-(8-Methoxy-[1,7]naphthyridin-5-yl)-5-methyl-piperidin-3-yl]-carbamic acid tert-butyl ester: In a microwave vial, 5-Bromo-8-methoxy-[1,7]naphthyridine (0.58 g; 2.43 mmol; 1.0 eq.), ((3R,5S)-5-Methyl-piperidin-3-yl)-carbamic acid tert-butyl ester (0.62 g; 2.91 mmol; 1.20 eq.), chloro(2-dicyclohexylphosphino-2′,6′-di-i-propoxy-1,1′-biphenyl)[2-(2-aminoethylphenyl)]palladium(ii), methyl-t-butylether adduct (99 mg; 0.12 mmol; 0.05 eq.), 2-dicyclohexylphosphino-2′,6′-di-i-propoxy-1,1′-biphenyl (56 mg; 0.12 mmol; 0.05 eq.) and cesium carbonate (1.58 g; 4.85 mmol; 2.0 eq.) were dissolved in anhydrous Dioxane (11 ml). The reaction was placed under nitrogen and heated to 85° C. in the microwave for eight hours. The reaction was purified on silica with an ethyl acetate/hexanes gradient to afford the title compound (578 mg; 1.55 mmol; 64.0%). MS: 373.5 [M+H]⁺.

(3R,5S)-1-(8-Methoxy-[1,7]naphthyridin-5-yl)-5-methyl-piperidin-3-ylamine: [(3R,5S)-1-(8-Methoxy-[1,7]naphthyridin-5-yl)-5-methyl-piperidin-3-yl]-carbamic acid tert-butyl ester (185.0 mg; 0.50 mmol; 1.0 eq.) was dissolved in dioxane (2 mL) in a reaction vial. Trifluoroacetic acid (4 mL; 2.48 mmol; 5.0 eq.) was added, and the reaction stirred for four hours. The mixture was purified via prep HPLC with an acetonitrile/water (0.1% NH₄OH modified) gradient to afford the title compound (114.0 mg; 0.42 mmol; 84.3%). MS: 273.4 [M+H]⁺. 1HNMR (400 MHz, DMSO-d₆) δ 8.94 (dd, J=4.3, 2.1 Hz, 1H), 8.38-8.33 (m, 1H), 7.78 (dd, J=8.8, 4.0 Hz, 1H), 7.73 (s, 1H), 4.02 (d, J=1.8 Hz, 3H), 3.27-3.18 (m, 1H), 3.10 (d, J=11.4 Hz, 2H), 2.98 (s, 2H), 2.28 (t, J=10.8 Hz, 2H), 1.94 (s, 2H), 0.91 (d, J=6.3 Hz, 3H), 0.80 (q, J=12.1 Hz, 1H).

### Example 152: 5-{(3R,5S)-3-[(Piperidin-3-ylmethyl)-amino]-5-trifluoromethyl-piperidin-1-yl}-quinoline-8-carbonitrile

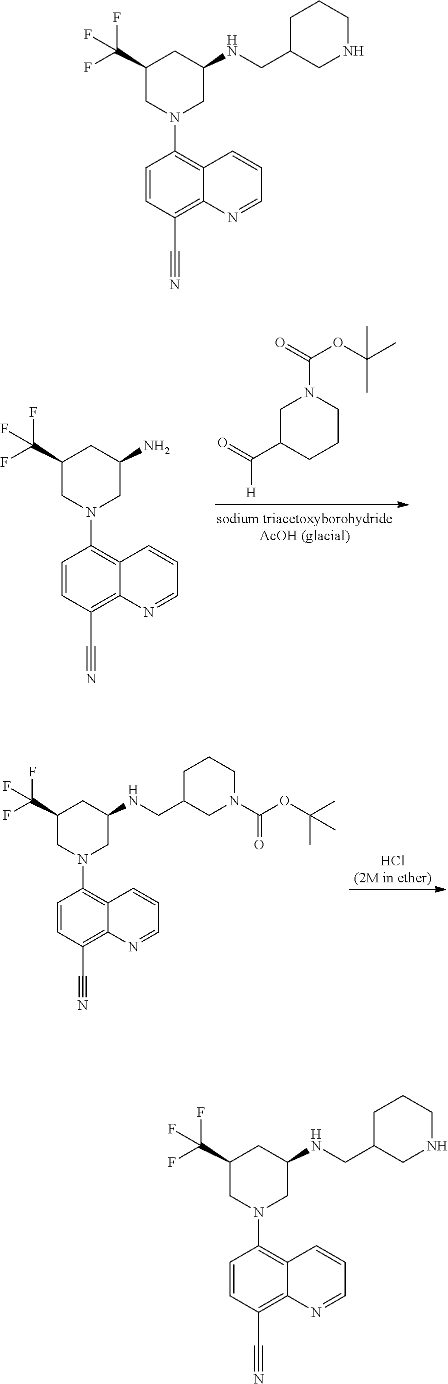 [SMILES: N#Cc1ccc(N2C[C@H](NCC3CCCNC3)C[C@H](C(F)(F)F)C2)c2cccnc12 ; N#Cc1ccc(N2C[C@H](N)C[C@H](C(F)(F)F)C2)c2cccnc12 ; CC(C)(C)OC(=O)N1CCCC(C=O)C1 ; CC(C)(C)OC(=O)N1CCCC(CN[C@@H]2C[C@H](C(F)(F)F)CN(c3ccc(C#N)c4ncccc34)C2)C1 ; N#Cc1ccc(N2C[C@H](NCC3CCCNC3)C[C@H](C(F)(F)F)C2)c2cccnc12]

3-{[(3R,5S)-1-(8-Cyano-quinolin-5-yl)-5-trifluoromethyl-piperidin-3-ylamino]-methyl}-piperidine-1-carboxylic acid tert-butyl ester: A solution of 5-((3R,5S)-3-Amino-5-trifluoromethyl-piperidin-1-yl)-quinoline-8-carbonitrile hydrochloride (3) (199.0 mg; 0.46 mmol; 1.0 eq.), tert-butyl 3-formylpiperidine-1-carboxylate (118.53 mg; 0.56 mmol; 1.20 eq.) and Acetic Acid (Glacial) (0.003 ml; 0.05 mmol; 0.10 eq.) in DCE (5 mL) was stirred for 1 hour, followed by the addition of sodium triacetoxyborohydride (147.23 mg; 0.69 mmol; 1.50 eq.). The resulting solution was stirred under argon at ambient temperature until complete. The crude product was purified on a flash system using a gradient of 20-100% EtOAc in hexanes to give 3-{[(3R,5S)-1-(8-Cyano-quinolin-5-yl)-5-trifluoromethyl-piperidin-3-ylamino]-methyl}-piperidine-1-carboxylic acid tert-butyl ester (72.30 mg; 0.14 mmol; 30.2%) as an oily residue after concentration. MS:518 [M+H]⁺.

5-{(3R,5S)-3-[(Piperidin-3-ylmethyl)-amino]-5-trifluoromethyl-piperidin-1-yl}-quinoline-8-carbonitrile: Into a round bottom flask with stir bar was dissolved 3-{[(3R,5S)-1-(8-Cyano-quinolin-5-yl)-5-trifluoromethyl-piperidin-3-ylamino]-methyl}-piperidine-1-carboxylic acid tert-butyl ester (72.30 mg; 0.14 mmol; 1.0 eq.) in minimal dichloromethane. The vial was sealed with a rubber septa, affixed with a Ar inlet, then hydrogen chloride (2M in Ether) (0.35 ml; 0.70 mmol; 5.0 eq.) was added. The reaction was allowed to stir until complete, as determined by LCMS analysis. The crude was purified on prep HPLC under basic conditions to give the title compound (25 mg, 0.06 mmol, 42.8%) as a white fluffy solid after lyophilization. MS:418 [M+H]⁺. ¹H NMR (400 MHz, DMSO-d₆) δ 9.05 (dd, J=4.2, 1.6 Hz, 1H), 8.50 (dd, J=8.6, 1.7 Hz, 1H), 8.24 (d, J=8.0 Hz, 1H), 7.71 (dd, J=8.6, 4.2 Hz, 1H), 7.30 (d, J=8.1 Hz, 1H), 3.55 (t, J=12.2 Hz, 2H), 3.05 (s, 1H), 2.99-2.76 (m, 3H), 2.38 (dd, J=19.1, 8.4 Hz, 2H), 2.27 (d, J=12.4 Hz, 1H), 2.11 (s, 1H), 1.74 (d, J=13.0 Hz, 1H), 1.56-1.36 (m, 2H), 1.36-1.18 (m, 2H), 1.04-0.90 (m, 1H).

### Example 153: 8-[cis-3-methyl-5-(1H-1,2,3-triazol-1-yl)piperidin-1-yl]quinoxaline-5-carbonitrile

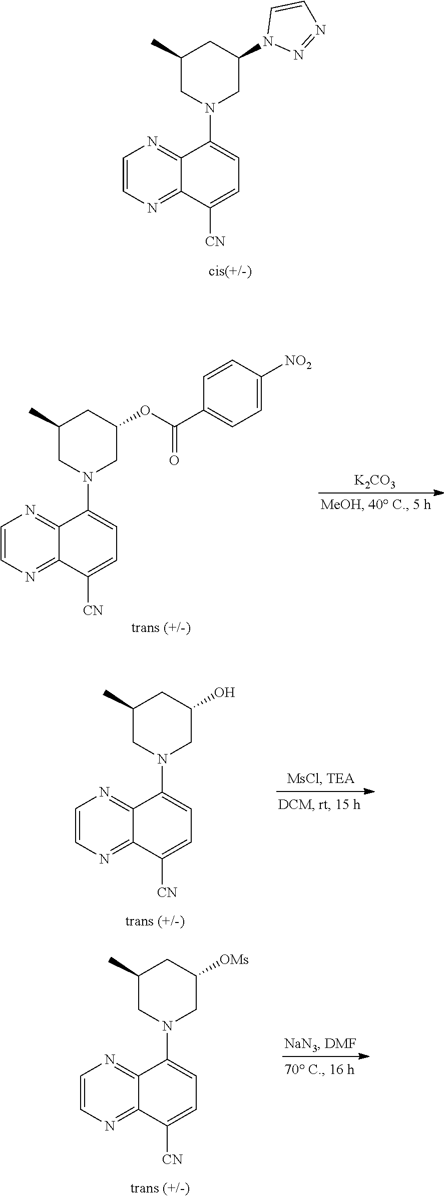 [SMILES: C[C@H]1C[C@@H](n2ccnn2)CN(c2ccc(C#N)c3nccnc23)C1 ; C[C@H]1C[C@H](OC(=O)c2ccc([N+](=O)[O-])cc2)CN(c2ccc(C#N)c3nccnc23)C1 ; "K2CO3" ; C[C@H]1C[C@H](O)CN(c2ccc(C#N)c3nccnc23)C1 ; C[C@H]1C[C@H](OS(C)(=O)=O)CN(c2ccc(C#N)c3nccnc23)C1]

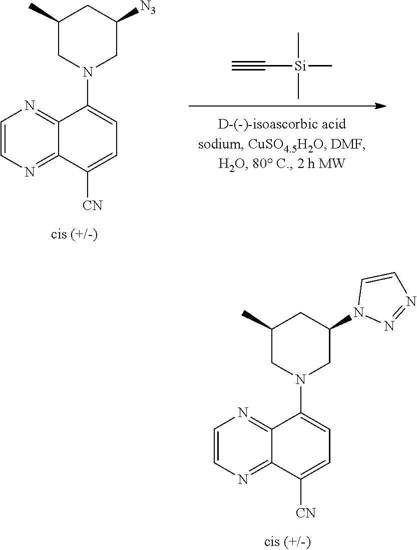

8-[trans-3-hydroxy-5-methylpiperidin-1-yl]quinoxaline-5-carbonitrile: To a solution of trans-1-(8-cyanoquinoxalin-5-yl)-5-methylpiperidin-3-yl 4-nitrobenzoate (324 mg, 0.78 mmol) in methanol (20 mL) was added potassium carbonate (324 mg, 2.36 mmol) at room temperature. The resulting mixture was stirred for 5 h at 40° C. When the reaction was done, the solids were filtered out and the filtrate was concentrated under reduced pressure to yield 8-[trans-3-hydroxy-5-methylpiperidin-1-yl]quinoxaline-5-carbonitrile as a yellow solid (200 mg, crude). MS: 269.0 [M+H]⁺.

trans-1-(8-cyanoquinoxalin-5-yl)-5-methylpiperidin-3-yl methanesulfonate: At 0° C., to a solution of 8-[trans-3-hydroxy-5-methylpiperidin-1-yl]quinoxaline-5-carbonitrile (200 mg, crude) in dichloromethane (15 mL) was added TEA (215 mg, 2.12 mmol), MsCl (98 mg, 0.85 mmol) in sequence. The resulting mixture was stirred for 15 h at room temperature. When the reaction was done, the reaction was then quenched by the addition of water (10 mL). The resulting mixture was extracted with ethyl acetate (30 mL×3). The organic phases were combined, washed with brine and dried over Na₂SO₄. The solvent was removed under reduced pressure and the residue was purified by flash chromatography eluting with EtOAc in hexane (0% to 66% gradient) to yield trans-1-(8-cyanoquinoxalin-5-yl)-5-methylpiperidin-3-yl methanesulfonate as a yellow solid (170 mg, 63% for 2 steps). MS: 347.0 [M+H]⁺.

8-[cis-3-azido-5-methylpiperidin-1-yl]quinoxaline-5-carbonitrile: To a solution of trans-1-(8-cyanoquinoxalin-5-yl)-5-methylpiperidin-3-yl methanesulfonate (156 mg, 0.45 mmol) in N,N-dimethylformamide (10 mL) was added NaN₃ (61 mg, 0.94 mmol) at room temperature. The resulting mixture was stirred for 16 h at 70° C. When the reaction was done, it was quenched by the addition of sat. sodium bicarbonate solution (30 mL). The resulting mixture was extracted with ethyl acetate (50 mL×3). The organic phases were combined, washed with brine and dried over Na₂SO₄. The solvent was removed under reduced pressure and the residue was purified by flash chromatography eluting with EtOAc in hexane (0% to 50% gradient) to yield 8-[cis-3-azido-5-methylpiperidin-1-yl]quinoxaline-5-carbonitrile as a yellow solid (68 mg, 51%). MS: 294.3 [M+H]⁺.

8-[cis-3-methyl-5-(1H-1,2,3-triazol-1-yl)piperidin-1-yl]quinoxaline-5-carbonitrile: To a solution of 8-[cis-3-azido-5-methylpiperidin-1-yl]quinoxaline-5-carbonitrile (68 mg, 0.23 mmol) in N,N-dimethylformamide (2 mL) was added ethynyltrimethylsilane (48 mg, 0.48 mmol), sodium (2R)-2-[(1R)-1,2-dihydroxyethyl]-4-hydroxy-5-oxo-2,5-dihydrofuran-3-olate (19 mg, 0.10 mmol) and a solution of CuSO₄.5H₂O (6 mg, 0.02 mmol) in water (0.6 mL) at room temperature under nitrogen atmosphere. The resulting mixture was irradiated with microwave for 2 h at 80° C. under nitrogen atmosphere. When the reaction was done, the reaction mixture was diluted with water (20 mL). The resulting mixture was extracted with ethyl acetate (30 mL×3). The organic phases were combined, washed with brine and dried over Na₂SO₄. The solvent was removed under reduced pressure and the residue was purified by prep-HPLC under the following conditions: column, XBridge Prep C18 OBD Column, 150×19 mm, 5 um; mobile phase, acetonitrile in water (with 10 mmol/L NH₄HCO₃ and 0.1% NH₃.H₂O), 25% to 49% gradient in 7 min; detector, UV 254 nm. 8-[cis-3-methyl-5-(1H-1,2,3-triazol-1-yl)piperidin-1-yl]quinoxaline-5-carbonitrile was obtained as a orange solid (35 mg, 47%). MS: 320.0 [M+H]⁺.

¹H NMR (400 MHz, DMSO-d₆) δ 9.05 (d, J=1.7 Hz, 1H), 8.98 (d, J=1.8 Hz, 1H), 8.32-8.17 (m, 2H), 7.77 (d, J=1.0 Hz, 1H), 7.33 (d, J=8.4 Hz, 1H), 5.01-4.89 (m, 1H), 4.73-4.63 (m, 1H), 4.21-4.12 (m, 1H), 3.37 (t, J=11.6 Hz, 1H), 2.83 (t, J=11.9 Hz, 1H), 2.38-2.29 (m, 1H), 2.21-2.07 (m, 1H), 1.93-1.78 (m, 1H), 1.03 (d, J=6.5 Hz, 3H).

The following compound was synthesized in an analogous manner.

### Example 154: 5-[cis-3-methyl-5-(1H-1,2,3-triazol-1-yl)piperidin-1-yl]-8-(trifluoromethyl)quinoxaline

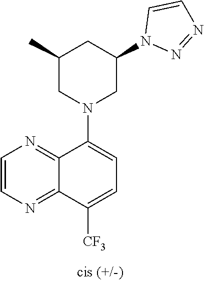 [SMILES: C[C@H]1C[C@@H](n2ccnn2)CN(c2ccc(C(F)(F)F)c3nccnc23)C1]

The title compound was prepared from trans-5-methyl-1-[8-(trifluoromethyl)quinoxalin-5-yl]piperidin-3-yl 4-nitrobenzoate. MS: 363.0 [M+H]⁺. ¹H NMR (300 MHz, DMSO-d₆) δ 9.03 (s, 1H), 8.99 (s, 1H), 8.27 (s, 1H), 8.08 (d, J=8.4 Hz, 1H), 7.77 (s, 1H), 7.33 (d, J=8.4 Hz, 1H), 5.01-4.95 (m, 1H), 4.58-4.47 (m, 1H), 4.07-4.03 (m, 1H), 3.28-3.21 (m, 1H), 2.78-2.71 (m, 1H), 2.39-2.29 (m, 1H), 2.20-2.14 (m, 1H), 1.92-1.73 (m, 1H), 1.04 (d, J=6.5 Hz, 3H).

### Example 155: 5-[cis-3-methyl-5-(1H-pyrazol-1-yl)piperidin-1-yl]-8-(trifluoromethyl)quinoxaline

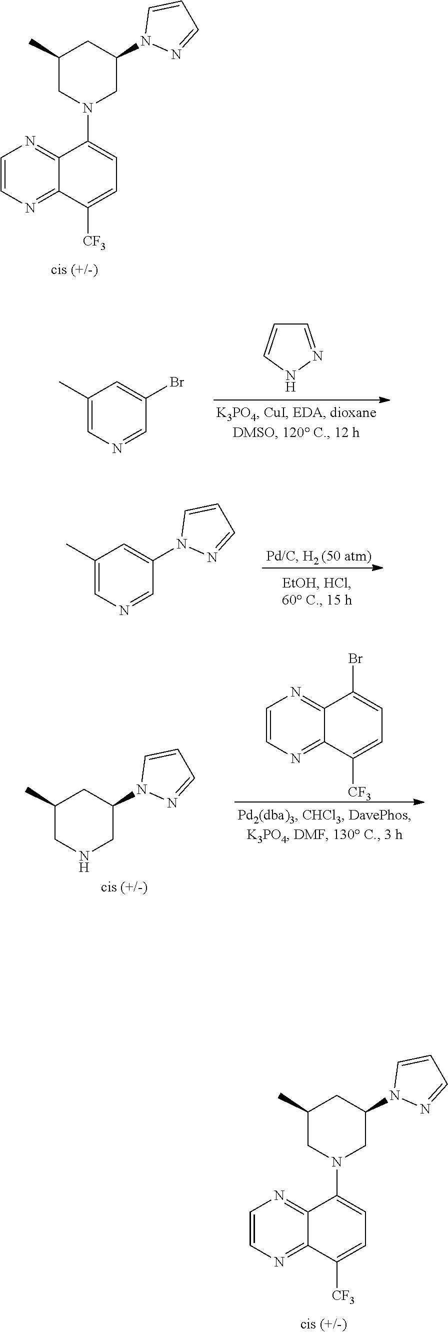

3-methyl-5-(1H-pyrazol-1-yl)pyridine: In a 150 mL sealed tube, to a solution of 3-bromo-5-methylpyridine (4.75 g, 27.61 mmol) in dioxane (60 mL) and DMSO (15 mL) was added 1H-pyrazole (5.65 g, 83.03 mmol), K₃PO₄ (11.73 g, 55.27 mmol), CuI (523 mg, 2.74 mmol), ethane-1,2-diamine (166 mg, 2.77 mmol) at room temperature under nitrogen atmosphere. The reaction mixture was stirred for 12 h at 120° C. under nitrogen atmosphere. After the reaction was done, the reaction mixture was concentrated under reduced pressure and the residue was purified by flash chromatography eluting with EtOAc in hexane (0% to 10% gradient) to yield 3-methyl-5-(1H-pyrazol-1-yl)pyridine as a light yellow solid (4.0 g, 90%). MS: 159.9 [M+H]⁺.

cis-3-methyl-5-(1H-pyrazol-1-yl)piperidine: At room temperature, to a solution of 3-methyl-5-(1H-pyrazol-1-yl)pyridine (3.0 g, 18.73 mmol) in EtOH (300 mL) was added palladium carbon (950 mg, 8.93 mmol) and hydrogen chloride solution (12 N, 20 mL, 240 mmol) under nitrogen atmosphere. The reaction tank was vacuumed and flushed with hydrogen. The reaction mixture was hydrogenated for 12 h at 60° C. under hydrogen atmosphere (50 atm). After the reaction was done, the reaction mixture was filtered through a Celite pad and the pH value of the filtrate was adjusted to 9 with NH₃ in MeOH solution (7 M). The resulting mixture was concentrated under reduced pressure and the residue was purified by flash chromatography eluting with MeOH in DCM (0% to 20% gradient) to yield cis-3-methyl-5-(1H-pyrazol-1-yl)piperidine as brown oil (1.52 g, 49%). MS: 166.2 [M+H]⁺.

8-[cis-3-methyl-5-(1H-1,2,3-triazol-1-yl)piperidin-1-yl]quinoxaline-5-carbonitrile: To a solution of 5-bromo-8-(trifluoromethyl)quinoxaline (143 mg, 0.51 mmol) in DMF (5 mL) was added cis-3-methyl-5-(1H-pyrazol-1-yl)piperidine (170 mg, 1.03 mmol), Pd₂(dba)₃.CHCl₃ (53 mg, 0.05 mmol), K₃PO₄ (327 mg, 1.54 mmol), DavePhos (40 mg, 0.10 mmol) at room temperature under nitrogen atmosphere. The reaction mixture was irradiated with microwave radiation for 3 h at 130° C. under nitrogen atmosphere. When the reaction was done, the reaction mixture was then diluted with water (5 mL). The resulting mixture was extracted with ethyl acetate (30 mL×3). The organic phases were combined, washed with brine and dried over Na₂SO₄. The solvent was removed under reduced pressure and the residue was purified by prep-HPLC under the following conditions: column, XBridge Prep C18 OBD Column, 150×19 mm, 5 um; mobile phase, acetonitrile in water (with 10 mmol/L NH₄HCO₃ and 0.10% NH₃.H₂O), 30% to 55% gradient in 7 min; detector, UV 254 nm. 5-[cis-3-methyl-5-(1H-pyrazol-1-yl)piperidin-1-yl]-8-(trifluoromethyl)quinoxaline was obtained as a yellow solid (27 mg, 14%). MS: 362.0 [M+H]⁺. ¹H NMR (400 MHz, DMSO-d₆) δ 9.02 (d, J=1.8 Hz, 1H), 8.98 (d, J=1.8 Hz, 1H), 8.06 (d, J=8.5 Hz, 1H), 7.85 (d, J=2.3 Hz, 1H), 7.47 (d, J=1.8 Hz, 1H), 7.29 (d, J=8.4 Hz, 1H), 6.29-6.23 (m, 1H), 4.68-4.58 (m, 1H), 4.47-4.40 (m, 1H), 4.10-4.02 (m, 1H), 3.18 (t, J=11.4 Hz, 1H), 2.71 (t, J=11.6 Hz, 1H), 2.28-2.20 (m, 1H), 2.14-2.09 (m, 1H), 1.84-1.70 (m, 1H), 1.02 (d, J=6.6 Hz, 3H).

### Example 156: 8-[cis-3-(1H-pyrazol-1-yl)-5-(trifluoromethyl)piperidin-1-yl]quinoxaline-5-carbonitrile

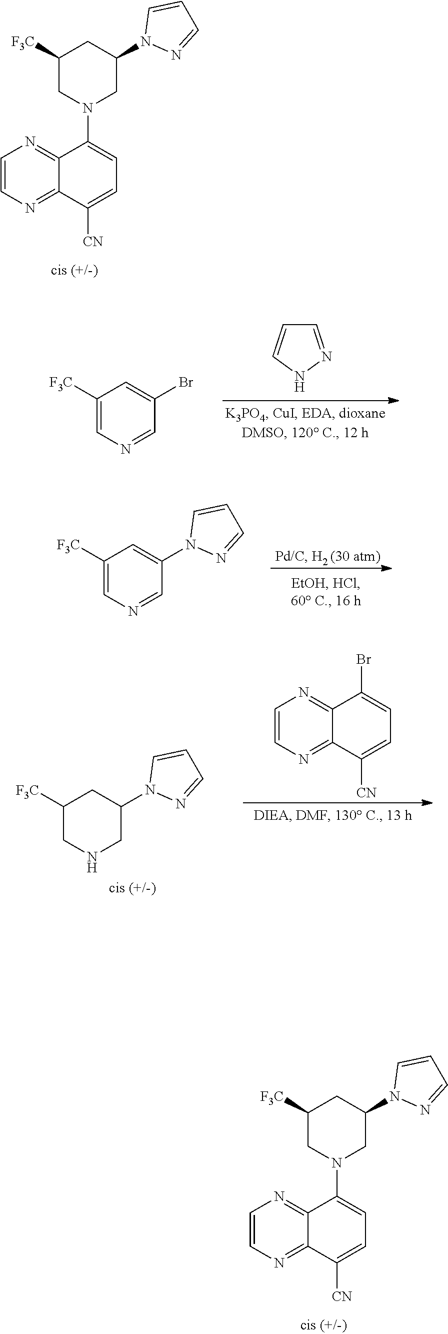

3-(1H-pyrazol-1-yl)-5-(trifluoromethyl)pyridine: In a 50 mL sealed tube, to a solution of 3-bromo-5-(trifluoromethyl)pyridine (2.85 g, 12.61 mmol) in dioxane (16 mL) and DMSO (4 mL) was added 1H-pyrazole (2.47 g, 36.28 mmol), ethane-1,2-diamine (73 mg, 1.22 mmol), K₃PO₄ (5.21 g, 24.57 mmol), CuI (234 mg, 1.23 mmol) at room temperature under nitrogen atmosphere. The reaction mixture was stirred for 12 h at 120° C. under nitrogen atmosphere. After the reaction was done, the reaction mixture was concentrated under reduced pressure and the residue was purified by flash chromatography eluting with EtOAc in hexane (0% to 20% gradient) to yield 3-methyl-5-(1H-pyrazol-1-yl)pyridine as a white solid (1.17 g, 44%). MS: 213.9 [M+H]⁺.

3-(1H-pyrazol-1-yl)-5-(trifluoromethyl)piperidine: At room temperature, to a solution of 3-(1H-pyrazol-1-yl)-5-(trifluoromethyl)pyridine (900 mg, 4.22 mmol) in ethanol (mL) was added hydrogen chloride solution (6 N, 2 mL, 12.0 mmol) and palladium carbon (30 mg, 0.27 mmol) under nitrogen atmosphere. The reaction tank was vacuumed and flushed with hydrogen. The reaction mixture was hydrogenated for 16 h at 60° C. under hydrogen atmosphere (30 atm). After the reaction was done, the reaction mixture was filtered through a Celite pad and the pH value of the filtrate was adjusted to 9 with NH₃ in MeOH solution (7 M). The resulting mixture was concentrated under reduced pressure and the residue was purified by flash chromatography eluting with MeOH in DCM (0% to 15% gradient) to yield 3-(1H-pyrazol-1-yl)-5-(trifluoromethyl)piperidine as light yellow oil (480 mg, cis/trans=4:1, 52%). MS: 220.2 [M+H]⁺.

8-[cis-3-(1H-pyrazol-1-yl)-5-(trifluoromethyl)piperidin-1-yl]quinoxaline-5-carbonitrile: To a solution of 58-bromoquinoxaline-5-carbonitrile (95 mg, 0.41 mmol) in N,N-dimethylformamide (5 mL) was added 3-(1H-pyrazol-1-yl)-5-(trifluoromethyl)piperidine (75 mg, 0.34 mmol), DIEA (253 mg, 1.96 mmol) at room temperature under nitrogen atmosphere. The reaction mixture was irradiated with microwave for 13 h at 130° C. under nitrogen atmosphere. When the reaction was done, the reaction mixture was then diluted with water (5 mL). The resulting mixture was extracted with ethyl acetate (30 mL×3). The organic phases were combined, washed with brine and dried over Na₂SO₄. The solvent was removed under reduced pressure and the residue was purified by prep-HPLC under the following conditions: column, XBridge Prep C18 OBD Column, 150×19 mm, 5 um; mobile phase, acetonitrile in water (with 10 mmol/L NH₄HCO₃ and 0.1% NH₃.H₂O), 35% to 59% gradient in 7 min; detector, UV 254 nm. 8-[cis-3-(1H-pyrazol-1-yl)-5-(trifluoromethyl)piperidin-1-yl]quinoxaline-5-carbonitrile was obtained as a yellow solid (24 mg, 16%). MS: 373.0 [M+H]⁺. H NMR (400 MHz, DMSO-d₆) δ 9.08 (d, J=1.8 Hz, 1H), 9.02 (d, J=1.8 Hz, 1H), 8.26 (d, J=8.3 Hz, 1H), 7.92 (d, J=2.3 Hz, 1H), 7.53 (d, J=1.8 Hz, 1H), 7.39 (d, J=8.4 Hz, 1H), 6.34-6.28 (m, 1H), 4.83-4.71 (m, 1H), 4.56-4.49 (m, 1H), 4.47-4.39 (m, 1H), 3.41 (t, J=11.6 Hz, 1H), 3.27-3.07 (m, 2H), 2.50-2.42 (m, 1H), 2.30-2.08 (m, 1H).

### Example 157: 5-[(3R,5S)-3-(1H-imidazol-1-yl)-5-methylpiperidin-1-yl]-8-(trifluoromethyl)quinoxaline

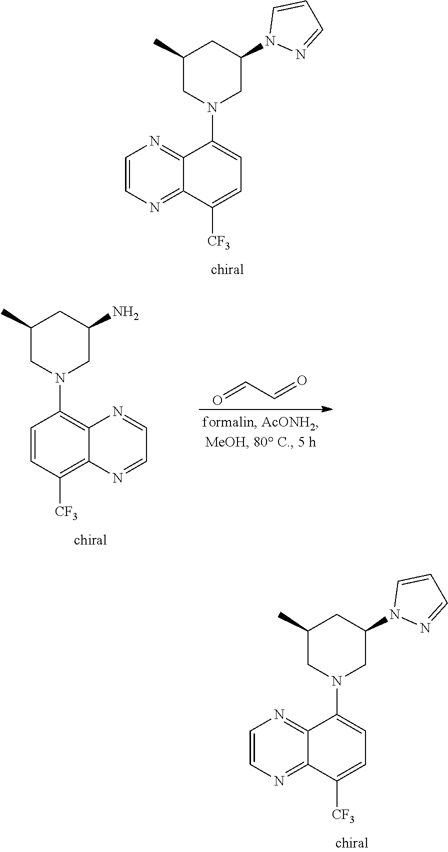

5-[(3R,5S)-3-(1H-imidazol-1-yl)-5-methylpiperidin-1-yl]-8-(trifluoromethyl)quinoxaline: To a solution of (3R,5S)-5-methyl-1-[8-(trifluoromethyl)quinoxalin-5-yl]piperidin-3-amine (94 mg, 0.30 mmol) in MeOH (4 mL) was added oxaldehyde (74 mg, 1.28 mmol), formalin (37%, 97 mg, 1.20 mmol), CH₃COONH₄ (95 mg, 1.22 mmol) at room temperature. The reaction mixture was stirred for 5 h at 80° C. When the reaction was done, the reaction mixture was then quenched by the addition of KOH solution (1 N, 3 mL). The resulting mixture was extracted with ethyl acetate (20 mL×3). The organic phases were combined, washed with brine and dried over Na₂SO₄. The solvent was removed under reduced pressure and the residue was purified by prep-HPLC under the following conditions: column, XBridge Prep C18 OBD Column, 150×19 mm, 5 um; mobile phase, acetonitrile in water (with 10 mmol/L NH₄HCO₃ and 0.10% NH₃.H₂O), 39% to 45% gradient in 7 min; detector, UV 254 nm. The title compound was obtained as a yellow solid (35 mg, 32%). MS: 362.0 [M+H]⁺. ¹H NMR (300 MHz, DMSO-d₆) δ 9.03 (d, J=1.8 Hz, 1H), 8.99 (d, J=1.5 Hz, 1H), 8.05 (d, J=8.4 Hz, 1H), 7.78 (s, 1H), 7.37-7.25 (m, 2H), 6.91 (s, 1H), 4.57-4.51 (m, 1H), 4.41-4.31 (m, 1H), 4.10-4.0 (m, 1H), 3.17 (t, J=11.3 Hz, 1H), 2.68 (t, J=11.6 Hz, 1H), 2.30-1.94 (m, 2H), 1.79-1.61 (m, 1H), 1.01 (d, J=6.5 Hz, 3H).

The following compounds were synthesized in an analogous manner.

### Example 158: 5-[(3R,5S)-3-(1H-imidazol-1-yl)-5-methylpiperidin-1-yl]quinoline-8-carbonitrile

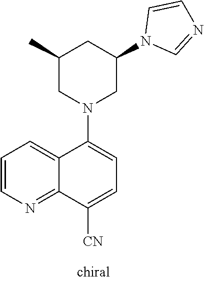 [SMILES: C[C@H]1C[C@@H](n2ccnc2)CN(c2ccc(C#N)c3ncccc23)C1]

The title compound was prepared from 5-[(3R,5S)-3-amino-5-methylpiperidin-1-yl]quinoline-8-carbonitrile, oxaldehyde, formalin and ammonium acetate. MS: 318.2 [M+H]⁺. ¹H NMR (300 MHz, DMSO-d₆) δ 9.08-9.0 (m, 1H), 8.61-8.51 (m, 1H), 8.22 (d, J=8.0 Hz, 1H), 7.80 (s, 1H), 7.69 (dd, J=8.6, 4.2 Hz, 1H), 7.41-7.22 (m, 2H), 6.91 (s, 1H), 4.73-4.58 (m, 1H), 3.67-3.56 (m, 1H), 3.50-3.39 (m, 1H), 3.12 (t, J=11.2 Hz, 1H), 2.57 (t, J=11.5 Hz, 1H), 2.29-2.04 (m, 2H), 1.75-1.57 (m, 1H), 0.99 (d, J=6.3 Hz, 3H).

### Example 159: 5-[(3R,5S)-3-(1H-imidazol-1-yl)-5-methylpiperidin-1-yl]-8-methylquinoline

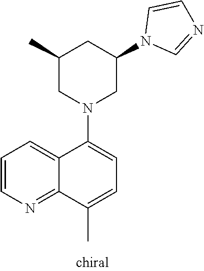 [SMILES: Cc1ccc(N2C[C@@H](C)C[C@@H](n3ccnc3)C2)c2cccnc12]

The title compound was prepared from (3R,5S)-5-methyl-1-(8-methylquinolin-5-yl)piperidin-3-amine, oxaldehyde, formalin and ammonium acetate. MS: 307.0 [M+H]⁺. ¹H NMR (300 MHz, DMSO-d₆) δ 8.94-8.86 (m, 1H), 8.56-8.46 (m, 1H), 7.80 (s, 1H), 7.59-7.44 (m, 2H), 7.32 (s, 1H), 7.11 (d, J=7.6 Hz, 1H), 6.90 (s, 1H), 4.68-4.53 (m, 1H), 3.43-3.34 (m, 1H), 3.25-3.14 (m, 1H), 3.04-2.90 (m, 1H), 2.63 (s, 3H), 2.44-2.30 (m, 1H), 2.24-2.04 (m, 2H), 1.66-1.48 (m, 1H), 0.95 (d, J=6.3 Hz, 3H).

### Example 160: (R)—N-[(3R,5S)-1-(8-Cyano-quinoxalin-5-yl)-5-methyl-piperidin-3-yl]-2,3-dihydroxy-propionamide

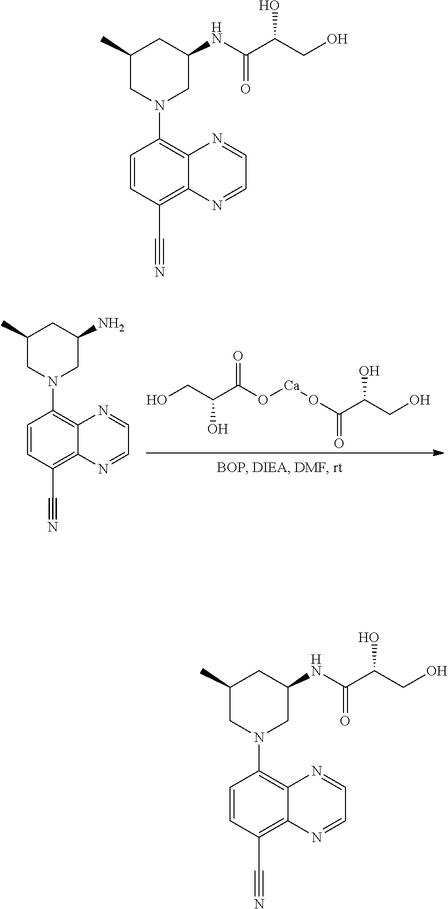

To a mixture of 8-((3R,5S)-3-Amino-5-methyl-piperidin-1-yl)-quinoxaline-5-carbonitrile hydrochloride (2) (45.0 mg; 0.13 mmol; 1.0 eq.), d-glyceric acid calcium salt dihydrate (22.71 mg; 0.08 mmol; 0.60 eq.) and DIEA (65.74 μl; 0.40 mmol; 3.0 eq.) in DMF (2.0 ml; 25.94 mmol; 44.44 V), was added bop (70.19 mg; 0.16 mmol; 1.20 eq.). The resulting mixture was stirred at rt overnight. The crude was purified by prep-HPLC (ACN/water with 0.1% NH₄OH as modifier) to yield the title compound as yellow solid (35.0 mg; 75%). MS: 356 [M+H]⁺. ¹H NMR (400 MHz, DMSO-d₆) δ 9.03 (d, J=1.8 Hz, 1H), 8.94 (d, J=1.8 Hz, 1H), 8.19 (d, J=8.5 Hz, 1H), 7.69 (d, J=8.0 Hz, 1H), 7.27 (d, J=8.5 Hz, 1H), 5.49 (d, J=5.6 Hz, 1H), 4.66 (t, J=5.8 Hz, 1H), 4.28 (d, J=12.1 Hz, 1H), 4.19 (d, J=13.1 Hz, 1H), 4.0-3.84 (m, 2H), 3.59 (ddd, J=11.0, 5.5, 3.6 Hz, 1H), 3.47 (dt, J=11.0, 6.1 Hz, 1H), 2.91 (dd, J=12.2, 10.8 Hz, 1H), 2.73-2.63 (m, 1H), 1.96-1.83 (m, 2H), 1.31 (q, J=12.4 Hz, 1H), 0.92 (d, J=6.3 Hz, 3H).

The following compounds were synthesized in an analogous manner.

### Example 161: (S)—N-[(3R,5S)-1-(8-Cyano-quinoxalin-5-yl)-5-methyl-piperidin-3-yl]-2,3-dihydroxy-3-methyl-butyramide

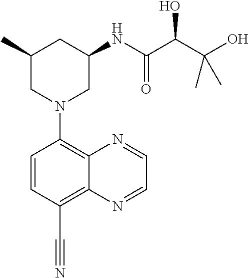 [SMILES: C[C@H]1C[C@@H](NC(=O)[C@@H](O)C(C)(C)O)CN(c2ccc(C#N)c3nccnc23)C1]

The title compound was prepared from 8-((3R,5S)-3-Amino-5-methyl-piperidin-1-yl)-quinoxaline-5-carbonitrile hydrochloride (2) and 2,3-dihydroxyisovaleric acid. The first eluate by prep-HPLC (ACN/water with 0.1% NH₄OH as modifier). MS: 384 [M+H]⁺. ¹H NMR (400 MHz, DMSO-d₆) δ 9.03 (d, J=1.7 Hz, 1H), 8.93 (d, J=1.8 Hz, 1H), 8.20 (d, J=8.4 Hz, 1H), 7.79 (d, J=8.0 Hz, 1H), 7.27 (d, J=8.5 Hz, 1H), 5.52 (d, J=5.8 Hz, 1H), 4.69 (s, 1H), 4.24 (dd, J=34.0, 12.6 Hz, 2H), 4.06-3.93 (m, 1H), 3.64 (d, J=5.8 Hz, 1H), 2.91 (t, J=11.8 Hz, 1H), 2.68 (t, J=11.8 Hz, 1H), 1.97-1.87 (m, 2H), 1.34 (q, J=12.3 Hz, 1H), 1.12 (s, 3H), 1.07 (s, 3H), 0.93 (d, J=6.3 Hz, 3H).

### Example 162: (R)—N-[(3R,5S)-1-(8-Cyano-quinoxalin-5-yl)-5-methyl-piperidin-3-yl]-2,3-dihydroxy-3-methyl-butyramide

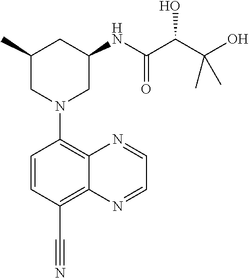 [SMILES: C[C@H]1C[C@@H](NC(=O)[C@H](O)C(C)(C)O)CN(c2ccc(C#N)c3nccnc23)C1]

The title compound was prepared from 8-((3R,5S)-3-Amino-5-methyl-piperidin-1-yl)-quinoxaline-5-carbonitrile hydrochloride (2) and 2,3-dihydroxyisovaleric acid. The second eluate by prep-HPLC (ACN/water with 0.1% NH₄OH as modifier). MS: 384 [M+H]⁺. ¹H NMR (400 MHz, DMSO-d₆) δ 9.03 (d, J=1.8 Hz, 1H), 8.94 (d, J=1.8 Hz, 1H), 8.20 (d, J=8.4 Hz, 1H), 7.80 (d, J=8.2 Hz, 1H), 7.26 (d, J=8.5 Hz, 1H), 5.59 (d, J=5.7 Hz, 1H), 4.69 (s, 1H), 4.28-4.18 (m, 2H), 4.05-3.93 (m, 1H), 3.65 (d, J=5.6 Hz, 1H), 2.96 (t, J=11.5 Hz, 1H), 2.69 (t, J=11.9 Hz, 1H), 1.98-1.86 (m, 2H), 1.31 (q, J=12.3 Hz, 1H), 1.11 (s, 3H), 1.08 (s, 3H), 0.93 (d, J=6.3 Hz, 3H).

### Example 163: (S)-3-Fluoro-pyrrolidine-3-carboxylic acid [(3R,5S)-1-(8-cyano-quinolin-5-yl)-5-trifluoromethyl-piperidin-3-yl]-amide and Example 164: (R)-3-Fluoro-pyrrolidine-3-carboxylic acid [(3R,5S)-1-(8-cyano-quinolin-5-yl)-5-trifluoromethyl-piperidin-3-yl]-amide

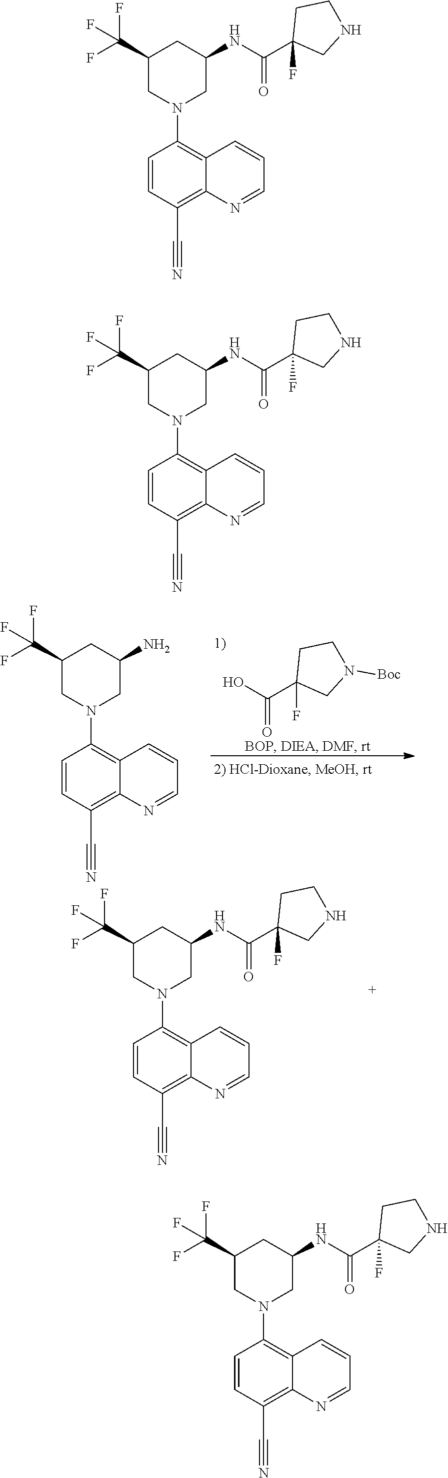

To a mixture of 5-((3R,5S)-3-Amino-5-trifluoromethyl-piperidin-1-yl)-quinoline-8-carbonitrile dihydrochloride (140.0 mg; 0.36 mmol; 1.0 eq.), 3-Fluoro-pyrrolidine-1,3-dicarboxylic acid 1-tert-butyl ester (91.34 mg; 0.39 mmol; 1.10 eq.) and DIEA (176.98 μl; 1.07 mmol; 3.0 eq.) in DMF (1.0 ml; 12.97 mmol; 36.43 eq.), was added bop (188.96 mg; 0.43 mmol; 1.20 eq.). The resulting mixture was stirred at rt 2 h. The reaction mixture was diluted with EtOAc, washed with water (×2) and brine. The organic layer was dried and concentrated to yield the crude 3-[(3R,5 S)-1-(8-Cyano-quinolin-5-yl)-5-trifluoromethyl-piperidin-3-ylcarbamoyl]-3-fluoro-pyrrolidine-1-carboxylic acid tert-butyl ester (190.0 mg; 0.35 mmol), which was used directly for the next step without purification.

To a stirred solution of the crude 3-[(3R,5S)-1-(8-Cyano-quinolin-5-yl)-5-trifluoromethyl-piperidin-3-ylcarbamoyl]-3-fluoro-pyrrolidine-1-carboxylic acid tert-butyl ester (190.0 mg; 0.35 mmol; 1.0 eq.) in methanol (1.90 ml; 10.0 V), was added 4.0M HCl in dioxane (0.89 ml; 3.55 mmol; 10.0 eq.). The resulting mixture was stirred at rt overnight. The reaction mixture was concentrated. The crude was dissolved in DMSO, neutralized to pH-8 and purified by prep-HPLC (ACN/water with 0.1% NH₄OH as modifier).

The first eluate was assigned as Example 163 (65.0 mg; 84%) (Absolute stereochemistry of pyrrolidine ring unknown). MS: 436 [M+H]⁺. ¹H NMR (400 MHz, DMSO-d₆) δ 9.06 (dd, J=4.2, 1.6 Hz, 1H), 8.58 (dd, J=8.6, 1.7 Hz, 1H), 8.39 (dd, J=8.0, 2.7 Hz, 1H), 8.25 (d, J=8.0 Hz, 1H), 7.72 (dd, J=8.6, 4.2 Hz, 1H), 7.34 (d, J=8.1 Hz, 1H), 4.32-4.20 (m, 1H), 3.61-3.47 (m, 2H), 3.29-3.14 (m, 3H), 3.13-2.82 (m, 5H), 2.77 (t, J=11.2 Hz, 1H), 2.25-2.07 (m, 2H), 2.04-1.87 (m, 1H), 1.78 (q, J=12.9, 12.3 Hz, 1H).

The second eluate was assigned as Example 164 (63 mg, 82%) (Absolute stereochemistry of pyrrolidine ring unknown). MS: 436 [M+H]⁺. ¹H NMR (400 MHz, DMSO-d₆) δ 9.06 (dd, J=4.2, 1.6 Hz, 1H), 8.58 (dd, J=8.6, 1.7 Hz, 1H), 8.39 (dd, J=7.9, 2.8 Hz, 1H), 8.25 (d, J=8.0 Hz, 1H), 7.72 (dd, J=8.6, 4.2 Hz, 1H), 7.33 (d, J=8.0 Hz, 1H), 4.32-4.21 (m, 1H), 3.60-3.48 (m, 2H), 3.28-3.16 (m, 3H), 3.10-2.96 (m, 3H), 2.92-2.82 (m, 2H), 2.76 (t, J=11.2 Hz, 1H), 2.29-2.11 (m, 2H), 2.06-1.93 (m, 1H), 1.78 (q, J=12.3 Hz, 1H).

### Example 165: 2-(3-Methyl-3-aza-bicyclo[3.1.1]hept-6-yl)-N-[(3R,5S)-5-methyl-1-(8-methyl-naphthyridin-5-yl)-piperidin-3-yl]-acetamide

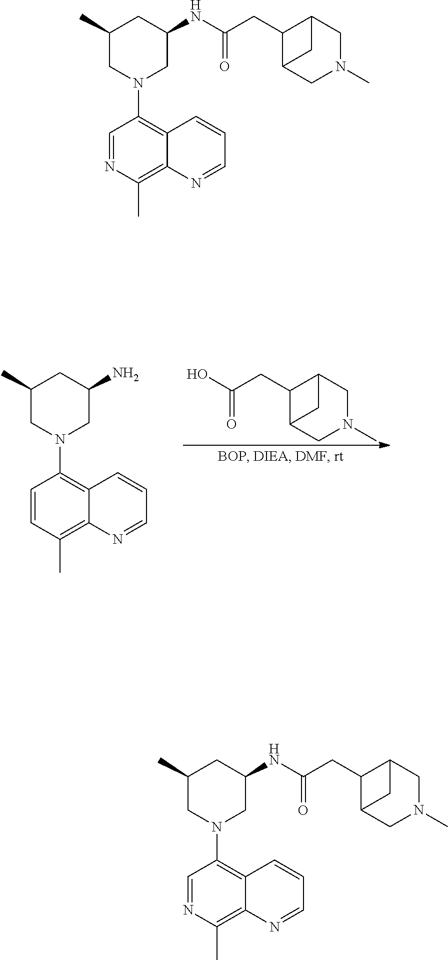

To a mixture of (3R,5S)-5-Methyl-1-(8-methyl-[1,7]naphthyridin-5-yl)-piperidin-3-ylamine dihydrochloride (50.0 mg; 0.15 mmol; 1.0 eq.), (3-Methyl-3-aza-bicyclo[3.1.1]hept-6-yl)-acetic acid (30.59 mg; 0.15 mmol; 1.0 eq.) and DIEA (100.65 μl; 0.61 mmol; 4.0 eq.) in DMF (1.0 ml; 12.97 mmol; 85.41 eq.), was added bop (80.59 mg; 0.18 mmol; 1.20 eq.). The resulting mixture was stirred at rt overnight. The crude was purified by prep-HPLC (ACN/water with 0.10% NH₄OH as modifier to yield the title compound as a yellow solid (50.0 mg; 81%). MS: 408 [M+H]⁺.

### Example 166: 3-Amino-N-[(3R,5S)-5-methyl-1-(8-trifluoromethyl-quinolin-5-yl)-piperidin-3-yl]-propionamide

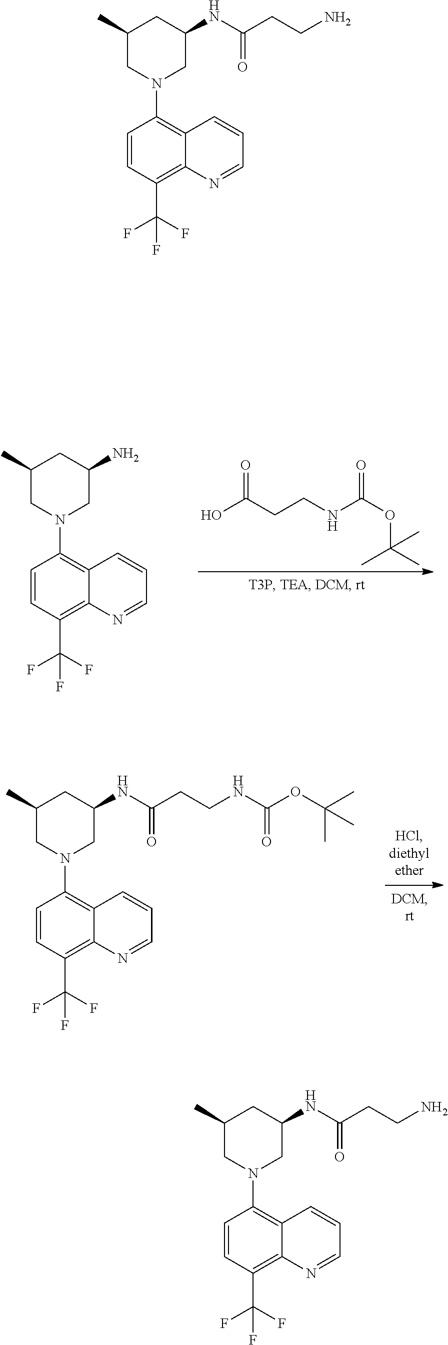

{2-[(3R,5S)-5-Methyl-1-(8-trifluoromethyl-quinolin-5-yl)-piperidin-3-ylcarbamoyl]-ethyl}-carbamic acid tert-butyl ester: In a dry for was suspended 3-(tert-butoxycarbonylamino)propanoic acid (103.51 mg; 0.55 mmol; 1.10 eq.), 1-propanephosphonic anhydride (0.36 ml; 0.60 mmol; 1.20 eq.), and triethylamine (0.24 ml; 1.74 mmol; 3.50 eq.) in dichloromethane (2.0 ml). The reaction mixture was stirred for 15 minutes, then (3R,5S)-5-Methyl-1-(8-trifluoromethyl-quinolin-5-yl)-piperidin-3-ylamine hydrochloride (2) (190.10 mg; 0.50 mmol; 1.0 eq.) was added and the reaction allowed to stir at room temperature for 1 hour. The crude was purified on Biotage using a 15 micron column, with a gradient of 0-20% methanol in dichloromethane to provide the title compound, which was carried forward to Boc-deprotection reaction directly. MS: 481 [M+H]⁺.

3-Amino-N-[(3R,5S)-5-methyl-1-(8-trifluoromethyl-quinolin-5-yl)-piperidin-3-yl]-propionamide: Into a RBF with stirbar was dissolved {2-[(3R,5S)-5-Methyl-1-(8-trifluoromethyl-quinolin-5-yl)-piperidin-3-ylcarbamoyl]-ethyl}-carbamic acid tert-butyl ester (240.30 mg; 0.50 mmol; 1.0 eq.) in minimal dichloromethane. The vial was sealed with a rubber septum, affixed with an Argon inlet, then hydrogen chloride (2M in Ether) (1.25 ml; 2.50 mmol; 5.0 eq.) was added. The reaction was stirred until complete, as determined by LCMS analysis. The crude was purified by prep-HPLC (ACN/water with 0.1% NH₄OH as modifier to yield the title compound (129 mg, 68%). MS:381 [M+H]⁺. ¹H NMR (400 MHz, DMSO-d₆) δ 9.01 (dd, J=4.2, 1.7 Hz, 1H), 8.52 (dd, J=8.6, 1.8 Hz, 1H), 8.04 (d, J=8.1 Hz, 1H), 7.94 (d, J=7.3 Hz, 1H), 7.66 (dd, J=8.6, 4.2 Hz, 1H), 7.20 (d, J=8.1 Hz, 1H), 4.04 (s, 1H), 3.52 (d, J=10.1 Hz, 1H), 3.35 (s, 1H), 2.72 (t, J=6.6 Hz, 2H), 2.43 (q, J=11.4 Hz, 2H), 2.20-2.12 (m, 2H), 1.99 (d, J=13.5 Hz, 1H), 1.09 (q, J=12.2 Hz, 1H), 0.95 (d, J=6.4 Hz, 3H).

The following compounds were synthesized in an analogous manner.

### Example 167: (R)—N-[(3R,5S)-1-(8-Cyano-quinoxalin-5-yl)-5-methyl-piperidin-3-yl]-2,3-dihydroxy-3-methyl-butyramide

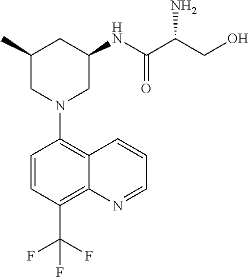 [SMILES: C[C@H]1C[C@@H](NC(=O)[C@H](N)CO)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride (2) and (R)-2-tert-Butoxycarbonylamino-3-hydroxy-propionic acid. MS: 397 [M+H]⁺. ¹H NMR (400 MHz, DMSO-d₆) δ 9.01 (dd, J=4.1, 1.7 Hz, 1H), 8.53 (dd, J=8.6, 1.8 Hz, 1H), 8.05 (d, J=8.1 Hz, 1H), 7.81 (d, J=7.7 Hz, 1H), 7.66 (dd, J=8.6, 4.2 Hz, 1H), 7.19 (d, J=8.1 Hz, 1H), 4.67 (t, J=5.5 Hz, 1H), 4.13-3.97 (m, 1H), 3.48 (p, J=5.3 Hz, 2H), 3.43-3.33 (m, 2H), 3.18 (dd, J=6.3, 4.9 Hz, 1H), 2.43 (t, J=11.3 Hz, 1H), 1.97 (dd, J=48.9, 36.6 Hz, 4H), 1.16 (q, J=12.0 Hz, 1H), 0.95 (d, J=6.6 Hz, 3H).

### Example 168: (S)-3-Hydroxy-2-methylamino-N-[(3R,5S)-5-methyl-1-(8-trifluoromethyl-quinolin-5-yl)-piperidin-3-yl]-propionamide

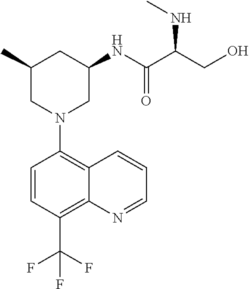 [SMILES: CN[C@@H](CO)C(=O)N[C@@H]1C[C@H](C)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride (2) and methyl (2S)-2-[(tert-butoxycarbonyl)amino]-3-hydroxypropanoate. MS: 411 [M+H]⁺. ¹H NMR (400 MHz, DMSO-d₆) δ 9.01 (dd, J=4.2, 1.7 Hz, 1H), 8.53 (dd, J=8.6, 1.8 Hz, 1H), 8.05 (d, J=8.1 Hz, 1H), 7.78 (d, J=7.8 Hz, 1H), 7.67 (dd, J=8.6, 4.2 Hz, 1H), 7.20 (d, J=8.1 Hz, 1H), 4.67 (t, J=5.6 Hz, 1H), 4.08 (s, 1H), 3.43 (ddt, J=43.3, 10.8, 5.6 Hz, 4H), 2.90 (s, 1H), 2.42 (t, J=11.4 Hz, 1H), 2.22 (s, 3H), 2.15-1.85 (m, 3H), 1.18 (q, J=12.9, 12.1 Hz, 2H), 0.95 (d, J=6.6 Hz, 3H).

### Example 169: (2S,3R)-2-Amino-3-hydroxy-N-[(3R,5S)-5-methyl-1-(8-trifluoromethyl-quinolin-5-yl)-piperidin-3-yl]-butyramide

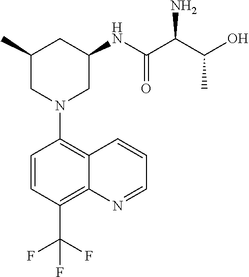 [SMILES: C[C@H]1C[C@@H](NC(=O)[C@@H](N)[C@@H](C)O)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride (2) and (2S,3R)-2-tert-butoxycarbonylamino-3-hydroxy-butyric acid. MS:411 [M+H]⁺. ¹H NMR (400 MHz, DMSO-d₆) δ 9.01 (dd, J=4.2, 1.7 Hz, 1H), 8.53 (dd, J=8.6, 1.8 Hz, 1H), 8.05 (d, J=8.1 Hz, 1H), 7.84 (d, J=7.7 Hz, 1H), 7.67 (dd, J=8.6, 4.2 Hz, 1H), 7.19 (d, J=8.0 Hz, 1H), 4.55 (d, J=4.9 Hz, 1H), 4.05 (d, J=11.3 Hz, 1H), 3.74 (p, J=6.2, 5.8 Hz, 1H), 3.49 (d, J=10.5 Hz, 1H), 2.89 (d, J=4.8 Hz, 1H), 2.43 (t, J=11.4 Hz, 1H), 2.17-1.92 (m, 2H), 1.74 (s, 2H), 1.15 (q, J=12.0 Hz, 1H), 1.04 (d, J=6.3 Hz, 3H), 0.95 (d, J=6.6 Hz, 3H).

### Example 170: (S)-2-Amino-3-hydroxy-3-methyl-N-[(3R,5S)-5-methyl-1-(8-trifluoromethyl-quinolin-5-yl)-piperidin-3-yl]-butyramide

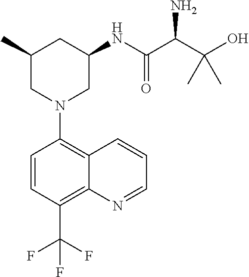 [SMILES: C[C@H]1C[C@@H](NC(=O)[C@@H](N)C(C)(C)O)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride (2) and (S)-2-tert-butoxycarbonylamino-3-hydroxy-3-methyl-butyric acid. MS:425 [M+H]⁺. ¹H NMR (400 MHz, DMSO-d₆) δ 9.01 (dd, J=4.2, 1.7 Hz, 1H), 8.53 (dd, J=8.6, 1.8 Hz, 1H), 8.06 (d, J=8.1 Hz, 1H), 7.85 (d, J=7.6 Hz, 1H), 7.67 (dd, J=8.6, 4.2 Hz, 1H), 7.20 (d, J=8.1 Hz, 1H), 4.55 (s, 1H), 4.08 (d, J=10.0 Hz, 1H), 3.48 (d, J=9.8 Hz, 1H), 3.0 (s, 1H), 2.43 (t, J=11.3 Hz, 1H), 2.15-1.93 (m, 2H), 1.77 (s, 2H), 1.07 (d, J=14.0 Hz, 7H), 0.95 (d, J=6.5 Hz, 3H).

### Example 171: (S)-2-Amino-N-[(3R,5S)-5-trifluoromethyl-1-(8-trifluoromethyl-quinolin-5-yl)-piperidin-3-yl]-propionamide

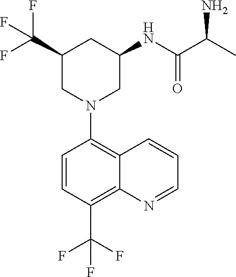 [SMILES: C[C@H](N)C(=O)N[C@@H]1C[C@H](C(F)(F)F)CN(c2ccc(C(F)(F)F)c3ncccc23)C1]

The title compound was prepared from (3R,5S)-5-Trifluoromethyl-1-(8-trifluoromethyl-quinolin-5-yl)-piperidin-3-ylamine hydrochloride (2) and (S)-2-(N-tert-Butoxycarbonyl)aminopropionic acid, followed by chiral separation by SFC using a Cel-4 column, with an isocratic gradient of 21% 0.5% DMEA in methanol solution over CO2 and a flow rate of 70 g/min. MS:435 [M+H]⁺. ¹H NMR (400 MHz, DMSO-d₆) δ 9.04 (dd, J=4.2, 1.7 Hz, 1H), 8.60 (dt, J=8.7, 1.3 Hz, 1H), 8.08 (d, J=8.1 Hz, 1H), 7.70 (ddd, J=8.6, 4.2, 1.4 Hz, 1H), 7.32 (d, J=8.0 Hz, 1H), 3.49 (dd, J=18.0, 11.8 Hz, 2H), 3.28-3.12 (m, 4H), 2.87 (t, J=11.5 Hz, 1H), 2.65 (t, J=11.1 Hz, 1H), 2.17 (d, J=12.1 Hz, 2H), 2.08 (s, 1H), 1.12 (d, J=6.9 Hz, 3H).

### Example 172: (R)-2-Amino-3-hydroxy-N-[(3R,5S)-5-trifluoromethyl-1-(8-trifluoromethyl-quinolin-5-yl)-piperidin-3-yl]-propionamide

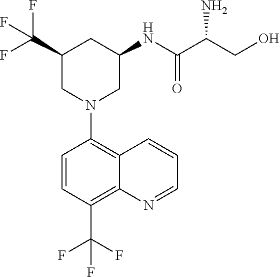 [SMILES: N[C@H](CO)C(=O)N[C@@H]1C[C@H](C(F)(F)F)CN(c2ccc(C(F)(F)F)c3ncccc23)C1]

The title compound was prepared from (3R,5S)-5-Trifluoromethyl-1-(8-trifluoromethyl-quinolin-5-yl)-piperidin-3-ylamine hydrochloride (2) and (R)-2-(tert-Butoxycarbonylamino)-3-hydroxypropanoic acid, followed by chiral separation by SFC using an IC column, with an isocratic gradient of 10% 0.5% DMEA in methanol solution over CO2 and a flow rate of 70 g/min. MS:451 [M+H]⁺. ¹H NMR (400 MHz, DMSO-d₆) δ 9.03 (dd, J=4.2, 1.7 Hz, 1H), 8.59 (dd, J=8.6, 1.8 Hz, 1H), 8.08 (d, J=8.1 Hz, 1H), 7.97 (d, J=7.8 Hz, 1H), 7.70 (dd, J=8.6, 4.2 Hz, 1H), 7.31 (d, J=8.0 Hz, 1H), 4.67 (t, J=5.6 Hz, 1H), 4.17 (s, 1H), 3.57-3.34 (m, 4H), 3.18 (t, J=5.5 Hz, 2H), 2.87 (t, J=11.4 Hz, 1H), 2.66 (t, J=11.0 Hz, 1H), 2.16 (d, J=11.8 Hz, 1H), 1.77 (s, 2H), 1.61 (q, J=12.2 Hz, 1H).

### Example 173: (S)-2-Amino-N-[(3R,5S)-1-(8-cyano-quinoxalin-5-yl)-5-methyl-piperidin-3-yl]-3-hydroxy-3-methyl-butyramide

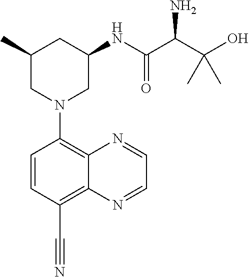 [SMILES: C[C@H]1C[C@@H](NC(=O)[C@@H](N)C(C)(C)O)CN(c2ccc(C#N)c3nccnc23)C1]

The title compound was prepared from 8-((3R,5S)-3-Amino-5-methyl-piperidin-1-yl)-quinoxaline-5-carbonitrile hydrochloride (3) and (S)-2-(tert-Butoxycarbonylamino)-3-hydroxy-3-methylbutanoic acid. MS: 383 [M+H]⁺. ¹H NMR (400 MHz, DMSO-d₆) δ 9.03 (d, J=1.8 Hz, 1H), 8.94 (d, J=1.8 Hz, 1H), 8.20 (d, J=8.4 Hz, 1H), 7.87 (d, J=7.7 Hz, 1H), 7.27 (d, J=8.5 Hz, 1H), 4.56 (s, 1H), 4.27 (s, 2H), 4.02-3.84 (m, 1H), 3.01 (s, 1H), 2.82 (t, J=11.5 Hz, 1H), 2.76-2.62 (m, 1H), 2.03-1.68 (m, 4H), 1.20 (q, J=11.7 Hz, 1H), 1.08 (d, J=13.2 Hz, 6H), 0.92 (d, J=6.5 Hz, 3H).

### Example 174: (R)-Pyrrolidine-2-carboxylic acid [(3R,5S)-1-(8-cyano-quinoxalin-5-yl)-5-methyl-piperidin-3-yl]-amide

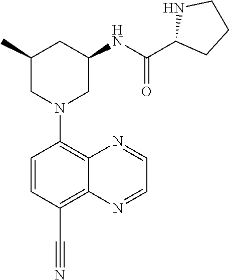 [SMILES: C[C@H]1C[C@@H](NC(=O)[C@H]2CCCN2)CN(c2ccc(C#N)c3nccnc23)C1]

The title compound was prepared from 8-((3R,5S)-3-Amino-5-methyl-piperidin-1-yl)-quinoxaline-5-carbonitrile hydrochloride (3) and (2R)-1-tert-butoxycarbonylpyrrolidine-2-carboxylic acid. MS: 365 [M+H]⁺. ¹H NMR (400 MHz, DMSO-d₆) δ 9.02 (d, J=1.8 Hz, 1H), 8.94 (d, J=1.8 Hz, 1H), 8.18 (d, J=8.4 Hz, 1H), 7.90 (d, J=8.1 Hz, 1H), 7.27 (d, J=8.5 Hz, 1H), 4.23 (dd, J=33.3, 12.4 Hz, 2H), 3.87 (s, 0H), 3.51 (dd, J=8.8, 5.4 Hz, 1H), 2.91-2.73 (m, 4H), 2.73-2.62 (m, 1H), 2.01-1.82 (m, 4H), 1.73-1.51 (m, 4H), 1.25 (q, J=12.5 Hz, 2H), 0.92 (d, J=6.3 Hz, 3H).

### Example 175: (S)-Pyrrolidine-2-carboxylic acid [(3R,5S)-1-(8-cyano-quinoxalin-5-yl)-5-methyl-piperidin-3-yl]-amide

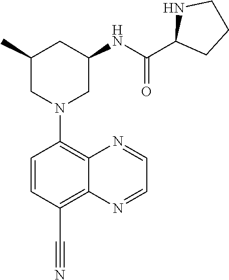 [SMILES: C[C@H]1C[C@@H](NC(=O)[C@@H]2CCCN2)CN(c2ccc(C#N)c3nccnc23)C1]

The title compound was prepared from 8-((3R,5S)-3-Amino-5-methyl-piperidin-1-yl)-quinoxaline-5-carbonitrile hydrochloride (3) and (2S)-1-tert-butoxycarbonylpyrrolidine-2-carboxylic acid. MS: 365 [M+H]⁺. ¹H NMR (400 MHz, DMSO-d₆) δ 9.02 (d, J=1.8 Hz, 1H), 8.94 (d, J=1.8 Hz, 1H), 8.19 (d, J=8.4 Hz, 1H), 7.89 (d, J=8.0 Hz, 1H), 7.28 (d, J=8.5 Hz, 1H), 4.32 (d, J=11.8 Hz, 1H), 3.97-3.80 (m, 1H), 3.48 (dd, J=8.7, 5.4 Hz, 1H), 2.90-2.75 (m, 4H), 2.68 (dd, J=12.7, 11.0 Hz, 1H), 2.0-1.83 (m, 3H), 1.71-1.53 (m, 3H), 1.25 (q, J=12.0 Hz, 1H), 0.91 (d, J=6.4 Hz, 3H).

### Example 176: (S)-Pyrrolidine-2-carboxylic acid [(3R,5S)-5-methyl-1-(8-trifluoromethyl-quinolin-5-yl)-piperidin-3-yl]-amide

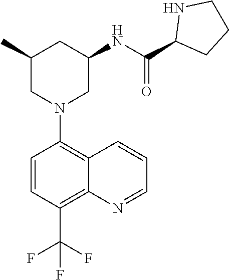 [SMILES: C[C@H]1C[C@@H](NC(=O)[C@@H]2CCCN2)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride (2) and (2S)-1-tert-butoxycarbonylpyrrolidine-2-carboxylic acid. MS: 407 [M+H]⁺. ¹H NMR (400 MHz, DMSO-d₆) δ 9.01 (dd, J=4.1, 1.7 Hz, 1H), 8.52 (dd, J=8.6, 1.8 Hz, 1H), 8.04 (d, J=8.1 Hz, 1H), 7.86 (d, J=7.9 Hz, 1H), 7.66 (dd, J=8.6, 4.2 Hz, 1H), 7.20 (d, J=8.0 Hz, 1H), 4.02 (ddt, J=15.9, 11.7, 6.0 Hz, 1H), 3.47 (td, J=11.4, 10.0, 6.5 Hz, 2H), 3.34 (d, J=3.8 Hz, 1H), 2.79 (td, J=6.4, 1.8 Hz, 2H), 2.55 (d, J=10.9 Hz, 1H), 2.42 (t, J=11.4 Hz, 1H), 2.17-1.86 (m, 2H), 1.68-1.50 (m, 3H), 1.29-1.10 (m, 1H), 0.94 (d, J=6.6 Hz, 3H).

### Example 177: (R)-Pyrrolidine-2-carboxylic acid [(3R,5S)-5-methyl-1-(8-trifluoromethyl-quinolin-5-yl)-piperidin-3-yl]-amide

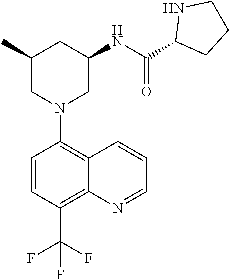 [SMILES: C[C@H]1C[C@@H](NC(=O)[C@H]2CCCN2)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride (2) and (2R)-1-tert-butoxycarbonylpyrrolidine-2-carboxylic acid. MS: 407 [M+H]⁺. ¹H NMR (400 MHz, DMSO-d₆) δ 9.0 (dd, J=4.2, 1.7 Hz, 1H), 8.52 (dd, J=8.6, 1.8 Hz, 1H), 8.04 (d, J=8.1 Hz, 1H), 7.86 (d, J=8.0 Hz, 1H), 7.66 (dd, J=8.6, 4.2 Hz, 1H), 7.20 (d, J=8.1 Hz, 1H), 4.10-3.95 (m, 1H), 3.54-3.40 (m, 2H), 3.34 (s, 1H), 2.87-2.71 (m, 2H), 2.56 (t, J=10.9 Hz, 1H), 2.42 (t, J=11.4 Hz, 1H), 2.15-1.85 (m, 3H), 1.72-1.52 (m, 3H), 1.17 (q, J=12.0 Hz, 1H), 0.94 (d, J=6.6 Hz, 3H).

### Example 178: (S)-2-Amino-3-hydroxy-2-methyl-N-[(3R,5S)-5-methyl-1-(8-trifluoromethyl-quinolin-5-yl)-piperidin-3-yl]-propionamide

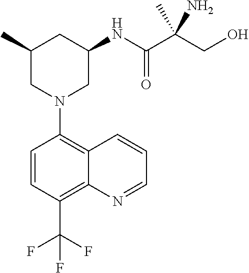 [SMILES: C[C@H]1C[C@@H](NC(=O)[C@@](C)(N)CO)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride (2) and (S)-2-(tert-Butoxycarbonylamino)-3-hydroxy-2-methylpropanoic acid. MS: 411 [M+H]⁺. ¹H NMR (400 MHz, DMSO-d₆) δ 9.01 (dd, J=4.2, 1.7 Hz, 1H), 8.53 (dd, J=8.6, 1.8 Hz, 1H), 8.05 (d, J=8.1 Hz, 1H), 7.77 (d, J=7.9 Hz, 1H), 7.67 (dd, J=8.6, 4.2 Hz, 1H), 7.19 (d, J=8.0 Hz, 1H), 4.74 (t, J=5.5 Hz, 1H), 3.57 (dd, J=10.1, 5.8 Hz, 1H), 3.47 (d, J=9.2 Hz, 1H), 3.11 (dd, J=10.1, 5.3 Hz, 1H), 2.55 (d, J=10.9 Hz, 2H), 2.43 (t, J=11.3 Hz, 1H), 2.16-1.76 (m, 4H), 1.19 (q, J=11.9 Hz, 1H), 1.06 (s, 3H), 0.95 (d, J=6.6 Hz, 3H).

### Example 179: Piperidine-4-carboxylic acid [(3R,5S)-5-methyl-1-(8-trifluoromethyl-quinolin-5-yl)-piperidin-3-yl]-amide

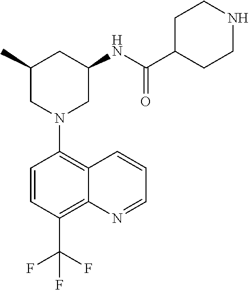 [SMILES: C[C@H]1C[C@@H](NC(=O)C2CCNCC2)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride (2) and 1-(tert-butoxycarbonyl)piperidine-4-carboxylic acid. MS: 421 [M+H]⁺. ¹H NMR (400 MHz, DMSO-d₆) δ 9.01 (dd, J=4.2, 1.7 Hz, 1H), 8.52 (dt, J=8.6, 1.7 Hz, 1H), 8.05 (d, J=8.1 Hz, 1H), 7.66 (dd, J=8.5, 4.1 Hz, 1H), 7.58 (dd, J=10.0, 7.8 Hz, 1H), 7.19 (dd, J=8.1, 3.7 Hz, 1H), 3.46 (d, J=11.4 Hz, 1H), 3.34 (s, 1H), 3.01 (d, J=9.0 Hz, 1H), 2.89 (d, J=12.5 Hz, 1H), 2.41 (t, J=11.4 Hz, 1H), 2.17-1.89 (m, 3H), 1.70 (d, J=9.9 Hz, 2H), 1.49-1.08 (m, 5H), 0.94 (dd, J=6.6, 1.3 Hz, 3H).

### Example 180: 3-Amino-piperidine-3-carboxylic acid [(3R,5S)-5-methyl-1-(8-trifluoromethyl-quinolin-5-yl)-piperidin-3-yl]-amide

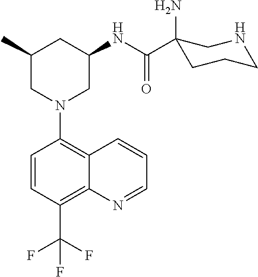 [SMILES: C[C@H]1C[C@@H](NC(=O)C2(N)CCCNC2)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride (2) and 3-Amino-1-(tert-butoxycarbonyl)piperidine-3-carboxylic acid. MS: 436 [M+H]⁺. ¹H NMR (400 MHz, DMSO-d₆) δ 9.01 (dd, J=4.2, 1.7 Hz, 1H), 8.53 (dd, J=8.6, 1.8 Hz, 1H), 8.23 (d, J=31.8 Hz, 2H), 8.05 (d, J=8.0 Hz, 1H), 7.67 (dd, J=8.6, 4.2 Hz, 1H), 7.20 (d, J=8.1 Hz, 1H), 3.34 (d, J=12.0 Hz, 2H), 2.98 (d, J=10.1 Hz, 1H), 2.79 (s, 1H), 2.75-2.53 (m, 3H), 2.44 (td, J=11.2, 3.1 Hz, 1H), 2.16-1.93 (m, 2H), 1.86 (q, J=9.3 Hz, 1H), 1.69 (s, 1H), 1.44 (d, J=11.2 Hz, 2H), 1.27-1.10 (m, 1H), 0.96 (d, J=6.5 Hz, 3H).

### Example 181: Piperidine-2-carboxylic acid [(3R,5S)-5-methyl-1-(8-trifluoromethyl-quinolin-5-yl)-piperidin-3-yl]-amide

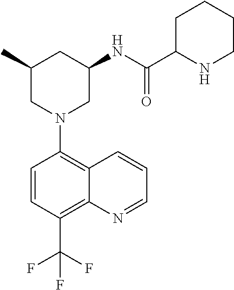 [SMILES: C[C@H]1C[C@@H](NC(=O)C2CCCCN2)CN(c2ccc(C(F)(F)F)c3ncccc23)C1]

The title compound was prepared from (3R,5S)-5-Methyl-1-(8-trifluoromethyl-quinolin-5-yl)-piperidin-3-ylamine hydrochloride (2) and 1-tert-butoxycarbonyl-piperidine-2-carboxylic acid. MS: 421 [M+H]⁺. ¹H NMR (400 MHz, DMSO-d₆) δ 9.0 (dd, J=4.2, 1.7 Hz, 1H), 8.52 (dd, J=8.6, 1.8 Hz, 1H), 8.04 (d, J=8.1 Hz, 1H), 7.80-7.60 (m, 2H), 7.19 (d, J=8.0 Hz, 1H), 4.08-3.91 (m, 1H), 3.48 (dd, J=11.3, 4.0 Hz, 1H), 3.0-2.82 (m, 2H), 2.41 (tdd, J=15.5, 9.9, 5.5 Hz, 4H), 2.23-1.89 (m, 3H), 1.62-1.32 (m, 4H), 1.09 (q, J=12.1 Hz, 1H), 0.94 (d, J=6.5 Hz, 3H).

### Example 182: 1-((2S,3R)-2-Amino-3-hydroxy-butyryl)-piperidine-4-carboxylic acid [(3R,5S)-5-methyl-1-(8-trifluoromethyl-quinolin-5-yl)-piperidin-3-yl]-amide

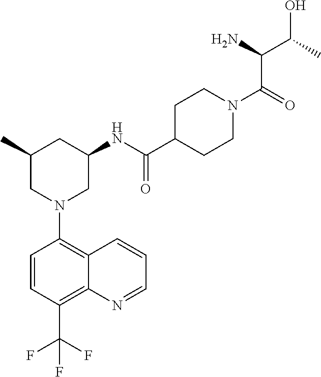 [SMILES: C[C@H]1C[C@@H](NC(=O)C2CCN(C(=O)[C@@H](N)[C@@H](C)O)CC2)CN(c2ccc(C(F)(F)F)c3ncccc23)C1]

The title compound was prepared from Piperidine-4-carboxylic acid [(3R,5S)-5-methyl-1-(8-trifluoromethyl-quinolin-5-yl)-piperidin-3-yl]-amide and (2S,3R)-2-tert-Butoxycarbonylamino-3-hydroxy-butyric acid. MS: 522 [M+H]⁺. ¹H NMR (400 MHz, DMSO-d₆) δ 9.01 (dd, J=4.2, 1.7 Hz, 1H), 8.53 (dd, J=8.6, 1.8 Hz, 1H), 8.05 (d, J=8.1 Hz, 1H), 7.71-7.59 (m, 2H), 7.21 (d, J=8.1 Hz, 1H), 5.14 (dd, J=13.1, 4.7 Hz, 1H), 4.17-3.82 (m, 3H), 3.81-3.40 (m, 3H), 3.04 (t, J=12.5 Hz, 1H), 2.41 (q, J=11.4, 10.8 Hz, 1H), 2.29-1.84 (m, 4H), 1.84-1.07 (m, 9H), 1.07-0.85 (m, 6H).

### Example 183: Piperidine-2-carboxylic acid [(3R,5S)-1-(8-cyano-quinolin-5-yl)-5-trifluoromethyl-piperidin-3-yl]-amide

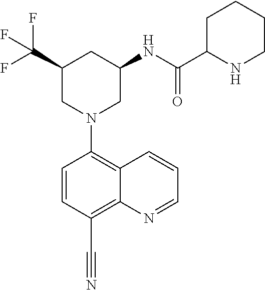 [SMILES: N#Cc1ccc(N2C[C@H](NC(=O)C3CCCCN3)C[C@H](C(F)(F)F)C2)c2cccnc12]

The title compound was prepared from 5-((3R,5S)-3-Amino-5-trifluoromethyl-piperidin-1-yl)-quinoline-8-carbonitrile hydrochloride (3) and 1-tert-butoxycarbonyl-piperidine-2-carboxylic acid. MS: 431 [M+H]⁺. ¹H NMR (400 MHz, DMSO-d₆) δ 9.05 (dd, J=4.3, 1.6 Hz, 1H), 8.57 (dt, J=8.6, 1.9 Hz, 1H), 8.25 (d, J=8.0 Hz, 1H), 7.78 (dd, J=7.9, 5.4 Hz, 1H), 7.71 (ddd, J=8.6, 4.2, 0.8 Hz, 1H), 7.31 (dd, J=8.1, 3.8 Hz, 1H), 4.17 (s, 1H), 3.52 (dd, J=27.6, 9.9 Hz, 2H), 3.19 (s, 1H), 3.02 (d, J=9.8 Hz, 1H), 2.91 (t, J=11.8 Hz, 2H), 2.77-2.62 (m, 1H), 2.26-2.04 (m, 2H), 1.66 (dt, J=24.5, 11.7 Hz, 3H), 1.45 (d, J=11.6 Hz, 1H), 1.39-1.15 (m, 3H).

### Example 184: 2-[1-((2S,3R)-2-Amino-3-hydroxy-butyryl)-piperidin-4-yl]-N-[(3R,5S)-1-(8-cyano-quinolin-5-yl)-5-trifluoromethyl-piperidin-3-yl]-acetamide

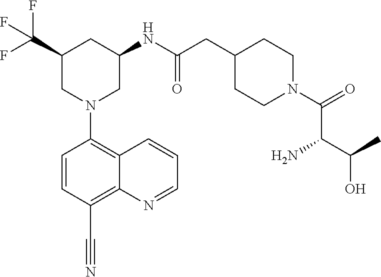 [SMILES: C[C@@H](O)[C@H](N)C(=O)N1CCC(CC(=O)N[C@@H]2C[C@H](C(F)(F)F)CN(c3ccc(C#N)c4ncccc34)C2)CC1]

The title compound was prepared from N-[(3R,5S)-1-(8-Cyanoquinolin-5-yl)-5-trifluoromethyl-piperidin-3-yl]-2-piperidin-4-yl-acetamide hydrochloride (3) and (2S,3R)-2-tert-Butoxycarbonylamino-3-hydroxy-butyric acid. MS:547 [M+H]⁺. ¹H NMR (400 MHz, DMSO-d₆) δ 9.06 (dd, J=4.2, 1.6 Hz, 1H), 8.58 (dd, J=8.6, 1.7 Hz, 1H), 8.25 (d, J=8.0 Hz, 1H), 8.02 (d, J=7.3 Hz, 1H), 7.71 (dd, J=8.6, 4.2 Hz, 1H), 7.32 (d, J=8.1 Hz, 1H), 4.54 (s, 1H), 4.35 (s, 1H), 4.16 (s, 1H), 3.98 (s, 1H), 3.62-3.50 (m, 3H), 3.44 (s, 1H), 2.94 (t, J=11.6 Hz, 2H), 2.60 (t, J=11.3 Hz, 1H), 2.18 (d, J=12.1 Hz, 1H), 2.11-1.81 (m, 4H), 1.64 (d, J=13.4 Hz, 2H), 1.50 (q, J=12.3 Hz, 1H), 0.99 (d, J=6.2 Hz, 5H).

### Example 185: Piperidine-3-carboxylic acid [(3R,5S)-1-(8-cyano-quinolin-5-yl)-5-trifluoromethyl-piperidin-3-yl]-amide

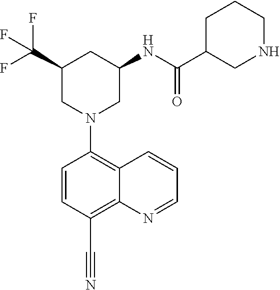 [SMILES: N#Cc1ccc(N2C[C@H](NC(=O)C3CCCNC3)C[C@H](C(F)(F)F)C2)c2cccnc12]

The title compound was prepared from 5-((3R,5S)-3-Amino-5-trifluoromethyl-piperidin-1-yl)-quinoline-8-carbonitrile hydrochloride (3) and 3-Carbamoyl-piperidine-1-carboxylic acid tert-butyl ester. MS: 432 [M+H]⁺. ¹H NMR (400 MHz, DMSO-d₆) δ 9.05 (dd, J=4.2, 1.6 Hz, 1H), 8.57 (dd, J=8.6, 1.7 Hz, 1H), 8.24 (d, J=8.0 Hz, 1H), 7.98 (d, J=7.3 Hz, 1H), 7.71 (dd, J=8.6, 4.2 Hz, 1H), 7.31 (d, J=8.1 Hz, 1H), 4.12 (s, 1H), 3.63-3.47 (m, 2H), 2.98-2.72 (m, 3H), 2.64-2.53 (m, 1H), 2.39 (t, J=11.9 Hz, 1H), 2.17 (dd, J=11.8, 9.1 Hz, 2H), 1.71 (d, J=12.7 Hz, 1H), 1.59-1.40 (m, 3H), 1.37-1.19 (m, 1H).

### Example 186: 3-Dimethylamino-N-[(3R,5S)-5-methyl-1-(8-trifluoromethyl-quinolin-5-yl)-piperidin-3-yl]-propionamide

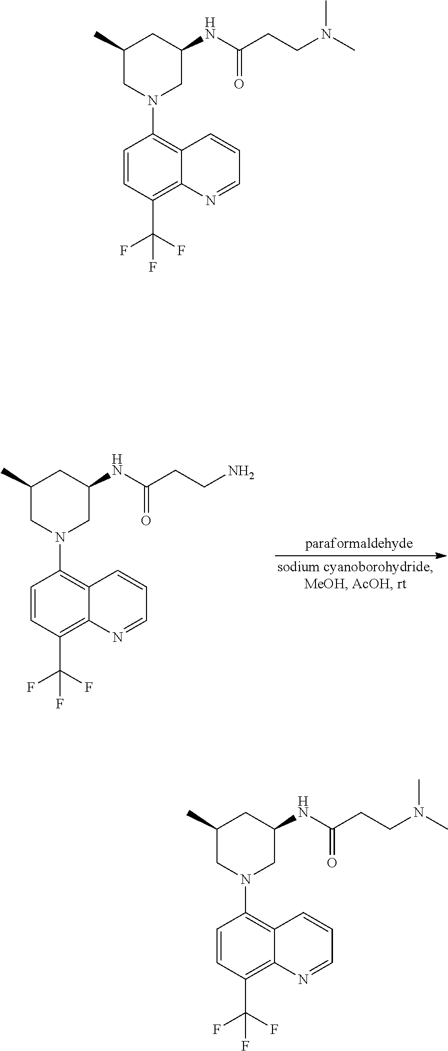

{2-[(3R,5S)-5-Methyl-1-(8-trifluoromethyl-quinolin-5-yl)-piperidin-3-ylcarbamoyl]-ethyl}-carbamic acid tert-butyl ester: A solution of the 3-amino-N-[(3R,5S)-5-methyl-1-(8-trifluoromethyl-quinolin-5-yl)-piperidin-3-yl]-propionamide hydrochloride (3) (44.3 mg; 0.09 mmol; 1.0 eq.), paraformaldehyde (8.1 mg; 0.09 mmol; 1.0 eq.) and sodium cyanoborohydride (1.0M in THF) (0.11 ml; 0.11 mmol; 1.2 eq.) in methanol (1 ml) and Acetic acid (0.05 ml) was stirred under Ar at ambient temperature for 8-10 h. The desired product was isolated by Prep HPLC under basic conditions as a fluffy white solid. MS: 409 [M+H]⁺. ¹H NMR (400 MHz, DMSO-d₆) δ 9.01 (dd, J=4.2, 1.7 Hz, 1H), 8.53 (dd, J=8.6, 1.8 Hz, 1H), 8.05 (d, J=8.1 Hz, 1H), 7.96 (d, J=7.4 Hz, 1H), 7.67 (dd, J=8.6, 4.2 Hz, 1H), 7.20 (d, J=8.1 Hz, 1H), 4.02 (s, 1H), 3.52 (d, J=9.7 Hz, 1H), 2.43 (tdd, J=11.5, 7.1, 4.4 Hz, 4H), 2.21 (td, J=6.9, 3.3 Hz, 2H), 2.11 (s, 7H), 1.99 (d, J=12.9 Hz, 1H), 1.08 (q, J=12.0 Hz, 1H), 0.95 (d, J=6.5 Hz, 3H).

### Example 187: 8-((3R,5S)-3-Amino-5-methyl-piperidin-1-yl)-pyrido[3,4-b]pyrazine-5-carbonitrile hydrochloride

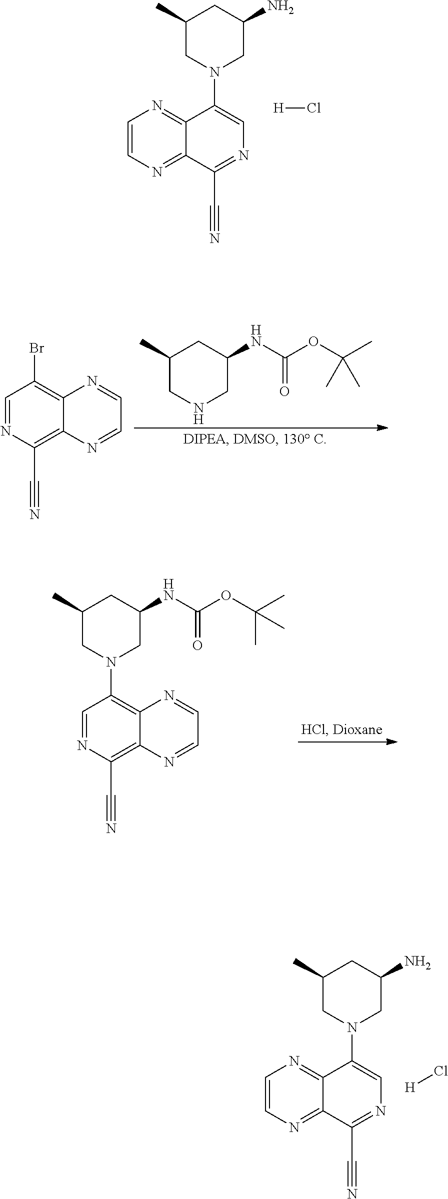

[(3R,5S)-1-(5-Cyano-pyrido[3,4-b]pyrazin-8-yl)-5-methyl-piperidin-3-yl]-carbamic acid tert-butyl ester: In a 10 mL microwave vial, 8-Bromo-pyrido[3,4-b]pyrazine-5-carbonitrile (80.0 mg; 0.298 mmol), ((3R,5S)-5-Methyl-piperidin-3-yl)-carbamic acid tert-butyl ester (95.8 mg; 0.447 mmol) and DIPEA (155.2 μl; 0.894 mmol) were dissolved in anhydrous DMSO (2.0 ml). The tube was sealed and flushed with nitrogen for 10 min and the brown suspension was microwaved at 130° C. for 3 h. The brown solution was poured over water (50 mL) and extracted with ethyl acetate (3×25 mL). The combined organic phase was washed with brine (50 mL), dried over anhydrous sodium sulfate, filter and concentrated under reduced pressure. The residue was dissolved in DCM and MeOH, adsorb on a PuriFlash 4 g 30u column and purified by chromatography on a PuriFlash 12 g 30u column (Hexane-AcOEt 10% for 5 column volumes, Hexane-AcOEt 10-60% for 15 column volumes), major product eluted with AcOEt 43-49% (lambda max 278 nm). The pure fractions were concentrated under reduced pressure and the solid was dried under vacuo to yield the title compound as an orange solid (108.0 mg; 98.3%). MS: 369 [M+H]⁺.

8-((3R,5S)-3-Amino-5-methyl-piperidin-1-yl)-pyrido[3,4-b]pyrazine-5-carbonitrile hydrochloride: In a 100 mL round bottom flask, [(3R,5S)-1-(5-Cyano-pyrido[3,4-b]pyrazin-8-yl)-5-methyl-piperidin-3-yl]-carbamic acid tert-butyl ester (85.0 mg; 0.231 mmol) was dissolved in anhydrous methanol (3.0 ml). A solution of hydrochloric acid (1.7 ml; 6.921 mmol, 4M in dioxane) was added and the orange solution was stirred at room temperature overnight. Ether (10 mL) was added to the light orange solution and the peach suspension was stirred at room temperature for 1 h. The peach solid was filtered, washed with ether and dried under vacuo to yield the title compound as a yellow solid (77.0 mg; 109.5%). MS: 269 [M+H]⁺. ¹H NMR (400 MHz, Deuterium Oxide) d 9.15 (d, J=1.9 Hz, 1H), 9.10 (d, J=1.9 Hz, 1H), 8.45 (s, 1H), 4.70 (d, J=12.6 Hz, 1H), 4.14 (d, J=12.4 Hz, 1H), 3.76 (qd, J=9.9, 8.3, 4.0 Hz, 1H), 3.24 (t, J=11.6 Hz, 1H), 2.89 (t, J=12.0 Hz, 1H), 2.37 (d, J=12.4 Hz, 1H), 2.24-2.10 (m, 1H), 1.44 (q, J=12.0 Hz, 1H), 1.10 (d, J=6.5 Hz, 3H).

### Example 188: N-[(3R,5S)-1-(5-Cyano-pyrido[3,4-b]pyrazin-8-yl)-5-methyl-piperidin-3-yl]-2-(4-methyl-piperazin-1-yl)-acetamide

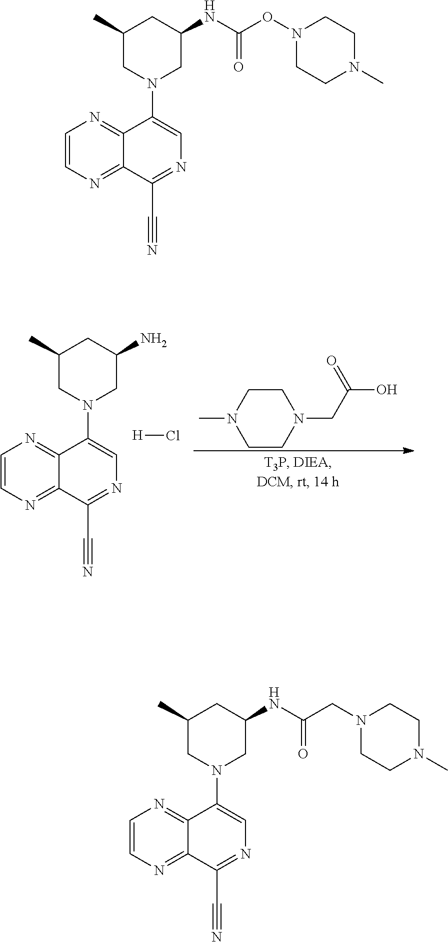

In a 20 mL scintillation vial, 8-((3R,5S)-3-Amino-5-methyl-piperidin-1-yl)-pyrido[3,4-b]pyrazine-5-carbonitrile hydrochloride (60.0 mg; 0.197 mmol), (4-methyl-piperazin-1-yl)-acetic acid (62.3 mg; 0.394 mmol) and DIPEA (171.5 μl; 0.984 mmol) were dissolved in anhydrous DCM (3.0 ml). A solution of 50% 2,4,6-tripropyl-1,3,5,2,4,6-trioxatriphosphorinane-2,4,6-trioxide (347.4 μl; 0.591 mmol) in ethyl acetate was added and the orange solution was stirred overnight at room temperature. The yellow solution was concentrated under reduced pressure. The residue was dissolved in DCM, absorbed on a PuriFlash 6 g 50u NH₂ column and purified by chromatography on a PuriFlash 35 g 30u NH₂ column (AcOEt-DCM 40% for 5 column volumes, AcOEt-DCM 40-100% for 10 column volumes), major product eluted with DCM 50-87% (lambda max 280) The pure fractions were concentrated under reduced pressure, the solid was dissolved in acetonitrile and water and lyophilized to provide the title compound as an orange solid (40.0 mg; 49.7%). MS:409 [M+H]⁺. H NMR (400 MHz, Chloroform-d) δ 9.02 (d, J=1.6 Hz, 1H), 8.97 (d, J=1.6 Hz, 1H), 8.41 (s, 1H), 7.10 (d, J=7.9 Hz, 1H), 4.66-4.57 (m, 1H), 4.37-4.27 (m, 1H), 4.22 (ddq, J=16.2, 8.5, 4.3 Hz, 1H), 3.09-2.96 (m, 2H), 2.83 (dd, J=12.0, 10.6 Hz, 1H), 2.77 (dd, J=12.6, 11.0 Hz, 1H), 2.58 (s, 4H), 2.47 (s, 4H), 2.31 (s, 3H), 2.24-2.15 (m, 1H), 2.10 (ddt, J=10.8, 7.7, 4.0 Hz, 1H), 1.19 (q, J=11.8 Hz, 1H), 1.04 (d, J=6.5 Hz, 3H).

The following compounds were synthesized in an analogous manner.

### Example 189: N-[(3R,5S)-1-(5-Cyano-pyrido[3,4-b]pyrazin-8-yl)-5-methyl-piperidin-3-yl]-3-(1-methyl-piperidin-4-yl)-propionamide

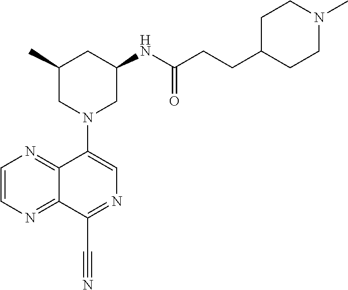 [SMILES: C[C@H]1C[C@@H](NC(=O)CCC2CCN(C)CC2)CN(c2cnc(C#N)c3nccnc23)C1]

The title compound was prepared from -((3R,5S)-3-Amino-5-methyl-piperidin-1-yl)-pyrido[3,4-b]pyrazine-5-carbonitrile hydrochloride and 3-(1-methyl-4-piperidinyl)propanoic acid hydrochloride. MS: 422 [M+H]⁺. ¹H NMR (400 MHz, Chloroform-d) δ 9.02 (d, J=1.7 Hz, 1H), 8.96 (d, J=1.7 Hz, 1H), 8.43 (s, 1H), 5.36 (d, J=7.5 Hz, 1H), 4.59 (ddt, J=12.2, 4.1, 1.8 Hz, 1H), 4.31 (ddt, J=12.7, 3.8, 1.5 Hz, 1H), 4.21 (tdd, J=10.5, 7.9, 4.5 Hz, 1H), 2.87-2.72 (m, 4H), 2.26 (s, 3H), 2.24-2.14 (m, 3H), 2.08 (ddd, J=10.9, 7.0, 4.0 Hz, 1H), 1.89 (t, J=10.9 Hz, 2H), 1.67 (d, J=10.6 Hz, 2H), 1.63-1.57 (m, 2H), 1.35-1.24 (m, 3H), 1.15 (q, J=11.7 Hz, 1H), 1.02 (d, J=6.6 Hz, 3H).

### Example 190: N-[(3R,5S)-1-(5-Cyano-pyrido[3,4-b]pyrazin-8-yl)-5-methyl-piperidin-3-yl]-2-(4-methyl-piperazin-1-yl)-propionamide

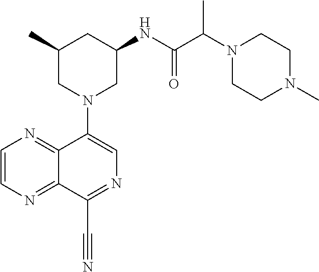 [SMILES: CC(C(=O)N[C@@H]1C[C@H](C)CN(c2cnc(C#N)c3nccnc23)C1)N1CCN(C)CC1]

The title compound was prepared from -((3R,5S)-3-Amino-5-methyl-piperidin-1-yl)-pyrido[3,4-b]pyrazine-5-carbonitrile hydrochloride and 2-(4-methylpiperazin-1-yl)propanoic acid dihydrochloride. MS:423 [M+H]⁺. ¹H NMR (400 MHz, Chloroform-d) δ 9.02 (t, J=1.7 Hz, 1H), 8.97 (d, J=1.6 Hz, 1H), 8.42 (s, 1H), 4.66-4.59 (m, 1H), 4.58-4.52 (m, 1H), 4.36-4.28 (m, 1H), 4.22-4.13 (m, 1H), 3.13-3.03 (m, 1H), 2.84-2.73 (m, 2H), 2.64-2.42 (m, 6H), 2.32 (s, 3H), 2.14 (s, 3H), 1.25 (d, J=7.3 Hz, 3H), 1.21-1.11 (m, 1H), 1.04 (d, J=6.5 Hz, 3H).

### Example 191: N-[(3R,5S)-1-(8-Cyano-quinazolin-5-yl)-5-methyl-piperidin-3-yl]-3-(1-methyl-piperidin-4-yl)-propionamide

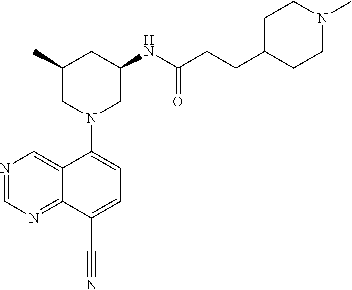 [SMILES: C[C@H]1C[C@@H](NC(=O)CCC2CCN(C)CC2)CN(c2ccc(C#N)c3ncncc23)C1]

The title compound was prepared from 5-((3R,5S)-3-Amino-5-methyl-piperidin-1-yl)-quinazoline-8-carbonitrile hydrochloride and 3-(1-methyl-4-piperidinyl)propanoic acid hydrochloride. MS: 421 [M+H]. ¹H NMR (400 MHz, Chloroform-d) δ 9.61 (s, 1H), 9.40 (s, 1H), 8.11 (d, J=8.2 Hz, 1H), 7.13 (d, J=8.2 Hz, 1H), 5.33 (d, J=7.3 Hz, 1H), 4.24 (dtd, J=15.6, 7.6, 3.9 Hz, 1H), 3.98 (ddt, J=11.7, 4.1, 1.9 Hz, 1H), 3.62 (ddt, J=12.4, 3.9, 1.8 Hz, 1H), 2.84 (d, J=11.8 Hz, 2H), 2.73-2.58 (m, 2H), 2.26 (s, 3H), 2.23-2.03 (m, 4H), 1.89 (t, J=10.8 Hz, 2H), 1.72-1.64 (m, 2H), 1.64-1.55 (m, 2H), 1.36-1.21 (m, 3H), 1.11 (q, J=11.9 Hz, 1H), 1.0 (d, J=6.5 Hz, 3H).

### Example 192: N-[(3R,5S)-1-(8-Cyano-quinoxalin-5-yl)-5-methyl-piperidin-3-yl]-2-(4-fluoro-1-methyl-piperidin-4-yl)-acetamide

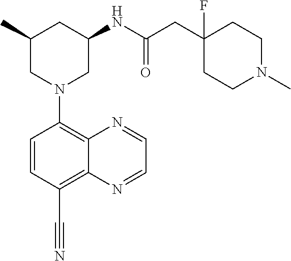

The title compound was prepared from 8-((3R,5S)-3-Amino-5-methyl-piperidin-1-yl)-quinoxaline-5-carbonitrile hydrochloride and (4-Fluoro-1-methyl-piperidin-4-yl)-acetic acid. MS:425 [M+H]⁺. ¹H NMR (400 MHz, Chloroform-d) δ 8.94 (d, J=1.8 Hz, 1H), 8.82 (d, J=1.8 Hz, 1H), 8.0 (d, J=8.4 Hz, 1H), 7.20 (d, J=8.4 Hz, 1H), 5.87 (t, J=6.2 Hz, 1H), 4.40-4.21 (m, 3H), 2.82 (dd, J=12.1, 10.2 Hz, 1H), 2.75 (dd, J=12.6, 10.8 Hz, 1H), 2.70-2.59 (m, 2H), 2.54 (d, J=23.8 Hz, 2H), 2.36-2.28 (m, 5H), 2.15 (dt, J=13.3, 2.4 Hz, 1H), 2.08-1.98 (m, 1H), 1.96-1.89 (m, 2H), 1.84-1.74 (m, 1H), 1.27-1.11 (m, 2H), 0.99 (d, J=6.6 Hz, 3H).

### Example 193: N-[(3R,5S)-1-(8-Cyano-quinoxalin-5-yl)-5-methyl-piperidin-3-yl]-3-imidazol-1-yl-propionamide

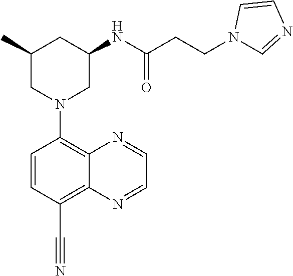 [SMILES: C[C@H]1C[C@@H](NC(=O)CCn2ccnc2)CN(c2ccc(C#N)c3nccnc23)C1]

The title compound was prepared from 8-((3R,5S)-3-Amino-5-methyl-piperidin-1-yl)-quinoxaline-5-carbonitrile hydrochloride and 3-(1H-imidazol-1-yl)propanoic acid. MS: 390 [M+H]⁺. ¹H NMR (400 MHz, Chloroform-d) δ 8.95 (d, J=1.7 Hz, 1H), 8.81 (d, J=1.7 Hz, 1H), 8.01 (d, J=8.3 Hz, 1H), 7.49 (t, J=1.1 Hz, 1H), 7.15 (d, J=8.4 Hz, 1H), 7.04 (t, J=1.1 Hz, 1H), 6.94 (t, J=1.3 Hz, 1H), 5.61 (d, J=7.2 Hz, 1H), 4.34 (t, J=6.3 Hz, 2H), 4.30-4.14 (m, 3H), 2.78-2.64 (m, 2H), 2.60 (dd, J=6.8, 5.8 Hz, 2H), 2.12-1.98 (m, 2H), 1.10 (q, J=11.4 Hz, 1H), 0.97 (d, J=6.5 Hz, 3H).

### Example 194: N-[(3R,5S)-1-(8-Cyano-quinoxalin-5-yl)-5-methyl-piperidin-3-yl]-3-morpholin-4-yl-propionamide

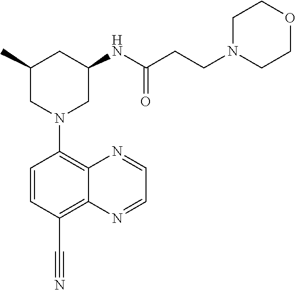 [SMILES: C[C@H]1C[C@@H](NC(=O)CCN2CCOCC2)CN(c2ccc(C#N)c3nccnc23)C1]

The title compound was prepared from 8-((3R,5S)-3-Amino-5-methyl-piperidin-1-yl)-quinoxaline-5-carbonitrile hydrochloride and 3-morpholin-4-ylpropanoic acid. MS: 409 [M+H]⁺. ¹H NMR (400 MHz, Chloroform-d) δ 8.94 (d, J=1.8 Hz, 1H), 8.83 (d, J=1.7 Hz, 1H), 8.28 (s, 1H), 7.99 (d, J=8.4 Hz, 1H), 7.21 (d, J=8.4 Hz, 1H), 4.42 (ddd, J=12.1, 4.2, 2.1 Hz, 1H), 4.39-4.30 (m, 1H), 4.20 (dtd, J=14.9, 7.2, 4.4 Hz, 1H), 3.74 (t, J=4.7 Hz, 4H), 2.82-2.69 (m, 2H), 2.69-2.63 (m, 2H), 2.54 (s, 4H), 2.42 (t, J=6.1 Hz, 2H), 2.21-2.11 (m, 1H), 2.02 (tdd, J=13.7, 8.6, 5.3 Hz, 1H), 1.10 (q, J=11.9 Hz, 1H), 0.99 (d, J=6.6 Hz, 3H).

### Example 195: N-[(3R,5S)-1-(8-Cyano-quinoxalin-5-yl)-5-methyl-piperidin-3-yl]-3-dimethylsulfamoyl-propionamide

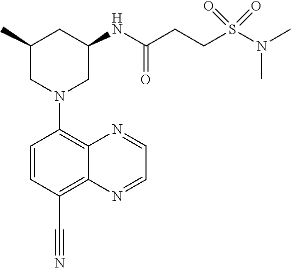

The title compound was prepared from 8-((3R,5S)-3-Amino-5-methyl-piperidin-1-yl)-quinoxaline-5-carbonitrile hydrochloride and 3-(dimethylsulfamoyl)propanoic acid. MS: 431 [M+H]⁺. ¹H NMR (400 MHz, Chloroform-d) δ 8.96 (d, J=1.8 Hz, 1H), 8.84 (d, J=1.8 Hz, 1H), 8.0 (d, J=8.3 Hz, 1H), 7.17 (d, J=8.4 Hz, 1H), 5.87 (d, J=7.1 Hz, 1H), 4.32-4.16 (m, 3H), 3.28 (td, J=7.1, 3.2 Hz, 2H), 2.89 (s, 6H), 2.80-2.67 (m, 3H), 2.19-2.11 (m, 1H), 2.05 (dtd, J=10.5, 6.7, 3.6 Hz, 1H), 1.32-1.16 (m, 2H), 1.01 (d, J=6.6 Hz, 3H).

### Example 196: (S)—N-[(3R,5S)-1-(5-Cyano-pyrido[3,4-b]pyrazin-8-yl)-5-methyl-piperidin-3-yl]-2-hydroxy-3-methyl-butyramide

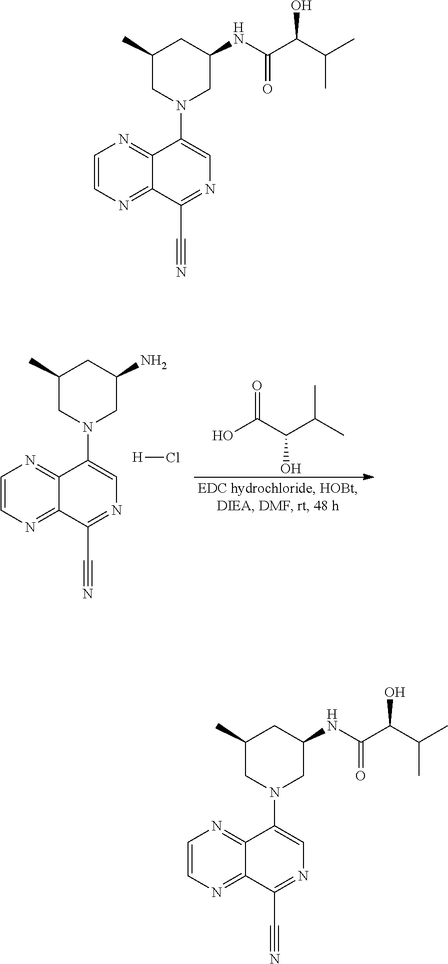

In a 20 mL scintillation vial, under nitrogen, 8-((3R,5S)-3-Amino-5-methyl-piperidin-1-yl)-pyrido[3,4-b]pyrazine-5-carbonitrile hydrochloride (80.0 mg; 0.262 mmol) (s)-(+)-2-hydroxy-3-methylbutyric acid (34.1 mg; 0.289 mmol), 1-(3-dimethylaminopropyl)-3-ethylcarbodiimide hydrochloride (60.4 mg; 0.315 mmol), 1-hydroxybenzotriazole hydrate (48.2 mg; 0.315 mmol) and DIPEA (228.6 μl; 1.312 mmol) were dissolved in anhydrous DMF (5.0 ml). The orange solution was stirred at room temperature for 2 days. The orange solution was concentrated under reduced pressure. The residue was dissolved in DCM, absorbed on a PuriFlash 4 g 30u column and purified by chromatography on a PuriFlash 12 g 30u column (Hexanes-AcOEt 30% for 5 column volumes, Hexanes-AcOEt 30-100% for 10 column volumes, AcOEt for 5 column volumes). The pure fractions were concentrated under reduced pressure and the residue was dissolved in acetonitrile and lyophilized to give the title compound as a orange solid (69.0 mg; 66.9%). MS:369 [M+H]⁺. ¹H NMR (400 MHz, Chloroform-d) δ 9.03 (d, J=1.7 Hz, 1H), 8.96 (d, J=1.7 Hz, 1H), 8.43 (s, 1H), 6.50 (d, J=7.8 Hz, 1H), 4.56 (ddt, J=12.1, 4.1, 1.8 Hz, 1H), 4.31 (ddt, J=12.6, 3.7, 1.6 Hz, 1H), 4.24 (dddd, J=14.7, 10.5, 8.0, 4.2 Hz, 1H), 4.01 (d, J=3.1 Hz, 1H), 2.90 (dd, J=12.1, 10.4 Hz, 1H), 2.79 (dd, J=12.7, 10.9 Hz, 1H), 2.25-2.05 (m, 4H), 1.24 (q, J=11.7 Hz, 1H), 1.04 (d, J=7.0 Hz, 3H), 1.03 (d, J=6.7 Hz, 3H), 0.87 (d, J=6.9 Hz, 3H).

### Example 197 (Isomer 1): 5-((R)-5-Amino-3,3-difluoro-piperidin-1-yl)-quinoline-8-carbonitrile & Example 198 (Isomer 2): 5-((R)-5-Amino-3,3-difluoro-piperidin-1-yl)-quinoline-8-carbonitrile

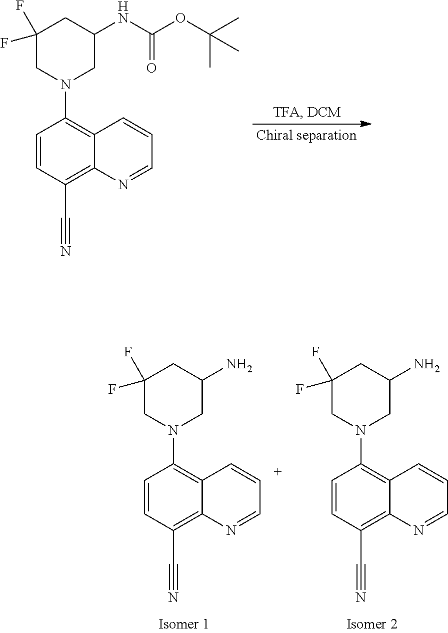 [SMILES: CC(C)(C)OC(=O)NC1CN(c2ccc(C#N)c3ncccc23)CC(F)(F)C1 ; N#Cc1ccc(N2CC(N)CC(F)(F)C2)c2cccnc12 ; N#Cc1ccc(N2CC(N)CC(F)(F)C2)c2cccnc12]

In a 100 mL round bottom flask, [1-(8-Cyano-quinolin-5-yl)-5,5-difluoro-piperidin-3-yl]-carbamic acid tert-butyl ester (720.0 mg; 1.854 mmol) was dissolved in anhydrous DCM (10.0 ml). TFA (4.3 ml; 55.611 mmol) was added to the yellow solution and the orange solution was stirred at room temperature for 1 h. The reaction mixture was concentrated under reduced pressure. The residue was dissolved in methanol and filter through 2 5 g SiliCycle SilicaPrep Carbonate column and the resulting solution was concentrated under reduced pressure and dried under vacuo to give 922 mg of a yellow solid. The 2 isomers were obtained by separation on chiral prep-HPLC under the following conditions: column, ADH, Prep SFC-P100; mobile phase, methanol+20 Mm NH₄OH, 40° C./80 bar, 100 g/min; detector, PDA.

Isomer 1: a white solid (137.0 mg; 25.6%). ¹H NMR (400 MHz, DMSO-d₆) δ 9.09 (dd, J=4.2, 1.6 Hz, 1H), 8.60 (dd, J=8.6, 1.7 Hz, 1H), 8.39 (s, 2H), 8.32 (d, J=8.0 Hz, 1H), 7.74 (dd, J=8.6, 4.2 Hz, 1H), 7.39 (d, J=8.0 Hz, 1H), 3.87 (s, 1H), 3.67 (dd, J=12.3, 3.6 Hz, 1H), 3.64-3.45 (m, 2H), 3.14 (dd, J=12.5, 8.9 Hz, 1H), 2.65 (dq, J=18.4, 7.5, 5.7 Hz, 1H), 2.29 (ddt, J=23.1, 13.7, 8.8 Hz, 1H). MS:289 [M+H]⁺. Rt 2.70 min. ee 96.0%.

Isomer 2: a white solid (136.0 mg; 25.4%). MS:289 [M+H]⁺. ¹H NMR (400 MHz, Chloroform-d) δ 9.09 (dd, J=4.2, 1.7 Hz, 1H), 8.65 (dd, J=8.6, 1.7 Hz, 1H), 8.04 (d, J=7.9 Hz, 1H), 7.55 (dd, J=8.6, 4.2 Hz, 1H), 7.12 (d, J=7.9 Hz, 1H), 3.50 (ddd, J=12.7, 8.4, 4.1 Hz, 2H), 3.43 (dd, J=11.7, 3.5 Hz, 1H), 3.29-3.12 (m, 1H), 2.93 (t, J=10.0 Hz, 1H), 2.58-2.42 (m, 1H), 1.99-1.81 (m, 1H), 1.45 (s, 2H). Rt 3.11 min. ee 96%.

### Example 199: (3R,5S)-1-(5-Methoxy-pyrido[3,4-b]pyrazin-8-yl)-5-methyl-piperidin-3-ylamine

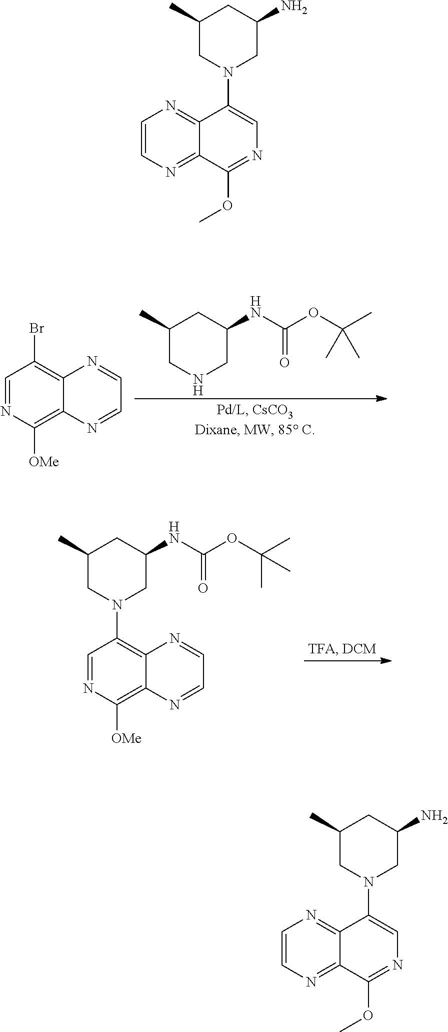 [SMILES: COc1ncc(N2C[C@@H](C)C[C@@H](N)C2)c2nccnc12 ; COc1ncc(Br)c2nccnc12 ; C[C@@H]1CNC[C@H](NC(=O)OC(C)(C)C)C1 ; COc1ncc(N2C[C@@H](C)C[C@@H](NC(=O)OC(C)(C)C)C2)c2nccnc12 ; COc1ncc(N2C[C@@H](C)C[C@@H](N)C2)c2nccnc12]

[(3R,5S)-1-(5-Methoxy-pyrido[3,4-b]pyrazin-8-yl)-5-methyl-piperidin-3-yl]-carbamic acid tert-butyl ester: In a 30 mL microwave vial, 8-Bromo-5-methoxy-pyrido[3,4-b]pyrazine (320.0 mg; 1.333 mmol), ((3R,5S)-5-Methyl-piperidin-3-yl)-carbamic acid tert-butyl ester (428.5 mg; 2.00 mmol), chloro(2-dicyclohexylphosphino-2′,6′-di-i-propoxy-1,1′-biphenyl)[2-(2-aminoethylphenyl)]palladium(ii), methyl-t-butylether adduct (54.4 mg; 0.067 mmol), 2-dicyclohexylphosphino-2′,6′-diisopropoxy-1,1′-biphenyl (31.1 mg; 0.067 mmol) and cesium carbonate (1.3 g; 3.999 mmol) were suspended in anhydrous dioxane (12.0 ml). The tube was sealed and flushed with nitrogen for 15 min and the colorless cloudy solution was microwaved at 120° C. for 4 hours. The reaction mixture was concentrated under reduced pressure, the residue was suspended in DCM, filtered on celite and concentrated under reduced pressure. The residue was dissolved in DCM, adsorb on a PuriFlash 4 g 30u column and purified by chromatography on a PuriFlash 40 g 30u column (Hexane-AcOEt 20% for 5 column volumes, Hexane-AcOEt 20-100% for 15 column volumes), major product eluted with AcOEt 59-68% (lambda=240). The pure fractions were concentrated under reduced pressure and the yellow solid was dried under vacuo to give the title compound as a yellow solid (200.0 mg; 40.2%). MS:374 [M+H]⁺.

(3R,5S)-1-(5-Methoxy-pyrido[3,4-b]pyrazin-8-yl)-5-methyl-piperidin-3-ylamine: In a 20 mL scintillation vial, [(3R,5S)-1-(5-Methoxy-pyrido[3,4-b]pyrazin-8-yl)-5-methyl-piperidin-3-yl]-carbamic acid tert-butyl ester (190.0 mg; 0.509 mmol) was dissolved in anhydrous DCM (2.0 ml). TFA (1.9 ml; 25.438 mmol) was added to the orange solution and the tan solution was stirred at room temperature for 1 h. The reaction mixture was concentrated under reduced pressure, diluted with methanol (10 mL) and filtered over a SilicCycle Si-Carbonate 5 g. The tan solution was concentrated under reduced pressure. The residue was dissolved in DCM, adsorb on a PuriFlash 6 g 50u NH₂ column and purified by chromatography on a PuriFlash 20 g 30u NH₂ column (DCM for 10 column volumes), major product eluted after 1.1 to 2.6 column volumes (lambda max=232). The pure fractions were concentrated under reduced to give the title compound as an orange oil (121.0 mg; 85.9%). MS: 274 [M+H]⁺. ¹H NMR (400 MHz, Chloroform-d) δ 9.01 (d, J=1.9 Hz, 1H), 8.89 (d, J=1.8 Hz, 1H), 7.87 (s, 1H), 4.19 (s, 3H), 3.82 (ddt, J=10.7, 3.9, 1.7 Hz, 1H), 3.63 (ddt, J=11.1, 3.7, 1.8 Hz, 1H), 3.25 (ddt, J=11.3, 10.2, 4.1 Hz, 1H), 2.36 (t, J=10.8 Hz, 1H), 2.30 (t, J=10.8 Hz, 1H), 2.17-2.05 (m, 2H), 0.99 (d, J=6.4 Hz, 3H), 0.90 (td, J=12.7, 11.1 Hz, 1H).

### Examples 200: N-(1-methylpiperidin-4-yl){[(3R,5S)-5-methyl-1-[8-(trifluoromethyl)quinolin-5-yl]piperidin-3-yl]amino}sulfonamide

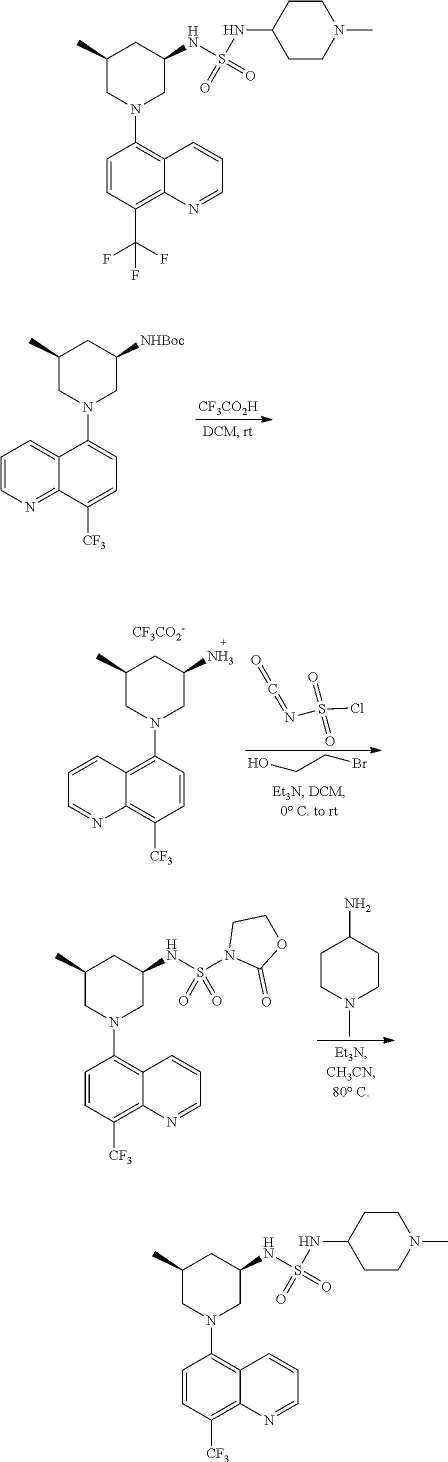

(3R,5S)-5-Methyl-1-[8-(trifluoromethyl)quinolin-5-yl]piperidin-3-aminium trifluoroacetate: To a stirred suspension of N-[(3R,5S)-5-methyl-1-[8-(trifluoromethyl)quinolin-5-yl]piperidin-3-yl]carbamate (1.11 g, 2.71, 1.0 eq.) in dichloromethane (4.0 ml; 3.60 V) at room temperature was added TFA (1.0 ml; 13.07 mmol; 4.82 eq.). The resulting mixture was stirred at room temperature for 3.5 h. The solvent was removed. The residue was evaporated with toluene (10 mL) twice to give a pale yellow viscous oil, which was used directly for the next step without purification. MS: 310 [M+H]⁺.

N-[(3R,5S)-5-methyl-1-[8-(trifluoromethyl)quinolin-5-yl]piperidin-3-yl]-2-oxo-1,3-oxazolidine-3-sulfonamide: To a stirred solution of chlorosulfonyl isocyanate (0.29 ml; 3.31 mmol; 2.0 eq.) in DCM (5 mL) at ° C. was added 2-bromoethanol (0.23 ml; 3.31 mmol; 2.0 eq.). The resulting mixture was warmed to room temperature for 30 min, and added to a stirred solution (3R,5S)-5-methyl-1-[8-(trifluoromethyl)quinolin-5-yl]piperidin-3-aminium trifluoroacetate (700.0 mg; 1.65 mmol; 1.0 eq.) in DCM (5 mL) and TEA (368.09 mg; 3.64 mmol; 2.20 eq.) at 0° C. The resulting mixture was warmed to room temperature and stirred for 1.5 h. The reaction was quenched by adding water (10 mL), extracted with EtOAc (20 mL×2). The organic layer was drived over Na₂SO₄, and concentrated. The residue was used directly for next step without further purification. MS: 459 [M+H]⁺.

N-[(3R,5S)-5-methyl-1-[8-(trifluoromethyl)quinolin-5-yl]piperidin-3-yl][(1-methylpiperidin-4-yl)amino]sulfonamide: To a stirred solution of N-[(3R,5S)-5-methyl-1-[8-(trifluoromethyl)quinolin-5-yl]piperidin-3-yl]-2-oxo-1,3-oxazolidine-3-sulfonamide (97.65 mg; 0.21 mmol; 1.0 eq.) in acetonitrile (2 mL, 20 V) was added TEA (107.77 mg; 1.07 mmol; 5.0 eq.), followed by 4-amino-1-methylpiperidine (48.64 mg; 0.43 mmol; 2.0 eq.). The resulting mixture was stirred at 80° C. for 1.5 h. The mixture was diluted with EtOAc (30 mL), washed with water (10 mL) and brine (10 mL) dried over Na₂SO₄, and concentrated. The crude was purified by prep-HPLC (ACN/water with 0.10% NH₄OH as modifier) to yield the title compound as white solid (78.8 mg; 75%). MS: 486 [M+H]⁺. ¹H NMR (400 MHz, CDCl₃) δ 9.03 (dd, J=4.2, 1.7 Hz, 1H), 8.43 (dd, J=8.6, 1.8 Hz, 1H), 7.96 (d, J=8.0 Hz, 1H), 7.48 (dd, J=8.6, 4.2 Hz, 1H), 7.05 (d, J=8.0 Hz, 1H), 4.72 (br s, 1H), 4.45 (s, 1H), 3.79-3.64 (m, 2H), 3.32-3.24 (m, 2H), 2.96-2.90 (m, 2H), 2.52 (t, J=11.1 Hz, 1H), 2.36 (s, 3H), 2.35 (t, J=11.1 Hz, 1H) 2.29-2.18 (m, 3H), 2.16-1.94 (m, 2H), 1.80-1.65 (m, 3H), 1.06 (d, J=12.0 Hz, 1H), 0.99 (d, J=6.6 Hz, 3H).

The following compounds were synthesized in an analogous manner.

### Examples 201: N-[2-(1-methylpiperidin-4-yl)ethyl]{[(3R,5S)-5-methyl-1-[8-(trifluoromethyl) quinolin-5-yl]piperidin-3-yl]amino}sulfonamide

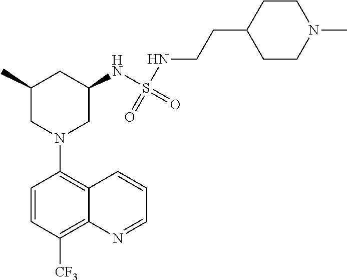

The title compound was prepared from N-[(3R,5S)-5-methyl-1-[8-(trifluoromethyl)quinolin-5-yl]piperidin-3-yl]-2-oxo-1,3-oxazolidine-3-sulfonamide and 2-(1-methylpiperidin-4-yl)ethan-1-amine. MS: 514 [M+H]⁺. ¹H NMR (400 MHz, CDCl₃) δ 9.07-9.01 (m, 1H), 8.44 (dd, J=8.5, 1.5 Hz, 1H), 7.97 (d, J=8.0 Hz, 1H), 7.50 (dd, J=8.6, 4.2 Hz, 1H), 7.07 (d, J=8.0 Hz, 1H), 4.23 (d, J=31.9 Hz, 2H), 3.77-3.69 (m, 2H), 3.32 (d, J=11.4 Hz, 1H), 3.18-2.97 (m, 3H), 2.65-2.53 (m, 1H), 2.46-2.25 (m, 3H), 2.27 (d, J=12.5 Hz, 1H), 2.22-2.08 (m, 3H), 1.74-1.68 (m, 2H), 1.58-1.50 (m, 6H), 1.05 (q, J=11.6 Hz, 1H), 1.02 (d, J=6.6 Hz, 3H).

### Examples 202: N-{[(3R,5S)-5-methyl-1-[8-(trifluoromethyl)quinolin-5-yl]piperidin-3-yl]sulfamoyl}-2-(1-methylpiperidin-4-yl)acetamide

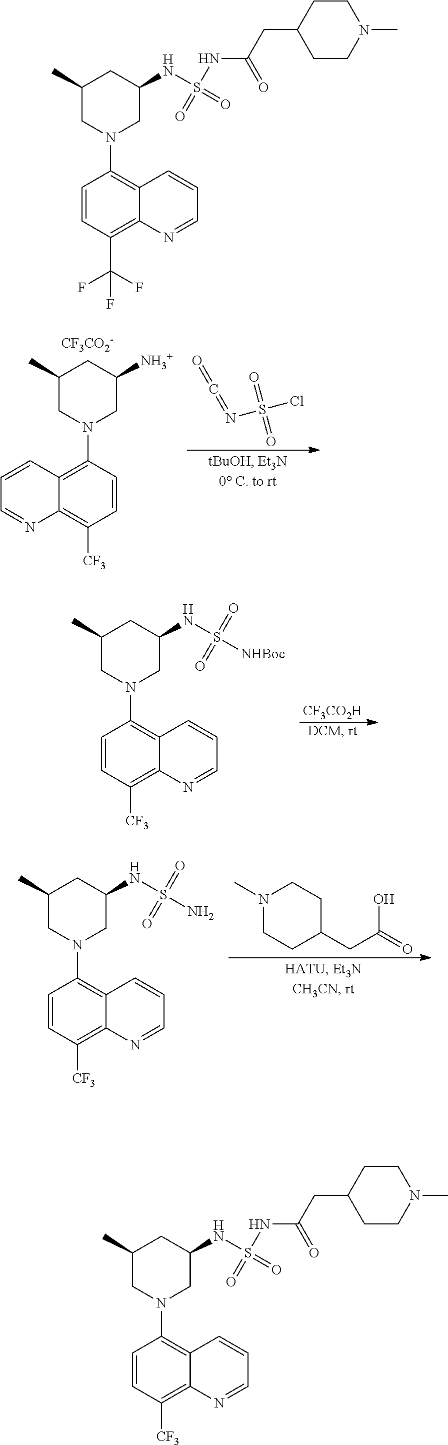

tert-butyl N-{[(3R,5S)-5-methyl-1-[8-(trifluoromethyl)quinolin-5-yl]piperidin-3-yl]sulfamoyl}carbamate: To a stirred solution of tBuOH (20.24 mg; 0.27 mmol; 1.40 eq.) in DCM (2 mL) at room temperature was added chlorosulfonyl isocyanate (0.02 ml; 0.20 mmol; 1.0 eq.). The resulting mixture was stirred at room temperature for 15 min, and was added to a solution of (3R,5S)-5-methyl-1-[8-(trifluoromethyl)quinolin-5-yl]piperidin-3-aminium trifluoroacetate (82.55 mg; 0.20 mmol; 1.0 eq.) in DCM (2 mL) at 0° C., followed by adding TEA (21.71 mg; 0.21 mmol; 1.10 eq.). The resulting mixture was stirred at room temperature after addition for 1 h. The reaction was quenched by adding water (10 mL), extracted with EtOAc (10 mL×2). The organic layer was drived over Na₂SO₄, and concentrated to give a pale yellow viscous oil. The residue was used directly for next step. MS:489 [M+H]⁺.

N-[(3R,5S)-5-methyl-1-[8-(trifluoromethyl)quinolin-5-yl]piperidin-3-yl]aminosulfonamide: To a stirred suspension of tert-butyl N-{[(3R,5S)-5-methyl-1-[8-(trifluoromethyl)quinolin-5-yl]piperidin-3-yl]sulfamoyl}carbamate (410.36 mg; 0.84 mmol; 1.0 eq.) in DCM (2 mL) at room temperature was added TFA (3 mL, 39.21 mmol, 46.7 eq.). The resulting mixture was stirred at room temperature for 3 h. The solvent was removed to give a viscous oil. The residue was used directly for next step without further purification. MS:389 [M+H]⁺.

N-{[(3R,5S)-5-methyl-1-[8-(trifluoromethyl)quinolin-5-yl]piperidin-3-yl]sulfamoyl}-2-(1-methylpiperidin-4-yl)acetamide: To a flask containing N-[(3R,5S)-5-methyl-1-[8-(trifluoromethyl)quinolin-5-yl]piperidin-3-yl]aminosulfonamide (77.68 mg; 0.20 mmol; 1.0 eq.) at room temperature was added MeCN (2 mL), 2-(1-methylpiperidin-4-yl)acetic acid (47.16 mg; 0.30 mmol; 1.50 eq.) and TEA (121.43 mg; 1.20 mmol; 6.0 eq.), followed by HATU (114.07 mg; 0.30 mmol; 1.50 eq.). The resulting mixture was stirred at room temperature for 18 h. The mixture was diluted with EtOAc (20 mL), washed with water (10 mL) and brine (10 mL) dried over Na₂SO₄, and concentrated. The crude was purified by prep-HPLC (ACN/water with 0.10% NH₄OH as modifier) to yield the title compound as white solid (31.4 mg; 30%). MS:528 [M+H]⁺. ¹H NMR (400 MHz, CDCl₃) δ 9.02 (dd, J=4.2, 1.7 Hz, 1H), 8.59 (s, 1H), 8.44 (dd, J=8.6, 1.8 Hz, 1H), 7.95 (d, J=8.0 Hz, 1H), 7.48 (dd, J=8.6, 4.2 Hz, 1H), 7.04 (d, J=8.0 Hz, 1H), 3.79-3.71 (m, 2H), 3.37-3.26 (m, 2H), 2.60 (s, 3H), 2.56 (t, J=12.0 Hz, 1H), 2.44 (q, J=10.4, 9.7 Hz, 2H), 2.34 (t, J=11.4 Hz, 1H), 2.23-2.15 (m, 4H), 2.11-2.04 (m, 1H), 1.99-1.90 (d, J=3.6 Hz, 1H), 1.86-1.79 (m, 2H), 1.69-1.55 (m, 2H), 1.04 (q, J=11.8 Hz, 1H), 0.95 (d, J=6.6 Hz, 3H).

The following compounds were synthesized in an analogous manner.

### Examples 203: N-{[(3R,5S)-5-methyl-1-[8-(trifluoromethyl)quinolin-5-yl]piperidin-3-yl]sulfa moyl}-2-(morpholin-4-yl)acetamide

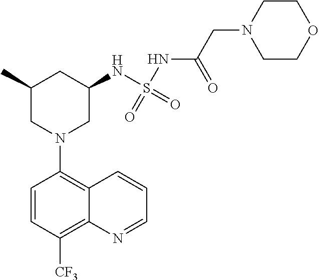 [SMILES: C[C@H]1C[C@@H](NS(=O)(=O)NC(=O)CN2CCOCC2)CN(c2ccc(C(F)(F)F)c3ncccc23)C1]

The title compound was prepared from N-[(3R,5S)-5-methyl-1-[8-(trifluoromethyl)quinolin-5-yl]piperidin-3-yl]aminosulfonamide and 2-morpholinoacetic acid hydrochloride. MS:516 [M+H]⁺. ¹H NMR (400 MHz, CDCl₃) δ 9.05 (dd, J=4.2, 1.8 Hz, 1H), 8.42 (dd, J=8.6, 1.8 Hz, 1H), 7.97 (d, J=8.0 Hz, 1H), 7.49 (dd, J=8.6, 4.2 Hz, 1H), 7.06 (d, J=8.0 Hz, 1H), 5.22 (d, J=6.7 Hz, 1H), 3.83-3.71 (m, 1H), 3.71-3.66 (m, 5H), 3.34-3.29 (m, 1H), 3.15-2.99 (m, 2H), 2.63 (t, J=10.9 Hz, 1H), 2.54-2.51 (m, 4H), 2.37 (t, J=11.3 Hz, 1H), 2.25-2.15 (m, 1H), 2.15-2.08 (m, 1H), 1.11 (q, J=11.9 Hz, 1H), 1.0 (d, J=6.5 Hz, 3H).

### Examples 204: (3R)—N-[(3R,5S)-5-methyl-1-[8-(trifluoromethyl)quinolin-5-yl]piperidin-3-yl]pyrrolidine-3-carboxamide

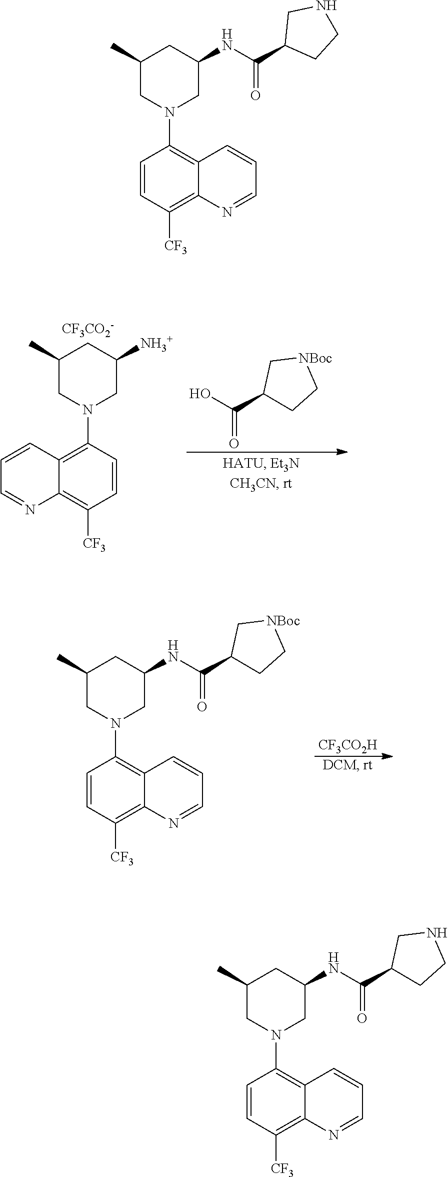

tert-butyl (3R)-3-{[(3R,5S)-5-methyl-1-[8-(trifluoromethyl)quinolin-5-yl]piperidin-3-yl]carbamoyl}pyrrolidine-1-carboxylate: To a flask containing (3R,5S)-5-methyl-1-[8-(trifluoromethyl)quinolin-5-yl]piperidin-3-aminium trifluoroacetate (75.76 mg; 0.17 mmol; 1.0 eq.) at room temperature was added MeCN (2.0 ml), (R)-1-boc-pyrrolidine-3-carboxylic acid (54.89 mg; 0.26 mmol; 1.50 eq.) and TEA (103.21 mg; 1.02 mmol; 6.0 eq.), followed by HATU (96.96 mg; 0.26 mmol; 1.50 eq.). The resulting mixture was stirred at room temperature for 1 h. The mixture was diluted with EtOAc (20 mL), washed with water (10 mL) and brine (10 mL). The organic layer was dried over Na₂SO₄, and concentrated to give a pale yellow viscous oil, which was used directly for next step. MS:507 [M+H]⁺.

(3R)—N-[(3R,5S)-5-methyl-1-[8-(trifluoromethyl)quinolin-5-yl]piperidin-3-yl]pyrrolidine-3-carboxamide: To a stirred solution of tert-butyl (3R)-3-{[(3R,5S)-5-methyl-1-[8-(trifluoromethyl)quinolin-5-yl]piperidin-3-yl]carbamoyl}pyrrolidine-1-carboxylate (81.05 mg; 0.16 mmol; 1.0 eq.) in dichloromethane (4.0 mL) at room temperature was added TFA (1.0 ml; 13.07 mmol). The resulting mixture was stirred at room temperature for 2 h. The solvent was removed. The crude was purified by prep-HPLC (ACN/water with 0.10% NH₄OH as modifier) to yield the title compound as white solid (54.1 mg; 84%). MS:407 [M+H]⁺. ¹H NMR (400 MHz, CDCl₃) δ 9.03 (dd, J=4.4, 1.7 Hz, 1H), 8.49 (dd, J=8.6, 1.8 Hz, 1H), 7.95 (d, J=8.0 Hz, 1H), 7.50 (dd, J=8.5, 4.1 Hz, 1H), 7.04 (d, J=8.0 Hz, 1H), 6.14 (d, J=7.7 Hz, 1H), 4.31-4.21 (m, 1H), 3.64 (d, J=11.1 Hz, 1H), 3.48-3.30 (m, 4H), 3.08-3.02 (m, 1H), 2.46-2.39 (m, 2H), 2.35-2.27 (m, 1H), 2.22-2.11 (m, 3H), 1.73-1.47 (m, 3H), 1.09 (q, J=12.0 Hz, 1H), 1.01 (d, J=6.3 Hz, 3H).

The following compounds were synthesized in an analogous manner.

### Examples 205: 3-fluoro-N-[(3R,5S)-5-methyl-1-[8-(trifluoromethyl)quinolin-5-yl]piperidin-3-yl]pyrrolidine-3-carboxamide

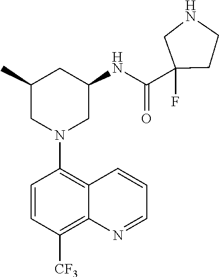 [SMILES: C[C@H]1C[C@@H](NC(=O)C2(F)CCNC2)CN(c2ccc(C(F)(F)F)c3ncccc23)C1]

The title compound was prepared from (3R,5S)-5-methyl-1-[8-(trifluoromethyl)quinolin-5-yl]piperidin-3-aminium trifluoro acetate and 1-[(tert-butoxy)carbonyl]-3-fluoropyrrolidine-3-carboxylic acid. MS: 425 [M+H]⁺. ¹H NMR (400 MHz, CDCl₃) δ 9.04 (dd, J=4.2, 1.8 Hz, 1H), 8.53 (dt, J=8.6, 1.9 Hz, 1H), 7.96 (d, J=8.0 Hz, 1H), 7.52 (ddd, J=8.6, 4.2, 1.1 Hz, 1H), 7.07 (d, J=8.0 Hz, 1H), 6.36 (t, J=7.0 Hz, 1H), 4.38-4.30 (m, 1H), 3.73-3.67 (m, 1H), 3.37-3.33 (m, 1H), 3.26 (td, J=12.3, 11.3, 3.8 Hz, 1H), 3.21-3.07 (m, 3H), 2.50-2.41 (m, 2H), 2.29-2.26 (m, 1H), 2.26-2.0 (m, 3H), 1.10 (q, J=11.9 Hz, 1H), 1.03 (d, J=6.5 Hz, 3H).

### Examples 206: N-[(3R,5S)-1-(8-cyanoquinolin-5-yl)-5-(trifluoromethyl)piperidin-3-yl]-3-fluoropyrrolidine-3-carboxamide

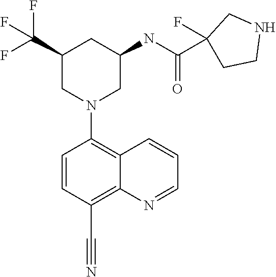 [SMILES: N#Cc1ccc(N2C[C@H](NC(=O)C3(F)CCNC3)C[C@H](C(F)(F)F)C2)c2cccnc12]

The title compound was prepared from (3R,5S)-1-(8-cyanoquinolin-5-yl)-5-(trifluoromethyl)piperidin-3-aminium trifluoroacetate and 1-[(tert-butoxy)carbonyl]-3-fluoropyrrolidine-3-carboxylic acid. MS: 436 [M+H]⁺. ¹H NMR (400 MHz, CDCl₃) δ 9.10 (dd, J=4.2, 1.7 Hz, 1H), 8.47 (dt, J=8.6, 2.0 Hz, 1H), 8.05 (d, J=7.9 Hz, 1H), 7.60 (ddd, J=8.6, 4.2, 1.1 Hz, 1H), 7.14 (d, J=7.9 Hz, 1H), 6.45 (t, J=6.9 Hz, 1H), 4.44-4.37 (m, 1H), 3.82-3.76 (m, 1H), 3.66-3.62 (m, 1H), 3.34-3.24 (m, 1H), 3.21-3.06 (m, 3H), 2.95 (t, J=11.3 Hz, 1H), 2.89-2.81 (m, 1H), 2.61-2.55 (td, J=11.2, 2.0 Hz, 1H), 2.51-2.46 (m, 1H), 2.45-2.24 (m, 1H), 2.21-2.02 (m, 1H), 1.57 (q, J=12.2 Hz, 1H).

### Examples 207: N-[(3R,5S)-1-(8-cyanoquinoxalin-5-yl)-5-methylpiperidin-3-yl]-2-(1,1-dioxo-1λ⁶-thietan-3-yl)acetamide

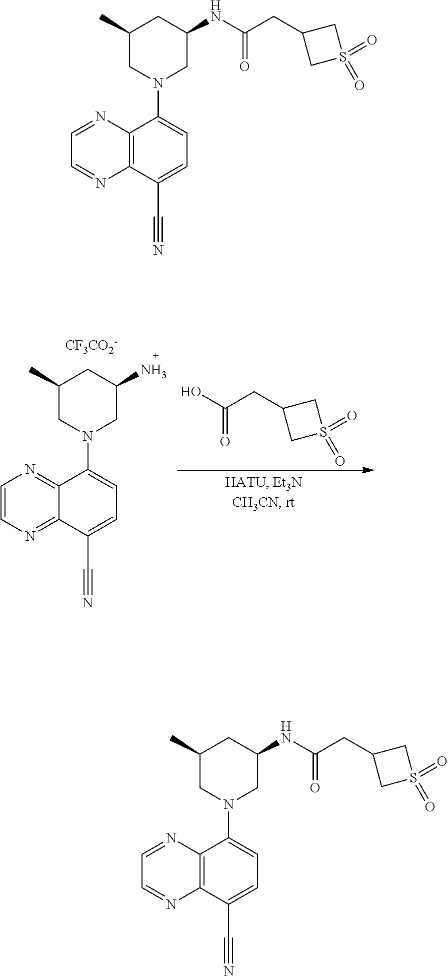 [SMILES: C[C@H]1C[C@@H](NC(=O)CC2CS(=O)(=O)C2)CN(c2ccc(C#N)c3nccnc23)C1 ; C[C@H]1C[C@@H](N)CN(c2ccc(C#N)c3nccnc23)C1 ; "CF3CO2-" ; O=C(O)CC1CS(=O)(=O)C1 ; C[C@H]1C[C@@H](NC(=O)CC2CS(=O)(=O)C2)CN(c2ccc(C#N)c3nccnc23)C1]

To a flask containing 8-[(3R,5S)-3-amino-5-methylpiperidin-1-yl]quinoxaline-5-carbonitrile; trifluoroacetic acid at room temperature was added DMF, 2-(1,1-dioxidothietan-3-yl)acetic acid (30.67 mg; 0.19 mmol; 1.50 eq.) and TEA (50.42 mg; 0.50 mmol; 4.0 eq.), followed by HATU (94.72 mg; 0.25 mmol; 2.0 eq.). The resulting mixture was stirred at room temperature for 1 h. The mixture was diluted with EtOAc, and filtered. The filtrated was washed with water and brine, dried, and concentrated. The crude was purified by prep-HPLC (ACN/water with 0.1% NH₄OH as modifier) to yield the title compound as white solid (46.1 mg, 89%). MS:414 [M+H]⁺.

¹H NMR (400 MHz, DMSO-d₆) δ 9.02 (d, J=1.8 Hz, 1H), 8.94 (d, J=1.8 Hz, 1H), 8.19 (d, J=8.5 Hz, 1H), 8.07 (d, J=7.4 Hz, 1H), 7.27 (d, J=8.5 Hz, 1H), 4.38-4.18 (m, 4H), 3.86 (dddd, J=10.4, 8.9, 6.2, 2.9 Hz, 3H), 2.86-2.60 (m, 3H), 2.57-2.45 (m, 6H), 1.15 (q, J=12.0 Hz, 1H), 0.91 (d, J=6.5 Hz, 3H).

The following compounds were synthesized in an analogous manner.

### Examples 208: (3S)—N-[(3R,5S)-1-(8-cyanoquinoxalin-5-yl)-5-methylpiperidin-3-yl]morpholine-3-carboxamide

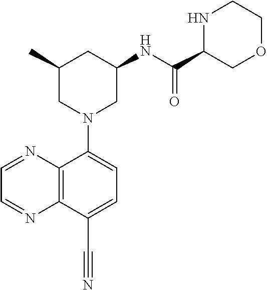 [SMILES: C[C@H]1C[C@@H](NC(=O)[C@@H]2COCCN2)CN(c2ccc(C#N)c3nccnc23)C1]

The title compound was prepared from 8-[(3R,5S)-3-amino-5-methylpiperidin-1-yl]quinoxaline-5-carbonitrile trifluoroacetic acid and (3S)-4-[(tert-butoxy)carbonyl]morpholine-3-carboxylic acid. MS: 381 [M+H]⁺. ¹H NMR (400 MHz, DMSO-d₆) δ 9.03 (d, J=1.8 Hz, 1H), 8.94 (d, J=1.8 Hz, 1H), 8.19 (d, J=8.4 Hz, 1H), 7.84 (d, J=7.5 Hz, 1H), 7.27 (d, J=8.5 Hz, 1H), 4.30 (d, J=12.5 Hz, 1H), 4.19 (d, J=11.9 Hz, 1H), 3.92 (d, J=11.7 Hz, 1H), 3.79-3.70 (m, 1H), 3.59 (d, J=11.2 Hz, 1H), 3.39 (q, J=10.8, 10.2 Hz, 2H), 2.88-2.75 (m, 2H), 2.74-2.63 (m, 2H), 1.91 (t, J=15.5 Hz, 2H), 1.24 (q, J=12.1 Hz, 1H), 0.92 (d, J=6.4 Hz, 3H).

### Examples 209: (3R)—N-[(3R,5S)-1-(8-cyanoquinoxalin-5-yl)-5-methylpiperidin-3-yl]morpholine-3-carboxamide

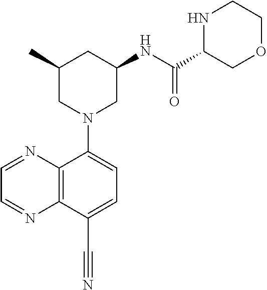 [SMILES: C[C@H]1C[C@@H](NC(=O)[C@H]2COCCN2)CN(c2ccc(C#N)c3nccnc23)C1]

The title compound was prepared from 8-[(3R,5S)-3-amino-5-methylpiperidin-1-yl]quinoxaline-5-carbonitrile trifluoroacetic acid and (3R)-4-[(tert-butoxy)carbonyl]morpholine-3-carboxylic acid. MS: 381 [M+H]⁺. ¹H NMR (400 MHz, DMSO-d₆) δ 9.03 (s, 1H), 8.94 (s, 1H), 8.20 (dd, J=8.6, 2.1 Hz, 1H), 7.80 (d, J=7.8 Hz, 1H), 7.27 (d, J=8.6 Hz, 1H), 4.30 (d, J=12.7 Hz, 1H), 4.19 (d, J=12.0 Hz, 1H), 3.91 (d, J=14.3 Hz, 1H), 3.73 (dt, J=11.1, 2.9 Hz, 1H), 3.64-3.54 (m, 1H), 3.40 (dt, J=19.5, 9.8 Hz, 2H), 3.28 (d, J=8.6 Hz, 2H), 2.93-2.62 (m, 5H), 1.94 (d, J=15.5 Hz, 2H), 1.26 (q, J=12.0 Hz, 1H), 0.93 (d, J=6.3 Hz, 3H).

### Examples 210: (3S)—N-[(3R,5S)-1-(8-cyanoquinoxalin-5-yl)-5-methylpiperidin-3-yl]-4-(dimethylamino)-3-hydroxybutanamide & Examples 211: (3R)—N-[(3R,5S)-1-(8-cyanoquinoxalin-5-yl)-5-methylpiperidin-3-yl]-4-(dimethylamino)-3-hydroxybutanamide

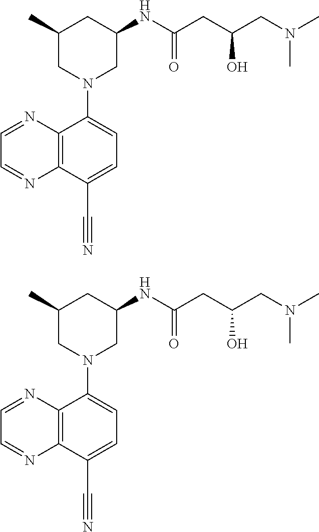

The title compounds were prepared from 8-[(3R,5S)-3-amino-5-methylpiperidin-1-yl]quinoxaline-5-carbonitrile trifluoroacetic acid and 4-(dimethylamino)-3-hydroxybutanoic acid, followed by chiral SFC separation under the following conditions: column, IA-H (4.6×100 mm), Prep SFC-P100; mobile phase, CO2/Methanol+20 mM NH4OH.

### Examples 210

MS: 397 [M+H]⁺. ¹H NMR (400 MHz, DMSO-d₆) δ 9.03 (s, 1H), 8.94 (s, 1H), 8.20 (d, J=8.4 Hz, 1H), 7.88 (d, J=7.5 Hz, 1H), 7.30 (d, J=8.5 Hz, 1H), 4.55 (d, J=4.5 Hz, 1H), 4.35 (d, J=13.1 Hz, 1H), 4.26 (d, J=12.5 Hz, 1H), 3.94 (s, 2H), 3.13-3.05 (m, 1H), 2.72 (dd, J=26.2, 10.7 Hz, 2H), 2.30-2.18 (m, 2H), 0.92 (d, J=6.4 Hz, 3H).

### Examples 211

MS: 397 [M+H]⁺. ¹H NMR (400 MHz, DMSO-d₆) δ 9.03 (s, 1H), 8.95 (s, 1H), 8.20 (d, J=8.3 Hz, 1H), 7.88 (d, J=7.5 Hz, 1H), 7.29 (d, J=8.4 Hz, 1H), 4.56 (d, J=4.1 Hz, 1H), 4.36 (d, J=12.7 Hz, 1H), 4.24 (d, J=12.0 Hz, 1H), 3.94 (s, 2H), 2.81-2.64 (m, 2H), 2.38-2.20 (m, 3H), 2.16 (d, J=2.1 Hz, 7H), 0.92 (d, J=6.5 Hz, 3H).

### Example 212: 8-[(2R,6R)-2-Methyl-6-(5-methyl-[1,3,4]oxadiazol-2-yl)-morpholin-4-yl]-quinoxaline-5-carbonitrile

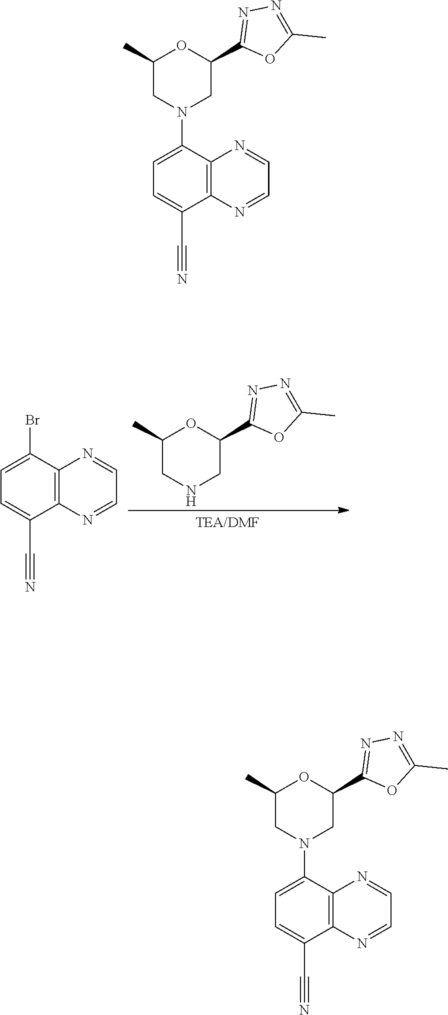

To a 25 mL microwave vial, was added 8-Bromo-quinoxaline-5-carbonitrile (90 mg; 0.38 mmol; 1.0 eq.), rac-(2r,6r)-2-methyl-6-(5-methyl-1,3,4-oxadiazol-2-yl)morpholine (84.54 mg; 0.46 mmol; 1.20 eq.), triethylamine (0.12 ml; 0.85 mmol; 2.20 eq.) and anhydrous DMF (1.0 ml). The tube was sealed and the yellow solution was microwaved at 125° C. for 3 h. The solid was collected by filtration of the reaction mixture and then washed with methanol and water to provide the title compound as a yellow solid (65 mg, yield: 50%). MS: 337 [M+H]⁺. 1H NMR (400 MHz, DMSO-d₆) δ 9.04 (dd, J=33.1, 1.8 Hz, 2H), 8.28 (d, J=8.3 Hz, 1H), 7.34 (d, J=8.4 Hz, 1H), 5.18 (dd, J=10.8, 2.5 Hz, 1H), 4.63 (dt, J=12.5, 2.3 Hz, 1H), 4.23-4.02 (m, 2H), 3.38 (dd, J=12.5, 10.8 Hz, 1H), 3.0-2.82 (m, 1H), 2.54 (s, 3H), 1.24 (d, J=6.1 Hz, 3H).

### Example 213: 8-[(2R,6R)-2-Methyl-6-(3-methyl-[1,2,4]oxadiazol-5-yl)-morpholin-4-yl]-quinoxaline-5-carbonitrile

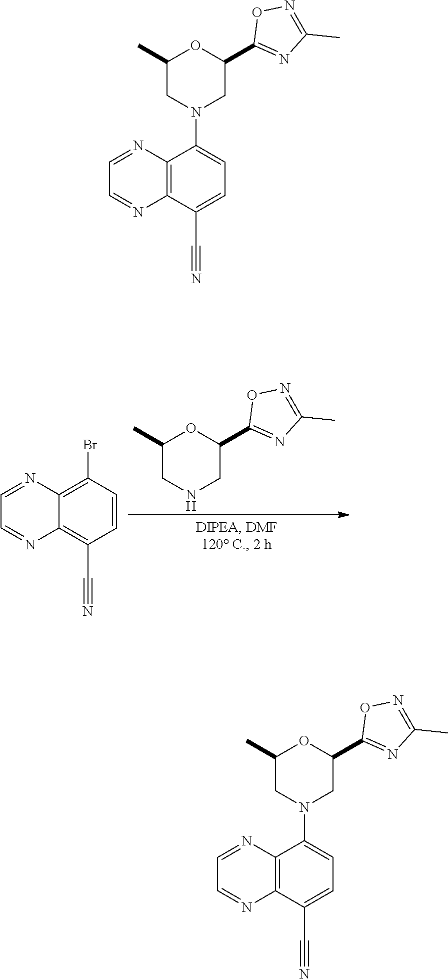

In a 25 mL microwave vial, rac-(2r,6r)-2-methyl-6-(3-methyl-1,2,4-oxadiazol-5-yl)morpholine hydrochloride (50.0 mg; 0.23 mmol; 1.0 eq.), 8-Bromo-quinoxaline-5-carbonitrile (53.27 mg; 0.23 mmol; 1.0 eq.) and DIEA (0.11 ml; 0.68 mmol; 3.0 eq.) were dissolved in anhydrous N,N-Dimethyl-formamide (2 ml). The tube was sealed and the yellow solution was microwaved at 120° C. for 2 h. The volatiles were evaporated and the residue dissolved in DCM (2 mL). The solution was absorbed on a PuriFlash 12 g column and purified by chromatography (Hexane-Ethyl acetate, gradient 80-20% for 5 minutes then 35-65% for 25 minutes. The desired fractions were combined and evaporated to give 8-[(2R,6R)-2-Methyl-6-(3-methyl-[1,2,4]oxadiazol-5-yl)-morpholin-4-yl]-quinoxaline-5-carbonitrile (47.0 mg; 0.14 mmol) as a yellow solid. MS: 337 [M+H]⁺. ¹H NMR (400 MHz, DMSO-d₆) δ 9.08 (d, J=1.8 Hz, 1H), 9.01 (d, J=1.8 Hz, 1H), 8.27 (d, J=8.3 Hz, 1H), 7.34 (d, J=8.4 Hz, 1H), 5.26 (dd, J=10.8, 2.6 Hz, 1H), 4.66 (dt, J=12.5, 2.2 Hz, 1H), 4.22-4.07 (m, 2H), 3.40-3.32 (m, 1H), 2.94 (dd, J=12.4, 10.2 Hz, 1H), 2.38 (s, 3H), 1.25 (d, J=6.1 Hz, 3H).

The following compounds were synthesized in an analogous manner.

### Example 214: 8-[(2R,6R)-2-(3-Cyclopropyl-[1,2,4]oxadiazol-5-yl)-6-methyl-morpholin-4-yl]-quinoxaline-5-carbonitrile

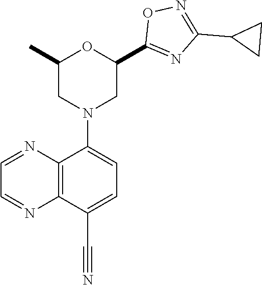 [SMILES: C[C@@H]1CN(c2ccc(C#N)c3nccnc23)C[C@H](c2nc(C3CC3)no2)O1]

The title compound was prepared from 8-Bromo-quinoxaline-5-carbonitrile and rac-(2r,6r)-2-(3-cyclopropyl-1,2,4-oxadiazol-5-yl)-6-methylmorpholine hydrochloride. MS:363 [M+H]⁺. ¹H NMR (400 MHz, Methanol-d₄) δ 8.98 (d, J=1.8 Hz, 1H), 8.93 (d, J=1.8 Hz, 1H), 8.16 (d, J=8.3 Hz, 1H), 7.30 (d, J=8.4 Hz, 1H), 5.26 (dd, J=10.7, 2.7 Hz, 1H), 4.61 (d, J=12.3 Hz, 1H), 4.27-4.08 (m, 2H), 3.31-3.22 (m, 1H), 2.94 (dd, J=12.2, 10.2 Hz, 1H), 2.14 (tt, J=8.3, 4.9 Hz, 1H), 1.15-1.06 (m, 2H), 1.06-0.92 (m, 2H).

### Example 215: 8-[(2S,6R)-2-(3,3-Difluoro-pyrrolidin-1-ylmethyl)-6-methyl-morpholin-4-yl]-quinoxaline-5-carbonitrile

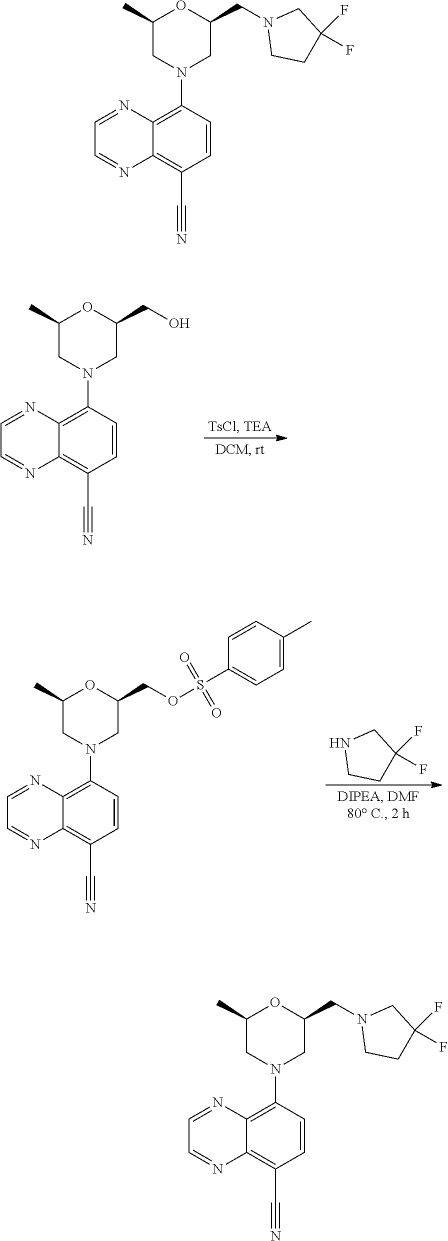 [SMILES: C[C@@H]1CN(c2ccc(C#N)c3nccnc23)C[C@H](CN2CCC(F)(F)C2)O1 ; C[C@@H]1CN(c2ccc(C#N)c3nccnc23)C[C@H](CO)O1 ; Cc1ccc(S(=O)(=O)OC[C@H]2CN(c3ccc(C#N)c4nccnc34)C[C@@H](C)O2)cc1 ; FC1(F)CCNC1 ; C[C@@H]1CN(c2ccc(C#N)c3nccnc23)C[C@H](CN2CCC(F)(F)C2)O1]

[(2R,6R)-4-(8-cyanoquinoxalin-5-yl)-6-methylmorpholin-2-yl]methyl 4-methylbenzene-1-sulfonate: Into a 20 mL schlenck reactor, was placed 8-[(2R,6R)-2-(hydroxymethyl)-6-methylmorpholin-4-yl]quinoxaline-5-carbonitrile (460.0 mg; 1.62 mmol; 1.0 eq.), DCM (10.0 ml), 4-methylbenzene-1-sulfonyl chloride (616.91 mg; 3.24 mmol; 2.0 eq.). This was followed by the addition of TEA (451.02 μl; 3.24 mmol; 2.0 eq.) with stirring at 20° C. The resulting solution was stirred for 3 h at 20° C. The reaction was then quenched by the addition of 20 mL of water. The resulting solution was extracted with 2×50 mL of DCM and the organic layers combined and dried over anhydrous sodium sulfate and concentrated under vacuum. The residue was purified by chromatography on a Biotage (PuriFlash Column, 15μ Si HP, 25 g); hexane/ethyl acetate, gradient from 80-20% to 20-80% for 15 minutes to give [(2R,6R)-4-(8-cyanoquinoxalin-5-yl)-6-methylmorpholin-2-yl]methyl 4-methylbenzene-1-sulfonate (630.0 mg; 89%) as a yellow solid. MS: 439 [M+H]⁺.

8-[(2S,6R)-2-(3,3-Difluoro-pyrrolidin-1-ylmethyl)-6-methyl-morpholin-4-yl]-quinoxaline-5-carbonitrile: Into a 25 mL vial was placed [(2R,6R)-4-(8-cyanoquinoxalin-5-yl)-6-methylmorpholin-2-yl]methyl 4-methylbenzene-1-sulfonate (45.0 mg; 0.10 mmol; 1.0 eq.), 3,3-difluoropyrrolidine hydrochloride (29.47 mg; 0.21 mmol; 2.0 eq.), MeCN (1.50 ml), TEA (44.63 μl; 0.32 mmol; 3.13 eq.). The resulting solution was stirred for 10 h at 80° C. The resulting mixture was concentrated under vacuum. The residue was purified by chromatography on a Biotage (PuriFlash Column, 15μ Si HP, 25 g) with ethyl acetate/petroleum ether (00:100 to 50:50) for 20 minutes. This resulted in 8-[(2S,6R)-2-(3,3-Difluoro-pyrrolidin-1-ylmethyl)-6-methyl-morpholin-4-yl]-quinoxaline-5-carbonitrile (5.70 mg; 15%) as a yellow solid. MS: 439 [M+H]⁺. ¹H NMR (400 MHz, Methanol-d₄) δ 8.96 (d, J=2.0 Hz, 1H), 8.88 (d, J=2.1 Hz, 1H), 8.13 (dd, J=8.5, 1.7 Hz, 1H), 7.22 (dd, J=8.2, 1.7 Hz, 1H), 4.32 (dt, J=12.3, 2.2 Hz, 1H), 4.17-3.93 (m, 3H), 3.12 (dd, J=13.8, 11.8 Hz, 1H), 2.98 (q, J=12.6 Hz, 1H), 2.88 (t, J=7.2 Hz, 2H), 2.82-2.63 (m, 4H), 2.28 (dt, J=15.0, 7.6 Hz, 2H), 1.27 (dd, J=6.2, 1.7 Hz, 3H).

The following compounds were synthesized in an analogous manner.

### Example 216: 8-[(2S,6R)-2-(3-Hydroxy-azetidin-1-ylmethyl)-6-methyl-morpholin-4-yl]-quinoxaline-5-carbonitrile

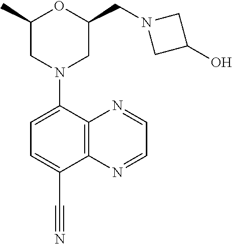 [SMILES: C[C@@H]1CN(c2ccc(C#N)c3nccnc23)C[C@H](CN2CC(O)C2)O1]

The title compound was prepared from [(2R,6R)-4-(8-cyanoquinoxalin-5-yl)-6-methylmorpholin-2-yl]methyl 4-methylbenzene-1-sulfonate and Azetidin-3-ol. MS: 340 [M+H]⁺. ¹H NMR (400 MHz, Chloroform-d) δ 9.0 (s, 1H), 8.86 (s, 1H), 8.04 (d, J=8.2 Hz, 1H), 7.08 (d, J=8.2 Hz, 1H), 4.54-4.43 (m, 1H), 4.12 (t, J=12.2 Hz, 2H), 4.06-3.89 (m, 2H), 3.79 (t, J=6.0 Hz, 2H), 3.09 (s, 2H), 2.86-2.68 (m, 4H), 2.09 (bs, 1H), 1.29 (d, J=6.6 Hz, 3H).

### Example 217: 8-[(2S,6R)-2-(4-Diethylamino-piperidin-1-ylmethyl)-6-methyl-morpholin-4-yl]-quinoxaline-5-carbonitrile

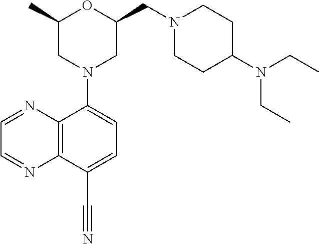 [SMILES: CCN(CC)C1CCN(C[C@H]2CN(c3ccc(C#N)c4nccnc34)C[C@@H](C)O2)CC1]

The title compound was prepared from [(2R,6R)-4-(8-cyanoquinoxalin-5-yl)-6-methylmorpholin-2-yl]methyl 4-methylbenzene-1-sulfonate and 4-diethylamino-piperidine. MS: 423 [M+H]⁺. ¹H NMR (400 MHz, Chloroform-d) δ 8.99 (s, 1H), 8.84 (s, 1H), 8.05 (d, J=7.9 Hz, 1H), 7.08 (d, J=8.0 Hz, 1H), 4.22 (d, J=11.9 Hz, 1H), 4.15-4.08 (m, 3H), 3.14 (d, J=11.0 Hz, 1H), 2.97 (d, J=10.8 Hz, 1H), 2.75 (t, J=11.0 Hz, 2H), 2.69-2.58 (m, 6H), 2.50 (d, J=6.2 Hz, 1H), 2.15 (t, J=11.5 Hz, 1H), 2.04 (t, J=11.4 Hz, 1H), 1.80 (bs, 2H), 1.67-1.57 (m, 2H), 1.29 (d, J=3.3 Hz, 3H), 1.10 (bs, 6H).

### Example 218: 8-[(2S,6R)-2-(3-Hydroxy-3-methyl-azetidin-1-ylmethyl)-6-methyl-morpholin-4-yl]-quinoxaline-5-carbonitrile

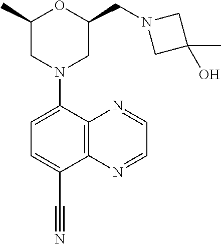

The title compound was prepared from [(2R,6R)-4-(8-cyanoquinoxalin-5-yl)-6-methylmorpholin-2-yl]methyl 4-methylbenzene-1-sulfonate and 3-methylazetidin-3-ol trifluoroacetic acid. MS: 354 [M+H]⁺. ¹H NMR (400 MHz, Methanol-d₄) δ 8.96 (d, J=1.8 Hz, 1H), 8.89 (d, J=1.8 Hz, 1H), 8.12 (d, J=8.3 Hz, 1H), 7.21 (d, J=8.4 Hz, 1H), 4.18 (ddt, J=24.1, 12.0, 2.2 Hz, 2H), 4.07-3.90 (m, 2H), 3.51-3.40 (m, 2H), 3.18 (dd, J=11.4, 7.9 Hz, 2H), 2.86-2.62 (m, 4H), 1.49 (s, 3H), 1.26 (d, J=6.3 Hz, 3H).

### Example 219: 8-[(2S,6R)-2-(4-Hydroxy-piperidin-1-ylmethyl)-6-methyl-morpholin-4-yl]-quinoxaline-5-carbonitrile

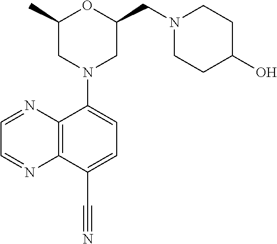 [SMILES: C[C@@H]1CN(c2ccc(C#N)c3nccnc23)C[C@H](CN2CCC(O)CC2)O1]

The title compound was prepared from [(2R,6R)-4-(8-cyanoquinoxalin-5-yl)-6-methylmorpholin-2-yl]methyl 4-methylbenzene-1-sulfonate and Piperidin-4-ol. MS:268 [M+H]⁺. ¹H NMR (400 MHz, Methanol-d₄) δ 8.96 (s, 1H), 8.89 (s, 1H), 8.12 (d, J=8.3 Hz, 1H), 7.22 (d, J=8.1 Hz, 1H), 4.32-4.19 (m, 2H), 4.17-4.01 (m, 2H), 3.84-3.72 (m, 1H), 3.28-3.10 (m, 2H), 2.93-2.60 (m, 6H), 2.02-1.92 (m, 2H), 1.79-1.66 (m, 2H), 1.29 (d, J=4.7 Hz, 3H).

### Example 220: 8-[(2S,6R)-2-((S)-3-Hydroxy-pyrrolidin-1-ylmethyl)-6-methyl-morpholin-4-yl]-quinoxaline-5-carbonitrile

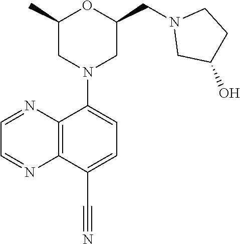 [SMILES: C[C@@H]1CN(c2ccc(C#N)c3nccnc23)C[C@H](CN2CC[C@H](O)C2)O1]

The title compound was prepared from [(2R,6R)-4-(8-cyanoquinoxalin-5-yl)-6-methylmorpholin-2-yl]methyl 4-methylbenzene-1-sulfonate and (s)-3-hydroxypyrrolidine. MS: 354 [M+H]⁺. ¹H NMR (400 MHz, Methanol-d₄) δ 8.98 (d, J=1.8 Hz, 1H), 8.91 (d, J=1.8 Hz, 1H), 8.16 (s, 1H), 7.26 (d, J=8.4 Hz, 1H), 4.55 (tt, J=5.0, 2.2 Hz, 1H), 4.33-4.23 (m, 2H), 4.17 (dt, J=12.1, 2.0 Hz, 1H), 4.14-4.05 (m, 1H), 3.70-3.38 (m, 3H), 3.30-3.17 (m, 3H), 2.82 (ddd, J=12.1, 10.5, 7.1 Hz, 2H), 2.27 (dtd, J=14.2, 8.6, 5.8 Hz, 1H), 2.05-1.94 (m, 1H), 1.33 (d, J=6.2 Hz, 3H).

### Example 221: 8-[(2S,6R)-2-((R)-3-Hydroxy-pyrrolidin-1-ylmethyl)-6-methyl-morpholin-4-yl]-quinoxaline-5-carbonitrile

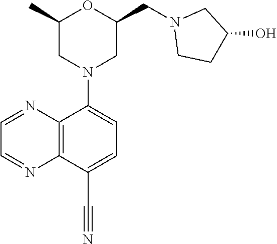 [SMILES: C[C@@H]1CN(c2ccc(C#N)c3nccnc23)C[C@H](CN2CC[C@@H](O)C2)O1]

The title compound was prepared from [(2R,6R)-4-(8-cyanoquinoxalin-5-yl)-6-methylmorpholin-2-yl]methyl 4-methylbenzene-1-sulfonate and (r)-3-hydroxypyrrolidine. MS: 354 [M+H]⁺. ¹H NMR (400 MHz, Methanol-d₄) δ 8.97 (d, J=1.8 Hz, 1H), 8.91 (d, J=1.8 Hz, 1H), 8.14 (d, J=8.3 Hz, 1H), 7.24 (d, J=8.4 Hz, 1H), 4.51 (tt, J=5.3, 2.6 Hz, 1H), 4.34-4.28 (m, 1H), 4.28-4.20 (m, 1H), 4.19-4.12 (m, 1H), 4.07 (ddq, J=12.5, 6.2, 3.1, 2.4 Hz, 1H), 3.32-3.12 (m, 5H), 3.08 (dd, J=12.9, 8.5 Hz, 1H), 2.81 (ddd, J=12.1, 10.4, 7.2 Hz, 2H), 2.32-2.20 (m, 1H), 1.98-1.86 (m, 1H), 1.31 (d, J=6.2 Hz, 3H).

### Example 222: 8-{(2S,6R)-2-[3-(1-Hydroxy-1-methyl-ethyl)-pyrrolidin-1-ylmethyl]-6-methyl-morpholin-4-yl}-quinoxaline-5-carbonitrile

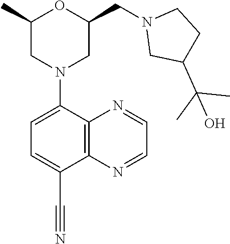 [SMILES: C[C@@H]1CN(c2ccc(C#N)c3nccnc23)C[C@H](CN2CCC(C(C)(C)O)C2)O1]

The title compound was prepared from [(2R,6R)-4-(8-cyanoquinoxalin-5-yl)-6-methylmorpholin-2-yl]methyl 4-methylbenzene-1-sulfonate and 2-(pyrrolidin-3-yl)propan-2-ol. MS: 396 [M+H]⁺. ¹H NMR (400 MHz, Methanol-d₄) δ 8.98 (d, J=1.6 Hz, 1H), 8.91 (d, J=1.7 Hz, 1H), 8.15 (d, J=8.3 Hz, 1H), 7.27 (d, J=8.3 Hz, 1H), 4.40-4.28 (m, 2H), 4.21-4.08 (m, 2H), 3.65-3.36 (m, 5H), 3.25 (q, J=7.3 Hz, 1H), 2.90-2.81 (m, 2H), 2.56 (m, 1H), 2.14 (qd, J=8.7, 5.8, 4.0 Hz, 2H), 1.37-1.33 (m, 3H), 1.30-1.25 (m, 6H).

### Example 223: 7-Fluoro-8-methyl-5-[(2R,6S)-2-methyl-6-(4-pyrrolidin-1-yl-piperidin-1-ylmethyl)-morpholin-4-yl]-quinoline

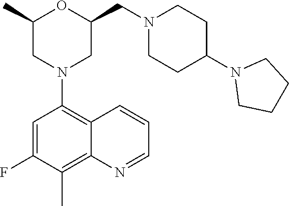 [SMILES: Cc1c(F)cc(N2C[C@H](CN3CCC(N4CCCC4)CC3)O[C@H](C)C2)c2cccnc12]

The title compound was prepared from [(2R,6R)-4-(7-Fluoro-8-methyl-quinolin-5-yl)-6-methyl-morpholin-2-yl]-methanol and 4-(1-pyrrolidinyl)piperidine. MS: 427 [M+H]⁺. ¹H NMR (400 MHz, Methanol-d₄) δ 8.88 (dd, J=4.3, 1.6 Hz, 1H), 8.62 (dd, J=8.5, 1.7 Hz, 1H), 7.49 (dd, J=8.5, 4.2 Hz, 1H), 7.05 (d, J=11.3 Hz, 1H), 4.17-3.99 (m, 2H), 3.29-3.11 (m, 3H), 3.03-2.95 (m, 1H), 2.81-2.32 (m, 11H), 2.22-2.04 (m, 3H), 1.95 (tt, J=9.9, 3.4 Hz, 2H), 1.88-1.74 (m, 4H), 1.59 (qd, J=12.3, 4.0 Hz, 2H), 1.24 (d, J=6.2 Hz, 3H).

### Example 224: 5-[(2R,6S)-2-Methyl-6-(4-methyl-piperazin-1-ylmethyl)-morpholin-4-yl]-quinazoline-8-carbonitrile

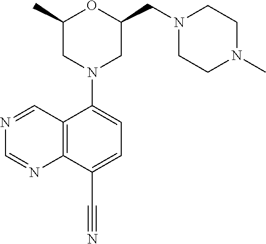 [SMILES: C[C@@H]1CN(c2ccc(C#N)c3ncncc23)C[C@H](CN2CCN(C)CC2)O1]

The title compound was prepared from 5-((2R,6R)-2-Hydroxymethyl-6-methyl-morpholin-4-yl)-quinazoline-8-carbonitrile and 1-Methyl-piperazine. MS: 367 [M+H]⁺. ¹H NMR (400 MHz, Methanol-d₄) δ 9.69 (s, 1H), 9.32 (s, 1H), 8.31 (s, 1H), 7.29 (d, J=8.2 Hz, 1H), 4.23-4.12 (m, 1H), 4.08 (dt, J=11.8, 6.4 Hz, 1H), 3.59 (dd, J=25.1, 12.1 Hz, 2H), 2.84 (dd, J=16.8, 11.4 Hz, 2H), 2.75-2.36 (m, 1OH), 2.30 (s, 3H), 1.27 (d, J=6.2 Hz, 3H).

### Example 225: 8-[(2R,6R)-2-(Azetidin-3-ylsulfanylmethyl)-6-methyl-morpholin-4-yl]-quinoxaline-5-carbonitrile hydrochloride

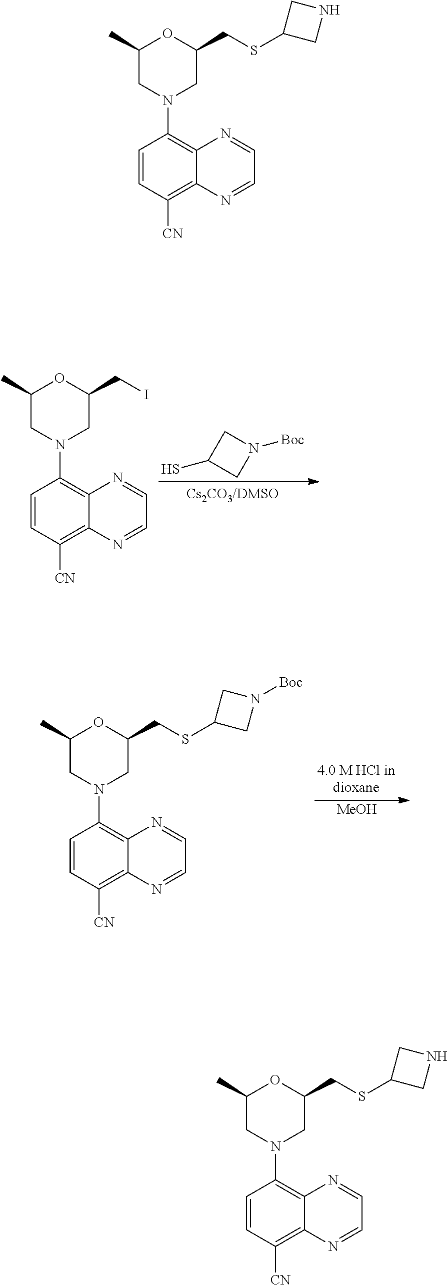

3-[(2R,6R)-4-(8-Cyano-quinoxalin-5-yl)-6-methyl-morpholin-2-ylmethylsulfanyl]-azetidine-1-carboxylic acid tert-butyl ester: A mixture of 8-((2R,6R)-2-iodomethyl-6-methyl-morpholin-4-yl)-quinoxaline-5-carbonitrile (400 mg; 1.01 mmol; 1.0 eq.), cesium carbonate (727 mg; 2.23 mmol; 2.20 eq.) and DMSO (4 ml) in a 20 ml microwave tube were stirred at 80° C. for 3 hr, until the reaction was completed. The reaction mixture was diluted with water (20 ml) and extracted with EA (30 ml). The organic layer was washed with brine, dried over Na₂SO₄ and concentrated. The residue was purified by silica column (50 g), eluting with 10-70% hexane/EA, to yield the title compound (400 mg, yield: 86.5%). MS:456 [M+H]⁺.

8-[(2R,6R)-2-(Azetidin-3-ylsulfanylmethyl)-6-methyl-morpholin-4-yl]-quinoxaline-5-carbonitrile hydrochloride (2): To a solution of 3-[(2R,6R)-4-(8-Cyano-quinoxalin-5-yl)-6-methyl-morpholin-2-ylmethylsulfanyl]-azetidine-1-carboxylic acid tert-butyl ester (400 mg; 0.88 mmol; 1.0 eq.) in methanol (5 ml) was added hydrogen chloride (4.0M in dioxane) (2.20 ml; 8.78 mmol; 10.0 eq.). The mixture was stirred at RT for 3 hrs, until the reaction was completed. The precipitate was filtered and dried to provide the title compound (370 mg, 98.4%). MS: 356 [M+H]⁺. ¹H NMR (400 MHz, Methanol-d₄) δ 8.94 (dd, J=26.3, 1.8 Hz, 2H), 8.14 (d, J=8.3 Hz, 1H), 7.23 (d, J=8.4 Hz, 1H), 4.45 (dtd, J=9.4, 6.4, 4.3 Hz, 2H), 4.30-4.12 (m, 3H), 4.09-3.93 (m, 4H), 3.68 (s, 5H), 2.98-2.64 (m, 4H), 1.29 (d, J=6.3 Hz, 3H).

### Example 226: 8-{(2R,6R)-2-[1-(2-Hydroxy-2-methyl-propyl)-azetidin-3-ylsulfanylmethyl]-6-methyl-morpholin-4-yl}-quinoxaline-5-carbonitrile

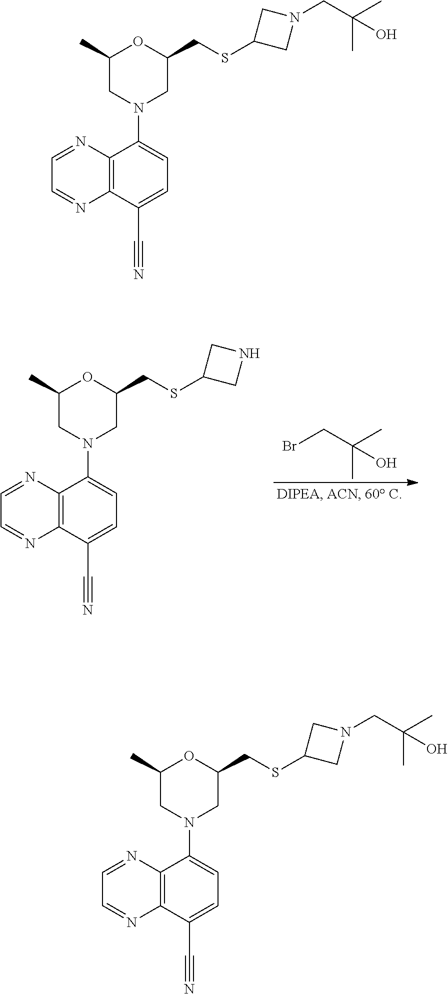

8-[(2R,6R)-2-(Azetidin-3-ylsulfanylmethyl)-6-methyl-morpholin-4-yl]-quinoxaline-5-carbonitrile hydrochloride (2) (50.0 mg; 0.12 mmol; 1.0 eq.), 1-Bromo-2-methyl-propan-2-ol (26.79 mg; 0.18 mmol; 1.50 eq.) and Ethyl-diisopropyl-amine (0.06 ml; 0.35 mmol; 3.0 eq.) were placed in acetonitrile (1 mL). The reaction mixture was stirred at 60° C. overnight. After the reaction was completed, the crude was purified by prep HPLC with an acetonitrile/water (0.1% NH₄OH modified) gradient to obtain the title compound (5.30 mg; 0.01 mmol; 10.6%). MS: 428.2 [M+H]⁺. 1H NMR (400 MHz, DMSO-d₆) δ 9.06 (d, J=1.8 Hz, 1H), 8.98 (d, J=1.8 Hz, 1H), 8.24 (d, J=8.4 Hz, 1H), 7.23 (d, J=8.4 Hz, 1H), 4.35 (d, J=12.3 Hz, 1H), 4.13 (d, J=12.3 Hz, 2H), 4.0 (s, 1H), 3.86 (s, 3H), 3.66 (q, J=6.5, 6.0 Hz, 2H), 3.60 (dd, J=13.8, 7.1 Hz, 2H), 3.03-2.94 (m, 2H), 2.84-2.65 (m, 6H), 2.30 (s, 2H), 1.18 (d, J=6.2 Hz, 3H), 1.01 (s, 6H).

### Example 227: 8-[(2R,6R)-2-(2,3-Dihydroxy-propylsulfanylmethyl)-6-methyl-morpholin-4-yl]-quinoxaline-5-carbonitrile

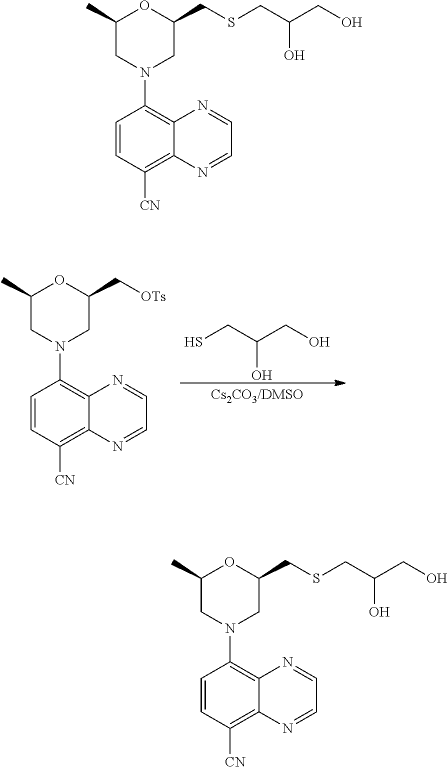 [SMILES: C[C@@H]1CN(c2ccc(C#N)c3nccnc23)C[C@H](CSCC(O)CO)O1 ; Cc1ccc(S(=O)(=O)OC[C@H]2CN(c3ccc(C#N)c4nccnc34)C[C@@H](C)O2)cc1 ; OCC(O)CS ; C[C@@H]1CN(c2ccc(C#N)c3nccnc23)C[C@H](CSCC(O)CO)O1]

A mixture of [(2R,6R)-4-(8-cyanoquinoxalin-5-yl)-6-methylmorpholin-2-yl]methyl 4-methylbenzene-1-sulfonate (55 mg; 0.13 mmol; 1.0 eq.), cesium carbonate (81 mg; 0.25 mmol; 2.0 eq.), 3-Mercapto-propane-1,2-diol (27 mg; 0.25 mmol; 2.0 eq.) and DMSO (1 ml) in a 10 ml microwave tube was stirred at 70° C. overnight. After the reaction was completed, the crude was purified by prep HPLC, eluting with 20-70% ACN/water (contained 0.1% ammonia), to yield the title compound (15 mg, yield 31%). MS:375 [M+H]⁺. ¹H NMR (400 MHz, DMSO-d₆) δ 9.06 (d, J=1.8 Hz, 1H), 9.04-8.92 (m, 1H), 8.24 (d, J=8.4 Hz, 1H), 7.24 (d, J=8.4 Hz, 1H), 4.77 (d, J=5.1 Hz, 1H), 4.57 (t, J=5.6 Hz, 1H), 4.43-4.31 (m, 1H), 4.23-4.10 (m, 1H), 3.90 (ddp, J=8.6, 6.4, 2.2 Hz, 2H), 3.60 (p, J=5.4 Hz, 1H), 3.37 (d, J=10.5 Hz, 2H), 2.84-2.63 (m, 4H), 2.63-2.51 (m, 2H), 1.18 (d, J=6.2 Hz, 3H).

The following compounds were synthesized in an analogous manner.

### Example 228: 1-[(2S,6R)-4-(8-Cyano-quinoxalin-5-yl)-6-methyl-morpholin-2-ylmethyl]-4-fluoro-piperidine-4-carboxylic acid

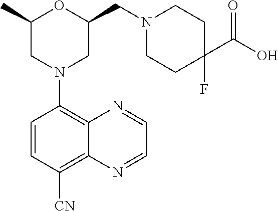 [SMILES: C[C@@H]1CN(c2ccc(C#N)c3nccnc23)C[C@H](CN2CCC(F)(C(=O)O)CC2)O1]

The title compound was prepared from [(2R,6R)-4-(8-cyanoquinoxalin-5-yl)-6-methylmorpholin-2-yl]methyl 4-methylbenzene-1-sulfonate and 4-Fluoro-piperidine-4-carboxylic acid hydrochloride (2). MS: 414 [M+H]⁺. ¹H NMR (400 MHz, Methanol-d₄) δ 8.95 (dd, J=30.3, 1.8 Hz, 2H), 8.17 (d, J=8.3 Hz, 1H), 7.27 (d, J=8.4 Hz, 1H), 4.42 (t, J=10.0 Hz, 1H), 4.31 (d, J=12.0 Hz, 1H), 4.23-4.02 (m, 2H), 3.63 (s, 2H), 2.85 (ddd, J=12.3, 10.3, 5.0 Hz, 2H), 2.44 (tt, J=23.6, 11.6 Hz, 2H), 2.26-2.02 (m, 2H), 1.35 (d, J=6.2 Hz, 3H).

### Example 229 (Isome 1): 8-[(2R,6R)-2-({[(2R)-2,3-dihydroxypropyl]sulfanyl}methyl)-6-methylmorpholin-4-yl]quinoxaline-5-carbonitrile & Example 230 (Isomer 2): 8-[(2R,6R)-2-({[(2S)-2,3-dihydroxypropyl]sulfanyl}methyl)-6-methylmorpholin-4-yl]quinoxaline-5-carbonitrile

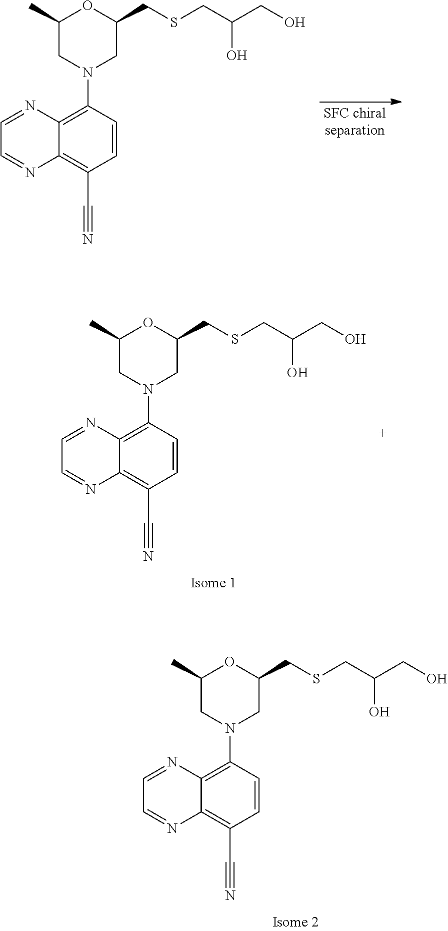 [SMILES: C[C@@H]1CN(c2ccc(C#N)c3nccnc23)C[C@H](CSCC(O)CO)O1 ; C[C@@H]1CN(c2ccc(C#N)c3nccnc23)C[C@H](CSCC(O)CO)O1 ; C[C@@H]1CN(c2ccc(C#N)c3nccnc23)C[C@H](CSCC(O)CO)O1]

The 2 isomers were obtained by separation of 8-[(2R,6R)-2-(2,3-Dihydroxy-propylsulfanylmethyl)-6-methyl-morpholin-4-yl]-quinoxaline-5-carbonitrile on chiral prep-HPLC under the following conditions: column, AS-H, Prep SFC-P100; mobile phase, methanol+20 Mm NH₄OH, 40° C./80 bar, 100 g/min; detector, PDA.

Isome 1: MS: 375.2 [M+H]⁺. 1H NMR (400 MHz, DMSO-d₆) δ 9.02 (dd, J=33.0, 1.8 Hz, 2H), 8.24 (d, J=8.4 Hz, 1H), 7.23 (d, J=8.5 Hz, 1H), 4.77 (d, J=5.0 Hz, 1H), 4.57 (t, J=5.6 Hz, 1H), 4.37 (d, J=12.3 Hz, 1H), 4.17 (d, J=12.3 Hz, 1H), 4.08 (q, J=5.2 Hz, 5H), 3.90 (dddd, J=10.5, 8.5, 6.0, 2.4 Hz, 2H), 3.60 (q, J=5.5 Hz, 1H), 3.36 (t, J=5.4 Hz, 3H), 3.17 (d, J=5.0 Hz, 12H), 2.84-2.80 (m, 1H), 2.80-2.71 (m, 3H), 2.68 (s, 1H), 1.17 (d, J=6.2 Hz, 3H).

Isomer 2: MS: 375.2 [M+H]⁺. 1 H NMR (400 MHz, DMSO-d₆) δ 9.02 (dd, J=33.0, 1.8 Hz, 2H), 8.24 (d, J=8.4 Hz, 1H), 7.23 (d, J=8.5 Hz, 1H), 4.77 (d, J=5.0 Hz, 1H), 4.57 (t, J=5.6 Hz, 1H), 4.37 (d, J=12.3 Hz, 1H), 4.17 (d, J=12.3 Hz, 1H), 4.08 (q, J=5.2 Hz, 5H), 3.90 (dddd, J=10.5, 8.5, 6.0, 2.4 Hz, 2H), 3.60 (q, J=5.5 Hz, 1H), 3.36 (t, J=5.4 Hz, 3H), 3.17 (d, J=5.0 Hz, 12H), 2.84-2.80 (m, 1H), 2.80-2.71 (m, 3H), 2.68 (s, 1H), 1.17 (d, J=6.2 Hz, 3H).

### Example 231: 8-[(2S,6S)-2-Methyl-6-(4-methyl-piperazin-1-ylmethyl)-morpholin-4-yl]-quinoxaline-5-carbonitrile

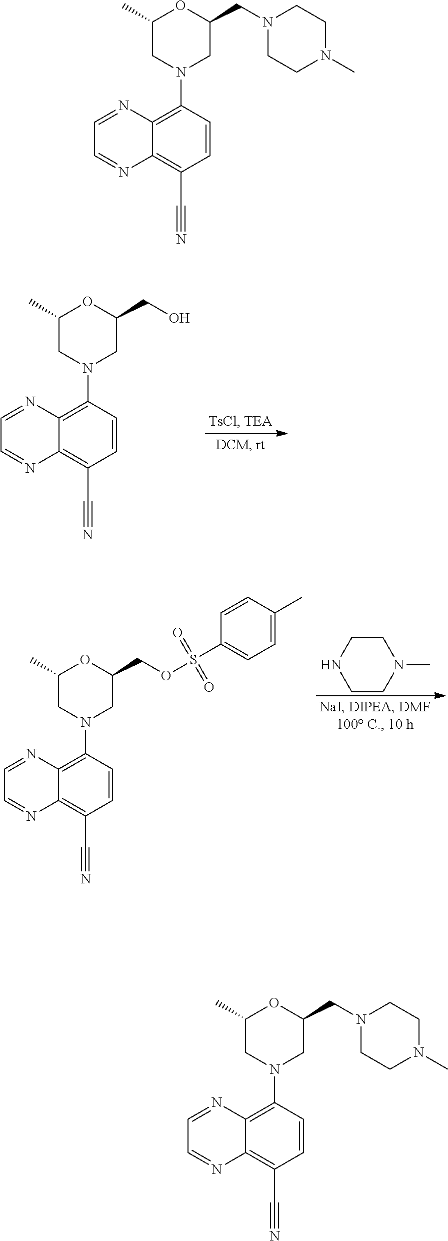

Toluene-4-sulfonic acid (2R,6S)-4-(8-cyano-quinoxalin-5-yl)-6-methyl-morpholin-2-ylmethyl ester: Into a 20 mL schlenck reactor, was placed 8-((2R,6S)-2-Hydroxymethyl-6-methyl-morpholin-4-yl)-quinoxaline-5-carbonitrile (50.0 mg; 0.18 mmol; 1.0 eq.), DCM (5.0 ml), 4-methylbenzene-1-sulfonyl chloride (67.06 mg; 0.35 mmol; 2.0 eq.). This was followed by the addition of TEA (49.02 μl; 0.35 mmol; 2.0 eq.) with stirring at 20° C. The resulting solution was stirred for 3 h at 20° C. The crude was loaded on a PuriFlash Column and purified by chromatography on a Biotage (PuriFlash Column, 15μ Si HP, 25 g); hexane/ethyl acetate, gradient from 80-20% to 20-80% for 15 minutes to give the title compound (62.0 mg; 80%) as a yellow solid. MS:439 [M+H]⁺.

8-[(2S,6S)-2-Methyl-6-(4-methyl-piperazin-1-ylmethyl)-morpholin-4-yl]-quinoxaline-5-carbonitrile: Into a 25 mL vial was placed toluene-4-sulfonic acid (2R,6S)-4-(8-cyano-quinoxalin-5-yl)-6-methyl-morpholin-2-ylmethyl ester (30.0 mg; 0.07 mmol; 1.0 eq.), 1-methylpiperazine (7.54 mg; 0.08 mmol; 1.10 eq.), sodium iodide (15.38 mg; 0.10 mmol; 1.50 eq.), MeCN (1.50 ml) and TEA (29.76 μl; 0.21 mmol; 3.13 eq.). The reaction solution was stirred for 10 h at 100° C. The resulting mixture was concentrated under vacuum. The residue was applied onto a silica gel column with ethyl acetate/petroleum ether (10:90-100:00) then MeOH/dichloromethane 5:90 for 10 minutes to yield the title compound (6.60 mg; 26%) as an orange gum. MS: 367 [M+H]⁺. ¹H NMR (400 MHz, Methanol-d₄) δ 8.95 (s, 1H), 8.88 (s, 1H), 8.12 (d, J=8.4 Hz, 1H), 7.21 (d, J=8.4 Hz, 1H), 4.31-4.23 (m, 2H), 4.16-3.98 (m, 1H), 3.82-3.70 (m, 2H), 3.61 (dd, J=12.3, 3.1 Hz, 1H), 3.40-3.36 (m, 1H), 2.90-2.73 (m, 3H), 2.62-2.51 (m, 4H), 1.36 (d, J=6.3 Hz, 3H).

The following compounds were synthesized in an analogous manner.

### Example 232: 8-[(2S,6S)-2-Methyl-6-(4-pyrrolidin-1-yl-piperidin-1-ylmethyl)-morpholin-4-yl]-quinoxaline-5-carbonitrile

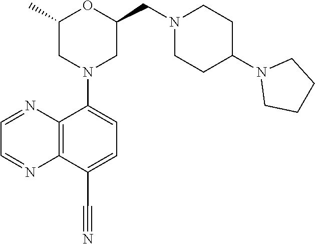 [SMILES: C[C@H]1CN(c2ccc(C#N)c3nccnc23)C[C@H](CN2CCC(N3CCCC3)CC2)O1]

The title compound was prepared from toluene-4-sulfonic acid (2R,6S)-4-(8-cyano-quinoxalin-5-yl)-6-methyl-morpholin-2-ylmethyl ester and 4-(1-pyrrolidinyl)piperidine. MS: 421 [M+H]⁺. ¹H NMR (400 MHz, Methanol-d₄) δ 8.95 (d, J=1.8 Hz, 1H), 8.88 (d, J=1.8 Hz, 1H), 8.12 (d, J=8.4 Hz, 1H), 7.21 (d, J=8.4 Hz, 1H), 4.25 (tq, J=6.5, 3.9, 3.5 Hz, 2H), 3.83-3.68 (m, 2H), 3.62 (dd, J=12.3, 3.4 Hz, 1H), 3.40-3.35 (m, 1H), 3.13 (s, 1H), 3.05-2.92 (m, 1H), 2.82-2.74 (m, 2H), 2.67 (d, J=5.8 Hz, 4H), 2.15 (ddd, J=26.6, 12.0, 2.6 Hz, 3H), 1.96 (d, J=12.6 Hz, 2H), 1.84 (p, J=3.2 Hz, 4H), 1.60 (ddt, J=19.3, 12.7, 6.9 Hz, 2H), 1.35 (d, J=6.4 Hz, 3H).

### Example 233: (2R,6R)-4-(8-Cyano-quinoxalin-5-yl)-6-methyl-morpholine-2-carboxylic acid ((R)-2,3-dihydroxy-propyl)-amide

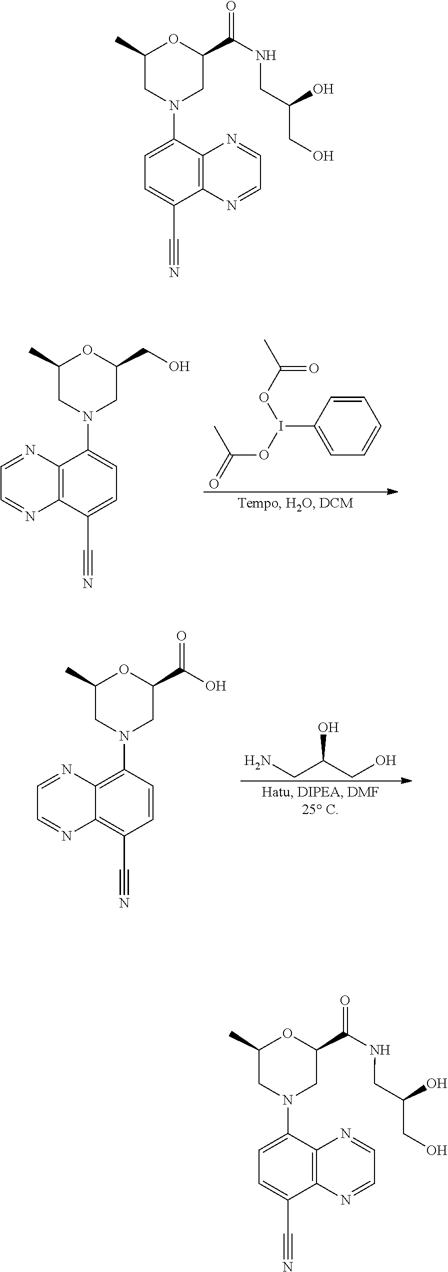 [SMILES: C[C@@H]1CN(c2ccc(C#N)c3nccnc23)C[C@H](C(=O)NC[C@@H](O)CO)O1 ; C[C@@H]1CN(c2ccc(C#N)c3nccnc23)C[C@H](CO)O1 ; CC(=O)OI(OC(C)=O)c1ccccc1 ; C[C@@H]1CN(c2ccc(C#N)c3nccnc23)C[C@H](C(=O)O)O1 ; NC[C@@H](O)CO ; C[C@@H]1CN(c2ccc(C#N)c3nccnc23)C[C@H](C(=O)NC[C@@H](O)CO)O1]

(2R,6R)-4-(8-Cyano-quinoxalin-5-yl)-6-methyl-morpholine-2-carboxylic acid: Into a 50-mL round-bottom flask, was placed 8-((2R,6R)-2-Hydroxymethyl-6-methyl-morpholin-4-yl)-quinoxaline-5-carbonitrile (1800.0 mg; 6.33 mmol; 1.0 eq.) and DCM (15.0 ml), the resulting solution was stirred for 5 minutes at 0° C. in a water/ice bath, then (diacetoxyiodo)benzene (4.08 g; 12.66 mmol; 2.0 eq.) was added. After raising the temperature to 10° C., tempo (197.84 mg; 1.27 mmol; 0.20 eq.) and water (0.80 ml) were added respectively. The resulting solution was stirred for an additional 20 minutes while the temperature was maintained at 10° C. in a water/ice bath. The reaction solution was stirring, for an additional 2 h at 25° C. after which the yellow solids suspension become a brown solution. LC/MS showed the reaction was complete. The reaction was then quenched by the addition of 0.5 mL of 10% sodium thiosulfate(aq), and was stirred for another 45 minutes. The resulting mixture was concentrated under vacuum. The residue was dispersed in a mixture of 1:1 DCM/methanol, filtered through celite and the filtrate evaporated to give (2R,6R)-4-(8-Cyano-quinoxalin-5-yl)-6-methyl-morpholine-2-carboxylic acid (2100.0 mg; crude) as a yellow solid. It was carried to the next step without further purification. MS: 299 [M+H]⁺.

(2R,6R)-4-(8-Cyano-quinoxalin-5-yl)-6-methyl-morpholine-2-carboxylic acid ((R)-2,3-dihydroxy-propyl)-amide: Into a 50 mL round-bottom flask, was placed (2R,6R)-4-(8-Cyano-quinoxalin-5-yl)-6-methyl-morpholine-2-carboxylic acid (150.0 mg; 0.45 mmol; 1.0 eq.) in DMF (2.0 ml), hatu (258.24 mg; 0.68 mmol; 1.50 eq.) was added and the resulting solution stirred for 10 minutes at rt after which (r)-3-amino-1,2-propanediol (61 mg; 0.68 mmol; 1.50 eq.) and DIPEA (0.25 ml; 1.3 mmol; 3.0 eq.) were respectively added. The resulting mixture was stirred at room temperature for 2 h. 3 mL of DMSO was added and the product was purified on waters reverse phase system using a gradient of 05-95% CH₃CN/H₂O (0.1% Ammonium Hydroxide) in 4 injections of 1.25 mL each. The desired fractions were evaporated to provide the title compound (82.0 mg; 49%) as a yellow solid. MS: 372 [M+H]⁺. ¹H NMR (400 MHz, Chloroform-d) δ 9.01 (d, J=1.6 Hz, 1H), 8.90 (d, J=1.6 Hz, 1H), 8.06 (d, J=8.2 Hz, 1H), 7.19-7.05 (m, 2H), 4.56-4.44 (m, 2H), 4.23-4.10 (m, 2H), 3.86 (p, J=5.0 Hz, 1H), 3.68-3.44 (m, 4H), 2.93 (t, J=12.0 Hz, 1H), 2.77 (d, J=10.4 Hz, 1H), 1.59 (s, 2H), 1.36 (d, J=6.2 Hz, 3H).

The following compounds were synthesized in an analogous manner.

### Example 234: (2R,6R)-4-(8-Cyano-quinoxalin-5-yl)-6-methyl-morpholine-2-carboxylic acid ((S)-2,3-dihydroxy-propyl)-amide

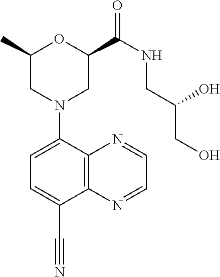 [SMILES: C[C@@H]1CN(c2ccc(C#N)c3nccnc23)C[C@H](C(=O)NC[C@H](O)CO)O1]

The title compound was prepared from (2R,6R)-4-(8-Cyano-quinoxalin-5-yl)-6-methyl-morpholine-2-carboxylic acid and (s)-3-amino-1,2-propanediol. MS: 372 [M+H]⁺. ¹H NMR (400 MHz, Methanol-d₄) δ 8.97 (d, J=1.9 Hz, 1H), 8.93 (d, J=1.9 Hz, 1H), 8.14 (d, J=8.3 Hz, 1H), 7.26 (d, J=8.4 Hz, 1H), 4.57 (dq, J=12.5, 2.8 Hz, 1H), 4.46 (dd, J=10.7, 2.8 Hz, 1H), 4.21-4.06 (m, 2H), 3.75 (dd, J=6.6, 4.7 Hz, 1H), 3.50 (dd, J=29.4, 5.0 Hz, 2H), 3.39-3.25 (m, 2H), 2.93 (t, J=11.5 Hz, 1H), 2.83 (dd, J=12.2, 10.1 Hz, 1H), 1.36 (d, J=6.1 Hz, 3H).

### Example 235: 8-[(2R,6R)-2-(3-Hydroxy-3-methyl-[1,3′]biazetidinyl-1′-carbonyl)-6-methyl-morpholin-4-yl]-quinoxaline-5-carbonitrile

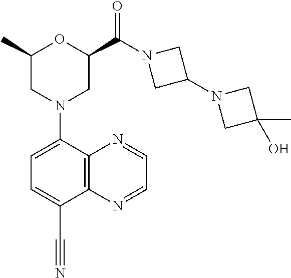

The title compound was prepared from (2R,6R)-4-(8-Cyano-quinoxalin-5-yl)-6-methyl-morpholine-2-carboxylic acid and 1-(azetidin-3-yl)-3-methylazetidin-3-ol dihydrochloride. MS: 423 [M+H]⁺. ¹H NMR (400 MHz, Methanol-d₄) δ 8.98 (d, J=1.7 Hz, 1H), 8.92 (d, J=2.5 Hz, 1H), 8.15 (d, J=8.3 Hz, 1H), 7.26 (d, J=8.3 Hz, 1H), 4.61-4.43 (m, 3H), 4.29-4.18 (m, 1H), 4.06 (ddd, J=20.6, 11.1, 7.6 Hz, 3H), 3.81 (d, J=10.8 Hz, 1H), 3.59 (dq, J=7.2, 3.7, 3.0 Hz, 1H), 3.36 (d, J=6.5 Hz, 2H), 3.14 (d, J=7.8 Hz, 2H), 3.08-2.99 (m, 1H), 2.87-2.79 (m, 1H), 1.51 (s, 3H), 1.32 (d, J=6.2 Hz, 3H).

### Example 236: (2R,6R)-4-(8-Cyano-quinoxalin-5-yl)-6-methyl-morpholine-2-carboxylic acid (2,6-dioxo-piperidin-3-yl)-amide

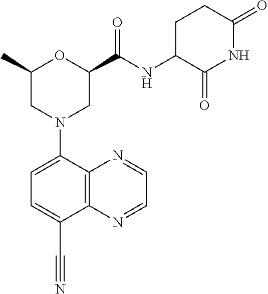 [SMILES: C[C@@H]1CN(c2ccc(C#N)c3nccnc23)C[C@H](C(=O)NC2CCC(=O)NC2=O)O1]

The title compound was prepared from (2R,6R)-4-(8-Cyano-quinoxalin-5-yl)-6-methyl-morpholine-2-carboxylic acid 3-aminopiperidine-2,6-dione. MS: 409 [M+H]⁺. ¹H NMR (400 MHz, Chloroform-d/MeOD) δ 8.92 (d, J=4.1 Hz, 1H), 8.84 (d, J=4.1 Hz, 1H), 8.01 (d, J=9.4 Hz, 1H), 7.58-7.43 (m, 1H), 7.08 (d, J=5.6 Hz, 1H), 4.55 (dt, J=12.5, 6.1 Hz, 1H), 4.48-4.31 (m, 2H), 4.16-4.03 (m, 2H), 2.98-2.81 (m, 1H), 2.81-2.58 (m, 3H), 2.47-2.34 (m, 1H), 1.99-1.81 (m, 1H), 1.30 (d, J=5.9 Hz, 3H).

### Example 237: (2R,6R)-4-(8-Cyano-quinoxalin-5-yl)-6-methyl-morpholine-2-carboxylic acid (3,3,3-trifluoro-2-hydroxy-propyl)-amide

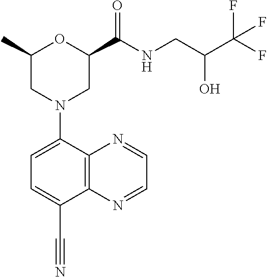 [SMILES: C[C@@H]1CN(c2ccc(C#N)c3nccnc23)C[C@H](C(=O)NCC(O)C(F)(F)F)O1]

The title compound was prepared from (2R,6R)-4-(8-Cyano-quinoxalin-5-yl)-6-methyl-morpholine-2-carboxylic acid and 3-amino-1,1,1-trifluoropropan-2-ol. MS: 410 [M+H]⁺. ¹H NMR (400 MHz, Methanol-d₄) δ 8.98 (d, J=1.8 Hz, 1H), 8.94 (d, J=1.8 Hz, 1H), 8.16 (d, J=8.3 Hz, 1H), 7.27 (d, J=8.3 Hz, 1H), 4.58 (dq, J=12.1, 2.3 Hz, 1H), 4.47 (dd, J=10.7, 2.8 Hz, 1H), 4.25-4.01 (m, 3H), 3.67 (ddd, J=13.8, 4.1, 1.9 Hz, 1H), 3.40 (dd, J=13.9, 8.2 Hz, 1H), 3.04 (s, 1H), 2.98-2.77 (m, 2H), 1.37 (d, J=6.2 Hz, 3H).

### Example 238: (2R,6R)-4-(8-Cyano-quinoxalin-5-yl)-6-methyl-morpholine-2-carboxylic acid ((R)-2-hydroxy-propyl)-amide

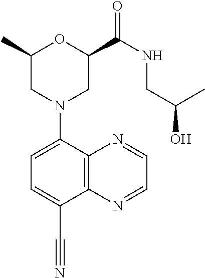 [SMILES: C[C@@H]1CN(c2ccc(C#N)c3nccnc23)C[C@H](C(=O)NC[C@@H](C)O)O1]

The title compound was prepared from (2R,6R)-4-(8-Cyano-quinoxalin-5-yl)-6-methyl-morpholine-2-carboxylic acid and (r)-(−)-1-amino-2-propanol. MS: 356 [M+H]⁺. ¹H NMR (400 MHz, Methanol-d₄) δ 8.98 (d, J=1.8 Hz, 1H), 8.94 (d, J=1.8 Hz, 1H), 8.15 (d, J=8.3 Hz, 1H), 7.27 (d, J=8.3 Hz, 1H), 4.58 (dt, J=12.2, 2.4 Hz, 1H), 4.46 (dd, J=10.8, 2.8 Hz, 1H), 4.22-4.02 (m, 2H), 3.94-3.80 (m, 1H), 3.44-3.34 (m, 1H), 3.20 (dd, J=13.5, 7.2 Hz, 1H), 2.93 (dd, J=12.2, 10.8 Hz, 1H), 2.83 (dd, J=12.2, 10.2 Hz, 1H), 1.36 (d, J=6.2 Hz, 3H), 1.18 (d, J=6.3 Hz, 3H).

### Example 239: (2R,6R)-4-(8-Cyano-quinoxalin-5-yl)-6-methyl-morpholine-2-carboxylic acid [2-(1,1-dioxo-1lambda6-thiomorpholin-4-yl)-ethyl]-amide

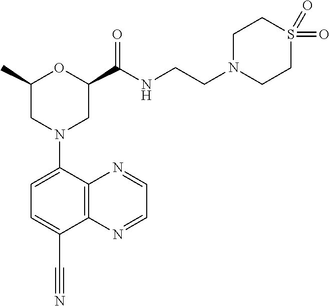 [SMILES: C[C@@H]1CN(c2ccc(C#N)c3nccnc23)C[C@H](C(=O)NCCN2CCS(=O)(=O)CC2)O1]

The title compound was prepared from (2R,6R)-4-(8-Cyano-quinoxalin-5-yl)-6-methyl-morpholine-2-carboxylic acid and 4-(2-aminoethyl)thiomorpholine 1,1-dioxide. MS: 459 [M+H]⁺. ¹H NMR (400 MHz, Methanol-d₄) δ 8.97 (s, 1H), 8.93 (s, 1H), 8.14 (d, J=8.3 Hz, 1H), 7.25 (d, J=8.3 Hz, 1H), 4.58 (d, J=12.3 Hz, 1H), 4.44 (d, J=10.7 Hz, 1H), 4.19-4.06 (m, 2H), 3.41 (t, J=6.5 Hz, 2H), 3.10 (t, J=7.0 Hz, 8H), 2.92 (t, J=11.5 Hz, 1H), 2.83 (t, J=11.2 Hz, 1H), 2.71 (t, J=6.5 Hz, 2H), 1.37 (d, J=6.2 Hz, 3H).

### Example 240: (2R,6R)-4-(8-Cyano-quinoxalin-5-yl)-6-methyl-morpholine-2-carboxylic acid (4-methyl-morpholin-2-ylmethyl)-amide

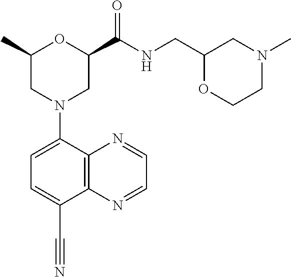

The title compound was prepared from (2R,6R)-4-(8-Cyano-quinoxalin-5-yl)-6-methyl-morpholine-2-carboxylic acid and (4-methylmorpholin-2-yl)methanamine. MS: 411 [M+H]⁺. ¹H NMR (400 MHz, Methanol-d₄) δ 8.97 (s, 1H), 8.93 (s, 1H), 8.14 (d, J=8.3 Hz, 1H), 7.25 (d, J=8.3 Hz, 1H), 4.56 (d, J=12.3 Hz, 1H), 4.44 (dt, J=10.5, 2.0 Hz, 1H), 4.16-4.04 (m, 2H), 3.93-3.87 (m, 1H), 3.64 (t, J=11.1 Hz, 2H), 3.40 (dt, J=12.4, 5.8 Hz, 1H), 3.29 (d, J=6.6 Hz, 1H), 2.86 (dq, J=34.0, 11.6 Hz, 3H), 2.70 (d, J=11.7 Hz, 1H), 2.30 (s, 3H), 2.15 (td, J=11.6, 3.3 Hz, 1H), 1.88 (td, J=10.9, 3.8 Hz, 1H), 1.36 (d, J=6.1 Hz, 3H).

### Example 241: (2R,6R)-4-(8-Cyano-quinoxalin-5-yl)-6-methyl-morpholine-2-carboxylic acid (1-cyclopropylmethyl-pyrrolidin-3-yl)-amide

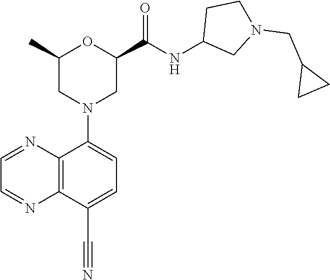 [SMILES: C[C@@H]1CN(c2ccc(C#N)c3nccnc23)C[C@H](C(=O)NC2CCN(CC3CC3)C2)O1]

The title compound was prepared from (2R,6R)-4-(8-Cyano-quinoxalin-5-yl)-6-methyl-morpholine-2-carboxylic acid and 1-(cyclopropylmethyl)pyrrolidin-3-amine. MS: 421 [M+H]⁺. ¹H NMR (400 MHz, Methanol-d₄) δ 8.97 (s, 1H), 8.92 (d, J=2.0 Hz, 1H), 8.13 (dd, J=8.3, 1.7 Hz, 1H), 7.33-7.13 (m, 1H), 4.61-4.47 (m, 2H), 4.42 (dt, J=10.8, 2.2 Hz, 1H), 4.20-3.99 (m, 2H), 3.08-2.66 (m, 5H), 2.53-2.42 (m, 1H), 2.39-2.25 (m, 3H), 1.73 (dt, J=13.7, 6.8 Hz, 1H), 1.37 (dd, J=6.2, 1.7 Hz, 3H), 0.94 (d, J=7.8 Hz, 1H), 0.64-0.47 (m, 2H), 0.28-0.11 (m, 2H).

### Example 242: (2R,6R)-4-(8-Cyano-quinoxalin-5-yl)-6-methyl-morpholine-2-carboxylic acid (1-acetyl-piperidin-4-yl)-amide

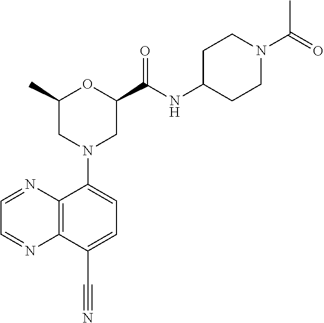 [SMILES: CC(=O)N1CCC(NC(=O)[C@H]2CN(c3ccc(C#N)c4nccnc34)C[C@@H](C)O2)CC1]

The title compound was prepared from (2R,6R)-4-(8-Cyano-quinoxalin-5-yl)-6-methyl-morpholine-2-carboxylic acid and 1-acetylpiperidin-4-amine. MS: 423 [M+H]⁺. ¹H NMR (400 MHz, Methanol-d₄) δ 8.97 (d, J=1.5 Hz, 1H), 8.93 (d, J=1.4 Hz, 1H), 8.14 (d, J=8.3 Hz, 1H), 7.25 (d, J=8.3 Hz, 1H), 4.55 (d, J=12.0 Hz, 2H), 4.46-4.39 (m, 1H), 4.23-3.88 (m, 4H), 3.23 (t, J=12.9 Hz, 1H), 2.98-2.71 (m, 3H), 2.13 (s, 3H), 2.01-1.83 (m, 2H), 1.65-1.43 (m, 2H), 1.35 (d, J=6.1 Hz, 3H).

### Example 243: (2R,6R)-4-(8-Cyano-quinoxalin-5-yl)-6-methyl-morpholine-2-carboxylic acid (2-acetylamino-ethyl)-amide

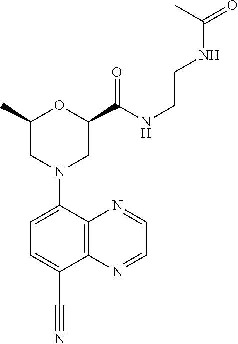 [SMILES: CC(=O)NCCNC(=O)[C@H]1CN(c2ccc(C#N)c3nccnc23)C[C@@H](C)O1]

The title compound was prepared from (2R,6R)-4-(8-Cyano-quinoxalin-5-yl)-6-methyl-morpholine-2-carboxylic acid and N-(2-aminoethyl)acetamide. MS: 383 [M+H]⁺. ¹H NMR (400 MHz, Methanol-d₄) δ 8.97 (s, 1H), 8.93 (s, 1H), 8.14 (d, J=8.3 Hz, 1H), 7.25 (d, J=8.3 Hz, 1H), 4.55 (d, J=12.2 Hz, 1H), 4.42 (dd, J=10.9, 2.7 Hz, 1H), 4.25-4.03 (m, 2H), 3.44-3.35 (m, 3H), 3.01 (bs, 1H), 2.92 (t, J=11.5 Hz, 1H), 2.82 (t, J=11.3 Hz, 1H), 1.96 (s, 3H), 1.36 (d, J=6.2 Hz, 3H).

### Example 244: (2R,6R)-4-(8-Cyano-quinoxalin-5-yl)-6-methyl-morpholine-2-carboxylic acid [2-(ethyl-methyl-amino)-ethyl]-amide

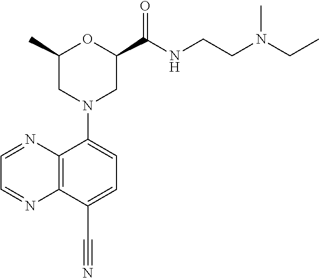 [SMILES: CCN(C)CCNC(=O)[C@H]1CN(c2ccc(C#N)c3nccnc23)C[C@@H](C)O1]

The title compound was prepared from (2R,6R)-4-(8-Cyano-quinoxalin-5-yl)-6-methyl-morpholine-2-carboxylic acid and (2-aminoethyl)(ethyl)methylamine. MS: 383 [M+H]⁺. ¹H NMR (400 MHz, Methanol-d₄) δ 8.96 (d, J=1.6 Hz, 1H), 8.93 (d, J=1.6 Hz, 1H), 8.13 (dd, J=8.3, 1.3 Hz, 1H), 7.30-7.12 (m, 1H), 4.56 (dd, J=12.0, 2.4 Hz, 1H), 4.42 (dt, J=10.7, 2.1 Hz, 1H), 4.15 (dd, J=12.3, 2.1 Hz, 1H), 4.12-4.04 (m, 1H), 3.41 (q, J=6.4 Hz, 2H), 2.91 (t, J=11.7 Hz, 1H), 2.81 (t, J=11.2 Hz, 1H), 2.62-2.47 (m, 4H), 2.31 (d, J=1.4 Hz, 3H), 1.35 (d, J=6.2 Hz, 3H), 1.11 (td, J=7.2, 1.4 Hz, 3H).

### Example 245: (2R,6R)-4-(8-Cyano-quinoxalin-5-yl)-6-methyl-morpholine-2-carboxylic acid (1-methyl-pyrrolidin-2-ylmethyl)-amide

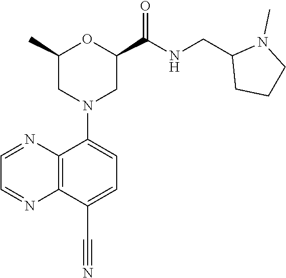 [SMILES: C[C@@H]1CN(c2ccc(C#N)c3nccnc23)C[C@H](C(=O)NCC2CCCN2C)O1]

The title compound was prepared from (2R,6R)-4-(8-Cyano-quinoxalin-5-yl)-6-methyl-morpholine-2-carboxylic acid and C-(1-Methyl-pyrrolidin-2-yl)-methylamine. MS:395 [M+H]⁺. ¹H NMR (400 MHz, Methanol-d₄) δ 8.97 (s, 1H), 8.93 (s, 1H), 8.14 (d, 1H), 7.26 (d, J=8.3 Hz, 1H), 4.58 (d, J=12.3 Hz, 1H), 4.44 (d, J=10.4 Hz, 1H), 4.20-4.04 (m, 2H), 3.58-3.47 (m, 1H), 3.24 (q, J=6.8, 6.4 Hz, 1H), 3.08 (dt, J=9.6, 4.9 Hz, 1H), 2.98-2.77 (m, 2H), 2.48 (s, 1H), 2.41 (s, 3H), 2.35-2.25 (m, 1H), 2.02-1.91 (m, 1H), 1.83-1.74 (m, 2H), 1.62 (dt, J=13.3, 7.4 Hz, 1H), 1.36 (d, J=6.1 Hz, 3H).

### Example 246: (2R,6R)-4-(8-Cyano-quinoxalin-5-yl)-6-methyl-morpholine-2-carboxylic acid (2-hydroxy-2-methyl-propyl)-amide

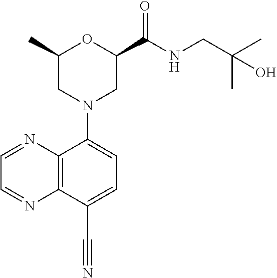 [SMILES: C[C@@H]1CN(c2ccc(C#N)c3nccnc23)C[C@H](C(=O)NCC(C)(C)O)O1]

The title compound was prepared from (2R,6R)-4-(8-Cyano-quinoxalin-5-yl)-6-methyl-morpholine-2-carboxylic acid and 1-amino-2-methylpropan-2-ol. MS: 370 [M+H]⁺. ¹H NMR (400 MHz, Methanol-d₄) δ 8.97 (s, 1H), 8.93 (s, 1H), 8.14 (d, J=8.3 Hz, 1H), 7.26 (d, J=8.3 Hz, 1H), 4.59 (d, J=12.2 Hz, 1H), 4.48 (dd, J=10.8, 2.7 Hz, 1H), 4.22-4.02 (m, 2H), 3.29 (s, 2H), 2.93 (t, J=11.5 Hz, 1H), 2.84 (t, J=11.2 Hz, 1H), 1.37 (d, J=6.1 Hz, 3H), 1.21 (s, 6H).

### Example 247: (2R,6R)-4-(8-Cyano-quinoxalin-5-yl)-6-methyl-morpholine-2-carboxylic acid [1-(2,2,2-trifluoro-ethyl)-piperidin-4-yl]-amide

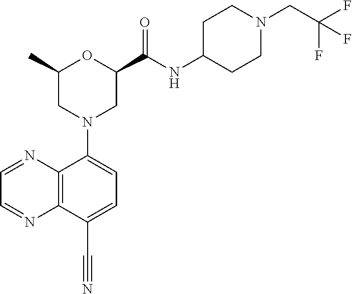 [SMILES: C[C@@H]1CN(c2ccc(C#N)c3nccnc23)C[C@H](C(=O)NC2CCN(CC(F)(F)F)CC2)O1]

The title compound was prepared from (2R,6R)-4-(8-Cyano-quinoxalin-5-yl)-6-methyl-morpholine-2-carboxylic acid and 1-(2,2,2-trifluoroethyl)piperidin-4-amine. MS: 463 [M+H]⁺. ¹H NMR (400 MHz, Methanol-d₄) δ 8.97 (s, 1H), 8.93 (s, 1H), 8.14 (d, J=8.3 Hz, 1H), 7.25 (d, J=8.3 Hz, 1H), 4.58-4.49 (m, 1H), 4.41 (dt, J=10.7, 1.9 Hz, 1H), 4.15 (d, J=12.4 Hz, 1H), 4.12-4.05 (m, 1H), 3.85-3.72 (m, 1H), 3.15-2.96 (m, 4H), 2.92 (t, J=11.4 Hz, 1H), 2.82 (t, J=11.2 Hz, 1H), 2.50 (t, J=11.7 Hz, 2H), 1.83 (d, J=12.3 Hz, 2H), 1.67 (q, J=12.0 Hz, 2H), 1.36 (d, J=6.1 Hz, 3H).

### Example 248: (2R,6R)-4-(8-Cyano-quinoxalin-5-yl)-6-methyl-morpholine-2-carboxylic acid ((R)-1-carbamoyl-propyl)-amide

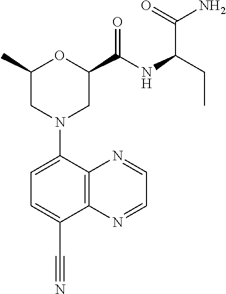 [SMILES: CC[C@@H](NC(=O)[C@H]1CN(c2ccc(C#N)c3nccnc23)C[C@@H](C)O1)C(N)=O]

The title compound was prepared from (2R,6R)-4-(8-Cyano-quinoxalin-5-yl)-6-methyl-morpholine-2-carboxylic acid and (r)-(−)-2-aminobutanamide hydrochloride. MS: 383 [M+H]⁺. H NMR (400 MHz, Methanol-d₄) δ 8.97 (s, 1H), 8.93 (s, 1H), 8.15 (dd, J=8.3, 1.6 Hz, 1H), 7.27 (dd, J=8.3, 1.6 Hz, 1H), 4.59 (dd, J=12.1, 2.4 Hz, 1H), 4.48 (dt, J=10.7, 2.2 Hz, 1H), 4.41 (t, J=6.4 Hz, 1H), 4.15 (td, J=11.9, 9.7, 4.1 Hz, 2H), 2.89 (dt, J=34.8, 11.5 Hz, 2H), 1.90 (dq, J=13.6, 7.7, 6.6 Hz, 1H), 1.81-1.66 (m, 1H), 1.37 (dd, J=6.1, 1.6 Hz, 3H), 0.97 (td, J=7.5, 1.6 Hz, 3H).

### Example 249: (2R,6R)-4-(8-Cyano-quinoxalin-5-yl)-6-methyl-morpholine-2-carboxylic acid ((S)-3,3,3-trifluoro-2-hydroxy-propyl)-amide

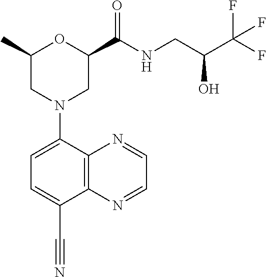 [SMILES: C[C@@H]1CN(c2ccc(C#N)c3nccnc23)C[C@H](C(=O)NC[C@H](O)C(F)(F)F)O1]

The title compound was prepared from (2R,6R)-4-(8-Cyano-quinoxalin-5-yl)-6-methyl-morpholine-2-carboxylic acid and (2S)-3-amino-1,1,1-trifluoropropan-2-olhydrochloride. MS: 410 [M+H]⁺. ¹H NMR (400 MHz, Methanol-d₄) δ 8.98 (s, 1H), 8.94 (s, 1H), 8.33-8.05 (m, 1H), 7.27 (d, J=8.3 Hz, 1H), 4.58 (d, J=12.3 Hz, 1H), 4.46 (dd, J=10.9, 2.6 Hz, 1H), 4.14 (dd, J=13.6, 9.7 Hz, 3H), 3.67 (dd, J=13.9, 4.1 Hz, 1H), 3.40 (dd, J=13.9, 8.2 Hz, 1H), 2.92 (t, J=11.5 Hz, 1H), 2.83 (t, J=11.2 Hz, 1H), 1.36 (d, J=6.1 Hz, 3H).

### Example 250: (2R,6R)-4-(8-Cyano-quinoxalin-5-yl)-6-methyl-morpholine-2-carboxylic acid ((R)-3,3,3-trifluoro-2-hydroxy-propyl)-amide

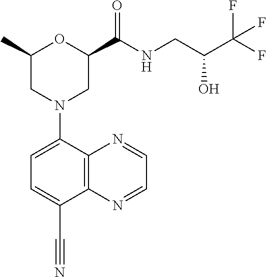 [SMILES: C[C@@H]1CN(c2ccc(C#N)c3nccnc23)C[C@H](C(=O)NC[C@@H](O)C(F)(F)F)O1]

The title compound was prepared from (2R,6R)-4-(8-Cyano-quinoxalin-5-yl)-6-methyl-morpholine-2-carboxylic acid and (2R)-3-amino-1,1,1-trifluoropropan-2-ol hydrochloride. MS: 410 [M+H]⁺. ¹H NMR (400 MHz, Methanol-d₄) δ 8.97 (s, 1H), 8.93 (s, 1H), 8.14 (dd, J=8.5, 1.6 Hz, 1H), 7.25 (d, J=8.5 Hz, 1H), 4.57 (dd, J=12.2, 2.4 Hz, 1H), 4.46 (dt, J=10.8, 2.1 Hz, 1H), 4.23-4.05 (m, 3H), 3.66 (dd, J=13.9, 4.1 Hz, 1H), 3.47-3.35 (m, 2H), 2.91 (t, J=11.5 Hz, 1H), 2.82 (t, J=11.2 Hz, 1H), 1.36 (d, J=6.1 Hz, 3H).

### Example 251: 8-[(2R,6R)-2-(2,2-Dioxo-2lambda6-thia-6-aza-spiro[3.3]heptane-6-carbonyl)-6-methyl-morpholin-4-yl]-quinoxaline-5-carbonitrile

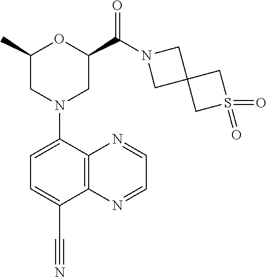 [SMILES: C[C@@H]1CN(c2ccc(C#N)c3nccnc23)C[C@H](C(=O)N2CC3(C2)CS(=O)(=O)C3)O1]

The title compound was prepared from (2R,6R)-4-(8-Cyano-quinoxalin-5-yl)-6-methyl-morpholine-2-carboxylic acid and 2-thia-6-azaspiro[3.3]heptane 2,2-dioxide hydrochloride. MS: 428 [M+H]⁺. ¹H NMR (400 MHz, Methanol-d₄) δ 8.97 (s, 1H), 8.92 (s, 1H), 8.15 (dd, J=8.6, 2.9 Hz, 1H), 7.25 (dd, J=8.4, 2.9 Hz, 1H), 4.75 (s, 2H), 4.59 (d, J=10.6 Hz, 1H), 4.50 (d, J=12.5 Hz, 1H), 4.42 (s, 4H), 4.29 (s, 2H), 4.11-3.98 (m, 2H), 3.07-2.97 (m, 1H), 2.82 (t, J=11.0 Hz, 1H), 1.33 (dd, J=6.7, 2.9 Hz, 3H).

### Example 252: (2R,6R)-4-(8-Cyano-quinoxalin-5-yl)-6-methyl-morpholine-2-carboxylic acid (2-hydroxy-3-methoxy-propyl)-amide

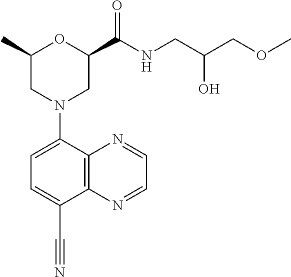

The title compound was prepared from (2R,6R)-4-(8-Cyano-quinoxalin-5-yl)-6-methyl-morpholine-2-carboxylic acid and 1-amino-3-methoxypropan-2-ol. MS: 386 [M+H]⁺. ¹H NMR (400 MHz, Methanol-d₄) δ 8.97 (s, 1H), 8.93 (s, 1H), 8.14 (d, J=8.2 Hz, 1H), 7.25 (d, J=8.3 Hz, 1H), 4.57 (d, J=12.3 Hz, 1H), 4.47-4.41 (m, 1H), 4.13 (dd, J=20.4, 8.5 Hz, 2H), 3.86 (q, J=5.0, 4.5 Hz, 1H), 3.53-3.36 (m, 6H), 3.31-3.22 (m, 1H), 2.92 (td, J=12.2, 11.6, 2.5 Hz, 1H), 2.83 (t, J=11.2 Hz, 1H), 1.36 (d, J=6.1 Hz, 3H).

### Example 253: (2R,6R)-4-(8-Cyano-quinoxalin-5-yl)-6-methyl-morpholine-2-carboxylic acid (4-fluoro-1-methyl-piperidin-4-ylmethyl)-amide

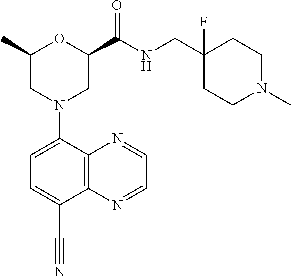

The title compound was prepared from (2R,6R)-4-(8-Cyano-quinoxalin-5-yl)-6-methyl-morpholine-2-carboxylic acid and 1-(4-fluoro-1-methylpiperidin-4-yl)methanamine. MS: 427 [M+H]⁺. ¹H NMR (400 MHz, Methanol-d₄) δ 8.98 (s, 1H), 8.93 (s, 1H), 8.15 (d, J=8.3 Hz, 1H), 7.26 (d, J=8.3 Hz, 1H), 4.58 (d, J=12.2 Hz, 1H), 4.48 (dd, J=10.8, 2.7 Hz, 1H), 4.23-4.03 (m, 2H), 3.58-3.41 (m, 2H), 2.94 (t, J=11.4 Hz, 1H), 2.84 (t, J=11.2 Hz, 1H), 2.71 (d, J=11.8 Hz, 2H), 2.32 (d, J=10.8 Hz, 5H), 1.93-1.64 (m, 4H), 1.37 (d, J=6.1 Hz, 3H).

### Example 254: (2R,6R)-4-(8-Cyano-quinoxalin-5-yl)-6-methyl-morpholine-2-carboxylic acid [1-(2,2,2-trifluoro-ethyl)-azetidin-3-yl]-amide

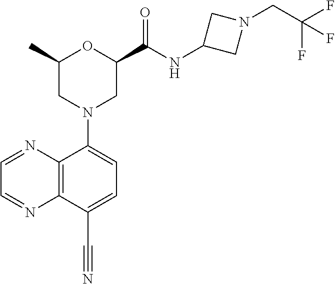 [SMILES: C[C@@H]1CN(c2ccc(C#N)c3nccnc23)C[C@H](C(=O)NC2CN(CC(F)(F)F)C2)O1]

The title compound was prepared from (2R,6R)-4-(8-Cyano-quinoxalin-5-yl)-6-methyl-morpholine-2-carboxylic acid and 1-(2,2,2-trifluoroethyl)azetidin-3-amine. MS: 435 [M+H]⁺. ¹H NMR (400 MHz, Methanol-d₄) δ 9.01-8.95 (m, 1H), 8.95-8.88 (m, 1H), 8.14 (d, J=8.2 Hz, 1H), 7.25 (d, J=8.3 Hz, 1H), 4.62-4.49 (m, 2H), 4.43 (dd, J=10.8, 2.8 Hz, 1H), 4.22-4.05 (m, 2H), 3.81 (q, J=6.5 Hz, 2H), 3.43-3.31 (m, 2H), 3.19 (q, J=9.6 Hz, 2H), 2.91 (t, J=11.5 Hz, 1H), 2.83 (dd, J=12.1, 10.2 Hz, 1H), 1.38 (d, J=6.2 Hz, 3H).

### Example 255: (2R,6R)-4-(8-cyanoquinoxalin-5-yl)-6-methyl-N-[2-(methylsulfamoyl)ethyl]morpholine-2-carboxamide

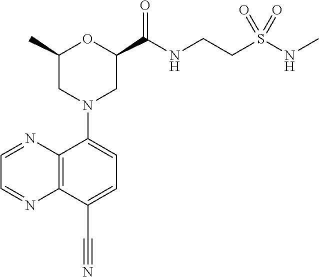 [SMILES: CNS(=O)(=O)CCNC(=O)[C@H]1CN(c2ccc(C#N)c3nccnc23)C[C@@H](C)O1]

The title compound was prepared from (2R,6R)-4-(8-Cyano-quinoxalin-5-yl)-6-methyl-morpholine-2-carboxylic acid and 2-amino-n-methylethanesulfonamide hydrochloride. MS: 419 [M+H]⁺. ¹H NMR (400 MHz, DMSO-d₆) δ 9.07 (d, J=1.8 Hz, 1H), 8.99 (d, J=1.8 Hz, 1H), 8.25 (d, J=8.4 Hz, 1H), 7.99 (t, J=5.9 Hz, 1H), 7.26 (d, J=8.4 Hz, 1H), 7.03 (s, 1H), 4.59-4.44 (m, 1H), 4.30 (dd, J=10.8, 2.8 Hz, 1H), 4.09 (d, J=12.4 Hz, 1H), 3.98 (ddd, J=10.4, 6.2, 2.3 Hz, 1H), 3.48 (td, J=8.4, 7.7, 4.0 Hz, 2H), 3.18 (dd, J=7.6, 6.6 Hz, 2H), 2.90 (dd, J=12.4, 10.8 Hz, 1H), 2.79 (dd, J=12.5, 10.4 Hz, 1H), 2.59 (s, 3H), 1.26 (d, J=6.2 Hz, 3H).

### Example 256: (2R,6R)-4-(8-cyanoquinoxalin-5-yl)-N-(2-methanesulfonylethyl)-6-methyl morpholine-2-carboxamide

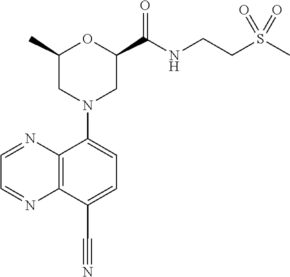 [SMILES: C[C@@H]1CN(c2ccc(C#N)c3nccnc23)C[C@H](C(=O)NCCS(C)(=O)=O)O1]

The title compound was prepared from (2R,6R)-4-(8-Cyano-quinoxalin-5-yl)-6-methyl-morpholine-2-carboxylic acid and 2-methanesulfonylethan-1-amine. MS: 404 [M+H]⁺. ¹H NMR (400 MHz, DMSO-d₆) δ 9.07 (d, J=1.8 Hz, 1H), 8.99 (d, J=1.8 Hz, 1H), 8.25 (d, J=8.4 Hz, 1H), 8.05 (t, J=5.9 Hz, 1H), 7.26 (d, J=8.4 Hz, 1H), 4.55-4.44 (m, 1H), 4.30 (dd, J=10.8, 2.7 Hz, 1H), 4.09 (d, J=12.3 Hz, 1H), 3.97 (ddd, J=10.4, 6.2, 2.3 Hz, 1H), 3.56 (q, J=6.6 Hz, 2H), 3.02 (s, 3H), 3.32-3.27 (m, 2H), 2.90 (dd, J=12.5, 10.8 Hz, 1H), 2.79 (dd, J=12.5, 10.4 Hz, 1H), 1.26 (d, J=6.3 Hz, 3H).

### Example 257: (2R,6R)-4-(8-cyanoquinoxalin-5-yl)-N-[(1,1-dioxo-1λ⁶-thiolan-3-yl)methyl]-6-methylmorpholine-2-carboxamide

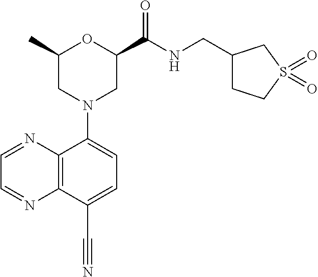 [SMILES: C[C@@H]1CN(c2ccc(C#N)c3nccnc23)C[C@H](C(=O)NCC2CCS(=O)(=O)C2)O1]

The title compound was prepared from (2R,6R)-4-(8-Cyano-quinoxalin-5-yl)-6-methyl-morpholine-2-carboxylic acid and 3-(aminomethyl)-1λ⁶-thiolane-1,1-dione. MS: 430 [M+H]⁺. H NMR (400 MHz, DMSO-d₆) δ 9.07 (d, J=1.8 Hz, 1H), 8.99 (d, J=1.8 Hz, 1H), 8.25 (d, J=8.4 Hz, 1H), 8.06 (t, J=6.2 Hz, 1H), 7.27 (dd, J=8.5, 1.2 Hz, 1H), 4.48 (dd, J=12.3, 2.4 Hz, 1H), 4.30 (dd, J=10.8, 2.7 Hz, 1H), 4.15-4.05 (m, 1H), 3.97 (ddd, J=10.4, 6.2, 2.4 Hz, 1H), 3.27-3.22 (m, 2H), 3.18 (ddd, J=12.6, 8.1, 3.8 Hz, 2H), 3.04 (dt, J=13.2, 8.7 Hz, 1H), 2.93 (ddd, J=12.5, 10.8, 1.9 Hz, 1H), 2.81 (dd, J=12.9, 9.8 Hz, 2H), 2.58 (dq, J=14.0, 7.1, 6.4 Hz, 1H), 2.22-2.08 (m, 1H), 1.78 (dq, J=13.3, 9.2 Hz, 1H), 1.27 (d, J=6.2 Hz, 3H).

### Example 258: (2R,6R)-4-(8-cyanoquinoxalin-5-yl)-N-[(1,1-dioxo-1λ⁶-thietan-3-yl)methyl]-6-methylmorpholine-2-carboxamide

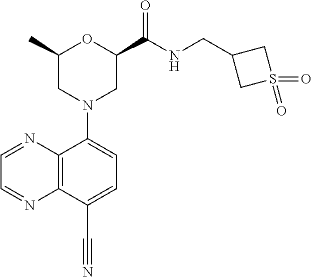 [SMILES: C[C@@H]1CN(c2ccc(C#N)c3nccnc23)C[C@H](C(=O)NCC2CS(=O)(=O)C2)O1]

The title compound was prepared from (2R,6R)-4-(8-Cyano-quinoxalin-5-yl)-6-methyl-morpholine-2-carboxylic acid and 3-(aminomethyl)-1λ⁶-thietane-1,1-dione. MS: 416 [M+H]⁺. ¹H NMR (400 MHz, DMSO-d₆) δ 9.07 (d, J=1.8 Hz, 1H), 8.99 (d, J=1.8 Hz, 1H), 8.25 (d, J=8.4 Hz, 1H), 8.22 (t, J=6.6 Hz, 1H), 7.27 (d, J=8.4 Hz, 1H), 4.49 (d, J=12.3 Hz, 1H), 4.29 (dd, J=10.7, 2.7 Hz, 1H), 4.25-4.17 (m, 2H), 4.10 (d, J=12.3 Hz, 1H), 4.0-3.88 (m, 1H), 3.41-3.35 (m, 1H), 2.90 (dd, J=12.4, 10.8 Hz, 1H), 2.84-2.75 (m, 1H), 2.73-2.65 (m, 1H), 1.27 (d, J=6.2 Hz, 3H).

### Example 259: (2R,6R)-4-(8-cyanoquinoxalin-5-yl)-N-[2-(1,1-dioxo-1λ⁶-thietan-3-yl)ethyl]-6-methylmorpholine-2-carboxamide

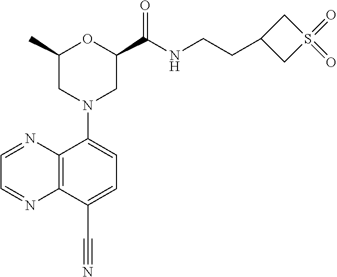 [SMILES: C[C@@H]1CN(c2ccc(C#N)c3nccnc23)C[C@H](C(=O)NCCC2CS(=O)(=O)C2)O1]

The title compound was prepared from (2R,6R)-4-(8-Cyano-quinoxalin-5-yl)-6-methyl-morpholine-2-carboxylic acid and 3-(2-aminoethyl)-1λ⁶-thietane-1,1-dione. MS: 430 [M+H]⁺. H NMR (400 MHz, DMSO-d₆) δ 9.07 (d, J=1.8 Hz, 1H), 8.99 (d, J=1.8 Hz, 1H), 8.25 (d, J=8.4 Hz, 1H), 7.90 (t, J=6.1 Hz, 1H), 7.27 (d, J=8.5 Hz, 1H), 4.99 (s, 1H), 4.49 (d, J=12.4 Hz, 1H), 4.27 (dd, J=10.8, 2.7 Hz, 1H), 4.25-4.17 (m, 2H), 4.09 (d, J=12.4 Hz, 1H), 4.01-3.92 (m, 1H), 3.86-3.79 (m, 2H), 3.12 (q, J=6.5 Hz, 2H), 2.90 (dd, J=12.4, 10.8 Hz, 1H), 2.80 (dd, J=12.4, 10.3 Hz, 1H), 2.48-2.41 (m, 1H), 1.78 (q, J=6.9 Hz, 2H), 1.27 (d, J=6.2 Hz, 3H).

### Example 260: (2R,6R)-4-(8-cyanoquinoxalin-5-yl)-N-(3-methanesulfonylpropyl)-6-methylmorpholine-2-carboxamide

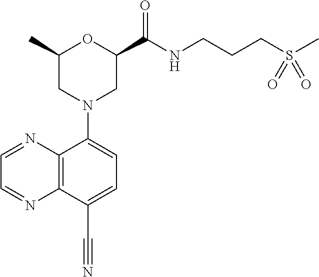 [SMILES: C[C@@H]1CN(c2ccc(C#N)c3nccnc23)C[C@H](C(=O)NCCCS(C)(=O)=O)O1]

The title compound was prepared from (2R,6R)-4-(8-Cyano-quinoxalin-5-yl)-6-methyl-morpholine-2-carboxylic acid and 3-methanesulfonylpropan-1-amine. MS: 418 [M+H]⁺. ¹H NMR (400 MHz, DMSO-d₆) δ 9.07 (d, J=1.8 Hz, 1H), 8.99 (d, J=1.8 Hz, 1H), 8.25 (d, J=8.4 Hz, 1H), 7.98 (t, J=6.1 Hz, 1H), 7.27 (d, J=8.4 Hz, 1H), 4.50 (d, J=12.4 Hz, 1H), 4.29 (dd, J=10.8, 2.7 Hz, 1H), 4.10 (d, J=12.3 Hz, 1H), 3.97 (ddd, J=10.3, 6.2, 2.3 Hz, 1H), 3.27-3.19 (m, 2H), 3.13-3.05 (m, 2H), 2.92 (dd, J=12.4, 10.8 Hz, 1H), 2.81 (dd, J=12.4, 10.4 Hz, 1H), 1.86 (dt, J=14.8, 7.0 Hz, 2H), 1.27 (d, J=6.2 Hz, 3H).

### Example 261: (2R,6R)-4-(8-cyanoquinoxalin-5-yl)-N-[(2S)-3-(dimethylamino)-2-hydroxypropyl]-6-methylmorpholine-2-carboxamide & Example 262: (2R,6R)-4-(8-cyanoquinoxalin-5-yl)-N-[(2R)-3-(dimethylamino)-2-hydroxypropyl]-6-methylmorpholine-2-carboxamide

The title compounds were made from (2R,6R)-4-(8-Cyano-quinoxalin-5-yl)-6-methyl-morpholine-2-carboxylic acid and 1-amino-3-(dimethylamino)propan-2-ol, and were separated by SFC. The conditions were: column, IG-H, Prep SFC-P100; mobile phase, methanol+20 mM NH₄OH, 45° C./80 bar, 100 g/min; detector, PDA. The configurations of the structures were tentatively assigned.

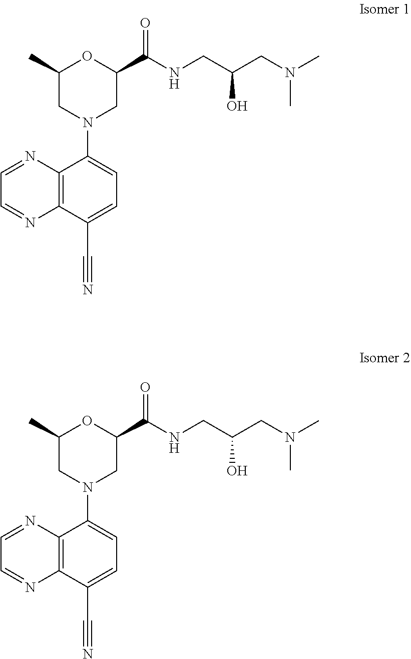 [SMILES: C[C@@H]1CN(c2ccc(C#N)c3nccnc23)C[C@H](C(=O)NC[C@H](O)CN(C)C)O1 ; C[C@@H]1CN(c2ccc(C#N)c3nccnc23)C[C@H](C(=O)NC[C@@H](O)CN(C)C)O1]

Isomer 1: MS:399 [M+H]⁺. ¹H NMR (400 MHz, DMSO-d₆) δ 9.08 (s, 1H), 9.0 (s, 1H), 8.26 (d, J=8.4 Hz, 1H), 7.81 (d, J=7.0 Hz, 1H), 7.27 (d, J=8.4 Hz, 1H), 4.78 (d, J=4.0 Hz, 1H), 4.52 (d, J=12.3 Hz, 1H), 4.30 (d, J=10.6 Hz, 1H), 4.11 (d, J=12.5 Hz, 1H), 3.98 (d, J=8.7 Hz, 1H), 3.74-3.61 (m, 1H), 3.30-3.23 (m, 3H), 3.10 (dt, J=13.6, 6.7 Hz, 1H), 3.01-2.76 (m, 2H), 2.23 (t, J=7.4 Hz, 1H), 2.17 (d, J=2.3 Hz, 6H), 1.27 (d, J=6.4 Hz, 3H).

Isomer 2: MS:399 [M+H]⁺. ¹H NMR (400 MHz, DMSO-d₆) δ 9.08 (s, 1H), 9.0 (s, 1H), 8.26 (d, J=8.5 Hz, 1H), 7.81 (s, 1H), 7.27 (d, J=8.4 Hz, 1H), 4.78 (s, 1H), 4.52 (d, J=12.4 Hz, 1H), 4.30 (d, J=10.8 Hz, 1H), 4.11 (d, J=12.5 Hz, 1H), 3.98 (d, J=9.2 Hz, 1H), 3.65 (s, 1H), 3.28-3.19 (m, 0H), 3.19-3.07 (m, 1H), 2.88 (ddd, J=41.7, 23.5, 12.0 Hz, 2H), 2.26-2.20 (m, 1H), 2.17 (d, J=2.2 Hz, 6H), 1.27 (d, J=6.2 Hz, 3H).

### Example 263: (2R,6R)-4-(8-cyanoquinoxalin-5-yl)-6-methyl-N-{[(3S)-4-methylmorpholin-3-yl]methyl}morpholine-2-carboxamide & Example 264: (2R,6R)-4-(8-cyanoquinoxalin-5-yl)-6-methyl-N-{[(3R)-4-methylmorpholin-3-yl]methyl}morpholine-2-carboxamide

The title compounds were made from (2R,6R)-4-(8-Cyano-quinoxalin-5-yl)-6-methyl-morpholine-2-carboxylic acid and 1-(4-methylmorpholin-3-yl)methanamine, and were separated by SFC. The conditions were: column, ADH, Prep SFC-P100; mobile phase, methanol+20 mM NH₄OH, 45° C./80 bar, 100 g/min; detector, PDA. The configurations of the structures were tentatively assigned.

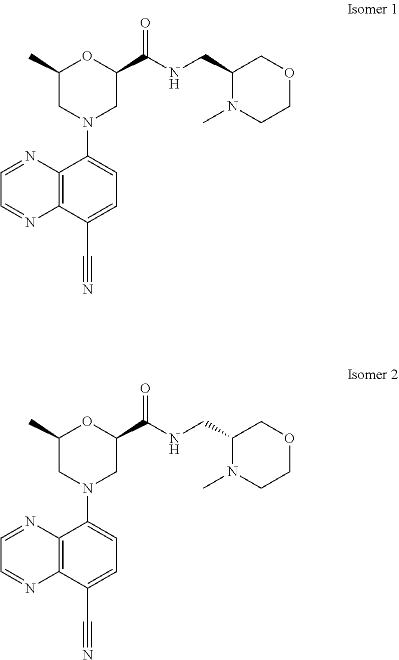 [SMILES: C[C@@H]1CN(c2ccc(C#N)c3nccnc23)C[C@H](C(=O)NC[C@H]2COCCN2C)O1 ; C[C@@H]1CN(c2ccc(C#N)c3nccnc23)C[C@H](C(=O)NC[C@@H]2COCCN2C)O1]

Isomer 1: MS:411 [M+H]⁺. ¹H NMR (400 MHz, DMSO-d₆) δ 9.08 (d, J=1.8 Hz, 1H), 9.0 (d, J=1.8 Hz, 1H), 8.25 (d, J=8.4 Hz, 1H), 7.72 (t, J=6.0 Hz, 1H), 7.27 (d, J=8.5 Hz, 1H), 4.50 (dt, J=12.4, 2.3 Hz, 1H), 4.30 (dd, J=10.7, 2.7 Hz, 1H), 4.13-4.07 (m, 1H), 3.98 (ddd, J=10.4, 6.3, 2.4 Hz, 1H), 3.65 (ddd, J=11.3, 8.4, 3.3 Hz, 3H), 3.49-3.32 (m, 2H), 3.15 (dd, J=11.3, 9.5 Hz, 1H), 3.12-3.01 (m, 1H), 2.99-2.87 (m, 1H), 2.86-2.77 (m, 1H), 2.70-2.60 (m, 2H), 2.25 (s, 3H), 2.20-2.09 (m, 2H), 1.27 (d, J=6.2 Hz, 3H).

Isomer 2: MS:411 [M+H]⁺. ¹H NMR (400 MHz, DMSO-d₆) δ 9.08 (d, J=1.8 Hz, 1H), 9.0 (d, J=1.8 Hz, 1H), 8.25 (d, J=8.4 Hz, 1H), 7.71 (t, J=6.0 Hz, 1H), 7.27 (d, J=8.4 Hz, 1H), 4.53-4.46 (m, 1H), 4.30 (dd, J=10.7, 2.7 Hz, 1H), 4.14-4.07 (m, 1H), 3.98 (ddd, J=10.4, 6.2, 2.3 Hz, 1H), 3.70-3.61 (m, 2H), 3.44 (td, J=10.9, 2.5 Hz, 1H), 3.36 (ddd, J=13.7, 6.2, 3.2 Hz, 1H), 3.15 (dd, J=11.4, 9.5 Hz, 1H), 3.06 (dt, J=13.3, 6.4 Hz, 1H), 2.91 (dd, J=12.4, 10.8 Hz, 1H), 2.82 (dd, J=12.5, 10.4 Hz, 1H), 2.72-2.60 (m, 2H), 2.25 (s, 3H), 2.19-2.09 (m, 2H), 1.27 (d, J=6.2 Hz, 3H).

### Example 265: (2R,6R)-4-(8-cyanoquinoxalin-5-yl)-6-methyl-N-[2-(morpholin-4-yl)ethyl]morpholine-2-carboxamide

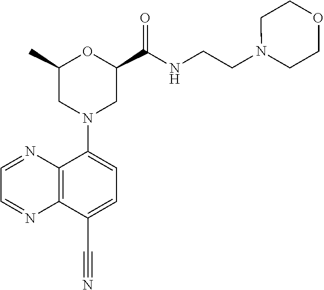 [SMILES: C[C@@H]1CN(c2ccc(C#N)c3nccnc23)C[C@H](C(=O)NCCN2CCOCC2)O1]

The title compound was prepared from (2R,6R)-4-(8-Cyano-quinoxalin-5-yl)-6-methyl-morpholine-2-carboxylic acid and 2-(morpholin-4-yl)ethan-1-amine. MS:411 [M+H]⁺. ¹H NMR (400 MHz, DMSO-d₆) δ 9.08 (d, J=1.8 Hz, 1H), 9.0 (d, J=1.8 Hz, 1H), 8.25 (d, J=8.4 Hz, 1H), 7.72 (t, J=5.8 Hz, 1H), 7.27 (d, J=8.5 Hz, 1H), 4.50 (dt, J=12.3, 2.4 Hz, 1H), 4.28 (dd, J=10.8, 2.7 Hz, 1H), 4.11 (dt, J=12.5, 2.2 Hz, 1H), 3.98 (ddd, J=10.4, 6.3, 2.4 Hz, 1H), 3.57 (t, J=4.7 Hz, 5H), 3.27-3.18 (m, 1H), 2.91 (dd, J=12.5, 10.8 Hz, 1H), 2.81 (dd, J=12.5, 10.4 Hz, 1H), 2.42-2.35 (m, 7H), 1.27 (d, J=6.2 Hz, 3H).

### Example 266: (2R,6R)-4-(8-Cyano-quinazolin-5-yl)-6-methyl-morpholine-2-carboxylic acid [2-(ethyl-methyl-amino)-ethyl]-amide

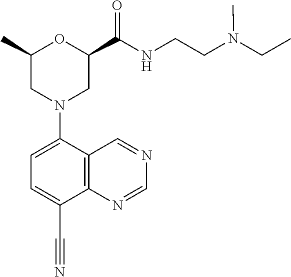 [SMILES: CCN(C)CCNC(=O)[C@H]1CN(c2ccc(C#N)c3ncncc23)C[C@@H](C)O1]

The title compound was prepared from 5-((2R,6R)-2-Hydroxymethyl-6-methyl-morpholin-4-yl)-quinazoline-8-carbonitrile and (2-aminoethyl)(ethyl)methylamine. MS: 383 [M+H]⁺. ¹H NMR (400 MHz, Methanol-d₄) δ 9.74 (s, 1H), 9.35 (s, 1H), 8.34 (d, J=8.2 Hz, 1H), 7.34 (d, J=8.2 Hz, 1H), 4.51 (dd, J=10.9, 2.6 Hz, 1H), 4.19 (ddt, J=9.5, 6.4, 3.6 Hz, 1H), 3.87 (dd, J=12.3, 2.4 Hz, 1H), 3.65-3.54 (m, 1H), 3.42 (hept, J=6.8 Hz, 2H), 2.97 (t, J=11.5 Hz, 1H), 2.90-2.82 (m, 1H), 2.54 (dt, J=20.7, 6.9 Hz, 4H), 2.30 (s, 3H), 1.36 (d, J=6.2 Hz, 3H), 1.11 (t, J=7.2 Hz, 3H).

### Example 267: (2R,6R)-4-(8-Cyano-quinazolin-5-yl)-6-methyl-morpholine-2-carboxylic acid ((S)-4-methyl-morpholin-2-ylmethyl)-amide

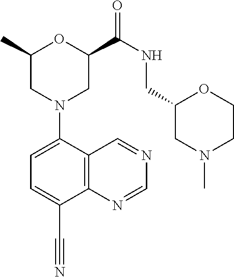 [SMILES: C[C@@H]1CN(c2ccc(C#N)c3ncncc23)C[C@H](C(=O)NC[C@H]2CN(C)CCO2)O1]

The title compound was prepared from 5-((2R,6R)-2-Hydroxymethyl-6-methyl-morpholin-4-yl)-quinazoline-8-carbonitrile and (s)-4-methyl-2-(aminomethyl)morpholine. MS: 411 [M+H]⁺. ¹HNMR (400 MHz, Methanol-d₄) δ 9.73 (s, 1H), 9.33 (s, 1H), 8.32 (d, J=8.1 Hz, 1H), 7.33 (d, J=8.2 Hz, 1H), 4.53 (dd, J=11.0, 2.5 Hz, 1H), 4.19 (dd, J=9.9, 6.2 Hz, 1H), 3.89 (t, J=12.7 Hz, 2H), 3.69-3.55 (m, 3H), 3.35 (qd, J=13.9, 5.7 Hz, 2H), 2.98 (t, J=11.5 Hz, 1H), 2.87 (t, J=11.3 Hz, 1H), 2.78 (d, J=11.5 Hz, 1H), 2.70 (d, J=11.7 Hz, 1H), 2.30 (s, 3H), 2.15 (td, J=11.7, 3.4 Hz, 1H), 1.88 (t, J=10.9 Hz, 1H), 1.37 (d, J=6.2 Hz, 3H).

### Example 268: (2R,6R)-4-(8-Cyano-quinazolin-5-yl)-6-methyl-morpholine-2-carboxylic acid (4-morpholin-4-yl-cyclohexyl)-amide

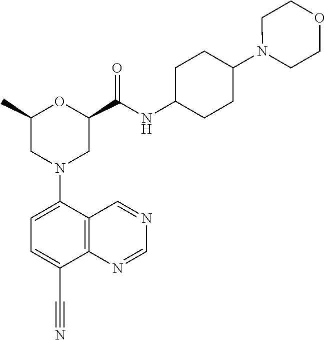 [SMILES: C[C@@H]1CN(c2ccc(C#N)c3ncncc23)C[C@H](C(=O)NC2CCC(N3CCOCC3)CC2)O1]

The title compound was prepared from 5-((2R,6R)-2-Hydroxymethyl-6-methyl-morpholin-4-yl)-quinazoline-8-carbonitrile and 4-Morpholin-4-yl-cyclohexylamine trifluoroacetate. MS: 465 [M+H]⁺. ¹H NMR (400 MHz, Methanol-d₄) δ 9.74 (s, 1H), 9.35 (s, 1H), 8.34 (dd, J=8.7, 3.1 Hz, 1H), 7.34 (dd, J=7.5, 2.8 Hz, 1H), 4.48 (d, J=10.4 Hz, 1H), 4.17 (s, 1H), 3.85 (d, J=12.4 Hz, 1H), 3.71 (q, J=4.3 Hz, 5H), 3.58 (d, J=12.5 Hz, 1H), 3.04-2.92 (m, 1H), 2.86 (t, J=10.7 Hz, 1H), 2.61 (q, J=4.1 Hz, 4H), 2.28 (s, 1H), 2.10-1.77 (m, 4H), 1.53-1.12 (m, 7H).

### Example 269: (2R,6R)-4-(8-Cyano-[1,7]naphthyridin-5-yl)-6-methyl-morpholine-2-carboxylic acid (3,3,3-trifluoro-2-hydroxy-propyl)-amide

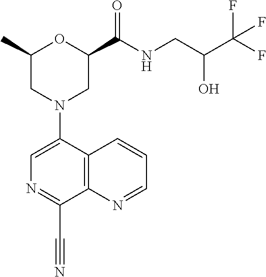 [SMILES: C[C@@H]1CN(c2cnc(C#N)c3ncccc23)C[C@H](C(=O)NCC(O)C(F)(F)F)O1]

The title compound was prepared from 5-[(2R,6R)-2-(hydroxymethyl)-6-methylmorpholin-4-yl]-1,7-naphthyridine-8-carbonitrile and 3-amino-1,1,1-trifluoropropan-2-ol. MS: 410 [M+H]⁺. ¹H NMR (400 MHz, Methanol-d₄) δ 9.14 (d, J=4.1 Hz, 1H), 8.66 (d, J=8.7 Hz, 1H), 8.37 (s, 1H), 7.85 (dd, J=8.8, 4.1 Hz, 1H), 4.54 (d, J=10.5 Hz, 1H), 4.16 (h, J=6.4, 6.0 Hz, 2H), 3.84 (d, J=12.2 Hz, 1H), 3.66 (dd, J=13.9, 4.0 Hz, 1H), 3.53 (d, J=12.1 Hz, 1H), 3.40 (dd, J=13.9, 8.2 Hz, 1H), 3.0-2.81 (m, 2H), 1.37 (d, J=6.2 Hz, 3H).

### Example 270: (2R,6R)-4-(8-Cyano-[1,7]naphthyridin-5-yl)-6-methyl-morpholine-2-carboxylic acid [2-(ethyl-methyl-amino)-ethyl]-amide

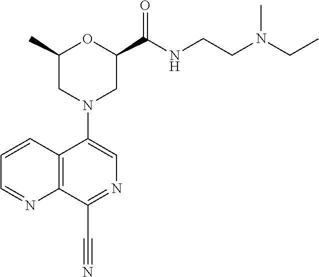 [SMILES: CCN(C)CCNC(=O)[C@H]1CN(c2cnc(C#N)c3ncccc23)C[C@@H](C)O1]

The title compound was prepared from 5-[(2R,6R)-2-(hydroxymethyl)-6-methylmorpholin-4-yl]-1,7-naphthyridine-8-carbonitrile and (2-aminoethyl)(ethyl)methylamine. MS: 383 [M+H]⁺. ¹H NMR (400 MHz, Methanol-d₄) δ 9.14 (d, J=4.2 Hz, 1H), 8.67 (d, J=8.7 Hz, 1H), 8.38 (s, 1H), 7.86 (dd, J=8.8, 4.2 Hz, 1H), 4.56-4.46 (m, 1H), 4.24-4.09 (m, 1H), 3.84 (d, J=12.0 Hz, 1H), 3.53 (d, J=12.1 Hz, 1H), 3.42 (hept, J=7.0 Hz, 2H), 2.91 (dt, J=22.8, 11.3 Hz, 2H), 2.54 (dt, J=20.8, 7.0 Hz, 4H), 2.30 (s, 3H), 1.37 (d, J=6.2 Hz, 3H), 1.11 (t, J=7.2 Hz, 3H).

### Example 271: (2R,6R)-4-(8-Cyano-[1,7]naphthyridin-5-yl)-6-methyl-morpholine-2-carboxylic acid (4-methyl-morpholin-2-ylmethyl)-amide

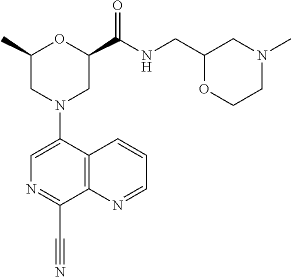

The title compound was prepared from 5-[(2R,6R)-2-(hydroxymethyl)-6-methylmorpholin-4-yl]-1,7-naphthyridine-8-carbonitrile and (4-methylmorpholin-2-yl)methanamine. MS: 411 [M+H]⁺. ¹H NMR (400 MHz, Methanol-d₄) δ 9.15 (d, J=4.1 Hz, 1H), 8.68 (d, J=8.6 Hz, 1H), 8.39 (s, 1H), 7.86 (dd, J=8.7, 4.2 Hz, 1H), 4.59-4.45 (m, 1H), 4.21-4.11 (m, 1H), 3.96-3.88 (m, 1H), 3.84 (d, J=12.1 Hz, 1H), 3.70-3.60 (m, 2H), 3.53 (d, J=12.1 Hz, 1H), 3.40 (ddd, J=13.9, 9.1, 4.8 Hz, 1H), 3.31-3.24 (m, 1H), 2.92 (dt, J=17.6, 11.8 Hz, 2H), 2.78 (d, J=11.5 Hz, 1H), 2.70 (d, J=11.7 Hz, 1H), 2.30 (s, 3H), 2.15 (td, J=11.7, 3.5 Hz, 1H), 1.91-1.83 (m, 1H), 1.37 (d, J=6.2 Hz, 3H).

### Example 272: (2R,6R)-4-(8-Cyano-[1,7]naphthyridin-5-yl)-6-methyl-morpholine-2-carboxylic acid (1-cyclopropylmethyl-pyrrolidin-3-yl)-amide

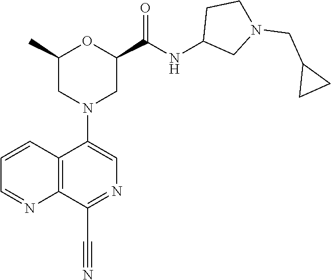 [SMILES: C[C@@H]1CN(c2cnc(C#N)c3ncccc23)C[C@H](C(=O)NC2CCN(CC3CC3)C2)O1]

The title compound was prepared from 5-[(2R,6R)-2-(hydroxymethyl)-6-methylmorpholin-4-yl]-1,7-naphthyridine-8-carbonitrile and 1-(cyclopropylmethyl)pyrrolidin-3-amine. MS: 421 [M+H]⁺. ¹H NMR (400 MHz, Methanol-d₄) δ 9.15 (d, J=4.0 Hz, 1H), 8.67 (dd, J=8.6, 1.6 Hz, 1H), 8.39 (s, 1H), 7.86 (dd, J=8.7, 4.1 Hz, 1H), 4.49 (dd, J=10.7, 2.8 Hz, 2H), 4.21-4.10 (m, 1H), 3.83 (d, J=12.1 Hz, 1H), 3.53 (d, J=12.2 Hz, 1H), 3.10-2.85 (m, 3H), 2.80-2.65 (m, 2H), 2.46 (td, J=9.8, 5.0 Hz, 1H), 2.37 (t, J=8.9 Hz, 3H), 1.74 (ddd, J=13.3, 10.6, 6.3 Hz, 1H), 1.38 (d, J=6.2 Hz, 3H), 0.94 (d, J=7.0 Hz, 1H), 0.68-0.48 (m, 2H), 0.26-0.11 (m, 2H).

### Example 273: (2R,6R)-4-(8-Cyano-[1,7]naphthyridin-5-yl)-6-methyl-morpholine-2-carboxylic acid (1-methyl-pyrrolidin-2-ylmethyl)-amide

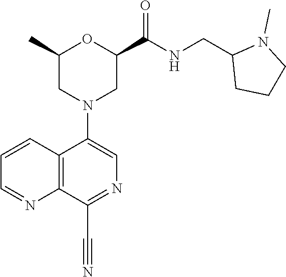 [SMILES: C[C@@H]1CN(c2cnc(C#N)c3ncccc23)C[C@H](C(=O)NCC2CCCN2C)O1]

The title compound was prepared from 5-[(2R,6R)-2-(hydroxymethyl)-6-methylmorpholin-4-yl]-1,7-naphthyridine-8-carbonitrile and C-(1-Methyl-pyrrolidin-2-yl)-methylamine. MS: 395 [M+H]⁺. ¹H NMR (400 MHz, Methanol-d₄) δ 9.15 (d, J=4.1 Hz, 1H), 8.75-8.63 (m, 1H), 8.39 (s, 1H), 7.86 (dd, J=8.7, 4.1 Hz, 1H), 4.52 (dd, J=10.9, 2.5 Hz, 1H), 4.22-4.03 (m, 1H), 3.85 (d, J=12.2 Hz, 1H), 3.52 (td, J=10.0, 4.4 Hz, 2H), 3.23 (ddd, J=13.6, 9.9, 6.4 Hz, 1H), 3.08 (dt, J=9.6, 4.7 Hz, 1H), 2.93 (ddt, J=21.2, 14.8, 7.9 Hz, 2H), 2.47 (s, 1H), 2.41 (s, 3H), 2.32-2.19 (m, 1H), 2.02-1.89 (m, 1H), 1.77 (qd, J=8.7, 8.3, 5.3 Hz, 2H), 1.61 (dq, J=14.2, 7.3 Hz, 1H), 1.37 (d, J=6.2 Hz, 3H).

### Example 274: (2R,6R)-4-(8-Cyano-[1,7]naphthyridin-5-yl)-6-methyl-morpholine-2-carboxylic acid ((S)-4-methyl-morpholin-2-ylmethyl)-amide

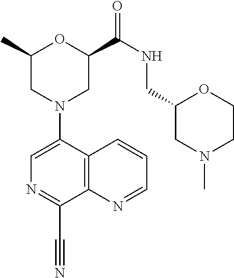 [SMILES: C[C@@H]1CN(c2cnc(C#N)c3ncccc23)C[C@H](C(=O)NC[C@H]2CN(C)CCO2)O1]

The title compound was prepared from 5-[(2R,6R)-2-(hydroxymethyl)-6-methylmorpholin-4-yl]-1,7-naphthyridine-8-carbonitrile and C—((S)-4-Methyl-morpholin-2-yl)-methylamine. MS: 411 [M+H]⁺. ¹H NMR (400 MHz, Methanol-d₄) δ 9.15 (d, J=4.1 Hz, 1H), 8.68 (d, J=8.6 Hz, 1H), 8.39 (s, 1H), 7.86 (dd, J=8.7, 4.2 Hz, 1H), 4.59-4.45 (m, 1H), 4.21-4.11 (m, 1H), 3.96-3.88 (m, 1H), 3.84 (d, J=12.1 Hz, 1H), 3.70-3.60 (m, 2H), 3.53 (d, J=12.1 Hz, 1H), 3.40 (ddd, J=13.9, 9.1, 4.8 Hz, 1H), 3.31-3.24 (m, 1H), 2.92 (dt, J=17.6, 11.8 Hz, 2H), 2.78 (d, J=11.5 Hz, 1H), 2.70 (d, J=11.7 Hz, 1H), 2.30 (s, 3H), 2.15 (td, J=11.7, 3.5 Hz, 1H), 1.91-1.83 (m, 1H), 1.37 (d, J=6.2 Hz, 3H).

### Example 275: (2R,6R)-4-(8-Cyano-[1,7]naphthyridin-5-yl)-6-methyl-morpholine-2-carboxylic acid ((R)-4-methyl-morpholin-2-ylmethyl)-amide

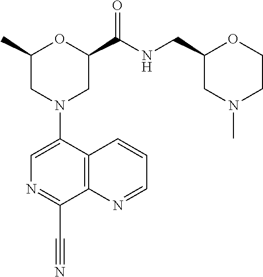 [SMILES: C[C@@H]1CN(c2cnc(C#N)c3ncccc23)C[C@H](C(=O)NC[C@@H]2CN(C)CCO2)O1]

The title compound was prepared from 5-[(2R,6R)-2-(hydroxymethyl)-6-methylmorpholin-4-yl]-1,7-naphthyridine-8-carbonitrile and C—((R)-4-Methyl-morpholin-2-yl)-methylamine. MS: 411 [M+H]⁺. ¹H NMR (400 MHz, Methanol-d₄) δ 9.15 (d, J=4.1 Hz, 1H), 8.68 (d, J=8.6 Hz, 1H), 8.39 (s, 1H), 7.86 (dd, J=8.7, 4.2 Hz, 1H), 4.59-4.45 (m, 1H), 4.21-4.11 (m, 1H), 3.96-3.88 (m, 1H), 3.84 (d, J=12.1 Hz, 1H), 3.70-3.60 (m, 2H), 3.53 (d, J=12.1 Hz, 1H), 3.40 (ddd, J=13.9, 9.1, 4.8 Hz, 1H), 3.31-3.24 (m, 1H), 2.92 (dt, J=17.6, 11.8 Hz, 2H), 2.78 (d, J=11.5 Hz, 1H), 2.70 (d, J=11.7 Hz, 1H), 2.30 (s, 3H), 2.15 (td, J=11.7, 3.5 Hz, 1H), 1.91-1.83 (m, 1H), 1.37 (d, J=6.2 Hz, 3H).

### Example 276: (2R,6R)-4-(8-Cyano-[1,7]naphthyridin-5-yl)-6-methyl-morpholine-2-carboxylic acid ((R)-1-methyl-pyrrolidin-2-ylmethyl)-amide

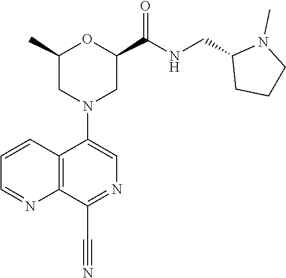 [SMILES: C[C@@H]1CN(c2cnc(C#N)c3ncccc23)C[C@H](C(=O)NC[C@H]2CCCN2C)O1]

The title compound was prepared from 5-[(2R,6R)-2-(hydroxymethyl)-6-methylmorpholin-4-yl]-1,7-naphthyridine-8-carbonitrile and [(2R)-1-methylpyrrolidin-2-yl]methanamine. MS: 395 [M+H]⁺. ¹H NMR (400 MHz, Methanol-d₄) δ 9.15 (d, J=4.1 Hz, 1H), 8.68 (d, J=8.7 Hz, 1H), 8.39 (s, 1H), 7.92-7.79 (m, 1H), 4.59-4.47 (m, 1H), 4.17 (p, J=7.0 Hz, 1H), 3.90-3.76 (m, 1H), 3.52 (dd, J=13.4, 4.0 Hz, 2H), 3.22 (dd, J=13.6, 6.4 Hz, 1H), 3.09 (dd, J=9.4, 4.9 Hz, 1H), 2.92 (dt, J=22.0, 11.3 Hz, 2H), 2.54-2.45 (m, 1H), 2.41 (d, J=1.6 Hz, 3H), 2.30 (q, J=8.9 Hz, 1H), 1.96 (dt, J=16.1, 8.0 Hz, 1H), 1.77 (p, J=7.3 Hz, 2H), 1.62 (dq, J=14.3, 7.4 Hz, 1H), 1.37 (d, J=5.9 Hz, 3H).

### Example 277: (2R,6R)-4-(8-Cyano-[1,7]naphthyridin-5-yl)-6-methyl-morpholine-2-carboxylic acid (4-morpholin-4-yl-cyclohexyl)-amide

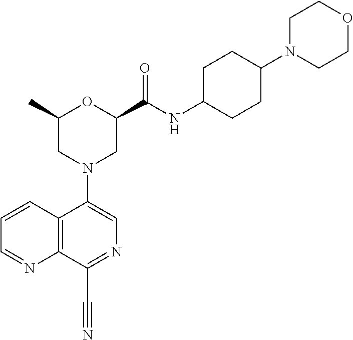 [SMILES: C[C@@H]1CN(c2cnc(C#N)c3ncccc23)C[C@H](C(=O)NC2CCC(N3CCOCC3)CC2)O1]

The title compound was prepared from 5-[(2R,6R)-2-(hydroxymethyl)-6-methylmorpholin-4-yl]-1,7-naphthyridine-8-carbonitrile and 4-Morpholin-4-yl-cyclohexylamine trifluoroacetate. MS: 465 [M+H]⁺. ¹H (400 MHz, Methanol-d₄) δ 9.15 (s, 1H), 8.67 (d, J=8.5 Hz, 1H), 8.39 (s, 1H), 7.86 (dt, J=7.9, 3.0 Hz, 1H), 4.47 (d, J=10.5 Hz, 1H), 4.16 (d, J=8.5 Hz, 1H), 3.82 (d, J=12.2 Hz, 1H), 3.71 (d, J=4.7 Hz, 5H), 3.52 (d, J=12.1 Hz, 1H), 2.92 (dt, J=21.7, 11.6 Hz, 2H), 2.61 (t, J=4.4 Hz, 4H), 2.28 (s, 1H), 2.10-1.89 (m, 4H), 1.54-1.24 (m, 7H).

### Example 278: (2R,6S)-4-(8-cyanoquinoxalin-5-yl)-N-[(2R)-2-hydroxypropyl]-6-(trifluoromethyl)morpholine-2-carboxamide & Example 279: (2S,6R)-4-(8-cyanoquinoxalin-5-yl)-N-[(2R)-2-hydroxypropyl]-6-(trifluoromethyl)morpholine-2-carboxamide

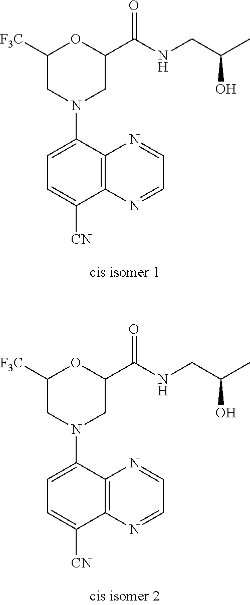 [SMILES: C[C@@H](O)CNC(=O)C1CN(c2ccc(C#N)c3nccnc23)CC(C(F)(F)F)O1 ; C[C@@H](O)CNC(=O)C1CN(c2ccc(C#N)c3nccnc23)CC(C(F)(F)F)O1]

The title compound was prepared from cis-4-(8-cyanoquinoxalin-5-yl)-6-(trifluoromethyl)morpholine-2-carboxylic acid and, followed by separation on chiral-HPLC under the following conditions: column, ChiralPAK IC-3, 0.46×10 cm, 3 um; mobile phase, hexane (with 0.1% FA) in EtOH, 50% isocratic in 20 min; detector, UV 254 nm.

Isomer 1: MS: 410 [M+H]⁺. ¹H NMR (400 MHz, Methanol-d₄, ppm) δ 8.99 (dd, J=17.4, 1.8 Hz, 2H), 8.20 (d, J=8.3 Hz, 1H), 7.36 (d, J=8.3 Hz, 1H), 4.62 (dd, J=10.9, 2.7 Hz, 1H), 4.58 (s, 1H), 4.48 (d, J=12.0 Hz, 2H), 3.90 (m, J=6.8, 4.3 Hz, 1H), 3.39 (dd, J=13.5, 4.3 Hz, 1H), 3.25-3.16 (m, 2H), 3.15-3.05 (m, 1H), 1.18 (d, J=6.3 Hz, 3H).

Isomer 2: MS: 410 [M+H]⁺. ¹H NMR (400 MHz, Methanol-d₄, ppm) δ 9.01-8.96 (m, 2H), 8.20 (d, J=8.3 Hz, 1H), 7.36 (d, J=8.2 Hz, 1H), 4.75-4.54 (m, 2H), 4.48 (d, J=12.3 Hz, 2H), 3.93-3.84 (m, 1H), 3.42-3.33 (m, 1H), 3.27-3.04 (m, 3H), 1.18 (d, J=6.3 Hz, 3H).

### Example 280: (2R,6S)-4-(8-cyanoquinoxalin-5-yl)-N-[(2S)-2,3-dihydroxypropyl]-6-(trifluoromethyl)morpholine-2-carboxamide & Example 281: (2S,6R)-4-(8-cyanoquinoxalin-5-yl)-N-[(2S)-2,3-dihydroxypropyl]-6-(trifluoromethyl)morpholine-2-carboxamide

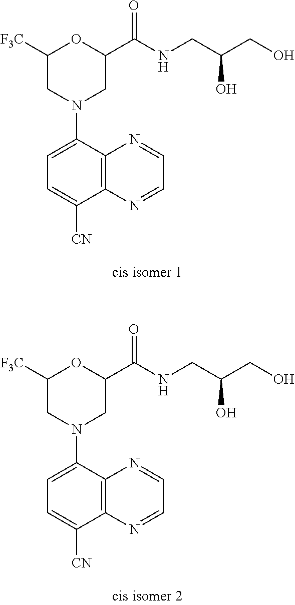 [SMILES: N#Cc1ccc(N2CC(C(=O)NC[C@H](O)CO)OC(C(F)(F)F)C2)c2nccnc12 ; N#Cc1ccc(N2CC(C(=O)NC[C@H](O)CO)OC(C(F)(F)F)C2)c2nccnc12]

The title compound was prepared from cis-4-(8-cyanoquinoxalin-5-yl)-6-(trifluoromethyl)morpholine-2-carboxylic acid and (2S)-3-aminopropane-1,2-diol, followed by separation on chiral-HPLC under the following conditions: column, CHIRALPAK ID-3, 0.46×5 cm, 3 um; mobile phase, hexane (with 0.1% DEA) in IPA, 50% isocratic in 20 min; detector, UV 254 nm.

Isomer 1: MS: 426 [M+H]⁺. ¹H NMR (400 MHz, DMSO-d₆, ppm) δ 9.10 (d, J=1.8 Hz, 1H), 9.03 (d, J=1.8 Hz, 1H), 8.30 (d, J=8.3 Hz, 1H), 7.69 (t, J=5.7 Hz, 1H), 7.39 (d, J=8.4 Hz, 1H), 4.85 (d, J=5.0 Hz, 1H), 4.74-4.70 (m, 1H), 4.60 (t, J=5.7 Hz, 1H), 4.57-4.49 (m, 1H), 4.44-4.34 (m, 2H), 3.59-3.49 (m, 1H), 3.39-3.15 (m, 4H), 3.15-3.03 (m, 2H).

Isomer 2: MS: 426 [M+H]⁺. ¹H NMR (400 MHz, DMSO-d₆, ppm) δ 9.10 (d, J=1.8 Hz, 1H), 9.03 (d, J=1.8 Hz, 1H), 8.30 (d, J=8.3 Hz, 1H), 7.68 (t, J=5.7 Hz, 1H), 7.39 (d, J=8.4 Hz, 1H), 4.85 (d, J=5.0 Hz, 1H), 4.72 (s, 1H), 4.60 (t, J=5.7 Hz, 1H), 4.53 (dd, J=10.9, 2.7 Hz, 1H), 4.39 (dd, J=10.1, 3.4 Hz, 1H), 3.55 (q, J=5.6 Hz, 1H), 3.39-3.15 (m, 3H), 3.15-3.01 (m, 2H).

### Example 282: (2R,6S)-4-(8-cyanoquinoxalin-5-yl)-N-[(2R)-2,3-dihydroxypropyl]-6-(trifluoromethyl)morpholine-2-carboxamide & Example 283: (2S,6R)-4-(8-cyanoquinoxalin-5-yl)-N-[(2R)-2,3-dihydroxypropyl]-6-(trifluoromethyl)morpholine-2-carboxamide

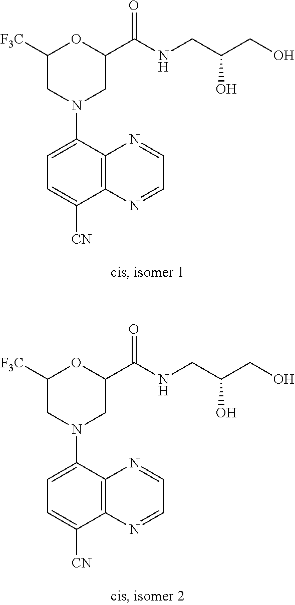 [SMILES: N#Cc1ccc(N2CC(C(=O)NC[C@@H](O)CO)OC(C(F)(F)F)C2)c2nccnc12 ; N#Cc1ccc(N2CC(C(=O)NC[C@@H](O)CO)OC(C(F)(F)F)C2)c2nccnc12]

The title compound was prepared from cis-4-(8-cyanoquinoxalin-5-yl)-6-(trifluoromethyl)morpholine-2-carboxylic acid and (2R)-3-aminopropane-1,2-diol, followed by separation on chiral-HPLC under the following conditions: column, CHIRALPAK IG-3, 0.46×5 cm, 3 um; mobile phase, hexane (with 20 mM NH₃.H₂O) in IPA, 50% isocratic in 20 min; detector, UV 254 nm.

Isomer 1: MS: 426 [M+H]⁺. ¹H NMR (300 MHz, DMSO-d₆, ppm) δ 9.10 (d, J=1.8 Hz, 1H), 9.03 (d, J=1.8 Hz, 1H), 8.30 (d, J=8.3 Hz, 1H), 7.71 (t, J=5.7 Hz, 1H), 7.39 (d, J=8.4 Hz, 1H), 4.87 (d, J=4.9 Hz, 1H), 4.78-4.67 (m, 1H), 4.62 (t, J=5.7 Hz, 1H), 4.58-4.48 (m, 1H), 4.44-4.33 (m, 2H), 3.60-3.49 (m, 1H), 3.30-3.01 (m, 6H).

Isomer 2: MS: 426 [M+H]⁺. ¹H NMR (300 MHz, DMSO-d₆, ppm) δ 9.08 (d, J=1.8 Hz, 1H), 9.01 (d, J=1.8 Hz, 1H), 8.28 (d, J=8.3 Hz, 1H), 7.69 (t, J=5.7 Hz, 1H), 7.37 (d, J=8.4 Hz, 1H), 4.85 (d, J=4.9 Hz, 1H), 4.80-4.67 (m, 1H), 4.60 (t, J=5.7 Hz, 1H), 4.50 (d, J=2.5 Hz, 1H), 4.42-4.22 (m, 2H), 3.57-3.45 (m, 1H), 3.31 (s, 1H), 3.25-2.85 (m, 5H).

### Example 284: (2R,6S)-4-(8-cyanoquinoxalin-5-yl)-N-(2-hydroxy-2-methylpropyl)-6-(trifluoromethyl)morpholine-2-carboxamide & Example 285: (2S,6R)-4-(8-cyanoquinoxalin-5-yl)-N-(2-hydroxy-2-methylpropyl)-6-(trifluoromethyl)morpholine-2-carboxamide

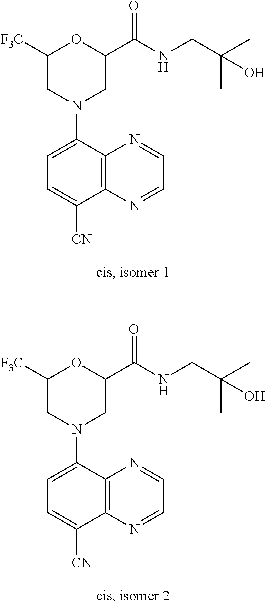 [SMILES: CC(C)(O)CNC(=O)C1CN(c2ccc(C#N)c3nccnc23)CC(C(F)(F)F)O1 ; CC(C)(O)CNC(=O)C1CN(c2ccc(C#N)c3nccnc23)CC(C(F)(F)F)O1]

The title compound was prepared from cis-4-(8-cyanoquinoxalin-5-yl)-6-(trifluoromethyl)morpholine-2-carboxylic acid and 1-amino-2-methylpropan-2-ol, followed by separation on chiral-HPLC under the following conditions: column, CHIRALPAK IE-3, 0.46×5 cm, 3 um; mobile phase, hexane (with 20 mM NH₃.H₂O) in IPA, 50% isocratic in 20 min; detector, UV 254 nm.

Isomer 1: MS: 424 [M+H]⁺. ¹H NMR (400 MHz, Methanol-d₄, ppm) δ 9.0 (d, J=1.8 Hz, 1H), 8.96 (d, J=1.8 Hz, 1H), 8.18 (d, J=8.3 Hz, 1H), 7.35 (d, J=8.3 Hz, 1H), 4.75-4.61 (m, 2H), 4.54-4.43 (m, 2H), 3.33-3.28 (m, 2H), 3.28-3.05 (m, 2H), 1.22 (s, 3H), 1.21 (s, 3H).

Isomer 2: MS: 424 [M+H]⁺. ¹H NMR (400 MHz, Methanol-d₄, ppm) δ 8.98 (dd, J=16.6, 1.8 Hz, 2H), 8.18 (d, J=8.3 Hz, 1H), 7.35 (d, J=8.3 Hz, 1H), 4.75-4.61 (m, 2H), 4.54-4.43 (m, 2H), 3.31 (s, 2H), 3.22 (dd, J=12.2, 10.8 Hz, 1H), 3.10 (dd, J=12.6, 10.8 Hz, 1H), 1.22 (d, J=2.8 Hz, 6H).

### Example 286: (2R,6S)-4-(8-cyanoquinoxalin-5-yl)-N—((S)-2-hydroxypropyl)-6-(trifluoromethyl)morpholine-2-carboxamide & Example 287: (2S,6R)-4-(8-cyanoquinoxalin-5-yl)-N—((S)-2-hydroxypropyl)-6-(trifluoromethyl)morpholine-2-carboxamide

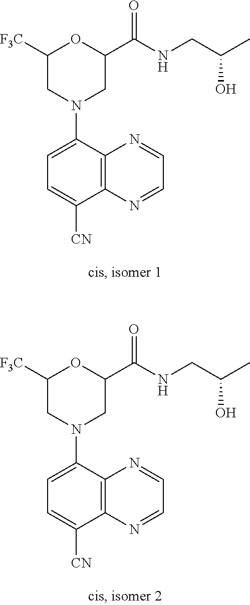 [SMILES: C[C@H](O)CNC(=O)C1CN(c2ccc(C#N)c3nccnc23)CC(C(F)(F)F)O1 ; C[C@H](O)CNC(=O)C1CN(c2ccc(C#N)c3nccnc23)CC(C(F)(F)F)O1]

The title compound was prepared from cis-4-(8-cyanoquinoxalin-5-yl)-6-(trifluoromethyl)morpholine-2-carboxylic acid and (2S)-1-aminopropan-2-ol, followed by separation on chiral-HPLC under the following conditions: column, CHIRALPAK IA, 0.46×10 cm, 3 um; mobile phase, MtBE (with 0.1% DEA) in EtOH, 70% isocratic in 20 min; detector, UV 254 nm.

Isomer 1: MS: 410 [M+H]⁺. ¹H NMR (400 MHz, DMSO-d₆, ppm) δ 9.08 (d, J=1.8 Hz, 1H), 9.03 (d, J=1.8 Hz, 1H), 8.29 (d, J=8.3 Hz, 1H), 7.67 (t, J=5.8 Hz, 1H), 7.37 (d, J=8.4 Hz, 1H), 4.79-4.67 (m, 2H), 4.57-4.49 (m, 1H), 4.44-4.34 (m, 2H), 3.76-3.65 (m, 1H), 3.24-2.95 (m, 4H), 1.01 (d, J=6.1 Hz, 3H).

Isomer 2: MS: 410 [M+H]⁺. ¹H NMR (400 MHz, DMSO-d₆, ppm) δ 9.10 (d, J=1.8 Hz, 1H), 9.03 (d, J=1.8 Hz, 1H), 8.28 (d, J=8.2 Hz, 1H), 7.66 (t, J=5.8 Hz, 1H), 7.39 (d, J=8.4 Hz, 1H), 4.79-4.67 (m, 2H), 4.57-4.49 (m, 1H), 4.44-4.34 (m, 2H), 3.76-3.65 (m, 1H), 3.26-2.97 (m, 4H), 1.03 (d, J=6.2 Hz, 3H).

### Example 288: 8-[(2R,6R)-2-((S)-7-Amino-5-aza-spiro[2.4]heptane-5-carbonyl)-6-methyl-morpholin-4-yl]-quinoxaline-5-carbonitrile

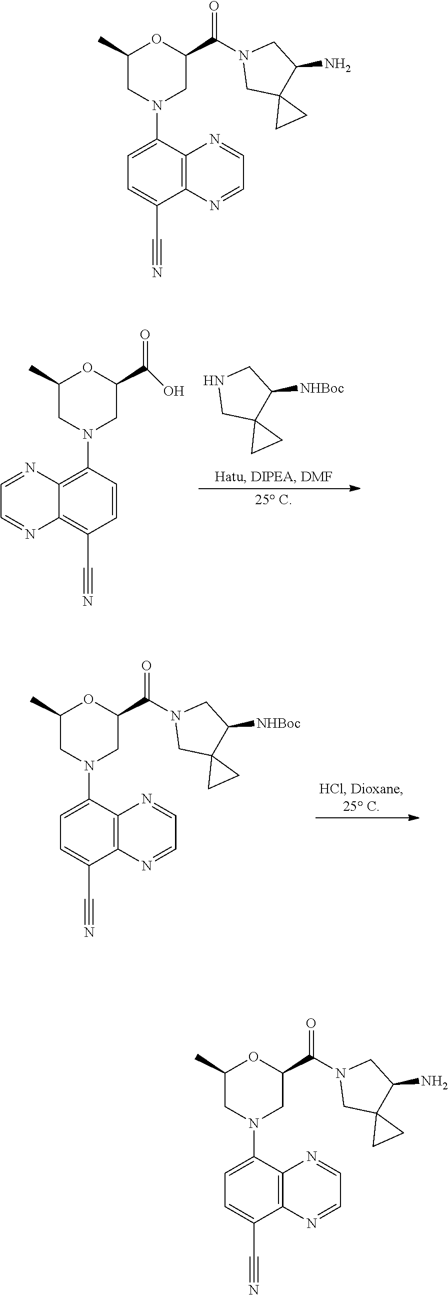 [SMILES: C[C@@H]1CN(c2ccc(C#N)c3nccnc23)C[C@H](C(=O)N2C[C@@H](N)C3(CC3)C2)O1 ; C[C@@H]1CN(c2ccc(C#N)c3nccnc23)C[C@H](C(=O)O)O1 ; CC(C)(C)OC(=O)N[C@@H]1CNCC12CC2 ; C[C@@H]1CN(c2ccc(C#N)c3nccnc23)C[C@H](C(=O)N2C[C@@H](NC(=O)OC(C)(C)C)C3(CC3)C2)O1 ; C[C@@H]1CN(c2ccc(C#N)c3nccnc23)C[C@H](C(=O)N2C[C@@H](N)C3(CC3)C2)O1]

{(S)-5-[(2R,6R)-4-(8-Cyano-quinoxalin-5-yl)-6-methyl-morpholine-2-carbonyl]-5-aza-spiro[2.4]hept-7-yl}-carbamic acid tert-butyl ester: Into a 50 mL round-bottom flask, was placed (2R,6R)-4-(8-Cyano-quinoxalin-5-yl)-6-methyl-morpholine-2-carboxylic acid (80.0 mg; 0.27 mmol; 1.0 eq.) in DMF (2.0 ml), hatu (203.95 mg; 0.54 mmol; 2.0 eq.) was added and the resulting solution stirred for 10 minutes at rt after which (S)-(5-Aza-spiro[2.4]hept-7-yl)-carbamic acid tert-butyl ester (68.32 mg; 0.32 mmol; 1.20 eq.) and DIPEA (0.14 ml; 0.80 mmol; 3.0 eq.) were respectively added. The resulting mixture was stirred at room temperature for 2 h. The volatils were evaporated and the residue dissolved in DCM (2 mL). The solution was absorbed on a PuriFlash 12 g column and purified by chromatography (Hexane-Ethyl acetate, gradient 80-20% for 5 minutes then 30-70% for 25 minutes to yield the title compound (75.0 mg; 56%) as a yellow oil. MS:493 [M+H]⁺.

8-[(2R,6R)-2-((S)-7-Amino-5-aza-spiro[2.4]heptane-5-carbonyl)-6-methyl-morpholin-4-yl]-quinoxaline-5-carbonitrile: {(S)-5-[(2R,6R)-4-(8-Cyano-quinoxalin-5-yl)-6-methyl-morpholine-2-carbonyl]-5-aza-spiro[2.4]hept-7-yl}-carbamic acid tert-butyl ester (49.31 mg; 0.10 mmol; 1.0 eq.) was suspended in Dioxane (0.2 mL). Hydrochloric Acid in dioxane (0.25 ml; 1.0 mmol; 10.0 eq.) was added to suspension dropwise, which became homogenous solution upon addition. The resolution solution was stirred for 4 hours. The volatils were evaporated and the residue dissolved in 4 mL of DMSO. The product was purified on reverse phase system using a gradient of 05-95% CH₃CN/H₂O (0.1% Ammonium Hydroxide) in 4 injections of 1 mL each. The desired fractions were evaporated to provide the title compound (12.50 mg; 30%) as a yellow solid. MS: 393 [M+H]⁺. ¹H NMR (400 MHz, Methanol-d₄) δ 8.96 (d, J=1.6 Hz, 1H), 8.90 (dd, J=19.5, 1.7 Hz, 1H), 8.14 (d, J=8.3 Hz, 1H), 7.26 (dd, J=8.4, 3.5 Hz, 1H), 4.70 (ddd, J=21.3, 10.5, 2.4 Hz, 1H), 4.47 (dd, J=12.4, 6.5 Hz, 1H), 4.14-3.95 (m, 3H), 3.82-3.71 (m, 1H), 3.65 (t, J=11.3 Hz, 1H), 3.57-3.44 (m, 1H), 3.21-3.05 (m, 2H), 2.91-2.81 (m, 1H), 1.32 (dd, J=11.5, 6.2 Hz, 3H), 0.91 (dd, J=9.9, 4.2 Hz, 1H), 0.76-0.59 (m, 3H).

The following compounds were synthesized in an analogous manner:

### Example 289: (2R,6R)-4-(8-Cyano-quinoxalin-5-yl)-6-methyl-morpholine-2-carboxylic acid (3,3-difluoro-piperidin-4-yl)-amide

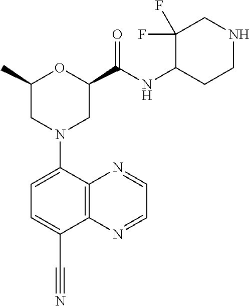 [SMILES: C[C@@H]1CN(c2ccc(C#N)c3nccnc23)C[C@H](C(=O)NC2CCNCC2(F)F)O1]

The title compound was prepared from (2R,6R)-4-(8-Cyano-quinoxalin-5-yl)-6-methyl-morpholine-2-carboxylic acid and tert-butyl 4-amino-3,3-difluoropiperidine-1-carboxylate. MS: 417 [M+H]⁺. ¹H NMR (400 MHz, Methanol-d₄) δ 8.97 (d, J=1.8 Hz, 1H), 8.93 (d, J=1.8 Hz, 1H), 8.14 (d, J=8.3 Hz, 1H), 7.25 (dd, J=8.4, 2.0 Hz, 1H), 4.62-4.46 (m, 2H), 4.46-4.33 (m, 1H), 4.14 (ddt, J=12.0, 10.0, 3.1 Hz, 2H), 3.22 (d, J=3.1 Hz, 1H), 3.10-3.02 (m, 1H), 2.99-2.81 (m, 3H), 2.72 (tdd, J=12.1, 3.2, 1.7 Hz, 1H), 1.99-1.88 (m, 1H), 1.87-1.70 (m, 1H), 1.37 (dd, J=6.2, 1.8 Hz, 3H).

### Example 290: (2R,6R)-4-(8-Cyano-quinoxalin-5-yl)-6-methyl-morpholine-2-carboxylic acid (4-fluoro-piperidin-4-ylmethyl)-amide

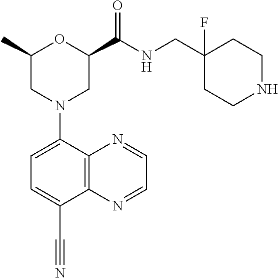 [SMILES: C[C@@H]1CN(c2ccc(C#N)c3nccnc23)C[C@H](C(=O)NCC2(F)CCNCC2)O1]

The title compound was prepared from (2R,6R)-4-(8-Cyano-quinoxalin-5-yl)-6-methyl-morpholine-2-carboxylic acid and tert-butyl 4-(aminomethyl)-4-fluoropiperidine-1-carboxylate. MS: 413 [M+H]⁺. ¹H NMR (400 MHz, Methanol-d₄) δ 8.97 (s, 1H), 8.93 (s, 1H), 8.14 (dd, J=8.5, 1.7 Hz, 1H), 7.26 (d, J=8.3 Hz, 1H), 4.58 (dd, J=12.1, 2.5 Hz, 1H), 4.49 (dt, J=10.7, 2.3 Hz, 1H), 4.14 (t, J=10.2 Hz, 2H), 3.57-3.40 (m, 2H), 3.0-2.79 (m, 6H), 1.89-1.55 (m, 4H), 1.37 (d, J=6.1 Hz, 3H).

### Example 291: (2R,6R)-4-(8-Cyano-quinoxalin-5-yl)-6-methyl-morpholine-2-carboxylic acid (5-aza-spiro[3.5]non-8-yl)-amide

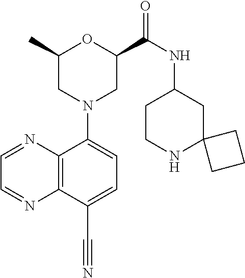 [SMILES: C[C@@H]1CN(c2ccc(C#N)c3nccnc23)C[C@H](C(=O)NC2CCNC3(CCC3)C2)O1]

The title compound was prepared from (2R,6R)-4-(8-Cyano-quinoxalin-5-yl)-6-methyl-morpholine-2-carboxylic acid and tert-butyl 8-amino-5-azaspiro[3.5]nonane-5-carboxylate. MS: 421 [M+H]⁺. ¹H NMR (400 MHz, Methanol-d₄) δ 8.97 (s, 1H), 8.92 (s, 1H), 8.14 (d, J=8.3 Hz, 1H), 7.25 (d, J=8.3 Hz, 1H), 4.54 (d, J=12.3 Hz, 1H), 4.41 (d, J=10.7 Hz, 1H), 4.19-4.04 (m, 2H), 4.0-3.87 (m, 1H), 2.93 (q, J=12.0 Hz, 2H), 2.81 (q, J=12.2, 11.5 Hz, 2H), 2.30-1.76 (m, 8H), 1.57-1.41 (m, 2H), 1.36 (d, J=6.2 Hz, 3H).

### Example 292: (2R,6R)-4-(8-Cyano-quinoxalin-5-yl)-6-methyl-morpholine-2-carboxylic acid (3-fluoro-pyrrolidin-3-ylmethyl)-amide

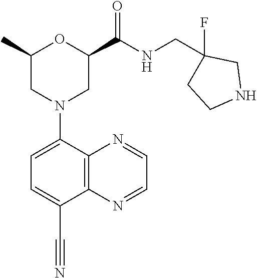 [SMILES: C[C@@H]1CN(c2ccc(C#N)c3nccnc23)C[C@H](C(=O)NCC2(F)CCNC2)O1]

The title compound was prepared from (2R,6R)-4-(8-Cyano-quinoxalin-5-yl)-6-methyl-morpholine-2-carboxylic acid and tert-butyl 3-(aminomethyl)-3-fluoropyrrolidine-1-carboxylate. MS: 399 [M+H]⁺. ¹H NMR (400 MHz, Methanol-d₄) δ 8.97 (s, 1H), 8.93 (s, 1H), 8.14 (d, J=8.3 Hz, 1H), 7.26 (d, J=8.3 Hz, 1H), 4.57 (d, J=12.3 Hz, 1H), 4.48 (d, J=11.1 Hz, 1H), 4.13 (t, J=10.4 Hz, 2H), 3.68 (d, J=19.6 Hz, 2H), 3.19-2.89 (m, 5H), 2.84 (t, J=11.6 Hz, 1H), 2.0 (dtd, J=45.3, 16.6, 14.5, 8.9 Hz, 2H), 1.36 (d, J=6.1 Hz, 3H).

### Example 293: (2R,6R)-4-(8-Cyano-quinoxalin-5-yl)-6-methyl-morpholine-2-carboxylic acid (3-fluoro-azetidin-3-ylmethyl)-amide

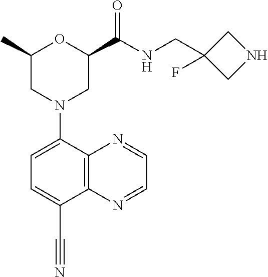 [SMILES: C[C@@H]1CN(c2ccc(C#N)c3nccnc23)C[C@H](C(=O)NCC2(F)CNC2)O1]

The title compound was prepared from (2R,6R)-4-(8-Cyano-quinoxalin-5-yl)-6-methyl-morpholine-2-carboxylic acid and 3-(aminomethyl)-3-fluoro-1-boc-azetidine. MS: 385 [M+H]⁺. ¹H NMR (400 MHz, Methanol-d₄) δ 8.97 (d, J=1.5 Hz, 1H), 8.93 (d, J=1.6 Hz, 1H), 8.15 (dd, J=8.4, 1.2 Hz, 1H), 7.26 (d, J=8.3 Hz, 1H), 4.58 (d, J=12.2 Hz, 1H), 4.52-4.45 (m, 1H), 4.19-4.07 (m, 2H), 3.84-3.62 (m, 6H), 2.94 (t, J=11.4 Hz, 1H), 2.84 (t, J=11.1 Hz, 1H), 1.36 (d, J=6.1 Hz, 3H).

### Example 294: (2R,6R)-4-(8-Cyano-quinoxalin-5-yl)-6-methyl-morpholine-2-carboxylic acid ((3S,4R)-4-fluoro-pyrrolidin-3-yl)-amide hydrochloride (2)

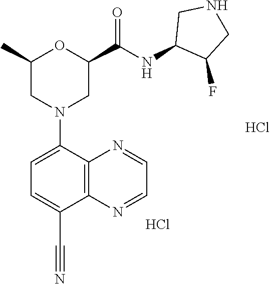 [SMILES: C[C@@H]1CN(c2ccc(C#N)c3nccnc23)C[C@H](C(=O)N[C@H]2CNC[C@H]2F)O1 ; Cl ; Cl]

The title compound was prepared from (2R,6R)-4-(8-Cyano-quinoxalin-5-yl)-6-methyl-morpholine-2-carboxylic acid and (3s,4r)-tert-butyl 3-amino-4-fluoropyrrolidine-1-carboxylate, and isolated as HCl salt. MS: 385 [M+H]⁺. ¹H NMR (400 MHz, DMSO-d₆) δ 9.84 (s, 1H), 9.60 (s, 1H), 9.08 (d, J=1.8 Hz, 1H), 9.0 (d, J=1.8 Hz, 1H), 8.26 (d, J=8.4 Hz, 1H), 8.15 (d, J=7.6 Hz, 1H), 7.29 (d, J=8.4 Hz, 1H), 5.29-5.10 (m, 1H), 4.59-4.44 (m, 2H), 4.39-4.34 (m, 1H), 4.13 (dt, J=12.5, 2.2 Hz, 1H), 3.98 (ddd, J=10.4, 6.2, 2.4 Hz, 1H), 3.63-3.45 (m, 4H), 3.02-2.95 (m, 1H), 2.84 (dd, J=12.5, 10.4 Hz, 1H), 1.27 (d, J=6.2 Hz, 3H).

### Example 295: 8-[(2R,6R)-2-((3R,5S)-3-Amino-5-trifluoromethyl-piperidine-1-carbonyl)-6-methyl-morpholin-4-yl]-quinoxaline-5-carbonitrile hydrochloride (2)

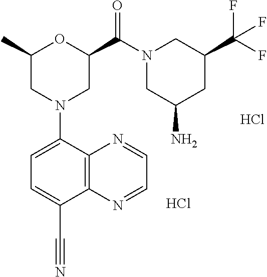 [SMILES: C[C@@H]1CN(c2ccc(C#N)c3nccnc23)C[C@H](C(=O)N2C[C@H](N)C[C@H](C(F)(F)F)C2)O1 ; Cl ; Cl]

The title compound was prepared from (2R,6R)-4-(8-Cyano-quinoxalin-5-yl)-6-methyl-morpholine-2-carboxylic acid and tert-butyl N-[(3R,5S)-5-(trifluoromethyl)piperidin-3-yl]carbamate, and isolated as HCl salt. MS: 449 [M+H]⁺. ¹H NMR (400 MHz, Methanol-d₄) δ 8.97 (s, 1H), 8.90 (s, 1H), 8.14 (d, J=8.2 Hz, 1H), 7.27 (d, J=8.3 Hz, 1H), 4.92-4.76 (m, 1H), 4.62-4.46 (m, 1H), 4.38 (d, J=12.8 Hz, 1H), 4.27-4.09 (m, 3H), 3.19 (dt, J=24.3, 13.1 Hz, 4H), 2.83 (d, J=22.4 Hz, 1H), 2.65 (dd, J=40.4, 12.0 Hz, 3H), 2.45 (t, J=14.4 Hz, 2H), 1.81-1.64 (m, 2H), 1.32 (d, J=6.0 Hz, 3H).

### Example 296: (2R,6R)-4-(8-cyanoquinoxalin-5-yl)-6-methyl-N-{[(3S)-morpholin-3-yl]methyl}morpholine-2-carboxamide & Example 297: (2R,6R)-4-(8-cyanoquinoxalin-5-yl)-6-methyl-N-{[(3R)-morpholin-3-yl]methyl}morpholine-2-carboxamide

The title compounds were made from (2R,6R)-4-(8-Cyano-quinoxalin-5-yl)-6-methyl-morpholine-2-carboxylic acid and tert-butyl 3-(aminomethyl)morpholine-4-carboxylate, and were separated by SFC. The conditions are: column, ADH, Prep SFC-P100; mobile phase, methanol+20 mM NH₄OH, 45° C./80 bar, 100 g/min; detector, PDA. The configurations of the structures were tentatively assigned.

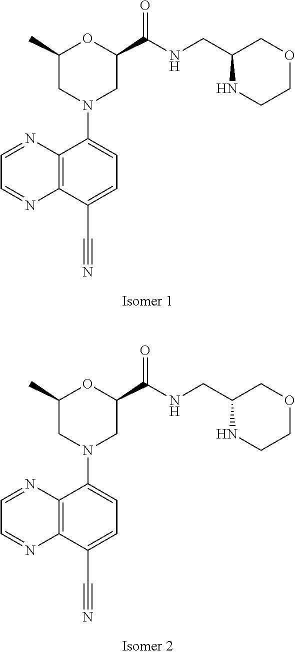 [SMILES: C[C@@H]1CN(c2ccc(C#N)c3nccnc23)C[C@H](C(=O)NC[C@H]2COCCN2)O1 ; C[C@@H]1CN(c2ccc(C#N)c3nccnc23)C[C@H](C(=O)NC[C@@H]2COCCN2)O1]

Isomer 1: MS:397 [M+H]⁺. ¹H NMR (400 MHz, DMSO-d₆) δ 9.07 (d, J=1.8 Hz, 1H), 9.0 (d, J=1.8 Hz, 1H), 8.25 (d, J=8.4 Hz, 1H), 7.77 (t, J=5.9 Hz, 1H), 7.28 (d, J=8.4 Hz, 1H), 4.48 (dt, J=12.5, 2.3 Hz, 1H), 4.29 (dd, J=10.8, 2.7 Hz, 1H), 4.15-4.08 (m, 1H), 3.97 (ddd, J=10.5, 6.2, 2.4 Hz, 1H), 3.62 (dt, J=12.8, 3.3 Hz, 2H), 3.34 (dd, J=10.6, 2.9 Hz, 1H), 3.08-3.02 (m, 2H), 2.93 (dd, J=12.4, 10.8 Hz, 1H), 2.84-2.73 (m, 3H), 2.73-2.64 (m, 1H), 1.27 (d, J=6.2 Hz, 3H).

Isomer 2: MS:397 [M+H]⁺. ¹H NMR (400 MHz, DMSO-d₆) δ 9.08 (d, J=1.8 Hz, 1H), 9.0 (d, J=1.8 Hz, 1H), 8.25 (d, J=8.4 Hz, 1H), 7.78 (t, J=6.0 Hz, 1H), 7.28 (d, J=8.4 Hz, 1H), 4.48 (dt, J=12.4, 2.4 Hz, 1H), 4.29 (dd, J=10.8, 2.7 Hz, 1H), 4.15-4.08 (m, 1H), 3.97 (ddd, J=10.4, 6.2, 2.3 Hz, 1H), 3.68-3.58 (m, 2H), 3.39-3.25 (m, 1H), 3.07 (td, J=8.3, 1.8 Hz, 2H), 2.93 (dd, J=12.4, 10.8 Hz, 1H), 2.89-2.71 (m, 3H), 2.71-2.64 (m, 1H), 1.27 (d, J=6.2 Hz, 3H).

### Example 298: (2R,6R)-4-(7-Fluoro-8-methyl-quinolin-5-yl)-6-methyl-morpholine-2-carboxylic acid ((3S,4R)-4-fluoro-pyrrolidin-3-yl)-amide

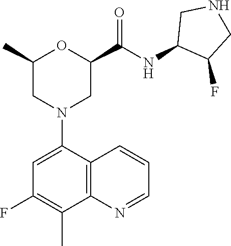 [SMILES: Cc1c(F)cc(N2C[C@@H](C)O[C@@H](C(=O)N[C@H]3CNC[C@H]3F)C2)c2cccnc12]

The title compound was prepared from (2R,6R)-4-(7-Fluoro-8-methyl-quinolin-5-yl)-6-methyl-morpholine-2-carboxylic acid and (3s,4r)-tert-butyl 3-amino-4-fluoropyrrolidine-1-carboxylate. MS: 391 [M+H]⁺. ¹H NMR (400 MHz, Methanol-d₄) δ 8.90 (d, J=4.4 Hz, 1H), 8.65 (d, J=8.5 Hz, 1H), 7.52 (dd, J=8.5, 4.3 Hz, 1H), 7.09 (d, J=11.2 Hz, 1H), 5.18-4.99 (m, 1H), 4.57-4.49 (m, 1H), 4.35 (dtd, J=25.0, 8.6, 4.5 Hz, 1H), 4.20-4.11 (m, 1H), 3.89-3.63 (m, 1H), 3.56 (d, J=12.0 Hz, 1H), 3.29-3.08 (m, 3H), 2.73 (ddt, J=29.3, 22.9, 10.8 Hz, 3H), 2.60 (s, 3H), 1.34 (d, J=6.2 Hz, 3H).

### Example 299: (2R,6R)-4-(8-Cyano-quinazolin-5-yl)-6-methyl-morpholine-2-carboxylic acid (3,3-difluoro-piperidin-4-yl)-amide

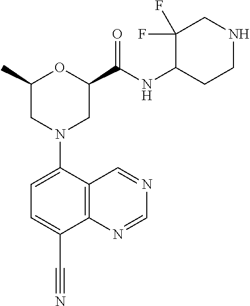 [SMILES: C[C@@H]1CN(c2ccc(C#N)c3ncncc23)C[C@H](C(=O)NC2CCNCC2(F)F)O1]

The title compound was prepared from (2R,6R)-4-(8-Cyano-quinazolin-5-yl)-6-methyl-morpholine-2-carboxylic acid and tert-butyl 4-amino-3,3-difluoropiperidine-1-carboxylate, followed by de-Boc with TFA in DCM and purified by reverse phase system using a gradient of 5-95% ACN/water (0.1% Ammonium Hydroxide). MS: 417 [M+H]⁺. ¹H NMR (400 MHz, Methanol-d₄) δ 9.75 (d, J=2.4 Hz, 1H), 9.35 (d, J=1.9 Hz, 1H), 8.41-8.26 (m, 1H), 7.35 (d, J=8.2 Hz, 1H), 4.59 (t, J=9.6 Hz, 1H), 4.49-4.32 (m, 1H), 4.26-4.15 (m, 1H), 3.90-3.81 (m, 1H), 3.59 (d, J=12.4 Hz, 1H), 3.22 (t, J=12.9 Hz, 1H), 2.96 (dq, J=60.6, 16.2, 14.3 Hz, 4H), 2.72 (t, J=13.0 Hz, 1H), 1.93 (dt, J=16.0, 8.1 Hz, 1H), 1.82-1.69 (m, 1H), 1.37 (d, J=6.2 Hz, 3H).

### Example 300: (2R,6R)-4-(8-Cyano-[1,7]naphthyridin-5-yl)-6-methyl-morpholine-2-carboxylic acid (4-fluoro-piperidin-4-ylmethyl)-amide

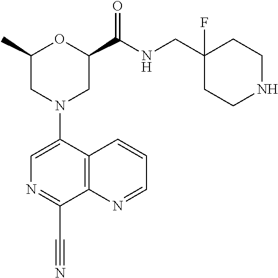 [SMILES: C[C@@H]1CN(c2cnc(C#N)c3ncccc23)C[C@H](C(=O)NCC2(F)CCNCC2)O1]

The title compound was prepared from (2R,6R)-4-(8-Cyano-[1,7]naphthyridin-5-yl)-6-methyl-morpholine-2-carboxylic acid and tert-butyl 4-(aminomethyl)-4-fluoropiperidine-1-carboxylate, followed by de-Boc with TFA in DCM and purified by reverse phase system using a gradient of 5-95% ACN/water (0.1% Ammonium Hydroxide). MS: 413 [M+H]⁺. ¹H NMR (400 MHz, Methanol-d₄) 9.15 (d, J=4.1 Hz, 1H), 8.68 (d, J=8.6 Hz, 1H), 8.40 (s, 1H), 7.86 (dd, J=8.7, 4.1 Hz, 1H), 4.56 (dd, J=10.9, 2.5 Hz, 1H), 4.18 (t, J=8.0 Hz, 1H), 3.91-3.72 (m, 1H), 3.60-3.40 (m, 3H), 3.07-2.77 (m, 6H), 1.90-1.49 (m, 4H), 1.38 (d, J=6.2 Hz, 3H).

### Example 301: (2R,6R)-4-(8-Cyano-[1,7]naphthyridin-5-yl)-6-methyl-morpholine-2-carboxylic acid (3-fluoro-azetidin-3-ylmethyl)-amide

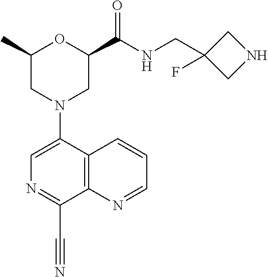 [SMILES: C[C@@H]1CN(c2cnc(C#N)c3ncccc23)C[C@H](C(=O)NCC2(F)CNC2)O1]

The title compound was prepared from (2R,6R)-4-(8-Cyano-quinazolin-5-yl)-6-methyl-morpholine-2-carboxylic acid and 3-(aminomethyl)-3-fluoro-1-boc-azetidine, followed by de-Boc with TFA in DCM and purified by reverse phase system using a gradient of 5-95% ACN/water (0.1% Ammonium Hydroxide). MS: 385 [M+H]⁺. ¹H NMR (400 MHz, Methanol-d₄) δ 9.15 (d, J=4.1 Hz, 1H), 8.68 (d, J=8.6 Hz, 1H), 8.40 (d, J=1.1 Hz, 1H), 7.86 (dd, J=8.7, 4.2 Hz, 1H), 4.67-4.45 (m, 2H), 4.18 (dt, J=12.0, 7.1 Hz, 1H), 3.89-3.60 (m, 6H), 3.53 (d, J=12.0 Hz, 1H), 2.94 (dt, J=26.7, 11.5 Hz, 2H), 1.38 (d, J=6.2 Hz, 3H).

### Example 302: (2R,6R)-4-(8-Cyano-[1,7]naphthyridin-5-yl)-6-methyl-morpholine-2-carboxylic acid (3,3-difluoro-piperidin-4-yl)-amide

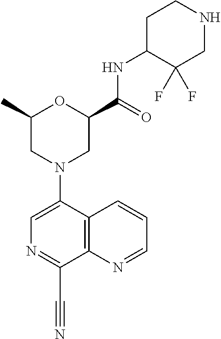 [SMILES: C[C@@H]1CN(c2cnc(C#N)c3ncccc23)C[C@H](C(=O)NC2CCNCC2(F)F)O1]

The title compound was prepared from (2R,6R)-4-(8-Cyano-quinazolin-5-yl)-6-methyl-morpholine-2-carboxylic acid and tert-butyl 4-amino-3,3-difluoropiperidine-1-carboxylate, followed by de-Boc with TFA in DCM and purified by reverse phase system using a gradient of 5-95% ACN/water (0.1% Ammonium Hydroxide). MS: 417 [M+H]⁺. ¹H NMR (400 MHz, Methanol-d₄) δ 9.14 (dd, J=4.2, 1.5 Hz, 1H), 8.67 (dt, J=8.7, 1.8 Hz, 1H), 8.39 (d, J=2.7 Hz, 1H), 7.94-7.82 (m, 1H), 4.58 (ddd, J=11.0, 8.4, 2.7 Hz, 1H), 4.40 (ddt, J=22.3, 12.2, 4.6 Hz, 1H), 4.27-4.11 (m, 1H), 3.83 (ddt, J=11.7, 9.0, 2.3 Hz, 1H), 3.53 (dt, J=12.2, 2.2 Hz, 1H), 3.25 (t, J=7.2 Hz, 1H), 3.12-2.82 (m, 4H), 2.74 (t, J=12.9 Hz, 1H), 2.02-1.89 (m, 1H), 1.85-1.72 (m, 1H), 1.39 (d, J=6.3, 1.3 Hz, 3H).

### Example 303 (Isomer 1): (2R,6S)-4-(8-cyanoquinoxalin-5-yl)-N-[(3S,4R)-4-fluoropyrrolidin-3-yl]-6-(trifluoromethyl)morpholine-2-carboxamide & Example 304 (Isomer 2): (2S,6R)-4-(8-cyanoquinoxalin-5-yl)-N-[(3S,4R)-4-fluoropyrrolidin-3-yl]-6-(trifluoromethyl)morpholine-2-carboxamide

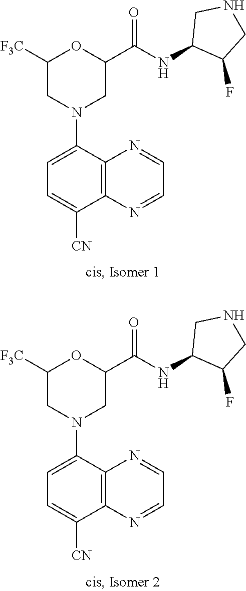 [SMILES: N#Cc1ccc(N2CC(C(=O)N[C@H]3CNC[C@H]3F)OC(C(F)(F)F)C2)c2nccnc12 ; N#Cc1ccc(N2CC(C(=O)N[C@H]3CNC[C@H]3F)OC(C(F)(F)F)C2)c2nccnc12]

The title compounds were prepared from cis-4-(8-cyanoquinoxalin-5-yl)-6-(trifluoromethyl)morpholine-2-carboxylic acid and tert-butyl (3S,4R)-3-amino-4-fluoropyrrolidine-1-carboxylate. The two isomers were obtained by separation on chiral-HPLC under the following conditions: column CHIRALPAK ID-3, 0.46×5 cm, 3 um; mobile phase, hexane (with 20 mM NH₃H₂O) in IPA, 50% isocratic in 20 min; detector, UV 254 nm.

Isomer 1: MS: 439 [M+H]⁺. ¹H NMR (400 MHz, Methanol-d₄, ppm) δ 8.99 (dd, J=17.9, 1.8 Hz, 2H), 8.19 (d, J=8.2 Hz, 1H), 7.35 (d, J=8.3 Hz, 1H), 5.11 (dt, J=59.6, 4.0 Hz, 1H), 4.71-4.62 (m, 2H), 4.51-4.32 (m, 3H), 3.29-3.09 (m, 5H), 2.87-2.77 (m, 1H).

Isomer 2: MS: 439 [M+H]⁺. ¹H NMR (400 MHz, Methanol-d₄, ppm) δ 9.03-8.94 (m, 2H), 8.19 (d, J=8.3, 1.7 Hz, 1H), 7.35 (d, J=8.3, 1.3 Hz, 1H), 5.14 (dt, J=55.2, 4.0 Hz, 1H), 4.72-4.62 (m, 2H), 4.53-4.30 (m, 3H), 3.33-3.08 (m, 5H), 2.87-2.77 (m, 1H).

### Example 305 (Isomer 1): (2R,6S)-4-(8-cyanoquinoxalin-5-yl)-N-[(4-fluoropiperidin-4-yl)methyl]-6-(trifluoromethyl)morpholine-2-carboxamide & Example 306 (Isomer 2): (2S,6R)-4-(8-cyanoquinoxalin-5-yl)-N-[(4-fluoropiperidin-4-yl)methyl]-6-(trifluoromethyl)morpholine-2-carboxamide

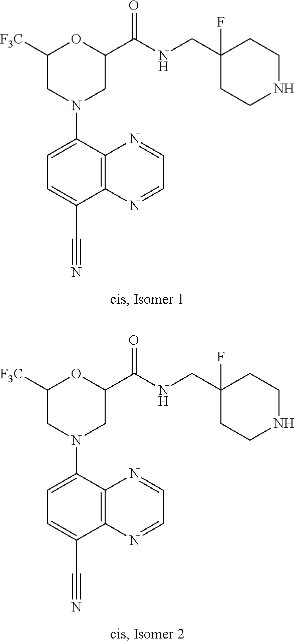 [SMILES: N#Cc1ccc(N2CC(C(=O)NCC3(F)CCNCC3)OC(C(F)(F)F)C2)c2nccnc12 ; N#Cc1ccc(N2CC(C(=O)NCC3(F)CCNCC3)OC(C(F)(F)F)C2)c2nccnc12]

The title compounds were prepared from cis-4-(8-cyanoquinoxalin-5-yl)-6-(trifluoromethyl)morpholine-2-carboxylic acid and tert-butyl 4-(aminomethyl)-4-fluoropiperidine-1-carboxylate. The two isomers were obtained by separation on chiral-HPLC under the following conditions: column, CHIRALPAK Cellulose-SB, 0.46×15 cm, 3 um; mobile phase, MtBE (with 0.1% DEA) in EtOH, 70% isocratic in 20 min; detector, UV 254 nm.

Isomer 1: MS: 467 [M+H]⁺. ¹H NMR (400 MHz, DMSO-d₆, ppm) δ 9.10 (d, J=1.8 Hz, 1H), 9.03 (d, J=1.8 Hz, 1H), 8.30 (d, J=8.3 Hz, 1H), 7.99-7.91 (m, 1H), 7.40 (d, J=8.4 Hz, 1H), 4.77-4.65 (m, 1H), 4.61-4.53 (m, 1H), 4.44-4.32 (m, 2H), 3.43-3.36 (m, 2H), 3.29-3.05 (m, 3H), 2.85-2.59 (m, 4H), 1.74-1.37 (m, 4H).

Isomer 2: MS: 467 [M+H]⁺. ¹H NMR (400 MHz, DMSO-d₆, ppm) δ 9.04 (m, J=29.2, 1.9 Hz, 2H), 8.27 (dd, J=8.3, 3.8 Hz, 1H), 7.96 (m, J=6.4 Hz, 1H), 7.37 (dd, J=8.7, 3.1 Hz, 1H), 4.80-4.64 (m, 1H), 4.56 (dd, J=10.8, 2.6 Hz, 1H), 4.36 (m, J=14.1 Hz, 2H), 3.36 (m, J=20.9, 6.4, 3.2 Hz, 3H), 3.17 (m, J=33.3, 11.7 Hz, 2H), 2.84-2.58 (m, 4H), 1.70-1.39 (m, 4H).

### Example 307 (Isomer 1): (2R,6S)-4-(8-cyanoquinoxalin-5-yl)-N-[[(3R)-3-fluoropyrrolidin-3-yl]methyl]-6-(trifluoromethyl)morpholine-2-carboxamide & Example 308 (Isomer 2): (2S,6R)-4-(8-cyanoquinoxalin-5-yl)-N-[[(3R)-3-fluoropyrrolidin-3-yl]methyl]-6-(trifluoromethyl)morpholine-2-carboxamide

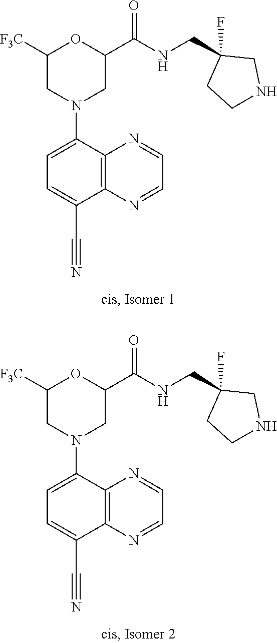 [SMILES: N#Cc1ccc(N2CC(C(=O)NC[C@]3(F)CCNC3)OC(C(F)(F)F)C2)c2nccnc12 ; N#Cc1ccc(N2CC(C(=O)NC[C@]3(F)CCNC3)OC(C(F)(F)F)C2)c2nccnc12]

The title compounds were prepared from cis-4-(8-cyanoquinoxalin-5-yl)-6-(trifluoromethyl)morpholine-2-carboxylic acid and tert-butyl (3R)-3-(aminomethyl)-3-fluoropyrrolidine-1-carboxylate. The two isomers were obtained by separation on chiral-HPLC under the following conditions: column, CHIRALPAK Cellulose-SB, 0.46×15 cm, 3 um; mobile phase, MtBE (with 0.1% DEA) in EtOH, 70% isocratic in 20 min; detector, UV 254 nm.

Isomer 1: MS: 453 [M+H]⁺. ¹H NMR (400 MHz, DMSO-d₆, ppm) δ 9.05 (d, J=1.8 Hz, 1H), 9.03 (d, J=1.8 Hz, 1H), 8.28 (d, J=8.3 Hz, 1H), 8.14-8.09 (m, 1H), 7.40 (d, J=8.4 Hz, 1H), 4.74-4.69 (m, 1H), 4.60-4.50 (m, 1H), 4.46-4.30 (m, 2H), 3.69-3.51 (m, 3H), 3.27-3.09 (m, 3H), 3.0-2.84 (m, 3H), 2.01-1.78 (m, 2H).

Isomer 2: MS: 453 [M+H]⁺. ¹H NMR (400 MHz, DMSO-d₆, ppm) δ 9.10 (d, J=1.8 Hz, 1H), 9.03 (d, J=1.8 Hz, 1H), 8.30 (d, J=8.3 Hz, 1H), 8.14-8.09 (m, 1H), 7.40 (d, J=8.4 Hz, 1H), 4.74-4.69 (m, 1H), 4.60-4.52 (m, 1H), 4.44-4.32 (m, 2H), 3.68-3.49 (m, 3H), 3.27-3.09 (m, 3H), 3.01-2.83 (m, 3H), 2.0-1.80 (m, 2H).

### Example 309 (Isomer 1): (2R,6S)-4-(8-cyanoquinoxalin-5-yl)-N-[[(3S)-3-fluoropyrrolidin-3-yl]methyl]-6-(trifluoromethyl)morpholine-2-carboxamide & Example 310 (Isomer 2): (2S,6R)-4-(8-cyanoquinoxalin-5-yl)-N-[[(3S)-3-fluoropyrrolidin-3-yl]methyl]-6-(trifluoromethyl)morpholine-2-carboxamide

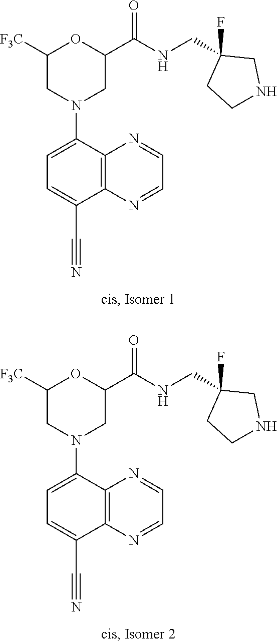 [SMILES: N#Cc1ccc(N2CC(C(=O)NC[C@@]3(F)CCNC3)OC(C(F)(F)F)C2)c2nccnc12 ; N#Cc1ccc(N2CC(C(=O)NC[C@@]3(F)CCNC3)OC(C(F)(F)F)C2)c2nccnc12]

The title compounds were prepared from cis-4-(8-cyanoquinoxalin-5-yl)-6-(trifluoromethyl)morpholine-2-carboxylic acid and tert-butyl (3S)-3-(aminomethyl)-3-fluoropyrrolidine-1-carboxylate. The two isomers were obtained by separation on chiral-HPLC under the following conditions: column, CHIRALPAK IA, 0.46×15 cm, 3 um; mobile phase, MtBE (with 0.1% DEA) in EtOH, 50% isocratic in 20 min; detector, UV 254 nm.

Isomer 1: MS: 453 [M+H]⁺. ¹H NMR (300 MHz, DMSO-d₆, ppm) δ 9.98 (br s, 1H), 9.73 (br s, 1H), 9.11-9.03 (m, 1.8 Hz, 2H), 8.40 (t, J=6.3 Hz, 1H), 8.27 (d, J=8.3 Hz, 1H), 7.40 (d, J=8.4 Hz, 1H), 4.80-4.51 (m, 2H), 4.49-4.20 (m, 2H), 3.72-3.56 (m, 2H), 3.41-3.07 (m, 6H), 2.24-2.02 (m, 2H).

Isomer 2: MS: 453 [M+H]⁺. ¹H NMR (300 MHz, DMSO-d₆, ppm) δ 9.05 (m, J=21.1, 1.6 Hz, 2H), 8.28 (m, J=8.3, 1.8 Hz, 1H), 8.06 (s, 1H), 7.42-7.33 (m, 1H), 4.69 (s, 1H), 4.55 (d, J=10.7 Hz, 1H), 4.36 (m, J=13.4 Hz, 2H), 3.65-3.41 (m, 3H), 3.16 (m, J=24.0, 11.7 Hz, 3H), 2.96-2.73 (m, 3H), 2.02-1.70 (m, 2H).

### Example 311 (Isomer 1): 8-[(2R,6S)-2-[(7S)-7-amino-5-azaspiro[2.4]heptane-5-carbonyl]-6-(trifluoromethyl)morpholin-4-yl]quinoxaline-5-carbonitrile & Example 312 (Isomer 2): 8-[(2S,6R)-2-[(7S)-7-amino-5-azaspiro[2.4]heptane-5-carbonyl]-6-(trifluoromethyl)morpholin-4-yl]quinoxaline-5-carbonitrile

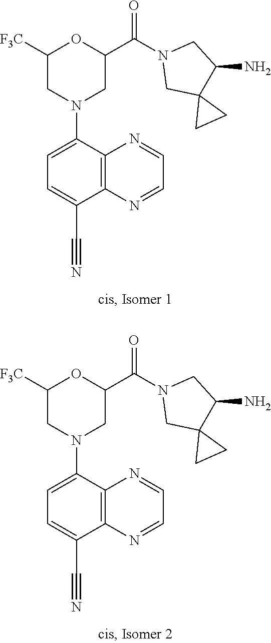 [SMILES: N#Cc1ccc(N2CC(C(=O)N3C[C@@H](N)C4(CC4)C3)OC(C(F)(F)F)C2)c2nccnc12 ; N#Cc1ccc(N2CC(C(=O)N3C[C@@H](N)C4(CC4)C3)OC(C(F)(F)F)C2)c2nccnc12]

The title compounds were prepared from cis-4-(8-cyanoquinoxalin-5-yl)-6-(trifluoromethyl)morpholine-2-carboxylic acid and tert-butyl N-[(7S)-5-azaspiro[2.4]heptan-7-yl]carbamate. The two isomers were obtained by separation on chiral-HPLC under the following conditions: column, CHIRALPAK ID-3, 0.46×5 cm, 3 um; mobile phase, MtBE (with 0.1% DEA) in MeOH, 80% isocratic in 20 min; detector, UV 254 nm.

Isomer 1: MS: 447 [M+H]⁺. ¹H NMR (400 MHz, DMSO-d₆, ppm) δ 9.12-9.07 (m, 1H), 9.05-8.97 (m, 1H), 8.32-8.26 (m, 1H), 7.43-7.36 (m, 1H), 4.80-4.66 (m, 1H), 4.47-4.26 (m, 2H), 3.95-3.93 (m, 0.5H), 3.78-3.75 (m, 0.5H), 3.56-3.52 (m, 1H), 3.39-3.33 (m, 1.5H), 3.27-3.12 (m, 3H), 3.09-3.01 (m, 0.5H), 1.66-1.61 (m, 1H), 0.82-0.74 (m, 1H), 0.67-0.33 (m, 3H).

Isomer 2: MS: 447 [M+H]⁺. ¹H NMR (400 MHz, DMSO-d₆, ppm) δ 9.20-9.15 (m, 1H), 9.05-8.97 (m, 1H), 8.32-8.26 (m, 1H), 7.43-7.36 (m, 1H), 4.80-4.66 (m, 1H), 4.47-4.26 (m, 2H), 3.95-3.93 (m, 0.5H), 3.82-3.35 (m, 3H), 3.29-3.15 (m, 3H), 3.11-3.01 (m, 0.5H), 1.65-1.58 (m, 1H), 0.80-0.72 (m, 1H), 0.66-0.30 (m, 3H).

### Example 313 (Isomer 1): (2S,6R)-4-(8-cyanoquinolin-5-yl)-N—(((S)-3-fluoropyrrolidin-3-yl)methyl)-6-(trifluoromethyl)morpholine-2-carboxamide & Example 314 (Isomer 2): (2R,6S)-4-(8-cyanoquinolin-5-yl)-N—(((S)-3-fluoropyrrolidin-3-yl)methyl)-6-(trifluoromethyl)morpholine-2-carboxamide

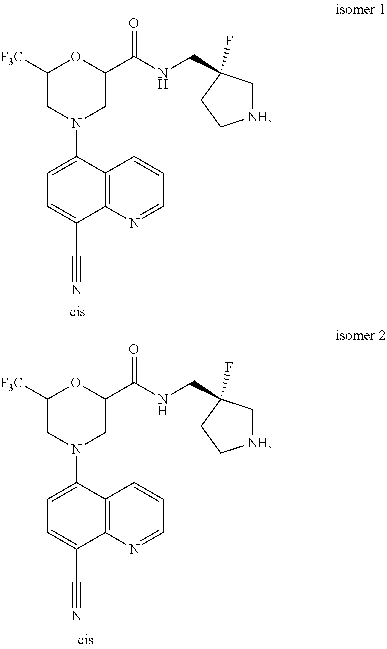 [SMILES: N#Cc1ccc(N2CC(C(=O)NC[C@]3(F)CCNC3)OC(C(F)(F)F)C2)c2cccnc12 ; N#Cc1ccc(N2CC(C(=O)NC[C@]3(F)CCNC3)OC(C(F)(F)F)C2)c2cccnc12]

The title compounds were prepared from cis-4-(8-cyanoquinolin-5-yl)-6-(trifluoromethyl)morpholine-2-carboxylic acid and tert-butyl (3R)-3-(aminomethyl)-3-fluoropyrrolidine-1-carboxylate. The two isomers were obtained by separation on chiral-HPLC under the following conditions: column, CHIRALPAK ID-3, 0.46×10 cm, 3 um; mobile phase, hexane (with 0.1% DEA) in EtOH, 50% isocratic in 25 min; detector, UV 220 nm.

Isomer 1: MS: 452 [M+H]⁺. ¹H NMR (300 MHz, Methanol-d₄, ppm) δ 9.06-8.98 (m, 1H), 8.72 (dd, J=8.6, 1.7 Hz, 1H), 8.19 (d, J=8.0 Hz, 1H), 7.70 (dd, J=8.6, 4.2 Hz, 1H), 7.38 (d, J=8.0 Hz, 1H), 4.80-4.68 (m, 2H), 3.80-3.51 (m, 4H), 3.24-2.87 (m, 6H), 2.18-1.83 (m, 2H). Isomer 2: MS: 452 [M+H]⁺. ¹H NMR (300 MHz, Methanol-d₄, ppm) δ 9.02 (m, J=4.3, 1.7 Hz, 1H), 8.72 (m, J=8.6, 1.7 Hz, 1H), 8.19 (d, J=8.0 Hz, 1H), 7.70 (m, J=8.6, 4.2 Hz, 1H), 7.38 (d, J=8.0 Hz, 1H), 4.80-4.63 (m, 2H), 3.84-3.63 (m, 3H), 3.57 (d, J=11.7 Hz, 1H), 3.23-2.78 (m, 6H), 2.0 (m, 2H).

### Example 315 (Isomer 1): (2R,6S)-4-(8-cyanoquinolin-5-yl)-N-[(4-fluoro-1-methylpiperidin-4-yl)methyl]-6-(trifluoromethyl)morpholine-2-carboxamide & Example 316 (Isomer 2): (2S,6S)-4-(8-cyanoquinolin-5-yl)-N-[(4-fluoro-1-methylpiperidin-4-yl)methyl]-6-(trifluoromethyl)morpholine-2-carboxamide

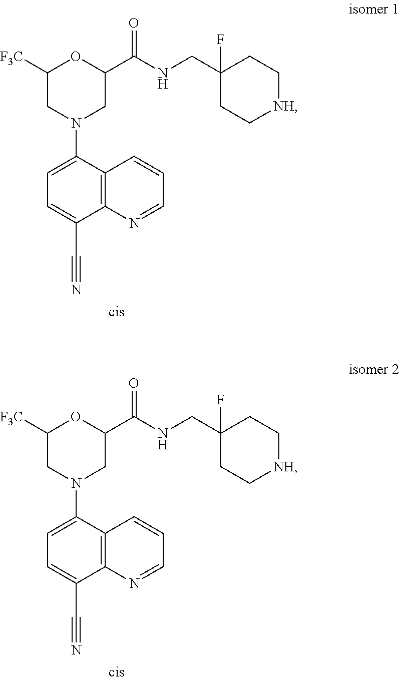 [SMILES: N#Cc1ccc(N2CC(C(=O)NCC3(F)CCNCC3)OC(C(F)(F)F)C2)c2cccnc12 ; N#Cc1ccc(N2CC(C(=O)NCC3(F)CCNCC3)OC(C(F)(F)F)C2)c2cccnc12]

The title compounds were prepared from cis-4-(8-cyanoquinolin-5-yl)-6-(trifluoromethyl)morpholine-2-carboxylic acid and tert-butyl 4-(aminomethyl)-4-fluoropiperidine-1-carboxylate. The two isomers were obtained by separation on chiral-HPLC under the following conditions: column, CHIRALPAK UH, 0.46×15 cm, 3 um; mobile phase, MeOH (with 0.1% DEA) in 25 min; detector, UV 220 nm.

Isomer 1: MS: 466 [M+H]⁺. ¹H NMR (300 MHz, DMSO-d₆, ppm) δ 9.10-9.03 (m, 1H), 8.68 (d, J=8.6 Hz, 1H), 8.27 (d, J=7.9 Hz, 1H), 8.02-7.92 (m, 1H), 7.78-7.67 (m, 1H), 7.45-7.36 (m, 1H), 4.86-4.76 (m, 1H), 4.71-4.62 (m, 1H), 3.70-2.88 (m, 7H), 2.76-2.56 (m, 4H), 1.72-1.32 (m, 4H).

Isomer 2: MS: 466 [M+H]⁺. ¹H NMR (300 MHz, DMSO-d₆, ppm) δ 9.07 (d, J=4.2 Hz, 1H), 8.68 (d, J=8.6 Hz, 1H), 8.27 (d, J=7.9 Hz, 1H), 7.97 (m, J=6.2 Hz, 1H), 7.73 (m, J=8.6, 4.3 Hz, 1H), 7.40 (d, J=8.1 Hz, 1H), 4.81 (d, J=9.6 Hz, 1H), 4.67 (d, J=10.3 Hz, 1H), 3.52 (m, J=10.8 Hz, 2H), 3.35 (d, J=6.1 Hz, 2H), 3.29 (m, J=5.5 Hz, 1H), 3.17-3.01 (m, 1H), 2.96 (d, J=11.7 Hz, 1H), 2.65 (d, J=14.8 Hz, 4H), 1.49 (m, 4H).

### Example 317 (Isomer 1): (2R,6S)-4-(8-cyano-1,7-naphthyridin-5-yl)-N-[(4-fluoropiperidin-4-yl)methyl]-6-(trifluoromethyl)morpholine-2-carboxamide & Example 318 (Isomer 2): (2S,6R)-4-(8-cyano-1,7-naphthyridin-5-yl)-N-[(4-fluoropiperidin-4-yl)methyl]-6-(trifluoromethyl)morpholine-2-carboxamide

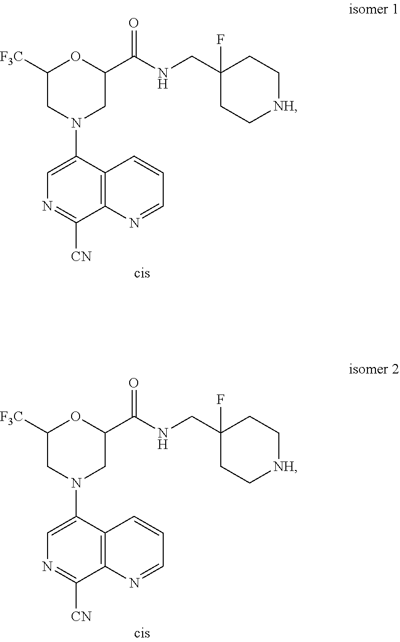 [SMILES: N#Cc1ncc(N2CC(C(=O)NCC3(F)CCNCC3)OC(C(F)(F)F)C2)c2cccnc12 ; N#Cc1ncc(N2CC(C(=O)NCC3(F)CCNCC3)OC(C(F)(F)F)C2)c2cccnc12]

The title compounds were prepared from cis-4-(8-cyano-1,7-naphthyridin-5-yl)-6-(trifluoromethyl)morpholine-2-carboxylic acid and tert-butyl 4-(aminomethyl)-4-fluoropiperidine-1-carboxylate. The two isomers were obtained by separation on chiral-HPLC under the following conditions: column, CHIRALPAK ID-3, 0.46×15 cm, 3 um; mobile phase, MtBE (with 0.1% DEA) in EtOH, 70% isocratic in 20 min; detector, UV 254 nm.

Isomer 1: MS: 467 [M+H]⁺. ¹H NMR (400 MHz, DMSO-d₆, ppm) δ 9.25-9.19 (m, 1H), 8.76-8.69 (m, 1H), 8.56 (s, 1H), 8.10-7.81 (m, 2H), 4.89-4.77 (m, 1H), 4.72-4.65 (m, 1H), 3.71-3.62 (m, 2H), 3.46-3.28 (m, 2H), 3.19-3.09 (m, 3H), 2.83-2.57 (m, 4H), 1.71-1.38 (m, 4H).

Isomer 2: MS: 467 [M+H]⁺. ¹H NMR (400 MHz, DMSO-d₆, ppm) δ 9.20-9.13 (m, 1H), 8.73-8.66 (m, 1H), 8.55 (s, 1H), 8.08-7.79 (m, 2H), 4.85-4.74 (m, 1H), 4.70-4.62 (m, 1H), 3.68-3.59 (m, 2H), 3.46-3.26 (m, 2H), 3.22-3.07 (m, 3H), 2.80-2.55 (m, 4H), 1.70-1.35 (m, 4H).

### Example 319 (Isomer 1): (2S,6R)-4-(8-cyano-1,7-naphthyridin-5-yl)-N-[[(3S)-3-fluoropyrrolidin-3-yl]methyl]-6-(trifluoromethyl)morpholine-2-carboxamide & Example 320 (Isomer 2): (2R,6S)-4-(8-cyano-1,7-naphthyridin-5-yl)-N-[[(3S)-3-fluoropyrrolidin-3-yl]methyl]-6-(trifluoromethyl)morpholine-2-carboxamide

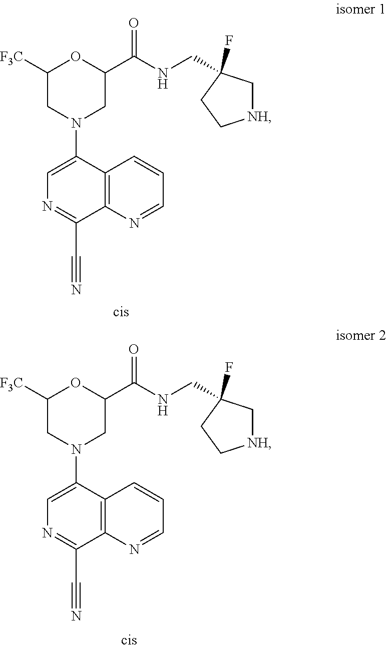 [SMILES: N#Cc1ncc(N2CC(C(=O)NC[C@@]3(F)CCNC3)OC(C(F)(F)F)C2)c2cccnc12 ; N#Cc1ncc(N2CC(C(=O)NC[C@@]3(F)CCNC3)OC(C(F)(F)F)C2)c2cccnc12]

The title compounds were prepared from cis-4-(8-cyano-1,7-naphthyridin-5-yl)-6-(trifluoromethyl)morpholine-2-carboxylic acid and tert-butyl (3S)-3-(aminomethyl)-3-fluoropyrrolidine-1-carboxylate. The two isomers were obtained by separation on chiral-HPLC under the following conditions: column, CHIRALPAK IC, 0.46×10 cm, 3 um; mobile phase, DCM (with 0.1% DEA) in MeOH, 50% isocratic in 25 min; detector, UV 220 nm.

Isomer 1: MS: 453 [M+H]⁺. ¹H NMR (400 MHz, DMSO-d₆, ppm) δ 9.25-9.19 (m, 1H), 8.73 (dd, J=8.7, 1.6 Hz, 1H), 8.56 (s, 1H), 8.13 (t, J=6.2 Hz, 1H), 7.95 (dd, J=8.7, 4.1 Hz, 1H), 4.88-4.79 (m, 1H), 4.71-4.64 (m, 1H), 3.71-3.63 (m, 2H), 3.60-3.47 (m, 2H), 3.30-3.22 (m, 2H), 3.20-3.09 (m, 1H), 2.95-2.72 (m, 4H), 1.92-1.70 (m, 2H).

Isomer 2: MS: 453 [M+H]⁺. ¹H NMR (400 MHz, DMSO-d₆, ppm) δ 9.20-9.16 (m, 1H), 8.69 (dd, J=8.8, 1.6 Hz, 1H), 8.59 (s, 1H), 8.11 (t, J=6.2 Hz, 1H), 7.95 (dd, J=8.8, 4.1 Hz, 1H), 4.90-4.79 (m, 1H), 4.68-4.59 (m, 1H), 3.75-3.67 (m, 2H), 3.58-3.45 (m, 2H), 3.29-3.09 (m, 3H), 2.99-2.70 (m, 4H), 1.88-1.65 (m, 2H).

### Example 321: (S)-2-{[(2R,6R)-4-(8-Cyano-quinoxalin-5-yl)-6-methyl-morpholine-2-carbonyl]-amino}-3-hydroxy-propionic acid

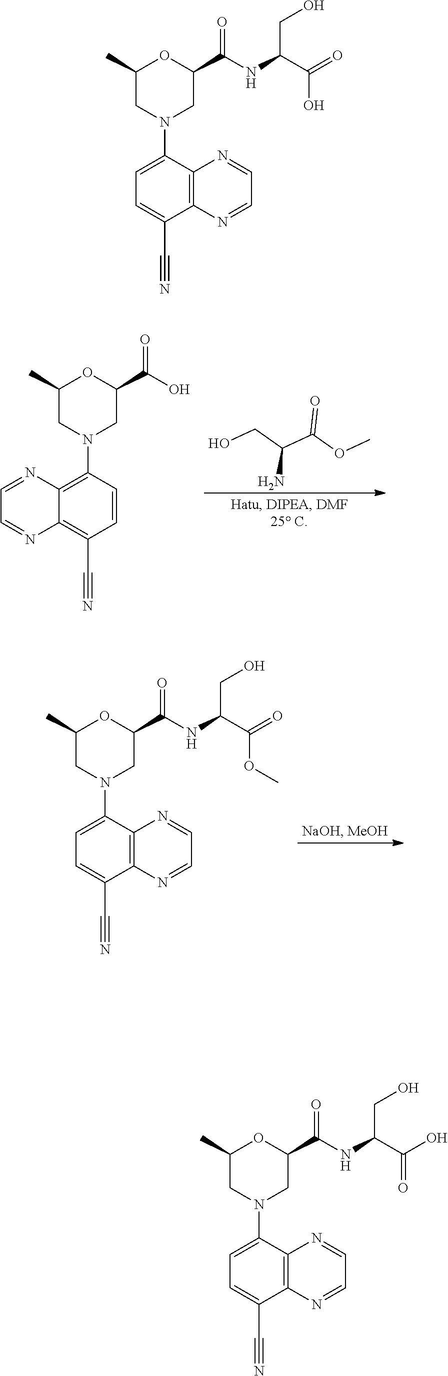

(S)-2-{[(2R,6R)-4-(8-Cyano-quinoxalin-5-yl)-6-methyl-morpholine-2-carbonyl]-amino}-3-hydroxy-propionic acid methyl ester: Into a 50 mL round-bottom flask, was placed (2R,6R)-4-(8-Cyano-quinoxalin-5-yl)-6-methyl-morpholine-2-carboxylic acid (70.0 mg; 0.23 mmol; 1.0 eq.) in DMF (2.0 ml). Hatu (107.07 mg; 0.28 mmol; 1.20 eq.) was added and the resulting solution stirred for 10 minutes at rt after which 1-serine methyl ester hydrochloride (43.81 mg; 0.28 mmol; 1.20 eq.) and DIPEA (0.12 ml; 0.70 mmol; 3.0 eq.) were respectively added. The resulting mixture was stirred at room temperature for 2 h. The volatils were evaporated and the residue dissolved in 4 mL of DMSO. The product was purified on reverse phase system using a gradient of 05-95% CH₃CN/H₂O (0.1% Ammonium Hydroxide) in 4 injections of 1 mL each. The desired fractions were evaporated to provide the title compound (49.0 mg; 52%) as a yellow gum. MS:400 [M+H]⁺.

(S)-2-{[(2R,6R)-4-(8-Cyano-quinoxalin-5-yl)-6-methyl-morpholine-2-carbonyl]-amino}-3-hydroxy-propionic acid: Into a 50 mL round-bottom flask, was placed (S)-2-{[(2R,6R)-4-(8-Cyano-quinoxalin-5-yl)-6-methyl-morpholine-2-carbonyl]-amino}-3-hydroxy-propionic acid methyl ester (200.0 mg; 0.50 mmol; 1.0 eq.) in MeOH (18.0 ml). Then NaOH (500.75 μl; 5.01 mmol; 10.0 eq.) was added and the resulting solution stirred for 30 minutes at 60° C. LC/MS showed the reaction was complete. The mixture was purified on waters reverse phase system using a gradient of 05-95% CH₃CN/H₂O (0.1% formic acid) in 6 injections of 3 mL each. The desired fractions were evaporated to give the title compound (144.0 mg; 75%) as a yellow solid. MS:386 [M+H]⁺. ¹H NMR (400 MHz, DMSO-d₆) δ 9.07 (d, J=1.4 Hz, 1H), 9.0 (d, J=1.6 Hz, 1H), 8.25 (d, J=8.3 Hz, 1H), 7.62 (d, J=7.7 Hz, 1H), 7.27 (d, J=8.4 Hz, 1H), 4.53 (d, J=12.2 Hz, 1H), 4.36 (dt, J=11.1, 3.2 Hz, 1H), 4.28 (dt, J=8.2, 4.1 Hz, 1H), 4.11 (d, J=12.4 Hz, 1H), 4.06-3.95 (m, 1H), 3.80 (dd, J=10.9, 4.3 Hz, 1H), 3.65 (td, J=10.2, 9.7, 3.9 Hz, 1H), 3.31 (s, 2H), 3.01-2.78 (m, 2H), 1.29 (d, J=6.2 Hz, 3H).

### Example 322 (Isomer 1): (2R,6R)-4-(8-Cyano-quinoxalin-5-yl)-6-methyl-morpholine-2-carboxylic acid ((S)-1-cyclopropylmethyl-pyrrolidin-3-yl)-amide & Example 323 (Isomer 2): (2R,6R)-4-(8-Cyano-quinoxalin-5-yl)-6-methyl-morpholine-2-carboxylic acid ((R)-cyclopropylmethyl-pyrrolidin-3-yl)-amide

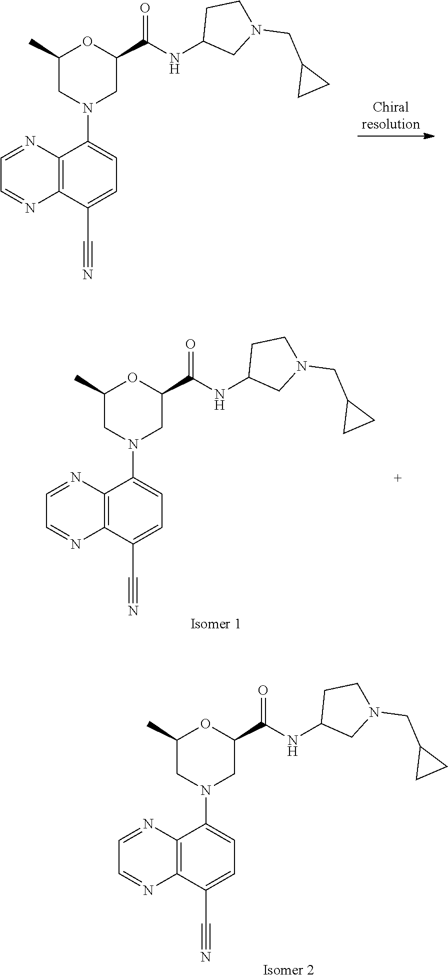 [SMILES: C[C@@H]1CN(c2ccc(C#N)c3nccnc23)C[C@H](C(=O)NC2CCN(CC3CC3)C2)O1 ; C[C@@H]1CN(c2ccc(C#N)c3nccnc23)C[C@H](C(=O)NC2CCN(CC3CC3)C2)O1 ; C[C@@H]1CN(c2ccc(C#N)c3nccnc23)C[C@H](C(=O)NC2CCN(CC3CC3)C2)O1]

The 2 isomers were obtained by separation of (2R,6R)-4-(8-Cyano-quinoxalin-5-yl)-6-methyl-morpholine-2-carboxylic acid (1-cyclopropylmethyl-pyrrolidin-3-yl)-amide on chiral prep-HPLC under the following conditions: column, AS-H, Prep SFC-P100; mobile phase, methanol+20 Mm NH₄OH, 40° C./80 bar, 100 g/min; detector, PDA.

Isomer 1: MS: 421 [M+H]⁺. ¹H NMR (400 MHz, Methanol-d₄) δ 8.97 (s, 1H), 8.92 (d, J=2.0 Hz, 1H), 8.13 (dd, J=8.3, 1.7 Hz, 1H), 7.33-7.13 (m, 1H), 4.61-4.47 (m, 2H), 4.42 (dt, J=10.8, 2.2 Hz, 1H), 4.20-3.99 (m, 2H), 3.08-2.66 (m, 5H), 2.53-2.42 (m, 1H), 2.39-2.25 (m, 3H), 1.73 (dt, J=13.7, 6.8 Hz, 1H), 1.37 (dd, J=6.2, 1.7 Hz, 3H), 0.94 (d, J=7.8 Hz, 1H), 0.64-0.47 (m, 2H), 0.28-0.11 (m, 2H).

Isomer 2: MS: 421 [M+H]⁺. ¹H NMR (400 MHz, Methanol-d₄) δ 8.97 (s, 1H), 8.92 (d, J=2.0 Hz, 1H), 8.13 (dd, J=8.3, 1.7 Hz, 1H), 7.33-7.13 (m, 1H), 4.61-4.47 (m, 2H), 4.42 (dt, J=10.8, 2.2 Hz, 1H), 4.20-3.99 (m, 2H), 3.08-2.60 (m, 5H), 2.50-2.41 (m, 1H), 2.40-2.25 (m, 3H), 1.73 (dt, J=13.7, 6.8 Hz, 1H), 1.37 (dd, J=6.2, 1.7 Hz, 3H), 0.94 (d, J=7.8 Hz, 1H), 0.64-0.47 (m, 2H), 0.28-0.11 (m, 2H).

### Example 324 (Isomer 1): (2R,6R)-4-(8-Cyano-quinoxalin-5-yl)-6-methyl-morpholine-2-carboxylic acid ((S)-4-methyl-morpholin-2-ylmethyl)-amide & Example 325 (Isomer 2): (2R,6R)-4-(8-Cyano-quinoxalin-5-yl)-6-methyl-morpholine-2-carboxylic acid ((R)-4-methyl-morpholin-2-ylmethyl)-amide

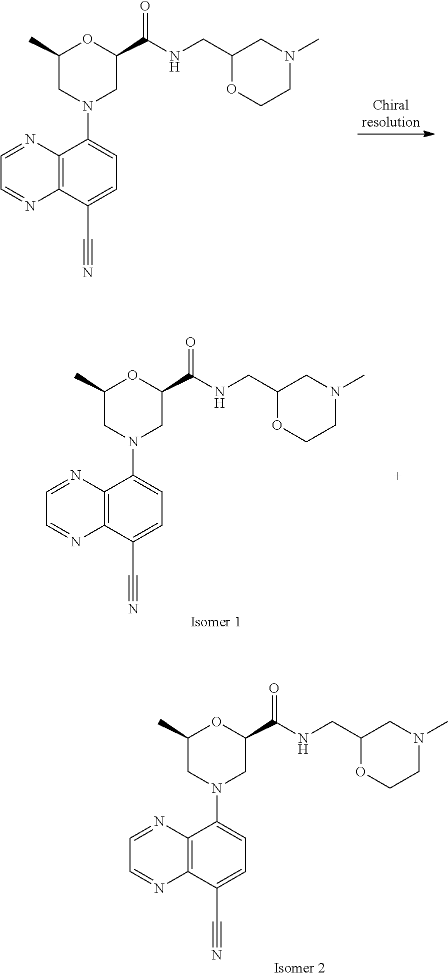 [SMILES: C[C@@H]1CN(c2ccc(C#N)c3nccnc23)C[C@H](C(=O)NCC2CN(C)CCO2)O1 ; C[C@@H]1CN(c2ccc(C#N)c3nccnc23)C[C@H](C(=O)NCC2CN(C)CCO2)O1 ; C[C@@H]1CN(c2ccc(C#N)c3nccnc23)C[C@H](C(=O)NCC2CN(C)CCO2)O1]

The 2 isomers were obtained by separation of (2R,6R)-4-(8-Cyano-quinoxalin-5-yl)-6-methyl-morpholine-2-carboxylic acid (4-methyl-morpholin-2-ylmethyl)-amide on chiral prep-HPLC under the following conditions: column, WHELKO-01, Prep SFC-P100; mobile phase, methanol+20 Mm NH₄OH, 40° C./80 bar, 100 g/min; detector, PDA.

Isomer 1: MS: 411 [M+H]⁺. ¹H NMR (400 MHz, Methanol-d₄) δ 9.01-8.95 (m, 1H), 8.92 (d, J=1.6 Hz, 1H), 8.13 (d, J=8.3 Hz, 1H), 7.24 (d, J=8.3 Hz, 1H), 4.57 (dt, J=12.2, 2.4 Hz, 1H), 4.44 (dd, J=10.8, 2.8 Hz, 1H), 4.21-4.04 (m, 2H), 3.91 (dd, J=11.7, 3.2 Hz, 1H), 3.65 (ddt, J=11.5, 7.8, 2.7 Hz, 2H), 3.42 (dd, J=13.8, 4.8 Hz, 1H), 3.35-3.26 (m, 1H), 2.96-2.77 (m, 3H), 2.70 (t, J=11.8 Hz, 1H), 2.32 (s, 3H), 2.18 (td, J=11.6, 3.4 Hz, 1H), 1.91 (t, J=10.9 Hz, 1H), 1.36 (d, J=6.1 Hz, 3H).

Isomer 2: MS: 411 [M+H]⁺. ¹H NMR (400 MHz, Methanol-d₄) δ 8.97 (d, J=1.5 Hz, 1H), 8.95-8.89 (m, 1H), 8.14 (d, J=8.3 Hz, 1H), 7.26 (d, J=8.3 Hz, 1H), 4.57 (dt, J=12.2, 2.4 Hz, 1H), 4.45 (dd, J=10.8, 2.8 Hz, 1H), 4.21-4.04 (m, 2H), 3.92 (dd, J=11.8, 3.2 Hz, 1H), 3.70-3.60 (m, 2H), 3.40 (dd, J=13.7, 4.7 Hz, 1H), 3.32 (dd, J=13.6, 6.9 Hz, 1H), 2.97-2.78 (m, 3H), 2.72 (d, J=11.8 Hz, 1H), 2.33 (s, 3H), 2.19 (td, J=11.6, 3.4 Hz, 1H), 1.92 (t, J=10.9 Hz, 1H), 1.35 (s, 3H).

### Example 326 (Isomer 1): (2R,6R)-4-(8-Cyano-quinoxalin-5-yl)-6-methyl-morpholine-2-carboxylic acid ((R)-2-hydroxy-3-methoxy-propyl)-amide & Example 327 (Isomer 2): (2R,6R)-4-(8-Cyano-quinoxalin-5-yl)-6-methyl-morpholine-2-carboxylic acid ((S)-2-hydroxy-3-methoxy-propyl)-amide

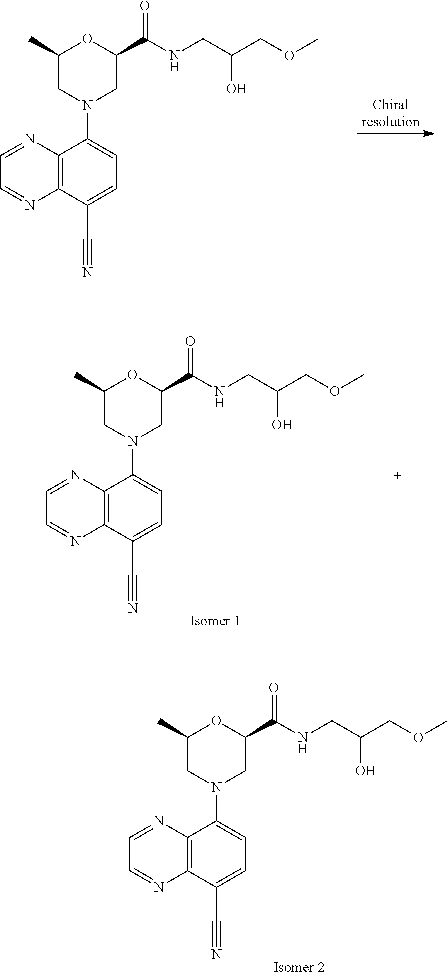 [SMILES: COCC(O)CNC(=O)[C@H]1CN(c2ccc(C#N)c3nccnc23)C[C@@H](C)O1 ; COCC(O)CNC(=O)[C@H]1CN(c2ccc(C#N)c3nccnc23)C[C@@H](C)O1 ; COCC(O)CNC(=O)[C@H]1CN(c2ccc(C#N)c3nccnc23)C[C@@H](C)O1]

The 2 isomers were obtained by separation of (2R,6R)-4-(8-cyanoquinoxalin-5-yl)-N-(2-hydroxy-3-methoxypropyl)-6-methylmorpholine-2-carboxamide on chiral prep-HPLC under the following conditions: column, IC-H, Prep SFC-P100; mobile phase, methanol+20 Mm NH₄OH, 40° C./80 bar, 100 g/min; detector, PDA.

Isomer 1: MS: 386 [M+H]⁺. H NMR (400 MHz, Methanol-d₄) δ 8.97 (s, 1H), 8.93 (s, 1H), 8.14 (d, J=8.2 Hz, 1H), 7.25 (d, J=8.3 Hz, 1H), 4.57 (d, J=12.3 Hz, 1H), 4.47-4.41 (m, 1H), 4.13 (dd, J=20.4, 8.5 Hz, 2H), 3.86 (q, J=5.0, 4.5 Hz, 1H), 3.53-3.36 (m, 6H), 3.31-3.22 (m, 1H), 2.92 (td, J=12.2, 11.6, 2.5 Hz, 1H), 2.83 (t, J=11.2 Hz, 1H), 1.36 (d, J=6.1 Hz, 3H).

Isomer 2: MS: 386 [M+H]⁺. H NMR (400 MHz, Methanol-d₄) δ 8.97 (s, 1H), 8.93 (s, 1H), 8.14 (d, J=8.2 Hz, 1H), 7.25 (d, J=8.3 Hz, 1H), 4.57 (d, J=12.3 Hz, 1H), 4.47-4.41 (m, 1H), 4.13 (dd, J=20.4, 8.5 Hz, 2H), 3.86 (q, J=5.0, 4.5 Hz, 1H), 3.53-3.36 (m, 6H), 3.31-3.22 (m, 1H), 2.92 (td, J=12.2, 11.6, 2.5 Hz, 1H), 2.83 (t, J=11.2 Hz, 1H), 1.36 (d, J=6.1 Hz, 3H).

### Example 328: 5-[(2R,6S)-2-Methyl-6-(4-pyrrolidin-1-yl-piperidin-1-ylmethyl)-morpholin-4-yl]-8-trifluoromethyl-quinoline

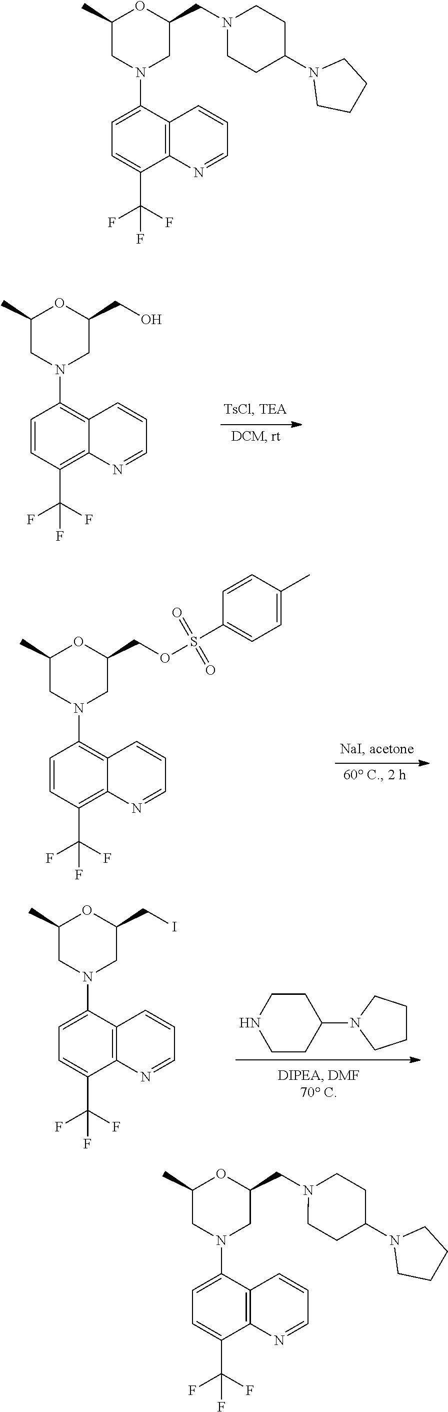

Toluene-4-sulfonic acid (2R,6R)-6-methyl-4-(8-trifluoromethyl-quinolin-5-yl)-morpholin-2-ylmethyl ester: Into a 20 mL schlenck reactor, was placed [(2R,6R)-6-Methyl-4-(8-trifluoromethyl-quinolin-5-yl)-morpholin-2-yl]-methanol (240.0 mg; 0.74 mmol; 1.0 eq.), DCM (10.0 ml), 4-methylbenzene-1-sulfonyl chloride (280.44 mg; 1.47 mmol; 2.0 eq.). This was followed by the addition of TEA (205.02 μl; 1.47 mmol; 2.0 eq.) with stirring at 20° C. The resulting solution was stirred for 3 h at 20° C. The reaction was then quenched by the addition of 20 mL of water. The resulting solution was extracted with 2×20 mL of DCM and the organic layers were combined and dried over anhydrous sodium sulfate and concentrated under vacuum. The residue was purified by chromatography on a Biotage (PuriFlash Column, 15μ Si HP, 12 g); hexane/ethyl acetate, gradient from 08-20% to 20-80% for 15 minutes to give the title compound (247.0 mg; 70%) as a colorless solid. MS: 481 [M+H]⁺.

5-((2R,6R)-2-Iodomethyl-6-methyl-morpholin-4-yl)-8-trifluoromethyl-quinoline: Into a 25 mL vial was placed Toluene-4-sulfonic acid (2R,6R)-6-methyl-4-(8-trifluoromethyl-quinolin-5-yl)-morpholin-2-ylmethyl ester (240.0 mg; 0.50 mmol; 1.0 eq.), sodium iodide (374.34 mg; 2.50 mmol; 5.0 eq.) and acetone (5.0 ml). The resulting solution was stirred for 16 h at 70° C. The solvent was evaporated and the residue extracted with ethyl acetate (50 mL) and 50 mL aqueous NaHSO₃ (5%) solution. The organic phase was dried with Na₂SO₄ and concentrated to give the title compound (211.0 mg; 97%) as a yellow solid. MS: 437 [M+H]⁺.

5-[(2R,6S)-2-Methyl-6-(4-pyrrolidin-1-yl-piperidin-1-ylmethyl)-morpholin-4-yl]-8-trifluoromethyl-quinoline: Into a 25-mL vial was placed 5-((2R,6R)-2-Iodomethyl-6-methyl-morpholin-4-yl)-8-trifluoromethyl-quinoline (30.0 mg; 0.07 mmol; 1.0 eq.), 4-(1-pyrrolidinyl)piperidine (21.22 mg; 0.14 mmol; 2.0 eq.), DMF (1.50 ml), TEA (29.91 μl; 0.22 mmol; 3.13 eq.). The resulting solution was heated at 80° C. for 2 h. The reaction mixture was filtered through celite, concentrated under reduced pressure, and dissolved in DCM (2 mL). The solution was absorbed on a PuriFlash 4 g column and purified by chromatography (DCM-MeOH, gradient 98-2% to 90-10% for 18 minutes. The pure fractions were concentrated under reduced pressure to give the title compound (20.80 mg; 65%) as an off-white solid. MS: 463 [M+H]⁺. ¹H NMR (400 MHz, Methanol-d₄) δ 8.96 (dd, J=4.2, 1.8 Hz, 1H), 8.69 (dd, J=8.6, 1.8 Hz, 1H), 8.06 (d, J=8.0 Hz, 1H), 7.63 (dd, J=8.6, 4.2 Hz, 1H), 7.26 (d, J=8.0 Hz, 1H), 4.21 (dddd, J=9.7, 7.0, 4.8, 2.2 Hz, 1H), 4.10 (dtt, J=12.4, 6.2, 3.1 Hz, 1H), 3.47-3.26 (m, 8H), 3.24-3.15 (m, 2H), 2.72-2.56 (m, 4H), 2.35 (q, J=13.3 Hz, 2H), 2.23-2.14 (m, 2H), 2.13-2.01 (m, 4H), 1.79 (qdd, J=12.0, 6.2, 4.2 Hz, 2H), 1.37-1.29 (m, 1H), 1.27 (d, J=6.2 Hz, 3H).

The following compounds were synthesized in an analogous manner:

### Example 329: 5-[(2R,6S)-2-Methyl-6-(4-morpholin-4-yl-piperidin-1-ylmethyl)-morpholin-4-yl]-8-trifluoromethyl-quinoline

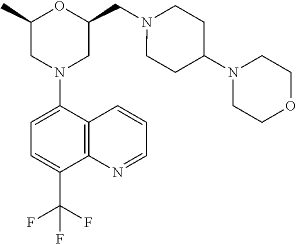 [SMILES: C[C@@H]1CN(c2ccc(C(F)(F)F)c3ncccc23)C[C@H](CN2CCC(N3CCOCC3)CC2)O1]

The title compound was prepared from 5-((2R,6R)-2-Iodomethyl-6-methyl-morpholin-4-yl)-8-trifluoromethyl-quinoline and 4-(piperidin-4-yl)morpholine. MS: 479 [M+H]⁺. ¹H NMR (400 MHz, Chloroform-d) δ 9.0 (dd, J=4.3, 1.6 Hz, 1H), 8.74 (dd, J=8.7, 1.7 Hz, 1H), 8.10 (d, J=8.0 Hz, 1H), 7.67 (dd, J=8.6, 4.2 Hz, 1H), 7.30 (d, J=8.0 Hz, 1H), 4.44-4.31 (m, 1H), 4.17 (ddt, J=11.1, 6.8, 3.5 Hz, 1H), 3.77 (q, J=5.8, 5.2 Hz, 4H), 3.55-3.40 (m, 3H), 2.96 (d, J=6.0 Hz, 2H), 2.76-2.65 (m, 8H), 2.51 (tt, J=11.0, 3.7 Hz, 1H), 2.11 (dqd, J=13.0, 6.3, 3.7, 3.1 Hz, 2H), 1.80 (q, J=12.5 Hz, 2H), 1.35 (dd, J=20.1, 6.7 Hz, 4H).

### Example 330: 5-[(2R,6S)-2-Methyl-6-(4-methyl-piperazin-1-ylmethyl)-morpholin-4-yl]-8-trifluoromethyl-quinoline

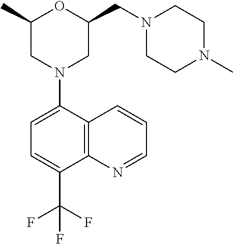

The title compound was prepared from 5-((2R,6R)-2-Iodomethyl-6-methyl-morpholin-4-yl)-8-trifluoromethyl-quinoline and 1-Methyl-piperazine. MS: 409 [M+H]⁺. ¹H NMR (400 MHz, Methanol-d₄) δ 8.96 (dd, J=4.2, 1.7 Hz, 1H), 8.69 (dd, J=8.6, 1.7 Hz, 1H), 8.06 (d, J=8.0 Hz, 1H), 7.63 (dd, J=8.6, 4.2 Hz, 1H), 7.27 (d, J=8.0 Hz, 1H), 4.21 (t, J=8.3 Hz, 1H), 4.11 (ddd, J=10.1, 6.2, 2.3 Hz, 1H), 3.51-2.93 (m, 10H), 2.83 (s, 3H), 2.79-2.58 (m, 4H), 2.61 (s, OH), 1.27 (d, J=6.3 Hz, 3H).

### Example 331: 2-{1-[(2S,6R)-6-Methyl-4-(8-trifluoromethyl-quinolin-5-yl)-morpholin-2-ylmethyl]-pyrrolidin-3-yl}-propan-2-ol

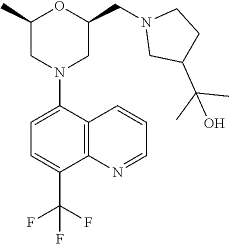 [SMILES: C[C@@H]1CN(c2ccc(C(F)(F)F)c3ncccc23)C[C@H](CN2CCC(C(C)(C)O)C2)O1]

The title compound was prepared from 5-((2R,6R)-2-Iodomethyl-6-methyl-morpholin-4-yl)-8-trifluoromethyl-quinoline and 2-(pyrrolidin-3-yl)propan-2-ol. MS: 438 [M+H]⁺. ¹H NMR (400 MHz, Methanol-d₄) δ 8.97 (d, J=5.4 Hz, 1H), 8.73 (d, J=8.6 Hz, 1H), 8.08 (d, J=8.0 Hz, 1H), 7.65 (dd, J=8.6, 4.2 Hz, 1H), 7.29 (d, J=8.0 Hz, 1H), 4.43 (t, J=9.8 Hz, 1H), 4.29-4.12 (m, 1H), 3.61-3.36 (m, 8H), 2.73 (td, J=11.2, 4.5 Hz, 2H), 2.56 (q, J=8.6 Hz, 1H), 2.15 (q, J=8.2, 7.6 Hz, 2H), 1.35-1.31 (m, 3H), 1.29-1.25 (m, 6H).

### Example 332: N-{[(2S,6R)-4-(8-cyano-1,7-naphthyridin-5-yl)-6-methylmorpholin-2-yl]methyl}-3-fluoropyrrolidine-3-carboxamide

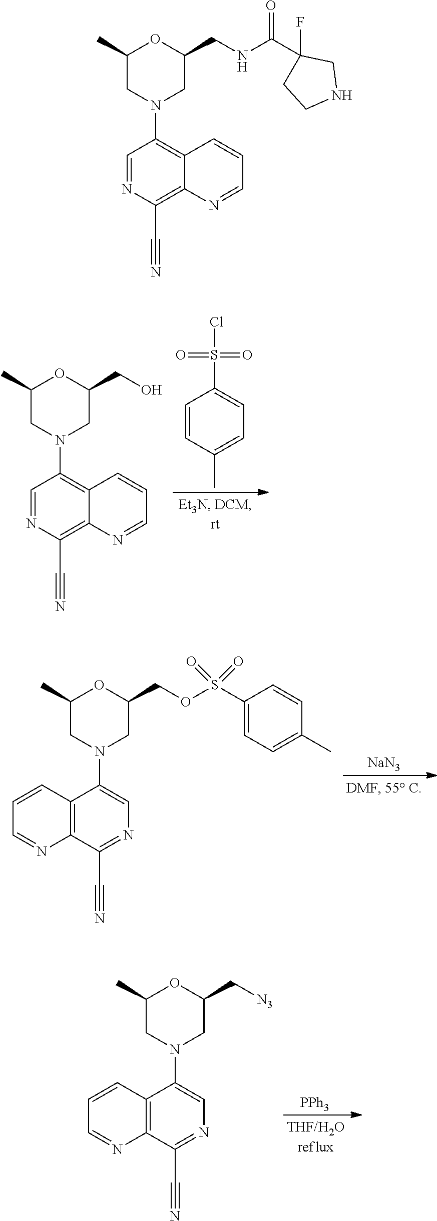

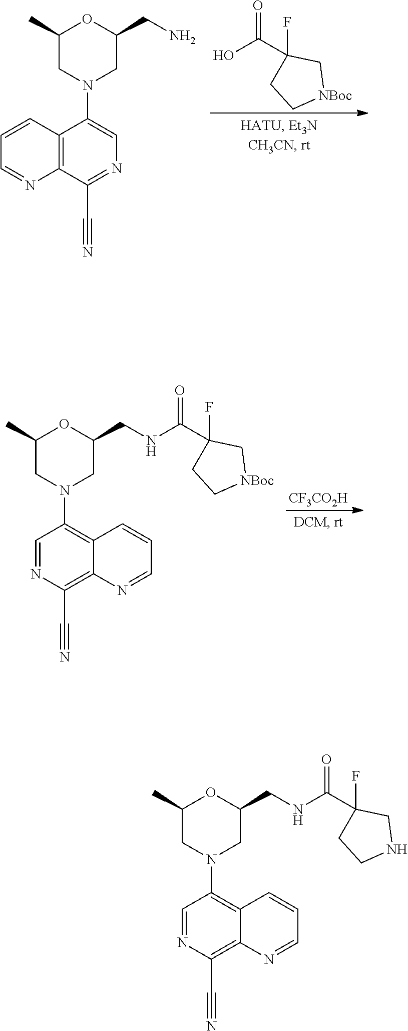 [SMILES: C[C@@H]1CN(c2cnc(C#N)c3ncccc23)C[C@H](CN)O1 ; CC(C)(C)OC(=O)N1CCC(F)(C(=O)O)C1 ; C[C@@H]1CN(c2cnc(C#N)c3ncccc23)C[C@H](CNC(=O)C2(F)CCN(C(=O)OC(C)(C)C)C2)O1 ; "CF3CO2H" ; C[C@@H]1CN(c2cnc(C#N)c3ncccc23)C[C@H](CNC(=O)C2(F)CCNC2)O1]

[(2R,6R)-4-(8-cyano-1,7-naphthyridin-5-yl)-6-methylmorpholin-2-yl]methyl 4-methylbenzene-1-sulfonate: To a stirred solution of 5-[(2R,6R)-2-(hydroxymethyl)-6-methylmorpholin-4-yl]-1,7-naphthyridine-8-carbonitrile (0.76 g; 2.66 mmol; 1.0 eq.) in DCM (2.0 ml) at room temperature was added p-toluenesulfonyl chloride (0.61 g; 3.19 mmol; 1.20 eq.), followed by TEA (0.74 ml; 5.31 mmol; 2.0 eq.). The mixture was stirred at room temp for 2 hours. The mixture was quenched by adding water, extracted with EtOAc. The organic layer was dried over Na₂SO₄ and concentrated to give the title compound as a pale yellow solid (1200 mg; crude). MS:439 [M+H]⁺.

5-[(2R,6R)-2-(azidomethyl)-6-methylmorpholin-4-yl]-1,7-naphthyridine-8-carbonitrile: To a stirred solution of [(2R,6R)-4-(8-cyano-1,7-naphthyridin-5-yl)-6-methylmorpholin-2-yl]methyl 4-methylbenzene-1-sulfonate (2143.78 mg; 4.40 mmol; 1.0 eq.) in DMF (2.0 ml) at room temperature was added sodium azide (429.07 mg; 6.60 mmol; 1.50 eq.). The mixture was stirred at 55° C. for 2 hours. LCMS showed no starting material left. It was evaporated to give the title compound (1360 mg; crude). MS:310 [M+H]⁺.

5-[(2S,6R)-2-(aminomethyl)-6-methylmorpholin-4-yl]-1,7-naphthyridine-8-carbonitrile: To a stirred solution of 5-[(2R,6R)-2-(azidomethyl)-6-methylmorpholin-4-yl]-1,7-naphthyridine-8-carbonitrile (1333.20 mg; 4.31 mmol; 1.0 eq.) and triphenylphosphine (1690.0, 6.4 mmol, 1.5 eq) in THF (20.0 ml) was added H₂O at room temperature. The mixture was stirred at reflux for 4 hours. The reaction mixture was cooled to room temperature, diluted by adding water, extracted with EtOAc. The organic layer was concentrated to give the title compound (2300 mg; crude). MS:284 [M+H]⁺.

tert-butyl (3R)-3-({[(2S,6R)-4-(8-cyano-1,7-naphthyridin-5-yl)-6-methylmorpholin-2-yl]methyl}carbamoyl)-3-fluoropyrrolidine-1-carboxylate: Into a 50 mL round-bottom flask, was placed 5-[(2S,6R)-2-(aminomethyl)-6-methylmorpholin-4-yl]-1,7-naphthyridine-8-carbonitrile (100.0 mg; 0.302 mmol; 1.0 eq.) in ACN (2.0 ml). 1-[(tert-butoxy)carbonyl]-3-fluoropyrrolidine-3-carboxylic acid (105.1 mg; 0.453 mmol; 1.50 eq.), Hatu (172.1 mg; 0.453 mmol; 1.50 eq.) and DIPEA (157.7 μl; 0.905 mmol; 3.0 eq.) were respectively added. The resulting mixture was stirred at room temperature for 2 hours. LCMS showed the reaction was complete. The reaction mixture was filtered through celite and concentrated under vacuum. The residue was purified by chromatography on a Biotage (PuriFlash Column, 15μ Si HP, 10 g) with ethyl acetate/petroleum ether (10:100 to 50:50) for 18 minutes to yield the title compound. MS:499 [M+H]⁺.

N-{[(2S,6R)-4-(8-cyano-1,7-naphthyridin-5-yl)-6-methylmorpholin-2-yl]methyl}-3-fluoropyrrolidine-3-carboxamide: To a stirred solution of tert-butyl (3R)-3-({[(2S,6R)-4-(8-cyano-1,7-naphthyridin-5-yl)-6-methylmorpholin-2-yl]methyl}carbamoyl)-3-fluoropyrrolidine-1-carboxylate (141.66 mg; 0.40 mmol; 1.0 eq.) in DCM (2 ml) at room temperature was added trifluoro acetic acid (0.5 ml). The resulting mixture was stirred at room temperature for 2 h. The solvent was removed. The residue was purified by reverse phase column to give the title compound (24 mg, 20%). MS: 399 [M+H]⁺. ¹H NMR (400 MHz, CDCl₃) δ 9.18 (dd, J=4.2, 1.6 Hz, 1H), 8.39 (dd, J=8.7, 1.6 Hz, 1H), 8.30 (s, 1H), 7.70 (dd, J=8.6, 4.1 Hz, 1H), 6.93 (s, 1H), 4.06-4.0 (m, 2H), 3.74-3.66 (m, 1H), 3.43-3.28 (m, 4H), 3.28-3.08 (m, 3H), 2.86-2.74 (m, 2H), 2.46-2.29 (m, 1H), 2.22-2.03 (m, 1H), 1.31 (d, J=6.3 Hz, 3H).

The following compounds were synthesized in an analogous manner.

### Example 333: N-{[(2S,6R)-4-(8-cyano-1,7-naphthyridin-5-yl)-6-methylmorpholin-2-yl]methyl}-3-fluoropiperidine-3-carboxamide

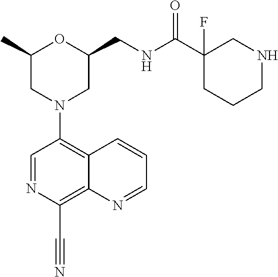 [SMILES: C[C@@H]1CN(c2cnc(C#N)c3ncccc23)C[C@H](CNC(=O)C2(F)CCCNC2)O1]

The title compound was prepared from 5-[(2S,6R)-2-(aminomethyl)-6-methylmorpholin-4-yl]-1,7-naphthyridine-8-carbonitrile and 1-[(tert-butoxy)carbonyl]-3-fluoropiperidine-3-carboxylic acid. MS: 399 [M+H]⁺. ¹H NMR (400 MHz, CDCl₃) δ 9.17 (d, J=4.6 Hz, 1H), 8.43-8.35 (m, 1H), 8.29 (d, J=3.4 Hz, 1H), 7.69 (dd, J=8.5, 4.2 Hz, 1H), 6.80 (d, J=81.3 Hz, 1H), 4.06-3.97 (m, 2H), 3.68-3.55 (m, 1H), 3.41-2.95 (m, 7H), 2.86-2.68 (m, 3H), 2.04-1.63 (m, 3H), 1.30 (t, J=5.3 Hz, 3H).

### Example 334 (Isomer 1): (3R)—N-{[(2S,6R)-4-(8-cyano-1,7-naphthyridin-5-yl)-6-methylmorpholin-2-yl]methyl}-3-fluoro-1-methylpyrrolidine-3-carboxamide & Example 335 (Isomer 2): (3S)—N-{[(2S,6R)-4-(8-cyano-1,7-naphthyridin-5-yl)-6-methylmorpholin-2-yl]methyl}-3-fluoro-1-methylpyrrolidine-3-carboxamide

The title compounds were made from 5-[(2S,6R)-2-(aminomethyl)-6-methylmorpholin-4-yl]-1,7-naphthyridine-8-carbonitrile and 3-fluoro-1-methylpyrrolidine-3-carboxylic acid. The two isomers were obtained from the SFC chiral separation. The SFC conditions are column, IG-H, Prep SFC-P100; mobile phase, methanol+20 mM NH₄OH, 45° C./80 bar, 100 g/min; detector, PDA. The configurations of the structures were tentatively assigned.

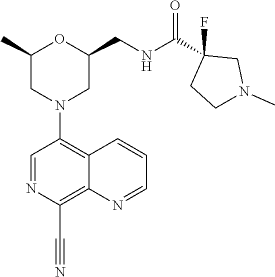

Isomer 1: MS: 413 [M+H]⁺. ¹H NMR (400 MHz, DMSO-d₆) δ 9.19 (d, J=4.1 Hz, 1H), 8.54 (d, J=8.7 Hz, 1H), 8.36 (s, 1H), 8.27 (s, 1H), 7.87 (dd, J=9.0, 4.1 Hz, 1H), 3.96 (q, J=7.9 Hz, 2H), 3.49 (t, J=11.1 Hz, 2H), 3.29-3.16 (m, 2H), 2.83 (dq, J=33.8, 11.2 Hz, 4H), 2.70-2.55 (m, 2H), 2.32 (d, J=9.7 Hz, 2H), 2.24 (s, 3H), 2.09-1.91 (m, 1H), 1.18 (d, J=6.1 Hz, 3H).

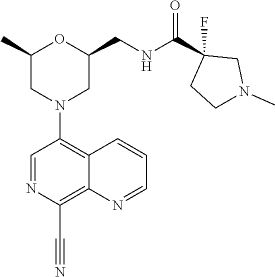

Isomer 2: MS: 413 [M+H]⁺. ¹H NMR (400 MHz, DMSO-d₆) δ 9.19 (d, J=4.0 Hz, 1H), 8.54 (d, J=8.7 Hz, 1H), 8.36 (s, 1H), 8.27 (s, 1H), 7.86 (dd, J=8.8, 4.0 Hz, 1H), 3.95 (d, J=10.0 Hz, 2H), 3.50 (t, J=10.1 Hz, 2H), 3.29-3.15 (m, 2H), 2.77 (dddd, J=48.2, 40.4, 18.9, 11.1 Hz, 6H), 2.29 (d, J=27.6 Hz, 5H), 2.12-1.89 (m, 1H), 1.18 (d, J=6.2 Hz, 3H).

### Example 336 (Isomer 1): (3R)—N-{[(2S,6R)-4-(8-cyano-1,7-naphthyridin-5-yl)-6-methylmorpholin-2-yl]methyl}-3-fluoropyrrolidine-3-carboxamide & Example 337 (Isomer 2): (3S)—N-{[(2S,6R)-4-(8-cyano-1,7-naphthyridin-5-yl)-6-methylmorpholin-2-yl]methyl}-3-fluoropyrrolidine-3-carboxamide

The two isomers were obtained by the SFC chiral separation of N-{[(2S,6R)-4-(8-cyano-1,7-naphthyridin-5-yl)-6-methylmorpholin-2-yl]methyl}-3-fluoropyrrolidine-3-carboxamide The SFC conditions are: column, ADH, Prep SFC-P100; mobile phase, methanol+20 mM NH₄OH, 45° C./80 bar, 100 g/min; detector, PDA. The configurations of the structures were tentatively assigned.

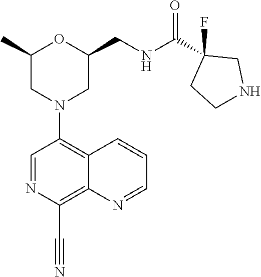 [SMILES: C[C@@H]1CN(c2cnc(C#N)c3ncccc23)C[C@H](CNC(=O)[C@@]2(F)CCNC2)O1]

Isomer 1: MS: 399 [M+H]. ¹H NMR (400 MHz, CDCl₃) δ 9.18 (dd, J=4.0, 1.9 Hz, 1H), 8.39 (d, J=8.5 Hz, 1H), 8.30 (d, J=1.3 Hz, 1H), 7.75-7.66 (m, 1H), 6.95 (d, J=6.3 Hz, 1H), 4.07-3.97 (m, 2H), 3.75-3.64 (m, 1H), 3.46-3.12 (m, 6H), 2.81 (td, J=10.9, 2.6 Hz, 2H), 2.48-2.40 (m, 1H), 2.23-2.08 (m, 1H), 1.31-1.26 (m, 4H).

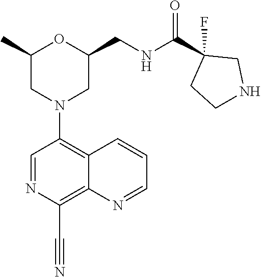 [SMILES: C[C@@H]1CN(c2cnc(C#N)c3ncccc23)C[C@H](CNC(=O)[C@]2(F)CCNC2)O1]

Isomer 2: MS: 399 [M+H]⁺. ¹H NMR (400 MHz, CDCl₃) δ 9.18 (dd, J=3.9, 1.9 Hz, 1H), 8.39 (d, J=8.7 Hz, 1H), 8.30 (s, 1H), 7.70 (dd, J=8.7, 4.1 Hz, 1H), 6.94 (d, J=6.5 Hz, 1H), 4.07-3.99 (m, 2H), 3.73-3.67 (m, 1H), 3.41-3.16 (m, 6H), 2.82 (td, J=11.1, 5.4 Hz, 2H), 2.50-2.35 (m, 1H), 2.27-2.08 (m, 1H), 1.31-1.26 (m, 4H).

### Example 338 (Isomer 1): (3R)—N-{[(2S,6R)-4-(8-cyano-1,7-naphthyridin-5-yl)-6-methylmorpholin-2-yl]methyl}-3-fluoropiperidine-3-carboxamide & Example 339 (Isomer 2): (3S)—N-{[(2S,6R)-4-(8-cyano-1,7-naphthyridin-5-yl)-6-methylmorpholin-2-yl]methyl}-3-fluoropiperidine-3-carboxamide

The two isomers were obtained by SFC chiral separation of N-{[(2S,6R)-4-(8-cyano-1,7-naphthyridin-5-yl)-6-methylmorpholin-2-yl]methyl}-3-fluoropiperidine-3-carboxamide. The SFC conditions are: column, ADH, Prep SFC-P100; mobile phase, methanol+20 mM NH₄OH, 45° C./80 bar, 100 g/min; detector, PDA. The configurations of the structures were tentatively assigned.

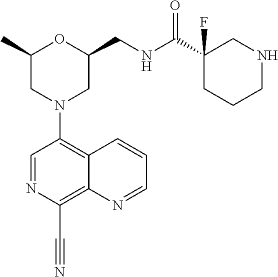 [SMILES: C[C@@H]1CN(c2cnc(C#N)c3ncccc23)C[C@H](CNC(=O)[C@@]2(F)CCCNC2)O1]

Isomer 1: MS: 413 [M+H]⁺. H NMR (400 MHz, CDCl₃) δ 9.17 (dd, J=4.0, 1.7 Hz, 1H), 8.38 (dd, J=8.7, 1.6 Hz, 1H), 8.30 (s, 1H), 7.69 (dd, J=8.6, 4.1 Hz, 1H), 6.89 (d, J=6.2 Hz, 1H), 4.05-3.98 (m, 2H), 3.71-3.59 (m, 1H), 3.39-3.24 (m, 2H), 3.16 (dd, J=33.4, 14.4 Hz, 1H), 3.06-2.99 (m, 1H), 2.84-2.67 (m, 3H), 2.70 (t, J=12.5 Hz, 1H), 2.28-2.08 (m, 1H), 2.0-1.93 (m, 1H), 1.76-1.61 (m, 2H), 1.38-1.18 (m, 4H).

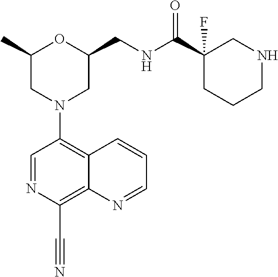 [SMILES: C[C@@H]1CN(c2cnc(C#N)c3ncccc23)C[C@H](CNC(=O)[C@]2(F)CCCNC2)O1]

Isomer 2:MS: 413 [M+H]⁺. ¹H NMR (400 MHz, CDCl₃) δ 9.20 (dd, J=4.0, 1.7 Hz, 1H), 8.41 (dd, J=8.7, 1.5 Hz, 1H), 8.32 (s, 1H), 7.72 (dd, J=8.6, 4.1 Hz, 1H), 6.92 (d, J=6.3 Hz, 1H), 4.08-4.0 (m, 2H), 3.69-3.63 (m, 1H), 3.41-3.27 (m, 3H), 3.26-3.03 (m, 3H), 2.87-2.67 (m, 3H), 2.29-2.09 (m, 1H), 2.0-1.93 (m, 1H), 1.76-1.61 (m, 1H), 1.37-1.26 (m, 4H).

### Example 340 (Isomer 1): (2R)—N-{[(2S,6R)-4-(8-cyanoquinoxalin-5-yl)-6-methylmorpholin-2-yl]methyl}-2-hydroxypropanamide & Example 341 (Isomer 2): (2S)—N-{[(2S,6R)-4-(8-cyanoquinoxalin-5-yl)-6-methylmorpholin-2-yl]methyl}-2-hydroxypropanamide

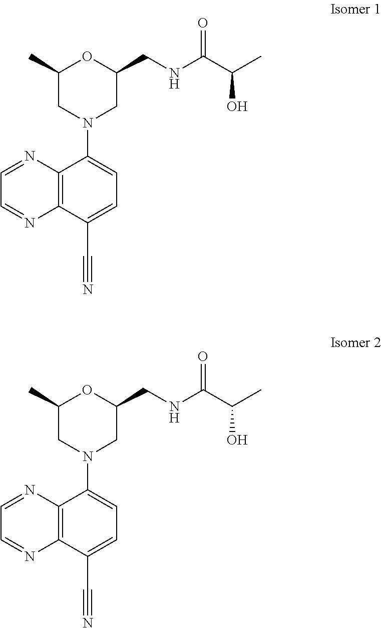 [SMILES: C[C@@H]1CN(c2ccc(C#N)c3nccnc23)C[C@H](CNC(=O)[C@@H](C)O)O1 ; C[C@@H]1CN(c2ccc(C#N)c3nccnc23)C[C@H](CNC(=O)[C@H](C)O)O1]

The title compounds were made from 8-[(2R,6R)-2-(hydroxymethyl)-6-methylmorpholin-4-yl]quinoxaline-5-carbonitrile and lactic acid. The two isomers were obtained from SFC chiral separation. The SFC conditions are column, column, AS-H, Prep SFC-P100; mobile phase, methanol+20 mM NH₄OH, 40° C./80 bar, 100 g/min; detector, PDA. The configurations of the structures were tentatively assigned.

Isomer 1: MS:356 [M+H]⁺. ¹H NMR (400 MHz, DMSO-d₆) δ 9.06 (d, J=1.8 Hz, 1H), 8.95 (d, J=1.8 Hz, 1H), 8.25 (d, J=8.4 Hz, 1H), 7.76 (t, J=6.1 Hz, 1H), 7.20 (d, J=8.5 Hz, 1H), 5.52 (br s, 1H), 4.20 (d, J=12.3 Hz, 1H), 4.15-4.10 (d, J=12.3 Hz, 1H), 3.98 (q, J=6.8 Hz, 1H), 3.91-3.77 (m, 1H), 3.24 (t, J=6.2 Hz, 2H), 2.73 (ddd, J=12.5, 10.4, 2.5 Hz, 2H), 1.21-1.17 (m, 6H).

Isomer 2: MS:356 [M+H]⁺. ¹H NMR (400 MHz, DMSO-d₆) δ 9.06 (d, J=1.8 Hz, 1H), 8.95 (d, J=1.8 Hz, 1H), 8.25 (d, J=8.4 Hz, 1H), 7.76 (t, J=6.0 Hz, 1H), 7.20 (d, J=8.4 Hz, 1H), 5.53 (br s, 1H), 4.20 (d, J=12.2 Hz, 1H), 4.13 (d, J=12.2 Hz, 1H), 4.03-3.95 (m, 1H), 3.85 (dt, J=12.9, 6.2 Hz, 1H), 3.24 (t, J=6.1 Hz, 2H), 2.72 (dd, J=12.3, 10.4 Hz, 2H), 1.23-1.17 (m, 6H).

### Example 342: N-{[(2S,6R)-6-methyl-4-(8-methylquinolin-5-yl)morpholin-2-yl]methyl}-2-(1-methylpiperidin-4-yl)acetamide

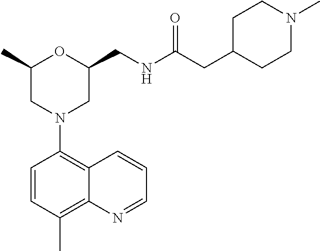

The title compounds were made from [(2R,6R)-6-methyl-4-(8-methylquinolin-5-yl)morpholin-2-yl]methanol and 2-(1-methylpiperidin-4-yl)acetic acid. MS:411 [M+H]⁺. ¹HNMR (400 MHz, CDCl₃) δ 8.94 (dd, J=4.1, 1.7 Hz, 1H), 8.50 (dd, J=8.5, 1.8 Hz, 1H), 7.47 (d, J=7.6 Hz, 1H), 7.40 (dd, J=8.5, 4.2 Hz, 1H), 7.02 (d, J=7.5 Hz, 1H), 5.88 (t, J=5.8 Hz, 1H), 4.04-3.95 (m, 2H), 3.65 (ddd, J=13.9, 7.0, 3.6 Hz, 1H), 3.22-3.16 (m, 1H), 3.12 (t, J=11.5 Hz, 2H), 2.84 (d, J=11.2 Hz, 2H), 2.75 (s, 3H), 2.58 (td, J=10.9, 4.4 Hz, 2H), 2.27 (s, 3H), 2.13 (d, J=7.0 Hz, 2H), 1.96 (td, J=11.8, 2.4 Hz, 2H), 1.86-1.78 (m, 1H), 1.74 (d, J=13.1 Hz, 2H), 1.33 (q, J=12.1 Hz, 2H), 1.25 (d, J=6.2 Hz, 3H).

### Example 343: N-{[(2S,6R)-4-(8-cyanoquinazolin-5-yl)-6-methylmorpholin-2-yl]methyl}-3-fluoropyrrolidine-3-carboxamide

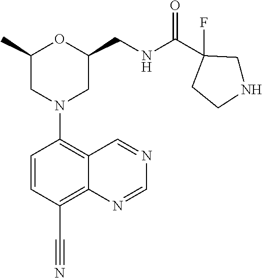 [SMILES: C[C@@H]1CN(c2ccc(C#N)c3ncncc23)C[C@H](CNC(=O)C2(F)CCNC2)O1]

The title compounds were made from [(2R,6R)-4-(8-cyanoquinazolin-5-yl)-6-methylmorpholin-2-yl]methyl 4-methylbenzene-1-sulfonate and 1-[(tert-butoxy)carbonyl]-3-fluoropyrrolidine-3-carboxylic acid. MS:399 [M+H]⁺. ¹H NMR (400 MHz, CDCl₃) δ 9.60 (s, 1H), 9.44 (s, 1H), 8.17 (dd, J=8.2, 0.8 Hz, 1H), 7.08 (d, J=8.1 Hz, 1H), 6.92 (d, J=6.3 Hz, 1H), 4.11-4.01 (m, 2H), 3.71-3.65 (m, 1H), 3.45-3.32 (m, 3H), 3.30-3.08 (m, 4H), 2.84 (ddd, J=12.1, 10.4, 1.6 Hz, 1H), 2.76 (dd, J=12.3, 10.2 Hz, 1H), 2.46-2.31 (m, 1H), 2.23-2.05 (m, 1H), 1.29 (d, J=6.2 Hz, 3H).

### Example 344 (Isomer 1): (3R)—N-{[(2S,6R)-4-(8-cyanoquinazolin-5-yl)-6-methylmorpholin-2-yl]methyl}-3-fluoropyrrolidine-3-carboxamide & Example 345 (Isomer 2): (3S)—N-{[(2S,6R)-4-(8-cyanoquinazolin-5-yl)-6-methylmorpholin-2-yl]methyl}-3-fluoropyrrolidine-3-carboxamide

The two isomers were obtained by SFC chiral separation of N-{[(2S,6R)-4-(8-cyanoquinazolin-5-yl)-6-methylmorpholin-2-yl]methyl}-3-fluoropyrrolidine-3-carboxamide. The SFC conditions are: column, IG-H, Prep SFC-P100; mobile phase, methanol+20 mM NH₄OH, 45° C./80 bar, 100 g/min; detector, PDA. The configurations of the structures were tentatively assigned.

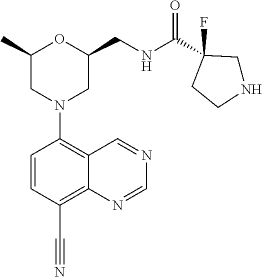 [SMILES: C[C@@H]1CN(c2ccc(C#N)c3ncncc23)C[C@H](CNC(=O)[C@@]2(F)CCNC2)O1]

Isomer 1: MS:399 [M+H]⁺. ¹H NMR (400 MHz, CDCl₃) δ 9.60 (s, 1H), 9.45 (d, J=1.2 Hz, 1H), 8.17 (d, J=8.1 Hz, 1H), 7.08 (d, J=8.1 Hz, 1H), 6.92 (d, J=6.5 Hz, 1H), 4.11-4.0 (m, 2H), 3.74-3.62 (m, 1H), 3.46-3.39 (m, 2H), 3.38-3.33 (m, 1H), 3.29-3.24 (m, 1H), 3.32-3.09 (m, 2H), 2.84 (t, J=11.2 Hz, 1H), 2.76 (t, J=11.2 Hz, 1H), 2.46-2.31 (m, 1H), 2.21-2.05 (m, 1H), 1.33-1.24 (m, 4H).

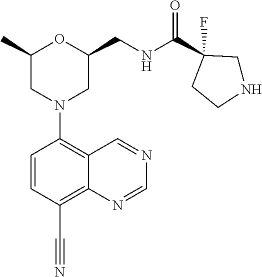 [SMILES: C[C@@H]1CN(c2ccc(C#N)c3ncncc23)C[C@H](CNC(=O)[C@]2(F)CCNC2)O1]

Isomer 2: MS:399 [M+H]⁺. ¹H NMR (400 MHz, CDCl₃) δ 9.60 (d, J=1.3 Hz, 1H), 9.45 (d, J=1.3 Hz, 1H), 8.18 (dd, J=8.2, 1.3 Hz, 1H), 7.08 (dd, J=8.2, 1.2 Hz, 1H), 6.92 (d, J=6.3 Hz, 1H), 4.14-3.96 (m, 2H), 3.74-3.63 (m, 1H), 3.47-3.39 (m, 2H), 3.39-3.31 (m, 1H), 3.28-3.10 (m, 3H), 2.84 (t, J=11.3 Hz, 1H), 2.76 (t, J=11.2 Hz, 1H), 2.39 (ddt, J=30.5, 15.2, 7.9 Hz, 1H), 2.16 (ddt, J=26.9, 13.4, 6.2 Hz, 1H), 1.34-1.24 (m, 4H).

### Example 346: (2R,6R)-4-(8-Cyano-quinazolin-5-yl)-6-methyl-morpholine-2-carboxylic acid (4-fluoro-1-methyl-piperidin-4-ylmethyl)-amide

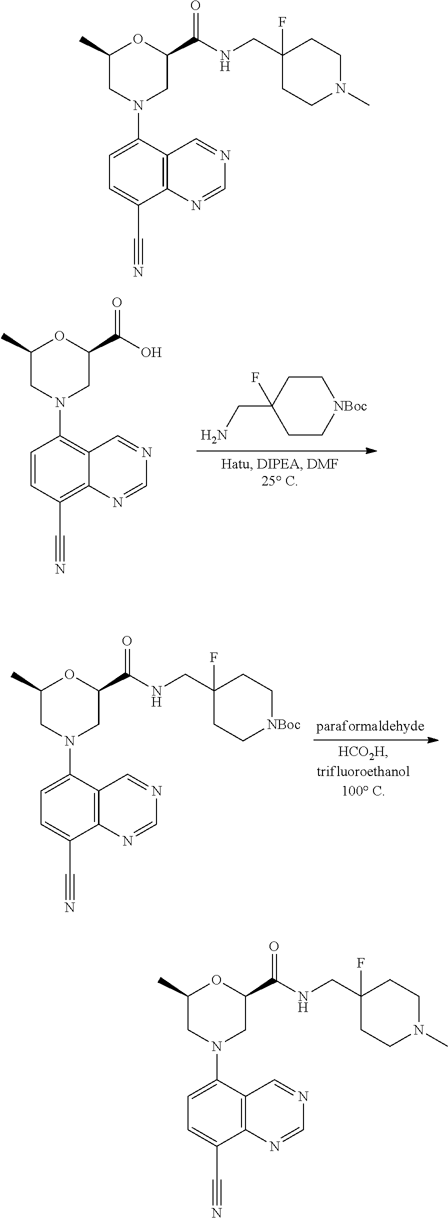

4-({[(2R,6R)-4-(8-Cyano-quinazolin-5-yl)-6-methyl-morpholine-2-carbonyl]-amino}-methyl)-4-fluoro-piperidine-1-carboxylic acid tert-butyl ester: Into a 50 mL round-bottom flask, was placed (2R,6R)-4-(8-Cyano-quinazolin-5-yl)-6-methyl-morpholine-2-carboxylic acid (40.0 mg; 0.121 mmol; 1.0 eq.) in DMF (2.0 ml). Hatu (68.8 mg; 0.181 mmol; 1.50 eq.) was added and the resulting solution stirred for 10 minutes at rt after which tert-butyl 4-(aminomethyl)-4-fluoropiperidine-1-carboxylate (42.1 mg; 0.181 mmol; 1.50 eq.) and DIPEA (63.1 μl; 0.362 mmol; 3.0 eq.) were respectively added. The resulting mixture was stirred at room temperature for 2 hours. LCMS showed the reaction was complete. It was filtered through celite and concentrated under vacuum. The residue was purified by chromatography on a Biotage (PuriFlash Column, 15μ Si HP, 10 g) with ethyl acetate/petroleum ether (10:900 to 70:30) for 18 minutes to yield the title compound (46.0 mg; 74.4%) as a yellow oil. MS: 513 [M+H]⁺.

(2R,6R)-4-(8-Cyano-quinazolin-5-yl)-6-methyl-morpholine-2-carboxylic acid (4-fluoro-1-methyl-piperidin-4-ylmethyl)-amide: To a solution of 4-({[(2R,6R)-4-(8-Cyano-quinazolin-5-yl)-6-methyl-morpholine-2-carbonyl]-amino}-methyl)-4-fluoro-piperidine-1-carboxylic acid tert-butyl ester (46.0 mg; 0.090 mmol; 1.0 eq.) in 2,2,2-trifluoroethanol (2.0 ml) was added paraformaldehyde (32.3 mg; 0.359 mmol; 4.0 eq.), and formic acid (67.7 μl; 1.795 mmol; 20.0 eq.). The mixture was stirred at 100° C. under microwave for 30 minutes. LCMS showed the reaction was complete with major desired product. The volatile was evaporated and 3 mL of DMSO added. The product was purified on reverse phase system using a gradient of 05-45% CH₃CN/H₂O (0.1% Ammonium Hydroxide) in 2 injections of 2 mL each. The desired fractions were evaporated to provide the title compound (13.1 mg; 34.2%) as a yellow solid. MS: 427 [M+H]⁺. ¹H NMR (400 MHz, Methanol-d₄) δ 9.75 (s, 1H), 9.35 (s, 1H), 8.34 (dd, J=8.2, 2.3 Hz, 1H), 7.35 (dd, J=8.2, 2.2 Hz, 1H), 4.57 (dd, J=10.6, 3.0 Hz, 1H), 4.19 (d, J=8.7 Hz, 1H), 3.87 (d, J=12.3 Hz, 1H), 3.59 (d, J=12.5 Hz, 1H), 3.54-3.38 (m, 2H), 3.0 (t, J=11.5 Hz, 1H), 2.88 (t, J=11.4 Hz, 1H), 2.73 (d, J=11.8 Hz, 2H), 2.46-2.25 (m, 5H), 1.79 (dd, J=44.8, 12.3 Hz, 4H), 1.37 (d, J=5.8 Hz, 3H).

The following compounds were synthesized in an analogous manner.

### Example 347: (2R,6R)-4-(8-Cyano-quinazolin-5-yl)-6-methyl-morpholine-2-carboxylic acid (5-methyl-5-aza-spiro[3.5]non-8-yl)-amide

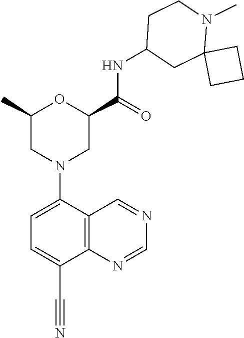 [SMILES: C[C@@H]1CN(c2ccc(C#N)c3ncncc23)C[C@H](C(=O)NC2CCN(C)C3(CCC3)C2)O1]

The title compound was prepared from (2R,6R)-4-(8-Cyano-quinazolin-5-yl)-6-methyl-morpholine-2-carboxylic acid and tert-butyl 8-amino-5-azaspiro[3.5]nonane-5-carboxylate. MS: 435 [M+H]⁺. ¹H NMR (400 MHz, Methanol-d₄) δ 9.74 (s, 1H), 9.35 (s, 1H), 8.34 (d, J=8.2 Hz, 1H), 7.34 (d, J=8.3 Hz, 1H), 4.50 (d, J=10.6 Hz, 1H), 4.18 (t, J=8.2 Hz, 1H), 4.03-3.82 (m, 2H), 3.59 (d, J=12.4 Hz, 1H), 2.99 (td, J=11.6, 4.2 Hz, 1H), 2.87 (t, J=11.4 Hz, 1H), 2.73 (d, J=13.5 Hz, 1H), 2.56 (p, J=7.7, 6.0 Hz, 1H), 2.44-2.30 (m, 4H), 2.23 (q, J=9.7 Hz, 1H), 2.16-2.05 (m, 1H), 1.97-1.57 (m, 7H), 1.37 (d, J=6.0 Hz, 3H).

### Example 348: (2R,6R)-4-(8-Cyano-[1,7]naphthyridin-5-yl)-6-methyl-morpholine-2-carboxylic acid (4-fluoro-1-methyl-piperidin-4-ylmethyl)-amide

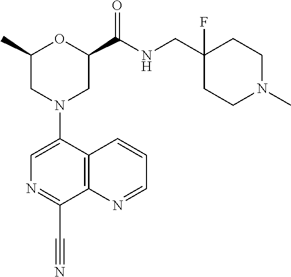

The title compound was prepared from (2R,6R)-4-(8-Cyano-[1,7]naphthyridin-5-yl)-6-methyl-morpholine-2-carboxylic acid and tert-butyl 4-(aminomethyl)-4-fluoropiperidine-1-carboxylate. MS: 427 [M+H]⁺. ¹H NMR (400 MHz, Methanol-d₄) δ 9.15 (dd, J=4.1, 1.6 Hz, 1H), 8.68 (d, J=8.6 Hz, 1H), 8.40 (s, 1H), 7.86 (dd, J=8.8, 4.2 Hz, 1H), 4.56 (dd, J=10.9, 2.6 Hz, 1H), 4.25-4.09 (m, 1H), 3.84 (d, J=12.1 Hz, 1H), 3.59-3.45 (m, 3H), 2.94 (dt, J=26.0, 11.4 Hz, 2H), 2.71 (d, J=11.6 Hz, 2H), 2.31 (s, 5H), 1.91-1.66 (m, 4H), 1.38 (d, J=6.2 Hz, 3H).

### Example 349 (Isomer 1): (2S,6R)-4-(8-cyano-1,7-naphthyridin-5-yl)-N-[[(3R)-3-fluoro-1-methylpyrrolidin-3-yl]methyl]-6-(trifluoromethyl)morpholine-2-carboxamide & Example 350 (Isomer 2): (2R,6S)-4-(8-cyano-1,7-naphthyridin-5-yl)-N-[[(3R)-3-fluoro-1-methylpyrrolidin-3-yl]methyl]-6-(trifluoromethyl)morpholine-2-carboxamide

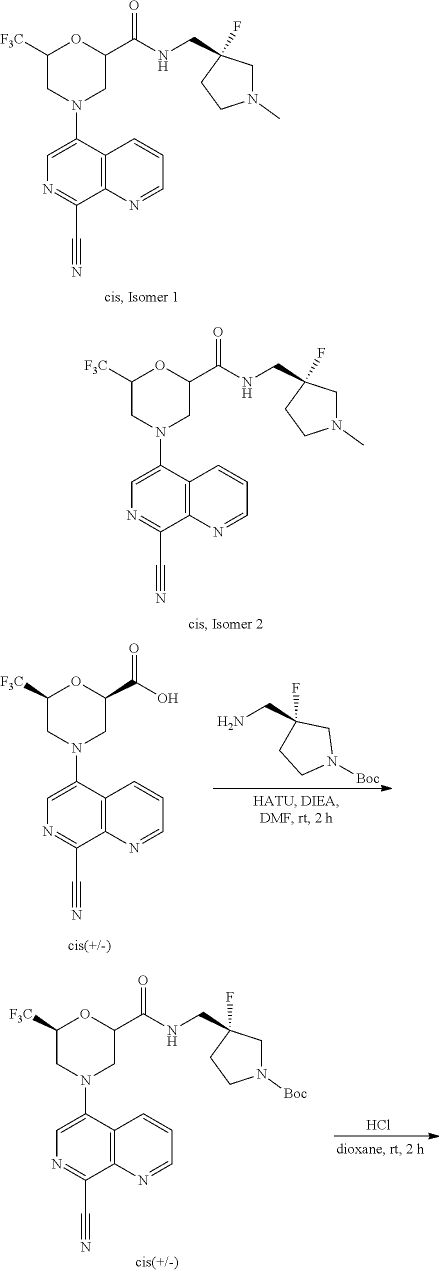 [SMILES: CN1CC[C@@](F)(CNC(=O)C2CN(c3cnc(C#N)c4ncccc34)CC(C(F)(F)F)O2)C1 ; CN1CC[C@@](F)(CNC(=O)C2CN(c3cnc(C#N)c4ncccc34)CC(C(F)(F)F)O2)C1 ; N#Cc1ncc(N2C[C@@H](C(F)(F)F)O[C@@H](C(=O)O)C2)c2cccnc12 ; CC(C)(C)OC(=O)N1CC[C@@](F)(CN)C1 ; CC(C)(C)OC(=O)N1CC[C@@](F)(CNC(=O)C2CN(c3cnc(C#N)c4ncccc34)C[C@@H](C(F)(F)F)O2)C1 ; Cl]

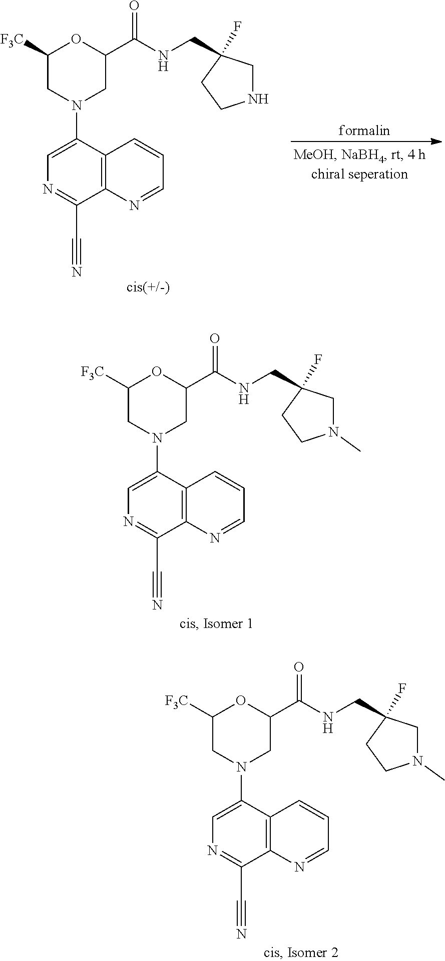 [SMILES: N#Cc1ncc(N2CC(C(=O)NC[C@]3(F)CCNC3)O[C@H](C(F)(F)F)C2)c2cccnc12 ; "formalin" ; CN1CC[C@@](F)(CNC(=O)C2CN(c3cnc(C#N)c4ncccc34)CC(C(F)(F)F)O2)C1 ; CN1CC[C@@](F)(CNC(=O)C2CN(c3cnc(C#N)c4ncccc34)CC(C(F)(F)F)O2)C1]

tert-butyl (3R)-3-([[cis-4-(8-cyano-1,7-naphthyridin-5-yl)-6-(trifluoromethyl)morpholin-2-yl]formamido]methyl)-3-fluoropyrrolidine-1-carboxylate: To a solution of cis-4-(8-cyano-1,7-naphthyridin-5-yl)-6-(trifluoromethyl)morpholine-2-carboxylic acid (72 mg, 0.20 mmol) in DMF (4 mL) was added tert-butyl (3R)-3-(aminomethyl)-3-fluoropyrrolidine-1-carboxylate (88 mg, 0.41 mmol), HATU (153 mg, 0.41 mmol) and DIEA (131 mg, 1.01 mmol) in sequence at room temperature. The resulting mixture was stirred for 2 h at room temperature. When the reaction was done, the reaction was then diluted with water (30 mL). The resulting mixture was extracted with ethyl acetate (100 mL×3). The organic phases were combined, washed with brine and dried over Na₂SO₄. The solvent was removed under reduced pressure to yield the title compound as a yellow solid (100 mg, crude), which was used directly in next step without further purification. MS: 553 [M+H]⁺.

cis-4-(8-cyano-1,7-naphthyridin-5-yl)-N-[[(3S)-3-fluoropyrrolidin-3-yl]methyl]-6-(trifluoromethyl)morpholine-2-carboxamide: To a solution of tert-butyl (3R)-3-([[cis-4-(8-cyano-1,7-naphthyridin-5-yl)-6-(trifluoromethyl)morpholin-2-yl]formamido]methyl)-3-fluoropyrrolidine-1-carboxylate (100 mg, crude) in dioxane (5 mL) was added HCl solution (6 N in water, 1 mL, 6.0 mmol) at room temperature. The resulting mixture was stirred for 2 h at room temperature. After the reaction was done, the reaction mixture was concentrated under reduced pressure to yield the title compound as a yellow solid (100 mg, crude), which was used directly in next step without further purification. MS: 453 [M+H]⁺.

(2S,6R)-4-(8-cyano-1,7-naphthyridin-5-yl)-N-[[(3R)-3-fluoro-1-methylpyrrolidin-3-yl]methyl]-6-(trifluoromethyl)morpholine-2-carboxamide & (2R,6S)-4-(8-cyano-1,7-naphthyridin-5-yl)-N-[[(3R)-3-fluoro-1-methylpyrrolidin-3-yl]methyl]-6-(trifluoromethyl)morpholine-2-carboxamide: To a solution of cis-4-(8-cyano-1,7-naphthyridin-5-yl)-N-[[(3S)-3-fluoropyrrolidin-3-yl]methyl]-6-(trifluoromethyl)morpholine-2-carboxamide (100 mg, crude) in MeOH (6 mL) was added formalin solution (37%, 4.2 mL) and NaBH₄ (60 mg, 1.59 mmol) in sequence at room temperature. The resulting mixture was stirred for 4 h at room temperature. After the reaction was done, the reaction mixture was concentrated under reduced pressure and the residue was first purified by prep-HPLC under the following conditions: column, XBridge Prep C18 OBD Column, 150×19 mm, 5 um; mobile phase, acetonitrile in water (with 10 mmol/L NH₄HCO₃ and 0.1% NH₃.H₂O), 28% to 52% gradient in 8 min; detector, UV 254 nm. The two isomers were obtained by separation on chiral-HPLC under the following conditions: column, CHIRAL Cellulose-SB, 0.46×10 cm, 3 um; mobile phase, hexane (with 0.1% DEA) in EtOH, 50% isocratic in 25 min; detector, UV 254 nm.

Isomer 1: MS: 467 [M+H]⁺. ¹H NMR (400 MHz, DMSO-d₆, ppm) δ 9.25-9.19 (m, 1H), 8.76-8.69 (m, 1H), 8.56 (s, 1H), 8.16-8.12 (m, 1H), 7.99-7.91 (m, 1H), 4.86-4.81 (m, 1H), 4.71-4.64 (m, 1H), 3.70-3.63 (m, 2H), 3.58-3.40 (m, 2H), 3.30-3.22 (m, 2H), 3.19-3.08 (m, 1H), 2.69-2.52 (m, 2H), 2.41-2.31 (m, 1H), 2.21 (s, 3H), 2.10-1.79 (m, 2H).

Isomer 2: MS: 467 [M+H]⁺. ¹H NMR (400 MHz, DMSO-d₆, ppm) δ 9.17-9.06 (m, 1H), 8.73-8.66 (m, 1H), 8.57 (s, 1H), 8.13-8.07 (m, 1H), 8.01-7.92 (m, 1H), 4.85-4.78 (m, 1H), 4.69-4.63 (m, 1H), 3.73-3.65 (m, 2H), 3.55-3.39 (m, 2H), 3.31-3.23 (m, 2H), 3.20-3.08 (m, 1H), 2.67-2.51 (m, 2H), 2.43-2.34 (m, 1H), 2.21 (s, 3H), 2.08-1.76 (m, 2H).

The following compounds were synthesized in an analogous manner.

### Example 351 (Isomer 1): (2S,6R)-4-(8-cyano-1,7-naphthyridin-5-yl)-N-((4-fluoro-1-methylpiperidin-4-yl)methyl)-6-(trifluoromethyl)morpholine-2-carboxamide & Example 352 (Isomer 2): (2R,6S)-4-(8-cyano-1,7-naphthyridin-5-yl)-N-((4-fluoro-1-methylpiperidin-4-yl)methyl)-6-(trifluoromethyl)morpholine-2-carboxamide

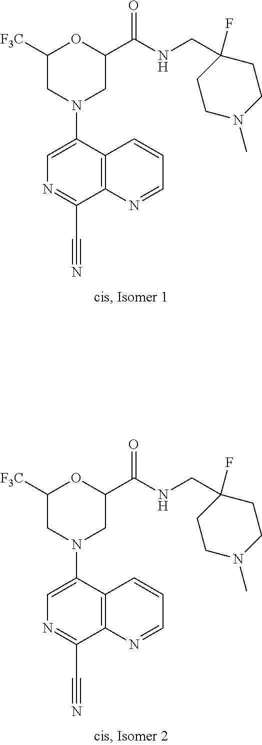 [SMILES: CN1CCC(F)(CNC(=O)C2CN(c3cnc(C#N)c4ncccc34)CC(C(F)(F)F)O2)CC1 ; CN1CCC(F)(CNC(=O)C2CN(c3cnc(C#N)c4ncccc34)CC(C(F)(F)F)O2)CC1]

The title compounds were made from tert-butyl 4-([[cis-4-(8-cyano-1,7-naphthyridin-5-yl)-6-(trifluoromethyl)morpholin-2-yl]formamido]methyl)-4-fluoropiperidine-1-carboxylate. The two isomers were obtained by separation on chiral-HPLC under the following conditions: column, CHIRALPAK Cellulose-SB, 0.46×10 cm, 3 um; mobile phase, hexane (with 0.1% DEA) in EtOH, 50% isocratic in 25 min; detector, UV 254 nm.

Isomer 1: MS: 481 [M+H]⁺. ¹H NMR (400 MHz, DMSO-d₆, ppm) δ 9.25-9.19 (m, 1H), 8.76-8.69 (m, 1H), 8.56 (s, 1H), 8.08-8.01 (m, 1H), 7.99-7.91 (m, 1H), 4.87-4.78 (m, 1H), 4.72-4.65 (m, 1H), 3.71-3.62 (m, 2H), 3.53-3.22 (m, 3H), 3.22-3.03 (m, 1H), 2.57-2.53 (m, 2H), 2.16 (s, 3H), 2.14-2.05 (m, 2H), 1.79-1.49 (m, 4H).

Isomer 2: MS: 481 [M+H]⁺. ¹H NMR (400 MHz, DMSO-d₆, ppm) δ 9.20-9.15 (m, 1H), 8.72-8.64 (m, 1H), 8.55 (s, 1H), 8.05-7.90 (m, 2H), 4.90-4.79 (m, 1H), 4.71-4.66 (m, 1H), 3.70-3.61 (m, 2H), 3.53-3.03 (m, 4H), 2.60-2.55 (m, 2H), 2.18 (s, 3H), 2.15-2.03 (m, 2H), 1.80-1.45 (m, 4H).

### Example 353 (Isomer 1): (2R,6S)-4-(8-cyanoquinolin-5-yl)-N-[(4-fluoro-1-methylpiperidin-4-yl)methyl]-6-(trifluoromethyl)morpholine-2-carboxamide & Example 354 (Isomer 2): (2S,6R)-4-(8-cyanoquinolin-5-yl)-N-[(4-fluoro-1-methylpiperidin-4-yl)methyl]-6-(trifluoromethyl)morpholine-2-carboxamide

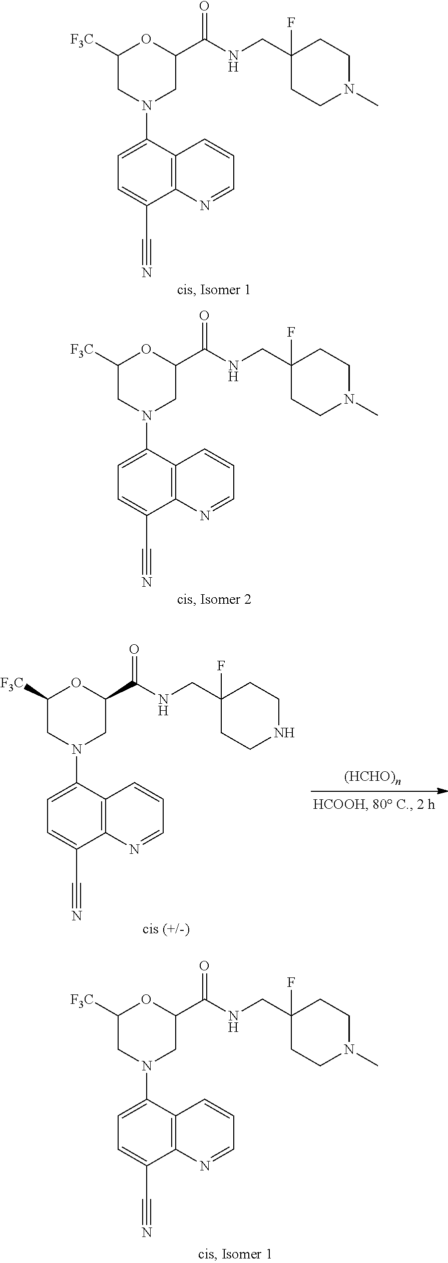 [SMILES: CN1CCC(F)(CNC(=O)C2CN(c3ccc(C#N)c4ncccc34)CC(C(F)(F)F)O2)CC1 ; CN1CCC(F)(CNC(=O)C2CN(c3ccc(C#N)c4ncccc34)CC(C(F)(F)F)O2)CC1 ; N#Cc1ccc(N2C[C@@H](C(F)(F)F)O[C@@H](C(=O)NCC3(F)CCNCC3)C2)c2cccnc12 ; CN1CCC(F)(CNC(=O)C2CN(c3ccc(C#N)c4ncccc34)CC(C(F)(F)F)O2)CC1]

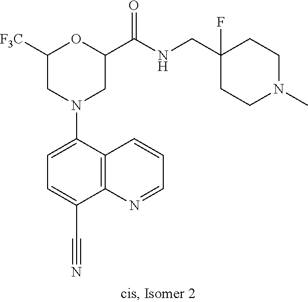 [SMILES: CN1CCC(F)(CNC(=O)C2CN(c3ccc(C#N)c4ncccc34)CC(C(F)(F)F)O2)CC1]

To a solution cis-4-(8-cyanoquinolin-5-yl)-N-[(4-fluoropiperidin-4-yl)methyl]-6-(trifluoromethyl)morpholine-2-carboxamide (60 mg, 0.13 mmol) in HCOOH (5 mL) was added (HCHO)ₙ (285 mg, 3.16 mmol) at room temperature. The resulting mixture was stirred for 3 h at 100° C. After the reaction was done, the reaction mixture was concentrated under reduced pressure and the residue was first purified by prep-HPLC under the following conditions: column, XBridge Prep C18 OBD Column, 150×19 mm, 5 um; mobile phase, acetonitrile in water (with 10 mmol/L NH₄HCO₃ and 0.1% NH₃.H₂O), 32% to 62% gradient in 8 min; detector, UV 254 nm. Then the two enantiomeric products were obtained by separation on chiral-HPLC under the following conditions: column, CHIRALPAK IG-3, 0.46×5 cm, 3 um; mobile phase, hexane/DCM (5:1, with 0.1% DEA) in EtOH, 50% isocratic in 25 min; detector, UV 254 nm.

Isomer 1: MS: 480 [M+H]⁺. ¹H NMR (300 MHz, Methanol-d₄, ppm) δ 9.41-9.32 (m, 1H), 9.28-9.20 (m, 1H), 8.47 (d, J=8.1 Hz, 1H), 8.19-8.08 (m, 1H), 7.65 (d, J=8.2 Hz, 1H), 4.87-4.77 (m, 2H), 3.85-3.71 (m, 1H), 3.71-3.43 (m, 5H), 3.35-3.09 (m, 4H), 2.89 (s, 3H), 2.16-1.99 (m, 4H).

Isomer 2: MS: 480 [M+H]⁺. ¹H NMR (300 MHz, Methanol-d₄, ppm) δ 9.37 (d, J=8.4 Hz, 1H), 9.24 (m, 1H), 8.47 (d, J=8.1 Hz, 1H), 8.14 (m, 1H), 7.65 (d, J=8.2 Hz, 1H), 4.82 (d, J=10.2 Hz, 2H), 3.80 (d, J=12.4 Hz, 1H), 3.75-3.40 (m, 5H), 3.32 (s, 1H), 3.26-2.98 (m, 3H), 2.89 (s, 3H), 2.19-1.91 (m, 4H).

The following compounds were synthesized in an analogous manner.

### Example 355 (Isomer 1): (2R,6S)-4-(8-cyanoquinoxalin-5-yl)-N—(((R)-3-fluoro-1-methylpyrrolidin-3-yl)methyl)-6-(trifluoromethyl)morpholine-2-carboxamide & Example 356 (Isomer 2): (2S,6R)-4-(8-cyanoquinoxalin-5-yl)-N—(((R)-3-fluoro-1-methylpyrrolidin-3-yl)methyl)-6-(trifluoromethyl)morpholine-2-carboxamide

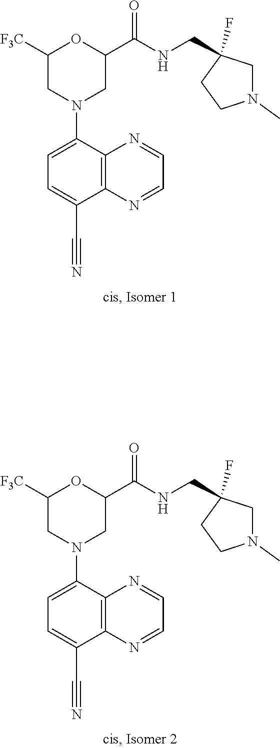 [SMILES: CN1CC[C@@](F)(CNC(=O)C2CN(c3ccc(C#N)c4nccnc34)CC(C(F)(F)F)O2)C1 ; CN1CC[C@@](F)(CNC(=O)C2CN(c3ccc(C#N)c4nccnc34)CC(C(F)(F)F)O2)C1]

The title compounds were prepared from cis-4-(8-cyanoquinoxalin-5-yl)-N-{[(3S)-3-fluoropyrrolidin-3-yl]methyl}-6-(trifluoromethyl)morpholine-2-carboxamide and Paraformaldehyde. The two diastereomeric products were obtained by separation on chiral-HPLC under the following conditions: column, CHIRALPAK IG-3, 0.46×5 cm, 3 um; mobile phase, hexane (with 0.1% DEA) in EtOH, 50% isocratic in 20 min; detector, UV 254 nm.

Isomer 1: MS: 467 [M+H]⁺. ¹H NMR (400 MHz, DMSO-d₆, ppm) δ 9.10 (d, J=1.8 Hz, 1H), 9.07 (s, 1H), 9.03 (d, J=1.8 Hz, 1H), 8.30 (d, J=8.3 Hz, 1H), 8.06 (t, J=6.2 Hz, 1H), 7.39 (d, J=8.4 Hz, 1H), 4.71 (s, 2H), 4.56 (dd, J=10.9, 2.7 Hz, 1H), 4.38 (dd, J=18.8, 12.3 Hz, 3H), 3.55-3.43 (m, 2H), 3.18 (dt, J=33.6, 11.6 Hz, 2H), 2.60 (dt, J=35.1, 10.5 Hz, 4H), 2.38 (q, J=7.6 Hz, 1H), 2.22 (s, 3H), 2.21 (s, 1H), 1.95 (dddd, J=49.2, 27.5, 13.7, 6.7 Hz, 2H).

Isomer 2: MS: 467 [M+H]⁺. ¹H NMR (400 MHz, DMSO-d₆, ppm) δ 9.13-8.99 (m, 2H), 8.33-8.26 (m, 1H), 8.10-8.02 (m, 1H), 7.39 (d, J=8.4 Hz, 1H), 4.73-4.69 (m, 1H), 4.60-4.52 (m, 1H), 4.44-4.32 (m, 2H), 3.55-3.43 (m, 2H), 3.28-3.09 (m, 2H), 2.71-2.51 (m, 3H), 2.43-2.33 (m, 1H), 2.22 (s, 3H), 2.10-1.80 (m, 2H).

### Example 357 (Isomer 1): (2R,6S)-4-(8-cyanoquinoxalin-5-yl)-N—(((S)-3-fluoro-1-methylpyrrolidin-3-yl)methyl)-6-(trifluoromethyl)morpholine-2-carboxamide & Example 358 (Isomer 2): (2S,6R)-4-(8-cyanoquinoxalin-5-yl)-N—(((S)-3-fluoro-1-methylpyrrolidin-3-yl)methyl)-6-(trifluoromethyl)morpholine-2-carboxamide

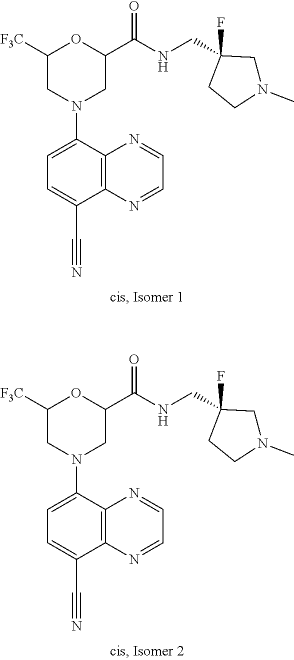 [SMILES: CN1CC[C@](F)(CNC(=O)C2CN(c3ccc(C#N)c4nccnc34)CC(C(F)(F)F)O2)C1 ; CN1CC[C@](F)(CNC(=O)C2CN(c3ccc(C#N)c4nccnc34)CC(C(F)(F)F)O2)C1]

The title compounds were prepared from cis-4-(8-cyanoquinoxalin-5-yl)-N-{[(3R)-3-fluoropyrrolidin-3-yl]methyl}-6-(trifluoromethyl)morpholine-2-carboxamide and Paraformaldehyde. The two diastereomeric products were obtained by separation on chiral-HPLC under the following conditions: column CHIRALPAK IA, 0.46×10 cm, 3 um; mobile phase, MtBE (with 0.1% DEA) in EtOH, 50% isocratic in 20 min; detector, UV 254 nm.

Isomer 1: MS: 467 [M+H]⁺. ¹H NMR (300 MHz, DMSO-d₆, ppm) δ 9.10 (d, J=1.8 Hz, 1H), 9.04 (d, J=1.8 Hz, 1H), 8.40-8.28 (m, 2H), 7.40 (d, J=8.4 Hz, 1H), 4.80-4.72 (m, 1H), 4.61-4.36 (m, 3H), 3.70-3.49 (m, 2H), 3.43-3.14 (m, 6H), 2.69 (s, 3H), 2.45-2.01 (m, 2H).

Isomer 2: MS: 467 [M+H]⁺. ¹H NMR (300 MHz, DMSO-d₆, ppm) δ 9.12 (d, J=1.8 Hz, 1H), 9.05 (d, J=1.8 Hz, 1H), 8.37-8.25 (m, 2H), 7.42 (d, J=8.4 Hz, 1H), 4.76-4.70 (m, 1H), 4.64-4.54 (m, 1H), 4.47-4.33 (m, 2H), 3.69-3.51 (m, 2H), 3.30-3.09 (m, 6H), 2.71 (s, 3H), 2.44-2.02 (m, 2H).

### Example 359 (Isomer 1): (2R,6S)-4-(8-cyanoquinoxalin-5-yl)-N-((4-fluoro-1-methylpiperidin-4-yl)methyl)-6-(trifluoromethyl)morpholine-2-carboxamide & Example 360 (Isomer 2): (2S,6R)-4-(8-cyanoquinoxalin-5-yl)-N-((4-fluoro-1-methylpiperidin-4-yl)methyl)-6-(trifluoromethyl)morpholine-2-carboxamide

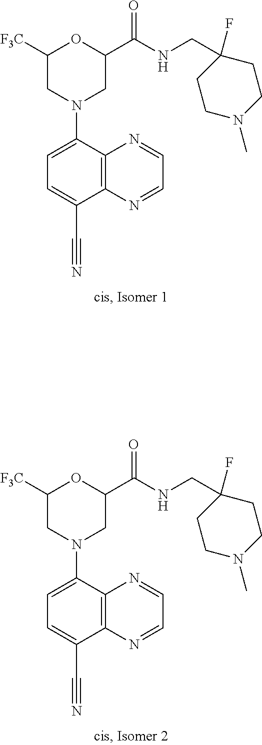 [SMILES: CN1CCC(F)(CNC(=O)C2CN(c3ccc(C#N)c4nccnc34)CC(C(F)(F)F)O2)CC1 ; CN1CCC(F)(CNC(=O)C2CN(c3ccc(C#N)c4nccnc34)CC(C(F)(F)F)O2)CC1]

The title compounds were prepared from cis-4-(8-cyanoquinoxalin-5-yl)-N-[(4-fluoropiperidin-4-yl)methyl]-6-(trifluoromethyl)morpholine-2-carboxamide and Paraformaldehyde. The two enantiomeric products were obtained by separation on chiral-HPLC under the following conditions: column CHIRALPAK IC-3, 0.46×5 cm, 3 um; mobile phase, MtBE (with 0.1% DEA) in EtOH, 70% isocratic in 20 min; detector, UV 254 nm.

Isomer 1: MS: 481 [M+H]⁺. ¹H NMR (400 MHz, DMSO-d₆, ppm) δ 9.06 (d, J=1.8 Hz, 1H), 9.02 (d, J=1.8 Hz, 1H), 8.30 (d, J=8.3 Hz, 1H), 7.96 (m, J=6.3 Hz, 1H), 7.39 (d, J=8.4 Hz, 1H), 4.70 (m, 1H), 4.57 (m, 1H), 4.37 (m, 2H), 3.46-3.33 (m, 2H), 3.27-3.09 (m, 2H), 2.55 (m, 2H), 2.17 (s, 3H), 2.11 (m, 2H), 1.68 (m, J=12.5, 3.2 Hz, 3H), 1.61-1.56 (m, 1H).

Isomer 2: MS: 481 [M+H]⁺. ¹H NMR (400 MHz, DMSO-d₆, ppm) δ 9.10 (d, J=1.8 Hz, 1H), 9.03 (d, J=1.8 Hz, 1H), 8.30 (d, J=8.3 Hz, 1H), 7.96 (t, J=6.3 Hz, 1H), 7.39 (d, J=8.4 Hz, 1H), 4.75-4.66 (m, 1H), 4.61-4.53 (m, 1H), 4.44-4.32 (m, 2H), 3.42-3.32 (m, 2H), 3.29-3.11 (m, 2H), 2.58-2.50 (m, 2H), 2.16 (s, 3H), 2.15-2.06 (m, 2H), 1.78-1.50 (m, 4H).

### Example 361 (Isomer 1): 8-[(2S,6S)-2-[(3-hydroxyazetidin-1-yl)methyl]-6-(trifluoromethyl)morpholin-4-yl]quinoxaline-5-carbonitrile & Example 362 (Isomer 2): 8-[(2R,6R)-2-[(3-hydroxyazetidin-1-yl)methyl]-6-(trifluoromethyl)morpholin-4-yl]quinoxaline-5-carbonitrile

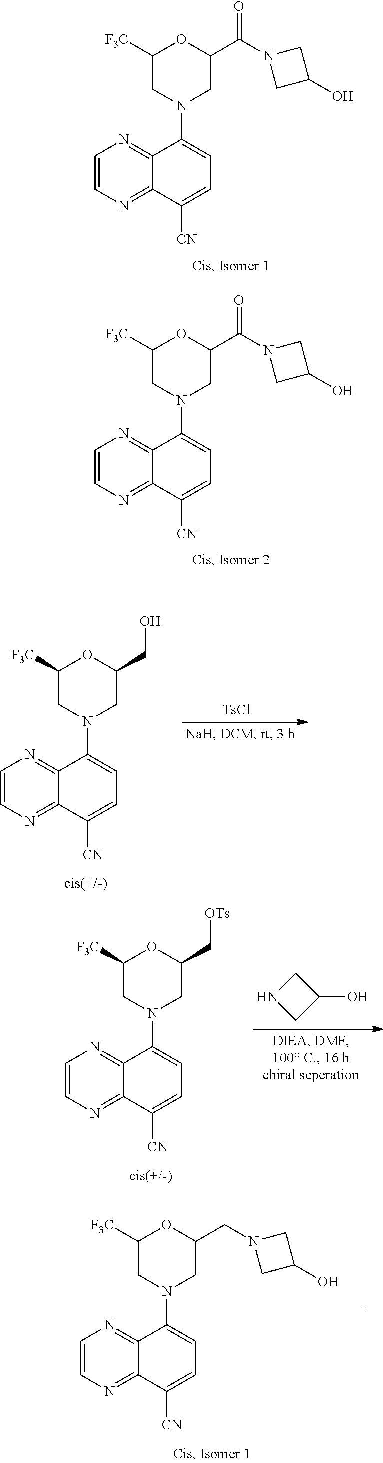

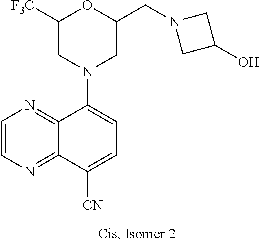 [SMILES: N#Cc1ccc(N2CC(CN3CC(O)C3)OC(C(F)(F)F)C2)c2nccnc12]

(cis-4-(8-cyanoquinoxalin-5-yl)-6-(trifluoromethyl)morpholin-2-yl)methyl 4-methylbenzenesulfonate: At 0° C., to a solution of 8-[cis-2-(hydroxymethyl)-6-(trifluoromethyl)morpholin-4-yl]quinoxaline-5-carbonitrile (94 mg, 0.28 mmol) in dichloromethane (20 mL) was added sodium hydride (63 mg, 2.66 mmol) in several batches. The resulting mixture was stirred for 30 min at 0° C., and then was added by TsCl (120 mg, 0.63 mmol) slowly. The resulting mixture was stirred for 3 h at room temperature. When the reaction was done, it was quenched by the addition of water (20 mL). The resulting mixture was extracted with ethyl acetate (30 mL×3). The organic phases were combined, washed with brine and dried over Na₂SO₄. The solvent was removed under reduced pressure and the residue was purified by flash chromatography eluting with ethyl acetate in hexane (0% to 15% gradient) to yield the title as a yellow solid (99 mg, 72%). MS: 493 [M+H]⁺.

8-[(2S,6S)-2-[(3-hydroxyazetidin-1-yl)methyl]-6-(trifluoromethyl)morpholin-4-yl]quinoxaline-5-carbonitrile & 8-[(2R,6R)-2-[(3-hydroxyazetidin-1-yl)methyl]-6-(trifluoromethyl)morpholin-4-yl]quinoxaline-5-carbonitrile: To a solution of cis-4-(8-cyanoquinoxalin-5-yl)-6-(trifluoromethyl)morpholin-2-yl 4-methylbenzene-1-sulfonate (80 mg, 0.16 mmol) in DMF (5 mL) was added azetidin-3-ol (34 mg, 0.46 mmol), DIEA (60 mg, 0.46 mmol). The resulting mixture was stirred for 16 h at 100° C. When the reaction was done, it was quenched by the addition of water (20 mL). The resulting mixture was extracted with ethyl acetate (50 mL×3). The organic phases were combined, washed with brine and dried over Na₂SO₄. The solvent was removed under reduced pressure and the residue was purified by prep-HPLC under the following conditions: column, XBridge Prep C18 OBD Column, 150×19 mm, 5 um; mobile phase, acetonitrile in water (with 10 mmol/L NH₄HCO₃ and 0.1% NH₃.H₂O), 38% to 70% gradient in 8 min; detector, UV 254 nm. Then the two enantiomeric products were obtained by separation on chiral-HPLC under the following conditions: column ChiralPAK IE-3, 0.46×5 cm, 3 um; mobile phase, hexane (with 20 mM NH₃H₂O) in EtOH, 75% isocratic in 20 min; detector, UV 220 nm.

Isomer 1: MS: 394 [M+H]⁺. ¹H NMR (400 MHz, Methanol-d₄, ppm) δ 8.99 (d, J=1.8 Hz, 1H), 8.93 (d, J=1.8 Hz, 1H), 8.17 (d, J=8.2 Hz, 1H), 7.30 (d, J=8.3 Hz, 1H), 4.54-4.42 (m, 2H), 4.42-4.32 (m, 1H), 4.13-4.02 (m, 2H), 3.83-3.69 (m, 2H), 3.13-3.01 (m, 3H), 2.98-2.88 (m, 1H), 2.84-2.65 (m, 2H).

Isomer 2: MS: 394 [M+H]⁺. ¹H NMR (400 MHz, Methanol-d₄, ppm) δ 8.98 (d, J=1.8 Hz, 1H), 8.93 (d, J=1.8 Hz, 1H), 8.17 (d, J=8.3 Hz, 1H), 7.30 (d, J=8.3 Hz, 1H), 4.55-4.42 (m, 2H), 4.37 (m, 1H), 4.07 (m, 2H), 3.83-3.68 (m, 2H), 3.14-3.0 (m, 3H), 2.93 (dd, J=12.6, 11.0 Hz, 1H), 2.85-2.62 (m, 2H).

The following compounds were synthesized in an analogous manner.

### Example 363 (Isomer 1): 8-[(2S,6S)-2-([2-oxo-1,7-diazaspiro[3.5]nonan-7-yl]methyl)-6-(trifluoromethyl)morpholin-4-yl]quinoxaline-5-carbonitrile & Example 364 (Isomer 2): 8-[(2R,6R)-2-([2-oxo-1,7-diazaspiro[3.5]nonan-7-yl]methyl)-6-(trifluoromethyl)morpholin-4-yl]quinoxaline-5-carbonitrile

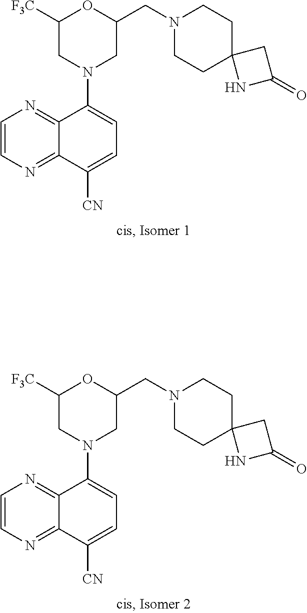 [SMILES: N#Cc1ccc(N2CC(CN3CCC4(CC3)CC(=O)N4)OC(C(F)(F)F)C2)c2nccnc12 ; N#Cc1ccc(N2CC(CN3CCC4(CC3)CC(=O)N4)OC(C(F)(F)F)C2)c2nccnc12]

The title compound was prepared from (cis-4-(8-cyanoquinoxalin-5-yl)-6-(trifluoromethyl)morpholin-2-yl)methyl 4-methylbenzenesulfonate and 1,7-diazaspiro[3.5]nonan-2-one. The two enantiomeric products were obtained by separation on chiral-HPLC under the following conditions: column, CHIRALPAK IG-3, 0.46×5 cm, 3 um; mobile phase, hexane/DCM (3:1, with 0.1% DEA) in EtOH, 50% isocratic in 20 min; detector, UV 254 nm.

Isomer 1: MS: 461 [M+H]⁺. ¹H NMR (400 MHz, Methanol-d₄, ppm) δ 9.0 (d, J=1.8 Hz, 1H), 8.93 (d, J=1.8 Hz, 1H), 8.18 (d, J=8.3 Hz, 1H), 7.32 (d, J=8.3 Hz, 1H), 4.53 (d, J=8.0 Hz, 1H), 4.48-4.40 (m, 1H), 4.28 (s, 1H), 4.20 (m, J=12.3, 2.2 Hz, 1H), 3.12 (dd, J=11.9, 10.7 Hz, 1H), 2.93 (dd, J=12.3, 10.5 Hz, 2H), 2.73 (d, J=12.0 Hz, 7H), 1.90 (m, 4H).

Isomer 2: MS: 461 [M+H]⁺. ¹H NMR (400 MHz, Methanol-d₄, ppm) δ 9.03-8.98 (m, 1H), 8.96-8.91 (m, 1H), 8.23-8.15 (m, 1H), 7.36-7.29 (m, 1H), 4.62-4.55 (m, 1H), 4.48-4.40 (m, 1H), 4.37-4.33 (m, 1H), 4.24-4.16 (m, 1H), 3.19-3.09 (m, 2H), 3.08-2.67 (m, 8H), 1.99-1.94 (m, 4H).

### Example 365: 1-[(2R,6R)-4-(8-Cyano-[1,7]naphthyridin-5-yl)-6-methyl-morpholin-2-ylmethyl]-1H-pyrazole-4-carboxylic acid

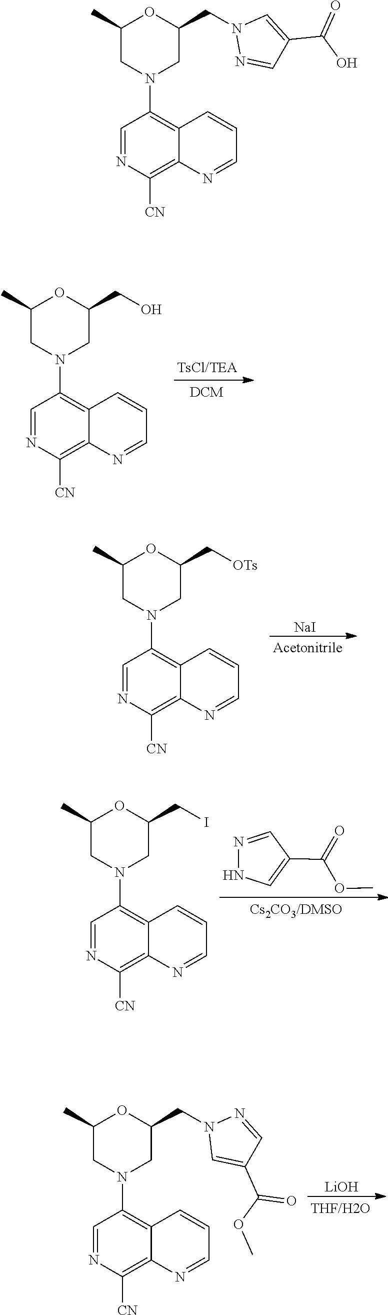

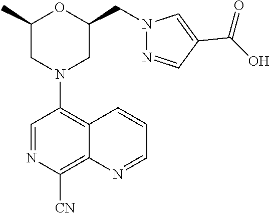 [SMILES: C[C@@H]1CN(c2cnc(C#N)c3ncccc23)C[C@H](Cn2cc(C(=O)O)cn2)O1]

Toluene-4-sulfonic acid (2R,6R)-4-(8-cyano-[1,7]naphthyridin-5-yl)-6-methyl-morpholin-2-ylmethyl ester: To a stirred solution of 5-((2R,6R)-2-Hydroxymethyl-6-methyl-morpholin-4-yl)-[1,7]naphthyridine-8-carbonitrile (933 mg; 3.28 mmol; 1.0 eq.) in DCM (2.0 ml) was added p-toluenesulfonyl chloride (750 mg; 3.94 mmol; 1.20 eq.), followed by TEA (0.91 ml; 6.56 mmol; 2.0 eq.), The mixture was stirred at RT for 4 hr, until the reaction was completed. The reaction mixture was diluted with EA (100 ml), washed with brine. The organic layer was dried over Na₂SO₄ and concentrated to provide the title compound as a yellow solid (1486 mg, quantities yield), which was directly used for the next step reaction without purification. MS: 439 [M+H]⁺.

5-((2R,6R)-2-Iodomethyl-6-methyl-morpholin-4-yl)-[1,7] naphthyridine-8-carbonitrile: Into a 25-mL microwave vial was placed Toluene-4-sulfonic acid (2R,6R)-4-(8-cyano-[1,7]naphthyridin-5-yl)-6-methyl-morpholin-2-ylmethyl ester (1442 mg; 3.29 mmol; 1.0 eq.), sodium iodide (2464 mg; 16.44 mmol; 5.0 eq.) and acetonitrile (15 ml). The sealed vial was stirred at 80° C. overnight. The completed reaction was diluted with EA (100 ml), washed with 15 mL aqueous NaHSO₃ (10%) solution, and then with NaHCO₃ (5%) and brine. The organic phase was dried over Na₂SO₄ and concentrated to give the title compound as a yellow solid (1300 mg), which was directly carried to the next step reaction without purification. MS: 395 [M+H]⁺.

1-[(2R,6R)-4-(8-Cyano-[1,7]naphthyridin-5-yl)-6-methyl-morpholin-2-ylmethyl]-1H-pyrazole-4-carboxylic acid methyl ester: In a 10 ml microwave tube was placed 5-((2R,6R)-2-Iodomethyl-6-methyl-morpholin-4-yl)-[1,7]naphthyridine-8-carbonitrile (70 mg; 0.18 mmol; 1.0 eq.), cesium carbonate (115 mg; 0.36 mmol; 2.0 eq.), 1H-Pyrazole-4-carboxylic acid methyl ester (34 mg; 0.27 mmol; 1.50 eq.) and DMSO (1 ml). The sealed tube was stirred at 80° C. for 3 hr, until the reaction was completed. The crude was purified by prep HPLC with mobile phase: 20-60% ACN/water (contained 0.1% ammonia) to yield the title compound (70 mg, 43%). MS: 393 [M+H]⁺.

1-[(2R,6R)-4-(8-Cyano-[1,7]naphthyridin-5-yl)-6-methyl-morpholin-2-ylmethyl]-1H-pyrazole-4-carboxylic acid: A mixture of 1-[(2R,6R)-4-(8-Cyano-[1,7]naphthyridin-5-yl)-6-methyl-morpholin-2-ylmethyl]-1H-pyrazole-4-carboxylic acid methyl ester (15 mg; 0.04 mmol; 1.0 eq.), lithium hydroxide hydrate (4 mg; 0.08 mmol; 2.0 eq.) in water (1 ml) and THF (1 ml) was stirred at RT for 3 hr, until the reaction was completed. The solvent was removed. To the residue was added DCM (1 ml) and TFA (1 ml). The resulting mixture was stirred at RT for 30 min, until the reaction was completed. The crude was purified by prep HPLC, mobile phase 10-60% ACN/water (contained 0.1% formic acid) to yield the title compound (12 mg, yield: 81%). MS: 379 [M+H]⁺. ¹H NMR (400 MHz, DMSO-d₆) δ 9.06 (d, J=2.0 Hz, 1H), 8.94 (s, 1H), 8.76 (d, J=1.6 Hz, 1H), 8.26 (d, J=8.3 Hz, 1H), 7.26 (d, J=8.4 Hz, 1H), 4.76-4.54 (m, 2H), 4.37-4.21 (m, 2H), 4.15 (d, J=12.4 Hz, 1H), 3.85 (s, 3H), 2.80 (dt, J=37.9, 11.4 Hz, 2H), 1.14 (d, J=6.2 Hz, 3H).

### Example 366: 5-((2R,6S)-2-Methyl-6-piperazin-1-ylmethyl-morpholin-4-yl)-[1,7]naphthyridine-8-carbonitrile

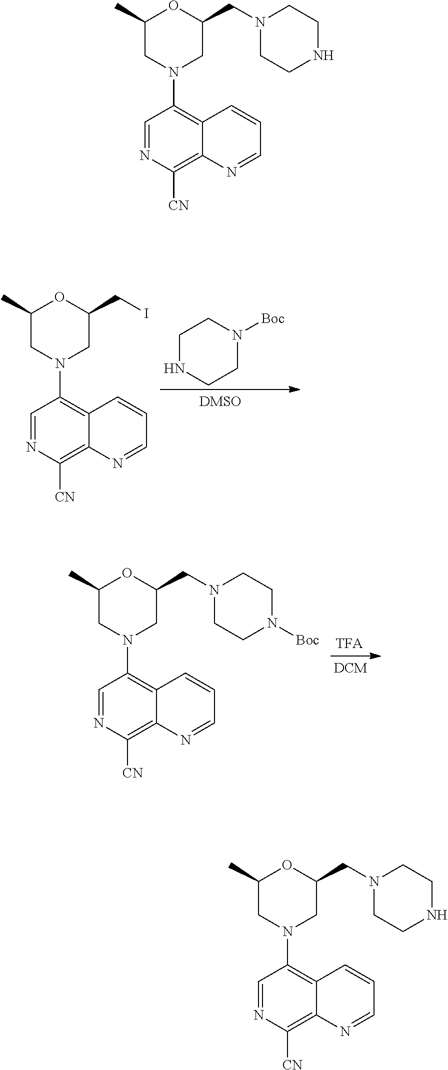

4-[(2S,6R)-4-(8-Cyano-[1,7]naphthyridin-5-yl)-6-methyl-morpholin-2-ylmethyl]-piperazine-1-carboxylic acid tert-butyl ester: A mixture of 5-((2R,6R)-2-Iodomethyl-6-methyl-morpholin-4-yl)-[1,7]naphthyridine-8-carbonitrile (382 mg; 0.97 mmol; 1.0 eq.), Piperazine-1-carboxylic acid tert-butyl ester (902 mg; 4.85 mmol; 5.0 eq.) and DMSO (3 ml) in 25 ml vial were stirred at 60° C. for 6 hr, until the reaction was completed. The reaction mixture was diluted with EA (80 ml) and water (30 ml). The organic layer was washed with brine, dried over Na₂SO₄, and concentrated to yield the compound, which directly was used for the next step reaction. MS: 453 [M+H]⁺.

5-((2R,6S)-2-Methyl-6-piperazin-1-ylmethyl-morpholin-4-yl)-[1,7]naphthyridine-8-carbonitrile: To a solution of 4-[(2S,6R)-4-(8-Cyano-[1,7]naphthyridin-5-yl)-6-methyl-morpholin-2-ylmethyl]-piperazine-1-carboxylic acid tert-butyl ester (440 mg; 0.97 mmol; 1.0 eq) in DCM (2 ml) was added 2 ml of TFA. The resulting mixture was stirred at RT for 1 hr, until the reaction was completed. The reaction mixture was diluted with DCM, washed with 10% Na₂CO₃ (aq), then brine. The organic layer was dried over Na₂SO₄ and concentrated. The residue was purified by prep HPLC with Mobile phase: 10-60% ACN/water (contained 0.1% ammonia), to yield the title compound (300 mg). MS: 351 [M+H]⁺. ¹H NMR (400 MHz, DMSO-d6) 9.19 (d, J=4.1 Hz, 1H), 8.58 (d, J=8.6 Hz, 1H), 8.38 (s, 1H), 7.88 (dd, J=8.7, 4.2 Hz, 1H), 4.11-3.91 (m, 2H), 3.54 (dd, J=22.6, 12.1 Hz, 2H), 2.77 (q, J=10.9 Hz, 2H), 2.65 (d, J=5.0 Hz, 3H), 2.46-2.19 (m, 6H), 1.17 (d, J=6.1 Hz, 3H).

### Example 367: 5-[(2R,6S)-2-Methyl-6-(4-morpholin-4-yl-piperidin-1-ylmethyl)-morpholin-4-yl]-[1,7]naphthyridine-8-carbonitrile

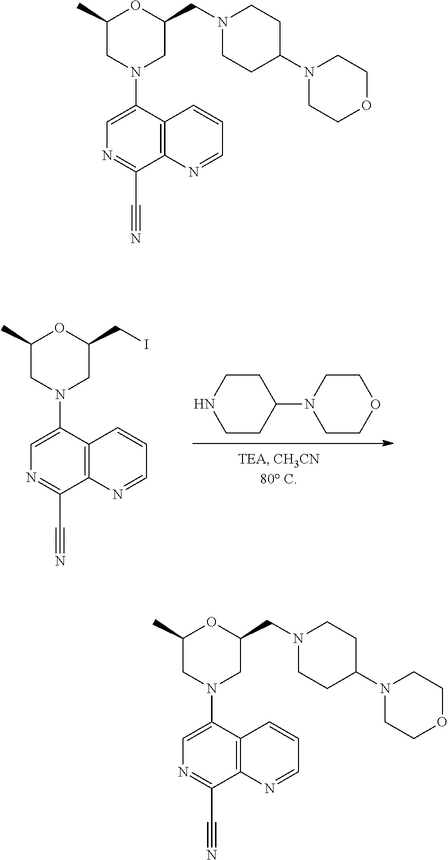 [SMILES: C[C@@H]1CN(c2cnc(C#N)c3ncccc23)C[C@H](CN2CCC(N3CCOCC3)CC2)O1 ; C[C@@H]1CN(c2cnc(C#N)c3ncccc23)C[C@H](CI)O1 ; C1CC(N2CCOCC2)CCN1 ; C[C@@H]1CN(c2cnc(C#N)c3ncccc23)C[C@H](CN2CCC(N3CCOCC3)CC2)O1]

Into a 25 mL vial was placed 5-((2R,6R)-2-Iodomethyl-6-methyl-morpholin-4-yl)-[1,7]naphthyridine-8-carbonitrile (50.0 mg; 0.127 mmol; 1.0 eq.), 4-(piperidin-4-yl)morpholine (43.2 mg; 0.254 mmol; 2.0 eq.), MeCN (2.0 ml) and TEA (55.2 μl; 0.397 mmol; 3.13 eq.). The reaction solution was stirred for 10 h at 80° C. LCMS showed that the reaction was complete. 3 mL of DMSO were added and the resulting solution filtered with a Pall acrodisc 0.45 uM. The product was purified on reverse phase system using a gradient of 05-60% CH₃CN/H₂O (0.1% Ammonium Hydroxide) in 2 injections of 1 mL each. The desired fractions were evaporated to provide the title compound (8.0 mg; 15%) as a yellow solid. MS: 437 [M+H]⁺. ¹H NMR (400 MHz, Methanol-d₄) δ 9.19-9.07 (m, 1H), 8.63 (d, J=8.7 Hz, 1H), 8.34 (s, 1H), 7.83 (dt, J=6.7, 3.0 Hz, 1H), 4.21-3.97 (m, 2H), 3.72 (t, J=4.1 Hz, 4H), 3.53 (dd, J=16.5, 12.5 Hz, 2H), 3.20 (d, J=12.0 Hz, 1H), 3.05 (d, J=11.8 Hz, 1H), 2.88-2.72 (m, 2H), 2.67-2.37 (m, 6H), 2.30-2.07 (m, 3H), 1.93 (ddd, J=13.6, 6.6, 3.2 Hz, 2H), 1.69-1.50 (m, 2H), 1.27 (dd, J=6.4, 2.1 Hz, 3H).

The following compounds were synthesized in an analogous manner.

### Example 368: 5-[(2R,6S)-2-Methyl-6-(4-methyl-piperazin-1-ylmethyl)-morpholin-4-yl]-naphthyridine-8-carbonitrile

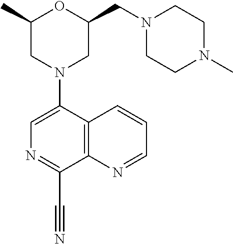

The title compound was prepared from 5-((2R,6R)-2-Iodomethyl-6-methyl-morpholin-4-yl)-[1,7]naphthyridine-8-carbonitrile and 1-methylpiperazine. MS: 367 [M+H]⁺. ¹H NMR (400 MHz, Methanol-d₄) δ 9.12 (s, 1H), 8.63 (d, J=8.7 Hz, 1H), 8.34 (s, 1H), 7.92-7.73 (m, 1H), 4.20-3.99 (m, 2H), 3.53 (dd, J=16.6, 12.5 Hz, 2H), 3.03-2.35 (m, 12H), 2.30 (s, 3H), 1.27 (d, J=5.8 Hz, 3H).

### Example 369: 5-[(2S,6R)-2-(4-Amino-3,3-difluoro-piperidin-1-ylmethyl)-6-methyl-morpholin-4-yl]-[1,7]naphthyridine-8-carbonitrile

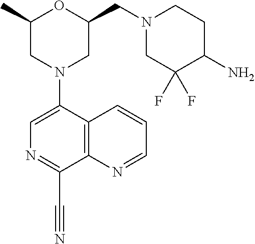 [SMILES: C[C@@H]1CN(c2cnc(C#N)c3ncccc23)C[C@H](CN2CCC(N)C(F)(F)C2)O1]

The title compound was prepared from 5-((2R,6R)-2-Iodomethyl-6-methyl-morpholin-4-yl)-[1,7]naphthyridine-8-carbonitrile and 3,3-difluoropiperidin-4-amine. MS: 403 [M+H]⁺. ¹H NMR (400 MHz, Methanol-d₄) δ 9.13 (s, 1H), 8.64 (d, J=8.7 Hz, 1H), 8.34 (s, 1H), 7.83 (d, J=8.9 Hz, 1H), 4.27-3.98 (m, 2H), 3.75-3.41 (m, 3H), 3.09-2.73 (m, 4H), 2.65 (dt, J=22.2, 14.4 Hz, 2H), 2.43 (tt, J=29.0, 16.2 Hz, 2H), 1.95-1.82 (m, 1H), 1.67-1.53 (m, 1H), 1.28 (d, J=6.4 Hz, 3H).

### Example 370: 5-[(2S,6R)-2-(4-Aminomethyl-4-fluoro-piperidin-1-ylmethyl)-6-methyl-morpholin-4-yl]-[1,7]naphthyridine-8-carbonitrile

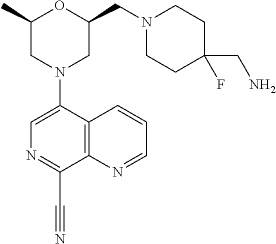 [SMILES: C[C@@H]1CN(c2cnc(C#N)c3ncccc23)C[C@H](CN2CCC(F)(CN)CC2)O1]

The title compound was prepared from 5-((2R,6R)-2-Iodomethyl-6-methyl-morpholin-4-yl)-[1,7]naphthyridine-8-carbonitrile and C-(4-Fluoro-piperidin-4-yl)-methylamine trifluoroacetate. MS: 399 [M+H]⁺. ¹H NMR (400 MHz, Methanol-d₄) δ 9.12 (s, 1H), 8.63 (d, J=8.7 Hz, 1H), 8.35 (s, 1H), 7.82 (dt, J=7.3, 3.0 Hz, 1H), 4.29-3.97 (m, 2H), 3.54 (t, J=14.0 Hz, 2H), 3.03-2.84 (m, 3H), 2.78 (dd, J=21.1, 9.1 Hz, 4H), 2.64-2.38 (m, 3H), 1.89 (t, J=12.4 Hz, 2H), 1.82-1.58 (m, 2H), 1.28 (dd, J=6.3, 2.3 Hz, 3H).

### Example 371: 5-[(2S,6R)-2-(4-Ethyl-piperazin-1-ylmethyl)-6-methyl-morpholin-4-yl]-naphthyridine-8-carbonitrile

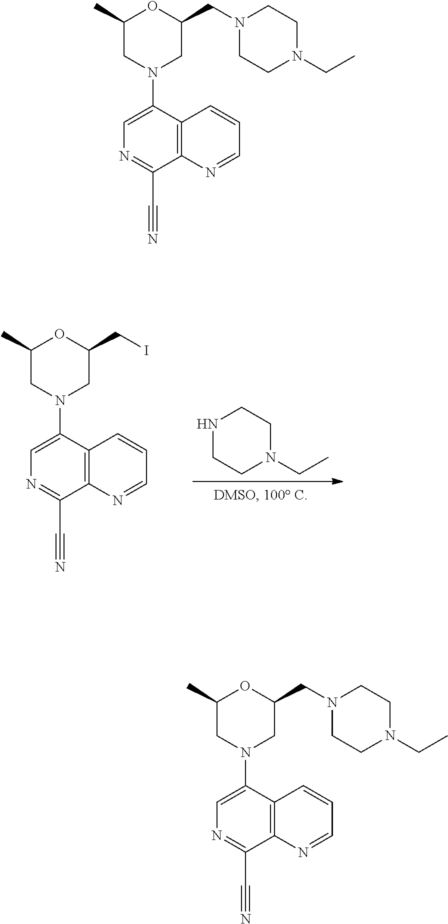

5-((2R,6R)-2-Iodomethyl-6-methyl-morpholin-4-yl)-[1,7]naphthyridine-8-carbonitrile (70.0 mg; 0.18 mmol; 1.0 eq.) and 1-Ethyl-piperazine (101.38 mg; 0.89 mmol; 5.0 eq.) in DMSO (1 mL) was stirred at 100° C. overnight. Once complete, the reaction was purified by prep HPLC with an acetonitrile/water (0.1% NH₄OH modified) gradient to afford the title compound (16.30 mg; 0.04 mmol; 24.1%). MS: 381.5 [M+H]⁺. 1H NMR (400 MHz, DMSO-d₆) δ 9.19 (dd, J=4.1, 1.6 Hz, 1H), 8.58 (dd, J=8.7, 1.6 Hz, 1H), 8.37 (s, 1H), 7.87 (dd, J=8.7, 4.1 Hz, 1H), 4.08-3.91 (m, 2H), 3.58-3.47 (m, 2H), 2.77 (q, J=11.0 Hz, 2H), 2.41 (dd, J=6.0, 2.1 Hz, 4H), 2.34 (s, 3H), 2.27 (q, J=7.2 Hz, 4H), 1.17 (d, J=6.2 Hz, 3H), 0.97 (t, J=7.2 Hz, 3H).

The following examples were prepared in an analogous manner.

### Example 372: 5-{(2S,6R)-2-[4-(2-Hydroxy-ethyl)-piperazin-1-ylmethyl]-6-methyl-morpholin-4-yl}-[1,7]naphthyridine-8-carbonitrile

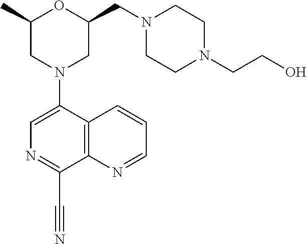 [SMILES: C[C@@H]1CN(c2cnc(C#N)c3ncccc23)C[C@H](CN2CCN(CCO)CC2)O1]

The title compound was prepared from 5-((2R,6R)-2-Iodomethyl-6-methyl-morpholin-4-yl)-[1,7]naphthyridine-8-carbonitrile and 2-Piperazin-1-yl-ethanol. MS: 397 [M+H]⁺. 1H NMR (400 MHz, DMSO-d₆) δ 9.19 (d, J=4.1 Hz, 1H), 8.58 (d, J=8.8 Hz, 1H), 8.37 (s, 1H), 7.87 (dd, J=8.7, 4.1 Hz, 1H), 4.33 (t, J=5.5 Hz, 1H), 3.54 (d, J=14.3 Hz, 3H), 3.47 (q, J=6.2 Hz, 4H), 2.40 (s, 5H), 2.34 (t, J=6.2 Hz, 3H), 1.17 (d, J=6.3 Hz, 3H).

### Example 373: 5-{(2S,6R)-2-[(3-Fluoro-2-hydroxy-propylamino)-methyl]-6-methyl-morpholin-4-yl}-[1,7]naphthyridine-8-carbonitrile

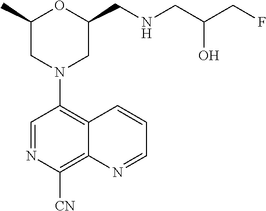 [SMILES: C[C@@H]1CN(c2cnc(C#N)c3ncccc23)C[C@H](CNCC(O)CF)O1]

The title compound was prepared from 5-((2R,6R)-2-Iodomethyl-6-methyl-morpholin-4-yl)-[1,7]naphthyridine-8-carbonitrile and 1-Amino-3-fluoro-propan-2-ol. MS: 360 [M+H]⁺. ¹H NMR (400 MHz, Methanol-d₄) δ 9.12 (d, J=3.6 Hz, 1H), 8.65 (d, J=8.7 Hz, 1H), 8.36 (s, 1H), 7.83 (dt, J=8.1, 3.5 Hz, 1H), 4.45 (d, J=8.7 Hz, 1H), 4.33 (d, J=8.9 Hz, 1H), 4.20-4.04 (m, 2H), 3.95 (d, J=18.8 Hz, 1H), 3.52 (d, J=10.6 Hz, 2H), 2.99-2.58 (m, 6H), 1.28 (d, J=6.2 Hz, 3H).

### Example 374: N-{2-[(2R,6R)-4-(8-Cyano-[1,7]naphthyridin-5-yl)-6-methyl-morpholin-2-ylmethylsulfanyl]-ethyl}-acetamide

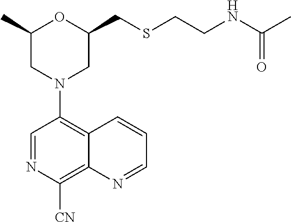 [SMILES: CC(=O)NCCSC[C@H]1CN(c2cnc(C#N)c3ncccc23)C[C@@H](C)O1]

The title compound was prepared from 5-((2R,6R)-2-Iodomethyl-6-methyl-morpholin-4-yl)-[1,7]naphthyridine-8-carbonitrile and N-(2-Mercapto-ethyl)-acetamide. MS: 386 [M+H]⁺. ¹H NMR (400 MHz, DMSO-d₆) δ 9.19 (dd, J=4.1, 1.7 Hz, 1H), 8.60 (dd, J=8.6, 1.7 Hz, 1H), 8.39 (d, J=1.5 Hz, 1H), 8.04-7.82 (m, 2H), 3.99 (d, J=8.8 Hz, 2H), 3.62 (d, J=12.1 Hz, 1H), 3.52 (dd, J=12.0, 2.0 Hz, 1H), 3.22 (t, J=6.7 Hz, 2H), 2.81 (dt, J=31.9, 11.3 Hz, 2H), 2.66 (dt, J=21.0, 6.6 Hz, 3H), 1.80 (d, J=1.4 Hz, 3H), 1.18 (d, J=6.1 Hz, 3H).

### Example 375: N-{2-[(2R,6R)-4-(8-Cyano-[1,7]naphthyridin-5-yl)-6-methyl-morpholin-2-ylmethylsulfanyl]-ethyl}-acetamide

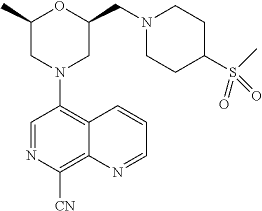 [SMILES: C[C@@H]1CN(c2cnc(C#N)c3ncccc23)C[C@H](CN2CCC(S(C)(=O)=O)CC2)O1]

The title compound prepared from 5-((2R,6R)-2-Iodomethyl-6-methyl-morpholin-4-yl)-[1,7]naphthyridine-8-carbonitrile and 4-Methanesulfonyl-piperidine. MS: 360 [M+H]⁺. ¹H NMR (400 MHz, DMSO-d₆) δ 9.18 (dd, J=3.9, 1.9 Hz, 1H), 8.59 (d, J=8.5 Hz, 1H), 8.38 (d, J=1.7 Hz, 1H), 7.95-7.79 (m, 1H), 4.14-3.85 (m, 2H), 3.53 (t, J=13.7 Hz, 2H), 3.13 (d, J=10.9 Hz, 1H), 3.01 (d, J=12.7 Hz, 2H), 2.91 (d, J=1.7 Hz, 3H), 2.77 (dt, J=16.8, 11.1 Hz, 2H), 2.46 (s, 2H), 2.15-1.85 (m, 4H), 1.68-1.43 (m, 2H), 1.17 (d, J=6.1 Hz, 3H).

### Example 376: 5-[(2S,6R)-2-(1,1-Dioxo-1lambda6-thiomorpholin-4-ylmethyl)-6-methyl-morpholin-4-yl]-[1,7]naphthyridine-8-carbonitrile

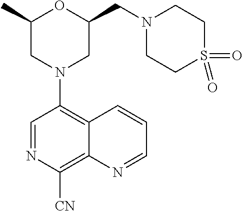 [SMILES: C[C@@H]1CN(c2cnc(C#N)c3ncccc23)C[C@H](CN2CCS(=O)(=O)CC2)O1]

The title compound was prep pared from 5-((2R,6R)-2-Iodomethyl-6-methyl-morpholin-4-yl)-[1,7]naphthyridine-8-carbonitrile and Thiomorpholine 1,1-dioxide. MS: 402 [M+H]⁺. ¹H NMR (400 MHz, DMSO-d₆) δ 9.18 (d, J=4.1 Hz, 1H), 8.66-8.52 (m, 1H), 8.39 (s, 1H), 7.87 (dd, J=8.8, 4.2 Hz, 1H), 4.0 (d, J=18.8 Hz, 2H), 3.64-3.44 (m, 2H), 3.03 (d, J=32.9 Hz, 6H), 2.91-2.60 (m, 4H), 1.16 (d, J=5.8 Hz, 3H).

### Example 377: 5-((2S,6R)-2-{[(4-Fluoro-tetrahydro-pyran-4-ylmethyl)-amino]-methyl}-6-methyl-morpholin-4-yl)-[1,7]naphthyridine-8-carbonitrile

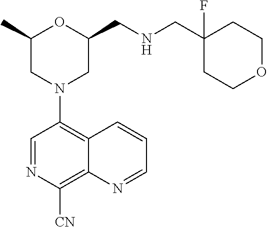 [SMILES: C[C@@H]1CN(c2cnc(C#N)c3ncccc23)C[C@H](CNCC2(F)CCOCC2)O1]

The title compound was prep pared from 5-((2R,6R)-2-Iodomethyl-6-methyl-morpholin-4-yl)-[1,7]naphthyridine-8-carbonitrile and (4-fluorotetrahydro-2 h-pyran-4-yl)methanamine. MS: 400 [M+H]⁺. ¹H NMR (400 MHz, DMSO-d₆) δ 9.25-9.13 (m, 1H), 8.60 (dt, J=8.7, 1.3 Hz, 1H), 8.38 (s, 1H), 7.86 (dd, J=8.7, 4.1 Hz, 1H), 3.96 (t, J=8.3 Hz, 2H), 3.78-3.42 (m, 6H), 2.90-2.56 (m, 6H), 1.79 (d, J=4.5 Hz, 1H), 1.78-1.59 (m, 4H), 1.18 (d, J=6.2 Hz, 3H).

### Example 378: 5-((2S,6R)-2-{[(3-Hydroxy-oxetan-3-ylmethyl)-amino]-methyl}-6-methyl-morpholin-4-yl)-[1,7]naphthyridine-8-carbonitrile

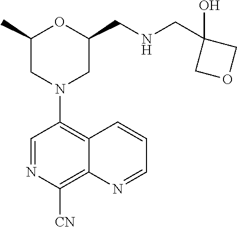 [SMILES: C[C@@H]1CN(c2cnc(C#N)c3ncccc23)C[C@H](CNCC2(O)COC2)O1]

The title compound was prep pared from 5-((2R,6R)-2-Iodomethyl-6-methyl-morpholin-4-yl)-[1,7]naphthyridine-8-carbonitrile and 3-Aminomethyl-oxetan-3-ol. MS: 370 [M+H]⁺. ¹H NMR (400 MHz, DMSO-d₆) δ 9.18 (dd, J=4.1, 1.5 Hz, 1H), 8.61 (dd, J=8.7, 1.5 Hz, 1H), 8.39 (s, 1H), 7.86 (dd, J=8.7, 4.1 Hz, 1H), 5.64 (s, 1H), 4.36 (p, J=6.2 Hz, 4H), 3.96 (dt, J=12.5, 5.9 Hz, 2H), 3.55 (dd, J=27.5, 12.1 Hz, 2H), 2.94-2.62 (m, 6H), 1.80 (s, 1H), 1.18 (d, J=6.2 Hz, 3H).

### Example 379: 5-[(2R,6S)-2-Methyl-6-(3-oxo-piperazin-1-ylmethyl)-morpholin-4-yl]-naphthyridine-8-carbonitrile

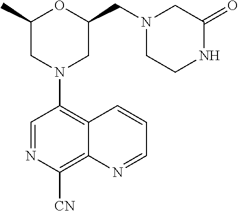 [SMILES: C[C@@H]1CN(c2cnc(C#N)c3ncccc23)C[C@H](CN2CCNC(=O)C2)O1]

The title compound was prep pared from 5-((2R,6R)-2-Iodomethyl-6-methyl-morpholin-4-yl)-[1,7]naphthyridine-8-carbonitrile and Piperazin-2-one. MS: 367 [M+H]⁺. ¹H NMR (400 MHz, DMSO-d₆) δ 9.18 (dt, J=4.0, 1.6 Hz, 1H), 8.66-8.55 (m, 1H), 8.38 (d, J=2.3 Hz, 1H), 7.86 (dd, J=8.7, 4.0 Hz, 1H), 7.69 (s, 1H), 4.07 (d, J=8.4 Hz, 1H), 3.97 (t, J=7.8 Hz, 1H), 3.52 (t, J=11.0 Hz, 2H), 3.20-3.08 (m, 2H), 3.04 (d, J=2.0 Hz, 2H), 2.87-2.66 (m, 3H), 2.66-2.53 (m, 1H), 1.18 (dd, J=6.3, 2.0 Hz, 3H).

### Example 380: N-(2-{[(2S,6R)-4-(8-Cyano-[1,7]naphthyridin-5-yl)-6-methyl-morpholin-2-ylmethyl]-amino}-ethyl)-acetamide

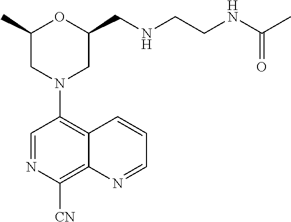 [SMILES: CC(=O)NCCNC[C@H]1CN(c2cnc(C#N)c3ncccc23)C[C@@H](C)O1]

The title compound was prep pared from 5-((2R,6R)-2-Iodomethyl-6-methyl-morpholin-4-yl)-[1,7]naphthyridine-8-carbonitrile and N-(2-Amino-ethyl)-acetamide. MS: 369 [M+H]⁺. ¹H NMR (400 MHz, Methanol-d₄) δ 9.18-9.03 (m, 1H), 8.63 (dd, J=8.7, 1.5 Hz, 1H), 8.34 (s, 1H), 7.82 (dd, J=8.7, 4.2 Hz, 1H), 4.08 (tdd, J=9.0, 5.4, 3.0 Hz, 2H), 3.54 (dd, J=4.8, 2.3 Hz, 2H), 3.35 (t, J=6.5 Hz, 2H), 2.95-2.70 (m, 6H), 1.97 (s, 3H), 1.29 (d, J=6.2 Hz, 3H).

### Example 381: 5-{(2S,6R)-2-[(1-Acetyl-piperidin-4-ylamino)-methyl]-6-methyl-morpholin-4-yl}-[1,7]naphthyridine-8-carbonitrile

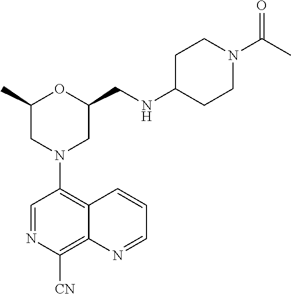 [SMILES: CC(=O)N1CCC(NC[C@H]2CN(c3cnc(C#N)c4ncccc34)C[C@@H](C)O2)CC1]

The title compound was prepared from 5-((2R,6R)-2-Iodomethyl-6-methyl-morpholin-4-yl)-[1,7]naphthyridine-8-carbonitrile and 1-acetylpiperidin-4-amine. MS: 409 [M+H]⁺. ¹H NMR (400 MHz, DMSO-d₆) δ 9.25-9.14 (m, 1H), 8.59 (d, J=8.7 Hz, 1H), 8.38 (d, J=1.3 Hz, 1H), 7.87 (ddd, J=8.6, 4.1, 1.2 Hz, 1H), 4.19-4.07 (m, 1H), 4.04-3.81 (m, 2H), 3.71 (d, J=13.7 Hz, 1H), 3.56 (dd, J=34.1, 12.1 Hz, 2H), 3.05 (t, J=12.4 Hz, 1H), 2.89-2.56 (m, 6H), 1.97 (d, J=1.2 Hz, 3H), 1.86-1.66 (m, 3H), 1.29-1.12 (m, 4H), 1.06 (q, J=11.0, 10.5 Hz, 1H).

### Example 382: 4-{[(2S,6R)-4-(8-cyano-1,7-naphthyridin-5-yl)-6-methylmorpholin-2-yl]methyl}piperazine-1-sulfonamide

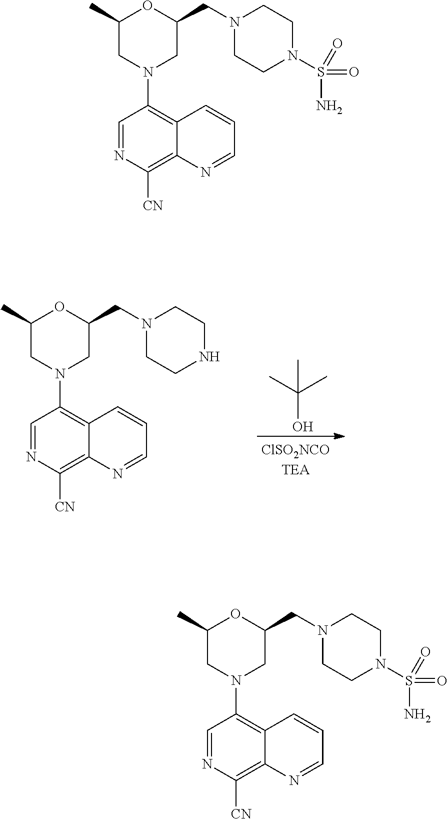

To a solution of 2-Methyl-propan-2-ol (23 mg; 0.31 mmol; 2.20 eq.) in 1 mL DCM was added chlorosulfonyl isocyanate (0.02 ml; 0.28 mmol; 2.0 eq.). The mixture was stirred for 2 hr at RT, then added 5-((2R,6S)-2-Methyl-6-piperazin-1-ylmethyl-morpholin-4-yl)-[1,7]naphthyridine-8-carbonitrile (50 mg; 0.14 mmol; 1.0 eq.) and triethylamine (0.06 ml; 0.43 mmol; 3.0 eq.). The resulted mixture was stirred at RT for 2 hr, until the reaction was completed. The reaction was quenched with 0.1 ml methanol, and then added 1 ml of TFA. The solution was stirred at RT for 1 hr. LCMS indicated the reaction finished. The solvent was removed. The residue was neutralized with TEA to PH>7 and purified by prep HPLC to provide the title compound (8 mg; 13%). MS: 360 [M+H]⁺. ¹H NMR (400 MHz, DMSO-d₆) δ 9.18 (d, J=4.0 Hz, 1H), 8.59 (dd, J=8.7, 1.7 Hz, 1H), 8.38 (d, J=1.4 Hz, 1H), 7.87 (dd, J=8.7, 4.2 Hz, 1H), 6.72 (s, 2H), 4.0 (dd, J=36.5, 7.6 Hz, 2H), 3.53 (t, J=14.1 Hz, 2H), 2.94 (t, J=5.0 Hz, 3H), 2.78 (dt, J=17.3, 11.2 Hz, 2H), 2.61 (d, J=7.8 Hz, 2H), 2.46-2.34 (m, 1H), 1.17 (d, J=6.1 Hz, 3H), 0.93 (td, J=7.2, 1.5 Hz, 1H).

### Example 383: [(2R,6R)-6-Methyl-4-(8-trifluoromethyl-[1,7]naphthyridin-5-yl)-morpholin-2-yl]-methanol

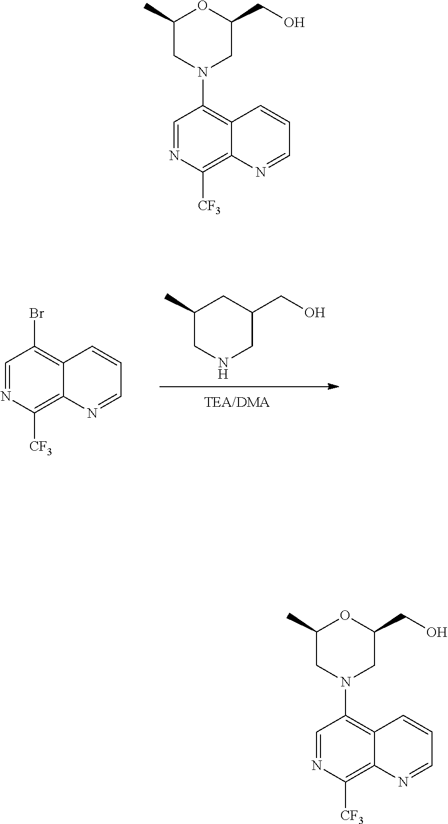

To a 20 ml microwave vial, was added 5-Bromo-8-trifluoromethyl-[1,7]naphthyridine (1200 mg; 4.21 mmol; 1.0 eq.), (2R,6R)-6-Methyl-morpholin-2-yl)-methanolhydrochloride (741 mg; 4.42 mmol; 1.05 eq.), TEA (1.89 ml; 10.53 mmol; 2.50 eq.) and DMA (5.7 ml). The tube was caped and microwaved for 4.5 hrs at 150° C., The reaction mixture was diluted with EA (100 ml). The organic layer was washed with brine and concentrated. The residue was purified by 100 g silica column, eluting with 5% MeOH in DCM (containing 0.1% TEA) to provide the title compound (923 mg, yield: 67%). MS: 328 [M+H]⁺. ¹H NMR (400 MHz, DMSO-d₆) δ 9.17 (dd, J=4.1, 1.8 Hz, 1H), 8.61 (dd, J=8.8, 1.8 Hz, 1H), 8.33 (d, J=1.5 Hz, 1H), 7.89 (dd, J=8.8, 4.1 Hz, 1H), 4.78 (t, J=5.6 Hz, 1H), 3.99 (t, J=8.1 Hz, 1H), 3.88 (dd, J=10.6, 5.6 Hz, 1H), 3.53 (ddd, J=13.7, 9.7, 3.6 Hz, 1H), 3.49-3.35 (m, 3H), 2.74 (dt, J=25.2, 11.1 Hz, 2H), 2.51 (t, J=2.0 Hz, 1H), 1.18 (dd, J=6.1, 1.5 Hz, 3H).

### Example 384: (1,1-Dioxo-hexahydro-1lambda6-thiopyran-4-yl)-[(2S,6R)-6-methyl-4-(8-trifluoromethyl-[1,7]naphthyridin-5-yl)-morpholin-2-ylmethyl]-amine

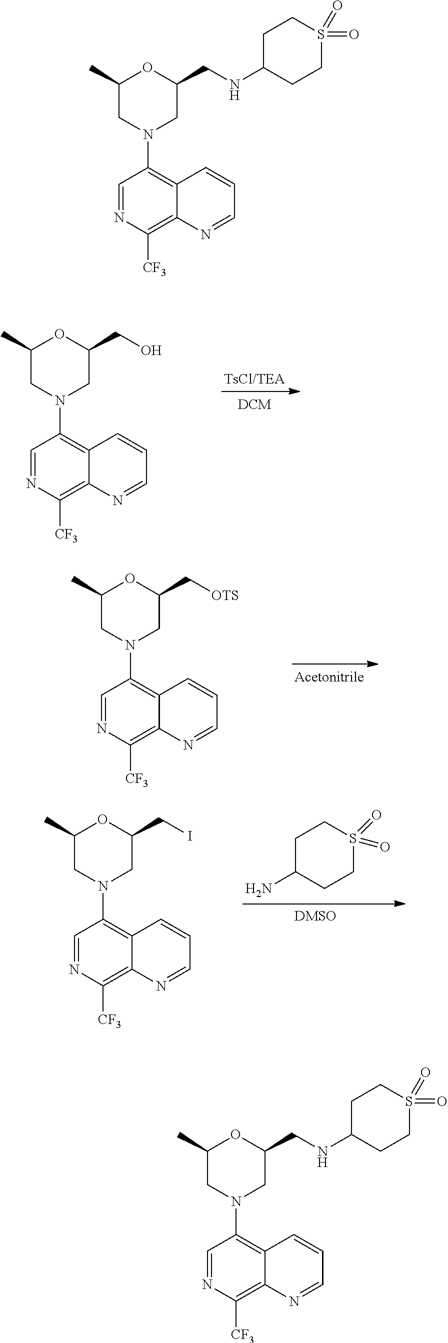

Toluene-4-sulfonic acid (2R,6R)-6-methyl-4-(8-trifluoromethyl-[1,7]naphthyridin-5-yl)-morpholin-2-ylmethyl ester: To a stirred solution of [(2R,6R)-6-Methyl-4-(8-trifluoromethyl-[1,7]naphthyridin-5-yl)-morpholin-2-yl]-methanol (923 mg; 2.82 mmol; 1.0 eq.) in DCM (2.70 ml) was added p-toluenesulfonyl chloride (645.16 mg; 3.38 mmol; 1.20 eq.) at room temperature, followed by TEA (0.79 ml; 5.64 mmol; 2.0 eq.). The mixture was stirred at room RT for 4 hr, until the reaction was completed. The reaction was diluted with EA (100 ml), The organic layer was washed with brine, dried over Na₂SO₄, and concentrated to yield the title compound as yellow solid (1200 mg, yield: 88%), which was directly used for the next step reaction. MS: 432 [M+H]⁺.

5-((2R,6R)-2-Iodomethyl-6-methyl-morpholin-4-yl)-8-trifluoromethyl-[1,7]naphthyridine: To a 25-mL vial, was placed Toluene-4-sulfonic acid (2R,6R)-6-methyl-4-(8-trifluoromethyl-[1,7]naphthyridin-5-yl)-morpholin-2-ylmethyl ester (1358 mg; 2.82 mmol; 1.0 eq.), sodium iodide (2113 mg; 14.10 mmol; 5.0 eq.) and acetonitrile (15 ml). The mixture was then stirred at 80° C. overnight, until the reaction was completed. The reaction mixture was diluted with EA (100 ml) and aqueous NaHSO₃ (10%) solution (15 mL). The organic layer was washed with NaHCO₃ aq (5%), then brine, dried with Na₂SO₄ and concentrated to give the title compound as a yellow solid, which was directly carried to the next step reaction without purification. MS: 438 [M+H]⁺.

(1,1-Dioxo-hexahydro-1lambda6-thiopyran-4-yl)-[(2S,6R)-6-methyl-4-(8-trifluoromethyl-[1,7]naphthyridin-5-yl)-morpholin-2-ylmethyl]-amine: A mixture of 5-((2R,6R)-2-Iodomethyl-6-methyl-morpholin-4-yl)-8-trifluoromethyl-[1,7]naphthyridine (43 mg; 0.10 mmol; 1.0 eq.), 1,1-Dioxo-hexahydro-1lambda6-thiopyran-4-ylamine (102 mg; 0.69 mmol; 7.0 eq.) and DMSO (1 mL) in a 10 mL microwave tube was stirred at 80° C. for 3 hrs, until the reaction was completed. The crude was purified by prep-HPLC (basic, 10-60% ACN in water), to yield the title compound (7 mg; 16%). MS: 459 [M+H]⁺. ¹H NMR (400 MHz, Methanol-d₄) δ 9.10 (d, J=4.2 Hz, 1H), 8.67 (d, J=8.7 Hz, 1H), 8.29 (s, 1H), 7.83 (dd, J=8.7, 4.1 Hz, 1H), 4.09 (s, 2H), 3.47 (t, J=13.6 Hz, 2H), 3.18 (s, 2H), 3.08 (d, J=10.9 Hz, 2H), 2.94-2.84 (m, 1H), 2.84-2.67 (m, 3H), 2.25 (s, 2H), 2.06 (dd, J=12.7, 8.1 Hz, 2H), 1.29 (d, J=6.2 Hz, 3H).

### Example 385 (Isomer 1): ((S)-4-Methyl-morpholin-2-ylmethyl)-[(2S,6R)-6-methyl-4-(8-trifluoromethyl-[1,7]naphthyridin-5-yl)-morpholin-2-ylmethyl]-amine & Example 386 (Isomer 2): ((R)-4-Methyl-morpholin-2-ylmethyl)-[(2S,6R)-6-methyl-4-(8-trifluoromethyl-[1,7]naphthyridin-5-yl)-morpholin-2-ylmethyl]-amine

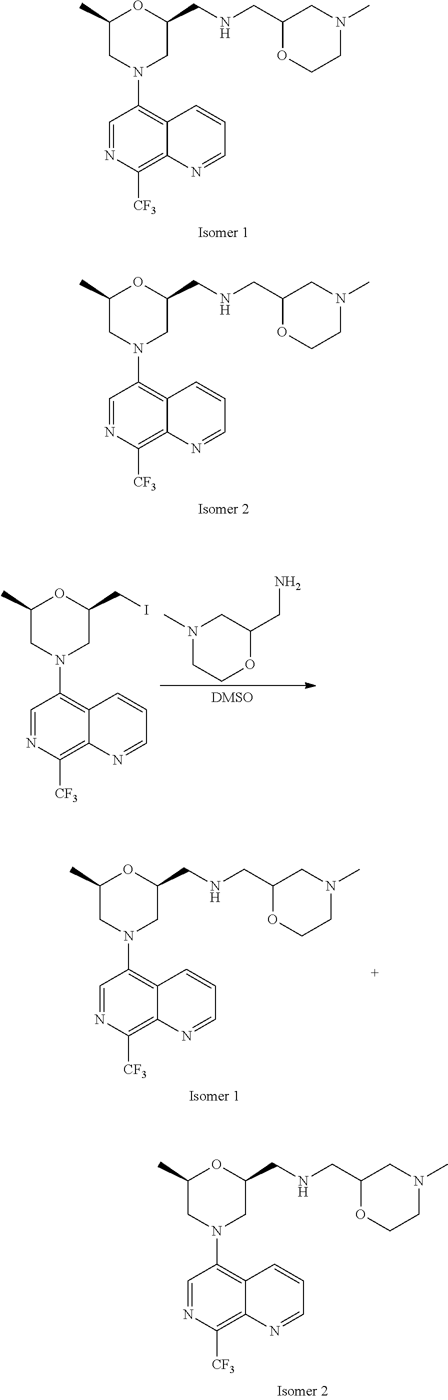

A mixture of 5-((2R,6R)-2-Iodomethyl-6-methyl-morpholin-4-yl)-8-trifluoromethyl-[1,7]naphthyridine (67 mg; 0.15 mmol; 1.0 eq.), (4-methylmorpholin-2-yl) methenamine (139 mg; 1.07 mmol; 7.0 eq.) and DMSO (1 ml) was stirred at 80° C. for 3 hrs. The crude was purified by prep HPLC ((mobile phase: 10-60% ACN/water (contained 0.1% ammonia)) to give the following two compounds.

Isomer 1: MS: 440 [M+H]⁺. ¹H NMR (400 MHz, DMSO-d₆) δ 9.16 (dd, J=4.1, 1.6 Hz, 1H), 8.62 (dd, J=8.6, 1.7 Hz, 1H), 8.32 (d, J=1.2 Hz, 1H), 7.95-7.80 (m, 1H), 3.95 (ddd, J=16.0, 11.4, 7.7 Hz, 2H), 3.75 (ddd, J=11.2, 3.3, 1.7 Hz, 1H), 3.55-3.36 (m, 4H), 2.85-2.54 (m, 7H), 2.14 (d, J=1.2 Hz, 3H), 1.93 (td, J=11.4, 3.3 Hz, 1H), 1.70 (t, J=10.6 Hz, 2H), 1.18 (d, J=6.2 Hz, 3H).

Isomer 2: MS: 440 [M+H]⁺. ¹H NMR (400 MHz, DMSO-d₆) δ 9.16 (dd, J=4.1, 1.6 Hz, 1H), 8.62 (dd, J=8.6, 1.7 Hz, 1H), 8.32 (d, J=1.2 Hz, 1H), 7.95-7.80 (m, 1H), 3.95 (ddd, J=16.0, 11.4, 7.7 Hz, 2H), 3.75 (ddd, J=11.2, 3.3, 1.7 Hz, 1H), 3.55-3.36 (m, 4H), 2.85-2.54 (m, 7H), 2.14 (d, J=1.2 Hz, 3H), 1.93 (td, J=11.4, 3.3 Hz, 1H), 1.70 (t, J=10.6 Hz, 2H), 1.18 (d, J=6.2 Hz, 3H).

### Example 387: 5-[(2R,6S)-2-Methyl-6-(4-methyl-piperazin-1-ylmethyl)-morpholin-4-yl]-8-trifluoromethyl-[1,7]naphthyridine

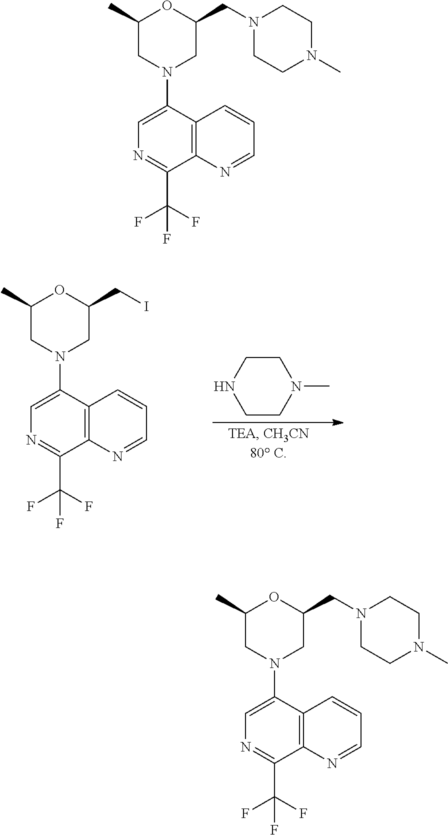

Into a 25 mL vial was placed 5-((2R,6R)-2-Iodomethyl-6-methyl-morpholin-4-yl)-8-trifluoromethyl-[1,7]naphthyridine (50.0 mg; 0.11 mmol; 1.0 eq.), 1-Methyl-piperazine (13.75 mg; 0.14 mmol; 1.20 eq.), MeCN (2.0 ml) and TEA (49.74 μl; 0.36 mmol; 3.13 eq.). The reaction solution was stirred for 10 h at 80° C. LCMS showed that the reaction was complete. 3 mL of DMSO were added and the resulting solution filtered with a Pall acrodisc 0.45 uM. The product was purified on reverse phase system using a gradient of 05-60% CH₃CN/H₂O (0.1% Ammonium Hydroxide) in 2 injections of 2 mL each. The desired fractions were evaporated to provide the title compound (28.0 mg; 60%) as a yellow solid. MS: 410 [M+H]⁺. ¹H NMR (400 MHz, Methanol-d4) δ 9.10 (s, 1H), 8.65 (d, J=8.7 Hz, 1H), 8.27 (s, 1H), 7.87-7.75 (m, 1H), 4.16 (p, J=5.3 Hz, 1H), 4.07 (dq, J=10.0, 6.4 Hz, 1H), 3.45 (t, J=13.6 Hz, 2H), 2.90-2.37 (m, 12H), 2.30 (s, 3H), 1.27 (d, J=6.2 Hz, 3H).

### Example 388: 5-((3R,5S)-3-Amino-5-trifluoromethyl-piperidin-1-yl)-2-oxo-1,2-dihydro-quinoline-8-carbonitrile

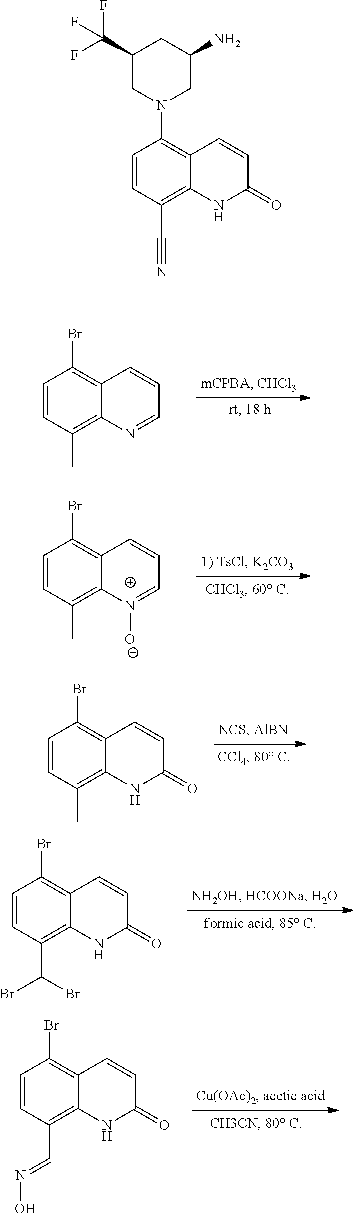

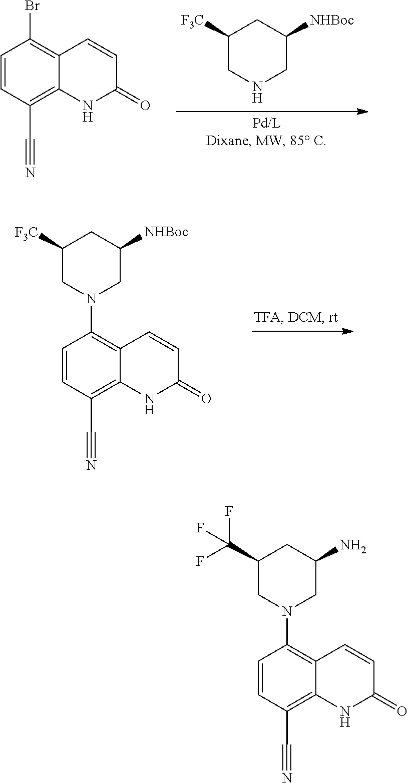 [SMILES: N#Cc1ccc(Br)c2ccc(=O)[nH]c12 ; CC(C)(C)OC(=O)N[C@H]1CNC[C@@H](C(F)(F)F)C1 ; CC(C)(C)OC(=O)N[C@@H]1C[C@H](C(F)(F)F)CN(c2ccc(C#N)c3[nH]c(=O)ccc23)C1 ; N#Cc1ccc(N2C[C@H](N)C[C@H](C(F)(F)F)C2)c2ccc(=O)[nH]c12]

5-Bromo-8-methyl-quinoline 1-oxide: In a 50 mL flask, to a solution of 5-bromo-8-methylquinoline (2000.0 mg; 9.01 mmol; 1.0 eq.) in anhydrous Trichloromethane (20.0 ml) was added 3-Chloro-benzenecarboperoxoic acid (2486.53 mg; 10.81 mmol; 1.20 eq.) at 0° C. portionwise. The mixture was stirred at room temperature overnight. DCM (50 mL) was added and then was washed with 5% aqueous NaHSO₃, saturated aqueous NaHCO₃, dried (Na₂SO₄), filtered and concentrated under reduced pressure to give the title compound (2200.0 mg; crude). MS: 238 [M+H]⁺.

5-Bromo-8-methyl-1H-quinolin-2-one: P-toluenesulfonyl chloride (1513.45 mg; 7.94 mmol; 1.50 eq.) and 10% aqueous potassium carbonate (40.0 ml) were added to a stirred solution of 5-Bromo-8-methyl-quinoline 1-oxide (1800.0 mg; 5.29 mmol; 1.0 eq.) in chloroform (30.0 ml). The mixture was stirred at room temperature for 3 hours. Water 50 mL was added and extracted with chloroform (3×20 ml). The combined organic phases were dried over Na₂SO₄ and then evaporated in vacuo. The residue was dissolved in DCM (20 mL), absorbed on a PuriFlash 50 g column and purified by chromatography (Hexanes-AcOEt, gradient 90-10% to 20-80% for 18 minutes. The pure fractions were concentrated under reduced pressure to give the title compound (360.0 mg; 29%). MS: 238, 240 [M+H]⁺.

5-Bromo-8-dibromomethyl-1H-quinolin-2-one: To a mixture of 5-Bromo-8-methyl-1H-quinolin-2-one (360.0 mg; 1.51 mmol; 1.0 eq.) and N-bromosuccinimide (570.88 mg; 3.18 mmol; 2.10 eq.) in CCl₄ (10.0 ml), was added 2,2′-azobis(2-methylpropionitrile) (37.24 mg; 0.23 mmol; 0.15 eq.). The resulting solution was stirred at 80° C. overnight. After cooling to rt, the precipitate was filtered off and the filtrate was evaporated to give the title compound (598.0 mg; crude) as a yellow solid. MS:395, 397 [M+H]⁺.

5-Bromo-2-oxo-1,2-dihydro-quinoline-8-carbaldehyde oxime: A mixture of 5-Bromo-8-dibromomethyl-1H-quinolin-2-one (598.0 mg; 1.36 mmol; 1.0 eq.), Sodium formate (253.04 mg; 3.53 mmol; 2.60 eq.), H₂O (1.10 ml; 61.18 mmol; 45.0 eq.), HCOOH (10.0 ml; 265.07 mmol; 194.97 eq.) and NH₂OH.HCl (119.34 mg; 1.63 mmol; 1.20 eq.) in a 100 ml seal tube was stirred at 85° C. for 2 hours. LCMS indicated a mixture of desired oxyme and aldehyde (2:1 ratio respectively). The reaction was concentrated and dissolved in hot ethyl acetate. The precipitated was filtered out. The mother liquid was concentrated and dry to give a mixture 2:1 of crude 5-Bromo-2-oxo-1,2-dihydro-quinoline-8-carbaldehyde oxime (320.0 mg; crude) and 5-Bromo-2-oxo-1,2-dihydro-quinoline-8-carbaldehyde. MS: 267 [M+H]⁺.

5-Bromo-2-oxo-1,2-dihydro-quinoline-8-carbonitrile: Hazard analysis: In a 200 mL pear flask fitted with a condenser, 5-Bromo-2-oxo-1,2-dihydro-quinoline-8-carbaldehyde oxime (480.0 mg; 1.44 mmol; 1.0 eq.) and copper(ii) acetate monohydrate (28.71 mg; 0.14 mmol; 0.10 eq.) were suspended in anhydrous MeCN (2.0 ml). Acetic acid (411.54 μl; 7.19 mmol; 5.0 eq.) was added to the beige suspension and the reaction mixture was heated to reflux for 3 hours. LCMS showed the reaction was complete. The reaction was filtered through celite. The filtrate was evaporated to give 5-Bromo-2-oxo-1,2-dihydro-quinoline-8-carbonitrile (436.0 mg; crude) as a yellow solid. MS:250 [M+H]⁺.

[(3R,5S)-1-(8-Cyano-2-oxo-1,2-dihydro-quinolin-5-yl)-5-trifluoromethyl-piperidin-3-yl]-carbamic acid tert-butyl ester: In a 10 mL microwave vial, was added 5-Bromo-2-oxo-1,2-dihydro-quinoline-8-carbonitrile (168.0 mg; 0.54 mmol; 1.0 eq.), ((3R,5S)-5-Trifluoromethyl-piperidin-3-yl)-carbamic acid tert-butyl ester (173.72 mg; 0.65 mmol; 1.20 eq.), methanesulfonato(2-dicyclohexylphosphino-2′,6′-di-i-propoxy-1,1′-biphenyl)(2′-amino-1,1′-biphenyl-2-yl)palladium(ii) (45.13 mg; 0.05 mmol; 0.10 eq.), 2-dicyclohexylphosphino-2′,6′-di-i-propoxy-1,1′-biphenyl (25.18 mg; 0.05 mmol; 0.10 eq.) and cesium carbonate (351.64 mg; 1.08 mmol; 2.0 eq.) and anhydrous tert-butanol (12.0 ml). The tube was sealed and flushed with nitrogen for 15 min and the cream suspension was microwaved at 100° C. for 5 hours. The reaction mixture was filtered through celite and concentrated under reduced pressure. The residue was suspended in DCM and absorbed on a PuriFlash celite 5 g column then purified by chromatography on a PuriFlash 25 g (Hexanes-AcOEt 10% for 5 column volumes, Hexanes-AcOEt 30-70% for 18 minutes. The pure fractions were concentrated under reduced pressure and the light-yellow oil was dried under vacuo to give the title compound (57.0 mg; 24%). MS: 437 [M+H]⁺.

5-((3R,5S)-3-Amino-5-trifluoromethyl-piperidin-1-yl)-2-oxo-1,2-dihydro-quinoline-8-carbonitrile: [(3R,5S)-1-(8-Cyano-2-oxo-1,2-dihydro-quinolin-5-yl)-5-trifluoromethyl-piperidin-3-yl]-carbamic acid tert-butyl ester (55.0 mg; 0.13 mmol; 1.0 eq.) was dissolved in dichloromethane (1.0 ml). TFA (0.50 ml) was added to reaction mixture. The resulting solution was stirred for two hours. The volatils were evaporated, the residue dissolved in methanol and filtered through a SiliaPrep™ SPE Cartridges Carbonate (1 g; 6 mL). The filtrate was evaporated to give the title compound (34.80 mg; 82%) as a yellow gum. MS: 337 [M+H]⁺. ¹H NMR (400 MHz, Methanol-d₄) δ 8.11 (d, J=8.2 Hz, 1H), 7.85 (d, J=8.4 Hz, 1H), 7.04 (d, J=8.4 Hz, 1H), 6.68 (d, J=9.8 Hz, 1H), 3.51 (t, J=11.0 Hz, 2H), 3.20 (d, J=10.0 Hz, 1H), 2.89 (d, J=9.8 Hz, 2H), 2.57 (d, J=11.1 Hz, 1H), 2.34 (d, J=14.5 Hz, 1H), 1.37 (q, J=12.3, 11.9 Hz, 1H).

### Example 389: HEK/293 TLR7 Cell Assay

Into 384 CulturePlates (Corning 3765) was placed 5000 c/w of TLR7/NFKb HEK cells in 30 uL DMEM without Phenol red (gibco #31053-028) and 10% heat inactivated fetal bovine serum, 10% penicillin-streptomycin, and 2 mM L-Glutamine. The cells were incubated for 24 h at 37° C., 10% carbon dioxide and 90% relative humidity. 3 uL of controls, standards, and compounds were dispensed into wells, incubated for 30 min then 3 uL of R848 agonist (10 uM final concentration) was added in 20 mM Hepes. After incubation for 5 hours, they were allowed to stand at room temperature for 15 min. To this was added 10 uL of Steady-Glo substrate reagent and shake assay plate for 5 min at 1500 rpm. The assay plate was allowed to sit for 30 min at room temperature and then the plate was read on EnVision.

### Example 390: HEK/293 TLR8 Cell Assay

Into 384 CulturePlates (Corning 3765) was placed 5000 c/w of TLR7/NFKb HEK cells in 30 uL DMEM without Phenol red (gibco #31053-028) and 10% heat inactivated fetal bovine serum, 1% penicillin-streptomycin, and 2 mM L-Glutamine. The cells were incubated for 24 h at 37° C., 10% carbon dioxide and 90% relative humidity. 3 uL of controls, standards, and compounds were dispensed into wells, incubated for 30 min then 3 uL of R848 agonist (30 uM final concentration) was added in 20 mM Hepes. After incubation for 5 hours, they were allowed to stand at room temperature for 15 min. To this was added 10 uL of Steady-Glo substrate reagent and shake assay plate for 5 min at 1500 rpm. The assay plate was allowed to sit for 30 min at room temperature and then the plate was read on EnVision.

Results are given in the following table.

| Example # | TLR7 potency | TLR8 potency |
|---|---|---|
| 1 | A | A |
| 2 | A | A |
| 3 | C | A |
| 4 | C | A |
| 5 | A | A |
| 6 | A | A |
| 7 | A | A |
| 8 | A | A |
| 9 | A | B |
| 10 | C | C |
| 11 | C | C |
| 12 | C | B |
| 13 | A | A |
| 14 | A | A |
| 15 | A | A |
| 16 | A | A |
| 17 | A | A |
| 18 | A | A |
| 19 | B | A |
| 20 | A | A |
| 21 | A | A |
| 22 | A | A |
| 23 | A | A |
| 24 | B | A |
| 25 | A | A |
| 26 | A | A |
| 27 | A | A |
| 28 | A | A |
| 29 | A | A |
| 30 | A | A |
| 31 | A | A |
| 32 | A | A |
| 33 | A | A |
| 34 | A | A |
| 35 | C | C |
| 36 | A | A |
| 37 | A | A |
| 38 | C | B |
| 39 | A | A |
| 40 | C | B |
| 41 | A | A |
| 42 | B | B |
| 43 | A | A |
| 44 | A | B |
| 45 | A | A |
| 46 | B | A |
| 47 | A | A |
| 48 | A | A |
| 49 | A | A |
| 50 | A | A |
| 51 | A | A |
| 52 | A | A |
| 53 | A | B |
| 54 | A | A |
| 55 | A | A |
| 56 | B | A |
| 57 | A | A |
| 58 | C | B |
| 59 | B | C |
| 60 | A | A |
| 61 | A | A |
| 62 | C | C |
| 63 | A | A |
| 64 | A | A |
| 65 | A | A |
| 66 | A | A |
| 67 | A | A |
| 68 | A | A |
| 69 | A | A |
| 70 | B | B |
| 71 | A | B |
| 72 | A | A |
| 73 | B | C |
| 74 | A | A |
| 75 | A | A |
| 76 | B | A |
| 77 | A | A |
| 78 | A | A |
| 79 | A | A |
| 80 | A | A |
| 81 | B | C |
| 82 | B | A |
| 83 | A | A |
| 84 | A | A |
| 85 | A | A |
| 86 | B | A |
| 87 | A | A |
| 88 | A | A |
| 89 | A | C |
| 90 | A | A |
| 91 | A | A |
| 92 | A | A |
| 93 | A | A |
| 94 | B | A |
| 95 | A | A |
| 96 | B | C |
| 97 | A | C |
| 98 | A | A |
| 99 | B | A |
| 100 | B | A |
| 101 | A | A |
| 102 | A | A |
| 103 | C | A |
| 104 | A | A |
| 105 | C | A |
| 106 | A | A |
| 107 | A | A |
| 108 | A | A |
| 109 | C | B |
| 110 | A | C |
| 111 | C | C |
| 112 | A | A |
| 113 | B | A |
| 114 | A | A |
| 115 | C | A |
| 116 | C | A |
| 117 | A | A |
| 118 | A | A |
| 119 | B | A |
| 120 | A | A |
| 121 | B | A |
| 122 | C | A |
| 123 | B | A |
| 124 | A | A |
| 125 | A | A |
| 126 | B | C |
| 127 | B | A |
| 128 | A | A |
| 129 | B | A |
| 130 | B | A |
| 131 | A | C |
| 132 | A | B |
| 133 | A | A |
| 134 | B | B |
| 135 | A | A |
| 136 | B | A |
| 137 | A | A |
| 138 | A | A |
| 139 | B | B |
| 140 | A | A |
| 141 | A | A |
| 142 | B | B |
| 143 | A | B |
| 144 | B | A |
| 145 | A | A |
| 146 | C | A |
| 147 | C | A |
| 148 | B | A |
| 149 | C | B |
| 150 | A | A |
| 151 | A | A |
| 152 | B | B |
| 153 | C | A |
| 154 | C | B |
| 155 | C | C |
| 156 | C | C |
| 157 | C | A |
| 158 | C | A |
| 159 | C | A |
| 160 | A | A |
| 161 | A | A |
| 162 | B | C |
| 163 | A | A |
| 164 | B | B |
| 165 | A | A |
| 166 | B | A |
| 167 | A | A |
| 168 | C | B |
| 169 | A | A |
| 170 | B | A |
| 171 | A | A |
| 172 | B | C |
| 173 | A | A |
| 174 | C | B |
| 175 | B | A |
| 176 | B | A |
| 177 | B | A |
| 178 | C | A |
| 179 | A | A |
| 180 | A | A |
| 181 | A | A |
| 182 | A | A |
| 183 | B | B |
| 184 | C | B |
| 185 | C | B |
| 186 | A | A |
| 187 | A | A |
| 188 | A | A |
| 189 | B | A |
| 190 | B | A |
| 191 | B | B |
| 192 | A | A |
| 193 | A | A |
| 194 | B | A |
| 195 | B | B |
| 196 | C | A |
| 197 | A | A |
| 198 | C | C |
| 199 | B | A |
| 200 | A | A |
| 201 | A | A |
| 202 | B | A |
| 203 | B | A |
| 204 | A | A |
| 205 | A | A |
| 206 | B | B |
| 207 | C | B |
| 208 | B | A |
| 209 | B | A |
| 210 | A | A |
| 211 | A | A |
| 212 | C | A |
| 213 | C | A |
| 214 | B | A |
| 215 | B | C |
| 216 | A | C |
| 217 | A | A |
| 218 | A | C |
| 219 | B | C |
| 220 | A | C |
| 221 | A | C |
| 222 | A | B |
| 223 | A | A |
| 224 | A | C |
| 225 | A | A |
| 226 | A | A |
| 227 | B | A |
| 228 | A | A |
| 229 | B | A |
| 230 | A | A |
| 231 | A | A |
| 232 | A | A |
| 233 | A | A |
| 234 | B | B |
| 235 | B | B |
| 236 | B | B |
| 237 | A | A |
| 238 | A | A |
| 239 | A | C |
| 240 | A | A |
| 241 | A | A |
| 242 | A | A |
| 243 | B | C |
| 244 | A | A |
| 245 | A | A |
| 246 | A | A |
| 247 | A | A |
| 248 | B | A |
| 249 | A | A |
| 250 | A | A |
| 251 | C | C |
| 252 | B | A |
| 253 | A | A |
| 254 | A | C |
| 255 | A | A |
| 256 | A | A |
| 257 | A | A |
| 258 | A | C |
| 259 | A | C |
| 260 | B | C |
| 261 | A | A |
| 262 | A | A |
| 263 | A | A |
| 264 | A | A |
| 265 | A | A |
| 266 | B | C |
| 267 | A | A |
| 268 | A | A |
| 269 | C | C |
| 270 | A | A |
| 271 | A | A |
| 272 | A | A |
| 273 | A | A |
| 274 | A | A |
| 275 | A | A |
| 276 | A | B |
| 277 | A | A |
| 278 | C | C |
| 279 | B | C |
| 280 | B | C |
| 281 | C | C |
| 282 | B | C |
| 283 | C | C |
| 284 | A | B |
| 285 | C | C |
| 286 | C | C |
| 287 | B | B |
| 288 | A | A |
| 289 | A | A |
| 290 | A | A |
| 291 | A | A |
| 292 | A | A |
| 293 | A | A |
| 294 | A | A |
| 295 | A | A |
| 296 | A | A |
| 297 | A | A |
| 298 | A | A |
| 299 | A | A |
| 300 | A | B |
| 301 | A | A |
| 302 | C | C |
| 303 | A | A |
| 304 | A | A |
| 305 | A | A |
| 306 | A | A |
| 307 | A | A |
| 308 | A | A |
| 309 | A | A |
| 310 | A | A |
| 311 | A | A |
| 312 | A | C |
| 313 | A | A |
| 314 | C | C |
| 315 | A | A |
| 316 | B | C |
| 317 | A | B |
| 318 | C | C |
| 319 | A | A |
| 320 | C | C |
| 321 | B | C |
| 322 | A | A |
| 323 | A | A |
| 324 | A | A |
| 325 | A | A |
| 326 | B | A |
| 327 | A | A |
| 328 | A | B |
| 329 | B | A |
| 330 | A | A |
| 331 | A | B |
| 332 | B | A |
| 333 | B | A |
| 334 | C | A |
| 335 | B | A |
| 336 | A | A |
| 337 | A | A |
| 338 | A | A |
| 339 | C | A |
| 340 | C | A |
| 341 | C | A |
| 342 | A | A |
| 343 | A | A |
| 344 | A | A |
| 345 | A | A |
| 346 | A | A |
| 347 | A | A |
| 348 | A | C |
| 349 | B | B |
| 350 | C | C |
| 351 | A | A |
| 352 | C | C |
| 352 | A | A |
| 354 | B | C |
| 355 | A | A |
| 356 | A | A |
| 357 | A | A |
| 358 | A | A |
| 359 | A | A |
| 360 | A | A |
| 361 | A | B |
| 362 | A | C |
| 363 | A | C |
| 364 | A | C |
| 365 | A | A |
| 366 | A | A |
| 367 | B | A |
| 368 | A | A |
| 369 | A | A |
| 370 | A | A |
| 371 | C | A |
| 372 | B | A |
| 373 | C | C |
| 374 | C | B |
| 375 | C | C |
| 376 | C | C |
| 377 | C | C |
| 378 | C | C |
| 379 | C | C |
| 380 | C | C |
| 381 | C | C |
| 382 | C | C |
| 383 | C | C |
| 384 | A | C |
| 385 | C | A |
| 386 | C | A |
| 387 | B | B |
| 388 | C | C |

A: IC50 < 75 nM
B: IC50: 75 nM-150 nM
C: IC50 > 150 nM

### Example 391. Pharmaceutical Preparations

(A) InJection vials: A solution of 100 g of an active ingredient according to the invention and 5 g of disodium hydrogen phosphate in 3 l of bidistilled water is adJusted to pH 6.5 using 2 N hydrochloric acid, sterile filtered, transferred into inJection vials, is lyophilized under sterile conditions and is sealed under sterile conditions. Each inJection vial contains 5 mg of active ingredient.

(B) Suppositories: A mixture of 20 g of an active ingredient according to the invention is melted with 100 g of soy lecithin and 1400 g of cocoa butter, is poured into moulds and is allowed to cool. Each suppository contains 20 mg of active ingredient.

(C) Solution: A solution is prepared from 1 g of an active ingredient according to the invention, 9.38 g of NaH₂PO₄.2H₂O, 28.48 g of Na₂HPO₄.12H₂O and 0.1 g of benzalkonium chloride in 940 ml of bidistilled water. The pH is adJusted to 6.8, and the solution is made up to 1 l and sterilized by irradiation. This solution could be used in the form of eye drops.

(D) Ointment: 500 mg of an active ingredient according to the invention is mixed with 99.5 g of Vaseline under aseptic conditions.

(E) Tablets: A mixture of 1 kg of an active ingredient according to the invention, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

(F) Coated tablets: Tablets are pressed analogously to Example E and subsequently are coated in a conventional manner with a coating of sucrose, potato starch, talc, tragacanth and dye.

(G) Capsules: 2 kg of an active ingredient according to the invention are introduced into hard gelatin capsules in a conventional manner in such a way that each capsule contains 20 mg of the active ingredient.

(H) Ampoules: A solution of 1 kg of an active ingredient according to the invention in 60 l of bidistilled water is sterile filtered, transferred into ampoules, is lyophilized under sterile conditions and is sealed under sterile conditions. Each ampoule contains 10 mg of active ingredient.

(I) Inhalation spray: 14 g of an active ingredient according to the invention are dissolved in 10 l of isotonic NaCl solution, and the solution is transferred into commercially available spray containers with a pump mechanism. The solution could be sprayed into the mouth or nose. One spray shot (about 0.1 ml) corresponds to a dose of about 0.14 mg.

While a number of embodiments of this invention are described herein, it is apparent that the basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I, 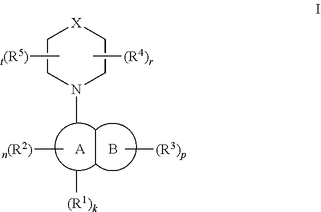
wherein:
Ring A is aryl or heteroaryl having 1-4 heteroatoms independently selected from nitrogen, oxygen, or sulfur; each of which is optionally substituted;
Ring B is aryl or heteroaryl having 1-4 heteroatoms independently selected from nitrogen, oxygen, or sulfur; each of which is optionally substituted;
R¹ is -Me, —CF₃, —OMe, —OEt, or —CN;
each R² is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R³ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
X is C(R⁴)₂, O, NR⁴, S, S(R⁴), or S(R⁴)₂;
each R⁴ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R⁵ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
k is 0 or 1;
n is 1, or 2;
p is 0, 1, or 2;
r is 0, 1, or 2; and
t is 0, 1, or 2;
or a derivative, solvate, hydrate, tautomer or stereoisomer thereof and/or a pharmaceutically acceptable salt of each of the foregoing, including mixtures thereof in all ratios.

2. The compound of claim 1, wherein Ring A is phenyl, pyridyl, pyrimidinyl, pyrazinyl, pyridazinyl, or triazinyl; each of which is optionally substituted, or a derivative, solvate, hydrate, tautomer, or stereoisomer thereof and/or a pharmaceutically acceptable salt of each of the foregoing, including mixtures thereof in all ratios.

3. The compound of claim 1, wherein Ring B is phenyl, pyridyl, pyrimidinyl, pyrazinyl, pyridazinyl, triazinyl, pyrrole, imidazole, isoxazole, oxazole, or thiazole; each of which is optionally substituted, or a derivative, solvate, hydrate, tautomer, or stereoisomer thereof and/or a pharmaceutically acceptable salt of each of the foregoing, including mixtures thereof in all ratios.

4. The compound of claim 1, wherein Ring A and Ring B is 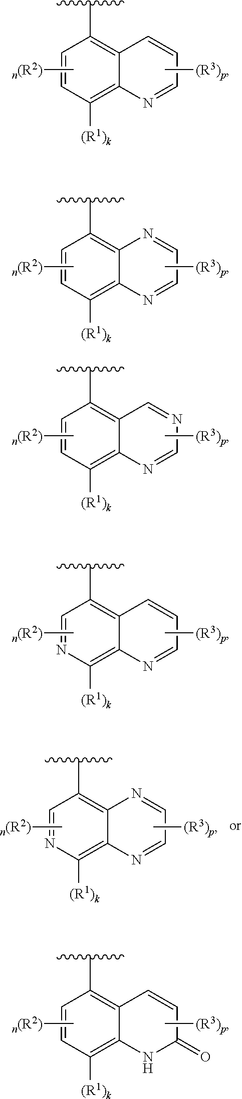
or a derivative, solvate, hydrate, tautomer, or stereoisomer thereof and/or a pharmaceutically acceptable salt of each of the foregoing, including mixtures thereof in all ratios.

5. The compound of claim 1, wherein X is C(R⁴)₂ or O, or a derivative, solvate, hydrate, tautomer, or stereoisomer thereof and/or a pharmaceutically acceptable salt of each of the foregoing, including mixtures thereof in all ratios.

6. The compound of claim 1, wherein each R⁴ is independently 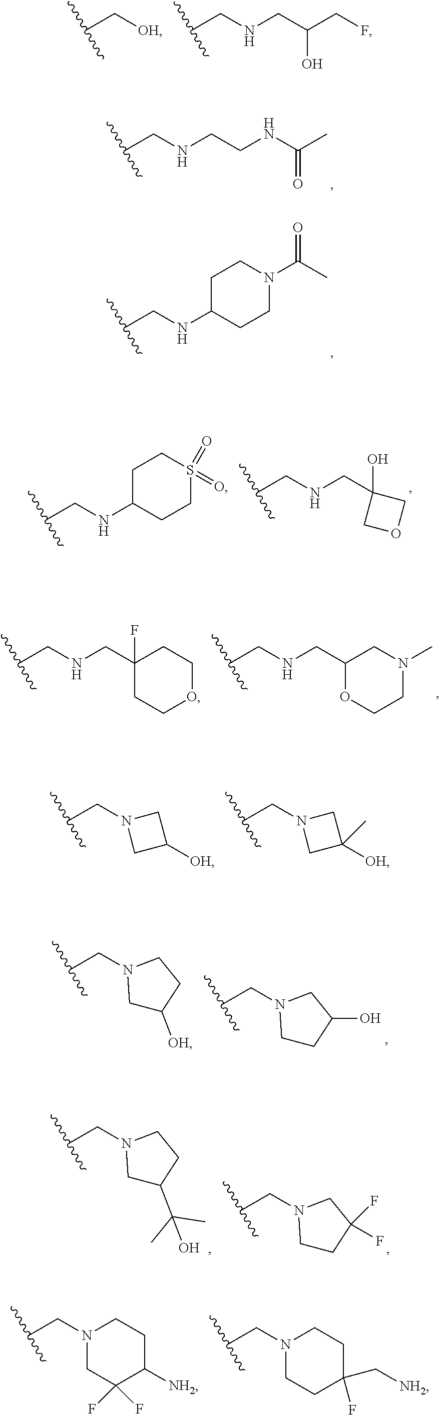 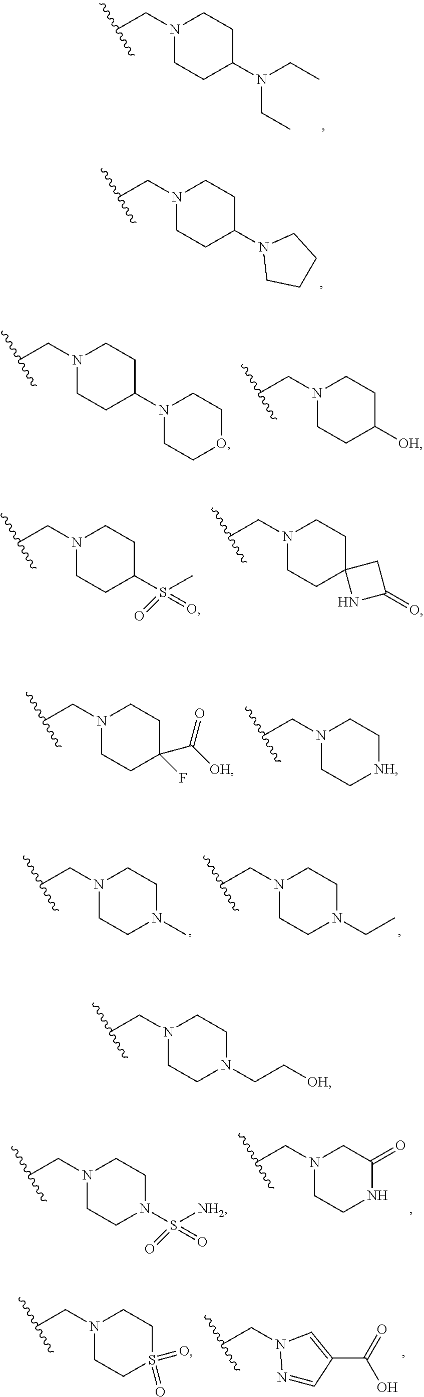 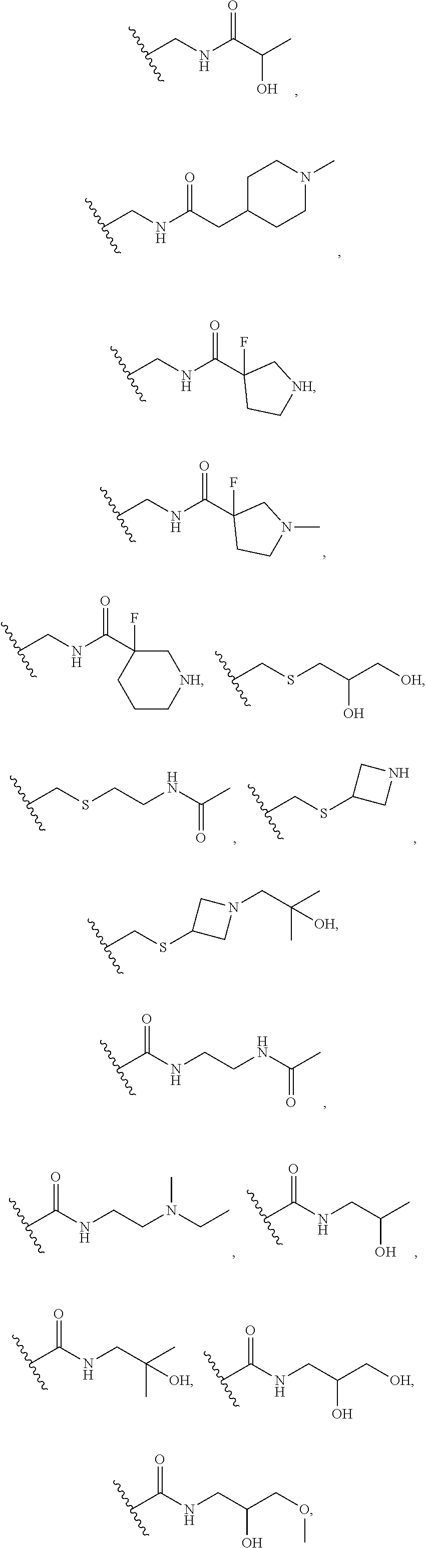 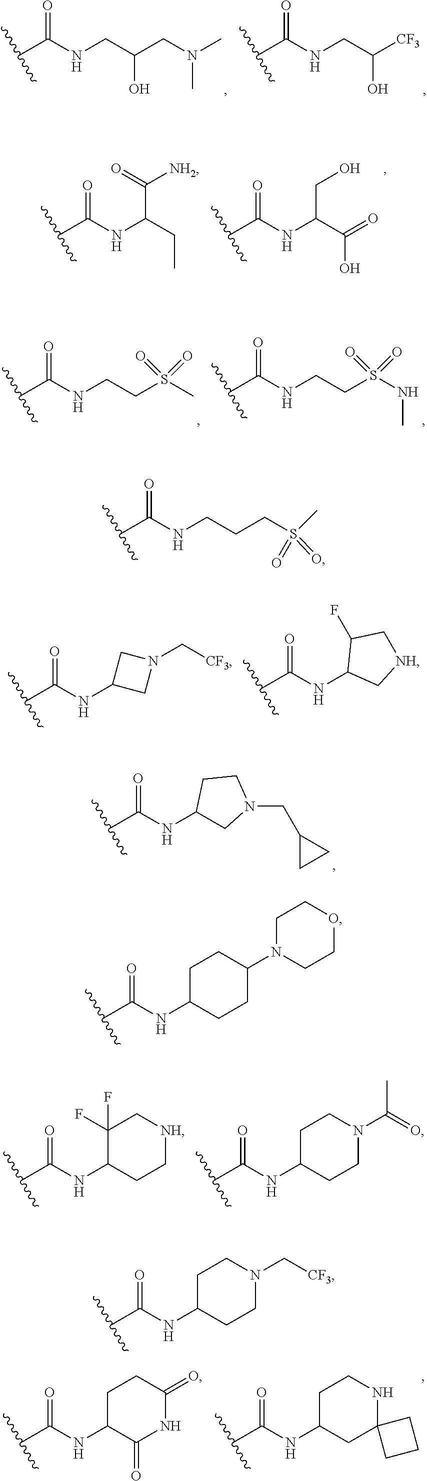 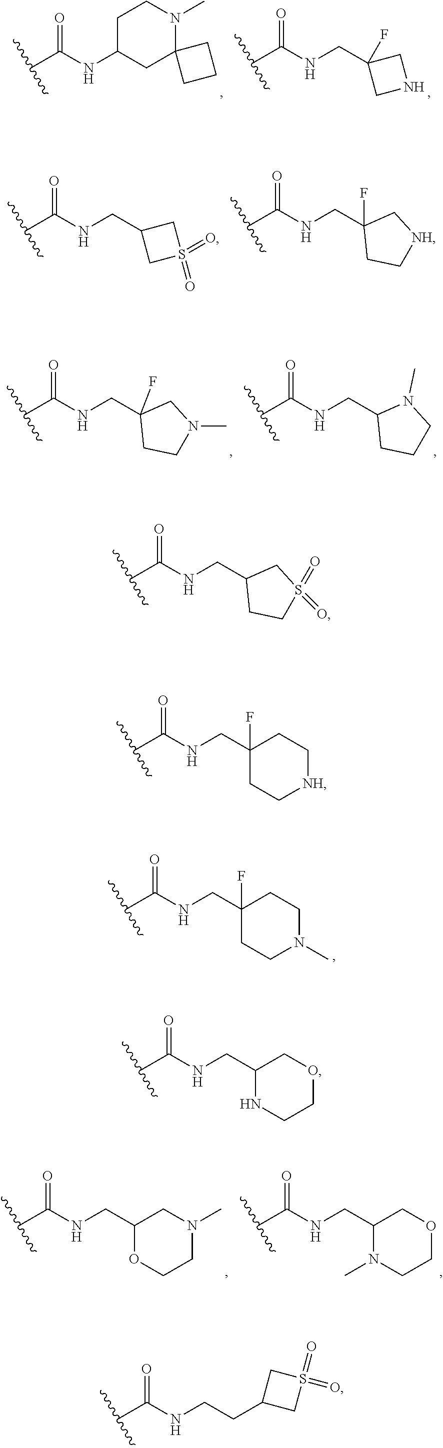 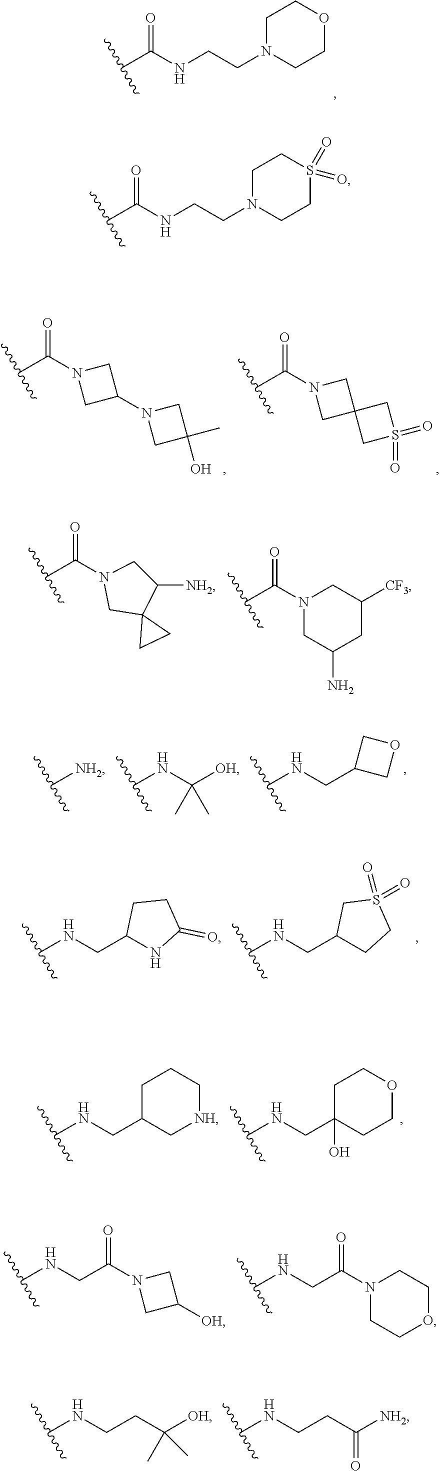 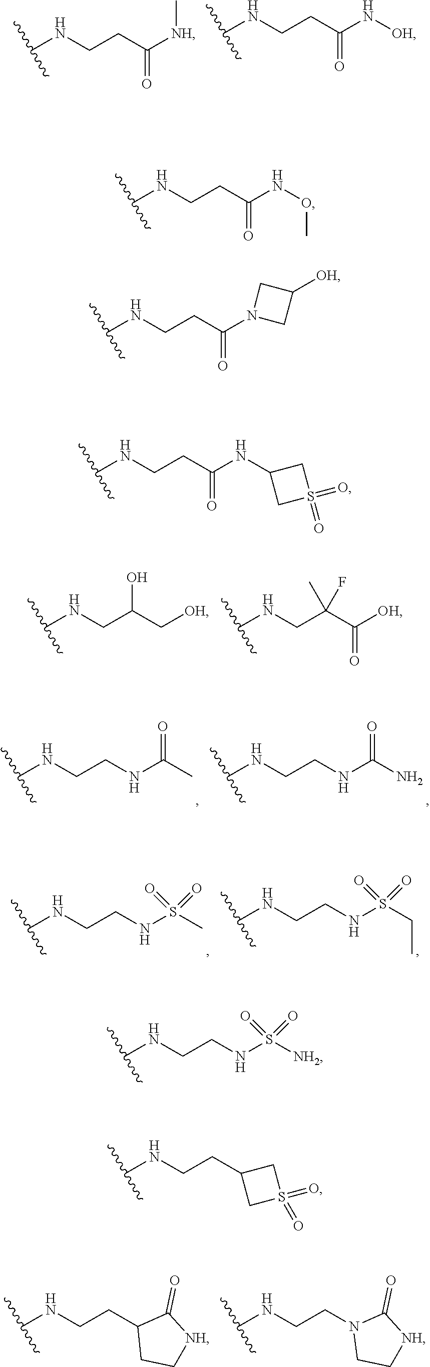 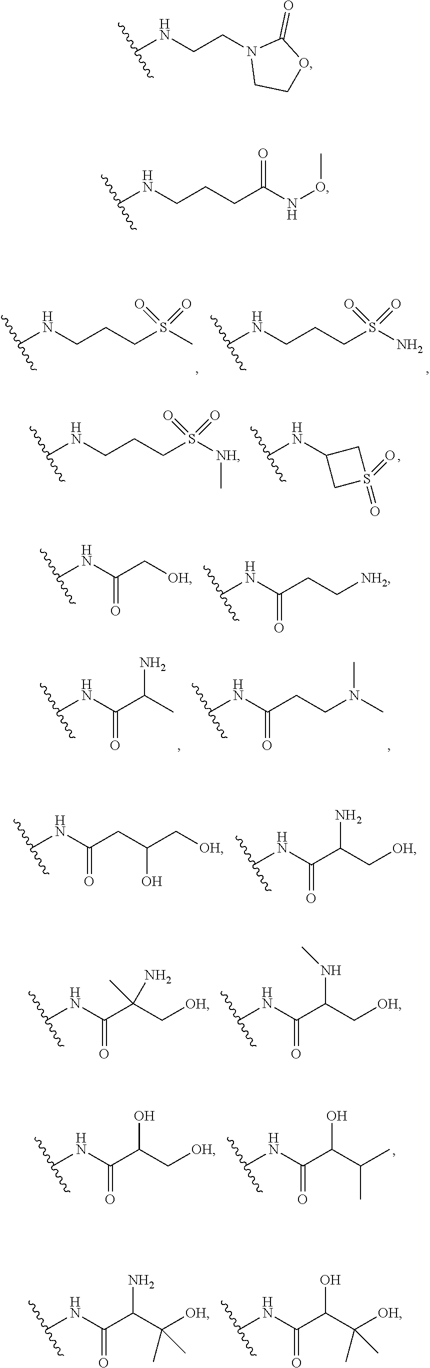 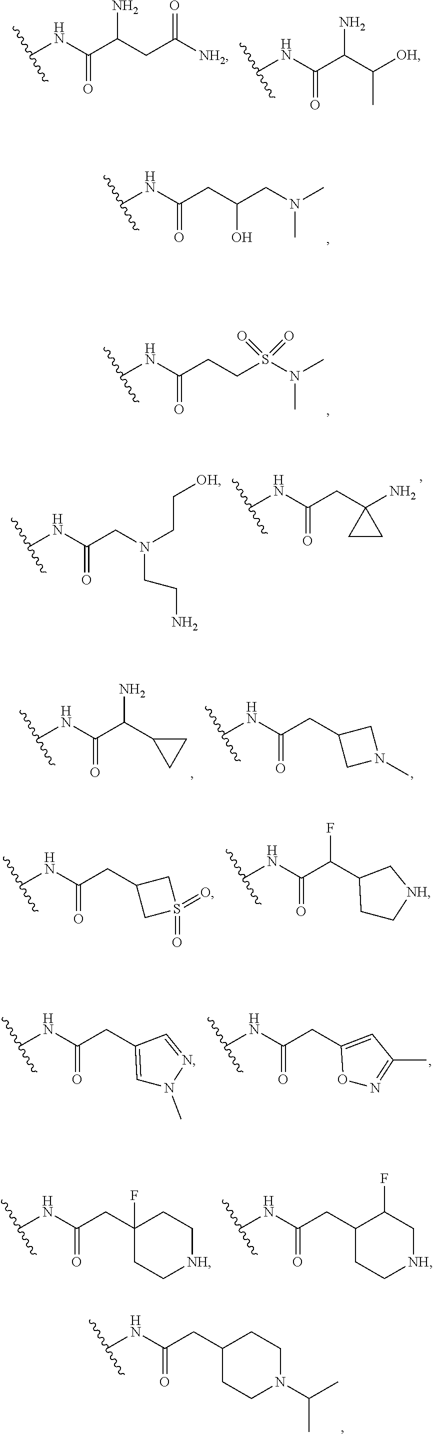 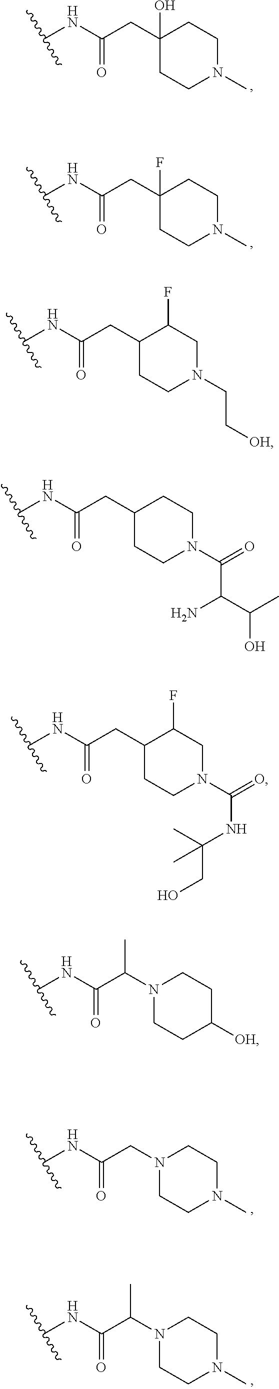 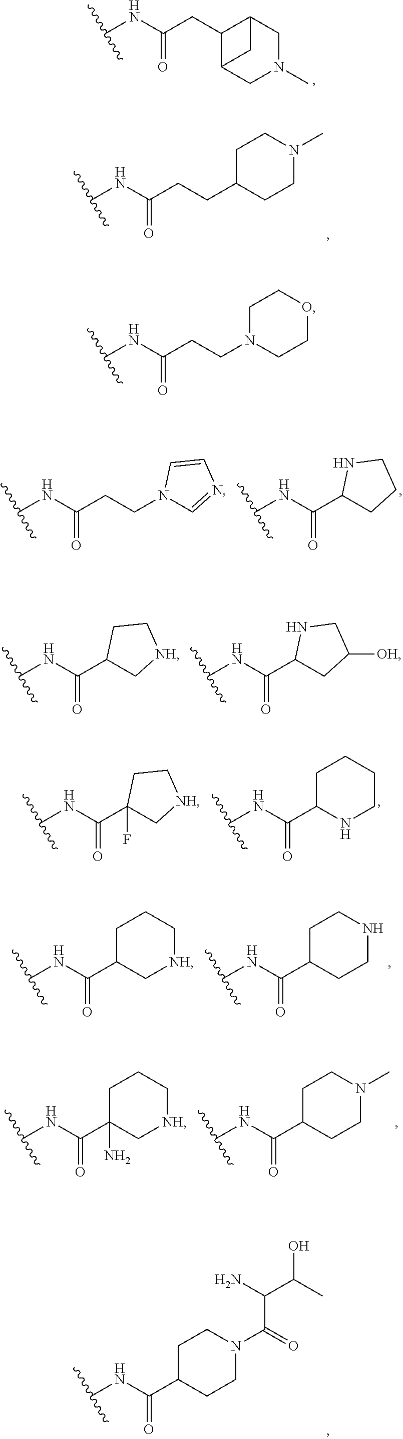 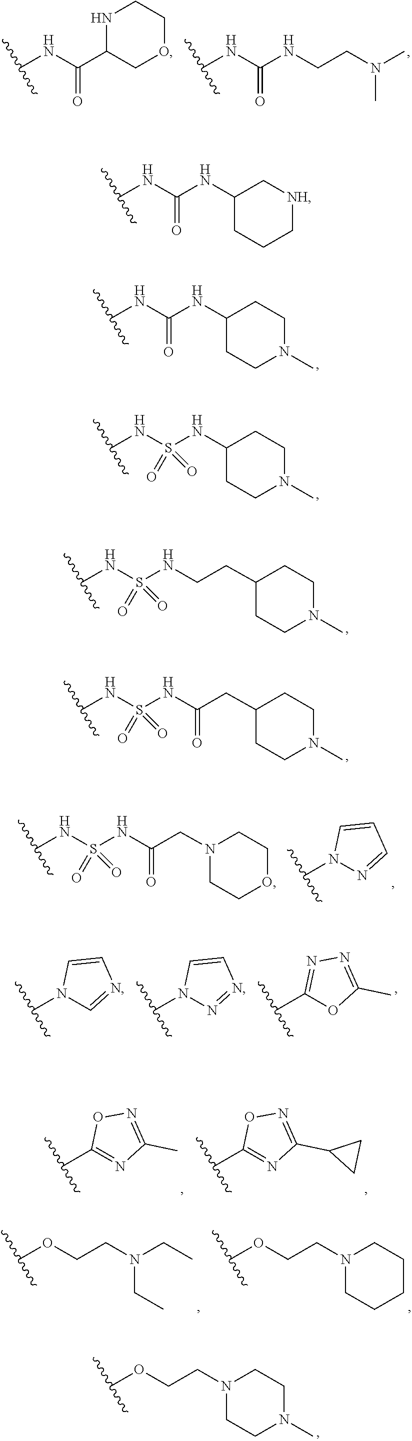
or a derivative, solvate, hydrate, tautomer, or stereoisomer thereof and/or a pharmaceutically acceptable salt of each of the foregoing, including mixtures thereof in all ratios.

7. The compound of claim 1, wherein each R⁵ is independently methyl, cyclopropyl, —F, or —CF₃, or a derivative, solvate, hydrate, tautomer, or stereoisomer thereof and/or a pharmaceutically acceptable salt of each of the foregoing, including mixtures thereof in all ratios.

8. The compound of claim 1, of formula I-a, 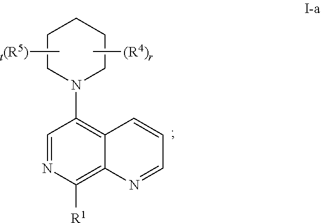
or a pharmaceutically acceptable salt thereof.

9. The compound of claim 8, wherein R¹ is —CF₃ or —OMe, or a pharmaceutically acceptable salt thereof.

10. The compound of claim 8, wherein each R⁴ is independently —H, C₁₋₆ aliphatic, —C(O)N(R)₂, —NRC(O)R, or —N(R)₂; each of which is optionally substituted, or a pharmaceutically acceptable salt thereof.

11. The compound of claim 8, wherein each R⁵ is independently methyl, —F, or —CF₃, or a pharmaceutically acceptable salt thereof.

12. The compound of claim 1, of formula I-b, 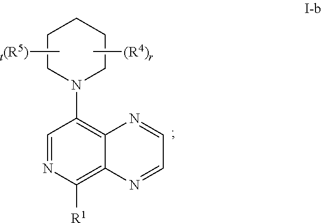
or a pharmaceutically acceptable salt thereof.

13. The compound of claim 12, wherein R¹ is —OMe or a pharmaceutically acceptable salt thereof.

14. The compound of claim 12, wherein each R⁴ is independently —H, C₁₋₆ aliphatic, —C(O)N(R)₂, —NRC(O)R, or —N(R)₂; each of which is optionally substituted, or a pharmaceutically acceptable salt thereof.

15. The compound of claim 12, wherein each R⁵ is independently methyl, —F, or —CF₃, or a pharmaceutically acceptable salt thereof.

16. The compound of claim 1, of formula I-c, 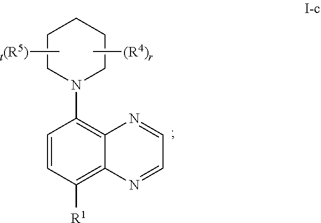
or a pharmaceutically acceptable salt thereof.

17. The compound of claim 16, wherein R¹ is —CN or a pharmaceutically acceptable salt thereof.

18. The compound of claim 16, wherein each R⁴ is independently —NRC(O)R, or —N(R)₂; each of which is optionally substituted, or a pharmaceutically acceptable salt thereof.

19. The compound of claim 16, wherein each R⁵ is independently methyl, —F, or —CF, or a pharmaceutically acceptable salt thereof.

20. The compound of claim 1, of formula I-d, 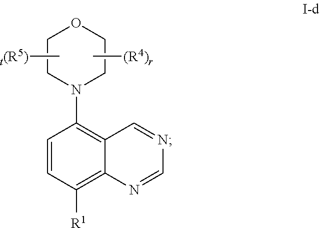
or a pharmaceutically acceptable salt thereof.

21. The compound of claim 20, wherein R¹ is —CN, or a pharmaceutically acceptable salt thereof.

22. The compound of claim 20, wherein each R⁴ is independently —H, C₁₋₆ aliphatic, —C(O)N(R)₂, —NRC(O)R, or —N(R)₂; each of which is optionally substituted, or a pharmaceutically acceptable salt thereof.

23. The compound of claim 20, wherein each R⁵ is independently methyl, —F, or —CF₃, or a pharmaceutically acceptable salt thereof.

24. The compound of claim 1, selected from Examples 1-388, or a pharmaceutically acceptable salt thereof.

25. A pharmaceutical composition comprising the compound of claim 1 or a pharmaceutically acceptable salt thereof, and a pharmaceutically acceptable adjuvant, carrier, or vehicle.

26. A method for inhibiting TLR7/8, or a mutant thereof, activity in a patient or in a biological sample, the method comprising:
administering to said patient or contacting said biological sample with the compound of claim 1 or a physiologically acceptable salt thereof.

27. A method for treating a TLR7/8-mediated disorder in a patient in need thereof, the method comprising:
administering to said patient the compound of claim 1 or a physiologically acceptable salt thereof.

28. The method of claim 27, wherein the disorder is selected from the group consisting of rheumatoid arthritis, psoriatic arthritis, osteoarthritis, systemic lupus erythematosus, lupus nephritis, ankylosing spondylitis, osteoporosis, systemic sclerosis, multiple sclerosis, psoriasis, type I diabetes, type II diabetes, inflammatory bowel disease, Crohn's disease, ulcerative colitis, hyperimmunoglobulinemia D, periodic fever syndrome, cryopyrin-associated periodic syndromes, Schnitzler's syndrome, systemic juvenile idiopathic arthritis, adult onset Still's disease, gout, pseudogout, SAPHO syndrome, Castleman's disease, sepsis, stroke atherosclerosis celiac disease, DIRA, Alzheimer's disease, Parkinson's disease, Sjogren's disease, polymyositis, dermatomyositis, and cancer.

29-30. (canceled)
